SEQUENCE LISTING (694 sequences)

SEQ ID NO: 1 - protein, 18 aa, synthetic construct
  REGION 1..18: note = Synthetic
KRTADGSEFESPKKKRKV

SEQ ID NO: 2 - protein, 17 aa, synthetic construct
  REGION 1..17: note = Synthetic
KRTADGSEFEPKKKRKV

SEQ ID NO: 3 - protein, 1367 aa, synthetic construct
  REGION 1..1367: note = Synthetic
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 4 - protein, 228 aa, synthetic construct
  REGION 1..228: note = Synthetic
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 5 - protein, 228 aa, synthetic construct
  REGION 1..228: note = Synthetic
SSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLK

SEQ ID NO: 6 - protein, 228 aa, synthetic construct
  REGION 1..228: note = Synthetic
SSETGPVAVDPTLRRRIEPHEFEVFFDPRELRKEACLLYEIKWGTSHKIWRNSGKNTTKHVEVNFIEKFTSERHFCPSISCSITWFLSWSPCWECSKAIREFLSQHPNVTLVIYVARLFQHMDQQNRQGLRDLVNSGVTIQIMTASEYDHCWRNFVNYPPGKEAHWPRYPPLWMKLYALELHAGILGLPPCLNILRRKQPQLTFFTIALQSCHYQRLPPHILWATGLK

SEQ ID NO: 7 - protein, 228 aa, synthetic construct
  REGION 1..228: note = Synthetic
SSETGPVAVDPTLRRRIEPEFFNRNYDPRELRKETYLLYEIKWGKESKIWRHTSNNRTQHAEVNFLENFFNELYFNPSTHCSITWFLSWSPCGECSKAIVEFLKEHPNVNLEIYVARLYLCEDERNRQGLRDLVNSGVTIRIMNLPDYNYCWRTFVSHQGGDEDYWPRHFAPWVRLYVLELYCIILGLPPCLNILRRKQPQLTFFTIALQSCHYQRLPPHILWATGLK

SEQ ID NO: 8 - protein, 228 aa, synthetic construct
  REGION 1..228: note = Synthetic
SSETGPVAVDPTLRRRIEPFYFQFNNDPRACRRKTYLCYELKQDGSTWVWKRTLHNKGRHAEICFLEKISSLEKLDPAQHYRITWYMSWSPCSNCAQKIVDFLKEHPHVNLRIYVARLYYHEEERYQEGLRNLRRSGVSIRVMDLPDFEHCWETFVDNGGGPFQPWPGLEELNSKQLSRRLQAGILGLPPCLNILRRKQPQLTFFTIALQSCHYQRLPPHILWATGLK

SEQ ID NO: 9 - protein, 228 aa, synthetic construct
  REGION 1..228: note = Synthetic
SSETGPVAVDPTLRRRIEPFHFQFNNDPRAYRRKTYLCYELKQDGSTWVLDRTLRNKGRHAEICFLDKINSWERLDPAQHYRVTWYMSWSPCSNCAQQVVDFLKEHPHVNLRIFAARLYYHEQRRYQEGLRSLRGSGVPVAVMTLPDFEHCWETFVDHGGRPFQPWDGLEELNSRSLSRRLQAGILGLPPCLNILRRKQPQLTFFTIALQSCHYQRLPPHILWATGLK

SEQ ID NO: 10 - protein, 84 aa, synthetic construct
  REGION 1..84: note = Synthetic
MTNLSDIIEKETGKQLVIQESILMLPEEVEEVIGNKPESDILVHTAYDESTDENVMLLTSDAPEYKPWALVIQDSNGENKIKML

SEQ ID NO: 11 - protein, 32 aa, synthetic construct
  REGION 1..32: note = Synthetic
SGGSSGGSSGSETPGTSESATPESSGGSSGGS

SEQ ID NO: 12 - protein, 1853 aa, synthetic construct
  REGION 1..1853: note = Synthetic
MKRTADGSEFESPKKKRKVSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 13 - protein, 1853 aa, synthetic construct
  REGION 1..1853: note = Synthetic
MKRTADGSEFESPKKKRKVSSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 14 - protein, 1853 aa, synthetic construct
  REGION 1..1853: note = Synthetic
MKRTADGSEFESPKKKRKVSSETGPVAVDPTLRRRIEPHEFEVFFDPRELRKEACLLYEIKWGTSHKIWRNSGKNTTKHVEVNFIEKFTSERHFCPSISCSITWFLSWSPCWECSKAIREFLSQHPNVTLVIYVARLFQHMDQQNRQGLRDLVNSGVTIQIMTASEYDHCWRNFVNYPPGKEAHWPRYPPLWMKLYALELHAGILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 15 - protein, 167 aa, synthetic construct
  REGION 1..167: note = Synthetic
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 16 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 17 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 18 - protein, 183 aa, unidentified
  source 1..183: note = Salmonella typhimurium
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 19 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 20 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 21 - protein, 161 aa, unidentified
  source 1..161: note = Caulobacter crescentus
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 22 - protein, 179 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 23 - protein, 166 aa, synthetic construct
  REGION 1..166: note = Synthetic
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 24 - protein, 1800 aa, synthetic construct
  REGION 1..1800: note = Synthetic
MKRTADGSEFESPKKKRKVMSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRTADGSEFEPKKKRKV

SEQ ID NO: 25 - protein, 10 aa, synthetic construct
  REGION 1..10: note = Synthetic
SGGSGGSGGS

SEQ ID NO: 26 - protein, 4 aa, synthetic construct
  REGION 1..4: note = Synthetic
SGGS

SEQ ID NO: 27 - DNA, 54 bp, synthetic construct
  misc_feature 1..54: note = Synthetic
  source 1..54: mol_type = other DNA
aaacggacagccgacggaagcgagttcgagtcaccaaagaagaagcggaaagtc

SEQ ID NO: 28 - DNA, 51 bp, synthetic construct
  misc_feature 1..51: note = Synthetic
  source 1..51: mol_type = other DNA
aaaagaaccgccgacggcagcgaattcgagcccaagaagaagaggaaagtc

SEQ ID NO: 29 - DNA, 4101 bp, synthetic construct
  misc_feature 1..4101: note = Synthetic
  source 1..4101: mol_type = other DNA
gacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 30 - DNA, 4101 bp, synthetic construct
  misc_feature 1..4101: note = Synthetic
  source 1..4101: mol_type = other DNA
gacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgac

SEQ ID NO: 31 - DNA, 96 bp, synthetic construct
  misc_feature 1..96: note = Synthetic
  source 1..96: mol_type = other DNA
tctggaggatctagcggaggatcctctggcagcgagacaccaggaacaagcgagtcagcaacaccagagagcagtggcggcagcagcggcggcagc

SEQ ID NO: 32 - DNA, 96 bp, synthetic construct
  misc_feature 1..96: note = Synthetic
  source 1..96: mol_type = other DNA
tctggaggatctagcggaggatcctctggaagcgagacaccaggcacaagcgagtccgccacaccagagagctccggcggctcctccggaggatcc

SEQ ID NO: 33 - DNA, 96 bp, synthetic construct
  misc_feature 1..96: note = Synthetic
  source 1..96: mol_type = other DNA
tccggaggatctagcggaggctcctctggctctgagacacctggcacaagcgagagcgcaacacctgaaagcagcgggggcagcagcggggggtca

SEQ ID NO: 34 - DNA, 30 bp, synthetic construct
  misc_feature 1..30: note = Synthetic
  source 1..30: mol_type = other DNA
agcggcgggagcggcgggagcggggggagc

SEQ ID NO: 35 - DNA, 30 bp, synthetic construct
  misc_feature 1..30: note = Synthetic
  source 1..30: mol_type = other DNA
agcggaggatccggaggatctggaggcagc

SEQ ID NO: 36 - DNA, 12 bp, synthetic construct
  misc_feature 1..12: note = Synthetic
  source 1..12: mol_type = other DNA
tctggcggctca

SEQ ID NO: 37 - DNA, 684 bp, Rattus rattus
  source 1..684: mol_type = genomic DNA
tcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaag

SEQ ID NO: 38 - DNA, 684 bp, synthetic construct
  misc_feature 1..684: note = Synthetic
  source 1..684: mol_type = other DNA
agcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcagtcttgtcactatcagcggctgcctcctcatattctgtgggctacaggccttaaa

SEQ ID NO: 39 - DNA, 684 bp, synthetic construct
  misc_feature 1..684: note = Synthetic
  source 1..684: mol_type = other DNA
tcatcagaaacaggaccagtcgccgtggacccaacactgaggagaaggattgagccccatgaatttgaagtctttttcgaccccagggagctgaggaaggaggcatgcctgctgtacgagatcaagtggggcacaagccacaagatctggcgcaacagcggcaagaacaccacaaagcacgtggaagtgaatttcatcgagaagtttacctccgagcggcacttctgcccctctatcagctgttccatcacatggtttctgtcttggagcccttgctgggagtgttccaaggccatccgcgagttcctgtctcagcaccctaacgtgaccctggtcatctacgtggcccggctgtttcaacacatggaccagcagaacaggcagggcctgcgcgatctggtgaattctggcgtgaccatccagatcatgacagcctcagagtacgaccattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcactatcgcactgcagagctgtcattaccagagactgcctcctcatatcctgtgggctacaggccttaaa

SEQ ID NO: 40 - DNA, 684 bp, synthetic construct
  misc_feature 1..684: note = Synthetic
  source 1..684: mol_type = other DNA
agcagcgagacaggacccgtggcagtggaccctacactgaggaggaggattgagcccgaatttttcaacaggaactacgaccccagagagctgcggaaggagacatacctgctgtatgagatcaagtggggcaaggagtccaagatctggcggcacacctctaacaatagaacacagcacgccgaggtgaacttcctggagaacttctttaatgagctgtactttaatccttctacccactgcagcatcacatggttcctgagctggtccccatgcggcgagtgttctaaggccatcgtggagtttctgaaggagcaccccaacgtgaatctggagatctacgtggccaggctgtatctgtgcgaggacgagaggaacaggcagggcctgcgggatctggtgaatagcggcgtgaccatcagaatcatgaacctgcctgactacaattattgttggcgcacattcgtgtcccaccagggaggcgacgaggattattggccaaggcactttgcaccatgggtgcgcctgtacgtgctggagctgtattgcatcatcctgggcctgcccccttgtctgaacatcctgcggagaaagcagccccagctgacattcttcaccatcgcactgcagagttgtcattaccagcgactgcctcctcatatcctgtgggctacaggccttaaa

SEQ ID NO: 41 - DNA, 684 bp, synthetic construct
  misc_feature 1..684: note = Synthetic
  source 1..684: mol_type = other DNA
tcatcagagaccggacctgtggcagtggacccaaccctgcgacggagaatcgagcccttttactttcagttcaacaacgacccaagagcctgccggagaaagacctacctgtgctatgagctgaagcaggacggctctacctgggtgtggaagcggacactgcacaacaagggcagacacgccgagatctgcttcctggagaagatcagctccctggagaagctggaccctgcccagcactacaggatcacatggtatatgtcttggagcccatgctccaactgtgcccagaagatcgtggattttctgaaggagcacccacacgtgaatctgcggatctacgtggccagactgtactatcacgaggaggagaggtatcaggagggcctgaggaacctgaggcgctccggcgtgtctatcagagtgatggacctgcccgatttcgagcactgctgggagacattcgtggataacggaggaggacctttccagccatggcccggcctggaggagctgaatagcaagcagctgtcccggagactgcaggcaggaatcctgggcctgcccccttgtctgaatatcctgaggcgcaagcagccccagctgacatttttcaccatcgcactgcagagttgtcattatcagcgactgcctcctcatatcctgtgggctacaggccttaaa

SEQ ID NO: 42 - DNA, 684 bp, synthetic construct
  misc_feature 1..684: note = Synthetic
  source 1..684: mol_type = other DNA
agcagcgagaccggacctgtggcagtggacccaaccctgagaagacgcattgagccatttcattttcagtttaacaacgaccccagagcctaccggagaaagacctacctgtgctatgagctgaagcaggacggctccacctgggtgctggatcggacactgagaaacaagggccggcacgccgagatctgtttcctggacaagatcaattcctgggagaggctggatcccgcccagcactaccgcgtgacatggtatatgagctggtccccttgctctaactgtgcccagcaggtggtggatttcctgaaggagcacccacacgtgaatctgcggatctttgccgccagactgtactatcacgagcagaggcgctatcaggagggcctgcggagcctgaggggaagcggagtgcctgtggccgtgatgaccctgccagacttcgagcactgctgggagacatttgtggatcacggcggccggccattccagccatgggacggcctggaggagctgaactctaggagcctgtcccggagactgcaggcaggaatcctgggcctgcccccttgtctgaatatcctgaggcgcaagcagccccagctgaccttttttaccatcgcactgcagagttgtcactaccagagactgcctcctcatatcctgtgggctacaggccttaaa

SEQ ID NO: 43 - DNA, 498 bp, synthetic construct
  misc_feature 1..498: note = Synthetic
  source 1..498: mol_type = other DNA
tctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgac

SEQ ID NO: 44 - DNA, 498 bp, synthetic construct
  misc_feature 1..498: note = Synthetic
  source 1..498: mol_type = other DNA
tctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgac

SEQ ID NO: 45 - DNA, 5559 bp, synthetic construct
  misc_feature 1..5559: note = Synthetic
  source 1..5559: mol_type = other DNA
atgaaacggacagccgacggaagcgagttcgagtcaccaaagaagaagcggaaagtctcctcagagactgggcctgtcgccgtcgatccaaccctgcgccgccggattgaacctcacgagtttgaagtgttctttgacccccgggagctgagaaaggagacatgcctgctgtacgagatcaactggggaggcaggcactccatctggaggcacacctctcagaacacaaataagcacgtggaggtgaacttcatcgagaagtttaccacagagcggtacttctgccccaataccagatgtagcatcacatggtttctgagctggtccccttgcggagagtgtagcagggccatcaccgagttcctgtccagatatccacacgtgacactgtttatctacatcgccaggctgtatcaccacgcagacccaaggaataggcagggcctgcgcgatctgatcagctccggcgtgaccatccagatcatgacagagcaggagtccggctactgctggcggaacttcgtgaattattctcctagcaacgaggcccactggcctaggtacccacacctgtgggtgcgcctgtacgtgctggagctgtattgcatcatcctgggcctgcccccttgtctgaatatcctgcggagaaagcagccccagctgaccttctttacaatcgccctgcagtcttgtcactatcagaggctgccaccccacatcctgtgggccacaggcctgaagtctggaggatctagcggaggatcctctggcagcgagacaccaggaacaagcgagtcagcaacaccagagagcagtggcggcagcagcggcggcagcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 46 - DNA, 5559 bp, synthetic construct
  misc_feature 1..5559: note = Synthetic
  source 1..5559: mol_type = other DNA
atgaaacggacagccgacggaagcgagttcgagtcaccaaagaagaagcggaaagtcagcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcagtcttgtcactatcagcggctgcctcctcatattctgtgggctacaggccttaaatctggaggatctagcggaggatcctctggcagcgagacaccaggaacaagcgagtcagcaacaccagagagcagtggcggcagcagcggcggcagcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 47 - DNA, 5559 bp, synthetic construct
  misc_feature 1..5559: note = Synthetic
  source 1..5559: mol_type = other DNA
atgaaacggacagccgacggaagcgagttcgagtcaccaaagaagaagcggaaagtctcatcagaaacaggaccagtcgccgtggacccaacactgaggagaaggattgagccccatgaatttgaagtctttttcgaccccagggagctgaggaaggaggcatgcctgctgtacgagatcaagtggggcacaagccacaagatctggcgcaacagcggcaagaacaccacaaagcacgtggaagtgaatttcatcgagaagtttacctccgagcggcacttctgcccctctatcagctgttccatcacatggtttctgtcttggagcccttgctgggagtgttccaaggccatccgcgagttcctgtctcagcaccctaacgtgaccctggtcatctacgtggcccggctgtttcaacacatggaccagcagaacaggcagggcctgcgcgatctggtgaattctggcgtgaccatccagatcatgacagcctcagagtacgaccattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcactatcgcactgcagagctgtcattaccagagactgcctcctcatatcctgtgggctacaggccttaaatctggaggatctagcggaggatcctctggcagcgagacaccaggaacaagcgagtcagcaacaccagagagcagtggcggcagcagcggcggcagcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 48 - DNA, 5409 bp, synthetic construct
  misc_feature 1..5409: note = Synthetic
  source 1..5409: mol_type = other DNA
atgaaacggacagccgacggaagcgagttcgagtcaccaaagaagaagcggaaagtctctgaagtcgagtttagccacgagtattggatgaggcacgcactgaccctggcaaagcgagcatgggatgaaagagaagtccccgtgggcgccgtgctggtgcacaacaatagagtgatcggagagggatggaacaggccaatcggccgccacgaccctaccgcacacgcagagatcatggcactgaggcagggaggcctggtcatgcagaattaccgcctgatcgatgccaccctgtatgtgacactggagccatgcgtgatgtgcgcaggagcaatgatccacagcaggatcggaagagtggtgttcggagcacgggacgccaagaccggcgcagcaggctccctgatggatgtgctgcaccaccccggcatgaaccaccgggtggagatcacagagggaatcctggcagacgagtgcgccgccctgctgagcgatttctttagaatgcggagacaggagatcaaggcccagaagaaggcacagagctccaccgactctggaggatctagcggaggatcctctggaagcgagacaccaggcacaagcgagtccgccacaccagagagctccggcggctcctccggaggatcctctgaggtggagttttcccacgagtactggatgagacatgccctgaccctggccaagagggcacgcgatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaacgcaaaaaccggcgccgcaggctccctgatggacgtgctgcactaccccggcatgaatcaccgcgtcgaaattaccgagggaatcctggcagatgaatgtgccgccctgctgtgctatttctttcggatgcctagacaggtgttcaatgctcagaagaaggcccagagctccaccgactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtc

SEQ ID NO: 49 - RNA, 21 nt, synthetic construct
  misc_feature 1..21: note = Synthetic
  source 1..21: mol_type = other RNA
gttagtccttaaaatgtaggg

SEQ ID NO: 50 - RNA, 21 nt, synthetic construct
  misc_feature 1..21: note = Synthetic
  source 1..21: mol_type = other RNA
gttcccggtcatgcactacag

SEQ ID NO: 51 - RNA, 20 nt, synthetic construct
  misc_feature 1..20: note = Synthetic
  source 1..20: mol_type = other RNA
cttgaccaatagccttgaca

SEQ ID NO: 52 - RNA, 20 nt, synthetic construct
  misc_feature 1..20: note = Synthetic
  source 1..20: mol_type = other RNA
atatttgcattgagatagtg

SEQ ID NO: 53 - protein, 1367 aa, synthetic construct
  REGION 1..1367: note = Synthetic
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 54 - protein, 1367 aa, synthetic construct; identical to SEQ ID NO: 3
  REGION 1..1367: note = Synthetic

SEQ ID NO: 55 - protein, 1367 aa, synthetic construct
  REGION 1..1367: note = Synthetic
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 56 - protein, 345 aa, unidentified
  source 1..345: note = Sulfolobus islandicus
MEVPLYNIFGDNYIIQVATEAENSTIYNNKVEIDDEELRNVLNLAYKIAKNNEDAAAERRGKAKKKKGEEGETTTSNIILPLSGNDKNPWTETLKCYNFPTTVALSEVFKNFSQVKECEEVSAPSFVKPEFYEFGRSPGMVERTRRVKLEVEPHYLIIAAAGWVLTRLGKAKVSEGDYVGVNVFTPTRGILYSLIQNVNGIVPGIKPETAFGLWIARKVVSSVTNPNVSVVRIYTISDAVGQNPTTINGGFSIDLTKLLEKRYLLSERLEAIARNALSISSNMRERYIVLANYIYEYLTGSKRLEDLLYFANRDLIMNLNSDDGKVRDLKLISAYVNGELIRGEG

SEQ ID NO: 57 - protein, 345 aa, unidentified
  source 1..345: note = Sulfolobus islandicus
MEVPLYNIFGDNYIIQVATEAENSTIYNNKVEIDDEELRNVLNLAYKIAKNNEDAAAERRGKAKKKKGEEGETTTSNIILPLSGNDKNPWTETLKCYNFPTTVALSEVFKNFSQVKECEEVSAPSFVKPEFYKFGRSPGMVERTRRVKLEVEPHYLIMAAAGWVLTRLGKAKVSEGDYVGVNVFTPTRGILYSLIQNVNGIVPGIKPETAFGLWIARKVVSSVTNPNVSVVSIYTISDAVGQNPTTINGGFSIDLTKLLEKRDLLSERLEAIARNALSISSNMRERYIVLANYIYEYLTGSKRLEDLLYFANRDLIMNLNSDDGKVRDLKLISAYVNGELIRGEG

SEQ ID NO: 58 - protein, 1193 aa, unidentified
  REGION 1..1193: note = uncultured Parcubacteria group bacterium
MSKRHPRISGVKGYRLHAQRLEYTGKSGAMRTIKYPLYSSPSGGRTVPREIVSAINDDYVGLYGLSNFDDLYNAEKRNEEKVYSVLDFWYDCVQYGAVFSYTAPGLLKNVAEVRGGSYELTKTLKGSHLYDELQIDKVIKFLNKKEISRANGSLDKLKKDIIDCFKAEYRERHKDQCNKLADDIKNAKKDAGASLGERQKKLFRDFFGISEQSENDKPSFTNPLNLTCCLLPFDTVNNNRNRGEVLFNKLKEYAQKLDKNEGSLEMWEYIGIGNSGTAFSNFLGEGFLGRLRENKITELKKAMMDITDAWRGQEQEEELEKRLRILAALTIKLREPKFDNHWGGYRSDINGKLSSWLQNYINQTVKIKEDLKGHKKDLKKAKEMINRFGESDTKEEAVVSSLLESIEKIVPDDSADDEKPDIPAIAIYRRFLSDGRLTLNRFVQREDVQEALIKERLEAEKKKKPKKRKKKSDAEDEKETIDFKELFPHLAKPLKLVPNFYGDSKRELYKKYKNAAIYTDALWKAVEKIYKSAFSSSLKNSFFDTDFDKDFFIKRLQKIFSVYRRFNTDKWKPIVKNSFAPYCDIVSLAENEVLYKPKQSRSRKSAAIDKNRVRLPSTENIAKAGIALARELSVAGFDWKDLLKKEEHEEYIDLIELHKTALALLLAVTETQLDISALDFVENGTVKDFMKTRDGNLVLEGRFLEMFSQSIVFSELRGLAGLMSRKEFITRSAIQTMNGKQAELLYIPHEFQSAKITTPKEMSRAFLDLAPAEFATSLEPESLSEKSLLKLKQMRYYPHYFGYELTRTGQGIDGGVAENALRLEKSPVKKREIKCKQYKTLGRGQNKIVLYVRSSYYQTQFLEWFLHRPKNVQTDVAVSGSFLIDEKKVKTRWNYDALTVALEPVSGSERVFVSQPFTIFPEKSAEEEGQRYLGIDIGEYGIAYTALEITGDSAKILDQNFISDPQLKTLREEVKGLKLDQRRGTFAMPSTKIARIRESLVHSLRNRIHHLALKHKAKIVYELEVSRFEEGKQKIKKVYATLKKADVYSEIDADKNLQTTVWGKLAVASEISASYTSQFCGACKKLWRAEMQVDETITTQELIGTVRVIKGGTLIDAIKDFMRPPIFDENDTPFPKYRDFCDKHHISKKMRGNSCLFICPFCRANADADIQASQTIALLRYVKEEKKVEDYFE

SEQ ID NO: 59 - protein, 1300 aa, unidentified
  source 1..1300: note = Francisella novicida
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 60 - protein, 1300 aa, synthetic construct
  REGION 1..1300: note = Synthetic
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 61 - protein, 1300 aa, synthetic construct
  REGION 1..1300: note = Synthetic
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 62 - protein, 1300 aa, synthetic construct
  REGION 1..1300: note = Synthetic
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 63 - protein, 1300 aa, synthetic construct
  REGION 1..1300: note = Synthetic
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 64 - protein, 1300 aa, synthetic construct
  REGION 1..1300: note = Synthetic
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 65 - protein, 1300 aa, synthetic construct
  REGION 1..1300: note = Synthetic
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 66 - protein, 1300 aa, synthetic construct
  REGION 1..1300: note = Synthetic
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIARGERHLAYYTLVDGKGNIIKQDTFNIIGNDRMKTNYHDKLAAIEKDRDSARKDWKKINNIKEMKEGYLSQVVHEIAKLVIEYNAIVVFADLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDAAANGAYHIGLKGLMLLGRIKNNQEGKKLNLVIKNEEYFEFVQNRNN

SEQ ID NO: 67 - protein, 887 aa, Natronobacterium gregoryi
MTVIDLDSTTTADELTSGHTYDISVTLTGVYDNTDEQHPRMSLAFEQDNGERRYITLWKNTTPKDVFTYDYATGSTYIFTNIDYEVKDGYENLTATYQTTVENATAQEVGTTDEDETFAGGEPLDHHLDDALNETPDDAETESDSGHVMTSFASRDQLPEWTLHTYTLTATDGAKTDTEYARRTLAYTVRQELYTDHDAAPVATDGLMLLTPEPLGETPLDLDCGVRVEADETRTLDYTTAKDRLLARELVEEGLKRSLWDDYLVRGIDEVLSKEPVLTCDEFDLHERYDLSVEVGHSGRAYLHINFRHRFVPKLTLADIDDDNIYPGLRVKTTYRPRRGHIVWGLRDECATDSLNTLGNQSVVAYHRNNQTPINTDLLDAIEAADRRVVETRRQGHGDDAVSFPQELLAVEPNTHQIKQFASDGFHQQARSKTRLSASRCSEKAQAFAERLDPVRLNGSTVEFSSEFFTGNNEQQLRLLYENGESVLTFRDGARGAHPDETFSKGIVNPPESFEVAVVLPEQQADTCKAQWDTMADLLNQAGAPPTRSETVQYDAFSSPESISLNVAGAIDPSEVDAAFVVLPPDQEGFADLASPTETYDELKKALANMGIYSQMAYFDRFRDAKIFYTRNVALGLLAAAGGVAFTTEHAMPGDADMFIGIDVSRSYPEDGASGQINIAATATAVYKDGTILGHSSTRPQLGEKLQSTDVRDIMKNAILGYQQVTGESPTHIVIHRDGFMNEDLDPATEFLNEQGVEYDIVEIRKQPQTRLLAVSDVQYDTPVKSIAAINQNEPRATVATFGAPEYLATRDGGGLPRPIQIERVAGETDIETLTRQVYLLSQSHIQVHNSTARLPITTAYADQASTHATKGYLVQTGAFESNVGFL

SEQ ID NO: 68 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 69 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 70 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 71 - DNA, 23 bp, synthetic construct
  misc_feature 1..23: note = Synthetic
  source 1..23: mol_type = other DNA
tttagtccttaaaatgtaggggg

SEQ ID NO: 72 - protein, 198 aa, Homo sapiens [sequence omitted]

SEQ ID NO: 73 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 74 - protein, 198 aa, unidentified
  source 1..198: note = Canis familiaris
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 75 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 76 - protein, 239 aa, Rattus rattus
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLTGWGALPAGLMSPARPSDYFYCWNTFVENHERTFKAWEGLHENSVRLSRRLRRILLPLYEVDDLRDAFRTLGL

SEQ ID NO: 77 - protein, 429 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 78 - protein, 429 aa, Rattus rattus
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 79 - protein, 370 aa, unidentified
  source 1..370: note = Rhesus macaque
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 80 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 81 - protein, 377 aa, Chlorocebus sabaeus
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 82 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 83 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 84 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 85 - protein, 396 aa, Rattus rattus
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFYWRKKFQKGLCTLWRSGIHVDVMDLPQFADCWTNFVNPQRPFRPWNELEKNSWRIQRRLRRIKESWGL

SEQ ID NO: 86 - protein, 227 aa, Bos taurus
DGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAEIRFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 87 - protein, 490 aa, Chlorocebus sabaeus
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 88 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 89 - protein, 190 aa, unidentified
  source 1..190: note = Troglodytes gorilla
MNPQIRNPMKAMYPGTFYFQFKNLWEANDRNETWLCFTVEGIKRRSVVSWKTGVFRNQVDSETHCHAERCFLSWFCDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 90 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 91 - protein, 202 aa, unidentified
  source 1..202: note = Rhesus macaque
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 92 - protein, 185 aa, Bos taurus
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 93 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 94 - protein, 210 aa, unidentified
  source 1..210: note = Rhesus macaque
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 95 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 96 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 97 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 98 - protein, 229 aa, Rattus rattus [sequence omitted]

SEQ ID NO: 99 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 100 - protein, 224 aa, Mus musculus
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 101 - protein, 224 aa, Rattus rattus
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 102 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 103 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 104 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQNQEN

SEQ ID NO: 105 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQ

SEQ ID NO: 106 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQ

SEQ ID NO: 107 - protein, 1368 aa, Streptococcus pyogenes [sequence omitted]

SEQ ID NO: 108 - protein, 83 aa, synthetic construct
  REGION 1..83: note = Synthetic
TNLSDIIEKETGKQLVIQESILMLPEEVEEVIGNKPESDILVHTAYDESTDENVMLLTSDAPEYKPWALVIQDSNGENKIKML

SEQ ID NO: 109 - protein, 120 aa, synthetic construct
  REGION 1..120: note = Synthetic
  VARIANT 5..120: note = may be absent
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 110 - protein, 150 aa, synthetic construct
  REGION 1..150: note = Synthetic
  VARIANT 6..150: note = may be absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 111 - protein, 150 aa, synthetic construct
  REGION 1..150: note = Synthetic
  VARIANT 6..150: note = may be absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 112 - protein, 90 aa, synthetic construct
  REGION 1..90: note = Synthetic
  VARIANT 4..90: note = may be absent
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 113 - protein, 120 aa, synthetic construct
  REGION 1..120: note = Synthetic
  VARIANT 5..120: note = may be absent
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 114 - protein, 16 aa, synthetic construct
  REGION 1..16: note = Synthetic
SGSETPGTSESATPES

SEQ ID NO: 115 - protein, 7 aa, synthetic construct
  REGION 1..7: note = Synthetic
PKKKRKV

SEQ ID NO: 116 - protein, 30 aa, synthetic construct
  REGION 1..30: note = Synthetic
MDSLLMNRRKFLYQFKNVRWAKGRRETYLC

SEQ ID NO: 117 - protein, 46 aa, synthetic construct
  REGION 1..46: note = Synthetic
  VARIANT 2: note = X can be any naturally occurring amino acid
  VARIANT 4..26: note = X can be any naturally occurring amino acid
  VARIANT 27..29: note = may be absent
  VARIANT 32..33: note = X can be any naturally occurring amino acid
  VARIANT 34..35: note = may be absent
HISXGLUXXXXXXXXXXXXXXXXXXXXXXXXXXPROCYSXXXXCYS

SEQ ID NO: 118 - protein, 30 aa, synthetic construct
  REGION 1..30: note = Synthetic
  VARIANT 2..30: note = may be absent
GGGGGGGGGGGGGGGGGGGGGGGGGGGGGG

SEQ ID NO: 119 - protein, 28 aa, synthetic construct
  REGION 1..28: note = Synthetic
  VARIANT 3..12: note = X can be any naturally occurring amino acid
LYSARGXXXXXXXXXXLYSLYSLYSLEU

SEQ ID NO: 120 - protein, 120 aa, synthetic construct
  REGION 1..120: note = Synthetic
  VARIANT 1: note = X can be any naturally occurring amino acid
  VARIANT 3..60: note = may be absent
XPROXPROXPROXPROXPROXPROXPROXPROXPROXPROXPROXPROXPROXPROXPROXPROXPROXPROXPROXPROXPROXPROXPROXPROXPROXPROXPROXPROXPROXPRO

SEQ ID NO: 121-132 - skipped (12 entries)

SEQ ID NO: 133 - DNA, 125 bp, synthetic construct
  misc_feature 1..125: note = Synthetic
  misc_difference 1..8: note = n is a, c, g, or t
  source 1..125: mol_type = other DNA
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 134 - DNA, 121 bp, synthetic construct
  misc_feature 1..121: note = Synthetic
  misc_difference 1..18: note = n is a, c, g, or t
  source 1..121: mol_type = other DNA
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 135 - DNA, 109 bp, synthetic construct
  misc_feature 1..109: note = Synthetic
  misc_difference 1..20: note = n is a, c, g, or t
  source 1..109: mol_type = other DNA
nnnnnnnnnnnnnnnnnnnngtttttgtactctcagaaatgcagaagctacaaagataaggcttcatgccgaaatcaacaccctgtcattttatggcagggtgtttttt

SEQ ID NO: 136 - DNA, 102 bp, synthetic construct
  misc_feature 1..102: note = Synthetic
  misc_difference 1..20: note = n is a, c, g, or t
  source 1..102: mol_type = other DNA
nnnnnnnnnnnnnnnnnnnngttttagagctagaaatagcaagttaaaataaggctagtccgttatcaacttgaaaaagtggcaccgagtcggtgctttttt

SEQ ID NO: 137 - DNA, 88 bp, synthetic construct
  misc_feature 1..88: note = Synthetic
  misc_difference 1..20: note = n is a, c, g, or t
  source 1..88: mol_type = other DNA
nnnnnnnnnnnnnnnnnnnngttttagagctagaaatagcaagttaaaataaggctagtccgttatcaacttgaaaaagtgttttttt

SEQ ID NO: 138 - DNA, 76 bp, synthetic construct
  misc_feature 1..76: note = Synthetic
  misc_difference 1..20: note = n is a, c, g, or t
  source 1..76: mol_type = other DNA
nnnnnnnnnnnnnnnnnnnngttttagagctagaaatagcaagttaaaataaggctagtccgttatcatttttttt

SEQ ID NO: 139 - RNA, 82 nt, synthetic construct
  misc_feature 1..82: note = Synthetic
  source 1..82: mol_type = other RNA
gttttagagctagaaatagcaagttaaaataaaggctagtccgttatcaacttgaaaaagtggcaccgagtcggtgcttttt

SEQ ID NO: 140 - DNA, 23 bp, synthetic construct
  misc_feature 1..23: note = Synthetic
  source 1..23: mol_type = other DNA
tatatgcatatttattacatcgg

SEQ ID NO: 141 - DNA, 23 bp, synthetic construct
  misc_feature 1..23: note = Synthetic
  source 1..23: mol_type = other DNA
ccgtcatgtgggtcctgaattgg

SEQ ID NO: 142 - DNA, 23 bp, synthetic construct
  misc_feature 1..23: note = Synthetic
  source 1..23: mol_type = other DNA
acactgaaagactccaggtcagg

SEQ ID NO: 143 - DNA, 23 bp, synthetic construct
  misc_feature 1..23: note = Synthetic
  source 1..23: mol_type = other DNA
gtcagaagagatgtggtcaatgg

SEQ ID NO: 144 - DNA, 23 bp, synthetic construct
  misc_feature 1..23: note = Synthetic
  source 1..23: mol_type = other DNA
tttaaagtgaagcagcatctggg

SEQ ID NO: 145 - DNA, 23 bp, synthetic construct
  misc_feature 1..23: note = Synthetic
  source 1..23: mol_type = other DNA
atttaaagtgaagcagcatctgg

SEQ ID NO: 146 - DNA, 23 bp, synthetic construct
  misc_feature 1..23: note = Synthetic
  source 1..23: mol_type = other DNA
actccatgacagtgtaattttgg

SEQ ID NO: 147 - DNA, 23 bp, synthetic construct
  misc_feature 1..23: note = Synthetic
  source 1..23: mol_type = other DNA
gcctggagaagccatccagcagg

SEQ ID NO: 148 - DNA, 23 bp, synthetic construct
  misc_feature 1..23: note = Synthetic
  source 1..23: mol_type = other DNA
ctcagacacactcattgatgagg

SEQ ID NO: 149 - DNA, 24 bp, synthetic construct
  misc_feature 1..24: note = Synthetic
  source 1..24: mol_type = other DNA
gaggcactgcccccaccatgagcg

SEQ ID NO: 150 - DNA, 23 bp, synthetic construct
  misc_feature 1..23: note = Synthetic
  source 1..23: mol_type = other DNA
gaacacaaagcatagactgcggg

SEQ ID NO: 151 - DNA, 23 bp, synthetic construct
  misc_feature 1..23: note = Synthetic
  source 1..23: mol_type = other DNA
ggcccagactgagcacgtgatgg

SEQ ID NO: 152 - DNA, 23 bp, synthetic construct
  misc_feature 1..23: note = Synthetic
  source 1..23: mol_type = other DNA
ggcactgcggctggaggtccggg

SEQ ID NO: 153 - DNA, 23 bp, synthetic construct
  misc_feature 1..23: note = Synthetic
  source 1..23: mol_type = other DNA
gtcatcttagtcattacctgagg

SEQ ID NO: 154 - DNA, 23 bp, synthetic construct
  misc_feature 1..23: note = Synthetic
  source 1..23: mol_type = other DNA
gagtccgagcagaagaagaaggg

SEQ ID NO: 155 - DNA, 24 bp, synthetic construct
  misc_feature 1..24: note = Synthetic
  source 1..24: mol_type = other DNA
gttagtccttaaaatgtagggggg

SEQ ID NO: 156 - DNA, 24 bp, synthetic construct
  misc_feature 1..24: note = Synthetic
  source 1..24: mol_type = other DNA
gttcccggtcatgcactacagagg

SEQ ID NO: 157 - DNA, 23 bp, synthetic construct
  misc_feature 1..23: note = Synthetic
  source 1..23: mol_type = other DNA
gagtatgaggcatagactgcagg

SEQ ID NO: 158 - DNA, 23 bp, synthetic construct
  misc_feature 1..23: note = Synthetic
  source 1..23: mol_type = other DNA
gatgagataatgatgagtcaggg

SEQ ID NO: 159 - DNA, 23 bp, synthetic construct
  misc_feature 1..23: note = Synthetic
  source 1..23: mol_type = other DNA
gaagatagagaatagactgctgg

SEQ ID NO: 160 - DNA, 23 bp, synthetic construct
  misc_feature 1..23: note = Synthetic
  source 1..23: mol_type = other DNA
gggaataaatcatagaatcctgg

SEQ ID NO: 161 - DNA, 23 bp, synthetic construct
  misc_feature 1..23: note = Synthetic
  source 1..23: mol_type = other DNA
cttgaccaatagccttgacaagg

SEQ ID NO: 162 - DNA, 23 bp, synthetic construct
  misc_feature 1..23: note = Synthetic
  source 1..23: mol_type = other DNA
atatttgcattgagatagtgtgg

SEQ ID NO: 163 - DNA, 25 bp, synthetic construct
  misc_feature 1..25: note = Synthetic
  source 1..25: mol_type = other DNA
caccgttcccggtcatgcactacag

SEQ ID NO: 164 - DNA, 25 bp, synthetic construct
  misc_feature 1..25: note = Synthetic
  source 1..25: mol_type = other DNA
aaacctgtagtgcatgaccgggaac

SEQ ID NO: 165 - DNA, 22 bp, synthetic construct
  misc_feature 1..22: note = Synthetic
  source 1..22: mol_type = other DNA
ggtgtttcgtcctttccacaag

SEQ ID NO: 166 - DNA, 55 bp, synthetic construct
  misc_feature 1..55: note = Synthetic
  source 1..55: mol_type = other DNA
gaacacaaagcatagactgcgttttagagctagaaatagcaagttaaaataaggc

SEQ ID NO: 167 - DNA, 55 bp, synthetic construct
  misc_feature 1..55: note = Synthetic
  source 1..55: mol_type = other DNA
ggcccagactgagcacgtgagttttagagctagaaatagcaagttaaaataaggc

SEQ ID NO: 168 - DNA, 55 bp, synthetic construct
  misc_feature 1..55: note = Synthetic
  source 1..55: mol_type = other DNA
ggcactgcggctggaggtgggttttagagctagaaatagcaagttaaaataaggc

SEQ ID NO: 169 - DNA, 55 bp, synthetic construct
  misc_feature 1..55: note = Synthetic
  source 1..55: mol_type = other DNA
gtcatcttagtcattacctggttttagagctagaaatagcaagttaaaataaggc

SEQ ID NO: 170 - DNA, 55 bp, synthetic construct
  misc_feature 1..55: note = Synthetic
  source 1..55: mol_type = other DNA
gagtccgagcagaagaagaagttttagagctagaaatagcaagttaaaataaggc

SEQ ID NO: 171 - DNA, 55 bp, synthetic construct
  misc_feature 1..55: note = Synthetic
  source 1..55: mol_type = other DNA
gagtatgaggcatagactgcgttttagagctagaaatagcaagttaaaataaggc

SEQ ID NO: 172 - DNA, 55 bp, synthetic construct
  misc_feature 1..55: note = Synthetic
  source 1..55: mol_type = other DNA
gatgagataatgatgagtcagttttagagctagaaatagcaagttaaaataaggc

SEQ ID NO: 173 - DNA, 55 bp, synthetic construct
  misc_feature 1..55: note = Synthetic
  source 1..55: mol_type = other DNA
gaagatagagaatagactgcgttttagagctagaaatagcaagttaaaataaggc

SEQ ID NO: 174 - DNA, 55 bp, synthetic construct
  misc_feature 1..55: note = Synthetic
  source 1..55: mol_type = other DNA
gggaataaatcatagaatccgttttagagctagaaatagcaagttaaaataaggc

SEQ ID NO: 175 - DNA, 56 bp, synthetic construct
  misc_feature 1..56: note = Synthetic
  source 1..56: mol_type = other DNA
gttagtccttaaaatgtaggggttttagagctagaaatagcaagttaaaataaggc

SEQ ID NO: 176 - DNA, 56 bp, synthetic construct
  misc_feature 1..56: note = Synthetic
  source 1..56: mol_type = other DNA
gcttgaccaatagccttgacagttttagagctagaaatagcaagttaaaataaggc

SEQ ID NO: 177 - DNA, 56 bp, synthetic construct
  misc_feature 1..56: note = Synthetic
  source 1..56: mol_type = other DNA
gatatttgcattgagatagtggttttagagctagaaatagcaagttaaaataaggc

SEQ ID NO: 178 - DNA, 58 bp, synthetic construct
  misc_feature 1..58: note = Synthetic
  misc_difference 34..37: note = n is a, c, g, or t
  source 1..58: mol_type = other DNA
acactctttccctacacgacgctcttccgatctnnnngcgaacacactgagacagaac

SEQ ID NO: 179 - DNA, 50 bp, synthetic construct
  misc_feature 1..50: note = Synthetic
  source 1..50: mol_type = other DNA
tggagttcagacgtgtgctcttccgatctgcactcctagttaggcttgtg

SEQ ID NO: 180 - DNA, 60 bp, synthetic construct
  misc_feature 1..60: note = Synthetic
  misc_difference 34..37: note = n is a, c, g, or t
  source 1..60: mol_type = other DNA
acactctttccctacacgacgctcttccgatctnnnngtccctggatggatgggctatgg

SEQ ID NO: 181 - DNA, 52 bp, synthetic construct
  misc_feature 1..52: note = Synthetic
  source 1..52: mol_type = other DNA
tggagttcagacgtgtgctcttccgatctggctttcccagacctgagttccc

SEQ ID NO: 182 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnncctggcctcactggatactc

SEQ ID NO: 183 - DNA, 52 bp, synthetic construct
  misc_feature 1..52: note = Synthetic
  source 1..52: mol_type = other DNA
tggagttcagacgtgtgctcttccgatctctgacaaaagaagtcctggtatc

SEQ ID NO: 184 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnnccagccccatctgtcaaact

SEQ ID NO: 185 - DNA, 54 bp, synthetic construct
  misc_feature 1..54: note = Synthetic
  source 1..54: mol_type = other DNA
tggagttcagacgtgtgctcttccgatcttgaatggattccttggaaacaatga

SEQ ID NO: 186 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnnatgtgggctgcctagaaagg

SEQ ID NO: 187 - DNA, 49 bp, synthetic construct
  misc_feature 1..49: note = Synthetic
  source 1..49: mol_type = other DNA
tggagttcagacgtgtgctcttccgatctcccagccaaacttgtcaacc

SEQ ID NO: 188 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnngaacccaggtagccagagac

SEQ ID NO: 189 - DNA, 49 bp, synthetic construct
  misc_feature 1..49: note = Synthetic
  source 1..49: mol_type = other DNA
tggagttcagacgtgtgctcttccgatcttcctttcaacccgaacggag

SEQ ID NO: 190 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnnacgtctcatatgccccttgg

SEQ ID NO: 191 - DNA, 51 bp, synthetic construct
  misc_feature 1..51: note = Synthetic
  source 1..51: mol_type = other DNA
tggagttcagacgtgtgctcttccgatctacgtaggaattttggtgggaca

SEQ ID NO: 192 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnncagctcagcctgagtgttga

SEQ ID NO: 193 - DNA, 48 bp, synthetic construct
  misc_feature 1..48: note = Synthetic
  source 1..48: mol_type = other DNA
tggagttcagacgtgtgctcttccgatctctcgtgggtttgtggttgc

SEQ ID NO: 194 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnnagagactgattgcgtggagt

SEQ ID NO: 195 - DNA, 49 bp, synthetic construct
  misc_feature 1..49: note = Synthetic
  source 1..49: mol_type = other DNA
tggagttcagacgtgtgctcttccgatctcactccagcctaggcaacaa

SEQ ID NO: 196 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnngtctgaggtcacacagtggg

SEQ ID NO: 197 - DNA, 49 bp, synthetic construct
  misc_feature 1..49: note = Synthetic
  source 1..49: mol_type = other DNA
tggagttcagacgtgtgctcttccgatctctgagagcagggaccacatc

SEQ ID NO: 198 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnntcacttcagcccaggagtat

SEQ ID NO: 199 - DNA, 49 bp, synthetic construct
  misc_feature 1..49: note = Synthetic
  source 1..49: mol_type = other DNA
tggagttcagacgtgtgctcttccgatcttctctttctctcccccaccc

SEQ ID NO: 200 - DNA, 57 bp, synthetic construct
  misc_feature 1..57: note = Synthetic
  misc_difference 34..37: note = n is a, c, g, or t
  source 1..57: mol_type = other DNA
acactctttccctacacgacgctcttccgatctnnnngggaggtggagagaggatgt

SEQ ID NO: 201 - DNA, 49 bp, synthetic construct
  misc_feature 1..49: note = Synthetic
  source 1..49: mol_type = other DNA
tggagttcagacgtgtgctcttccgatcttcctgaggtctaggaacccg

SEQ ID NO: 202 - protein, 1625 aa, synthetic construct
  REGION 1..1625: note = Synthetic
MKRTADGSEFESPKKKRKVSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSKRTADGSEFEPKKKRKV

SEQ ID NO: 203 - protein, 228 aa, Rattus rattus; identical to SEQ ID NO: 4

SEQ ID NO: 204 - protein, 228 aa, synthetic construct; identical to SEQ ID NO: 5
  REGION 1..228: note = Synthetic

SEQ ID NO: 205 - protein, 228 aa, synthetic construct; identical to SEQ ID NO: 6
  REGION 1..228: note = Synthetic

SEQ ID NO: 206 - protein, 228 aa, synthetic construct; identical to SEQ ID NO: 7
  REGION 1..228: note = Synthetic

SEQ ID NO: 207 - protein, 228 aa, synthetic construct; identical to SEQ ID NO: 8
  REGION 1..228: note = Synthetic

SEQ ID NO: 208 - protein, 228 aa, synthetic construct; identical to SEQ ID NO: 9
  REGION 1..228: note = Synthetic

SEQ ID NO: 209 - protein, 272 aa, synthetic construct
  REGION 1..272: note = Synthetic
GSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKSGGSPKKKRKV

SEQ ID NO: 210 - protein, 1800 aa, synthetic construct; identical to SEQ ID NO: 24
  REGION 1..1800: note = Synthetic

SEQ ID NO: 211 - DNA, 4875 bp, synthetic construct
  misc_feature 1..4875: note = Synthetic
  source 1..4875: mol_type = other DNA
atgaaacggacagccgacggaagcgagttcgagtcaccaaagaagaagcggaaagtctctggaggatctagcggaggatcctctggcagcgagacaccaggaacaagcgagtcagcaacaccagagagcagtggcggcagcagcggcggcagcgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgacagcggcgggagcggcgggagcggggggagcactaatctgagcgacatcattgagaaggagactgggaaacagctggtcattcaggagtccatcctgatgctgcctgaggaggtggaggaagtgatcggcaacaagccagagtctgacatcctggtgcacaccgcctacgacgagtccacagatgagaatgtgatgctgctgacctctgacgcccccgagtataagccttgggccctggtcatccaggattctaacggcgagaataagatcaagatgctgagcggaggatccggaggatctggaggcagcaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggcggctcaaaaagaaccgccgacggcagcgaattcgagcccaagaagaagaggaaagtc

SEQ ID NO: 212 - DNA, 684 bp, Rattus rattus; identical to SEQ ID NO: 37
  source 1..684: mol_type = genomic DNA

SEQ ID NO: 213 - DNA, 686 bp, synthetic construct
  misc_feature 1..686: note = Synthetic
  source 1..686: mol_type = other DNA
agcagtgaaaccggaccagtggcagtggacccaaccctgaggagacggattgagccccatgaatttgaagtgttctttgacccaagggagctgaggaaggagacatgcctgctgtacgagatcaagtggggcacaagccacaagatctggcgccacagctccaagaacaccacaaagcacgtggaagtgaatttcatcgagaagtttacctccgagcggcacttctgcccctctaccagctgttccatcacatggtttctgtcttggagcccttgcggcgagtgttccaaggccatcaccgagttcctgtctcagcaccctaacgtgaccctggtcatctacgtggcccggctgtatcaccacatggaccagcagaacaggcagggcctgcgcgatctggtgaattctggcgtgaccatccagatcatgacagccccagagtacgactattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcaccattgctctgcagtcttgtcactatcagcggctgcctcctcatattctgtgggctacaggcctttgaaa

SEQ ID NO: 214 - DNA, 686 bp, synthetic construct
  misc_feature 1..686: note = Synthetic
  source 1..686: mol_type = other DNA
tcatcagaaacaggaccagtcgccgtggacccaacactgaggagaaggattgagccccatgaatttgaagtctttttcgaccccagggagctgaggaaggaggcatgcctgctgtacgagatcaagtggggcacaagccacaagatctggcgcaacagcggcaagaacaccacaaagcacgtggaagtgaatttcatcgagaagtttacctccgagcggcacttctgcccctctatcagctgttccatcacatggtttctgtcttggagcccttgctgggagtgttccaaggccatccgcgagttcctgtctcagcaccctaacgtgaccctggtcatctacgtggcccggctgtttcaacacatggaccagcagaacaggcagggcctgcgcgatctggtgaattctggcgtgaccatccagatcatgacagcctcagagtacgaccattgctggcggaacttcgtgaattatccacctggcaaggaggcacactggccaagatacccacccctgtggatgaagctgtatgcactggagctgcacgcaggaatcctgggcctgcctccatgtctgaatatcctgcggagaaagcagccccagctgacatttttcactatcgcactgcagagctgtcattaccagagactgcctcctcatatcctgtgggctacaggcctttgaaa

SEQ ID NO: 215 - DNA, 686 bp, synthetic construct
  misc_feature 1..686: note = Synthetic
  source 1..686: mol_type = other DNA
agcagcgagacaggacccgtggcagtggaccctacactgaggaggaggattgagcccgaatttttcaacaggaactacgaccccagagagctgcggaaggagacatacctgctgtatgagatcaagtggggcaaggagtccaagatctggcggcacacctctaacaatagaacacagcacgccgaggtgaacttcctggagaacttctttaatgagctgtactttaatccttctacccactgcagcatcacatggttcctgagctggtccccatgcggcgagtgttctaaggccatcgtggagtttctgaaggagcaccccaacgtgaatctggagatctacgtggccaggctgtatctgtgcgaggacgagaggaacaggcagggcctgcgggatctggtgaatagcggcgtgaccatcagaatcatgaacctgcctgactacaattattgttggcgcacattcgtgtcccaccagggaggcgacgaggattattggccaaggcactttgcaccatgggtgcgcctgtacgtgctggagctgtattgcatcatcctgggcctgcccccttgtctgaacatcctgcggagaaagcagccccagctgacattcttcaccatcgcactgcagagttgtcattaccagcgactgcctcctcatatcctgtgggctacaggcctttgaaa

SEQ ID NO: 216 - DNA, 686 bp, synthetic construct
  misc_feature 1..686: note = Synthetic
  source 1..686: mol_type = other DNA
tcatcagagaccggacctgtggcagtggacccaaccctgcgacggagaatcgagcccttttactttcagttcaacaacgacccaagagcctgccggagaaagacctacctgtgctatgagctgaagcaggacggctctacctgggtgtggaagcggacactgcacaacaagggcagacacgccgagatctgcttcctggagaagatcagctccctggagaagctggaccctgcccagcactacaggatcacatggtatatgtcttggagcccatgctccaactgtgcccagaagatcgtggattttctgaaggagcacccacacgtgaatctgcggatctacgtggccagactgtactatcacgaggaggagaggtatcaggagggcctgaggaacctgaggcgctccggcgtgtctatcagagtgatggacctgcccgatttcgagcactgctgggagacattcgtggataacggaggaggacctttccagccatggcccggcctggaggagctgaatagcaagcagctgtcccggagactgcaggcaggaatcctgggcctgcccccttgtctgaatatcctgaggcgcaagcagccccagctgacatttttcaccatcgcactgcagagttgtcattatcagcgactgcctcctcatatcctgtgggctacaggcctttgaaa

SEQ ID NO: 217 - DNA, 686 bp, synthetic construct
  misc_feature 1..686: note = Synthetic
  source 1..686: mol_type = other DNA
agcagcgagaccggacctgtggcagtggacccaaccctgagaagacgcattgagccatttcattttcagtttaacaacgaccccagagcctaccggagaaagacctacctgtgctatgagctgaagcaggacggctccacctgggtgctggatcggacactgagaaacaagggccggcacgccgagatctgtttcctggacaagatcaattcctgggagaggctggatcccgcccagcactaccgcgtgacatggtatatgagctggtccccttgctctaactgtgcccagcaggtggtggatttcctgaaggagcacccacacgtgaatctgcggatctttgccgccagactgtactatcacgagcagaggcgctatcaggagggcctgcggagcctgaggggaagcggagtgcctgtggccgtgatgaccctgccagacttcgagcactgctgggagacatttgtggatcacggcggccggccattccagccatgggacggcctggaggagctgaactctaggagcctgtcccggagactgcaggcaggaatcctgggcctgcccccttgtctgaatatcctgaggcgcaagcagccccagctgaccttttttaccatcgcactgcagagttgtcactaccagagactgcctcctcatatcctgtgggctacaggcctttgaaa

SEQ ID NO: 218 - DNA, 852 bp, synthetic construct
  misc_feature 1..852: note = Synthetic
  source 1..852: mol_type = other DNA
tctggtggttctcccaagaagaagaggaaagtcggaagcggagctactaacttcagcctgctgaagcaggctggagacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtctggtggttctcccaagaagaagaggaaagtctaa

SEQ ID NO: 219 - DNA, 5409 bp, synthetic construct; identical to SEQ ID NO: 48
  misc_feature 1..5409: note = Synthetic
  source 1..5409: mol_type = other DNA

SEQ ID NO: 220 - protein, 166 aa, synthetic construct
  REGION 1..166: note = Synthetic
LGKEFCGAFYHPRSKSKQSCAIAKRGHDATLTRRYTNSKKHAEEFFLMDIDCQARHFWNKKWQITMYLTMQPCHSTDTGGTKEDQSCCEVMIKAKEKLGNVEIVIKPTHLCQVGKGKPEKGVRKLKTTGIELECMKEGDWKYYAQPEENPYDTSKTEELHNQQLEQ

SEQ ID NO: 221 - protein, 155 aa, synthetic construct
  REGION 1..155: note = Synthetic
CMATGTKCVGAKAQSPEGFVVNDCHAEVLCRRSFIHYLIKEIQKVFEPCEGDFHLQVKRDYHFYMYISQSPCGYGSEYETNGKAMEQSCSDKLCLLISPIYLTGIVICWDEARMKNALADRIEVSAIVWSFFKEVVQYKEIKRERYEQAKESLMK

SEQ ID NO: 222 - protein, 151 aa, synthetic construct
  REGION 1..151: note = Synthetic
SLGTGSKCIGEHKMSLEGWLVNDSHAEIIARRGFVKYLIGQLKSIFERSPIKGQYSIKNGGDASIFLSTKHSSCSDKMMRLIDKPVYLHSITVELYNQEAMERALVGRLCLSKELLGSIVWSDFKELVQNDLPVYYDWKQSSYQKARTLFN

SEQ ID NO: 223 - protein, 166 aa, synthetic construct
  REGION 1..166: note = Synthetic
SMGTGSKCVGQNKLSKDGDILNDSHAEVVARRGFLRYMYHQMEILFTVDAISKKFLQREGVSFIFFSSHTPCGDASIIVKENSMCNSCSDKLAKFLSKPIYVSHVIICPYSQSVMERALINRFEHSANQLSSISWQKFVSLLSDNLPIYNKAKRLCYFEMWEQTRA

SEQ ID NO: 224 - protein, 156 aa, synthetic construct
  REGION 1..156: note = Synthetic
SLCTGTKCLSGVELRSTGNKLSDSHAEILARRAFLRYLYHQIELIFYLEDNGIKLRDVSFHFFSSQTPCGDCSIIKLTGEDLSCSDKLAKLMIPTIKFESIVICPYSLESMQRGIFQRFDPSSIVWKTFLTIYDPQHPKYYHCKQYSYQNLWREKS

SEQ ID NO: 225 - protein, 157 aa, synthetic construct
  REGION 1..157: note = Synthetic
ALATGTKCLGDSELSKGGSRLSDSHAEVLTRRAFIRYLYDQIDLIFSRNDKNKIELNSNISFHFFSSQTPCGDCSIFKLETNSCSDKIAKLLIPPIKLESITISPFSLDAMERGLYKRFNNISIIWQSFLQIFDIRHPKYYDWKQWSYQNEWKQLKH

SEQ ID NO: 226 - protein, 159 aa, synthetic construct
  REGION 1..159: note = Synthetic
ALATGTKCLGDSELSESGSRLSDSHAEVLTRRAFIRYLYDQIDLIFSRNDDNEIELNNNISFHFFSSQTPCGDCSIFKEETTSDSCSDKMAKLMIPPIKLESITICPFSLDAMERGLYKRFNNISIVWRSFLQIFDIRHPKYYDWKQWSYQNEWKRLRR

SEQ ID NO: 227 - protein, 157 aa, synthetic construct
  REGION 1..157: note = Synthetic
ALATGTKCLGESELINDGSRLSDSHAEVLARRAFIRYLYDQIDLVFSWSHKNKIELNSNVSFHFFSSQTPCGDCSIFKHEIYSCSDKMAKLLIPPIKLKSITICPFSLDAMERGLYKRFNNTSIIWQTFLQIFDIRHPKYYDWKQWSYQSKWKQLKL

SEQ ID NO: 228 - protein, 158 aa, synthetic construct
  REGION 1..158: note = Synthetic
ALATGTKCLGESELTNSGSRLSDSHAEVLTRRAFIRYLYDQIDLVFKRNDKNKIELNSAISFHFFSSQTPCGDCSIFKEKTSNSCSDKIAKLMIPSIRLESITICPFSFDAMERGLYKRFNNSSIIWQTFLQVLDIKHPKYYDWKQWSYQNKWRQLQF

SEQ ID NO: 229 - protein, 163 aa, synthetic construct
  REGION 1..163: note = Synthetic
SLGTGTKCLGADQLSEKGDILNDSHAEVMARRGFLRFVMQQMTSIFEFNSESRKFSCKSGISFHFFTTHSPCGDASIYETSRSSCSDKMARLLERPIYLESVVITDFCKEAIERALWKRWDPDVVEGGGIVWATFAAVWKGADMDYVDVKNRSYREQWDALRV

SEQ ID NO: 230 - protein, 163 aa, synthetic construct
  REGION 1..163: note = Synthetic
SLGCGTKCIGQDKQCPKGYILNDSHAEIIARRAFLRYLYHELNKIFEWDHELVCYKLKKHYEYHFLCTHTPCGDACIGQLDTEVSCSDKLARLIHEPIYFTSFNFSEANVADIERAIFKRWQHKEFRNGLSWLCFLELLRIRKSLYGTCKQLAYQEAWQQLKS

SEQ ID NO: 231 - protein, 164 aa, synthetic construct
  REGION 1..164: note = Synthetic
SLATGTRCIGSSKLCARGFILNDCHAEVLARRGFLRYLNNEIMKIFSWQTAERCFTLNENLVFHFLSTQTPCGDACILESNIDTLSCSDKLSRLLNKPIYFTSFNFADAHLKSLQRALYQRWNGRSCKNSLIWLNFLNLLNLRQKLYAQCKALAYQLVWQQLKC

SEQ ID NO: 232 - protein, 162 aa, synthetic construct
  REGION 1..162: note = Synthetic
ALATGTRCIGASKLCAKGFVINDSHAEVLARRAFLRYLHNQLLKIFSWKSAAGCFTLNEQLEYHFLSTQTPCGDACIVDSEILSCSDKLSRLIDKPIYFSSLNFAEARLESLQRAVYQRWRGRNCSTSLIWLNFLDLLHLREKFYAECKALAYQLAWRQLKC

SEQ ID NO: 233 - protein, 163 aa, synthetic construct
  REGION 1..163: note = Synthetic
ALATGTRCIGASKLCNRGYILNDCHAEVLARRAFLRYLQNELLNVFIWKPSTGCFALNEQLEYHFLSTQTPCGDACIVDDACGVSCSDKLARVIDKPIYFISYNFTEANPESLERAIYRRWQGRECNSSLIWINFMDLLSVREKLYSELKALSYQLAWQQLKS

SEQ ID NO: 234 - protein, 164 aa, synthetic construct
  REGION 1..164: note = Synthetic
ALGCGTKCIGYTRHCPKGFILNDSHAEVLARRAFLRYLYHELEHIFQWAAKRGSFDLSAHVEYHFFSTQTPCGDACIVESVVVESCSDKLARLLSKPIYFSTLNFTEARQESVERAIYKRWQQGRDSKNGLVWLKFLNILHLREKFYASCKDLCYQQAWCQLKR

SEQ ID NO: 235 - protein, 161 aa, synthetic construct
  REGION 1..161: note = Synthetic
ALGLGTKCIPHTKLCENGFILNDSHAEVLARRAFQRYLYHELGQIFHWNCDSQCYDLDDHVEYHFLSTQTPCGDACIVEEDRSCSDKLSRLISKPIYFSTLNFADAHFESLNRAIYKRWEGRNFSNGLVWLTFLDCISLSEKFYATCKSWSYQEAWLQLKD

SEQ ID NO: 236 - protein, 161 aa, synthetic construct
  REGION 1..161: note = Synthetic
SLGCGTKCIGESKLCPKGLILNDSHAEVLARRGFLRFLYQELKQIFHWNSELSTFDMDEHVEFHFLSTQTPCGDACILEEQVSCSDKIARLISKPIYFSSLNFDDAQLESLERAIFKRFECRSFKNGLIWLTFLELVKLSEKFYASCKNLAYQFAWREIKE

SEQ ID NO: 237 - protein, 164 aa, synthetic construct
  REGION 1..164: note = Synthetic
AIGSGTTCLGESQMRPDGLILHDSHAEVIARRSFIRYLYHEIKSIFNKLIDGKCCLKPGISFHFYTSNTPCGDATIFVTVNQPVNTSCSDKILRLVSTPVYMRSIVVADIQALRRAFYGRMDLNSLDFSALSWRMFKEVMECELKSYRNCKLLAYFNTWSDVKD

SEQ ID NO: 238 - protein, 166 aa, synthetic construct
  REGION 1..166: note = Synthetic
AMGTGSKCIGRSKMRLEGDVLHDSHAEIIARRAFLRYLYHQLQMVFTTPGADCRCGLKPGVKFHLFTSHTPCGDASIFKGSKSTIDQSCSDKLARFMTEPVYFSTIVVCPYSSYAMYRGIVDRCAGIQDLAAIMWETFKQLLNERRPAYGDYKRAAHHTAKTCFLR

SEQ ID NO: 239 - protein, 166 aa, synthetic construct
  REGION 1..166: note = Synthetic
SLGTGTKCIGRSAMSMKGDVLNDSHAEVIARRGSVRYLTEQLLKVFCAGSEKGKWRLKAGVSFLFFTSQTPCGDASIFMSGSEPCETSCSDKLARYLQEALYFSAVLVSPYSHPALRRALHTRCSHVKDLAAISWHSFLKVVAAELPEYWDYKQAAYQLAWTQLRL

SEQ ID NO: 240 - protein, 166 aa, synthetic construct
  REGION 1..166: note = Synthetic
SLATGTKCIGRTALSPNGDVLNDSHAEVIARRGCVRYLIQELHRVFCRAEQQGKWKLKPGVSFHFFTSHTPCGDASIIMSDSQPCPGSCSDKMARYLQEALYFTSVVVCPYSQEVMHRALVARCLHVSDLAAISWHSFLSLVSSSLPSYWDYKRASYQQAWQQLHS

SEQ ID NO: 241 - protein, 166 aa, synthetic construct
  REGION 1..166: note = Synthetic
AMGTGTKCIGQAKLRKTGDVLQDSHAEIIAKRSFQRYLLHQLSLLFIPGTEKGKWMLRPEISFVFFTSHTPCGDASIIVISHELGHGSCSDKMARFLQQPIYLSAVVVCPFSQDAMERALYNRCHKVLSLAAVSWNTFRELVQKQRSEYWDYKAAAYQEAWNCLRQ

SEQ ID NO: 242 - protein, 166 aa, synthetic construct
  REGION 1..166: note = Synthetic
AMGTGTKCIGRNKMRKTGDILNDSHAEVVAKRSFQRYLLHQLWLIFSPGTETGKWKLKPNIIFIFFSSHTPCGDASIFISEPELSKNSCSDKLARFLQYPVYLSAVIVCPYSQEAMRRAVIERCQHVSSLAAISWHKFQRLMTEDLPDYWDYKQAAYQEAWRVLRS

SEQ ID NO: 243 - protein, 166 aa, synthetic construct
  REGION 1..166: note = Synthetic
ALGTGTKCIGLNKMRKTGDVLNDSHAEVVAKRSFQRYLLHQMRLIFIPGTETGKWKLKPNIIFIFFCSHTPCGDASIIIRETELSKSSCSDKLARFLQYPVYLSAVIVCPYSQEAMQRAVIERCRHISLLAAISWHEFQKLVTENLPDYWDYKEAAYQEAWKALRS

SEQ ID NO: 244 - protein, 166 aa, synthetic construct
  REGION 1..166: note = Synthetic
AMGTGTKCIGQNKMRKTGDILNDSHAETVAKRSFQRYLLHQMCLIFIPGTETGKWKLKPNIIFVFFSSHTPCGDASIIISEPELSKNSCSDKLARFLQYPVYLSAIIVCPYSQEAMRRAIIERCQHVSFLAAISWHAFQKLVTENLPDYWDYKEAAYQEAWKVLRS

SEQ ID NO: 245 - protein, 166 aa, synthetic construct
  REGION 1..166: note = Synthetic
AMGTGTKCIGQSKMRKDGDILNDSHAEVIAKRSFQRYLLHQIEAIFIPGTEVGKWKLKPDLSFVFFSSHTPCGDASIIMLDVEPCPRSCSDKLARLLQDPVYLAAVVICPYSPEAVRRAVIDRCRHVSPLAAISWGAFQKLVGDKQPAYWDYKEAAYQETWKALQR

SEQ ID NO: 246 - protein, 166 aa, synthetic construct
  REGION 1..166: note = Synthetic
SMGTGTKCIGLSKMRKNGDILNDSHAEVIAKRSFQRYLLHQLRSIFMPGTKTGMWKLKPDLHFVFFSSHTPCGDASIIMLELEPCSHSCSDKLARFLEEPVYLAALVICPYSPEAMKRAVIDRCQHVSSLAAISWNLFQNLVSSKRPEYWEYKEAAYQEAWKTLQK

SEQ ID NO: 247 - protein, 166 aa, synthetic construct
  REGION 1..166: note = Synthetic
SMGTGTKCIGQSKMRKSGDILNDSHAEIIAKRSFQRYLLYQLHMIFVPGTQRGLWKLRPDLSFVFFSSHTPCGDASIIMLEFEPCCSSCSDKVARFLEEPIYLSAVVICPYSQEAMQRALLGRCQNVSALAAISWRSFQKLLSDKWPDYQEHKEAAYQEAWRTLRK

SEQ ID NO: 248 - protein, 165 aa, synthetic construct
  REGION 1..165: note = Synthetic
SMGTGTKCIGQSKMRKSGEVLNDSHAEVIARRSFQRYLLHQPHLIFIPGTQRGLWKLRPDVFLFVSSHTPCGDASIIMLEFEPCYSSCSDKLARFLEEPVYLSAVVICPYSQEAMQRAPVRRCRMSPLYAAISWRSFQELLSDKWPDYQEYEEASYQEAWSALRK

SEQ ID NO: 249 - protein, 166 aa, synthetic construct
  REGION 1..166: note = Synthetic
SMGTGTKCIGQSKMRKSGDILNDSHAEIIARRSFQRYLLHQLHLIFVPGTRRGLWKLRPDLLFVFFSSHTPCGDASIIMLEFEPCCSSCSDKLARFLEEPIYLSAVVICPYSQEAMQRALTGRCHNISALAAISWRSFQKLLSDKWPDYQEYKEAAYQEAWSALRK

SEQ ID NO: 250 - protein, 166 aa, synthetic construct
  REGION 1..166: note = Synthetic
SMGTGTKCIGQSKMRKSGDILNDSHAEVIARRSFQRYLLHQLHLIFVPGTQRGLWKLRPDLSFVFFSSHTPCGDASIIMLEFEPCCSSCSDKLARLLEEPVYLSAVVICPYSQEAMHRALTGRCQNVSALAAISWRSFQKLLSDKWPDYHEYKEAAYQEAWSALRK

SEQ ID NO: 251 - protein, 166 aa, synthetic construct
  REGION 1..166: note = Synthetic
SMGTGTKCIGQSKMRESGDILNDSHAEIIARRSFQRYLLHQLHLIFVPGTQRGLWRLRPDLSFVFFSSHTPCGDASIIMLEFEPCCSSCSDKMARFLEKPIYLSAVVICPYSQEAMRRALTGRCEETLVLAAISWRSFQKLLSDEQPDYQEYKDAAYQEAWGALRR

SEQ ID NO: 252 - protein, 166 aa, synthetic construct
  REGION 1..166: note = Synthetic
SMGTGTKCIGQSKMRKSGDVLNDSHAEVIARRSFQRYLCHQLQLIFVLGTQKGQWKLRPGISFVFFSSHTPCGDASIIMLGFEPCYSSCSDKVARFLEEPIYLSAVVICPYSQEAMHRALIGRCQNISALAAISWRSFQKLLSDMWPNYQEYKQAAYQEAWSALRK

SEQ ID NO: 253 - protein, 166 aa, synthetic construct
  REGION 1..166: note = Synthetic
SMGTGTKCIGQSKMRKSGDILNDSHAEVIARRSFQRYLLHQLQLIFVPGTQKGLWKLRRDLFFVFFSSHTPCGDASIIMLEFEPCCSSCSDKMARFLEEPIYLSAVVICPYSQEAMQRALTGRCQNVSALAAISWRSFQKLLSDKWPDYQDYKEAAYQEAWSTLRK

SEQ ID NO: 254 - protein, 166 aa, synthetic construct
  REGION 1..166: note = Synthetic
SMGTGTKCIGQSKMRKSGDILNDSHAEVIARRSFQRYLLYQLQLIFVPGTQRGLWKLRPNLLFVFFSSHTPCGDASIIMLEFEPCCGSCSDKMARFLEEPIYLSAVVICPYSQEVMQRALIGRCQNVLALSAISWRSFQKLLSDKWPDYQEHKEAAYQEAWSALRK

SEQ ID NO: 255 - protein, 152 aa, synthetic construct
  REGION 1..152: note = Synthetic
ALGTGTKCLGGSQRSLAGDTINDCHAEVIARRTLLKLLYTDIGSNGRLRMRPEIRLHLYISQSPCGDACGVDSCSDKIARFLNEPVYISTITVSDLACIEALQRATFGRLCSVARNYSISWRSFRHLLTHFFPGYLECKKLAYLEAKEILLH

SEQ ID NO: 256 - protein, 160 aa, synthetic construct
  REGION 1..160: note = Synthetic
ALGTGTKCIGGSLLSPTGDVVNDSHAEIIARRSLLRYFYAEIERAFDTSCCGQTKYRMNPGWGLHLYITQLPCGVFSYPTLRELSCFNKITRILQPVYLSTLTVSVKTNYLEKAIYDCMERLHIKYSICWEVFVSLQSRLSLQYHGLKAMAYQSSLKMLR

SEQ ID NO: 257 - protein, 163 aa, synthetic construct
  REGION 1..163: note = Synthetic
SLGTGTKCIGRSRRSSKGDVVNDSHAEIIARRALLRYLYSEIQDMFESDGLGTKKLKMKHGWQLHLYISQLPCGVASPGSELAHDSMSCSDKIARFLEPIYIFSVTISIIEDEVMRAIHERVLPLSNKYSICWESFLSLSHNCVGEYRELKDKSYNLASETFK

SEQ ID NO: 258 - protein, 158 aa, synthetic construct
  REGION 1..158: note = Synthetic
AMGTGTKCLGRSLLRTCGDVVHDSHAEVIARRALIRFFYTQIQPFDPGCLNKGKYSLKKDWKLHMYISQLPCGDASLSVSPLSCSDKIARFLQPVYLSSITVFRLEDSLKRALYERMLPLSNEYSICWEVFLSLGPENLIGYRELKDGAYHLASKIFK

SEQ ID NO: 259 - protein, 162 aa, synthetic construct
  REGION 1..162: note = Synthetic
ALGTGTKCVSGSLLSPRGDIVNDSHAEVVARRALIRFFYSEIQRSIDSSCPGEVKYKLKSGCLLHLYISQLPCGYASTSSPLYKKISCSDKIARVLQPVYISTITVFSLADHLRRSLYERILPLSDEYSLCWELFLKETHGHKREYRELKNKAYYLMSKIFK

SEQ ID NO: 260 - protein, 164 aa, synthetic construct
  REGION 1..164: note = Synthetic
SLGTGNRCVTGDKLSMEGRTVNDSHAEIITRRAFLRYLYNQLQAILTQGTNGKLRLLPDVSLHLYISTAPCGDGAQFSRTDENESSCSDKVASFIEPMYLSSISLSLYHHGHLARAVCCRVSSELDNLLSINWSEYQQTCRLFDRTYHDAKLSAYYTAKQYLKC

SEQ ID NO: 261 - protein, 163 aa, synthetic construct
  REGION 1..163: note = Synthetic
SFGTGTRTASGDLLDLKGEVVFDSHAEIIARRGLKMFLYQELQVIFEANEDGKLRVKKSIKFHLYISTAPCGDGAQFSRLDNRLSSCSDKIGSFVEPVYLFSLSLSLHHHGHLSRAVCCRFHELGPYLSMNWNEFLSLSSVSGHTYAEAKSLAYQEAKGLLHK

SEQ ID NO: 262 - protein, 163 aa, synthetic construct
  REGION 1..163: note = Synthetic
SLGAGNRCVTGQRLSMEGKVVNDSHAEIIARRSLLRFFYAQLHAIFEKNNSRRLAVRQGVSFHLYISTAPCGDGALFTPRENTDLSCSDKICRFIEPVYLESLTLYLYDHGHLARAVCCRLQDLGAELSVNWESFKELCQRTDREYSQAKQMAFQNAKRELFE

SEQ ID NO: 263 - protein, 164 aa, synthetic construct
  REGION 1..164: note = Synthetic
SIGTGNRCITGPQLSLEGNTVNDSHAEIITRRGFIRFMYKQLQALFEPSPSGKLRLKDNITFHLYISTAPCGDGALFSPRDSNNASCTDKLCRLMDPVYLDSLTLLLYDHGHLARAMCCRLARDINSLLSVNWDNFKQVCADLGKQYGKAKTAAFQTAKKALIK

SEQ ID NO: 264 - protein, 163 aa, synthetic construct
  REGION 1..163: note = Synthetic
SMGTGNRCISGERLSQEGLVVNDSHAEIVTRRGFLRFLYKQLIEIFEPSENRKFRVKPDVTFHLYISTAPCGDGALFSKTDVSELSCSDKIARFVDPIYLTSITLYLYDHGHLSRAVCCRLSELDTLHSLSWASFKEACIKHQRDYYLVKTAAFQKAKKVLLE

SEQ ID NO: 265 - protein, 161 aa, synthetic construct
  REGION 1..161: note = Synthetic
SLGTGNRCVKGEELSLKGETVNDCHAEIISRRGFIRFLYSELLKIFEPAEENKLKIKPDITFHLYISTAPCGDGALFDCSEKSCSDKILRFLHPIYLKSITLYLYSHGHLTRAVCCRLARAFSQNSSVNWCLFRSLCQRCGRTYAQAKTSAFQLAKQQFFE

SEQ ID NO: 266 - protein, 161 aa, synthetic construct
  REGION 1..161: note = Synthetic
SIGTGNRCVKGEELSLKGETVNDCHAEIISRRGFIRFLYSELMKFLEVVSDGRMKIKTGVTFHLYISTAPCGDGALFDCSEKSCSDKILRFLHPVYLSSVTLYLYSQGHLARAICCRMSRAFQEGSSVNWQLFQQICTKTDRKYQEAKEAAFQKAKVHFVQ

SEQ ID NO: 267 - protein, 161 aa, synthetic construct
  REGION 1..161: note = Synthetic
SIGTGNRCVKGEELSLKGETVNDCHAEIISRRGFVRFLYSELMKIFEPAGKKRLKIKSNVTFHLYVSTAPCGDGALFDCSDKSCSDKILRFIEPVYLSSVTLYLYSQGHLTRAICCRMVRVLQKRSSVNWALFQQLCAKNKRKYSEAKEAAYQEAKQRFFS

SEQ ID NO: 268 - protein, 484 aa, synthetic construct
  REGION 1..484: note = Synthetic
  VARIANT 40: note = X can be any naturally occurring amino acid
SERILEGLYTHRGLYASNARGCYSVALLYSGLYGLUGLULEUSERLEUARGGLYGLUTHRVALASNASPCYSHISALAGLUILEVALSERARGARGGLYPHEILESERASNTHRILEXSERGLNLEUMETLYSILEPHEGLUGLUALAGLUGLYASPLEULEUARGVALARGPROGLYVALTHRPHEHISLEUTYRILESERTHRALAPROCYSGLYASPGLYALALEUPHEASPCYSSERASPLYSSERCYSSERASPLYSILELEUARGPHEMETGLUPROVALTYRLEUSERSERLEUTHRLEUTYRLEUPHESERGLNGLYHISLEUTHRARGALAILECYSCYSARGMETSERARGALAPHEGLNASNGLNSERSERVALASNTRPTHRLEUPHEGLNGLNLEUSERVALLEUARGGLYARGHISTYRSERASPVALLYSALATHRALATYRGLNTHRALALYSGLYGLNLEUPHEARG

SEQ ID NO: 269 - protein, 162 aa, synthetic construct
  REGION 1..162: note = Synthetic
SLGTGNRCVKGDSLSLKGETVNDCHAEIISRRGFIRFLYSELMKIFEPAKGGEKLQIKKTVSFHLYISTAPCGDGALFDCSDKSCSDKILRFLQPIYLKSVTLYLFSQGHLTRAICCRVTRAFEDGTSVNWLLFKKLCSFRYRRYGEAKKAAYETAKNYFKK

SEQ ID NO: 270 - protein, 164 aa, synthetic construct
  REGION 1..164: note = Synthetic
AMGTGNSCVMGKNIGTDGRTLNDCHAEVVVRRSLLRYLYRELKNIFEKHSDSLLVLKEGVSFHLYLTTAPCGDAATHITRDTRVQSCSDKLAKFIEPVYLSSVTFTNYDHGQLSRALCCRIDDHISADISLNWMRFKAMCTLARRQYNQAKMMCYQEAKRQLYD

SEQ ID NO: 271 - protein, 163 aa, synthetic construct
  REGION 1..163: note = Synthetic
ALGTGNSCYAGWLAFDGRLLHDCHALVVVRRTLQRFLYKQLLLIFCSSPGGSLSLRPGIFFHLYLGKVPEGAARTTFMYAPRNPSESDKMTRFIQPIYITSVVLHFYDRNTVSQVINKRLEGALIRKSSLNWSSFRKVAEKTGRSYHSVKLRAYQKVKKQVNC

SEQ ID NO: 272 - protein, 162 aa, synthetic construct
  REGION 1..162: note = Synthetic
ALGTGDICYEGWMEFNGRRVHDMHGMVVARRVLLRYFYKQLLMIFCPAGDGILALKPKYFLHLYLSRTPSGASENFHLAPVVQSGSDKLTRIILPVYITSVVLPYQDHTVLHEVVNDRVQLGPGNGLSLNWSMFRKVVQEMKREYHKGKVQAYQSAKLQMYT

SEQ ID NO: 273 - protein, 158 aa, synthetic construct
  REGION 1..158: note = Synthetic
ALGTGSSSCAGWLEFSGRQLHDCHGLVVARRALLRFLYRQLLLVLTPQPGPGFVLKPRIFIHFYISNTPKGAAHDIYPPAASASDKLSRFLPPLYATSLVLPCHDPPTLSSAIHTRLDRVSGSCLSLNWRAFRQAAQALGKSYEEAKAYQEARQQLSL

SEQ ID NO: 274 - protein, 158 aa, synthetic construct
  REGION 1..158: note = Synthetic
ALGTGSSSCAGWLEFSGRRLHDCHGLVIARRALLRFFFRQLLLVLTPQPGSGFALKPGVFLHLYVSNTPKGAAHDIYLASPSASDKLARFLPPLYATSLVLPCHDPPTLNRAIHSRLDSVLGSCLSLNWAAFRQVARALEKPYEAAKAYREARQQLSL

SEQ ID NO: 275 - protein, 158 aa, synthetic construct
  REGION 1..158: note = Synthetic
ALGTGNSCCAGWLEFSGRQLHDCHALVVARRALLRFLYRQLLLVLAPQPGPGFALKPRIFLHLYVSNTPKGAAHDIYSASTSASDKLARFLPPLYSTSLVLPCHDPSTLSRAIHSRLDGALGPVLSLNWRAFRQAAGALGKPYEAAKAYQEARRRLSL

SEQ ID NO: 276 - protein, 158 aa, synthetic construct
  REGION 1..158: note = Synthetic
ALGTGSSCCAGWLEFSGQQLHDCHGLVIARRALLRFLFRQLLLVLAPQPGPGFTLKPRVFLHLYISNTPKGAARDIYPTSPSASDKLARLVSPLYSTSLILSCHDPPTLSRAIHTRLDSVLGPCLSLNWRAFHQVARAVGKPYEAAKAYQEARRQLSL

SEQ ID NO: 277 - protein, 164 aa, synthetic construct
  REGION 1..164: note = Synthetic
ALGTGDFNYSQCICRDGRVVHDSHGVVMARRSLLRFLYRQLLLIFCIEPTSKLTIKPNANIHLYLNQLPKGAAQIKSQLRPQSQSASDKLTRFIQPVYISSVLIANCTDTRGLEIAVKQRVDDALTSRLSLNWSRFNLLAKESNREYHDAKIMSYQEAQCLLKS

SEQ ID NO: 278 - protein, 163 aa, synthetic construct
  REGION 1..163: note = Synthetic
AIGTGEYNYSQDIKPNGRVLHDTHAVVTARRSLLRYFYRQLLLIFCTEPTSNLTLKQNINICLYMNQLPKGSAQIKSQLRPHSESSSDKLTRFIQPVYISSILVGNCSDTRGLEIAIKQRVDDALTSKLSLNWSRFNLLAKEAKKYHAAKCMSYQEAKCKLKS

SEQ ID NO: 279 - protein, 165 aa, synthetic construct
  REGION 1..165: note = Synthetic
AVGTGEYNCSQCIKPNGRVLHDTHGVVTARRSLLRYFYRQLLLIFCTEPASDLTLKQDINIYLYMNQLPKGSAQIKSQLRPNSESSSDKLTRFIQPVYISNILVVGNCSDTRGLEIAIKQRVDDALTSRLSMNWSRFNLLAKEAKRDYHAAKCMSYQEAKTLLKS

SEQ ID NO: 280 - protein, 164 aa, synthetic construct
  REGION 1..164: note = Synthetic
ALGTGDCNYSNDYSPEGRVVHDSHAIVTARRSLLRYFYRHLLLLFCTAPDSKLTLKRNISIYLYMSQLPKGSAQIKTQFCPHSESASDKLTKFIEPVYINTILVGNCRSLKGLEIAIRQRIDDALTSKLSLNWSRFKCLAGRAKRTYHEAKVKSYQEAKKLLHS

SEQ ID NO: 281 - protein, 164 aa, synthetic construct
  REGION 1..164: note = Synthetic
ALGTGQCNYSQDYQPNGRVLHDSHAIVTARRSLLRYFYRHLMLIFCTAPGSKLVLKQNTNIFLYMNQLPKGSAQLKSQLHPQSESATDKLTKFIEPVYINSILVGNCKDTRGLEIAVKQRIDDALTVELSLNWSRFKSLAKEAERNYHKAKIQSYQEAKKLLHS

SEQ ID NO: 282 - protein, 164 aa, synthetic construct
  REGION 1..164: note = Synthetic
ALGTGECNYSRRFESCGRLLHDSHAVVTARRSLLRYFYRHLLLIFCTAPGSKLTLKRNITLSLYMNQLPKGTAQLESEVHPQSESAGDKLTKFIEPVYISNILVGSCKDTKGLDITIKQRLDDELTSKLSLNWSRFRMLAREAGRDYHEAKVKSYQEAKSLLQS

SEQ ID NO: 283 - protein, 163 aa, synthetic construct
  REGION 1..163: note = Synthetic
AIGTGDTNTNQHATANGRLLHDSHAVVTARRSLLRYLYRHLLLIFQLDLNSQLTLKRNITIHLYMNQLPKGSAQLPPRLHPCSNSASDKITQFIEPIYVGSILIASCSDVRGLEIAVKQRVEGITSKLSMNWSRFNLVAKEAEREYYEAKMSSYQEAKIVLKT

SEQ ID NO: 284 - protein, 163 aa, synthetic construct
  REGION 1..163: note = Synthetic
ALGTGSSNTKASPAPTGRILHDSHAVVTARRSLMRFLYRNLLLVFQQDETTKLSFKNHITLHLYLSQLPKGASQIPSQLRPLSNSATDKVMQFIEPIYMSSIFIGSCSDIRGMEMAVNQRVDGITSALSLNWSRFNLVAKESQREYREAKMMAYQEAKSMLKS

SEQ ID NO: 285 - protein, 163 aa, synthetic construct
  REGION 1..163: note = Synthetic
ALGTGNLNTKESLTPSGRILHDSHAVVTARRSLMRYLYRHLLLIFQQDQNTKLSLKSHITLHLYLNQLPKGAAQIPSHLRPLSNSATDKITQFIEPIYVSSILIESCSDTRGMEVAINQRVDGITSKLSLNWSRFNLVSKEAQREYREAKMMAYQEAKSVLKS

SEQ ID NO: 286 - protein, 163 aa, synthetic construct
  REGION 1..163: note = Synthetic
ALGTGGFNTRESISSDGRIVHDSHAVVTARRSLMRYLYRNLLMVYQHKSNSNLSLKTGISLHLYVNSLPKGAAVIPSKLYPLSSSTADKITQFIEPIYVQSILISCCTDVRGMEVSVCQRVEGVTSQLGINWHRFKLVAKEAQRHYREAKRMAYQEAKSVLRA

SEQ ID NO: 287 - protein, 162 aa, synthetic construct
  REGION 1..162: note = Synthetic
ALGTGNFNAKESASTGGRIVHDSHALVSARRSLMRFLYRHLLMVFEQSGSGLSLKSGITLHLYVNQLPKGAAQIPSQLRPLSNSATDKLTQFIEPVYVHSILVVGCSDVRGLKMSVSQRVEGITSQLGINWHRFKLVAKEARRLYREAKRMAYQEAKNVLRA

SEQ ID NO: 288 - protein, 160 aa, synthetic construct
  REGION 1..160: note = Synthetic
ALGTGTKTMTGDYISSIGTAVIDCHGEIISRRNLKRYFYAELQKIFERKDGQFALRDGIKFHLYINTTTCGDARVFNPNSDFESSCSDKVMRLIEPIYLSSISLGLYHRQHFPRAMFERIDDIGLDFSINWQLYLLGYATKIYLDAKNLSYQSVKNGLYE

SEQ ID NO: 289 - protein, 161 aa, synthetic construct
  REGION 1..161: note = Synthetic
CLATGTKCLSGNYLSLSGESLHDCHAEILTRRCLLKFFYKELMEIFVFSGDKKFKLAEDIAFHLYINTAPCGEARVFSFCDENSCSDKLTLFIEPTYLESISISVFNINHMKRTIYGRVENSIDKYCVNWKEYIKLCSLSTLIYDLLKNKNYIAAKNQILS

SEQ ID NO: 290 - protein, 164 aa, synthetic construct
  REGION 1..164: note = Synthetic
SLATGNKCIKATSLSFDGCAVNDCHAEILTRRGLVRWLYTQVQLLVEQGEEGGKLRLRKRFSLHLFISTAPCGDGRVYQFGSNKDNSCSDKVLKFLHPLLICLSIYLSSLALHFTRESCIARACYGRVARFKPVSANWTRFAALTPYKDVKRSAYSKTQFELIR

SEQ ID NO: 291 - protein, 156 aa, synthetic construct
  REGION 1..156: note = Synthetic
SLSTGNKGLRGDKVVSDGTALIDCHAEILARRGLLRFLYSEILKIFMKKGTLVLRPGISFHLFINTAPCGAGRIDNKAKSVKASCSDKLLRFVEPIYYSSIAVEKNNVERLQRAVFGRAASSSVNWELMVHVCAITQTYDELKAGCYETEKKAFIA

SEQ ID NO: 292 - protein, 155 aa, synthetic construct
  REGION 1..155: note = Synthetic
SLATGNKGLRGDQITSDGSALIDCHAEILARRGLLRFLYSEVLKIFETGKLALRQGISFHLFINTAPCGTARVDRKVRTAEASCSDKLLRFIEPVYYASIAVEQNNFVRMNKAVYTRASSGSMNWELMVKTCFLTKTYEEMKAGCYSAAKKSFIM

SEQ ID NO: 293 - protein, 155 aa, synthetic construct
  REGION 1..155: note = Synthetic
SLATGNKGLSGDKIRSDGSALIDCHAEILAKRGLMRFLYSEVLKIFLESGLILKLGITFHLFINTAPCGTARVDKKMRNPESSCSDKLLRFMNPIYYSSIAVEKSNFERMNRAVNDRAAGGSMNWELMKTVCTLAKIYEELKAGSYAATKKEFYA

SEQ ID NO: 294 - protein, 155 aa, synthetic construct
  REGION 1..155: note = Synthetic
SLSTGNKGLRGDKIVNDGSALIDCHAEILARRGLLRFLYSEVIKIFERGTLVLKKGILFHLFINTAPCGTARVDRRMKTAEASCSDKLLRFIEPIYYTSIAVEQNNFDRLNKAVFARAANGSMNWELMMATCKLTQTYDELKAGSYLNAKQSFIK

SEQ ID NO: 295 - protein, 155 aa, synthetic construct
  REGION 1..155: note = Synthetic
ALSTGNKGLRGDKIVNDGTALIDCHAEILARRGLLRFLYSEVLKIFTKGKLVLKPGISFHLFINTAPCGVARIDKKLKTSDNSCSDKLLRFIDPIYYSSIAVELNNADRLRKAVYSRAATSSMNWELMITICTLTKTYEELKAGSYAAAKKSFIT

SEQ ID NO: 296 - protein, 158 aa, synthetic construct
  REGION 1..158: note = Synthetic
SIGTGTKFISGEYISDKGYAVNDCHGEIIARRGLRKFLYNQLELIFELKPSGYGLKDQVEFHLYISTSPCGDARVFSPHEPEEKSCSDKICKFIEPVYFTSIILSLYRYNHMARAMYERVGPVEDHSLNWTKFLALWARQYDEAKLSAYQKAKASLMM

SEQ ID NO: 297 - protein, 162 aa, synthetic construct
  REGION 1..162: note = Synthetic
SVGTGTKCVGGEHISVKGAVLNDSHAEIISRRGLVRYFYSQLLLIFIRNEDGQFCLKPEIRFHLYISTSPCGDARIFSPHDNTSSCSDKVAKFIEPIYLHSIVVSLFHQTHLRRALYGRIENTLIGHSINWIKFHELLRKTGITYSDAKELVYKDAKTYMIA

SEQ ID NO: 298 - protein, 162 aa, synthetic construct
  REGION 1..162: note = Synthetic
SVTTGTKCISGEHISMNGCSLNDMHAEILSRRCLITYFYDQLELIFTQREDGKYKLKPGLDFHLYINTAPCGDARIFSPHEEASSCSDKICRFIEPIYLKSIVLSLMREAHMYRALCGRIENTIQGFAVVWKRFARLVGSVTEIYCDVKDAVYKAAKTNLYE

SEQ ID NO: 299 - protein, 161 aa, synthetic construct
  REGION 1..161: note = Synthetic
CVTTGTKCVSGEHLSVSGGAVNDCHAEVVARRCLCEYLYKQLELILEPAKKGFKLKQGIQFHLYINTAPCGDARIFSPHEESASCSDKIARFIEPIYFHSIVLSLLNPSHMYRAVCGRIENTIQGYSVNWRRFFNLLGTIEDVYLEAKLSVYSLAKRQLKD

SEQ ID NO: 300 - protein, 160 aa, synthetic construct
  REGION 1..160: note = Synthetic
SVSTGTKCVSGEHMSVNGAVLNDSHAEIVSRRCLLKYLYAQLDLIFVRNTDGQYKLKSGVHFHLYINTAPCGDARIFSPHETGASCSDKIARIIEPVYLHSIVLSLLHPEHMYRAVCGRIEKSIQGFGINWDKYGFLMKGMQYGETKADVYQTAKQELFS

SEQ ID NO: 301 - protein, 161 aa, synthetic construct
  REGION 1..161: note = Synthetic
SLATGTKCVSGEHMSVTGSVINDSHAEIIARRGLLDFFYTQLDLIFVAPTDGTYKLKDGIHFHLYINTAPCGDARVFSPHENMDASCSDKISRVIEPIYLHSIVLSLLHPAHMYRAICGRIENSIQGFSINWRRFAAVIEHARYGETKMCVYQQAKKELFA

SEQ ID NO: 302 - protein, 160 aa, synthetic construct
  REGION 1..160: note = Synthetic
SVSTGTKCINGEYMSDQGLAVNDCHAEVIGRRSLMRYLYFQLGKIFQEKEGGFMLKPNIHFHLYISTSPCGDSRIFSPHEQEASCSDKIARFIEPVYFDSLILSLYHGDHLSRAVYSRISNIENFAVNWKRFLQLCGLTGQSYADAKVTVYQTAKQQMYL

SEQ ID NO: 303 - protein, 161 aa, synthetic construct
  REGION 1..161: note = Synthetic
SLATGTKCINGEYMSDQGMALNDCHAEIVSRRSLLRYLYSQLDLVFEPKEDGKYRLKDNIQFHLYISTSPCGDARIFSPHETDASCSDKIARFVEPIYLSSIILSLYHGDHLSRAVYQRLGELEQFSVNWQCWNKLFGTTGRHYSEAKLLNYQAAKQEMVK

SEQ ID NO: 304 - protein, 161 aa, synthetic construct
  REGION 1..161: note = Synthetic
SVSTGTKCINGEYMSDRGLALNDCHAEIISRRSLLRFLYTQLELIFQKSERGFRLKENVQFHLYISTSPCGDARIFSPHELEAASCSDKIARFVEPIYFSSIILSLYHGDHLSRAMYQRISNIEDFSVNWCRWMRVHGHLLRSYHESKLAAYQAAKARLFT

SEQ ID NO: 305 - protein, 160 aa, synthetic construct
  REGION 1..160: note = Synthetic
SISTGTKCINGEYMSDRGLALNDCHAEIIARRSLLRFLYTQLELIFQKSERGFRLKENVQFHLYISTSPCGDARIFSPHELEASCSDKMARFVEPIYFSSIILSLYHGDHLSRAMYQRISNIEDFSVNWCRWMRVHGNLLRSYHESKLAAYQAAKACLFK

SEQ ID NO: 306 - protein, 160 aa, synthetic construct
  REGION 1..160: note = Synthetic
CVSTGTKCINGEYMSDRGLALNDCHAEIIARRSLIRYLYSQLEFIFVHYEKGYRLKDNVQFHLYISTSPCGDARIFSPHEVEASCSDKIARFMEPIYFSSIILSLYHADHLSRAMYQRIADIEDFSVNWSRWVRLHSSILSNYHEAKQAAYHSAKQALIK

SEQ ID NO: 307 - protein, 161 aa, synthetic construct
  REGION 1..161: note = Synthetic
CISTGTQCIDGEHLSEDGLTVNDCHAEVVARRCLVRFLYSQLELIFRRGECGRFRLKDKVQFHLYVSASPCGDACIFSPHDVETSCSDKMARFTEPLYFSSIIVSLYHAAHMSRAMYGRIGPLEGFSENWCRWMQLHSSFLRVYHQAKRAAYCSAKHTLYR

SEQ ID NO: 308 - protein, 161 aa, synthetic construct
  REGION 1..161: note = Synthetic
CVSTGTKCIGREYMSNRGLALNDCHAEIVARRSLIRFLYSQLEYIFMRCDNSQFRVKENVQFHLYISTSPCGDARIFSPHEVDASCSDKIARFTEPIYFSSIILSLYHADHLSRAMYQRITEIEEFSVNWSRWMNLHCSMLWIYHDTKQAAYHSAKETLFR

SEQ ID NO: 309 - protein, 160 aa, synthetic construct
  REGION 1..160: note = Synthetic
ALSSGTKCISGEHLSDQGLVVNDCHAEVVARRAFLHFLYTQLELIFVRLKEGYRLRENILFHLYVSTSPCGDARLHSPYETDFSCTDKIARFVEPVYLQSIVVSLHHTGHLARVMSHRMEGVGQFSVNWARWAQLYGTRTPSYCEAKLGAYQSVKQQLFK

SEQ ID NO: 310 - protein, 160 aa, synthetic construct
  REGION 1..160: note = Synthetic
ALSSGTKCINGEYLNDQGLVVNDCHAEIIVRRAFLHFLYTQLELIFIRLKEGYRLRENIMFHLYVSTSPCGDARLHSPYESDFSCTDKITRFIDPIYLQSIIVSLHHTGHLSRVMSHRIEDLGNFSLNWTRWARLYGTRIGNYCDAKLAHYQSVKQQMFK

SEQ ID NO: 311 - protein, 160 aa, synthetic construct
  REGION 1..160: note = Synthetic
ALSTGTKCINGEYLSDQGLVVNDCHAEVTARRALLRFLYSQLEFIFVRHKERYRLRDNIHFHMYISTSPCGDGRLNSPYESDHSCTDKITRFVEPVYLHSVTISLRHTGHLGRVLNQRLERLGPMSVNWTRWSRLYRTHASSYAEAKMAAYQSVKQQWFR

SEQ ID NO: 312 - protein, 160 aa, synthetic construct
  REGION 1..160: note = Synthetic
ALSTGTKCINGEYMSDQGLVVNDCHAEVTTRRALLRFLYSHLELKLVQQGRGFRLRVCVIYHLMAFVADCQTARFASPFFLSFSCTDKIARFVEPVYLSSLTVSLRHTGHLSRTLSQRLERLGPYSVNWTRWTRLYRACAPGYCEAKQAAYQTAKEHWVR

SEQ ID NO: 313 - protein, 160 aa, synthetic construct
  REGION 1..160: note = Synthetic
ALSSGTKCINGEYISDQGLVVNDCHAEITTRRALLRFLYSQLELIFVRHKDSLRLRENVLFHMYISTSPCGDARVNSPYESDFSCTDKISRFVEPVYLYSLTVSLRHTGHFSRMMNHRMEKAGPYSVNWARWVRLYRVRGASYCEAKQAAYQTVKLQWLK

SEQ ID NO: 314 - protein, 160 aa, synthetic construct
  REGION 1..160: note = Synthetic
AISTGTKCINGEYISDRGLVVNDCHAEIIARRAFVRFLYSQLELIFIRHKEAYRLRENILFHMYISTSPCGDARLNSPYEADFSCTDKITRLVEPVYLHSLIVSLHHTGHLSRIVTHRLDRMGHFSVNWARWAKLYRTQVPNYCEAKLAAYQTVKQQLFK

SEQ ID NO: 315 - protein, 160 aa, synthetic construct
  REGION 1..160: note = Synthetic
SLATGTKCLDLEDESDSGRILRDCHAEVISRRALVRFLYAQLELIFVRNKDNFRLQEGILFHMYVSSSPCGDARLNCPYEAAFSCTDKLAKLVEPIYLHSLTVTLCHTGHLARGMARRLTPVKHISLNWTRWLQLQRGPVSGFSACKMSAYQRALRRFGS

SEQ ID NO: 316 - protein, 162 aa, synthetic construct
  REGION 1..162: note = Synthetic
SLATGTKCLDLGGVSDSGCTLTDCHAEVVSRRALVRFLYSQLELIFIPNKSCGDFRLREGVHFHMYVSSSPCGDARLNCPYEAAFSCTDKLAKLVEPVYLQSLTVTLSHTGHLGRAMARRLAPIKHISVNWARWLRLQQGPVIGYCASKMSAYQRAVQQFSS

SEQ ID NO: 317 - protein, 162 aa, synthetic construct
  REGION 1..162: note = Synthetic
SLATGTKCLDLDRLNEDGWTLRDCHAEVLSRRALVRFFYSQLELIFVPDKDSTHFRLQEGIRFHMYVSLSPCGDARLNCPYEPAFSCTDKIAKLVEPIYLHSLTVRLSHTGHLGRAVTRRLARVKHVCVNWSCWLRLENTPVSGYQASKMGAYQKAMQQFSN

SEQ ID NO: 318 - protein, 161 aa, synthetic construct
  REGION 1..161: note = Synthetic
SLATGTKCRDSDGVGDREGTLSDCHAEVLSRRALVRFLYAQLELIFVSNTAGGFRLRDGVFFHMYASSSPCGDARLNCPHEAAFSCTDKIAKLVEPVYLHSLTVTLSHTGHLGRAVARRLAPVRRIKIIAARSRDVLTRHLNSYSGSKMAAYQRAMQQFTG

SEQ ID NO: 319 - protein, 160 aa, synthetic construct
  REGION 1..160: note = Synthetic
SLATGTKCRDRGSAADGAGSLSDCHAEVISRRALLRFLYSQLELVLTPKPGGYRLRDGLLFHMYVSCSPCGDARLNCPYEAAFSCTDKLAKLIEPVYLQSLTVTLSHTGHLSRALTRRLAPVRRTSSNWARWRRLQQAHLDGYAGWKAAAYQRVMQTFSS

SEQ ID NO: 320 - protein, 160 aa, synthetic construct
  REGION 1..160: note = Synthetic
SLATGTRCPGRAGARDRAGTLSDCHAEVISRRALLRFLYWQLELIFTPNSGGCRLRDGVLFHMFVSSSPCGDARLNCPYEAAFSCTDKLAKLIEPVYLHSLTVTLSHTGHLSRAMARRLAPVRQISTNWVRWQQLQQPQLNGYSGWKGAAYRRMMQNFIS

SEQ ID NO: 321 - protein, 143 aa, synthetic construct
  REGION 1..143: note = Synthetic
KYPSRTYYKQTCIVCNITTGENTLFKLAKSDGKHAEELLLEELEKLVPKDSLTITIFMNDTPCSLAGHDCAGKFVQYLKTAKVDLTLYVTSLCESKKKDIHKGLAGLKQCTVKSPNRDAWQEFIMDLNDWKKYEKQEKDGIEK

SEQ ID NO: 322 - protein, 137 aa, synthetic construct
  REGION 1..137: note = Synthetic
NLDHQDWDKAKSCLICKIDDNITVLEKTEGTHAEEFLLQELEKKVRNVTIFMNNSPCSTPNHTCADKLLKYLDASENVRMTMYVTRLYMIARDGHHAGLQRLKQHACEINAFNKNWEELITKMEFKEYNVRSRKEED

SEQ ID NO: 323 - protein, 123 aa, synthetic construct
  REGION 1..123: note = Synthetic
YGNSKEQLFMLAKIPELFPESRHAEENLILGFSGIEQFFPEQIRKIDIFLSHSPCSEQGVKCSGSLYLNGCDQKLIAFFKKGNDLDRSLFLENSNVQVRYHRQFDSAVENFIIQAPELKDWKN

SEQ ID NO: 324 - protein, 126 aa, synthetic construct
  REGION 1..126: note = Synthetic
ISNELQVSAPKKKQRLFMLDNVPAVFPESNHAEENLIRRFPDVSQFFPGQIINITIFLSHSPCCAEGEKHSDARPINGCNKKLSVFFEKGNALDASLFVNTKFKVQYLYRFNHTVEPFIRQAPLLH

SEQ ID NO: 325 - protein, 126 aa, synthetic construct
  REGION 1..126: note = Synthetic
IGNELSKQDTPRKHQLFMIAKVPQLFPQHAQHAEENLIRGFPSINKFYPNQISKIDIFLTHSPCSEEGGKHSSQNSVNGCDKKLTVFFKKGNSADVQLFNRNTKVKVYYNHKFDSATDEFIKEAPI

SEQ ID NO: 326 - protein, 128 aa, synthetic construct
  REGION 1..128: note = Synthetic
IGNELKNSDLDRKQKLFMIGGVPQQFPRHSQHAEENLIRNFPNIKKFFPNQINTVEIFLTHSPCSSNGQKYSAQCYTNGCDKKLSAFFKKENYFDQQLFNRKVKVRIHYNHQFDPSINHFIKEAPILK

SEQ ID NO: 327 - protein, 149 aa, synthetic construct
  REGION 1..149: note = Synthetic
ERFLNAPLVRKPNTILMLRVQSEEMYLCYTRGYANSLHAEQFMMEDPELLRLLQPVRTLLMTLLMTQQPCHENAHASCTLRLLRWCKRERGIDLSIRIARVFRAADESARDGLRLLMRAPVEVAMLTCEDWQHCDERLVAFRPWHEMED

SEQ ID NO: 328 - protein, 154 aa, synthetic construct
  REGION 1..154: note = Synthetic
FFHCSPTPEARKTKGVVVAALRRGDLRSNSSHAEEYVVRDEELVRAVAPEDAGTLTLYQRLQPCHGSSDNRGSCSDAVAGLHRELLGVSLRVAVSYTYRAHVRGFREGIRVFAADGVTLEALNAEDWALCDEDYAAAFGGVFTAAVAHRRAMDE

SEQ ID NO: 329 - protein, 115 aa, synthetic construct
  REGION 1..115: note = Synthetic
PPPNIFLAQTREREAETGLRLLLYMTYQPCHHSGGRPKEALGTCSESLRDFYIKELGVALELVLADVYKATEELHREGMRMLLAEGITMRAMQPADWDLCDPEWAKRGAGPFSKH

SEQ ID NO: 330 - protein, 144 aa, synthetic construct
  REGION 1..144: note = Synthetic
CGKVVVAVALEHWRAGSSENVHAESFVIADETILSAVVAELTLYLSYQPCHHSGGRGGGEARQSCTEELIAYHERELNVPLSVVVADLYKVMEELMREGIRRLMSPGMSMRATGEEDWALCDQSYERRGGSAFGPHVALRAKLD

SEQ ID NO: 331 - protein, 152 aa, synthetic construct
  REGION 1..152: note = Synthetic
RGRRDGPVIVARVLGQSADLYVAYARGRSENVHAEEFMLADPQLLALLDAGGARILRLYMSYQPCHHSGGRKTPEDARKSCSERLRAFYEAEMAISLELVVADLYKAMEDLMRQGILLMVAPGVTMRATDENDWALCDPAYATRGGSAFSRH

SEQ ID NO: 332 - protein, 156 aa, synthetic construct
  REGION 1..156: note = Synthetic
VDTRDKTKAQVIVARVLGQNADLYVAYARGRSENVHAEEFMLADPQLLALLDASGARILRLYMSYQPCHHSGGRKTPEDARKSCSERLRAFYEAEMAISLELVVADLYKAMEDLMRQGILLLVAPGVTMRATDENDWALCDPAYETRGGSAFSRHE

SEQ ID NO: 333 - protein, 164 aa, synthetic construct
  REGION 1..164: note = Synthetic
SCKSFDERGASTMVACLRTTNGTYQEYRATDNDDRHPEEIFHKDMLQARTYVLPLTHIIVYLPTSPCFHQDCEPQCDVLDACAEQLAIVYRQAKKTDLKMSVKFLASYIGDLYKQGITMMMNAGIDVEPLGMRDWIELVHNDSTYYFSWKGLLTSYIRQSEIYI

SEQ ID NO: 334 - protein, 160 aa, synthetic construct
  REGION 1..160: note = Synthetic
RKLQYRGSSALVAQLTETATYRAFVVSEKTIHVEQKFYHQLMDAANFTMPLREVILYLPTSPCFHQDCDPLCDVLDACAEALSICYKQIRREELQMTVKFLASYVGDLYKQGIMSMMEAGLTVEPLNMKDWISLVSTAGTYYRDWENSLADYVMQTQLYI

SEQ ID NO: 335 - protein, 148 aa, synthetic construct
  REGION 1..148: note = Synthetic
AQLTETATYRAFVVSEKTVHVEQKFYHHLMDAANFTMPLRELILYLPTSPCFHQDCDPLCDVLDACAEALSICYKQIQREELQMTVKFLASYVGDLYKQGIMSMMEAGITVEPLNMKDWISLVSTAGTYYRDWENSLADYVMQTQLYI

SEQ ID NO: 336 - protein, 165 aa, synthetic construct
  REGION 1..165: note = Synthetic
LNEQVQKWEHRGASVMVAKLSDQQVYADYFVGDGKPQHVEQIFYTELMNADQCQMATLRQICLFMPTSPCFHQDCEPQCDVLDACAETLAIVYKQLQNSDLQMTVKFLASYVGDLYKQGILCMLQAGISVEPLNRKDWQALVEPDHTRAWDSLLTNYAMQSQFYI

SEQ ID NO: 337 - protein, 165 aa, synthetic construct
  REGION 1..165: note = Synthetic
LNEQVQKWEHRGASVMVAKLSDQQVYADYFVGDSKPQHVEQIFYTELMNADQCQMTTLRQICLFMPTSPCFHQDCEPQCDVLDACAETLAIAYKQLQNSDLQMTVKFLASYVGDLYKQGILCMLQAGISVEPLNRKDWQALVESDHSRSWDSLLTNYAMQSQFYI

SEQ ID NO: 338 - protein, 165 aa, synthetic construct
  REGION 1..165: note = Synthetic
PNEQVQKWEHRGASVMVAKLSDQHVYADYFVAETKPQHVEQIFYAELINADQCRMTTLRQICLFMPTSPCFHQDCEPQCDVLDACAETLAIAYKQLQNPDLQMTVKFLASYVGDLYKQGILCMLQAGISVEPLSRKDWYALVESDHTRGWDSLLTNYAMQSQFYI

SEQ ID NO: 339 - protein, 163 aa, synthetic construct
  REGION 1..163: note = Synthetic
VPKHVQNCEHKHESGLIVTLGDGYIYGDFFHDSNPHVEEQLVAAIYDLSKYKVDLYEIVIFVSKSPCFHQDCDPKCEVVDACAKLLGLLLSKVRKVDVRMTVKFLYPHLGDLYKQGILSMLQSGIKVEPLLMKDWSAVMDWAGEYLQWGSHLDRAVAQSQAFI

SEQ ID NO: 340 - protein, 163 aa, synthetic construct
  REGION 1..163: note = Synthetic
FHVPKHVQSCEHKHESGLIVTLGEDYIYGDFYHESSPHVEEQLVAAIYDLSKYKVDLYEIVIFVSKSPCFHQDCDPKCEVVDACAKLLGLLLSKVRKVDVKMTVKFLYPHLGDLYKQGILCMLQAGIKVEPLLMKDWSAIMDWTGEYLQWNNHLDKAVAQSQS

SEQ ID NO: 341 - protein, 165 aa, synthetic construct
  REGION 1..165: note = Synthetic
FHIPKHVQSCEQKHESGLIVTLGDDYVYGDFYHESNPHVEEQLVAAIYDLSKYKVDLYEIMIYVSKSPCFHQDCDPKCEVVDACAKLLGLLLSKVRKVDIRMTVKFLYPHLGDLYKQGILCMLQAGIKVEPLLMKDWSAVMDWSAEYLQWNNHLDKAVAQSQAFI

SEQ ID NO: 342 - protein, 164 aa, synthetic construct
  REGION 1..164: note = Synthetic
FHVPKHVQSCEQKHESGLIVTLGEDYIYGDFYHESSPHVEEQLVAAIYDLSKYKVDLYEMVIYVSKSPCFHQDCDPKCEVVDACAKLLGLLLSKVRKVDVKMTVKFLYPHLGDLYKQGILCMLQTGIKVEPLLMKDWSAVMDWSGEYLQWNNHLDKAVAQSQSF

SEQ ID NO: 343 - protein, 162 aa, synthetic construct
  REGION 1..162: note = Synthetic
IPKHVQSCEHKNESGLIVTLGDDYIYGDFYHESSPHVEEQLVAAIYDLSKYKVDLNEVVIYVSKSPCFHQDCDPKCEVVDACAKLLGLLLSKIRKVDVRMTVKFLYPHLGDLYKQGILCMLQAGIKVEPLLMKDWSTVMDWSGEYLQWNNHLDKAVAQSQSF

SEQ ID NO: 344 - protein, 162 aa, synthetic construct
  REGION 1..162: note = Synthetic
VPKHVQNCEHKNESGLIVTLGHDYVYGDFYHESSLHVEEQLAAAIYDLSKYKVDLFEAVIYVSKSPCFHQDCDPKCEVVDACAKLLGLLLSKIRKVDVKMTVKFLYPHLGDLYKQGILCMLQAGIKVEPLLLKDWSAVMDWTGEYLQWNNHLDKAVAQSQSF

SEQ ID NO: 345 - protein, 164 aa, synthetic construct
  REGION 1..164: note = Synthetic
FHLPKHVQSCEHKNETGLILTLGDDYIYGDFYHESSLHVEEQLVAAIYDLSKYKVDLFEAVIYVSKSPCFHQDCDPKCEVVDACAKLLGLLLSKIRKVDVKMTVKFLYPHLGDLYKQGILCMLQAGIKVEPLLLKDWSAVMDWTGEYLQWNSHLDKAVAQSQSF

SEQ ID NO: 346 - protein, 162 aa, synthetic construct
  REGION 1..162: note = Synthetic
VPKHVQNCEHKHESGLIVTLGEDYIYGDFYHESGPHVEEQLVAAIYDLSKYKIELYEIVIFVSKSPCFHQDCDPKCEVVDACAKLLGLLLSKVRKVDVKMTVKFLYPHLGDLYKQGILCMLQAGIKVEPLLMKDWSAIMDWAGEYLQWNNHLDKAVAQSQSF

SEQ ID NO: 347 - protein, 163 aa, synthetic construct
  REGION 1..163: note = Synthetic
VPKHVQNCEHKHESGLIVTLGEDYVYGDFYHESGPHVEEQLVAAIYDLSKYTVDLHEIQIFVSKSPCFHQDCEPKCEVVDACAKLLGLLLSKVRKVDVKMTVKFLYPHLGDLYKQGILCMLQAGIKVEPLLMKDWCAIMDWSGDYLQWNNHLDKAVAQSQLFI

SEQ ID NO: 348 - protein, 145 aa, synthetic construct
  REGION 1..145: note = Synthetic
IPKHVQNCDHKHESGLIVSLGDDYIYGDFYHESGPHVEEQLVANIYDLSRYKVDLHEIVIFVSKSPCFHQDCDPKCEVVDACAKLLGLLLSKIRKVDVKMTVKFLYTHLGDLYKQGILCMLQAGIKVGFFSVLQKAVAQSQLFIN

SEQ ID NO: 349 - protein, 163 aa, synthetic construct
  REGION 1..163: note = Synthetic
IPKHVQNCDHKHESGLIVSLGDDYVYGDFYHESGPHVEEQLVANIYDLSRYKVDLHEVVIFVSKSPCFHQDCDPKCEVVDACAKLLGLLLSKIRKVDVKMTVKFLYTHLGDLYKQGILCMLQAGIKVEPLLMKDWCAIMDWSGDYLQWNNHLDKAVAQSQLFI

SEQ ID NO: 350 - protein, 163 aa, synthetic construct
  REGION 1..163: note = Synthetic
VPKHVQNCDHKHESGLIVSLGENYIYGDFYHESGPHVEEQLVANIYDLTKYNVELHEIVIFVSKSPCFHQDCDPKCEVVDACAKLLGLLLSKVRKVDVKMTVKFLYPHLGDLYKQGILCMLQAGIKVEPLLMKDWCAIMDWSGDYLQWNNHLDKAVAQSQLFI

SEQ ID NO: 351 - protein, 158 aa, synthetic construct
  REGION 1..158: note = Synthetic
FSVPKHVQNCDHKHESEDYIYGDFYHESGPHVEEQLVANIYDLTKYNVELHEIVIFVSKSPCFHQDCEPKCEVVDACAKLLGLLLSKVRKVDVKMTVKFLYPHLGDLYKQGILCMLQAGIKVEPLLMKDWCAIMDWSGDYLQWNNHLDKAVAQSQLFI

SEQ ID NO: 352 - protein, 140 aa, synthetic construct
  REGION 1..140: note = Synthetic
VPKHVQNCDHKHESGLIVSLGEDYIYGDFYHESGPHVEEQLVANIYDLSKYNVELHEIAIFVSKSPCFHQDCDPKCEVVDACAKLLGLLLSKVRKVDVKMTVKFLYPHLGDLYKQGILCMLQAGIKVCGHFFVCFAYLFV

SEQ ID NO: 353 - protein, 161 aa, synthetic construct
  REGION 1..161: note = Synthetic
FAVTKNTKQCGQKNETAAIVTLGGGNIEFHHESSLHPEEQLFTALMELSEYQVNLEEVVLYCSKSPCYHQDCNPLCEVIDACAKLLVLLLYKVRTVDVNLTVRFLYPHLGDLYKQAIMYMLQHGINVEPLLMSDWSAIVEWGCNYLDWNEHLDQAVARGQS

SEQ ID NO: 354 - protein, 164 aa, synthetic construct
  REGION 1..164: note = Synthetic
FLVSRSTQQCEHKHESAIVVTLGEDYVYIEYAHESGLHCEEQLMNALEELAMYTVNLYEVVVYTSRSPCFHQNCEPRCAVIDACSKLLSLFLLKLRRVDLRMTVRFLFPHLGDLYKQGILCMLQHGIKVEPLLMKDWSAIMDWSGDYLAWNQYLDHAVAKSQSF

SEQ ID NO: 355 - protein, 162 aa, synthetic construct
  REGION 1..162: note = Synthetic
ISRTTQHCEHKHESAIVVTLGEDYVYVEYLHESSLHCEEQLLYALEELAKYAINLYEVLVYTSRSPCFHQNCEPRCAVIDACSMLLSLFLFKLRRVDLRMTVRFLFPHLGDLYKQGILCMLQHGIKVEPLLMKDWSGIMDWAGDYLTWNQHLDQAVAKSQSF

SEQ ID NO: 356 - protein, 162 aa, synthetic construct
  REGION 1..162: note = Synthetic
VSRTTQHCEHKHESATVVTLGEDYVYVEYLHESSPHCEEQLLFALEELAKYAINLYEILVYTSRSPCFHQNCEPRCAVIDACSMLLSLFLFKLRRVDLRLTVRFLFPHLGDLYKQGILCMLQHGIKVEPLLMKDWSGIMDWAGDYLAWNQHLDQAVAKSQSF

SEQ ID NO: 357 - protein, 148 aa, synthetic construct
  REGION 1..148: note = Synthetic
SRQGQQCEQKHESVILVTLGEDYVYVEFCHESSRHCEEQLAIALHELVNRSPCFHQDCEPRCDVIDACSKLLALLLTKVRKADIRMTVRFLFPHLGDLYKQGILCMLQHGIKVEPLLMRDWSAIMDWAGDYLGWNEHLDQAVAKSQSF

SEQ ID NO: 358 - protein, 161 aa, synthetic construct
  REGION 1..161: note = Synthetic
SRQGQQCEQKHESVILVTLGEDYVYVEFCHESNRHCEEQLAIALHELVKYKINLYEILIYSSRSPCFHQDCEPRCDVIDACSKLLALLLTKVRKADIRMTVRFLFPHLGDLYKQGILCMLQHGIKVEPLLMRDWSAIMDWAGDYLGWNEHLDQAVAKSQSF

SEQ ID NO: 359 - protein, 146 aa, synthetic construct
  REGION 1..146: note = Synthetic
WTAFYINGRPKLKKCITLCHVVFNETATAEQWEISYSHGPHAEIKVLRNIKARELCLGYTRIVTLFLSYSPCANCANFIIEFSRTRPQCTVYIRFTCLFRHPEEIHRDGLRRLNAPGISLGVFTVYEWRRLAEAGMPFRPWDSKWK

SEQ ID NO: 360 - protein, 150 aa, synthetic construct
  REGION 1..150: note = Synthetic
FYGKFNNTRKNGRNMLCFSLEGENKPWKWGYAHNSKHAENIVLREVASYKPFLNHFYISYGPCSNCCDKILDFLQKFEKIKIMIKISRLYKDESSVFQNSIKKLHQMGVSVQVMNRGDFEQCFKGFVQGDFQPWPALEPTSEKCAANLEA

SEQ ID NO: 361 - protein, 151 aa, synthetic construct
  REGION 1..151: note = Synthetic
FEAEFNNTVNSFRTLLCFSLQQENKTWNWGYAHNNDSHAEILVLREIEKYEKADHEIRQRVTLYVTCSPCNRCCTKILEFFQRFQRFDMDIKISKIYDLDSLQDLKQLGVSLKVMDSSDFKECFDLFVHTAEFEPWPGLEEKTKQLNAVFL

SEQ ID NO: 362 - protein, 158 aa, synthetic construct
  REGION 1..158: note = Synthetic
FYQEFCNTLRTCRTLLCFSLCQSTKIWDWGYAYNKGSHAESMVLEEIKTFQNQDKTLKYTLTLYMSFSPCNECCYRLATYAKLERRIKINVMFSKLYFPEHRKIQKGLQYLECAGVSLKVMEKQDFVTCFYLFVTEHAFQEWHCLDDMTKQYSSTLQA

SEQ ID NO: 363 - protein, 155 aa, synthetic construct
  REGION 1..155: note = Synthetic
FEENFNNTVLVRKTLILFSFKKRSSIWKWGYAYNDGQHAECLVLSNIEQFENQIKGKYKMTFFMSHTPCHKCSDKIVSFLASRKGLSMKIKASRPYFLNEGRKGLYLLKRIGVLLKMMDRTDFEECFYLFVHPLTFTPWSDLDEQSKKNMDDLAA

SEQ ID NO: 364 - protein, 158 aa, synthetic construct
  REGION 1..158: note = Synthetic
FYEHFCNIKTPHQTLLCFSLKEDDKTWKWGHAYNNGYHAEILVLREIEDYANILNATKYTITWFLSYSPCHCCCDEITNFLMKFQKIEFNIKAARPYYFDNDKNQKGLKILKKLGVLIKMMDFTDYEECLYLFVDPCKFTAWPDLEVQSIANKMIFHH

SEQ ID NO: 365 - protein, 154 aa, synthetic construct
  REGION 1..154: note = Synthetic
FHRNFSSTKASHQTLLCFGLKEEDKTWKWGHAYNNGCHAEILVLREIENYTNIPNAAKYNITWFLSYSPCHSCCDKIINFFMNSQKIEFNIKAAKPYQFSNDQNPKGIKMLNKLGILFKMMEYSDFEECFYLFVDPCKFTAWPDLEAQSIANIT

SEQ ID NO: 366 - protein, 104 aa, synthetic construct
  REGION 1..104: note = Synthetic
LFEAGGYLDAVTCENIRCIILYSSYSPCNEVPHCCVSKIYNFSLKYPEITLCIYFSQLYHTQCPSAREALRSLWSPRVTLQRPPGELWPSFVCGGSLSHPLRTS

SEQ ID NO: 367 - protein, 103 aa, synthetic construct
  REGION 1..103: note = Synthetic
FEVGGYLDAVVYKNIRCIILYANYSPCNEAYHCCISKIYNFLLKYPEITLCIYFSRLYHTEFPTAQEALRSLSSPRVTLQRLPAGTQHYFVYGGPLYHPSRTL

SEQ ID NO: 368 - protein, 102 aa, synthetic construct
  REGION 1..102: note = Synthetic
EVGGYLDTVVYENIRCIIVYSNYSPCNEAYHCCVSKIYNFLLRYPEITLCIYFSKLYHTEFPTSREALRSLSSPQVTLQRLPGGAWHYFVYGGSLYHPSRTL

SEQ ID NO: 369 - protein, 104 aa, synthetic construct
  REGION 1..104: note = Synthetic
EVGGYLDAVAYENIRWIILYSNYSPCNEAHHCCVSKIYNFLLKYPEIRLCIYFSQLYHTEFPTAREALRSLSSPRVILQRLPGGMWHYFVYGGSLYHLSRTLQQ

SEQ ID NO: 370 - protein, 103 aa, synthetic construct
  REGION 1..103: note = Synthetic
EVGGYLDAVAYENIRCIILYSNYSPCNEAYHYCISKIYNFLLKYPEITLCIYFSQFYHTEFPTAREALRSLSSPRVTLQRLPGGARRYFVYGGALYHPSRTLR

SEQ ID NO: 371 - protein, 104 aa, synthetic construct
  REGION 1..104: note = Synthetic
LFEMGGYLDAVAYENIRRIILYSTYSPCNEAYHCCISKIYNFLLKYPEITLCIYFSQLYHTESPTAREALRSLSSPRVTLQRLPAGAQRYFVYGGSLYHPSRTL

SEQ ID NO: 372 - protein, 103 aa, synthetic construct
  REGION 1..103: note = Synthetic
EVGGYLDAVAYENMGCIILYSNYSPCNEAYHCCVSKIYNFLLKYPEITLCIYFSQLYHTEFPTAREALRSLSSPRVTLQRLPGGAWRYFVYGGSLYHPSRTLR

SEQ ID NO: 373 - protein, 99 aa, synthetic construct
  REGION 1..99: note = Synthetic
GYLDAVACENIGCIILYSNYSPCNEAHHFCISKIYNFLLKYPEIALCIYFSQLYHTEFPSAREALRSLSSPRVTLQRLPGGTWRYFVYGGSLYHPPRTL

SEQ ID NO: 374 - protein, 159 aa, synthetic construct
  REGION 1..159: note = Synthetic
RAFGFPCRAPQTHHLLFYELKSFSGTVVQKGHATSEDNHPESMLFEADGYLDAVAYRNIGCITLYSNYSPCNEAYHCCVSKIYNFLLKYPEITLCLYFSQPYHTEFPTARQALHSLASPQVTLQPLPTGTWCHFVYGGSLQHPPGTLRQNPHQINNFRG

SEQ ID NO: 375 - protein, 160 aa, synthetic construct
  REGION 1..160: note = Synthetic
LRAFGFPCRAAQTHHLLFYELRSFSGTVVQKGHASSEDSHPESMLFEADGYLDAVAYGNTGCITLYSNYSPCNEAYHCCISKIYNFLLKHPETTLCLYFSQPYHIEFPTARQALRSLASPWVTLQPLPMGTRCHFVYGGSLQHPPGTVGQNPHQINNLRG

SEQ ID NO: 376 - protein, 77 aa, synthetic construct
  REGION 1..77: note = Synthetic
LFEVGSYLDAVAHENVGCIILYSNFLPCNEAYHCCISNIYNFLLKYPEITLCIYFSQLYHTQFCATRKALQSFSSEN

SEQ ID NO: 377 - protein, 101 aa, synthetic construct
  REGION 1..101: note = Synthetic
VGGYLDAVAYGNIGCIILYSNYSPCNEAYHCCISKIYNFLLKYQEIILRIYFSQLYHTEFPTACRALWSLSSPRVTLQRLPGGAWHYFVHGGSFYHPSRTL

SEQ ID NO: 378 - protein, 104 aa, synthetic construct
  REGION 1..104: note = Synthetic
LFEAGGYLDSVTYESIGHIILYSNYSPCNEADHCCISKIYNFLIKHPEVTLCIYFSQLYHTEFPTAREALRSLSSPHVTLYPLSGGIRHYFVYGRSLYHPSRTL

SEQ ID NO: 379 - protein, 102 aa, synthetic construct
  REGION 1..102: note = Synthetic
LFEAGGYLDAVTYEDIGYIILYSNYSPCNEADHCCLSKIYSFLTKYPQVTLCIYFSKLYHIELPTAHEVLKSLSSPHVTLHPLCGGIWHYFVHGGSLYHPSR

SEQ ID NO: 380 - protein, 160 aa, synthetic construct
  REGION 1..160: note = Synthetic
FHQAFGFPYTHQNKHLLFYEVRHFSGKLMQKGHATNEDIHPESMLFETGGYLDSINWENVMYIILYSNYSPCNEAEQCCISKIYHFLMKYPGITLCIYFSQLYHTELPISCEALQSLASPQVTLNPLCGGLWHSFVTSQACYHPIRALRQNAQQINNITG

SEQ ID NO: 381 - protein, 160 aa, synthetic construct
  REGION 1..160: note = Synthetic
FHKAFGFPYMPQNKHLIFYELRSFSGTLVQKGHATNKNIHPESMLFEMGGYLDALDYDSIRYIILYSNYSPCNEAEHCCISKIYNFLTKYPDITLCIYFSQLYHMEFPVSCEALRSLASPHVTLNPLCGGVWHSFVSGEALYHPARALQQNSYKINNITG

SEQ ID NO: 382 - protein, 153 aa, synthetic construct
  REGION 1..153: note = Synthetic
FYEAFGFPYTPRNKHLLFYELRNISGTLIQKGQATNLNLHPESTLFDLDGYLDSIIYDNISYITLYSNYSPCNESNHYCIGKMYDFLISYPSTRLDIYFSQLYHTDFPESREALRSLAGPRVTISPISGGTWLSFVNGQALYNPTRAFKHNAY

SEQ ID NO: 383 - protein, 160 aa, synthetic construct
  REGION 1..160: note = Synthetic
FYEAFGFPYTPENKQLIFYEVKDFSGTNIQKGQVTNSNIHAESILFEDSGYLDALHHGSVGYITLYANYTPCNEYGHYCISKMYNFLLKYEDTRLDIYFSQLYHVESPAARQALRSLASPRVTVNPLSEGIWQTFAKGLSFYEPARASSSNASTIHLITG

SEQ ID NO: 384 - protein, 157 aa, synthetic construct
  REGION 1..157: note = Synthetic
FYQTFGFPHMPQPEHLTFYELKTFSGAPVQKGQATSQSIHPESMLFEEGGYLDSVYDDSIGHIILFSNYTPCNEAGHCCISQMFDFLMTYPDITLSIYFSQLYHTEFPASRKALRSLASPRVSVNPISGGIWHAFVSGGALRHPSRALGHNAYEINA

SEQ ID NO: 385 - protein, 154 aa, synthetic construct
  REGION 1..154: note = Synthetic
FYQIFGFPYSPQTQHLTFYELKTSSGSLVQKGHASGEDTHPESMLFEMDSYLEAVNNDNIEHVFLYSNYCPCNEANHCCISKMYNFLMRYPAISLNIYFSQLYHTEFPVSREALRSLASPQVTVNPMSGGIWHYFVSDEALHQPARTLRHNAYE

SEQ ID NO: 386 - protein, 144 aa, synthetic construct
  REGION 1..144: note = Synthetic
FHQIFRFPYTPSTKHLTFYELKTFSGSLVQKGHASNGHTHPESMLFEMNGYLDSANNSSIKHIILYSNNSPCNEANHCCISKMYNFLTMYPDVTLSVYFSHLYHTGFPASREALRSLASPQVTLSPISGGIWHCFVTGGAAFQP

SEQ ID NO: 387 - protein, 154 aa, synthetic construct
  REGION 1..154: note = Synthetic
FHQVFGFPHTPPTKHLTFYELKTSSGTLVQKGHASNGDTHPESMLFEMNGYLDSASNSTIKHIILYLNNFPCNEANHCCISKMYNFLMMYPDITLSIYFSQLYHTKFPTSREALRSLASPQVTLSPISGMIWHSFVMGGPAFQPGRALRHNAYE

SEQ ID NO: 388 - protein, 160 aa, synthetic construct
  REGION 1..160: note = Synthetic
FHQTFGFPWTPQTKHLTFYELRSSSKNLIQKGLASNGHNHPEAMLFEKNGYLDAVHNSNIRHIILYSNNSPCNEAKHCCISKMYNFLMNYPEVTLSVFFSQLYHTEFPTSRKALQSLASPQVTLSPICGGLWHAFVSNGSVPQPGRILRYNTYEINSIIA

SEQ ID NO: 389 - protein, 160 aa, synthetic construct
  REGION 1..160: note = Synthetic
FHQTFGFPWTPQTKHLTFYELRSSSGNLIQKGLASNGHTHPESMLFERDGYLDSLHDSNIRHIILYSNNSPCDEANHCCISKMYNFLMNYPEVTLSVFFSQLYHTEFPTSREALRGLASPQVTLSAISGGIWQSFVSGGLAVRPGRTLRYNAYEINCITE

SEQ ID NO: 390 - protein, 158 aa, synthetic construct
  REGION 1..158: note = Synthetic
FYQIFGFPYMPQTKHLTFYELKTTSGSLVQKGHASSGNTHPESMLFELNGYFDSANNDSIRHIILYSNNSPCNEANHCCISKMYNFLIMYPHVTLSIYFSQLYHTEFPASREALRSLASPQVTLSPISGGIWHSFVSGGPVFQPGRALRHNAYEINAI

SEQ ID NO: 391 - protein, 157 aa, synthetic construct
  REGION 1..157: note = Synthetic
FYQIFGFPYTPQTKHLTFYELKTSTGGLVQKGHASSTSTHPESMLFETNGYFDWANNGGIRHIILYSNHSPCNEADHCCISKMYNFLTTYPDVTLSIYFSQLYHTDFPASREALRSLASPKVTLSPISGGIWHFFVSGGSVFQPGRALRHNAYEINA

SEQ ID NO: 392 - protein, 160 aa, synthetic construct
  REGION 1..160: note = Synthetic
FYQLFGFPYTPQTKHLAFYELRPSSGSLVQKGHASSGDTHPESMLFERNGYFDSANNNGIRHIILYSNNSPCNEANHCCISKMYNFLRMYPDVTLSIYFSQLYHTEFPASREALRSLASPQVTLSPISGGIWHSFVSGGWLFQPGRALRHNAYEINAITG

SEQ ID NO: 393 - protein, 127 aa, synthetic construct
  REGION 1..127: note = Synthetic
FYQLFGFPYVPQTKHLTFYELKTSPGSLVQKGHENSGDTHAESMLSEMNGYFDSANHQGIRHIILYSNNSPCDEANHCCTSKMYNFLTMYPDVTLSIYFSQLYHTEFPASREALRSLASPQVTVSPP

SEQ ID NO: 394 - protein, 158 aa, synthetic construct
  REGION 1..158: note = Synthetic
FYQIFGFPYISQTKHLTFYELKTSSGSLVQKGHASRGDTHPESMLFEMNGYLDSANNDGIRHIILYSSNSPCNEANHCCISKMYNFLKVYPDVTLSVYFSQLYHTEFPASREALRSLASPQVTLSPISGGIWHSFVSGGSVFQPGRALRQNAYEINAI

SEQ ID NO: 395 - protein, 159 aa, synthetic construct
  REGION 1..159: note = Synthetic
FYQIFGFPYTPPTKHLTFYELKTSSGSLVQKGHASSGDTHPESMLFEVNGYFDSANNDCIRHIILYSSNSPCNEANHCCISKMYTFLAKYPDITLSVYFSQLYHTEFPASREALRSLASPQVTLSPISGGIWYSFVSGGAVFQPGRALRHNAHEINAIT

SEQ ID NO: 396 - protein, 160 aa, synthetic construct
  REGION 1..160: note = Synthetic
FYQIFGFPYTPPTKHLTFYELKTSSGSLVQKGHASSGDTHPESMLFEMNGYFDSASNDGMRHIILYSSNSPCNEANHCCISKMYTFLAKYPDITLSIYFSQLYHTEFPASREALRSLASPRVTLSPISGGMWYSFVSGGAVFQPGRALRHNAHEINAITG

SEQ ID NO: 397 - protein, 160 aa, synthetic construct
  REGION 1..160: note = Synthetic
FYQIFGFPYTPQTKHLTFYELKTSSGSLVQKGHASSGNTHPESMLFEMNGYFDSANNNAIRHIILYSNNSPCNEANHCCISKMYNFLILYPDITLSIYFSQLYHTEFPASREALRSLASPQVTLSPISGGMWHSFVSGGSVFQPGRALRHNAYEINAITG

SEQ ID NO: 398 - protein, 160 aa, synthetic construct
  REGION 1..160: note = Synthetic
FHQIFGFPYVPQTKHLTFYELKTSSGSLVQKGHASSENTHPESMLFEMNGYFDAAKNNAIRHIILYSNNSPCNEANHCCISKMYNFLIMYPDVTLSIYFSQLYHTEFPASREALRSLASPQVTLSPISGGIWHSFVSGGPVFQPRRALRHNAYEINRITG

SEQ ID NO: 399 - protein, 159 aa, synthetic construct
  REGION 1..159: note = Synthetic
FYQIFGFPYAPQTKHLTFYELKTSSGSLVQKGHASSGNTHPESMLFEMNGYLDSANNNAIRHIILYSSNSPCNEADHCCISKMYNFLIMYPDVTLSIYFSQLYHTEFPASREALRSLASPQVILSPISGSIWHSFVSGGSVFPPGRAQRHNAYEINAIT

SEQ ID NO: 400 - protein, 160 aa, synthetic construct
  REGION 1..160: note = Synthetic
FYQIFGFPYVPQTKHLTFYELKTSSGSLVQKGHASSGNTHPESMLFEMNGYFDSANNNTIRHIILYSNNSPCNEANHCCIGKMYNFLITYPDVTLSIYFSQLYHTEFPASREALRSLASPRVTLSPISGGIWYSFVSGGSVFQPGRALRHNAYEINAITG

SEQ ID NO: 401 - protein, 160 aa, synthetic construct
  REGION 1..160: note = Synthetic
FYQIFGFPYTPQTKHLTFYELKTSSGSLVQKGHASSGQIHPESMLFEMNGYLDLANNGGIRHIVLYSNHSPCNEAHHCCISKMYNFLITHPDITLSIYFSQLYHTEFPTSREALRSLASPRVTLSPISGGIWHSFVSGGSVFQPGRALRHNAYEINAITG

SEQ ID NO: 402 - protein, 157 aa, synthetic construct
  REGION 1..157: note = Synthetic
FCQIFGFPYTPQTKHLTFYELKTSSGNLVQKGHASSGYIHPESMLFEMNGYLDSANNDNIRHIILYCNNSPCNEANHCCISKVYNFLITYPGITLSIYFSQLYHTEFPASREALRSLASPRVVLSPISRGIWHSFVSGGSVFQPGRALRHNAYEINA

SEQ ID NO: 403 - protein, 154 aa, synthetic construct
  REGION 1..154: note = Synthetic
FCQIFGFPYTHQTKHFTFYELKTPSGSLVQKGHASSGYIHPESMLFEMNGYLDSASNDSIRHIILYSSNSPCNEANHCCISKMYNFLITYPGVTLSIYFSQLYHTEFPASREALRSLASPQVILSPISGGIWHSFVSGGSVFQPRRALRHNAYE

SEQ ID NO: 404 - protein, 154 aa, synthetic construct
  REGION 1..154: note = Synthetic
FCQIFGFPYAPQTKHLTFYEVKTSSGSLVQKGHASSGDIHPESMLFEMNGYLDSANNDSIRHIILYSSNSPCNEANHCCISKMYNFLITYPGVTLSIYFSQLYHTEFPASREALRSLASPRVVLSPISGGIWHSFISGGSIFQPGRALRHNAYE

SEQ ID NO: 405 - protein, 154 aa, synthetic construct
  REGION 1..154: note = Synthetic
FCQIFGFPYTPQTKHLTFYELKTSSGSLVQKGHASSGYIHPESMLFEMNGYLDSANNDSIRHIILYSNNSPCNEANHCCISKMYNFLITYPGITLSIYFSQLYHTEFPASREALRSLASPRVVLSPISGGIWHSFISGGSVFQPGRALRHNAYE

SEQ ID NO: 406 - protein, 151 aa, synthetic construct
  REGION 1..151: note = Synthetic
DPSVLRRVQYLLYEVKWSNSRKLTQCCHSTRTEHAEIYFLEDVFHRQRYDPSDHCSLTWYMSWSPCGECCKAIRDFLKEQPNVNLVIYVARIYCHEEENNRQGLRSLVNIVTIRIMDLPVYSYCWRTFVCDEDKDEDYWPRHFAPWIMLYS

SEQ ID NO: 407 - protein, 117 aa, synthetic construct
  REGION 1..117: note = Synthetic
HAEIYFLKDVFNRQRNDPSDHCSLTWYMTWSPCGECCKAIRDFLKEQPNVNLVIYVARIYCHEEENNRQGLRSLVNIVTIRIMDLPVYSYCWRTFVCDEDNFPGSPEGSHHKSMLLT

SEQ ID NO: 408 - protein, 73 aa, synthetic construct
  REGION 1..73: note = Synthetic
ITLYLSWSPCRNCCYEMQYFLKKHPNVNICIYLARLYYTEDEEICKALKDLSEKVIISVMKIEDYIYCWKTFV

SEQ ID NO: 409 - protein, 62 aa, synthetic construct
  REGION 1..62: note = Synthetic
ITWYLSWSPCVNCCYKIRDFLNRHSYVTIRIYVARLCYRGFHRNRKGLRNLVSLRVTVNVME

SEQ ID NO: 410 - protein, 64 aa, synthetic construct
  REGION 1..64: note = Synthetic
ITWYLSWSPCVNCCNEILDFLERHENVNIDIHVARLYFKDSKRTHRALKELARSTVSINVMNME

SEQ ID NO: 411 - protein, 151 aa, synthetic construct
  REGION 1..151: note = Synthetic
QRNFDPREFPECTLLLYEIHWDNNTSRNWCTNKGLHAEENFLQIFNEKIDIRQDTPCSITWFLSWSPCYPCSQAIIKFLEAHPNVSLEIKAARLYMHQIDCNKEGLRNLGRNRVSIMNLPDYRHCWTTFVVPRNEDYWPQDFLATNYSREL

SEQ ID NO: 412 - protein, 62 aa, synthetic construct
  REGION 1..62: note = Synthetic
ITWYLSWSPCMTCCYIIRNFLVRHPNVNIEIHVARLYNTRWAGTRRGLRELARLRVTIDVME

SEQ ID NO: 413 - protein, 154 aa, synthetic construct
  REGION 1..154: note = Synthetic
FDPRTYPSETYLLCELQWGGSGRFWIHWARNDEDSHVEHYFLEQIFEPRSYSVCDITWYLSWSPCANCCDIIQEFLEEQHNVNLDIRVARVYNEHIRENRAALRQLANFQAAIRAMDVEDYMYCWDTFLQQGGYFDFTAGSFRSAVERTRLRLE

SEQ ID NO: 414 - protein, 75 aa, synthetic construct
  REGION 1..75: note = Synthetic
DMTWYLSWSPCGECCDIIQDFLEEQPNVNINIRVARLYYTDRASNRKGLRELASSPVTLEIMDAEDYNYCWETFI

SEQ ID NO: 415 - protein, 64 aa, synthetic construct
  REGION 1..64: note = Synthetic
ITWYLSWSPCANCCYEIVDFLQRHSYVNIKIFVARLYYIDRERNRQGLRDLMNSAVTIDVMDIE

SEQ ID NO: 416 - protein, 64 aa, synthetic construct
  REGION 1..64: note = Synthetic
ITWYLSWSPCANCCYRIVQFLMKHSYVSIDIRVARLYFIEDETTRQGLEELVSCAVRLTVMDTE

SEQ ID NO: 417 - protein, 69 aa, synthetic construct
  REGION 1..69: note = Synthetic
ITWYLSWSPCACCCCKIQDFLKMNSYVNTDIDVAQLYGNYQEQNCQGLKNLKSLAVTIAVMRIEDKISC

SEQ ID NO: 418 - protein, 64 aa, synthetic construct
  REGION 1..64: note = Synthetic
ITWYLSWSPCANCCRKIRNFLKKHSYVYIDIYVARLYYIDDEENRRGLRNLQSLDVTIAVMEIE

SEQ ID NO: 419 - protein, 64 aa, synthetic construct
  REGION 1..64: note = Synthetic
ITWYLSWSPCADCCCKIVNFLKKHSYVNMRIYVARLYYPEYETNRRGLKNLRNLAVPIAVMEIE

SEQ ID NO: 420 - protein, 159 aa, synthetic construct
  REGION 1..159: note = Synthetic
FEKNYKPIDGTKEAHLLCEIKWGKYGKPWLHWCQNQRNIHAEDYFMNNIFKAKKHPVHCYVTWYLSWSPCADCASKIVKFLEERPYLKLTIYVAQLYYHTEEENRKGLRLLRSKKVIIRVMDISDYNYCWKVFVSNQNGNEDYWPLQFDPWVKENYSRL

SEQ ID NO: 421 - protein, 64 aa, synthetic construct
  REGION 1..64: note = Synthetic
ITWYLSWSPCARCCYKILDFLKEHSYVNLHIYVARLYCIEDEKTRRGLKKLNSLEVTIAVMEEE

SEQ ID NO: 422 - protein, 64 aa, synthetic construct
  REGION 1..64: note = Synthetic
ITWYLSWSPCAKCCYEILNFLKKHPNVNIDIYVARLYDIEKEKTRQGLKNLVRLPVTIAVMEME

SEQ ID NO: 423 - protein, 64 aa, synthetic construct
  REGION 1..64: note = Synthetic
ITWYLSRSPCAKCCYEILDFLNKHSNVNIDIYIAQLYKIKNEENCQGLRNLVSLAVTIAVMEIE

SEQ ID NO: 424 - protein, 137 aa, synthetic construct
  REGION 1..137: note = Synthetic
SELTWGGRPYKHWYENTEHCHAEIHFLENFSSKNGSCIITWYLSWSPCAECSARIADFMKENTNVKLNIHVARLYLHDDKHTRQGLRYLMKMKVTIQIMTIPDYKYCWNTFLEDDGEDESDDYGGYAGVHEDEDESD

SEQ ID NO: 425 - protein, 64 aa, synthetic construct
  REGION 1..64: note = Synthetic
ITWYLSWSPCAECCLKILNFLEENSNVNIDIHIARLYRIQDERNRQGLRELVSSEVTIAVMGIE

SEQ ID NO: 426 - protein, 64 aa, synthetic construct
  REGION 1..64: note = Synthetic
ITWYLSWSPCAKCCRKILNFLKMHSNVKIDIYVARLYYIEDEKNRQGLKKLVSLAVKIAVMEIE

SEQ ID NO: 427 - protein, 74 aa, synthetic construct
  REGION 1..74: note = Synthetic
VTWYLSWSPCVNCCRKILNFLKKHSNVNIDMHVARLYYIEDERIRQGLKNLVSLAVTIAVMEIEDYTYCWKNFI

SEQ ID NO: 428 - protein, 64 aa, synthetic construct
  REGION 1..64: note = Synthetic
ITWYLSWSPCAHCCRKILNFLKRHSDVNIHIYIARLYYIENEEIRQGLKNLVSLEVKIAVMETE

SEQ ID NO: 429 - protein, 64 aa, synthetic construct
  REGION 1..64: note = Synthetic
ITWYLSWSPCVNCCRKILKFLKQHSYVNIKIYVARLYYIDDDEIRQNLKNLVSLVVTIAVMDIE

SEQ ID NO: 430 - protein, 471 aa, synthetic construct
  REGION 1..471: note = Synthetic
  VARIANT 11: note = X can be any naturally occurring amino acid
  VARIANT 105: note = X can be any naturally occurring amino acid
  VARIANT 123: note = X can be any naturally occurring amino acid
LEULYSTYRHISPHEASPPROARGGLUVALXARGASPTHRTYRLEULEUCYSILELEUARGTRPGLYGLUTHRGLYTHRPROTRPSERHISTRPVALLYSASNARGTYRHISALAGLUVALTYRPHELEUGLULYSILEPHEGLNTHRARGLYSSERSERLYSASNILEASNCYSSERILETHRTRPTYRLEUSERTRPSERPROCYSALALYSCYSCYSARGLYSILELEUASNPHELEULYSLYSHISSERTYRVALSERILELYSILEHISVALALAARGLEUPHEARGILEASPASPLYSGLUTHRXGLNASNLEULYSASNLEUGLYSERLEUVALVALTHRVALSERVALMETGLUXGLUASPTYRTHRASNCYSTRPLYSTHRPHEILEARGGLYHISALAASPGLYASPSERTRPILEASPASPLEULYSSERGLUILEARGLYSASNARGLEULYSPHEGLN

SEQ ID NO: 431 - protein, 64 aa, synthetic construct
  REGION 1..64: note = Synthetic
ITWYLSWSPCADCCHKILKFLKKHSNVNIDIHVARVYYAEDEKVRQGLKNLVSLAVTIAVMETK

SEQ ID NO: 432 - protein, 64 aa, synthetic construct
  REGION 1..64: note = Synthetic
ITWYLSWSPCADCCRKILNFLKKNSNVNIDIYVARLYYTEDEKIRQGLQNLVSLAVTIAVMETE

SEQ ID NO: 433 - protein, 64 aa, synthetic construct
  REGION 1..64: note = Synthetic
ITWYLSWSPCADCCHKILNFLKRHSNVNIDIYVARLYYIEDEEIRQCLKNLVSLAVTIAVMKIE

SEQ ID NO: 434 - protein, 68 aa, synthetic construct
  REGION 1..68: note = Synthetic
ITWYLSWSPCANCCRKILRFLRKHSNVNIDIHVARLYYIEDENIRQGLKSLVNLAVTIAVMEIEGKVF

SEQ ID NO: 435 - protein, 64 aa, synthetic construct
  REGION 1..64: note = Synthetic
ITWYLSWSPCADCCHKILNFLKKHSNVNIIIYVARLYYKEDEKIRQGLKNLVNLAITIAVMEIE

SEQ ID NO: 436 - protein, 64 aa, synthetic construct
  REGION 1..64: note = Synthetic
ITWYLSWSPCEECCCKILNFLKKHSNVSICIYVARLYHIEDEKIRQGLKNLVNFTVTVAVMGIE

SEQ ID NO: 437 - protein, 155 aa, synthetic construct
  REGION 1..155: note = Synthetic
FEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRL

SEQ ID NO: 438 - protein, 157 aa, synthetic construct
  REGION 1..157: note = Synthetic
FEVFFDPRELRKEACLLYEIQWGTSHKIWRNSGKNTTKHVELNFIEKFTSERHFCSSVSCSIIWFLSWSPCWECSKAIREFLSQRPTVTLVIFVSRLFQHMDQQNRQGLRDLINSGVTIQIMRASEYDHCWRNFVNYPPGKEAHWPRYPPLWMKLYA

SEQ ID NO: 439 - protein, 158 aa, synthetic construct
  REGION 1..158: note = Synthetic
IFDPRELRKEACLLYEIKWGTGHKIWRHSGKNTTRHVEVNFIEKITSERQFCSSTSCSIIWFLSWSPCWECSKAITEFLRQRPGVTLVIYVARLYHHMDEQNRQGLRDLVKSGVTVQIMTTPEYDYCWRNFVNYPPGKDTHCPIYPPLLMKLYALELH

SEQ ID NO: 440 - protein, 160 aa, synthetic construct
  REGION 1..160: note = Synthetic
FEAFFDPRELRKEACLLYEIKWGMSHNIWRYSGKNTTKHVEINFIEKFTSERHLRPSISCSIVWFLSWSPCWECSKAIREFLSQHPNVTLVIYVARLFQHMDRLNRQGLRDLINSGVTIQIMRTSEYDHCWRNFVNYPPGKEAHWPRYSLLWMKLYALEL

SEQ ID NO: 441 - protein, 152 aa, synthetic construct
  REGION 1..152: note = Synthetic
VFFDPRELRKETCLLYELQWGRSRDTWRHTGKNTTNHVERNFLAKITSERHFHPSVHCSIVWFLSWSPCWECSEAIREFLDQHPSVTLVIYVARLFQHMDPQNRQGLRDLVNHGVTIQIMGAPEYDYCWRNFVNYPPGKEAHWPRFPPVWMT

SEQ ID NO: 442 - protein, 153 aa, synthetic construct
  REGION 1..153: note = Synthetic
FDPRNFCKEAYLLYEIQWGNSRDVWRHSGKNTTKHVERNFIEKIASERHFRPSISCSISWYLSWSPCWECSKAIREFLNQHPNVTLVIYIARLFQHMDPQNRQGLKDLFHSGVTIQVMRDPEYDYCWRNFVNYPQGKEAHWPRYPPLWMNLYA

SEQ ID NO: 443 - protein, 155 aa, synthetic construct
  REGION 1..155: note = Synthetic
FEVFFDPRELRKEACLLYEIKWGTSHRIWRNSGRNTANHVELNFIEKFTSERHFCPSVSCSITWFLSWSPCWECSKAIRGFLSQHPSVTLVIYVSRLFWHLDQQNRQGLRDLVNSGVTVQIMRVPEYDHCWRNFVNYPPGEEDHWPRYPVVWMKL

SEQ ID NO: 444 - protein, 161 aa, synthetic construct
  REGION 1..161: note = Synthetic
FEGFFDPRELRKETCLLYEIQWGTSHKTWRNSGKNTTNHVEINFMEKFAAERQYCPSIRCSITWFLSWSPCWECSNAIRGFLSQHPSVTLVIYVARLFWHTDPQNRQGLRDLINSGVTIQIMTVPEYDHCWRNFVNYPPGKEDHWPRYPVLWMKLYALELH

SEQ ID NO: 445 - protein, 151 aa, synthetic construct
  REGION 1..151: note = Synthetic
FEVFFNPGVLRKETCLLYEIQWGTSRKIWRNSSKNTTNHVEVNFIEKFTAERHFCPSISCSITWFLSWSPCWECSKAIREFLSQHPNMTLVIYTARLFQHMDQQNRQGLRDLINSGVTIQMMTVSDSLTCWLHFLSHRVYVILMKKCGNLV

SEQ ID NO: 446 - protein, 152 aa, synthetic construct
  REGION 1..152: note = Synthetic
FEVFFDPQELRKEACLLYEIKWGASSKTWRSSGKNTTNHVEVNFLEKLTSEGRLGPSTCCSITWFLSWSPCWECSTAIREFLSQHPGVTLVIFVARLFQHMDRRNRQGLKDLVTSGVTVQVMSVSEYCYCWENFVNYPPGKAAQWPRYPPRW

SEQ ID NO: 447 - protein, 157 aa, synthetic construct
  REGION 1..157: note = Synthetic
FEASFDPRQLQKEACLLSEVRWGASPRTWRESGLNTTSHVEINFIEKFTSGRSLRPAIRCSVTWFLSWSPCWECARAIREFLHQHPNVSLVIYVARLYWHVDEQNRQGLRDLVTSGVRVQIMSDSEYSHCWRNFVNFPPGQEAGWPRFPPMWTTLYA

SEQ ID NO: 448 - protein, 157 aa, synthetic construct
  REGION 1..157: note = Synthetic
FEAYFDPRQLRKEACMLSEVRWGASPRTWRESSLNTTSHVEINFIEKFTSGRSLRPAVRCSMTWFLSWSPCWECARAIREFLHQHPNVSLVIYVARLYWHVDEQNRQGLRDLVTSGVRVQIMSDSEYRHCWRNFVNFPPGQEAGWPRFPPMWTTLYA

SEQ ID NO: 449 - protein, 155 aa, synthetic construct
  REGION 1..155: note = Synthetic
YISYDPKELCKETCLLYEIKWGMSWKIWRSSGKNTTNHVEINFIEKFTSERHFHLSVSCSITWFLSWSPCWECSQAIREFLSQHPGVTLVIYVARLFQHMDQQNRQGLRDLVNSGVTIQMMTVSEYYHCWRNFVNYPPGEEAHWPRHPPLWLMLY

SEQ ID NO: 450 - protein, 154 aa, synthetic construct
  REGION 1..154: note = Synthetic
DIFYDPRELRKEACLLYEIKWGMSPKIWRSSGKNTTNHVEVNFIEKLTSERRFHSSISCSITWFLSWSPCWECSQAIREFLSQHPGVTLVIYVARLFWHTDQQNRQGLRDLVNSGVTIQIMTASEYYHCWRNFVNYPPGEEAHWPRYPPLWMML

SEQ ID NO: 451 - protein, 154 aa, synthetic construct
  REGION 1..154: note = Synthetic
DVFYDPRELRKEACLLYEIKWGMSQKIWRSSGKNTTNHVEVNFIKKFTSEGRFQSSISCSITWFLSWSPCWECSQAIREFLSQHPGVTLVIYVARLFWHMDQQNRQGLRDLVNSGVTIQIMRASEYYHCWRNFVNYPPGDEAHWPRYPPLWMML

SEQ ID NO: 452 - protein, 155 aa, synthetic construct
  REGION 1..155: note = Synthetic
FDVFYDPRELRKEACLLYEIKWGMSRKIWRSSGKNTTNHVEVNFIKKFTSERHFHPSISCSITWFLSWSPCWECSQAIREFLSQHPGVTLVIYVARLFWHMDQQNRQGLRDLVNSGVTIQIMRASEYYHCWRNFVNYPPGDEAHWPQYPPLWMML

SEQ ID NO: 453 - protein, 98 aa, synthetic construct
  REGION 1..98: note = Synthetic
ERSLNPLTHCSVTWFLSWSPCWKCSQSVVEFRKAYPKVNLEIYVARLFRHEEECNRQGLRDLVMNGVTIRVMNLSAYNYCWRTFVSHQGDDYWPWHLT

SEQ ID NO: 454 - protein, 150 aa, synthetic construct
  REGION 1..150: note = Synthetic
FQENYMPSTWPKVTHLLYEIRWGKGSKVWRNWCSNTLTQHAEVNCLENAFGKLQFNPPVPCHITWFLSWSPCCQCCRRILQFLRAHSHITLVIKAAQLFKHMDERNRQGLRDLVQSGVHVQVMDLPDYRYCWRTFVSHPEGEGDFWPWFF

SEQ ID NO: 455 - protein, 134 aa, synthetic construct
  REGION 1..134: note = Synthetic
FQINYSPSQHRRGVYLLYEIRWRRGSIWRNWCSNTHRQHAEVNFLENCFKDRPQVPCSITWFLSASPCGKCSKRILEFLKSRPYVTLKIYAAKLFRHHDIRNREGLCNLGMHGVTIHIMNLEDYSYCWRNFVVY

SEQ ID NO: 456 - protein, 135 aa, synthetic construct
  REGION 1..135: note = Synthetic
FQRNYSPSQNGRVVYLLYEIRWKGGSIWRNWCSNNPEQHAEINFLENRFNDRPQTSCSITWFLSTSPCGKCSKRILEFLRSHPNVTLKIYTAKLFRHYEIRNRQGLRNLIMNGVAVHIMNLEDYSYCWTNFVAHQ

SEQ ID NO: 457 - protein, 134 aa, synthetic construct
  REGION 1..134: note = Synthetic
FQRNYSPRQHGRVVYLLYEIRWRRGSIWRNWCLNNHEQHAEVNFLENHFNDRPQTPCSITWFLSTSPCGKCSRRILDFLRSHPNVTLVIYAAKLFKHHDIRNRQGLRNLNMNGVTIRIMNVEDYRYCWRNFVAY

SEQ ID NO: 458 - protein, 134 aa, synthetic construct
  REGION 1..134: note = Synthetic
FKRNYLPGQHPQVVYLLYEIRWRNGSIWRNWFSNNRNQHAEVNFLENCFSDVPPAPCSITWFLSTSPCGKCSRRILEFLRTHRNVTLEIYAAKLFRHQDIRNRQGLCNLVMNGVTIHIMNLADYSYCWKRFVAY

SEQ ID NO: 459 - protein, 134 aa, synthetic construct
  REGION 1..134: note = Synthetic
FQRNYLPDQHPQAVYLLYEFRWRRGSIWRKWCSNNRAQHAEVNFLENCFNGIPPVPCSITWFLSTTPCGNCSRRILEFLRLHPNVTLEIYAAKLFRHTDIRNRKGLYNLAMNGVIIRIMNLADYSYCWRNFVAY

SEQ ID NO: 460 - protein, 119 aa, synthetic construct
  REGION 1..119: note = Synthetic
RNYLPGCYPKVVYLLYEIKWRRGTTWRNWCSNSRNLHAEVNFLENCFKAVPSVSCSMTWFLSAIPCGKCSRRILEFLKVYPNVTLEIYAAKLFKHLDIRNRQGLRNLAMNGVIIRIMNL

SEQ ID NO: 461 - protein, 134 aa, synthetic construct
  REGION 1..134: note = Synthetic
FKRNYQPGRRPNVVYLLYEIRWRRGTIWRNWCSNEFPQHAEDNFFQNRFNAVPSVSCSITWFLSTTPCGRCSKRILEFLRLHPNVTLKIYAARLFRHLDNRNRQGLRKLASNGVIIQIMGLPDYSYSWKKFVAY

SEQ ID NO: 462 - protein, 134 aa, synthetic construct
  REGION 1..134: note = Synthetic
FKTNYSPDHRPRVVYLLYEIRWRRGTIWRNWCSNNIDQHAEVNFLENCFKAKPSVSCSITWFLSTAPCAKCSRRILKFLTAHPKVTLEIYAAKLFRHLEIRNRQGLMDLAVNGVILRIMNLADYSYCWKQFVAY

SEQ ID NO: 463 - protein, 133 aa, synthetic construct
  REGION 1..133: note = Synthetic
KLNYVPVGRPRVVYLLYEIRWSRGSIWRNWCSNSSTQHAEVNFLENCFKAMPSVSCSITWFLSTTPCGNCSRRILEFLRAHPKVTLAIHAAKLFKHLDVRNRHGLKALATDGVVLHIMSIADYRYCWTKFVAY

SEQ ID NO: 464 - protein, 134 aa, synthetic construct
  REGION 1..134: note = Synthetic
FKRNYLPGQHPKVVYLLYEIRWSRGTIWRNWCSNNSTQHAEVNFLENCFKATPSVSCSITWVLSTTPCGKCSRRILEFLRVHPNVTLEIYAAKLFKHLDIRNRKGLRDLAMNGVIIRIMNLSDYSYCWKTFVAY

SEQ ID NO: 465 - protein, 134 aa, synthetic construct
  REGION 1..134: note = Synthetic
FKRNYLPGQHPEVVYLLYEIKWNSGTIWRNWCSNNPTQHAEVNFLENHFNVMSSVSCSITWGISTTPCGKCSRRILEFLTTHPNVTLEIYAAKLFKHLDIRNRQGLRNLAMNGVVICIMNLADYSYFWKTFVAY

SEQ ID NO: 466 - protein, 133 aa, synthetic construct
  REGION 1..133: note = Synthetic
IRNYLPDKHPNVVYLLYEIRWSRGTIWRNWCSNNSTQHAEVNFLENCFKAMPSVSCSITWFLSTTPCGRCSRRILKFLRVHPNVTLKIHAAKLFKHLDMRNRQGLKNLAMHGVIIRIMNLADYSYCWKTFVAH

SEQ ID NO: 467 - protein, 132 aa, synthetic construct
  REGION 1..132: note = Synthetic
RNYLPVQYPNMVYLLYEIRWSTGTIWRNWCSNNSTQHAEVNFLENRFNSRPSVSCSITWVLSTTPCGKCSTKILEFLRLHPNVTLKIYAAKLFKHLDIRNRQGLRNLAMNGVIIRIMNLADYSYCWKTFVAY

SEQ ID NO: 468 - protein, 134 aa, synthetic construct
  REGION 1..134: note = Synthetic
FKRNFLPGQHPKVVYLMYEIRWIRGTAWRSWCSNNSKQDAEVNLLENCFKAMPSVFCSVTWVLFTTPCGKCFRRILEFLRVHSNVALERYAAQLFRHLDICNWQGIRSLAMNGVIIHIMNLADYSYCWKRFVAY

SEQ ID NO: 469 - protein, 134 aa, synthetic construct
  REGION 1..134: note = Synthetic
FKRNYSPGQHPKVVYLLYEIRWSRGTTWRNWCSNNSTQHAEVNFLENCFKAMPSVFCSITWVLSTTPCGKCSRRIQEFLRVHPNVTLEIYAAKLFKHLDRRNRQGLRNLAMNGVIIRVMNLADYRYCWKRFVAY

SEQ ID NO: 470 - protein, 121 aa, synthetic construct
  REGION 1..121: note = Synthetic
FKINNLPGQHPRVVCLLYAIRWSRSTLWKSWCSNNSTQHAEVNFLENCFKGNPSVFCFMTWFFHTTPHGKCCRRTPEFLGVHPNVTLKIRAAKLFKHLDRYNQQGLRNVAMNGVVIRIINL

SEQ ID NO: 471 - protein, 134 aa, synthetic construct
  REGION 1..134: note = Synthetic
FKRNYLPAQHPKVVYLLYEIRWSRGTIWRNWCSNNSTQHAEVNFLENCFKAMPSVSCSITWFLSTTPCGKCSRRILTFLREHPNVTLEIYAAKLFKHLDVRNQQGLRNLDRNGVIIRIMNFADYSYCWKRFVAY

SEQ ID NO: 472 - protein, 134 aa, synthetic construct
  REGION 1..134: note = Synthetic
FKRNFLPSKYPKVVYLLYEIRWSSGTIWRSWCSNNSTQHAEVNFLENCFKAMPSVSCSITWVLPITPCGKCSKKILEFLSVHPNVTLEIYAAKLFRHLDIRNQQGLRNLAMNGVIIRIMNLADYSYSWKRFVAY

SEQ ID NO: 473 - protein, 134 aa, synthetic construct
  REGION 1..134: note = Synthetic
FKRNYLPGQHPKVVYLLYEIRWSRGTIWRSWCSNNSKQHAEVNFLENCFKARPSVSCSITWVLSTTPCGKCSRRILEFLRVHPNVTLEIYAAKLFKHLDIRNQQGLRNLAMNGVIIRIMNLADYSYCWKRFVAH

SEQ ID NO: 474 - protein, 134 aa, synthetic construct
  REGION 1..134: note = Synthetic
FKRNYMPSQYPKVVYLLYEIRWSRGTVWRNWCSNSFTQHAEVNFLENYFKPMPSVSCSITWVLSTTPCGKCSRRILEFLRVHPNVTLEIYAAKLFKHLDIRNRQGLRDLAMNGVTIRIMNLADYSFCWKRFVAY

SEQ ID NO: 475 - protein, 134 aa, synthetic construct
  REGION 1..134: note = Synthetic
FKRNYLPGQHPKVVYLLYEIRWSRGTIWRNWCSNNSTQHAEVNFLENCFKAMPSVSCSITWVLSTTPCGKCSRRILEFLRVHPNVTLEIYAAKLFKHLDIRNRQGLRNLAMNGVIIRIMNLADYSYGWKRFVAY

SEQ ID NO: 476 - protein, 134 aa, synthetic construct
  REGION 1..134: note = Synthetic
FKRNYSPCQHPKVVYLLYEIRWRRGAIWRSWCSNNSTQHAEVNFLENCFRAMPSASCSITWVLSTSPCGKCSRRILEFLRVHPNVTLEIYAARLFKHLDTRNRQGLRNLAMEGVVIRIMNLADYSYWWKRFVTY

SEQ ID NO: 477 - protein, 133 aa, synthetic construct
  REGION 1..133: note = Synthetic
RSNYLPCQHPRVVYLLYEIRWSRGTIWRNWCSNNSTQHAEVNFLENCFKAMPSVPCSITWVLSTTPCGKCSRRILEFLRVHPNVTLEIYAAKLFKHLDIRNRQGLRNLAKNGVVIRIMKLADYSYWWKRFVAY

SEQ ID NO: 478 - protein, 134 aa, synthetic construct
  REGION 1..134: note = Synthetic
FKRNYLPGQHPKVVYILYEIRWSRGTIWRNWCTNNSTQHAEVNFLENCFKAMPSVSCSITWVLSTTPCGKCSKRIQDFLRIYPNVTLEIHAAKLFKHLDTRNREGLRNLAKDGVIIHIMNLADYSYWWKRFVAY

SEQ ID NO: 479 - protein, 134 aa, synthetic construct
  REGION 1..134: note = Synthetic
FKRNYLPYQHPKVVCLLYEIRWNRGTIWRSWCSNNSTQHAEVNFLENCFKAKPSVSCSITWVLSTTPCGECSRRILDFLSVYPNVTLKIYAAKLFKHLDNRNRQGLWNLANNRVIIRIMNLEDYNYYWKRFVAY

SEQ ID NO: 480 - protein, 129 aa, synthetic construct
  REGION 1..129: note = Synthetic
FKRNYLPNRHPKVVCLLYEIRWSRGTIWRNWCSNSSTQHAEVNFLENCFKANPSVSCSITWVLSTTPCGKCSRRILDFLSGYPNVTLEIYAAKLFKHLDNRNRQGLWNLANNRVSIHIMNLAGSGKLLV

SEQ ID NO: 481 - protein, 133 aa, synthetic construct
  REGION 1..133: note = Synthetic
FKGNYLPDKHPRVVYLLYEIRWSRGTIWRNWCSNSSTQHAEINFLENCFKTSKTVSCSIIWVLSTTPCGKCSRRILEFLREHPNVTLEIHAAKLFKHLDTRNQQGLRDLAMKGVIIHIMNVADYSYWWKRFVA

SEQ ID NO: 482 - protein, 134 aa, synthetic construct
  REGION 1..134: note = Synthetic
FKRNYLPGKHPRVVYLLYEIRWSRGTIWRSWCSNNATQHAEINFLETCFLARTSVSCSITWVLSTTPCGKCSRRILEFLNAYPNVTLEIYAAKLFRHLDNRNRQGLRNLAMKGVRIHIMNLADYSYFWKIFVAY

SEQ ID NO: 483 - protein, 134 aa, synthetic construct
  REGION 1..134: note = Synthetic
FTRNYLPNQHPRVVYLLYEIRWRRGTIWRNWCSNNSTQHAEIKFLENCFNATTSVSCSIIWFLSTTPCGKCSTRILEFLRAHPNVTLEIYAAKLFKHHDNRNRRGLWNLAMNGVKLHIMNPADYSYCWKMFVAY

SEQ ID NO: 484 - protein, 160 aa, synthetic construct
  REGION 1..160: note = Synthetic
FHLQFNNLHRPCRRKTYLCYQLRLGSLCDQDYFQNKDLHAEIRFIKKIRSLDLDQSHNYEVTCYLTWSPCPDCAQELVALTRSHPHVRLTLFTSRLYFHWLWRFQEGLRLLWRSGVQIRVMSLREFTHCWVKFVNHGGCPFEPWDGLEQRSQSIQNRLNR

SEQ ID NO: 485 - protein, 151 aa, synthetic construct
  REGION 1..151: note = Synthetic
NRHRVRYQRKTYLCYLLEQNGQQPLKGCLQNKGKHAEILFIDEMRSLELGQVQITCYLTWSPCPNCAQELAAFKSDHPDLVLRIYTSRLYFHWRRKYQEGLCCLWRSGIQVDVMDLPQFADCWTNFVNQSPFWPWNNLEKNSRCIQRRLQR

SEQ ID NO: 486 - protein, 151 aa, synthetic construct
  REGION 1..151: note = Synthetic
NSHRVRYRRKSYLCYQLERNGQEPLKGYLLYKGQHVEILFLEKMRSMELSQVRITCYLTWSPCPNCARQLAAFKKDHPDLILRIYTSRLYFYWRKKFQKGLCTLWRSGIHVDVMDLPQFADCWTNFVNQRPFRPWNELEKNSWRIQRRLRR

SEQ ID NO: 487 - protein, 155 aa, synthetic construct
  REGION 1..155: note = Synthetic
FYSQFYNQRVHGVKPYLCYQLEQNGQAPLKGCLLSEGQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNKRPFWPWKGLEIISRRTQRRLHR

SEQ ID NO: 488 - protein, 160 aa, synthetic construct
  REGION 1..160: note = Synthetic
FRIQFNNAYKPHRRVTYLCYQVQKNGNLLTKGCLRTKGYHAESRFIKRICSLGLDQAQSYQVTCFLTWSPCPRCAQELVLFKSSHPHLYLRIFTARLYFHWRKSYQEGLQRLCRAQVPVAVMGYPEFAHCWYNFVDHPGPFEPWYKMEYYSKCIKKRFQR

SEQ ID NO: 489 - protein, 160 aa, synthetic construct
  REGION 1..160: note = Synthetic
FRVQFNNAYKPRRRVTYLCYQLQENGDPLTKGCLRTKGYHAESRFIKRICSMDLGQDQSYQVTCFLTWSPCPHCAQELVSFKRAHPHLRLQIFTARLFFHWKRSYQEGLQRLCRAQVPVAVMGHPEFAYCWDNFVDHPGPFEPWAKLEYYSSCLKRRLQQ

SEQ ID NO: 490 - protein, 160 aa, synthetic construct
  REGION 1..160: note = Synthetic
FNKQFGNQPRPYRRKTYLCYQLKGNGSILAQGCVRNKQRHAEIRFIDKINFMNLNPNQSYEIICYVTWSPCPTCAEKLVDLINDQVHLKLQIFASRLYFHWVRKYQIGLQYLWASQVTVAVMNRQEFKDCWEKFVDNGKDFQGWYKLEEYNRSISRRLNR

SEQ ID NO: 491 - protein, 160 aa, synthetic construct
  REGION 1..160: note = Synthetic
FKQQFGNQPRPYRRKTYLCYQLKQNDLTLDRGCFRNKQRHAEIRFIDKINSLDLNPSQSYKIICYITWSPCPNCANELVNFITRNNHLKLEIFASRLYFHWIKSFKMGLQDLQNAGISVAVMTHTEFEDCWEQFVDNSRPFQPWDKLEQYSASIRRRLQR

SEQ ID NO: 492 - protein, 158 aa, synthetic construct
  REGION 1..158: note = Synthetic
NIQLTNPYPKRTYLCYQLMPNGSTPTRGYFKNKNRHAEICFIDEIESMGLDKTQCYEVTCYLTWSPCPSCAQKLVAFAKAQVHLNLRIFASRLYYHWLLSCKKGLQLLWKSQIPVEVMGLPEFTDCWENFVVHGPPPFNPSEKLQELGSRSIKRRLDK

SEQ ID NO: 493 - protein, 162 aa, synthetic construct
  REGION 1..162: note = Synthetic
FSLQFNNKRRPYPRKALLCYQLTPNGSTPTRGYLKNKKNHAEIRFINKIKSMGLDETQCYQVTCYLTWSPCPSCAGKLVDFLKAHRHLNLSIFASRLYYHWRPNYQEGLLLLCGSQVPVEVMGLPEFTDCWENFVDHEPPSFNPSEKLEELDSRAIKRRLER

SEQ ID NO: 494 - protein, 162 aa, synthetic construct
  REGION 1..162: note = Synthetic
FSLQFNNKHHPYRRKALLCYQLTPNGSTPTRGQLQNKKDHAEIRFINKIKSMGLDETQCYQVTCYLTWSPCPSCARELVDFIKAHNHLNLSIFASRLYYHWRPHYQEGLLLLCGSRVPVEVMGLPEFTDCWENFVDHKPPSFNPSEKLDELDSQAIKRRLER

SEQ ID NO: 495 - protein, 162 aa, synthetic construct
  REGION 1..162: note = Synthetic
FSLQFNNKRRPYPRKALLCYQLTPNGSTPTRGHLKNKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKPPSFNPSEKLKELDSQAIKRRLER

SEQ ID NO: 496 - protein, 162 aa, synthetic construct
  REGION 1..162: note = Synthetic
FRLQFNNKRRPYPRKALLCYQLTPNGSTPTRGYFKNKKRHAEIRFINKIKSMGLDETQCYQVTCYLTWSPCPSCAQELADFIKAHDHLNLRIFASRLYCHWCRRQQEGLRLLCGSQVPVEVMGFSEFADCWENFVDYEPLSFNPSEMLEELDSRAIKRRLEK

SEQ ID NO: 497 - protein, 162 aa, synthetic construct
  REGION 1..162: note = Synthetic
FSLQFNNKRRPYPRKALLCYQLTPNGSTPTRGYFKNKKCHAEIRFINEIKSMGLDETQCYQVTCYLTWSPCPSCVRELVAFIKAHDHLNLRIFASRLYCHWCRRQQEGLRLLCGSQVPVEVMGSREFADCWENFVDHKPLSFNPSEMLEELDSRAIKRRLER

SEQ ID NO: 498 - protein, 162 aa, synthetic construct
  REGION 1..162: note = Synthetic
FRLQFNNRRRPYPRKALLCYQLTPNGSTPTRGYFENKKCHAEICFINEIKSMGLDETQCYQVTCYLTWSPCSSCAWKLVDFIQAHDHLNLRIFASRLYYHWCKPQQEGLRLLCGSQVPVEVMGLPEFNDCWENFVDHEPLSFDPCKMLEELDSRAIKRRLER

SEQ ID NO: 499 - protein, 162 aa, synthetic construct
  REGION 1..162: note = Synthetic
FRLQFNNKRRPYPRKALLCYQLTPNGSTPTRGYFENKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEPLSFNPYKMLEELDSRAIKRRLER

SEQ ID NO: 500 - protein, 161 aa, synthetic construct
  REGION 1..161: note = Synthetic
FLYHFKNVRWAGRHETYLCYVVKRDSATSFLDFGYLRNKGCHVELIFLRYISAWDLDPSRCYRVTWFTSWSPCYDCARHVANFLRCYPNLTLRIFTARLYFCEDKKPEGLRRLHRAGVQIAIMTFKDYFYCWNTFVENERTFKAWEGLHENSVRLSRQLRR

SEQ ID NO: 501 - protein, 161 aa, synthetic construct
  REGION 1..161: note = Synthetic
FLYHFKNVRWAGRHETYLCFVVKRDSATSFLDFGYLRNKGCHVELLFLQYISAWDLDPSRCYRVTWFTSWSPCYDCARHVANFLRCYPNLSLRIFTARLYFCEDKKPEGLRRLHQAGVPIAIMTFKDYFYCWNTFVENERTFKAWEGLHENSVRLSRQLRR

SEQ ID NO: 502 - protein, 161 aa, synthetic construct
  REGION 1..161: note = Synthetic
FLYHFKNVRWAGRHETYLCYVVKRDSATSFLDFGHLRNKGCHVELLFLRYISDWDLDPGRCYRVTWFTSWSPCYDCARHVADFLRGYPNLSLRIFAARLYFCEDRKPEGLRRLHRAGVQIAIMTFKDYFYCWNTFVENERTFKAWEGLHENSVRLSRQLRR

SEQ ID NO: 503 - protein, 161 aa, synthetic construct
  REGION 1..161: note = Synthetic
FLYQFKNVRWAGRHETYLCYVVKRDSATSFLDFGHLRNKGCHVELLFLRYISDWDLDPGRCYRVTWFTSWSPCYDCARHVTDFLRGYPNLTLRIFTARLYFCEGRKPEGLRRLHRAGVQIAVMTFKDYFYCWNTFVANERTFEAWEGLHENSVRLTRQLRR

SEQ ID NO: 504 - protein, 120 aa, synthetic construct
  REGION 1..120: note = Synthetic
MYPLWGRHETYLCYVVKRDSATSFLDFGHLRNKGCHVELLFLRYISDWDLDPDRCYRVTWFTSWSPCYDCARHVADFLRGYPNLTLRIFTARLYFCEDQKPEGLRRLHRAGVQLAIMTFK

SEQ ID NO: 505 - protein, 162 aa, synthetic construct
  REGION 1..162: note = Synthetic
FIYQFKNVRWAGRHETYLCYVVKRDSPTSFLDFGHIRNKGCHVELLFLRYISDWDLDPGRCYRVTWFTSWSPCYDCARHVADFLRGYPNLSLRIFTARLYFCDKERKPEGLRQLHRAGVQIAIMTFKDYFYCWNTFVENERTFKAWEGLHENSVRLSRQLRR

SEQ ID NO: 506 - protein, 161 aa, synthetic construct
  REGION 1..161: note = Synthetic
FLYQFKNVRWAGRHETYLCYVVKRDSATSCLDFGHLRNKGCHVELLFLRYISDWDLDPGRCYRVTWFTSWSPCYDCARHVADFLRGNPNLSLRIFTARLYFCEDRKPEGLRRLHRAGVQIAVMTFKDYFYCWNTFVENEKTFKAWEGLHENSVRLSRQLRR

SEQ ID NO: 507 - protein, 151 aa, synthetic construct
  REGION 1..151: note = Synthetic
FLYQFKNVRWAGRRETYLCYVVKRDSATSFLDFGYLRNKGCHVELLFLRYISDWDLDPGRCYRVTWFTSWSPCYDCARHVADFLRGNPNLSLRIFTARLYFCEDRKPEGLRRLHRAGVQIAIMTFKENERTFKAWEGLHENSVRLSRQLRR

SEQ ID NO: 508 - protein, 161 aa, synthetic construct
  REGION 1..161: note = Synthetic
FLYQFKNVRWAGRRETYLCYVVKRDSATSFLDFGYLRNKGCHVELLFLRYISDWDLDPGRCYRVTWFTSWSPCYDCARHVADFLRGNPNLSLRIFTARLYFCEDRKPEGLRRLHRAGVQIAIMTFKDYFYCWNTFVENERTFKAWEGLHENSVRLSRQLRR

SEQ ID NO: 509 - protein, 149 aa, synthetic construct
  REGION 1..149: note = Synthetic
FLYHFKNVRWAGRHETYLCYVVKRDSATSFLDFGYLRNKGCHVELLFLRYISDWDLDPGRCYRVTWFTSWSPCYDCARHVADFLRGNPNLSLRIFTARLTGWGPAGLMSPARPSDYFYCWNTFVENERTFKAWEGLHENSVRLSRRLRR

SEQ ID NO: 510 - protein, 161 aa, synthetic construct
  REGION 1..161: note = Synthetic
FLYHFKNVRWAGRHETYLCYVVKRDSATSFLDFGHLRNQGCHVELLFLRYISDWDLDPGRCYRVTWFTSWSPCYDCARHVADFLRGNPNLSLRIFTARLYFCDGFKPEGLRRLHRAGVQIAIMTFKDYFYCWNTFVENEKTFKAWEGLHENSVRLSRQLRR

SEQ ID NO: 511 - protein, 161 aa, synthetic construct
  REGION 1..161: note = Synthetic
FLYQFKNVRWAGRHETYLCYVVKRDSATSFLDFGHLRNKGCHVELLFLRYISDWDLDPGRCYRVTWFTSWSPCYDCARHVANFLRGNPNLSLRIFTARLYFCDGYKPEGLRRLHRAGVQIAIMTFKDYFYCWNTFVENERSFKAWEGLHENSVRLTRQLRR

SEQ ID NO: 512 - protein, 161 aa, synthetic construct
  REGION 1..161: note = Synthetic
FLYHFKNVRWAGRHETYLCYVVKRDSATSFLDFGHLRNKGCHVELLFLRYISDWDLDPGRCYRVTWFTSWSPCYDCARHVADFLRGNPNLSLRIFTARLYFCEDYKPEGLRRLHRAGAQIAIMTFKDYFYCWNTFVENERTFRAWEGLHENSVRLSRQLRR

SEQ ID NO: 513 - protein, 161 aa, synthetic construct
  REGION 1..161: note = Synthetic
FLYHFKNVRWAGRHETYLCYVVKRDSATSFLDFGYLRNTGCHVELLFLRYISDWDLDPGRCYRVTWFTSWSPCYDCARHVADFLRGNPNLTLRIFTARLYFCEDRKPEGLRRLHQAGVQLGIMTFKDYFYCWNTFVENERTFKAWEGLHENSVRLSRQLRR

SEQ ID NO: 514 - protein, 161 aa, synthetic construct
  REGION 1..161: note = Synthetic
FLYHFKNVRWAGRHETYLCYVVKRDSATSFLDFGHLRNKGCHVELLFLRYISDWDLDPGRCYRVTWFTSWSPCYDCARHVAEFLRWNPNLSLRIFTARLYFCEDRKPEGLRRLHRAGVQIGIMTFKDYFYCWNTFVENERTFKAWEGLHENSVRLTRQLRR

SEQ ID NO: 515 - protein, 161 aa, synthetic construct
  REGION 1..161: note = Synthetic
FLYHFKNLRWAGRHETYLCYVVKQNSATSCLDFGYLRNKGCHVEVLFLRYISTWDLDPRHCYRITWFTSWSPCYDCARHVADFLSAYPNLSLRIFAARLYFCEERNPEGLRRLHRAGAQIAIMTFKDYFYCWNTFVENKTTFKAWEGLHENSVRLARRLRR

SEQ ID NO: 516 - protein, 162 aa, synthetic construct
  REGION 1..162: note = Synthetic
FLYHYKNLRWAGRHETYLCYIVKRYSSVSCLDFGYLRNRGCHAEMLFLRYLSVWVGHDPHRAYRVTWFSSWSPCYDCAKRTLEFLKGHPNFSLRIFSARLYFCEERNPEGLRKLQKAGVRLAVMSYKDYFYCWNTFVESERRFEAWDGLHENSVRLARKLRR

SEQ ID NO: 517 - protein, 124 aa, synthetic construct
  REGION 1..124: note = Synthetic
FLYHYKNVRWAGRHETYLCYIVKRYNPASYLDFGFLRNKGCHVEMLFLRFLTGWNIDPTLPYSVTWFTSWSPCYDCSQHVTHFLRVYPNLRLRIFTARLYFCEENNPEGLRNLHMAGVQLGVMT

SEQ ID NO: 518 - protein, 160 aa, synthetic construct
  REGION 1..160: note = Synthetic
FIYHYKNVRWAGRHETYLCFVVKRDGPDTLFDFGHLRNRGCHVELVFLRHLGALCPGLSYSVTWFCSWSPCYNCSRRLAHFLTRTPNLKLRIFCSRLYFCDVEDSSEGLRLLKRAGVQLSVMTYKDYFYCWQTFVARERGFKAWEGLHQNSVRLARKLNR

SEQ ID NO: 519 - protein, 160 aa, synthetic construct
  REGION 1..160: note = Synthetic
FIFHYKNVRWAGRHETYLCFVVKRIGPDSLFDFGHLRNRGCHVELLFLRHLGALCPGLCYSVTWFCSWSPCSKCAQQLAHFLSQTPNLRLRIFVSRLYFCDEEDSREGLRHLKRAGVQISVMTYKDFFYCWQTFVARERSFKAWDGLHENSVRLVRKLNQ

SEQ ID NO: 520 - protein, 160 aa, synthetic construct
  REGION 1..160: note = Synthetic
FLYHYKNVRWAGRNETYLCFVVKRVGPDSLFDFGHLRNRGCHVELLFLRQLGTLCPGLSYSITWFCSWSPCANCSSRLAQFLKQTPNLRLRIFVSRLYFCDMEDSREGLRLLKKVGVHITVMSYKDFFYCWENFVAQQSKFKAWEGLHQNTVRLARKLNR

SEQ ID NO: 521 - protein, 160 aa, synthetic construct
  REGION 1..160: note = Synthetic
FIFHYKNVRWAGRHETYLCFVVKRVGPDTLFDFGHLRNRGCHVELLFLRYLGALCPGLSYSVTWFCSWSPCADCSFRLSQFLNRTPNLRLRIFVSRLYFCDMENSREGLRMLKNAGAHITVMSYKDFFYCWQTFVARESNFKAWDELHRNSVRLSRKLHR

SEQ ID NO: 522 - protein, 160 aa, synthetic construct
  REGION 1..160: note = Synthetic
FIYHYKNVRWAGRHETYLCFVVKRVGPDTLFDFGHLRNRGCHVELLFLRYLGALCPGLSYSVTWFCSWSPCVNCSIQLCQFLNNTPNLRLRIFVSRLYFCDLEDSREGLRMLTKAGVRISVMSYKDYFYCWQKFVDCKSNFKAWEELHQNSVRLTRKLNR

SEQ ID NO: 523 - protein, 160 aa, synthetic construct
  REGION 1..160: note = Synthetic
FIYHYTNMRWAGRHETYLCFVVKRVGPDSLFDFGHLRNRGCHVELLFLRHLGALCPGFSYSITWFCSWSPCVNCSISLSQFLSRTPNLRLRIFVSRLYFCDMENSRDGLRMLKKAGVQVTVMSYKDFFYCWQTFVDRQSQFKAWKELHQNSVRLSRKLKR

SEQ ID NO: 524 - protein, 160 aa, synthetic construct
  REGION 1..160: note = Synthetic
FIYHYKNVRWAGRHETYLCFVVKRIGPNSLFDFGHLRNRGCHVELSEIQSFSAFCPALVQNDSKSCRISTDSESLHQIETSLDKTPKTGIKVFLSLSLFVTLANHSHYPQMASDLCLCVPIFLFTDFFYCWQTFVARESRFKAWDGLHQNSVRLSRKLKR

SEQ ID NO: 525 - protein, 160 aa, synthetic construct
  REGION 1..160: note = Synthetic
FIYHYKNMRWAGRHETYLCFVVKRVGPESLFDFGHLRNRGCHVELLFLRHLSALCPGLSYSITWFCSWSPCANCSFRLAQFLSQTPNLRLRIFVSRLYFCDLEDSREGLRMLKKVGVHITVMSYKDYFYCWQTFVARQSKFKPWDGLHQNSVRLSRKLNR

SEQ ID NO: 526 - protein, 160 aa, synthetic construct
  REGION 1..160: note = Synthetic
FIYHYKNLRWAGRCETYLCFVVKKVGPDSLFDFGHLRNRNCHVELLFLRHLGALCPGLSYSVTWFCSWSPCANCSIRLAQFLHQTPNLRLRIFVSRLYFCDLEDSREGLRILKKAGVHITVMSYKDYFYCWQTFVAKQSKFKPWDGLHQNYIRLSRKLNR

SEQ ID NO: 527 - protein, 161 aa, synthetic construct
  REGION 1..161: note = Synthetic
FIYHYKNLRWAGRCETYLCFVVKKVGRNRLFDLNVTMNNKPLHLQLLFLRHLGALCPGLSYSVTWFCSWSPCANCSIRLAQFLHQTPNLRLRIFVSRLYFCDLEDSREGLRILKKAGVHITVMSYKDYFYCWQTFVAKQSKFKPWDGLHQNYIRLSRKLNR

SEQ ID NO: 528 - protein, 161 aa, synthetic construct
  REGION 1..161: note = Synthetic
FLYNFKNLRWAGRHETYLCYVVKRDSATSFLDFGYLRNKGCHVEMLFLRYISAWDLDPGRCYRVTWFTSWSPCYDCARHVADFLRAYPNLTLRIFAARLYFCEDRNPEGLRRLHRAGVQIAIMTFKDYFYCWNTFVENERTFKAWEGLHENSVRLSRRLRR

SEQ ID NO: 529 - protein, 161 aa, synthetic construct
  REGION 1..161: note = Synthetic
FLYNFKNLRWAGRHETYMCYVVKRDSATSCLDFGYLRNKGCHVEVLFLRYISAWDLDPGRCYRVTWFTSWSPCYDCARHVADFLRAYPNLTLRIFVARLYFCEGRNPEGLRRLHRAGAHIAIMTFKDYFYCWNTFVENERTFKAWEGLHENSVHLTRKLRR

SEQ ID NO: 530 - protein, 161 aa, synthetic construct
  REGION 1..161: note = Synthetic
FLYNFKNLRWAGRRETYLCYVVKRDSATSCMDFGYLRNKGCHVELLFLRYISAWDLDPGRCYRITWFTSWSPCYDCARHVADFLRAYPNLTLRIFAARLYFCEDRKPEGLRRLHRAGAQIAIMTFKDYFYCWNTFVENEKTFEAWEGLHENSVHLTRKLRR

SEQ ID NO: 531 - protein, 161 aa, synthetic construct
  REGION 1..161: note = Synthetic
FLYNFKNLRWAGRRETYLCYVVKRDSATSCLDFGYLRNKGCHVEVLFLRYISAWDLDPGRCYRITWFTSWSPCYDCARHVADFLRTYPNLTLRIFAARLYFCEDRKPEGLRRLHKAGAQIAIMTFKDYFYCWNTFVENEKTFKAWEGLHENSVHLSRKLRR

SEQ ID NO: 532 - protein, 161 aa, synthetic construct
  REGION 1..161: note = Synthetic
FLYNFKNMRWAGRRETYLCYVVKRNSATSCLDFGYLRNQGCHVEVLFLRYISAWDLDPGRCYRITWFTSWSPCYDCARHVADFLRAYPNLSLRIFTARLYFCEDRKPEGLRRLHRAGAQIAIMTFKDYFYCWNTFVENEKTFKAWEGLHENSVRLSRQLRR

SEQ ID NO: 533 - protein, 161 aa, synthetic construct
  REGION 1..161: note = Synthetic
FLYNFKNLRWAGRRETYLCYVVKRDSATSCLDFGYLRNQGCHVEVLFLRYISAWDLDPGRCYRITWFTSWSPCYDCARHVANFLRAYPNLTLRIFTARLYFCEDRKPEGLRRLHRAGAQIAIMTFKDYFYCWNTFVENEKTFKAWEGLHENSVHLSRKLRR

SEQ ID NO: 534 - protein, 161 aa, synthetic construct
  REGION 1..161: note = Synthetic
FLYNFKNLRWAGRRETYLCYVVKRDSATSYLDFGYLRNQGCHVEVLFLRYISAWDLDPGRCYRITWFTSWSPCYDCARHVANFLHSYPNLTLRIFTARLYFCEDRKPEGLRRLHKAGAQIAIMTFKDYFYCWNTFVENEQTFKGWEGLRENSVHLSRKLRR

SEQ ID NO: 535 - protein, 161 aa, synthetic construct
  REGION 1..161: note = Synthetic
FLYNFKNLRWAGRRETYLCYVVKRDSATSCLDFGYLRNQGCHVEVLFLRYIAAWDLDPGRCYRITWFTSWSPCYDCAQHVANFLRSYPNLTLRIFTARLYFCEDRKPEGLRRLHKAGAQIAIMTFKDFFYCWNTFVENEQTFKGWEGLHENSVHLSRKLRR

SEQ ID NO: 536 - protein, 161 aa, synthetic construct
  REGION 1..161: note = Synthetic
FLYNFRNLRKAGRRETYLCYVVKRDSATSCLDFGYLRNQGCHVEVLFLRYIAAWDLDPGRCYRITWFTSWSPCYDCAQHIANFLRSYPNLTLRIFMARLYFCEDRKPEGLRRLHKAGAQIAIMTFKDYFYCWNTFVENEQTFKGWEGLHENSVHLARKLRR

SEQ ID NO: 537 - protein, 157 aa, synthetic construct
  REGION 1..157: note = Synthetic
FTHNFSNHKRTHKTYLCYEVEIHGDSGIPDKGFLCNKCHVELYLLGRIRSWKLDRKLHCRLTCFISWTPCGTCARELAEFLKENSHVSLCIFASRIYSLNDYEAGLRTLQEAGAQIAIMTFKEFRHCWETFVDHGRPFQPWDELDINSQGLSKELQA

SEQ ID NO: 538 - protein, 157 aa, synthetic construct
  REGION 1..157: note = Synthetic
FTQNFRNDNPSKTYLCYQVELDGSSGVLDKGSAFPGGQHAEWFLLEHIRSRNLDQKLSYKVTCFLSWTPCEKCAEEIIRFLAKNRHVSLSILASRIYTMGPYVKGLRELYDAGVHISIMTFRDFEYCWQTFVDHDSPFQPWADLDRRSQQLSQQLRA

SEQ ID NO: 539 - protein, 161 aa, synthetic construct
  REGION 1..161: note = Synthetic
FTSNFTNNPAIGRRQTYLCHEVQLDGDSWVLDRGFLQSQPLHAEFCFLDRVGSWQLNPNWHYRVTCFISWSPCFSCAQKVAMFLRRNSHVKLRILAARIYDYHPGYEEGLKALQGTGAQVAIMTHAEFEHCWDTFVDHGRPFQPWEGLDKNSQALSRRLQD

SEQ ID NO: 540 - protein, 161 aa, synthetic construct
  REGION 1..161: note = Synthetic
FRFNFINDASVGQKQTYLCYEVELDGNSWVLDRGFLLNQRRHAELCFLDRVSSWHLDPTKHYKFTWFLSWSPCRNCAQEVVAFLGGNSHVSLSIFAPRIYDYYSGYEEGLRSLQGAGAHVSIMTSTEFEHCWRTFVDNGCPFVPWNRLGENSQTISRRLQS

SEQ ID NO: 541 - protein, 153 aa, synthetic construct
  REGION 1..153: note = Synthetic
FQLNFINDLSVGQKQAYLCYEVELDCNSRVLNRGFLCNQGCHAELCFLDQVPSWQLDLVLCYRVTWFISWSPGPDCAQEVAAFLRGNSHVSLSIFASCIYDSVEESEGGWKGKKDLGASKKKFGHCWKTIVDNGRFFEPWNRLDESFDFLDLT

SEQ ID NO: 542 - protein, 150 aa, synthetic construct
  REGION 1..150: note = Synthetic
FMDHFANEDGGGLNETYLCYEVQLDGSSQGFLRNKRRHAELCFLDLVPAWRLDPAQHYRVTWFISWSPCFLCAQAVAEFLRRNAHVSLRIFAARIYTWRTDYKAGLQDLQRAGAQIAIMTPAEIQFCWNTFVDNSNPFHSHLGLGHAKPA

SEQ ID NO: 543 - protein, 113 aa, synthetic construct
  REGION 1..113: note = Synthetic
CFDGNNRRPRAELCFLALFQSWHLNEGKQYRLTWYSSWSPYPDCVPKLVEFLGDNSNVSLRIFAAGIHSIFTGYKRELRNLRDAGAQLAIMTLEELRWELGGEMATWRWQNEN

SEQ ID NO: 544 - protein, 158 aa, synthetic construct
  REGION 1..158: note = Synthetic
FKENFANTWENETELCYEVEVEGDTWAVEQGFLCNQPCHAELCFLCLVRSWHVDEGKQYRLTWHISWSPCPNCAQKLVKFLHDNSHVSLRIFAAGIQTTFSGHEDWLRKLRDSGAQLAIMTLKELQHCWDTFVDNGQPFEPWPNLVEHIQTESQKLKD

SEQ ID NO: 545 - protein, 156 aa, synthetic construct
  REGION 1..156: note = Synthetic
EWNFGITWAKETYLCYEVEVEGDAWVAKEGFLRNELRHAELCFLRGVSDWDLDEGKQYRLTYMSWSPCPNCAPKLVEFLDENSHVTLRIFPARIHTKSRGYQDGLRNLRDAGAQLAIMTLKEHQHCWDTFVDNGQPFRPWPNLVEHIETKSQELKD

SEQ ID NO: 546 - protein, 157 aa, synthetic construct
  REGION 1..157: note = Synthetic
FKDNFGHNWEKTYLWYEVEFEGDAWAVEQGFLRNQLRHAELCFLHGVRSWHLDEGKQYRLTWHISWSPCPDCASKLVEFLGENSHVSLRIFAARIHTKYRGYEDGLRQLQDAVDHLTIMTLKELQHCWVTFVDNGQPFEPEIELLENIGAQCQKLES

SEQ ID NO: 547 - protein, 158 aa, synthetic construct
  REGION 1..158: note = Synthetic
FKDNFGITWANETYLCYEVEVEGDAWAVEQGFLRNQRSHAELCFLDRVPSWHLDEGKQYRLTCYISWSPCPDCAQKLVEFMGENSHVSLRIFAARIYTKLDGHEDGLRKLQDAGAQLAIMTLKEYEHCWDTFVDSGQLFRARDELEVHIGAQCQRLEN

SEQ ID NO: 548 - protein, 153 aa, synthetic construct
  REGION 1..153: note = Synthetic
FKRNFRNKCKNQTYLCYEVEVEGNAWAVEQGFLRNQRRHAELCFLDRVPCWNLDGLKKYRLTCYISWSPCPDCALELVQFLGLKSNVSLRMCTAGIRTTFPGHEDGLRNLRDAGAQLTIMTRDEYEHCWDTFVDNGQPFRARDELEGHDYFLL

SEQ ID NO: 549 - protein, 157 aa, synthetic construct
  REGION 1..157: note = Synthetic
EVNFGHDWEKKTYLCYEVEVEGDAWVGKQGFLCNQPGHAELCFLDRVRSWHLDGGKQYRLTCYMSWTPSPDCALELVQFLSENSHVSLRIFAAGIRTRFHGHEDGLRQLRDAGAQLAIMTLHELQHCWDTFVDNGQPFRARDELEVHIGAQCQKLKS

SEQ ID NO: 550 - protein, 158 aa, synthetic construct
  REGION 1..158: note = Synthetic
KRRNFRNKCENQTYLCYEVEVKDGKWTVEQGFLRNQPGHAELCFLDRVRSWHLDEGRQYRLTCYISWTPGPDCAQKLVEFLGENRHVSLRIFAAGIHTKYRGHEDGLRQLWDAGAQIAIMTLNELQHCWETFVDNGQPFEPWPIQVEHIQTESQKLKD

SEQ ID NO: 551 - protein, 131 aa, synthetic construct
  REGION 1..131: note = Synthetic
AEKLFFKKLLQQVYYCFGLKIEGRDQPRHAELCFQDRVRSWHLDEGKQYRLTCYISWSPCPDCAQKLVEFLGENSHVRLRIFAARIYKKRDRYKHWLRQLRDAGAQLTIMTLNELQHCWVTFVDNGQRFEP

SEQ ID NO: 552 - protein, 120 aa, synthetic construct
  REGION 1..120: note = Synthetic
FRVNFSYYRERKTYLCYEVEVEGDAWVVKQDFLRNQPRHAELCFLDGVRSWHLDEGKQYRLTCYISWSPCPVCAQELVEFLGENRHLRLRIFAARIYSIVSGYEDGLRQLWDAGAPLAIM

SEQ ID NO: 553 - protein, 158 aa, synthetic construct
  REGION 1..158: note = Synthetic
FKDNFSHRRARRTYLCYQVEVEGDAWAVEYGFLCNQLRHAELCFLDRVPFWNLEEGRQYRLTCYISWSPCPDCAQRLVEFLGNNNHMRLRIFAARIYTFVSGHEDGLRQLWDAGAQLTIMTRNDLQHCWDTFVDNGDPFEPCPIQVEHIGTESQELEN

SEQ ID NO: 554 - protein, 158 aa, synthetic construct
  REGION 1..158: note = Synthetic
FKENFSHRRARKTYLCYEVEVEGNTWAVEQGFLHNQLRHAELCFLDRVRFWNLEEGRQYRLTCYISWSPCPDCAQKLVEFLGQNSHVSLCIFAARIYTIVSGYKDGLCQLRDAGAQLTIMTLNDLQHCWENLVDNGEPFEPCPTLVEHIETKSQELKD

SEQ ID NO: 555 - protein, 163 aa, synthetic construct
  REGION 1..163: note = Synthetic
FTFNFNNDLSVGRHQTYLCYEVERDNGTWVMDRGFLHNKGCHAELCFLGEVPSWQLDPAQTYRVTWFISWSPCLRRGCAEQVRAFLQENTHVRLHIFAARIYDFDFLYQEALRTLRDAGAQVSIMTYEEFKHCWDTFVDHGRPFQRWDGLDEHSQDLSGRLRA

SEQ ID NO: 556 - protein, 163 aa, synthetic construct
  REGION 1..163: note = Synthetic
FTINFNNDLSVGRRQTYLCYEVERDNGTWVMDWGFLCNQSCHVELCFLSQVSSWQLDPAQTYRVTWFISWSPCFSGGCAEQVRAFLQENTHVRLCIFAARIYNYDPLYQEALRMLRDAGAQVSIMTYEEFEYCWDTFVDRGCPFQPWDGLDEHSQALSGRLRA

SEQ ID NO: 557 - protein, 163 aa, synthetic construct
  REGION 1..163: note = Synthetic
FTSNFNNDVSVGRHQTYLCYEVERDNGTWVMDWGFLCNQGCHAELCFLGWVPSWQLDPAQTYRVTWFISWSPCFSWGCAEQVRAFLQENTHVRLHIFAARIYDYDPLYQEALRTLRDAGAQVSIMTYDEFEYCWDTFVDCGCPFQPWDGLDEHSQALSERLRA

SEQ ID NO: 558 - protein, 163 aa, synthetic construct
  REGION 1..163: note = Synthetic
FTFNFNNDPLVGRHQTYLCYEVERDNGTWVMDRGFLHNQGRHTELCFLGLIPYWQLDLAQTYRVTWFISWSPCFSWGCAEQVRAFLQENTHMRLRIFAARIYDYDPLYKEALQMLRGAGAQVSIMTYDEFEHCWDTFVDHGRPFQPWDGLEEHSQALSGRLQA

SEQ ID NO: 559 - protein, 163 aa, synthetic construct
  REGION 1..163: note = Synthetic
FTFNFNNDPFVRRHQTYLCYEVEHDNGTWVMDRGSLHNQGRHAELRFLGLLPYWQLDPAQIYRVTWFISWSPCFSWGCARQVRAFLQENTHVRLRIFAARIYDYDPLYKEALQMLRDAGAQVSIMTYDEFEYCWNTFVDHGCPFQPWDGLEEHSQALSGKLQA

SEQ ID NO: 560 - protein, 160 aa, synthetic construct
  REGION 1..160: note = Synthetic
FTSNFNNGRWHKTYLCYEVERDNGTWVMDRGFLHNQGRHAELCFLDLVPSLQLDPAQTYRVTWFISWSPCFSWGCAEQVRAFLQENTHVRLRLFAARIYDYDPLYKEALQMLRGAGAQVSIMTYHEFKHCWDTFVDHGRPFQPWDGLEEHSQALSGRLQA

SEQ ID NO: 561 - protein, 160 aa, synthetic construct
  REGION 1..160: note = Synthetic
FTSNFNNGGRRKTYLCYEVERDNGTSVMDRGFLHNQGRHAELRFLDLVPSLQLDPAQIYRVTWFISWSPCFSWGCAGQVRAFLQENTHVRLRIFAARIYDYDPLYKEALQMLRDAGAQVSIMTYDEFKHCWDTFVDHGCPFQPWDGLEEHSQALSGRLRA

SEQ ID NO: 562 - protein, 163 aa, synthetic construct
  REGION 1..163: note = Synthetic
FTSNFNNDLLVRRHQTYLCYEVERDNGTWVMDRGFLHNQGRHAELRFLDLVPSLQLDPAQIYRVTWFISWSPCFSWGCAGQVCEFLQENTHMRLRIFAARIYDYDPLYKKALQMLRDAGAQVSIMTYDEFKHCWDTFVYRGCPFQPWDGLEEHSQALSGRLQA

SEQ ID NO: 563 - protein, 163 aa, synthetic construct
  REGION 1..163: note = Synthetic
FTFNFNNDPLVRRHQTYLCYEVERDNGTWVMDMGFLCNEGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRGCPFQPWDGLEEHSQALSGRLRA

SEQ ID NO: 564 - protein, 163 aa, synthetic construct
  REGION 1..163: note = Synthetic
FTFNFNNDPLVRRHQTYLCYEVERDNGTWVMDMGFLCNEGRHAELRFLDLVPSLQLDPAQIYRVTWFISWSPCFSWGCAGQVCEFLQENTHMRLRIFAARIYDYDPLYKKALQMLRDAGAQVSIMTYDEFKHCWDTFVYRGCPFQPWDGLEEHSQALSGRLQA

SEQ ID NO: 565 - protein, 157 aa, synthetic construct
  REGION 1..157: note = Synthetic
FTYNFTNDPSVGQHQTYLCYKVECDNDTWVLDKGILPNQHAEQYFLYLISFWKLDQAQCYRVTWFISWSTCFSCAQQVATFHWENRCVSLHIFIARIYNYLPGYEGLCMLQRAGTQISIMTSKFRHCWVTFVDHGHPFQPWDGLDEHSQALSGRLQA

SEQ ID NO: 566 - protein, 157 aa, synthetic construct
  REGION 1..157: note = Synthetic
FTYNFTNDPSVGQHQTYLCYKVECDNDTWVLDKGILPNQHAEQYFLYLISFWKLDRAQCYRVTWFISWSTCFSCAQQVTTFHWENRCVSLHIFVACIYNYLPGYEGLCMLQRAGTQISIMTSGFRHCWVTFVDHGHPFQPWDGLDEHSQALSGRLQA

SEQ ID NO: 567 - protein, 126 aa, synthetic construct
  REGION 1..126: note = Synthetic
FTYNFTNDPSVGRHQTYLCYEVEHHNGTWVLHRGFILNEGRHAELCLLDLILFWKLDLAQRYRVTCFISWSPCFCCAEKVAEFLQENPHVNLRIFAARIYGYQRGYKKGLRRLNRAGAPISMMKYS

SEQ ID NO: 568 - protein, 161 aa, synthetic construct
  REGION 1..161: note = Synthetic
FTYNFTNDPSVGQHQTYLCYEVEHHNGTWVLHRGFILNQGRHAELCLLDLISFWKLDLAQYYTVTCFISWSPCFSCAEKVAEFLQENPHVNLHIFAAHIYGYQRGYIKGLCRLNRAGAPISMMKYSEFSYCWDTFVDHEHPFQPWEGLDEYTQALSGKLQA

SEQ ID NO: 569 - protein, 439 aa, synthetic construct
  REGION 1..439: note = Synthetic
  VARIANT 139..142: note = X can be any naturally occurring amino acid
PHETHRSERASNPHEASNASNLYSPROTRPVALGLYGLNARGGLUTHRTYRLEUCYSTYRLYSVALGLUARGHISASNASPTHRTRPVALLEUASNARGGLYPHELEUARGASNGLNGLYARGHISALAGLULEUCYSPHELEUASPLEUILEPROPHETRPLYSLEUASPASPGLNGLNTYRARGVALTHRCYSPHETHRSERTRPSERPROCYSPHESERCYSALAGLNLYSMETALALYSPHEILESERLYSASNLYSHISVALSERLEUCYSILEPHEALAALAARGILETYRASPASPGLNGLYARGCYSGLNGLUGLYLEUARGTHRLEUHISARGASPGLYALALYSILEALAVALMETASNTYRSERGLUPHEGLUTYRCYSTRPASPTHRPHEVALASPARGGLYXXXXLEUGLYVALLYSPROASPCYS

SEQ ID NO: 570 - protein, 160 aa, synthetic construct
  REGION 1..160: note = Synthetic
FTSNFYNKPWVGQHETYLCYKVERHNGTWVLNRGFLRNQGRHAELCFLDLIPFWKLDGQQYRVTCFTSWSPCFSCAQEMAKFISNNEHVSLCIFAARIYDDQGRCQEGLRTLHRDGAKIAMMNYSEFEYCWDTFVDRGRPFQPWDGLDEHSQDLSGRLRA

SEQ ID NO: 571 - protein, 160 aa, synthetic construct
  REGION 1..160: note = Synthetic
FTSNFNNKPWVGQHETYLCYKVERHNDTWVLNRGFLRNQGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRGRPFQPWDGLDEHSQALSGRLRA

SEQ ID NO: 572 - protein, 161 aa, synthetic construct
  REGION 1..161: note = Synthetic
FTSNFNNEPCVGRHETYLCYKVERHNDTWVLNRGFLCNQGRHAELCFLDVIPFWKLDGKQRYRVTCFTSWSPCFRCAQEMAKFISNNQHVSLCIFAARIYDDQGRCKEGLRTLDEAEAKISIMTYSEFQHCWDTFVDHGRPFQPWDGLEEHSEAWSGKLQA

SEQ ID NO: 573 - protein, 161 aa, synthetic construct
  REGION 1..161: note = Synthetic
FTSNFNNEHWVGRHETYLCYEVERHNDTWVLNRGFLCNQGRHAELCFLDVIPFWKLDLHQDYRVTCFTSWSPCFSCAQEMAKFISNKKHVSLCIFAARIYDDQGRCQEGLRTLAEAGAKISIMTYSEFKHCWDTFVYHGCPFQPWDGLEEHSQALSGRLQA

SEQ ID NO: 574 - protein, 159 aa, synthetic construct
  REGION 1..159: note = Synthetic
FTSNFNNEHWVGRHETYLCYEVERHNDTWVLNRGFLCNQGRHAELCFLDVIPFWKLDLHQDYRVTCFTSWSPCFSCAQEMAKFISNKKHVSLCIFAARIYDDQGRCQEGLRTLAEAGAKISIMTYSEFKHCWDTFVDHGCPFQPWDGLEEHSQSLGDRA

SEQ ID NO: 575 - protein, 164 aa, synthetic construct
  REGION 1..164: note = Synthetic
FNFHYKNLQRAGRRKTYLCYLLEEEREGAIVLDVGVLHNQEHAERRFLSSPDILQLLDGGRRYRVTWYLSWSPCSQCARAVAGFLAQHGNVSLRIFVARLYNHEDPENRQGLRTLNSTGTPIRVMTNREFALCWERFVRHQGAAFEPWAGLRENADLLLGQLED

SEQ ID NO: 576 - protein, 159 aa, synthetic construct
  REGION 1..159: note = Synthetic
FMFQFKNVEYSGRNKTILCYTVERPEGQVFHGYLEDESAHAEDAFFTSVLPQLTSGSVTVTCYVSSSPCVNCAASVAQCLRRNKTVRIQLAVARLFQWEEPEIRRALKGLRSAGCQVRMMRGADYVYVWKNFVEPDHQDFIPWEDLEENARYYEEKLEE

SEQ ID NO: 577 - protein, 160 aa, synthetic construct
  REGION 1..160: note = Synthetic
FKFQFKNVEYSGRNKTFLCFLVDVQGSGGEGLRGYLEDEGAHAEEAFFQQVLPQDSALHYMVTWYVSSSPCAACTAKLVEILKARKTMRLTIFSARLFMWEEPEIQVGLKALAAAGCKLRMMKPTDFVYIWDTFVENDQTFTPWEDCQENYEYYQEKLAD

SEQ ID NO: 578 - protein, 160 aa, synthetic construct
  REGION 1..160: note = Synthetic
FKFQFKNVEYSGRNKTFLCYQVDIQGGETDGVRGYLEDEGSHAEEAFFQQVLAYDKSLRYTVTWYTSSSPCVACAAKLVEILKARKALRLNIHCSRLFEWEEPEIQAGLQALVRAGCKLRMMRPVDFVYVWSTFVENEDNFTPWEDCQDNFEYYDERLQD

SEQ ID NO: 579 - protein, 159 aa, synthetic construct
  REGION 1..159: note = Synthetic
FKFQFRNVEYSGRNKTLLCFRVDTAGGSTEPLRGYMEDETAHAEEAFFQQVLPNSSQEYDVTWYVSSSPCVACAAKLTSILQQRKKLRLSVFCSRLFDWEEPEIVQGLKALVQAGCKLQMMKPADFQHVWETYVEKDQSFTLWEDCKENYEYYLEKLAD

SEQ ID NO: 580 - protein, 159 aa, synthetic construct
  REGION 1..159: note = Synthetic
FKFQFRNVEYSGRNKTLLCFRVDTAGGNAEPLKGYMEDETAHAEEAFFQQVLPNSSTEYDVTWYVSSSPCVACAAKLANILQQRKKLRLSIFCSRLFEWEEPEIVEGLKALARAGCKLRMMKPIDFQHVWEMYVEQGESFTPWEDCQENYEYYVERLTD

SEQ ID NO: 581 - protein, 159 aa, synthetic construct
  REGION 1..159: note = Synthetic
FKFQFRNVEYSGRNKTLLCFRVDTPGGSTEPLKGYMEDETAHAEEAFFQQVLPNTSQEYEVTWYLSSSPCVACAAKLAHILQQRKKVRLRMFCSRLFEWEEPEIVEGLRALVSAGCKLRMMKPSDFVHVWETYVEKDQNFEAWEDCQENYDYYVEKLTD

SEQ ID NO: 582 - protein, 159 aa, synthetic construct
  REGION 1..159: note = Synthetic
FKFQFRNVEYSGRNKTLLCFRVDSPGGSTEPLKGYMEDETAHAEEAFFQQVLPNPSLEYDITWYVSSSPCISCANTLAKMLQQRKKVRLCVFCSRLFEWDQPEVVEAIRTLVRAGCKLRMMKPSDFQHVWETYVEKGESFTPWEDCQDNYNYYKEILAD

SEQ ID NO: 583 - protein, 160 aa, synthetic construct
  REGION 1..160: note = Synthetic
FKFQFKNVEYSGRNKTLLCFLVDSQKGEDELQRGYLEDEGAHAEEAFFQQVLPEDPGRRYQVTWYVSSSPCAACAAKLAEILRARPTLRLTIFSARLFMWEEPEIQEGLKALAAAGCKLRVMKPVDFSYTWDTFVENDQTFSPWEDCQENYEYYQEKLAG

SEQ ID NO: 584 - protein, 160 aa, synthetic construct
  REGION 1..160: note = Synthetic
FRFQFKNVEYCGRNKTLLCYLVDQSSGADGLLRGFLEDEGLHAELAFFQLILPQDPSVNYTVTWYVSSSPCAHCCSKLLEILQERKTLRLNIFSARLLEAEEEEGQAGMKALAGAGCKLRVMKPLDFSYSWDTFVEHDERFTPWEDCQENYEYHHEKLAQ

SEQ ID NO: 585 - protein, 160 aa, synthetic construct
  REGION 1..160: note = Synthetic
FKFQFKNVEYSGRNKTLLCYLVDTGGAGDGLLRGYLEDEGIHAEEAFFTHCLPNDPNVQYTITWYVSSSPCHACSLKLAEVLKARKNVKLSIFSARLFEWEAEEVQAGLKALHSAGCKLRVMKPLDFSYTWDTFVETEQPLNLWEDCKDNYEYYHEKLDQ

SEQ ID NO: 586 - protein, 160 aa, synthetic construct
  REGION 1..160: note = Synthetic
FQFQFKNVEYSGRNKTFLCYLVDTGKADEGLQRGYLEDEGSHAEEAFFTQCLPHDPALKYSVTWYMSSSPCCACAAKMAEALKARRNIKLSIFAARLFEWEEAEIQAGLKALHIAGCKIRVMKPLDFSYTWDTFVENDQPLNLWADCKENYEYYHERLAD

SEQ ID NO: 587 - protein, 160 aa, synthetic construct
  REGION 1..160: note = Synthetic
FKFQFKNVEYSGRNKTLLCYLVDKGNTSDGLLRGYLEDEGTHAEEAFFTQCLPHNPALKYTVTWYVSSSPCSACAAKIAELLKDQKNLKLNIFAARLFEWEEGEIQAGLRTLHAAGCKLRVMKPLDFSYTWDTFVENDQSLNLWEDCKENYEYYHEKLSD

SEQ ID NO: 588 - protein, 160 aa, synthetic construct
  REGION 1..160: note = Synthetic
FKFQFKNVEYSGRNKTFLCYLVDKGNTADGLLRGYLEDEGSHAEEVFFIQCLPHDPTLKYTVTWYVSSSPCSACAAKIAEVLKARKNVKLSIFAARLFECEEVEIQAGLKALHAAGCKLRVMKPLDFSYTWDTFVENEQPLNLWEDCKENYEYYHEKLAD

SEQ ID NO: 589 - protein, 160 aa, synthetic construct
  REGION 1..160: note = Synthetic
FKFQFKNVEYSGRNKTFLCYLVDKGNTDDGLLRGYLEDEGSHAEEAFFIQCIPNDPSVRYTVTWYVSSSPCAACAAKIAEVLKARKNIKLSIFAARLFECEETDIQAGLKAMHTAGCKLRVMKPLDFSYTWDTFVENEEPLNLWEDCKENYEYYQEKLAD

SEQ ID NO: 590 - protein, 161 aa, synthetic construct
  REGION 1..161: note = Synthetic
FKFQFKNVEYSGRNKTFLCYIVETQGKESGTIRGYLEDEAAHAEDAFFNTILPEESSLRYNVTWYVSSSPCVACAERIAEILKKNKNLRLAILVSRLFMWEEPEMQAVLKKMKAAGCKLRIMKPQDFEYVWQNFVEQESKDFVPWEDIQENFLYYEEKLAE

SEQ ID NO: 591 - protein, 161 aa, synthetic construct
  REGION 1..161: note = Synthetic
FKFQFRNVEYSGRNKTFLCYIVETQGKESGSFRGYLEDEAAHAEGAFFNNILPTEPSLRYNVTWYVSSSPCVPCADQIAQILQKNKNLRLTILVSRLFMWEEPEMQAALKKLKTAGCKLKIMKPQDFEYIWQNFVEQEAKAFEPWEDIQENFLYYEEKLAE

SEQ ID NO: 592 - protein, 161 aa, synthetic construct
  REGION 1..161: note = Synthetic
FKFQFRNVEYSGRNKTFLCYVVEIQGKEPGSFQGYLEDEAAHAEDAFFNNILPTDPNLRYNVTWYVSSSPCVPCADRIAQVLQKNKNLRLSILVSRLFMWEEPEMQAALKKLKASGCKLKIMKPQDFEYIWQNFAEQEAKAFEPWEDIQENFLYYEEKLSE

SEQ ID NO: 593 - protein, 129 aa, synthetic construct
  REGION 1..129: note = Synthetic
FNFQFKNVEYSGRNKTFLCYIIEIQGKESKMLRGYLEDEAAHAEDAFFNTILPKESGMCYNVTWYSSCSPCIGCADRIAKALQKNKNLHLSIAVGRLFMWEEPDMQAALKKMKTAGCKLRIMKPQDFEY

SEQ ID NO: 594 - protein, 161 aa, synthetic construct
  REGION 1..161: note = Synthetic
FKFQFKNVEYSGRNKTFLCYIIEMQGKESKTLRGYLEDEAAHAEEAFFNTILPTEPGFRYEVTWYVSSSPCVSCAERIVKALKKNKNLRLSIAVGRLFMWEEPDIQAALKQMKAAGCKLRIMKPQDFEYVWKNFVEQEPKAFAPWDDIQENFQYYDEKLAE

SEQ ID NO: 595 - protein, 161 aa, synthetic construct
  REGION 1..161: note = Synthetic
FKFQFRNVEYSGRNKTFLCYVVETQGRESVTSRGYLEDEAAHAEMAFFNTILPKESSARYNVTWYVSSSPCVTCADRITETLKKNKNLRLTIMVGRLFMWEEPEMQAALKNMRAAGCKLRIMKPQDFEYVWQNFVEQEAKAFAPWEDIQENFQYYEEKLAE

SEQ ID NO: 596 - protein, 161 aa, synthetic construct
  REGION 1..161: note = Synthetic
FKFQFRNVEYSGRNKTFLCYVVETQGKEPVTSRGYLEDEAAHAEMAFFNTILPTQAGARHDVTWYVSSSPCVTCAQRICEALRKNKGLRLTIMVGRLFMWEEPEMQAALRSMKEAGCKLRIMKPQDFEYVWKNFVEQEAKSFVPWEDIQENFQYYEEKLAE

SEQ ID NO: 597 - protein, 161 aa, synthetic construct
  REGION 1..161: note = Synthetic
FKFQFRNVEYSGRNKTFLCYVIETQGKESVTSRGYLEDEAAHAEIAFFNTILPKESSLRYNVTWYVSSSPCVTCADRITETLKKNKNLRLTIMVGRLFMWEEPEMQAALKKMKSAGCKLRIMKPQDFEYVWQNFVEQEAKAFVPWEDIQENFQYYEEKLAE

SEQ ID NO: 598 - protein, 161 aa, synthetic construct
  REGION 1..161: note = Synthetic
FKFQFRNVEYSGRNKTFLCYVVETQGKESATSRGYLEDEAAHAEMAFFNTILPKDSSLRYNITWYVSSSPCVTCAERIIETLKKNKNLRLTIMVGRLFMWEEPEMQAALKNMKSAGCKLRIMKPQDFEYVWQNFVEQEAKDFVPWEDIQENFNYYEEKLAE

SEQ ID NO: 599 - protein, 161 aa, synthetic construct
  REGION 1..161: note = Synthetic
FKFQFRNVEYSGRNKTFLCYVVETQGKESKTSRGYLEDEASHAEIAFFNTILPKESSLRYNITWYVSSSPCVTCADRISETLRKNKNLRLTIMVGRLFMWEEPEMQAALKKMKSAGCKLRIMKPQDFEYVWQNFVEQEAKAFVPWEDIQENFQYYEEKLAE

SEQ ID NO: 600 - protein, 154 aa, synthetic construct
  REGION 1..154: note = Synthetic
FKFQFRNVEYSGRNKTFLCYVVETQGKENQTARGYLEDEAAHAEEAFFNSILPADQALKYNVTWYVSSSPCAACADRIADTLRRTPNLRLLLLVGRLFMWEEPEIQAALKKLKAAGCKLRIMKPQDFEYVWQNFVEQEAKAFVPWEDIQENFLY

SEQ ID NO: 601 - protein, 161 aa, synthetic construct
  REGION 1..161: note = Synthetic
FKFQFRNVEYSGRNKTFLCYVVEVQGKQGQISRGYLEDEAAHAEEAFFKTILPTDPALRYNVTWYVSSSPCAACADRISSTLSKTKNLKMLLLVGRLFMWEEPEIKTALKKLKESGCKLRIMKPQDFEYVWQNFVEQESKAFVPWEDIQENFLYYEEKLAE

SEQ ID NO: 602 - protein, 161 aa, synthetic construct
  REGION 1..161: note = Synthetic
FKFQFRNVEYSGRNKTFLCYVLEVQGKQGQMSRGYLEDETAHAEEAFFKTILPTDPALRYNVTWYVSSSPCAACADRISSTLSKNKNLRMLLLVGRLFMWEEPEIKAALKRLKEAGCKLRIMKPQDFEYVWQNFVEQESKAFVPWEDIQENFLYYEEKLAE

SEQ ID NO: 603 - protein, 161 aa, synthetic construct
  REGION 1..161: note = Synthetic
FKFQFRNVEYSGRNKTFLCYVVEAQGKQVQASRGYLEDEAAHAEEAFFNTILPTDPSLRYNVTWYVSSSPCAACADRIIKTLGKTKNLRLLILVGRLFMWEEPEIQTALKKLKEAGCKLRIMKPQDFEYIWQNFVEQESKAFEPWEDIQENFLYYEEKLAD

SEQ ID NO: 604 - protein, 161 aa, synthetic construct
  REGION 1..161: note = Synthetic
FKFQFRNVEYSGRNKTFLCYVVEAQGKQVQASRGYLEDEAAHAEEAFFNTILPADPALRYNVTWYVSSSPCAACADRIIKTLSKTKNLRLLILVGRLFMWEELEIQDALKKLKEAGCKLRIMKPQDFEYVWQNFVEQESKAFQPWEDIQENFLYYEEKLAD

SEQ ID NO: 605 - protein, 161 aa, synthetic construct
  REGION 1..161: note = Synthetic
FKFQFRNVEYSGRNKTFLCYVVEAQGKQVQATRGYLEDEAAHAEEAFFNTILPADPALRYNVTWYVSSSPCAACADRITKTLSKTKNLRLLILVGRLFMWEEPEIQAALKKLKEAGCKLRIMKPQDFEYIWQNFVEQESKAFQPWEDIQENFLYYEEKLAD

SEQ ID NO: 606 - protein, 161 aa, synthetic construct
  REGION 1..161: note = Synthetic
FKFQFRNVEYSGRNKTFLCYVVEAQGKQVQASRGYLEDEAAHAEEAFFNTILPADPALRYNVTWYVSSSPCAACADRIIKTLNKTKNLRLLILVGRLFMWEEPEIQAALKKLKEAGCKLRIMKPQDFDYVWQNFVEQESKAFEPWEDIQENFQYYEEKLAD

SEQ ID NO: 607 - protein, 161 aa, synthetic construct
  REGION 1..161: note = Synthetic
FKFQFRNVEYSGRNKTFLCYVVEVQAKQVQASRGYLEDEAAHAEEAFFNTILPADPALKYNVTWYVSSSPCAACADHIIKTLNKTKNLRLLILVGRLFMWEEPEIQAALKKLKEAGCKLRIMKPQDFEYIWQNFVEQESKAFEPWEDIQENFLYYEEKLAD

SEQ ID NO: 608 - protein, 161 aa, synthetic construct
  REGION 1..161: note = Synthetic
FKFQFRNVEYSGRNKTFLCYVVEAQAKQVQATRGYLEDEAAHAEEAFFNTILPADPALRYNVTWYVSSSPCAACADRIIKTLSKTKNLRLLILVGRLFMWEEPEIQAALRKMREAGCKLRIMKPQDFEYIWQNFVEQESKAFEPWEDIQENFLYYEEKLAD

SEQ ID NO: 609 - protein, 161 aa, synthetic construct
  REGION 1..161: note = Synthetic
FKFQFRNVEYSGRNKTFLCYVVEAQSKQVQATRGYLEDEGAHAEEAFFNTILPADPALKYNVTWYVSSSPCAACADRILKTLSKAKNLRLLILVGRLFMWEEPEVQAALKKLKEAGCKLRIMKPQDFEYIWQNFVEQESKAFEPWEDIQENFLYYEEKLAD

SEQ ID NO: 610 - protein, 161 aa, synthetic construct
  REGION 1..161: note = Synthetic
FKFQFRNVEYSGRNKTFLCYVVEVQSKQAQATQGYLEDEGAHAEEAFFNTILPADPALKYNVTWYVSSSPCAACADRILKTLSKTKNLRLLILVSRLFMWEEPEVQAALKKLKEAGCKLRIMKPQDFEYIWQNFVEQESKAFEPWEDIQENFLYYEEKLAD

SEQ ID NO: 611 - protein, 161 aa, synthetic construct
  REGION 1..161: note = Synthetic
FKFQFRNVEYSGRNKTFLCYVVEAQSKQVQASRGYLEDEAAHAEEAFFNTIMPADPALRYNVTWYVSSSPCAACADRIIKTLSKTKNLRLLILVGRLFMWEEPEIQAALKKLKEAGCRLRIMKPQDFEYVWQNFVEQESKTFEPWEDIQENFLYYEEKLAD

SEQ ID NO: 612 - protein, 161 aa, synthetic construct
  REGION 1..161: note = Synthetic
FKFQFRNVEYSGRNKTFLCYVVEAQSKQVQASRGYLEDETNHAEEAFFNSIMPTDPALRYMVTWYVSSSPCAACADRIVKTLNKTKNLRLLILVGRLFMWEEPEIQAALRKLKEAGCRLRIMKPQDFEYIWQNFVEQESKAFEPWEDIQENFLYYEEKLAD

SEQ ID NO: 613 - protein, 161 aa, synthetic construct
  REGION 1..161: note = Synthetic
FKFQFRNVEYSGRNKTFLCYVVEAQSKQVQATRGYLEDEAAHAEEAFFNTIMPTDPALRYIVTWYVSSSPCAACADRIIKTLNKTKNLRLLILVGRLFMWEEPEIQAALRKLKEAGCKLRIMKPQDFEYVWQNFVEQESKAFEPWEDIQENFLYYEEKLAD

SEQ ID NO: 614 - protein, 161 aa, synthetic construct
  REGION 1..161: note = Synthetic
FKFQFRNVEYSGRNKTFLCYVIEAQSKQVQATRGYLEDEAAHAEEAFFNTILPADPAVRYVVTWYVSSSPCAACADRIIKTLNKAKNLRLLILVGRLFMWEEPEIQAALRRLKEAGCRLRIMKPQDFEYVWQNFVEQESKAFEPWEDIQENFLYYEEKLAD

SEQ ID NO: 615 - protein, 161 aa, synthetic construct
  REGION 1..161: note = Synthetic
FKFQFRNVEYSGRNKTFLCYVVEVQSKQVQASRGYLEDEAAHAEEAFFNSVLPADPALRYNVTWYVSSSPCAACADRIIKTLGKTKNLRLLILVGRLFMWEEPEIQAALRKLKEAGCRLRIMKPQDFEYVWQNFVEQESKAFEPWEDIQENFLYYEDKLAD

SEQ ID NO: 616 - protein, 161 aa, synthetic construct
  REGION 1..161: note = Synthetic
FKFQFRNVEYSGRNKTFLCYVVEVQSKQVQASRGYLEDEAAHAEEAFFNTILPADPALRYSVTWYVSSSPCVACADRIVKTLGKTKNLRLLILVGRLFMWEEPEIQAALRKLKEAGCRLRIMKPQDFEYVWQNFVEQESKAFEPWEDIQENFLYYEEKLAD

SEQ ID NO: 617 - protein, 161 aa, synthetic construct
  REGION 1..161: note = Synthetic
FKFQFRNVEYSGRNKTFLCYVVEVQCKQVQASRGYLEDEAAHAEEAFFNTILPADPALRYNVTWYVSSSPCVACADRIIKTLAKTKNLRLLILVGRLFMWEEPEMQAALRKLKEAGCRLRIMKPQDFEYVWQNFVEHESKAFEPWEDIKENFLYYEEKLAD

SEQ ID NO: 618 - protein, 161 aa, synthetic construct
  REGION 1..161: note = Synthetic
FKFQFRNVEYSGRNKTFLCYVIEVQDKQVQASRGYLEDETAHAEEAFFNTVVPTDPALRYNVTWYVSSSPCVACADRIIKMLSKTKNLRLLILVGRLFMWEEPAMQAALKKLKEAGCRLRIMKPQDFEYIWQNFVEQESKAFEPWEDIQENFLYYEEKLAD

SEQ ID NO: 619 - protein, 161 aa, synthetic construct
  REGION 1..161: note = Synthetic
FKFQFRNVEYSGRNKTFLCYVVEVQGKQVQATRGYLEDEAAHAEEAFFNTILPADPALRYNVTWYVSSSPCAACADRIIRTLGKTKNLRLLILVGRLFMWEEPEIQAALKKLREAGCRLRIMKPQDFEYVWQNFVEQEAKAFEPWEDIQENFLYYEEKLAD

SEQ ID NO: 620 - protein, 161 aa, synthetic construct
  REGION 1..161: note = Synthetic
FKFQFRNVEYSGRNKTFLCYVVEAQGKQVQASRGYLEDEAAHAEEAFFNTILPADPAVRYNVTWYVSSSPCAACADRIVRTLGKTKNLRLLILVGRLFMWEEPDVQAALRRLKEAGCRLRIMKPQDFEYVWQNFVEQESKAFEPWEDIQENFLYYEEKLAD

SEQ ID NO: 621 - protein, 161 aa, synthetic construct
  REGION 1..161: note = Synthetic
FKFQFRNVEYSGRNKTFLCYVVEAQGKQVQATRGYLEDEAAHAEEAFFNTILPADPALRYTVTWYVSSSPCAACADRIARTLGQTKNLRLLILAGRLLLWEEPDVRAALRRLAEAGCRLRVMKPQDFEYVWQQFVEQEPKAFEPWEDIQENFLYYEEKLAD

SEQ ID NO: 622 - protein, 161 aa, synthetic construct
  REGION 1..161: note = Synthetic
FKFQFRNVEYSGRNKTFLCYVVETQAKQVQATRGYLEDETNHAEEAFFNTILPSDPALRYNVTWYVSSSPCAACADRILRTLGKTKNLRLLILVGRLFMWEEPEVQAALRKLKEAGCRLRIMKPQDFEYVWQNFVEQESKAFEPWGDIQENFLYYEEKLAD

SEQ ID NO: 623 - protein, 161 aa, synthetic construct
  REGION 1..161: note = Synthetic
FKFQFRNVEYSGRNKTFLCYVVEAQGKQVQASRGYLEDEAAHAEEAFFNTILPTDPALRYNVTWYVSSSPCAACADRIIRTLGKTKNLRLLILVGRLFMWEEPEVQAALRKLKEAGCRLRIMKPQDFEYVWQNFVEQESKAFQPWEDIQENFLYYEEKLAD

SEQ ID NO: 624 - protein, 163 aa, synthetic construct
  REGION 1..163: note = Synthetic
FYFHFKNCPDHGRNGCYLCYEVKRQRGLPLVGTGVFENEPKHTEICFLNWFKTQQNLSREEKYHVTWFMSWSPCFQCARQVVEFLKDHEYVQLSIFVARLYYSSRPEYQQGLRSLQGAGAQVAIMTPDDFAYCRKVFVHDHKPFRYWKGIYINSCSLSKTLED

SEQ ID NO: 625 - protein, 158 aa, synthetic construct
  REGION 1..158: note = Synthetic
FNSNFKNLDGGCKSTFLCFEVEREDGSVLYQNGVFRNQHAELCFIEWFHEKVLCPDAQYHVTWYISWSPCFECAEQVADFLNENENVDLSISAARLYLCEDEDEQGLQDLVATGAKVAMMAPEDFKYCWDNFVYNGWQFTYWKNVRRNYGRLQEKLDE

SEQ ID NO: 626 - protein, 115 aa, synthetic construct
  REGION 1..115: note = Synthetic
NSCSLSKTEDILRHAELCFLDWFREKVLCPDAQYHVTWYISWSPCFECAEQVADFLNENENVDLSISAARLYLCEDEDEQGLQDLVATGAKVAMMAPEDLTAKMVPDETPMFDPS

SEQ ID NO: 627 - protein, 161 aa, synthetic construct
  REGION 1..161: note = Synthetic
FSFHFKNLMFAGRNCTYLCYQVKREHCSPVPDKGVFQNEPCHAELCFLSWFNKRLSPDECYHITWFMSWSPCFACTEQVAKFLEKNRNVRLSIFAARLYYFWQPAVQQGLRRLHGVGACVGIMSYQDFKYCWENFVYNRMPFKPWEKQCENSKILVTKLEE

SEQ ID NO: 628 - protein, 159 aa, synthetic construct
  REGION 1..159: note = Synthetic
FSFHFRNLEFAGRNCSYICYRVEGLSGSPGSEQGVFLNECRHAELCFLHWFRGRLSPDEYYHVTWFISWSPCSNCAREVAEFLKRHRNVELSIFAARLYYWQRNKPDLRNLCSSGAQLAIMFYQDFRYCWDNFVHNGREFIPWEKINVNSRLLATNLEE

SEQ ID NO: 629 - protein, 143 aa, synthetic construct
  REGION 1..143: note = Synthetic
FSFHFRNLKFAGRKCSYLCYRVEGLSGSPGSEQGVFLNERRHAELCFLDWFRVRLSPDEYYRVTWFISWSPCSYCAREVADFLKQYRNVKLSIFAARLYYCRDHAQGLRSLCSSGAQLAIMFFWDFRYCWDNFVHNGRDFIPW

SEQ ID NO: 630 - protein, 162 aa, synthetic construct
  REGION 1..162: note = Synthetic
FYFQFCNLLYARRNCSYICYKVERKYHSRAFDWGVFHNQRCHTELRFLSWFHAEKLRPNERYHITWFMSWSPCMKCAKEVADFLGRHQNVTLSIFTARLYNFQEEGSRQGLLRLSDQGAHVDIMSYQEFKYCWKKFVNSRRPFRPWKKLYRNYQRLVEELED

SEQ ID NO: 631 - protein, 162 aa, synthetic construct
  REGION 1..162: note = Synthetic
FFFEFQNLLYAGRKSSYLCFQVERQHSSPVSDWGVFENQPYHAELCFLNWFRAEKLSPYEHYDVTWFLSWSPCSTCAEEIAIFLSNHKNVRLSIFVSRIYYFWKPAFRQGLQELDHLGVQLDAMSFDDFKYCWENFVDNGMPFRCWKKVHRNYKFVLRKLNE

SEQ ID NO: 632 - protein, 161 aa, synthetic construct
  REGION 1..161: note = Synthetic
FRFHFPNLLYAGRKLCYLCFQVETDYFSCDSDRGVFRNKRCHAEQCFLSWFRDQYPCRDEYYNVTWFLSWSPCPTCAEEVVEFLEEYRNLTLSIFTSRLYYFYHPNYQQGLRKLWDAGVQLDIMSCDDFEHCWDNFVDHGMRFQRRNLLKDYDFLAAELQE

SEQ ID NO: 633 - protein, 158 aa, synthetic construct
  REGION 1..158: note = Synthetic
FFFQFPSLCYAGRKFCYLCFQVGRGHPSDWGVFRNKPYHAESCFLSWFRAQNLSPDEDYHVTWFSSWSPCHTCADEVVEFLGQYRHVTLSIFAARLYYFWDPPFQNGLRRLQSAGVRLDIMSFADYKRCWENFVDHGMRFQSRNLLRHRDLLASRLEN

SEQ ID NO: 634 - protein, 161 aa, synthetic construct
  REGION 1..161: note = Synthetic
FYFHFQNLLFAGRNTTFLCCRVDKERHGTVLVSGVFTHQYHAESHFLLWFQKNFLSLDKDFQVTWYLSWSPCPACAKQVADFLAVHRNVSLTIFSARLYYFWDPEFRDGLHRLFEKGARVAIMSPKDFENCWEGFVFNGRDFRPWDNMVENYQSLRITLQE

SEQ ID NO: 635 - protein, 162 aa, synthetic construct
  REGION 1..162: note = Synthetic
FLFHFRNLRWAGRNNTFLCYQVDRERDSTVIHRGVFKTQRLHAELCFLYWLHDYPLFPDQHFHITWFISWSPCSDCAQQVAAFLASHSNLSLTVYTARLYYFWKHSYQEGLRALQREGARVEIMSIREFEHCWENFVYPGRPFRPWKNLFRNYYFQVKKLQK

SEQ ID NO: 636 - protein, 162 aa, synthetic construct
  REGION 1..162: note = Synthetic
FYFHFKNLRYALRKNTFLCYEVNRECNELVLCQGVFRKENLHAEVCFLYWFHTQVLPPDEKYKITWYVSWSPCNECAEKVASFLDTHRNLSLAIFSSRLYYFWDPDYQDKLRRLNQAGAQIAAMDFPEFEKCWNKFVDNGKSFRPWKRLKINFRFQDNKLRD

SEQ ID NO: 637 - protein, 162 aa, synthetic construct
  REGION 1..162: note = Synthetic
FYFHFKNVRYAGRKNNFLCYEVNGDCALPVLRQGVFRKQHIHAELCFIYWFHDKVLSPMEEFKVTWYMSWSPCSKCAEQVARFLAAHRNLSLAIFSSRLYYYLNPNYQQKLCRLIQEGVHVAAMDLPEFKKCWNKFVDNGQPFRPWMRLRINFSFYDCKLQE

SEQ ID NO: 638 - protein, 162 aa, synthetic construct
  REGION 1..162: note = Synthetic
FKFHFKNLRYADRKDTFLCYEVTRDCDSPVLHHGVFKNKNIHAEICFLYWFHDKVLSPREEFKITWYMSWSPCFECAEQVLRFLATHHNLSLDIFSSRLYNIRDPENQQNLCRLVQEGAQVAAMDLYEFKKCWKKFVDNGRRFRPWKKLLTNFRYQDSKLQE

SEQ ID NO: 639 - protein, 162 aa, synthetic construct
  REGION 1..162: note = Synthetic
FKFHFKNLGYAGRKDTFLCYEVTRDCDSPVLHHGVFKNKNIHAEICFLYWFHDKVLSPREEFKITWYMSWSPCFECAEQIVRFLATHHNLSLDIFSSRLYNVQDPETQQNLCRLVQEGAQVAAMDLYEFKKCWKKFVDNGRRFRPWKRLLTNFRYQDSKLQE

SEQ ID NO: 640 - protein, 162 aa, synthetic construct
  REGION 1..162: note = Synthetic
FYFHFENLQKAGRNETWLCFAVEIKQHSTVWKTGVFRNQHCHAERCFLSWFCDNTLSPKKNYQVTWYISWSPCPECAGEVAEFLATHSNVKLTIYTARLYYFWDTDYQEGLRSLSEEGASMEIMGYEDFKYCWENFVYNGEPFKPWKGINTNFRFLERRLWK

SEQ ID NO: 641 - protein, 162 aa, synthetic construct
  REGION 1..162: note = Synthetic
FYFHFKNLRTADRNETWLCFAVEIKQRSTVWRTGVFRNQHCHAERCFLSWFRDNPLSPKKNYQVTWYTSWSPCPECAGEVAEFLARYSNVQLTIYTARLYYFWDTDYQEGLRSLSEEGASVEIMGYEDFKYCWENFVCDGEPFKPWKGINTNFRFLERHLRK

SEQ ID NO: 642 - protein, 162 aa, synthetic construct
  REGION 1..162: note = Synthetic
FYFHFKNLRKAGRNESWLCFTMEVKHHSPVWKRGVFRNQHCHAERCFLSWFCDDILSPNTNYQVTWYTSWSPCPECAGEVAEFLARHSNVNLTIFTARLYYFWDTDYQEGLRSLNQEGASVKIMGYKDFKYCWENFVYNDEPFKPWKGLKYNFLFLDSKLQE

SEQ ID NO: 643 - protein, 162 aa, synthetic construct
  REGION 1..162: note = Synthetic
FYFHFKNLRKAGRNESLLCFTMEIKQCSTVWKRGVFRNQHCHAERCFLSWFCEDILSPNTDYQVTWYTSWSPCLDCAGEVAEFLARHSNVKLAIFAARLYYFWDTDYQQGLRSLSEEGASVQIMGYEDFKYCWENFVYNDEPFKPWKGLKYNFLFLDSKLQE

SEQ ID NO: 644 - protein, 162 aa, synthetic construct
  REGION 1..162: note = Synthetic
FYFHFKNLRKAGRNESWLCFTMEIKQCSTVWKRGVFRNQRCHAERCFLSWFCEDILSPNTDYQVTWYTSWSPCLDCAGEVAEFLARHSNVKLTIYTARLYYFWHTDYQQGLRSLSEEGASVEIMGYEDFKCCWENFVYNDEPFKPWKGLKYNFLFLDSKLQE

SEQ ID NO: 645 - protein, 162 aa, synthetic construct
  REGION 1..162: note = Synthetic
FYFQFKNLWEADRNETWLCFTVEGKRRSVVWKTGVFRNQHCHAERCFLSWFCDDILSPNTNYQVTWYTSWSPCPECAGEVAEFLARHSNVNLTIFTARLYYFWDTDYQEGLRSLNQEGASVKIMGYKDFKYCWENFVYNDEPFKPWKGLKYNFLFLDSKLQE

SEQ ID NO: 646 - protein, 162 aa, synthetic construct
  REGION 1..162: note = Synthetic
FYFQFKNLWEADRNETWLCFTVEGKRRSVVWKTGVFRNQHCHAERCFLSWFCDDILSPNTNYEVTWYTSWSPCPECAGKVAEFLARHSNVNLTIFTARLCYFWDTDYQEGLRSLNQEGASVKIMGYKDFVSCWKNFVYSDEPFKPWKGLQTNFRLLKRRLRE

SEQ ID NO: 647 - protein, 162 aa, synthetic construct
  REGION 1..162: note = Synthetic
FYDNFENEPILGRSYTWLCYEVKIRGRSNLWDTGVFRGEHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDEPFKPWKGLQTNFRLLKRRLRE

SEQ ID NO: 648 - protein, 162 aa, synthetic construct
  REGION 1..162: note = Synthetic
FYFHFKNLLKAGRNESWLCFTMEVKHHSAVRKRGVFRNQHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDEPFKPWKGLQTNFRLLKRRLRE

SEQ ID NO: 649 - protein, 162 aa, synthetic construct
  REGION 1..162: note = Synthetic
FYFQFKNLWEADRNETWLCFTVEGKRRSVVWKTGVFRNQHCHAERCFLSWFCDDILSPNTNYQVTWYTSWSPCPECAGEVAEFLARHSNVNLTIFTARLYYFQDTDYQEGLRSLSQEGVAVKIMDYKDFKYCWENFVYNDEPFKPWKGLKYNFRFLKRRLQE

SEQ ID NO: 650 - protein, 162 aa, synthetic construct
  REGION 1..162: note = Synthetic
FYFQFKNLWEADRNETWLCFTVEGKRRSVVWKTGVFRNQHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNNEPFKPWKGLKTNFRLLKRRLRE

SEQ ID NO: 651 - protein, 162 aa, synthetic construct
  REGION 1..162: note = Synthetic
FYFQFKNLWEADRNETWLCFTVEVKHHTVVWKRGVFRNQHCHAESCFLSWFCNNILSPNTNYRVTWYASWSPCPECAGEVAKFLARHSNVKLTIFTARLYYFQNPYYQQGLRRLSQEGVAVGIMDYEDFKDCWENFVYSDEPFKPWKGINTNFRLLKKRLRE

SEQ ID NO: 652 - protein, 162 aa, synthetic construct
  REGION 1..162: note = Synthetic
FYFQFKNLWEANRNETWLCFTVEVKQRSTVWERGVFQNQHCHAERCFLSWFCEDILSPNTDYQVTWYTSWSPCPECAGEVAEFLARHNNVMLTIYTARLYYSQDPNYQQGLRSLSEKGVSVKIMDYEDFKYCWENFVYNDEPFKPWKGLKYNFLFLDSKLQE

SEQ ID NO: 653 - protein, 162 aa, synthetic construct
  REGION 1..162: note = Synthetic
FYFQFKNLREANRNETWLCFTVEGRHHLTVWKTGVFRNQHCHAEKCFLSWFCKNILSPNTDYQVTWYTSWSPCPECAREVAKFLARHNNVMLTIYTARLYYSQCPNYQQGLRSLSEKGVSVEIMDYEDFKYCWQNFVYDGEPFKPWKGLKTSFRFLKRCLRE

SEQ ID NO: 654 - protein, 162 aa, synthetic construct
  REGION 1..162: note = Synthetic
FYFQFKNLREANRNETWLCFAVEIKQRSTVLKTGVFRNQHCHAERCFLSWFCEDILSPNTDYQVTWYTSWSPCLDCAGEVAKFLARHNNVMLTIYTARLYYSQYPNYQQGLRSLSEKGVSVKIMDYEDFKYCWEKFVYDGEPFKPWKGLKTSFRFLKRRLRE

SEQ ID NO: 655 - protein, 120 aa, synthetic construct
  REGION 1..120: note = Synthetic
LHAELSFLSWFHDTELSFDENYKVTWYMSWSPCPECAKEIVTFLDNHHNVTLTIYVARLYYHWNPTYKEGLRALVQGGTRLYTMAFPEFEDCWSLFVNETFRPWENFHKCCSLQDKTLQK

SEQ ID NO: 656 - protein, 158 aa, synthetic construct
  REGION 1..158: note = Synthetic
FYFHFENLPYAGRNKTFLCYEVKRRDNKLHKGVVQNQLSRTELSFISCFHATELCLDETYKVTWYISWSPCVECAEEIVKFLANHRNVFLTVFIARLYYYREHTFKEGLQALDNGGVQMHMMCLQDFKDCWSLFVSETFRPWKGLRKYYLFQNKTLKQ

SEQ ID NO: 657 - protein, 157 aa, synthetic construct
  REGION 1..157: note = Synthetic
FYFHFENLPNAGRHKTFLCFEVKNNELHKGFFLNQLHAELRFLSWLHDTCLCPYEYYQVTWYMSWSPCVECAEELTTFLAGHRNVTLTIYVARLYYHQFPVYKNRLQALIKKGATVKVMFFRDFLYCWRRFVYNFKRFYDWPNLHKNSLHYYKTLQH

SEQ ID NO: 658 - protein, 160 aa, synthetic construct
  REGION 1..160: note = Synthetic
FYFHFENLPDPGWNKTFLCYEVKRQDQKLRKGVFQNQPLHAELRFLSWFHDTLLCPLGSYQVTLYVSWSPCSECAEELTTFLAGHRNVTMTIYVAQLYYCNKSPNREGLKILIAEDARLRVMFYDEFLYCWRNFVKNYNNFDPWSLLDENSRYHNRILQN

SEQ ID NO: 659 - protein, 160 aa, synthetic construct
  REGION 1..160: note = Synthetic
FRFYFRNLRCAGRNKTFLCYEVKRRDNKLHKGVVLNQPLHAELRFLSWFHDTLLCPLGSYQVTLYVSWSPCSECAEELTTFLAGHRNVTMTIYVAQLYYCNKSPNREGLKILIAEDARLRVMFYDEFLYCWRNFVKNYNNFDPWSLLDENSRYHNRILQN

SEQ ID NO: 660 - protein, 160 aa, synthetic construct
  REGION 1..160: note = Synthetic
FKFHFENLPNPGWHKTFLCYTIESQKNRLRNGVFQNQRLHAELRFLSWFHDNWLCPGNSYRVTFYMSWSPCSECAEELTTFLAGHRNVTLTIFFSKLYYCDDSSNREGLKTLAAGGARLFVMFDTDFSYCWTNFVNCYNYFEPWPLLDDNSKYCNRILQK

SEQ ID NO: 661 - protein, 160 aa, synthetic construct
  REGION 1..160: note = Synthetic
FYFHFNNLCFAGRNKTFLCYTIESKKNRLRNGVFQNQRLHAELRFLSWFHDNWLCPGNSYQVTLYMSWSPCSECAEELTTFLAGHRNVTLTIFFSKLYCCDDSPHREGLKTLAAGGARLFVMFDTDFSYCWTNFVNCYNYFEPWPLLDDNSKYYKSILQK

SEQ ID NO: 662 - protein, 158 aa, synthetic construct
  REGION 1..158: note = Synthetic
FNCHFNNRPYLRRNDTWLCFEVKTSSNSPGFYSGVFQNQPWHTELCILTWARPMLSHHHFYQITWYMSWSPCANCAWQVAAFLATHENVSLTIYTAHIYYMWRQDYRQGMLRMIEEGTRVYIMFSKEFQHCWENFVDHGMCWNRVKKNYEFLVTQLNE

SEQ ID NO: 663 - protein, 149 aa, synthetic construct
  REGION 1..149: note = Synthetic
FYNQFNNTPVPGRTDTWLCFEVKNNSNSPGFHRGSSENRHAEVGFLTWFQKEMPPNHHYEVTWYISWSPCVHCAWHVVNFLTSNPNMTLTIFAARLYYIYHPEEGTKVHIVSLKEFKYCWAKLVYNGMRFMPWYQFNENYQLLVTQLKK

SEQ ID NO: 664 - protein, 144 aa, synthetic construct
  REGION 1..144: note = Synthetic
FYYNFENRSVLGWNTTWLCHKVKTKDPSKLLDTRIFGGQHHPEMRFLDWFCKYISRSPCPEYAEKVAEFLVKNGKVTLTIFVAHLYYFWEPDYQNGVRPHASMTIMNYDEFQHCWDKFVYNRMLFDPWKQLNTNYALLHSMLGE

SEQ ID NO: 665 - protein, 122 aa, synthetic construct
  REGION 1..122: note = Synthetic
QHHPEMRFLHWFRKWKLHSDQEYEVTWFVSWSPCPVCARNVAEFLTEDGKVTLTIFVARLYYFWIPHYREELRRLCQPRATMKIMSYGEFQHCWDKFVDNRLYKPWNKLPKHYTLLHITLGE

SEQ ID NO: 666 - protein, 163 aa, synthetic construct
  REGION 1..163: note = Synthetic
FFYNFNNRPILRRNTVWLCYEVKTRGPSMPWDAKIFRGQKYHPEMRFLHWFRKWRQLHRDQEYEVTWYVSWSPCTGCANSVATFLAEDPKVTLTIFVARLYYFWKPDYQEALRVLCQPHATMKIMNYNEFQHCWNKFVRGREPFEPWENLPKHYTLLHATLGE

SEQ ID NO: 667 - protein, 163 aa, synthetic construct
  REGION 1..163: note = Synthetic
FFYNFNNRPILRRNTVWLCYEVKTRGPSVPWGTKIFRGQKYHPEMRFLRWFHKWRQLHHDQEYKVTWYVSWSPCTRCANSVATFLAKDPKVTLTIFVARLYYFWKPDYQQALRILCQPHATMKIMNYNEFQDCWNKFVDGGKPFKPRNNLPKHYTLLQATLGE

SEQ ID NO: 668 - protein, 163 aa, synthetic construct
  REGION 1..163: note = Synthetic
FVYYFNNRPILGRNIVWLCCEVKTKDPSGPLDANIFQGEKDHPEMKFLHWFRKWRQLHRDQEYEVTWYVSWSPCTRCANSVATFLAEDPKVTLTIFVARLYYFWKPHYQEALRILCQPHATMKIMNYNEFQHCWNEFVDGGKPFKPRKNLPKHYTLLHATLGE

SEQ ID NO: 669 - protein, 163 aa, synthetic construct
  REGION 1..163: note = Synthetic
FVSNFNNRPILGLNTVWLCCEVKTKDPSGPLDAKIFQGKKYHPEMRFLRWFHKWRQLHHDQEYKVTWYVSWSPCTRCANSVATFLAKDPKVTLTIFVARLYYFWKPDYQQALRILCQPHATMKIMNYNEFQDCWNKFVDGGKPFKPRNNLPKHYTLLQATLGE

SEQ ID NO: 670 - protein, 160 aa, synthetic construct
  REGION 1..160: note = Synthetic
FSYNFKNRPILRRNTVWLCYEVKTKGPSRPLDAKIFRGQKNHPEMRFFHWFSKWRTLHRDQECEVTWYMSWSPCTKCTRNVATFLAEDPKVTLTIFVARLYYFWDPDYQEALRSLCRPRANMKIMNYDEFQHCWNKFVYSRELFEPWNNLPKYYIPLHKV

SEQ ID NO: 671 - protein, 162 aa, synthetic construct
  REGION 1..162: note = Synthetic
FSYNFYNRPILRRNTVWLCYEVKTKGPSRPLDAKIFRGQKYHPEMRFFHWFSKWRKLHRDQEYEVTWYISWSPCTKCTRDMATFLAEDPKVTLTIFVARLYYFWDPDYQEALRSLCQPRATMKIMNYDEFQHCWSKFVYSRELFEPWNNLPKYYILLHIMLG

SEQ ID NO: 672 - protein, 162 aa, synthetic construct
  REGION 1..162: note = Synthetic
FSYNFNNRPILRRNTVWLCYEVKTKDPSRPLDAKIFRGQKYHPEMRFFHWFSKWRKLHRDQEYEVTWYISWSPCTKCTRNVATFLAEDPKVTLTIFVARLYYFWDQDYQEALRSLCQPRATMKIMNYDEFQHCWSKFVYSRELFEPWNNLPKYYMLLHIMLG

SEQ ID NO: 673 - protein, 162 aa, synthetic construct
  REGION 1..162: note = Synthetic
FSYNFNNRPILRRNTVWLCYEVKTKGPSRPLDAKIFRGQQYHAEMCFLSWFCGNQLPAYKCFQITCFVSWTPCPDCVAKLAEFLAEHPNVTLTISAARLYYYWERDYRRALRRLRQAGARVKIMDDEEFAYCWENFVYSGQPFMPWHKFDDNYAFLHRTLKE

SEQ ID NO: 674 - protein, 162 aa, synthetic construct
  REGION 1..162: note = Synthetic
FSYNFYNRPILRRNTVWLCYEVKTKGPSRPLDAKIFRGQEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSGQPFMPWYKFDDNYAFLHRTLKE

SEQ ID NO: 675 - protein, 146 aa, synthetic construct
  REGION 1..146: note = Synthetic
WFCFEVKTRGPSMPWDAKIFRGQQYHAEMCFLSRFCGNQLPAYKRFQITWFVSWTPCPDCVVKVAEFLAEHPNVTLTISAARLYYYWETDYRRALCRLRQAGAHVKIMDNEEFAYCWENFVYNGQSFMPWDKFDDNYAFLHCKLKE

SEQ ID NO: 676 - protein, 162 aa, synthetic construct
  REGION 1..162: note = Synthetic
FYYHFENKPILGRSYTWLCYEVKIKDPSKLWYTGVFRGQEHHAEMCFLSRFCGNQLPAYKRFQITWFVSWNPCPDCVAKVIEFLAEHPNVTLTISTARLYYYWGRDWQRALCRLRQAGARVKIMDYEEFAYCWENFVYNGQSFMPWDKFDDNYAFLHCKLKE

SEQ ID NO: 677 - protein, 123 aa, synthetic construct
  REGION 1..123: note = Synthetic
EHHAEMCFLSRFCGNQLPAYKRFQITWFVSWNPCPDCVAKVIEFLAEHPNVTLTISTARLYYYWGRDWQRALCRLRQAGARVKIMDYEEFAYCWENFVYNDQSFMPWYKFNDNYAFLHRMLKE

SEQ ID NO: 678 - protein, 162 aa, synthetic construct
  REGION 1..162: note = Synthetic
FYYNFENEPILGRRYTWLCYEVKIKDPSKLWDTGVFPGQQYHAEMYFLSWFCGNQLPAYKHFQITWFVSWNPCPDCVAKVTEFLAEHRNVTLTISAARLYYYWGKDWRRALCRLHQAGARVKIMDDEEFAYCWENCVYNGQPFMPWDKFDDNYAFLHLKLKE

SEQ ID NO: 679 - protein, 162 aa, synthetic construct
  REGION 1..162: note = Synthetic
FDYNFENEPILGRSYTWLCYEVKIEDPSKLWDTGVFQGQQYHAEMCFLSRFCGNQLPAYKRFQITWFVSWNPCPDCVAKVTEFLAEHPNVTLTISAARLYYYWGRDWRRALRRLHQAGARVKIMDYEEFAYCWENFVYNGQSFMPWDKFDDNYAFLHCKLKD

SEQ ID NO: 680 - protein, 162 aa, synthetic construct
  REGION 1..162: note = Synthetic
FNYNFENEPILGRSYTWLCYEVKIKDPSKLWDTGVFRGQQYHAEMCFLSWFCGNQLPAYKRFQITWFVSWNPCPDCVAKVTEFLAEHPNVTLTISAARLYYYWGKDWRRALRRLHQAGALVKIMDYEEFAYCWENFVYNGQSFMPWDKFDDNYASLHCKLKE

SEQ ID NO: 681 - protein, 162 aa, synthetic construct
  REGION 1..162: note = Synthetic
FYYHFENEPILGRSYTWLCYEVKIKDPSKLWDTGVFRGQEHHAEMCFLSWFCGNQLPAHKRFQITWFVSWTPCPDCVAKVAEFLAEYPNVTLTISAARLYYYWETDYRRALCRLRQAGARVKIMDYEEFAYCWENCVYNGQPFMPWYKFDDNYAFLHHKLKE

SEQ ID NO: 682 - protein, 162 aa, synthetic construct
  REGION 1..162: note = Synthetic
FYYNFENEPILRRSYTWLCYEVKIKDPSKLWDTGVFRGQEYHAEMCFLSWFCGNQLPAYKRFQITWFVSWTPCPDCVAKVAVFLAEHPNVTLTISAARLYYYWEKDWQRALCRLSQAGARVKIMDYEEFEYCWENFVYNGEPFMPWYKFDDNYAFLHHTLKE

SEQ ID NO: 683 - protein, 162 aa, synthetic construct
  REGION 1..162: note = Synthetic
FYYDFENEPILRRNYTWLCYEVKIKDPSKLWDTGVFRGQEHHAEMCFLSWFCGNQLSAYERFQITWFVSWTPCPDCVAKLAEFLAEHPNVTLTVSAARLYYYWERGYRRALRRLRQAGAHVKIMDYEEFAYCWENFVYNGQPFMPWYKFDDNYAFLHHTLKE

SEQ ID NO: 684 - protein, 162 aa, synthetic construct
  REGION 1..162: note = Synthetic
FYYNFENEPILGRSYTWLCYEVKIRGHSNLWDTGVFRGQEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNGQPFMPWYKFDDNYAFLHRTLKE

SEQ ID NO: 685 - protein, 162 aa, synthetic construct
  REGION 1..162: note = Synthetic
FYDNFENEPILGRSYTWLCYEVKIRGRSNLWDTGVFRGPENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNGQPFMPWYKFDDNYASLHRTLKE

SEQ ID NO: 686 - protein, 162 aa, synthetic construct
  REGION 1..162: note = Synthetic
FYDNFENEPILGRSYTWLCYEVKIRGRSNLWDTGVFRGQQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNGQQFMPWYKFDENYAFLHRTLKE

SEQ ID NO: 687 - protein, 162 aa, synthetic construct
  REGION 1..162: note = Synthetic
FYDNFENEPILGRSYNWLCYEVKIRGRSNLWNTGVFRGQEHHAEMCFLSWFCGNQLPAYKCFQITWFVSWTPCPDCVAKLAEFLAEYPNVTLTISTARLYYYWERDYRRALCRLSQAGARMKIMDYEECAYCWENFVYKGQQFMPWYKFDENYAFLHHTLKE

SEQ ID NO: 688 - protein, 1824 aa, synthetic construct
  REGION 1..1824: note = Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRK

SEQ ID NO: 689 - protein, 1838 aa, synthetic construct
  REGION 1..1838: note = Synthetic
MHHHHHHHHGSGGSGSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRK

SEQ ID NO: 690 - protein, 1789 aa, synthetic construct
  REGION 1..1789: note = Synthetic
MHHHHHHHHGSGGSGSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 691 - protein, 1775 aa, synthetic construct
  REGION 1..1775: note = Synthetic
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSPKKKRKV

SEQ ID NO: 692 - RNA, 43 nt, synthetic construct
  misc_feature 1..43: note = Synthetic
  source 1..43: mol_type = other RNA
gttttagagctagaaatagcaagttaaaataaggctagtccgt

SEQ ID NO: 693 - protein, 1853 aa, synthetic construct; identical to SEQ ID NO: 12
  REGION 1..1853: note = Synthetic

SEQ ID NO: 694 - protein, 1803 aa, synthetic construct
  REGION 1..1803: note = Synthetic
MKRTADGSEFESPKKKRKVSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKV

(19) United States
(12) Patent Application Publication
(10) Pub. No.: US 2025/0059244 A1
(43) Pub. Date: Feb. 20, 2025

(54) BASE EDITORS AND USES THEREOF

(71) Applicants: The Broad Institute, Inc., Cambridge, MA (US); President and Fellows of Harvard College, Cambridge, MA (US)

(72) Inventors: David R. Liu, Cambridge, MA (US); Luke W. Koblan, Cambridge, MA (US); Christopher Gerard Wilson, Cambridge, MA (US); Jordan Leigh Doman, Cambridge, MA (US)

(73) Assignees: The Broad Institute, Inc., Cambridge, MA (US); President and Fellows of Harvard College, Cambridge, MA (US)

(21) Appl. No.: 18/791,223

(22) Filed: Jul. 31, 2024

Related U.S. Application Data
(62) Division of application No. 17/057,398, filed on Nov. 20, 2020, now Pat. No. 12,157,760.
(60) Provisional application No. 62/677,658, filed on May 29, 2018.
(60) Provisional application No. 62/675,726, filed on May 23, 2018.

(51) Int. Cl. C07K 14/47; C12N 9/22; C12N 9/78

(52) U.S. Cl. CPC C07K 14/4702; C12N 9/22; C12N 9/78; C07K 2319/09; C12Y 305/04004; C12Y 305/04005

(57) ABSTRACT

Some aspects of this disclosure provide strategies, systems, reagents, methods, and kits that are useful for the targeted editing of nucleic acids, including editing a single site within the genome of a cell or subject, e.g., within the human genome. The disclosure provides fusion proteins of nucleic acid programmable DNA binding proteins (napDNAbp), e.g., Cas9 or variants thereof, and nucleic acid editing proteins such as cytidine deaminase domains (e.g., novel cytidine deaminases generated by ancestral sequence reconstruction), and adenosine deaminases that deaminate adenine in DNA. Aspects of the disclosure relate to fusion proteins (e.g., base editors) that have improved expression and/or localize efficiently to the nucleus. In some embodiments, base editors are codon optimized for expression in mammalian cells. In some embodiments, base editors include multiple nuclear localization sequences (e.g., bipartite NLSs), e.g., at least two NLSs. In some embodiments, methods for targeted nucleic acid editing are provided.

### FEDERALLY SPONSORED RESEARCH

This invention was made with government support under Grant No. HR0011-17-2-0049 awarded by the Department of Defense, and Grant Nos. HG009490, EB022376, GM118062, CA014051, and GM095450 awarded by the National Institutes of Health. The government has certain rights in the invention.

### BACKGROUND OF THE INVENTION

Targeted editing of nucleic acid sequences, for example, the targeted cleavage or the targeted introduction of a specific modification into genomic DNA, is a highly promising approach for the study of gene function and also has the potential to provide new therapies for human genetic diseases, for example, those caused by point mutations. Point mutations represent the majority of known human genetic variants associated with disease (1). Developing robust methods to introduce and correct point mutations is therefore an important challenge to understand and treat diseases with a genetic component.

Engineered base editors have been recently developed (2, 3). Base editors are fusions of catalytically disabled Cas moiety and a nucleobase modification enzyme (e.g., natural or evolved nucleobase deaminases). In some cases, base editors may also include proteins that alter cellular DNA repair processes to increase the efficiency and stability of the resulting single-nucleotide change, e.g., a UGI domain (2, 3).

Two classes of base editors have been generally described to date: cytidine base editors convert target C. G base pairs to T. A base pairs, and adenine base editors convert A. T base pairs to G. C base pairs. Collectively, these two classes of base editors enable the targeted installation of all four transition mutations (C-to-T, G-to-A, A-to-G, and T-to-C), which collectively account for about 61% of known human pathogenic small nucleotide polymorphisms (SNPs) in the ClinVar database. In addition, base editors have been used widely in organisms ranging from prokaryotes to plants to amphibians to mammals, and have even been used to correct pathogenic mutations in human embryos (4-18).

However, the utility of base editing is limited by several constraints, including the PAM requirement imposed by the particular Cas moiety used (e.g., naturally occurring Cas9 from S. pyogenes, or a modified version thereof, or a homolog thereof), off-target base editing of non-target nucleotides nearby the desired editing site, the production of undesired edited genomic byproducts (e.g., indels), and overall low editing efficiencies.

The development of “next-generation” base editors has begun to address some of these limitations, including base editors with different or expanded PAM compatibilities (19-21), highfidelity base editors with reduced off-target activity (20, 22-25), base editors with narrower editing windows (normally ˜5 nucleotides wide) (19), and a cytidine base editor (BE4) with reduced by-products (6).

Nevertheless, despite these recent advances, the efficiency of base editing by base editors varies widely by among other factors, cell type and target locus. Thus, there continues to be a significant need in the art for the development of base editors with improved editing efficiencies, and in particular, wherein the improvements are aimed to address those fundamental underlying biological aspects which restrict the genome editing efficiencies of base editor systems. The present disclosure provides improved base editors which overcome the problems in the art.

### SUMMARY OF THE INVENTION

The instant specification provides for improved base editors which overcome deficiencies of those in art. In particular, the specification provides base editors with improved editing efficiencies, for example, wherein the improvements address underlying biological aspects that limit the efficiency of genome editing achieved by existing base editor systems, including, for example, improved expression and/or nuclear localization. In addition, the instant specification provides for nucleic acid molecules encoding and/or expressing the improved base editors disclosed herein, as well as vectors for cloning and/or expressing the improved base editors described herein, host cells comprising said nucleic acid molecules and cloning and/or expression vectors, and compositions for delivering and/or administering nucleic acid-based embodiments described herein. In addition, the disclosure provides for improved base editors as described herein, as well as compositions comprising said improved base editors. Still further, the present disclosure provides for methods of making the base editors, as well as methods of using the improved base editors or nucleic acid molecules encoding the improved base editors in applications including editing a nucleic acid molecule, e.g., a genome, with improved efficiency as compared to base editor that forms the state of the art. The specification also provides methods for efficiently editing a target nucleic acid molecule, e.g., a single nucleobase of a genome, with a base editing system described herein (e.g., in the form of an improved base editor protein as described herein or a vector encoding same) and conducting based editing. Still further, the specification provides therapeutic methods for treating a genetic disease and/or for altering or changing a genetic trait or condition by contacting a target nucleic acid molecule, e.g., a genome, with a base editing system (e.g., in the form of an isolated improved base editor protein or a vector encoding same) and conducting base editing to treat the genetic disease and/or change the genetic trait (e.g., eye color).

The present inventors have surprisingly discovered various ways to improve the efficiency of base editing by recognizing that the fraction of cells expressing active base editors, and/or the amount of functional base editor protein produced by each cell, constitutes restrictions on the efficiency of base editing. In particular, the inventors have surprisingly discovered that by (a) improving nuclear localization of the expressed base editor or component thereof to the nucleus, (b) optimizing codon usage of the sequence encoding the base editor or component thereof, and (c) enhancing the expression of the sequence encoding the base editor or component thereof, or a combination thereof, e.g., by ancestral protein reconstruction (ASR), significantly improves the editing efficiencies of previously known base editors, e.g., cytidine base editors. Ancestral protein reconstruction uses an alignment of known protein sequences, an evolutionary model, and a resulting phylogenetic tree to infer ancestral protein sequences at the nodes of the phylogeny. See, Harms, M. J. et al., “Evolutionary biochemistry: revealing the historical and physical causes of protein properties.” Nature reviews. Genetics 14, 559-571 (2013); the entire contents of which are incorporated herein by reference. Indeed, ASR has been shown to improve the expression of a variety of proteins while retaining wild-type levels of biochemical activity. See, Wheeler, L. C., et al., “The thermostability and specificity of ancient proteins.” Curr Opin Struct Biol 38, 37-43 (2016); Nguyen, V. et al., “Evolutionary drivers of thermoadaptation in enzyme catalysis.” Science 355, 289-294 (2017); Wilson, C. et al. “Kinase dynamics. Using ancient protein kinases to unravel a modern cancer drug's mechanism.” Science 347, 882-886 (2015); and Risso, V. A., et al., “Hyperstability and substrate promiscuity in laboratory resurrections of Precambrian beta-lactamases.” J Am Chem Soc 135, 2899-2902 (2013); the entire contents of each of which are incorporated herein by reference.

These methods can be used to provide improved base editors that can be used to efficiently edit a nucleic acid molecule in a manner that is dramatically improved as compared to base editors known in the art. The improved base editors may be used to efficiently edit nucleic acid molecules, e.g., a genome, for example, by correcting a disease-causing point mutation.

Thus, in one aspect, the specification discloses a fusion protein comprising: (i) a nucleic acid programmable DNA binding protein (napDNAbp); (ii) a DNA effector domain; (iii) a first nuclear localization sequence; and (iv) a second nuclear localization sequence. In certain embodiments, the first nuclear localization sequence (NLS) and/or the second nuclear localization sequence is a bipartite nuclear localization sequence, for example a bipartite nuclear localization sequence that comprises the amino acid sequence of KRTADGSEFESPKKKRKV (SEQ ID NO: 1) or KRTADGSEFEPKKKRKV (SEQ ID NO: 2). Nuclear localization sequences may be at the N-terminus, and/or the C-terminus of the fusion proteins (e.g., base editors) provided herein. For example, any of the fusion proteins provided herein may have an N-terminal and a C-terminal NLS.

It should be appreciated that any of the fusion proteins provided herein contain a nucleic acid programmable DNA binding protein, such as a Cas9 domain, in order to bring the fusion protein in proximity to a target nucleic acid sequence (e.g., for the purposes of base editing). The nucleic acid programmable DNA binding protein may be a Cas9 domain, such as a Cas9 nickase domain. For example, the Cas9 nickase domain may be a Cas9 nickase that cuts a nucleic acid target strand of a nucleotide duplex, where the nucleotide target strand is the strand that binds a gRNA. As one example, the Cas9 domain comprises an amino acid sequence that is at least 80%, 85%, 90%, 95%, 98%, 99%, or 100% identical to the amino acid sequence of:

(SEQ ID NO: 3)
DKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFH
RLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFE
ENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKN
LSDAILLSDILRVNTEITKAPLSASMIKRYDEHHQDLTLLKALVRQQLP
EKYKEIFFDQSKNGYAGYIDGGASQEEFYKFIKPILEKMDGTEELLVKL
NREDLLRKQRTFDNGSIPHQIHLGELHAILRRQEDFYPFLKDNREKIEK
ILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKT
YAHLFDDKVMKQLKRRRYTGWGRLSRKLINGIRDKQSGKTILDFLKSDG
FANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIE
EGIKELGSQILKEHPVENTQLQNEKLYLYYLQNGRDMYVDQELDINRLS
DYDVDHIVPQSFLKDDSIDNKVLTRSDKNRGKSDNVPSEEVVKKMKNYW
RQLLNAKLITQRKFDNLTKAERGGLSELDKAGFIKRQLVETRQITKHVA
QILDSRMNTKYDENDKLIREVKVITLKSKLVSDFRKDFQFYKVREINNY
HHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRD
FATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKN
PIDFLEAKGYKEVKKDLIIKLPKYSLFELENGRKRMLASAGELQKGNEL
ALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAF
KYFDTTIDRKRYTSTKEVLDATLIHQSITGLYETRIDLSQLGGD.

In some aspects, the fusion proteins provided herein include an effector domain that is capabable of making a modification to a nucleic acid (e.g., DNA). For example, the DNA effector domain may be a deaminase domain, such as a cytidine deaminase domain or an adenosine deaminase domain. In certain embodiments, the deaminase domain is a cytidine deaminase domain, such as an APOBEC or AID cytidine deaminase. For base editing proteins that are capable of deaminating a cytidine to a uridine, e.g., to induce a C to T mutation in a DNA molecule, the cytidine deaminase can be a deaminase from the apolipoprotein B mRNA-editing complex (APOBEC) family deaminase. For example, the cytidine deaminase may comprise an APOBEC cytidine deaminase having an amino acid sequence that is at least 80%, 85%, 90%, 95%, 98%, 99%, or 100% identical to the amino acid sequence of: SSETGPVAVDPTLRRRIEPHEFEVFFDPRELRKETCLLYEINWGGRHSIWRHTSQNTN KHVEVNFIEKFTTERYFCPNTRCSITWFLSWSPCGECSRAITEFLSRYPHVTLFIYIARL YHHADPRNRQGLRDLISSGVTIQIMTEQESGYCWRNFVNYSPSNEAHWPRYPHLWV RLYVLELYCIILGLPPCLNILRRKQPQLTFFTIALQSCHYQRLPPHILWATGLK (SEQ ID NO: 4). The cytidine deaminase may also be an ancestral cytidine deaminase, such as any of the Anc689, Anc687, Anc686, Anc655, or Anc733 ancestral cytidine deaminases provided herein (e.g., any one of SEQ ID NOs: 5-9)

The fusion proteins provided herein, e.g., those that comprise two or more NLSs, may further include one or more Uracil-DNA glycosylase inhibitor (UGI) domains, which are capable of inhibiting Uracil-DNA glycosylase, thereby improving base editing efficiency of C to T base editor proteins. As one example, any of the fusion proteins provided herein comprise an amino acid sequence that is at least 80%, 85%, 90%, 95%, 98%, 99%, or 100% identical to the amino acid sequence of

(SEQ ID NO: 108)
TNLSDIIEKETGKQLVIQESILMLPEEVEEVIGNKPESDILVHTAYDES
TDENVMLLTSDAPEYKPWALVIQDSNGENKIKML.

It should be appreciated that the fusion proteins provided herein may be arranged in any conifguation, for example, the fusion protein may have the structure: NH₂-[first nuclear localization sequence]-[cytidine deaminase domain]-[Cas9 domain]-[first UGI domain]-[second UGI domain]-[second nuclear localization sequence]-COOH, and each instance of “-” comprises an optional linker. Linker sequences that may be used to link certain domains of the fusion protein are provided herein and may be modified to enhance the properties of the fusion proteins herein, such as base editing efficiency or modulating a base editing window.

In some aspects, any of the fusion proteins provided herein have an effector domain that includes an adenosine deaminase. Such fusion proteins may be used as adenosine base editing proteins, e.g., for generating an A to G mutation in a DNA molecule. Accordingly, in certain embodiments, the effector domain comprises an adenosine deaminase, for example an adenosine deaminase that deaminates an adenine in DNA. Adenosine deaminases that deaminate adenine in DNA have been described previously, for example in PCT/US2017/045381 (published as WO 2018/027078).

In certain embodiments, the adenosine deaminase comprises an amino acid sequence that is at least 80%, 85%, 90%, 95%, 98%, or 99% identical to the amino acid sequence of SEQ ID NO: 15, and includes one or more substitutions that confers the ability of the adenosine deaminase to deaminate adenine in DNA. For example, In certain embodiments, said one or more substitutions comprise a group of substitutions selected from the groups of substitutions consisting of: (i) W23L, H36L, P48A, R51L, L84F, A106V, D108N, H123Y, A142N, S146C, D147Y, R152P, E155V, 1156F, and K157N; (ii) W23R, H36L, P48A, R51L, L84F, A106V, D108N, H123Y, S146C, D147Y, R152P, E155V, 1156F, and K157N; (iii) H36L, P48S, R51L, L84F, A106V, D108N, H123Y, A142N, S146C, D147Y, E155V, 1156F, and K157N; (iv) H36L, P48S, R51L, L84F, A106V, D108N, H123Y, S146C, D147Y, E155V, 1156F, and K157N; (v) H36L, R51L, L84F, A106V, D108N, H123Y, S146C, D147Y, E155V, 1156F, and K157N; (vi) L84F, A106V, D108N, H123Y, D147Y, E155V, and 1156F; (vii) A106V, D108N, D147Y, and E155V; (viii) A106V and D108N; and (ix) D108N; of the amino acid sequence of SEQ ID NO: 15. It should be appreciated the the fusion proteins comprising an adenosine deaminase may further comprise a second adenosine deaminase, e.g., a TadA adenosine deaminase as set forth in SEQ ID NO: 15. Without wishing to be bound by any particular theory, dimerization of adenosine deaminase domains may improve base editing efficiency of any of the fusion proteins provided herein. As one example, the fusion protein may comprises the structure: NH₂-[first nuclear localization sequence]-[first adenosine deaminase]-[second adenosine deaminase]-[Cas9 domain]-[second nuclear localization sequence]-COOH, and each instance of “-” comprises an optional linker.

Some aspects of the disclosure provide nucleic acid sequences, e.g., DNA sequences encoding any of the fusion proteins, fusion protein domains (e.g., effector domains, napDNAbps, UGI domains) or linkers provided herein. In some embodiments, the DNA sequences are sequence optimized for expression in one or more cell types. For example, the DNA sequences may be optimized for expression in a mammalian cell (e.g., a HEK 293T cell). It should be appreciated that optimizing the codon usage of base editor constructs can greatly improve base editing efficiency. The DNA sequences may be codon optimized for expressin in a mammalian cell using Integrated DNA Technologies (IDT), GeneArt, Coller, and GenScript. Preferably, DNA sequences are codon optimized for expressin in a mammalian cell usingGenScript. As one example

In one aspect, the specification discloses a complex comprising any one of the presently disclosed fusion proteins and an RNA bound to the napDNAbp. In certain embodiments, the RNA is a guide RNA (gRNA). In certain embodiments, the RNA is a single guide RNA (sgRNA). In certain embodiments, the RNA comprises a nucleic acid sequence that targets SCN9a, MPDU1, or HBG

In one aspect, the specification discloses a method comprising contacting a nucleic acid (e.g., double stranded DNA) molecule with any of the presently disclosed complexes. The DNA may include a target sequence associated with a disease or disorder that may be corrected by contacting the complex with the DNA. In certain embodiments, the target sequence comprises a point mutation associated with a disease or disorder. For example, the target sequence may have a T to C point mutation associated with a disease or disorder, where the deamination of the mutant C base results in a sequence that is not associated with a disease or disorder. In certain embodiments, the target sequence comprises a G to A point mutation associated with a disease or disorder, where the deamination of the mutant A base results in a sequence that is not associated with a disease or disorder. The methods provided herein can be performed in vitro, such as in cell culture, or in vivo, such as in a subject.

In certain embodiments, the subject has been diagnosed with a disease or disorder. In certain embodiments, the disease or disorder is selected from the group consisting of congenital disorder of glycosylation type If, familial erythromyalgia, paroxysomal extreme pain disorder, chronic insensitivity to pain, sickle cell anemia, and β-thalassemia. In certain embodiments, the disease or disorder is associated with a point mutation in a MDPUI gene, a SCN9a gene or an HBG1 and/or an HBG2 gene.

In one aspect, the specification discloses pharmaceutical compositions comprising any of the presently disclosed fusion proteins, complexes, nucleic acids, and/or vectors. In certain embodiments, the pharmaceutical composition further comprises a pharmaceutically acceptable excipient, a lipid (e.g., a cationic lipid), and/or a polymer.

In one asepect, the disclosure provides ancestral cytidine deaminases, such as Anc689, Anc687 Anc686, Anc655, and Anc733. In another aspect, the specification provides ancestral cytidine deaminases that comprise an amino acid sequence that is at least 80%, 85%, 90%, 95%, 98%, or 99% identical to the amino acid sequence of any one of SEQ ID NOs: 5-9. In other embodiments, the ancestral cytidine deaminases comprise an amino acid sequence set forth in any one of SEQ ID NOs: 5-9. The application also provides for DNA sequences that encode such ancestral cytidine deaminases.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

FIGS. 1A-1E. Probing the factors that limit base editing efficiency in human cells. (FIG. 1A), BE4 (left) and ABE (right) induce the deamination of target C or target A nucleotides. They also nick the non-edited strand to direct DNA repair processes to replace that strand using the deaminated C (uracil, U) or the deaminated A (inosine, I) as a template. The result is BE4-mediated conversion of a target C·G base pair to a T·A base pair, and ABE-mediated conversion of a target A·T base pair to a G·C base pair. (FIG. 1B) Base pair changes required to correct pathogenic SNPs in the Clin Var database. The “1” wedge (47%) require conversion of the type mediated by ABE, while the “2” wedge (14%) require conversion of the type mediated by BE4. (FIG. 1C) Three base editor and fluorescent protein construct pairs used to elucidate the relationship between base editor expression and editing efficiency in human cells. All samples were transfected with an mCherry expression plasmid as a transfection control. In addition, cells were transfected with a BE4 expression plasmid (“in trans”), with a plasmid co-expressing both BE4 and GFP on separate promoters (“in cis”), or with a plasmid expressing BE4-P2A-GFP, where P2A is a self-cleaving peptide that liberates free BE4 and free GFP during translation of a single mRNA (“P2A”). (FIG. 1D) Percent mCherry-positive or GFP-positive HEK293T cells 3 days after transfection of the construct pairs in (FIG. 1C). (FIG. 1E) Target C·G-to-T·A editing efficiency for unsorted HEK293T cells and sorted populations of HEK293T cells. The sorted in trans cells were mCherry-positive, while the sorted in cis and P2A cells were dual mCherry-positive and GFP-positive. Values and error bars in (FIG. 1D) and (FIG. 1E) represent the mean and standard deviation of three biological replicates 3 days after transfection.

FIGS. 2A-2E. Optimization of the BE4 C·G-to-T·A base editor by improving nuclear localization, improving codon usage, and performing ancestral protein reconstruction of cytidine deaminases. (FIG. 2A) BE4 architecture and effects of six NLS configurations on BE4 base editing efficiency at five endogenous genomic loci in HEK293T cells. (FIG. 2B) Effects of five codon usage methods on base editing efficiency of bis-bpNLS-BE4 at five endogenous genomic loci in HEK293T cells. Codon optimizations are as follows: IDT, Integrated DNA Technologies; J C, Jeff Coller; G A, GeneArt; G S, GenScript; IDT-GS, IDT APOBEC+GenScript Cas9 nickase. (FIG. 2C) Phylogenetic tree for ancestral APOBEC reconstruction. Numbered dots denote ancestral APOBEC sequences assayed for base editing activity in (FIG. 2D). (FIG. 2D) Base editing activity of bis-bpNLS-BE4 constructs with GenScript codon optimization using the ancestral APOBEC domains in (FIG. 2C) at five endogenous genomic loci in HEK293T cells. (FIG. 2E) Comparison of BE4, bis-bpNLS-BE4 with GenScript codons (BE4max), and bis-bpNLS-BE4 with ancestral Anc689 APOBEC and GenScript codons (AncBE4max) at three endogenous genomic loci in HEK293T cells across eight different plasmid doses. Values and error bars in (FIGS. 2A-2B) and (FIGS. 2D-2E) represent the mean and standard deviation of three biological replicates 3 days after transfection, except two replicates were used for wild-type rat APOBEC1 in (FIG. 2D).

FIGS. 3A-3B. Optimization of the ABE 7.10 A·T-to-G·C base editor by improving nuclear localization and improving codon usage. (FIG. 3A) ABE architecture, effects of NLS configuration (SV40 versus bis-bpNLS), and effects of codon usage (IDT versus GenScript) on ABE base editing efficiency at five endogenous genomic loci in HEK293T cells. BP: bis-bpNLS; GS: GenScript codon usage. (FIG. 3B) Comparison of previously reported ABE 7.10, bis-bpNLS-ABE with IDT codons, and bis-bpNLS-ABE with GenScript codons (ABEmax) at three endogenous genomic loci in HEK293T cells across eight different plasmid doses. Values and error bars represent the mean and standard deviation of three biological replicates 3 days after transfection, except two biological replicates were obtained for the 750 ng dose of bis-bpNLS ABE 7.10 at Site 5 and Site 13 in FIG. 3B.

FIGS. 4A-4D. Comparison of optimized AncBE4max, BE4max, and ABEmax base editors with previously reported BE4 and ABE 7.10 for the correction of pathogenic SNPs. (FIG. 4A) C·G to T·A editing outcomes for the correction of the Leu119Pro T>C mutation in MDPUI driving congenital disorder of glycosylation (CDG) type If by BE4, BE4max, or AncBE4max, in unsorted or sorted patient-derived fibroblasts. Among sorted cells, BE4 samples were sorted for mCherry-positive cells, while BE4max-P2A-GFP and AncBE4max-P2A-GFP samples were sorted for GFP-positive cells. All other C·G to T·A edits in the editing window are silent and are not shown. (FIG. 4B) C·G to T·A editing outcomes for editing the 3′ splice acceptor in intron 6 of SCN9a in mouse N2a cells, unsorted or sorted as described in (FIG. 4A). (FIG. 4C) A·T to G·C editing outcomes for the installation of activating mutations at protospacer positions A5 and A8 (−116 A to G and −113 A to G) in the Bellla binding sites of both HBG1 and HBG2 fetal hemoglobin promoters by ABE 7.10 or ABEmax in unsorted or sorted HEK293T cells. Among sorted cells, ABE samples were sorted for mCherry-positive cells, and ABEmax-P2A-GFP samples were sorted for GFP-positive cells. (FIG. 4D) A·T to G·C editing outcomes for the installation of a mutation at protospacer position A3 (HBG-175 T to C) thought to be the strongest SNP known to mediate the activation of fetal hemoglobin expression⁴⁸ by ABE 7.10 or ABEmax in HEK293T cells, unsorted or sorted as described in (FIG. 4C). Values and error bars represent the mean and standard deviation of three biological replicates 3 days after transfection, except two biological replicates were obtained for AncBE4max for the SCN9a target.

FIGS. 5A-5B. Unpaired two-sided t-test p-values for NLS and codon optimizations. (FIG. 5A) Unpaired two-sided t-test p-values for previously reported BE4 (C-terminal SV40 NLS, IDT codons) compared to bis-bpNLS BE4 (IDT codons) at all Cs within the activity window across five endogenous genomic loci in HEK293T cells transfected with 750 ng of base editor plasmid and 250 ng of sgRNA plasmid. (FIG. 5B) Unpaired two-sided t-test p-values for BE4 bis-bpNLS using IDE codons compared to GenScript codons at all Cs within the activity window across five endogenous genomic loci in HEK293T cells transfected with 750 ng of base editor plasmid and 250 ng of sgRNA plasmid. * p≤0.05; ** p≤0.01.

FIGS. 6A-6B. C·G-to-T·A base editing outcomes for BE4 variants with chimeric codon usages. (FIG. 6A) C·G-to-T·A base editing outcomes of bis-bpNLS BE4 variants using full-length GenScript codon optimization (BE4max) compared to chimeric constructs in which the APOBEC1 and Cas9 nickase components are constructed with different codon usages in HEK293T cells. Chimeras include (APOBEC1-Cas9 nickase): GenScript-IDT, GenScript-Jin Soo Kim, IDT-Jin Soo Kim. (FIG. 6B) Comparison of BE4max and bis-bpNLS BE4 with chimeric IDT-GenScript codon usage at three endogenous genomic loci in HEK293T cells across eight different plasmid doses. Values and error bars represent the mean and standard deviation of three biological replicates 3 days after transfection.

FIG. 7. Multiple sequence alignment of rat APOBEC1 and reconstructed ancestral cytidine deaminases. Residues are shaded based on the degree of conservation. The sequences from top to bottom correspond to SEQ ID NOs: 4-9.

FIGS. 8A-8C. Unpaired two-sided t-test p-values for BE4, BE4max, and AncBE4max editing at three genomic loci. (FIG. 8A) Unpaired two-sided t-test p-values for BE4 compared to BE4max at HEK3 (C5), RNF2 (C6), and EMX1 (C4) across eight different base editor plasmid doses in HEK293T cells. (FIG. 8B) Unpaired two-sided t-test p-values for BE4 compared to AncBE4max at HEK3 (C5), RNF2 (C6), and EMX1 (C4) across eight different base editor plasmid doses in HEK293T cells. (FIG. 8C) Unpaired two-sided t-test p-values for BE4max compared to AncBE4max at HEK3 (C5), RNF2 (C6), and EMX1 (C4) across eight different base editor plasmid doses in HEK293T cells. * p≤0.05; ** p≤0.01; ** p≤0.001; **** p≤0.0001.

FIG. 9. Indel frequencies for BE4, BE4max, and AncBE4 at three genomic loci. Indel frequencies are shown for BE4, BE4max, and AncBE4max at HEK3, RNF2, and EMX1 sites across eight different plasmid doses in HEK293T cells. % Indel and Stdev values represent the mean and standard deviation of three biological replicates 3 days after transfection.

FIGS. 10A-10D. Improved mRNA levels, protein levels, and base editing from BE4max and AncBE4max compared with BE4. (FIG. 10A) BE4max and AncBE4max result in 3.7- and 5.2-fold higher mRNA levels, respectively, than BE4 in HEK293T cells 3 days after base editor and guide RNA plasmid transfection as determined by qRT-PCR. Base editor mRNA levels were normalized to β-actin levels by ΔΔCt. Normalized values were adjusted for transfection efficiency as determined by qPCR amplification of the bGH terminator sequence present on BE4 plasmids. (FIG. 10B) Western blot of C-terminal HA-tagged BE4, BE4max, and AncBE4max in HEK293T cells 3 days after plasmid transfection, visualizing with anti-HA (top) or anti-actin (bottom) antibodies. The mock sample is cells transfected with guide RNA plasmid alone. (FIG. 10C) BE4max-P2A-GFP and AncBE4max-P2A-GFP show higher GFP and mCherry double-positive cell populations compared to BE4-P2A-GFP for three genomic loci 3 days after transfection. (FIG. 10D) C·G-to-T·A base editing outcomes for GFP and mCherry dual-positive cells expressing BE4 P2A-GFP, BE4max-P2A-GFP, and AncBE4max-P2A-GFP at three genomic loci tested. Values and error bars in (FIG. 10A), (FIG. 10C), and (FIG. 10D) represent the mean and standard deviation of three biological replicates 3 days after transfection.

FIGS. 11A-11B. Unpaired two-sided t-test p-values editing by ABE and ABEmax. (FIG. 11A) Unpaired two-sided t-test p-values comparing ABE and ABEmax at five genomic loci tested with 750 ng of ABE editor and 250 ng gRNA in HEK293T cells. (FIG. 11B) Unpaired two-sided t-test p-values for ABE and ABEmax at Site 5 (A7), Site 13 (A5), and Site 16 (A5) across eight different plasmid doses in HEK293T cells. * p≤0.05; ** p<0.01; *** p<0.001; **** p≤0.0001.

FIG. 12. Indel frequencies for ABE versus ABEmax at three genomic loci. Indel frequencies are shown for ABE and ABEmax at Site 5, Site 13, and Site 16 across eight different plasmid doses in HEK293T cells. % Indel and Stdev values represent the mean and standard deviation of three biological replicates 3 days after transfection.

FIG. 13. C·G-to-T·A base editing of the 3′ splice acceptor of SCN9a intron 6 in sorted N2a cells. N2a cells were nucleofected with plasmids encoding AncBE4max-P2A-GFP and the targeting sgRNA. Following a 3 day incubation, GFP-positive cells were isolated by FACS and analyzed by HTS. The protospacer of the non-transcribed strand is shown here; C7 corresponds to +1 G and C8 corresponds to −1 G of the 3′ splice acceptor. The PAM is shown as the last GGG in the sequence. The sequence corresponds to SEQ ID NO: 71.

FIGS. 14A-14C. Nucleofection and transfection efficiencies in type If fibroblasts, N2a cells, and HEK293T cells for disease-associated targets. (FIG. 14A) Congenital disorder of glycosylation type If fibroblasts were nucleofected with a plasmid encoding mCherry, a plasmid encoding the targeting sgRNA, and a plasmid encoding BE4, BE4max-P2A-GFP, or AncBE4max-P2A-GFP, then sorted after 3 days. (FIG. 14B) N2a cells were transfected with a plasmid encoding mCherry, a plasmid encoding the targeting sgRNA, and a plasmid encoding BE4, BE4max-P2A-GFP, or AncBE4max-P2A-GFP, then sorted after 3 days. (FIG. 14C) HEK293T cells were transfected with a plasmid encoding mCherry, a plasmid encoding the targeting sgRNA, and a plasmid encoding ABE or ABEmax-P2A-GFP, then sorted after 3 days. Values and error bars represent the mean and standard deviation of three biological replicates 3 days after transfection, except two biological replicates were used for AncBE4max-P2A-GFP in N2a cells.

FIG. 15. 468-taxa unrooted phylogeny of APOBEC homologs. The tree is shaded according to the UNIPROT annotation of sequences within each clade. Dotted lines indicate sequences used as an outgroup to root the tree in FIG. 2C.

FIG. 16. Flow Sorting HEK293T negative control from FIGS. 1A-1E.

FIG. 17. Flow Sorting HEK293T GFP+control from FIGS. 1A-1E.

FIG. 18. Flow Sorting HEK293T mCherry+control from FIGS. 1A-1E.

FIG. 19. Flow Sorting Example HEK293T In trans sort from FIGS. 1A-1E.

FIG. 20 Flow Sorting Example HEK293T In cis sort from FIGS. 1A-1E.

FIG. 21. Flow Sorting Example HEK293T P2A sort (BE4-P2A-GFP) from FIGS. 1A-1E.

FIG. 22. Flow Sorting Example HEK293T P2A sort (BE4max-P2A-GFP) from FIGS. 1A-1E.

FIG. 23. Flow Sorting Example HEK293T P2A sort (AncBE4max-P2A-GFP) from FIGS. 1A-1E.

FIG. 24. Flow Sorting N2A negative control, SCN9a sites 1 and 2, from FIGS. 4A-4D.

FIG. 25. Flow Sorting N2A GFP+control, SCN9a sites 1 and 2, from FIGS. 4A-4D.

FIG. 26. Flow Sorting N2A mCherry+control, SCN9a sites 1 and 2, from FIGS. 4A-4D.

FIG. 27. Flow Sorting Example N2A BE4 mCherry In trans sort, SCN9a sites 1 and 2, from FIGS. 4A-4D.

FIG. 28. Flow Sorting Example N2A P2A sort (BE4max-P2A-GFP), SCN9a sites 1 and 2, from FIGS. 4A-4D.

FIG. 29. Flow Sorting Example N2A P2A sort (AncBE4max-P2A-GFP), SCN9a sites 1 and 2, from FIGS. 4A-4D.

FIG. 30. Flow Sorting CGD Type 1F Fibroblast negative control from FIGS. 4A-4D.

FIG. 31. Flow Sorting CGD Type 1F Fibroblast GFP+control from FIGS. 4A-4D.

FIG. 32. Flow Sorting CGD Type 1F Fibroblast mCherry+control from FIGS. 4A-4D.

FIG. 33. Flow Sorting CGD Type 1F Example Fibroblast In trans sort (BE4) from FIGS. 4A-4D.

FIG. 34. Flow Sorting CGD Type 1F Example Fibroblast P2A sort (BE4max-P2A-GFP) from FIG. 4.

FIG. 35. Flow Sorting CGD Type 1F Example Fibroblast P2A sort (AncBE4max-P2A-GFP) from FIGS. 4A-4D.

### DEFINITIONS

As used herein and in the claims, the singular forms “a,” “an,” and “the” include the singular and the plural reference unless the context clearly indicates otherwise. Thus, for example, a reference to “an agent” includes a single agent and a plurality of such agents.

### Adenosine Deaminase

As used herein, an “adenosine deaminase” is an enzyme that catalyzes the deamination of adenosine, converting it to the nucleoside hypoxanthine. Under standard Watson-Crick hydrogen bond pairing, an adenosine base hydrogen bonds to a thymine base (or a uracil in case of RNA). When adenine is converted to hypoxanthine, the hypoxanthine undergoes hydrogen bond pairing with cytosine. Thus, a conversion of “A” to hypoxanthine by adenosine deaminase will cause the insertion of “C” instead of a “T” during cellular repair and/or replication processes. Since the cytosine “C” pairs with guanine “G”, the adenosine deaminase in coordination with DNA replication causes the conversion of an A·T pairing to a C·G pairing in the double-stranded DNA molecule.

### Ancestral Sequence Reconstruction (ASR)

Ancestral sequence reconstruction (ASR) is the process of analyzing modern sequences within an evolutionary/phylogenetic context to infer the ancestral sequences at particular nodes of a tree using an ASR algorithm. ASR algorithms are known in the art.

### Base Editing

Base editing is a genome editing technology that involves the conversion of a specific nucleic acid base into another at a targeted genomic locus. In certain aspects, this can be achieved without requiring double-stranded DNA breaks (DSB). Since many genetic diseases arise from point mutations, this technology has important implications in the study of human health and disease.

To date, other genome editing techniques, including CRISPR-based systems, begin with the introduction of a DSB at a locus of interest. Subsequently, cellular DNA repair enzymes mend the break, commonly resulting in random insertions or deletions (indels) of bases at the site of the DSB. However, when the introduction or correction of a point mutation at a target locus is desired rather than stochastic disruption of the entire gene, these genome editing techniques are unsuitable, as correction rates are low (e.g., typically 0.1% to 5%), with the major genome editing products being indels. In order to increase the efficiency of gene correction without simultaneously introducing random indels, the present inventors previously modified the CRISPR/Cas9 system to directly convert one DNA base into another without DSB formation.

### Base Editors

The term “base editors (BEs)” or “nucleobase editors (NBEs)” or as used herein, refers to the improved Cas-fusion proteins described herein. In some embodiments, the fusion protein comprises a nuclease-inactive Cas9 (dCas9) fused to a deaminase which still binds DNA in a guide RNA-programmed manner via the formation of an R-loop, but does not cleave the DNA backbone. For example, the dCas9 of the fusion protein can comprise a D10A and a H840A mutation (which renders Cas9 capable of cleaving only one strand of a nucleic acid duplex) as described in PCT/US2016/058344 (published as WO 2017/070632), which is incorporated herein by reference in its entirety. In some embodiments, the fusion protein comprises a Cas9 nickase fused to a deaminase, e.g., a cytidine deaminase (rAPOBEC1) which converts a DNA base cytosine to uracil. One such base editor is referred to as “BE1” in the literature. In some embodiments, the fusion protein comprises a nuclease-inactive Cas9 fused to a deaminase and further fused to a UGI domain (uracil DNA glycosylase inhibitor, which prevents the subsequent U: G mismatch from being repaired back to a C: G base pair). One such base editor is referred to as “BE2” in the literature. In other embodiments, to improve base editing efficiency, the catalytic His residue at position 840 in the Cas9 HNH domain of BE2 can be restore (resulting in “BE3” as described in the literature), which nicks only the non-edited strand, simulating newly synthesized DNA and leading to the desired U: A product. In other embodiments, the dCas9 is any dCas9 disclosed or described in PCT/US2017/045381 (published as WO 2018/027078), which is incorporated herein by reference in its entirety. The terms “nucleobase editors (NBEs)” and “base editors (BEs)” may be used interchangeably. The term “base editors” encompasses any base editor known or described in the art at the time of this filing, but also the improved base editors described herein. The base editors known in the state of the art which may be modified by the methods and strategies described herein to improve editing efficiency include, for example, BE1, BE2, BE3, or BE4.

### Cas9 or Cas9 Moiety

The term “Cas9” or “Cas9 nuclease” or “Cas9 moiety” refers to a CRISPR associated protein 9, or functional fragment thereof, and embraces any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. More broadly, a Cas9 is a type of “RNA-programmable nuclease” or “RNA-guided nuclease” or more broadly a type of “nucleic acid programmable DNA binding protein (napDNAbp)”. The term Cas9 is not meant to be particularly limiting and may be referred to as a “Cas9 or equivalent.” Exemplary Cas9 proteins are further described herein and/or are described in the art and are incorporated herein by reference. The present disclosure is unlimited with regard to the particular Cas9 that is employed in the improved base editors of the invention.

dCas9

As used herein, the term “dCas9” refers to a nuclease-inactive Cas9 or nuclease-dead Cas9, or a functional fragment thereof, and embraces any naturally occurring dCas9 from any organism, any naturally-occurring dCas9 equivalent or functional fragment thereof, any dCas9 homolog, ortholog, or paralog from any organism, and any mutant or variant of a dCas9, naturally-occurring or engineered. The term dCas9 is not meant to be particularly limiting and may be referred to as a “dCas9 or equivalent.” Exemplary dCas9 proteins and method for making dCas9 proteins are further described herein and/or are described in the art and are incorporated herein by reference.

### Cytidine Deaminase

As used herein, a “cytidine deaminase” encoded by the CDA gene is an enzyme that catalyzes the removal of an amine group from cytidine (i.e., the base cytosine when attached to a ribose ring) to uridine (C to U) and deoxycytidine to deoxyuridine (C to U). A non-limiting example of a cytidine deaminase is APOBEC1. Under standard Watson-Crick hydrogen bond pairing, a cytosine base hydrogen bonds to a guanine base. When cytidine is converted to uridine (or deoxycytidine is converted to deoxyuridine), the uridine (or the uracil base of uridine) undergoes hydrogen bond pairing with the base adenine. Thus, a conversion of “C” to uridine (“U”) by cytidine deaminase will cause the insertion of “A” instead of a “G” during cellular repair and/or replication processes. Since the adenine “A” pairs with thymine “T”, the cytidine deaminase in coordination with DNA replication causes the conversion of an C·G pairing to a T. A pairing in the double-stranded DNA molecule.

### CRISPR

CRISPR is a family of DNA sequences (i.e., CRISPR clusters) in bacteria and archaca that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species—the guide RNA. Sec, e.g., Jinck M., Chylinski K., Fonfara I., Hauer M., Doudna J.A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an MI strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roc B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

### Deaminase

As used herein, the term “deaminase” or “deaminase domain” or “deaminase moiety” refers to a protein or enzyme that catalyzes a deamination reaction. In some embodiments, the deaminase is an adenosine deaminase, which catalyzes the hydrolytic deamination of adenine or adenosine (e.g., an engineered adenosine deaminase that deaminates adenosine in DNA). In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism. The term deaminase also embraces any genetically engineered deaminase that may comprise genetic modifications (e.g., one or more mutations) that results in a variant deaminase having an amino acid sequence comprising one or more changes relative to a wildtype counterpart deaminase. Examples of deaminases are given herein, and the term is not meant to be limiting.

### Effective Amount

The term “effective amount,” as used herein, refers to an amount of a biologically active agent that is sufficient to elicit a desired biological response. For example, in some embodiments, an effective amount of a base editor may refer to the amount of the base editor that is sufficient to edit a target site nucleotide sequence, e.g., a genome. In some embodiments, an effective amount of a base editor provided herein, e.g., of a fusion protein comprising a nuclease-inactive Cas9 domain and a nucleic acid editing domain (e.g., a deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a nuclease, a deaminase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

### Inhibitor of Base Repair

The term “inhibitor of base repair” or “IBR” refers to a protein that is capable of inhibiting the activity of a nucleic acid repair enzyme, for example a base excision repair enzyme. In some embodiments, the IBR is an inhibitor of inosine base excision repair. Exemplary inhibitors of base repair include inhibitors of APE1, Endo III, Endo IV, Endo V, Endo VIII, Fpg, hOGG1, hNEIL1, T7 EndoI, T4PDG, UDG, hSMUG1, and hAAG. In some embodiments, the IBR is an inhibitor of Endo V or hAAG. In some embodiments, the IBR is a catalytically inactive EndoV or a catalytically inactive hAAG.

### Isolated

As used herein, the term “isolated protein” or “isolated nucleic acid” refers to a protein or nucleic acid that by virtue of its origin or source of derivation is not associated with naturally associated components that accompany it in its native state; is substantially free of other proteins or nucleic acids from the same species; is expressed by a cell from a different species; or does not occur in nature. Thus, a polypeptide or nucleic acid that is chemically synthesized or synthesized in a cellular system different from the cell from which it naturally originates will be “isolated” from its naturally associated components. A protein or nucleic acid may also be rendered substantially free of naturally associated components by isolation, using protein purification techniques well known in the art.

### Linker

The term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a recombinase. In some embodiments, a linker joins a dCas9 and base editor moiety (e.g., a cytidine or adenosine deaminase). Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

### Mutation

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans, including Marfan syndrome which results from a mutation in the gene for the connective tissue protein called fibrillin. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively the mutation could lead to overexpression of one or more genes involved in control of the cell cycle, thus leading to uncontrolled cell division and hence to cancer. Because of their nature, gain-of-function mutations are usually dominant.

### Non-Naturally Occurring or Engineered

The terms “non-naturally occurring” or “engineered” are used interchangeably and indicate the involvement of the hand of man. The terms, when referring to nucleic acid molecules or polypeptides (e.g., Cas9 or deaminases) mean that the nucleic acid molecule or the polypeptide is at least substantially free from at least one other component with which they are naturally associated in nature and/or as found in nature (e.g., an amino acid sequence not found in nature).

### Nucleic Acid/Nucleic Acid Molecule

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues.

Nucleic Acid Programmable R/DNA Binding Protein (napR/DNAbp)

The term “nucleic acid programmable D/RNA binding protein (napR/DNAbp)” refers to any protein that may associate (e.g., form a complex) with one or more nucleic acid molecules (i.e., which may broadly be referred to as a “napR/DNAbp-programming nucleic acid molecule” and includes, for example, guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napR/DNAbp embraces CRISPR Cas 9 proteins, as well as Cas9 equivalents, homologs, orthologs, or paralogs, whether naturally occurring or non-naturally occurring (e.g., engineered or recombinant), and may include a Cas9 equivalent from any type of CRISPR system (e.g., type II, V, VI), including Cpf1 (a type-V CRISPR-Cas systems), C2c1 (a type V CRISPR-Cas system), C2c2 (a type VI CRISPR-Cas system) and C2c3 (a type V CRISPR-Cas system). Further Cas-equivalents are described in Makarova et al., “C2c2 is a single-component programmable RNA-guided RNA-targeting CRISPR effector,” Science 2016; 353 (6299), the contents of which are incorporated herein by reference. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao F, Shen XZ, Jiang F, Wu Y, Han C. DNA-guided genome editing using the Natronobacterium gregoryi Argonaute. Nat Biotechnol 2016; 34 (7): 768-73, which is incorporated herein by reference.

napR/DNAbp-programming nucleic acid molecule or guide sequence

The term “napR/DNAbp-programming nucleic acid molecule” or equivalently “guide sequence” refers the one or more nucleic acid molecules which associate with and direct or otherwise program a napR/DNAbp protein to localize to a specific target nucleotide sequence (e.g., a gene locus of a genome) that is complementary to the one or more nucleic acid molecules (or a portion or region thereof) associated with the protein, thereby causing the napR/DNAbp protein to bind to the nucleotide sequence at the specific target site. A non-limiting example is a guide RNA of a Cas protein of a CRISPR-Cas genome editing system.

### Nuclear Localization Signal (NLS)

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

### Nucleobase Modification Moiety or Nucleic Acid Effector Domain

The term, as used herein, “nucleobase modification moiety” or equivalently a “nucleic acid effector domain” embraces any protein, enzyme, or polypeptide (or functional fragment thereof) which is capable of modifying a DNA or RNA molecule. Nucleobase modification moieties can be naturally occurring, or can be recombinant. For example, a nucleobase modification moiety can include one or more DNA repair enzymes, for example, and an enzyme or protein involved in base excision repair (BER), nucleotide excision repair (NER), homology-dependnent recombinational repair (HR), non-homologous end-joining repair (NHEJ), microhomology end-joining repair (MMEJ), mismatch repair (MMR), direct reversal repair, or other known DNA repair pathway. A nucleobase modification moiety can have one or more types of enzymatic activities, including, but not limited to endonuclease activity, polymerase activity, ligase activity, replication activity, proofreading activity. Nucleobase modification moieties can also include DNA or RNA-modifying enzymes and/or mutagenic enzymes, such as, DNA methylases and deaminating enzymes (i.e., deaminases, including cytidine deaminases and adenosine deaminases, all defined above), which deaminate nucleobases leading in some cases to mutagenic corrections by way of normal cellular DNA repair and replication processes. The “nucleic acid effector domain” (e.g., a DNA effector domain or an RNA effector domain) as used herein may also refer to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments the nucleic acid editing domain is a deaminase (e.g., a cytidine deaminase, such as an APOBEC or an AID deaminase).

### Oligonucleotide/Polynucleotide

As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

### Protein/Peptide/Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a recombinase. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference. It should be appreciated that any of the disclosure provides any of the polypeptide sequences provided herein without an N-terminal methionine (M) residue.

### Recombinant

The term “recombinant” as used herein in the context of proteins or nucleic acids refers to proteins or nucleic acids that do not occur in nature, but are the product of human engineering. For exampe, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

### RNA-Programmable Nuclease/RNA-Guided Nuclease

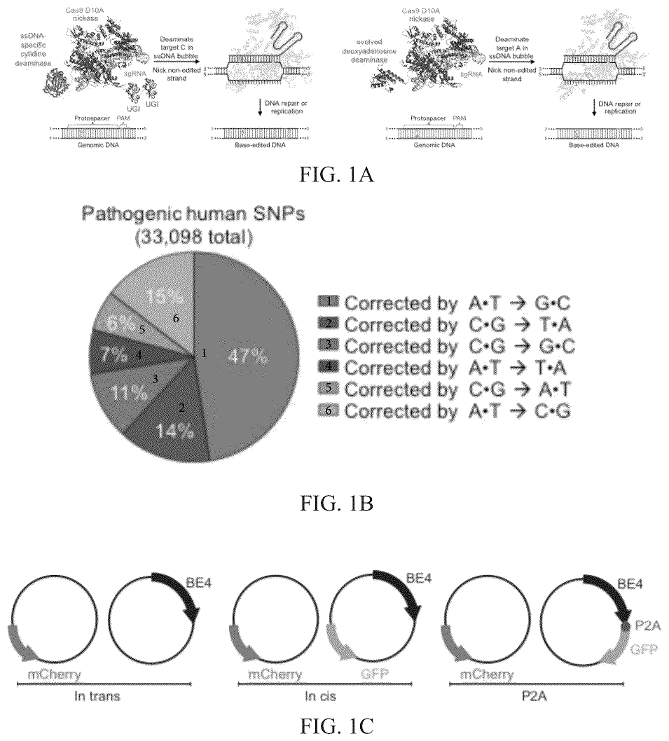

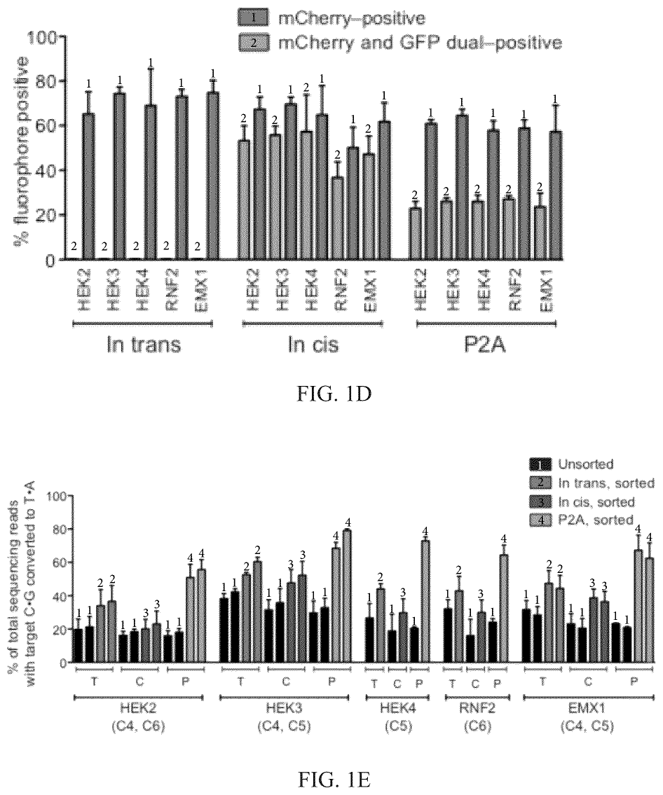

The term “RNA-programmable nuclease,” and “RNA-guided nuclease” are used interchangeably herein and refer to a nuclease that forms a complex with (e.g., binds or associates with) one or more RNA that is not a target for cleavage (e.g., a Cas9 or homolog or variant thereof). In some embodiments, an RNA-programmable nuclease, when in a complex with an RNA, may be referred to as a nuclease: RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821 (2012), the entire contents of which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof,” and U.S. Provisional Patent Application, U.S. Ser. No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease: RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an MI strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roc B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

Because RNA-programmable nucleases (e.g., Cas9) use RNA: DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development.

### Target Site

The term “target site” refers to a sequence within a nucleic acid molecule that is deaminated by a deaminase or a fusion protein comprising a deaminase (e.g., a dCas9-deaminase fusion protein provided herein).

### Uracil Glycosylase Inhibitor or UGI

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 10 or 108. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 10 or 108. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 10 or 108. In some embodiments, a UGI comprises an amino acid sequence homologous to the amino acid sequence set forth in SEQ ID NO: 10 or 108, or an amino acid sequence homologous to a fragment of the amino acid sequence set forth in SEQ ID NO: 10 or 108. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 10 or 108. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 10 or 108. In some embodiments, the UGI comprises the following amino acid sequence:

(SEQ ID NO: 10)
MTNLSDIIEKETGKQLVIQESILMLPEEVEEVIGNKPESDILVHTAYDE
STDENVMLLTSDAPEYKPWALVIQDSNGENKIKML
(P14739|UNGI_BPPB2 Uracil-DNA glycosylase
inhibitor).

### Treatment

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Variant

As used herein the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence.

### Wild Type

As used herein the term “wild type” is a term of the art understood by skilled persons and means the typical form of an organism, strain, gene or characteristic as it occurs in nature as distinguished from mutant or variant forms.

### Detailed Description of Certain Embodiments

The specification relates to improved base editors that achieve a significant increase in editing efficiencies by making various modifications which address certain underlying biological restrictions (e.g., restricted expression and nuclear localization) in known base editor systems surprisingly found to significantly reduce the efficiency of genome editing that is achievable by base editing. In particular, the inventors surprisingly found that base editor modifications (e.g., base editors comprising improved codon-usage and at least two NLSs) resulting in improved expression and nuclear localization and thereby improved editing efficiencies.

Despite these recent advances in the design of base editors, the efficiency of base editing varies widely. To increase base editing efficiency, the inventors sought to identify the factors that limit base editing efficiency in cells. It was surprisingly found by the inventors that expression and nuclear localization in human cells imposed key bottlenecks on editing efficiency. The inventors discovered that optimizing codon usage, using improved nuclear localization sequences (NLSs) (e.g., at least two NLS moieties), and performing ancestral reconstruction of deaminases resulted in base editors with greatly increased editing efficiency, often more than doubling target nucleotide conversion yields as compared to the unmodified counterpart editors. The resulting base editors were shown, as demonstrated in the Examples, to install point mutations relevant to human disease in a variety of mammalian cell types much more efficiently than previously described base editors. These approaches can be used to provide improved base editors that can be used to efficiently edit a nucleic acid molecule in a manner that is dramatically improved as compared to base editors known in the art. The improved base editors may be used to efficiently edit nucleic acid molecules, e.g., a genome, for example, by correcting a disease-causing point mutation.

Thus, the instant specification provides improved base editors that comprise one or modifications that result in increased expression (e.g., by way of optimizing codon usage and/or conducting ancestral reconstruction of deaminases) and/or nuclear localization. Further, the specification in certain aspects describes nucleic acid molecules encoding and/or expressing the improved base editors disclosed herein, as well as cloning and/or expression vectors for cloning and/or expressing the improved base editors described herein, host cells comprising said nucleic acid molecules and cloning and/or expression vectors, and compositions for delivering and/or administering nucleic acid-based embodiments described herein. In addition, the disclosure provides for improved base editors, as well as compositions comprising said improved base editors. Still further, the present disclosure provides for methods of making the improved base editors, as well as methods of using the improved base editors or nucleic acid molecules encoding the improved base editors in applications including editing a nucleic acid molecule, e.g., a genome, with improved efficiency as compared to base editor that forms the state of the art. The specification also provides methods for efficiently editing a target nucleic acid molecule, e.g., a single nucleobase of a genome, with a base editing system described herein (e.g., in the form of an improved base editor protein or a vector encoding same) and conducting based editing. Still further, the specification provides therapeutic methods for treating a genetic disease and/or for altering or changing a genetic trait or condition by contacting a target nucleic acid molecule, e.g., a genome, with a base editing system (e.g., in the form of an improved base editor protein or a vector encoding same) and conducting based editing to treat the genetic disease and/or change the genetic trait (e.g., eye color).

### I. Improved Base-Editors

In various aspects, the instant specification provides improved base editors that comprising one or modifications that result in increased expression (e.g., by way of optimizing codon usage and/or conducting ancestral reconstruction of deaminases) and/or nuclear localization (e.g., by incorporating at least two NLSs). The improved base editors described herein achieve a significant increase in editing efficiencies as compared to unmodified counterparts by making various modifications which address certain fundamental underlying biological restrictions in known base editor systems surprisingly found to significantly reduce the efficiency of genome editing that is achievable by base editing. In particular, the inventors surprising found that base editor modifications resulting in improved expression and nuclear localization specifically resulted in improved editing efficiencies.

In certain aspects, the methods described herein for modifying and improving base editors begins with a base editor known in the art upon which one or improvements are imparted. The state of the art has described numerous base editors as of this filing. The methods and approaches herein described for improving base editors may be applied to any previously known base editor, or to base editors that may be developed in the further but which lack the beneficial characteristics imparted by the instant methods and modification approaches. Examplary base editors that may be modified by the methods described herein to achieve the improved base editors of the invention can include, for example, those described in the following references and/or patent publications, each of which are incorporated by reference in their entireties: (a) PCT/US2014/070038 (published as WO2015/089406, Jun. 18, 2015) and its equivalents in the US or around the world; (b) PCT/US2016/058344 (published as WO2017/070632, Apr. 27, 2017) and its equivalents in the US or around the world; (c) PCT/US2016/058345 (published as WO2017/070633, April 27. 2017) and its equivalent in the US or around the world; (d) PCT/US2017/045381 (published as WO2018/027078, Feb. 8, 2018) and its equivalents in the US or around the world; (e) PCT/US2017/056671 (published as WO2018/071868, Apr. 19, 2018) and its equivalents in the US or around the world; PCT/2017/048390 (WO2017/048390, Mar. 23, 2017) and its equivalents in the US or around the world; (f) PCT/US2017/068114 (not published) and its equivalents in the US or around the world; (g) PCT/US2017/068105 (not published) and its equivalents in the US or around the world; (h) PCT/US2017/046144 (WO2018/031683, Feb. 15, 2018) and its equivalents in the US or around the world; (i) PCT/US2018/024208 (not published) and its equivalents in the US or around the world; (j) PCT/2018/021878 (WO2018/021878, Feb. 1, 2018) and its equivalents in the US and around the world; (k) Komor, A. C., Kim, Y. B., Packer, M. S., Zuris, J. A. & Liu, D. R. Programmable editing of a target base in genomic DNA without double-stranded DNA cleavage. Nature 533, 420-(2016); (1) Gaudelli, N. M. et al. Programmable base editing of A·T to G·C in genomic DNA without DNA cleavage. Nature 551, 464-(2017); (m) any of the references listed in this specification entitled “References” and which reports or describes a base editor known in the art.

In various aspects, the improved or modified base editors described herein have the following generalized structure:
A-B-C,
   wherein “A” is a Cas moiety or napDNAbp, “B” is nucleic acid effector domain (e.g., a deaminase, such as a cytidine or adenosine deaminase), and “C” is as least two nuclear localization signals (NLS). In addition, the “-” represents a linker that covalently joins moieties A, B, and C. The linkers can be any suitable type (e.g., amino acid sequences or other biopolymers, or synthetic chemical linkages in the case where the moieties are bioconjugated to one another) or length. In addition, a functional improved base editor of the invention would also include one or more “R” or guide sequences (e.g., guide RNA in the case of a Cas9 or Cas9 equivalent) in order to carry out the R/DNA-programmable functionality of base editors for targeting specific sites to be corrected.

The order of linkage of the moieties is not meant to be particularly limiting so long as the particular arrangement of the elements of moieties produces a functional base editor. That is, the improved base editors of the invention may also include editors represented by the following structures:
B-A-C;
B-C-A;
C-B-A;
C-A-B; and
A-C-B.

In some embodiments, the improved base editors provided herein can be made a recombinant fusion protein comprising one or more protein domains, thereby generating an improved base editor. In certain embodiments, the base editors provided herein comprise one or more features that improve the base editing activity (e.g., efficiency, selectivity, and/or specificity) of the base editor proteins. For example, the base editor proteins provided herein may comprise a Cas9 domain that has reduced nuclease activity. In some embodiments, the base editor proteins provided herein may have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand containing a T opposite the targeted A. Mutation of the catalytic residue (e.g., D10 to A10) of Cas9 prevents cleavage of the edited strand containing the targeted A residue. Such Cas9 variants are able to generate a single-strand DNA break (nick) at a specific location based on the gRNA-defined target sequence, leading to repair of the non-edited strand, ultimately resulting in a T to C change on the non-edited strand.

In some embodiments, any of the base editor proteins provided herein may further comprise one or more additional nucleic acid effector moieties, such as, for example, an inhibitor of inosine base excision repair (e.g., a uracil glycosylase inhibitor (UGI) domain or a catalytically inactive inosine-specific nuclease (dISN)). Without wishing to be bound by any particular theory, the UGI domain or dISN may inhibit or prevent base excision repair of a deaminated adenosine residue (e.g., inosine), which may improve the activity or efficiency of the base editor.

### The Cas9 Moiety or Equivalent Protein

The improved base editors provided by the instant specification include any suitable Cas9 moiety or equivalent protein, such as a CRISPR associated protein 9, or functional fragment thereof, and embraces any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. More broadly, a Cas9 is a type of “RNA-programmable nuclease” or “RNA-guided nuclease” or “nucleic acid programmable DNA-binding protein.” The terms napR/DNAbp or Cas9 are not meant to be particularly limiting. The present disclosure is unlimited with regard to the particular napR/DNAbp, Cas9 or Cas9 equivalent that is employed in the improved base editors of the invention.

In some embodiments, the napR/DNAbp is a Cas moiety.

In various embodiment, the Cas moiety is a S. pyogenes Cas9, which has been mostly widely used as a tool for genome engineering. This Cas9 protein is a large, multi-domain protein containing two distinct nuclease domains. Point mutations can be introduced into Cas9 to abolish nuclease activity, resulting in a dead Cas9 (dCas9) that still retains its ability to bind DNA in a sgRNA-programmed manner. In principle, when fused to another protein or domain, dCas9 can target that protein to virtually any DNA sequence simply by co-expression with an appropriate sgRNA.

In other embodiments, the Cas moiety is a Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); or Neisseria. meningitidis (NCBI Ref: YP_002342100.1).

In still other embodiments, the Cas moiety may include any CRISPR associated protein, including but not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Cse1, Cse2, Csa5, Csn2. Csm2, Csm3, Csm4, Csm5, Csm6, Cmr1, Cmr3, Cmr4, Cmr5, Cmr6, Csb1, Csb2, Csb3, Csx17, Csx14, Csx 10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A.

A Cas moiety may also be referred to as a casn1 nuclease or a CRISPR (clustered regularly interspaced short palindromic repeat)-associated nuclease. As outlined above, CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J.A., Charpentier E. Science 337:816-821 (2012), the entire contents of which is hereby incorporated by reference.

Cas9 and equivalents recognizes a short motif in the CRISPR repeat sequences (the PAM or protospacer adjacent motif) to help distinguish self versus non-self. As noted herein, Cas9 nuclease sequences and structures are well known to those of skill in the art (see, e.g., “Complete genome sequence of an MI strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference).

The Cas moiety may include any suitable homologs and/or orthologs. Cas9 homologs and/or orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.

In various embodiments, the improved base editors may comprise a nuclease-inactivated Cas protein may interchangeably be referred to as a “dCas” or “dCas9” protein (for nuclease-“dead” Cas9). Methods for generating a Cas9 protein (or a fragment thereof) having an inactive DNA cleavage domain are known (See, e.g., Jinck et al., Science. 337:816-821 (2012); Qi et al., “Repurposing CRISPR as an RNA-Guided Platform for Sequence-Specific Control of Gene Expression” (2013) Cell. 28; 152 (5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinck et al., Science. 337:816-821 (2012); Qi et al., Cell. 28;152 (5): 1173-83 (2013)). In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9.

In some embodiments, proteins comprising Cas9 or fragments thereof are referred to as “Cas9 variants.” A Cas9 variant shares homology to Cas9, or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length. In some embodiments, wild type Cas9 corresponds to Cas9 from Streptococcus pyogenes (NCBI Reference Sequence: NC_017053.1). In other embodiments, wild type Cas9 corresponds to Cas9 from Streptococcus pyogenes (NCBI Reference Sequence: NC_002737.2). In still other embodiments, dCas9 corresponds to, or comprises in part or in whole, a Cas9 amino acid sequence having one or more mutations that inactivate the Cas9 nuclease activity.

In some embodiments, the Cas9 domain comprises a D10A mutation, while the residue at position 840 relative to a wild type sequence such as Cas9 from Streptococcus pyogenes (NCBI Reference Sequence: NC_017053.1).

Without wishing to be bound by any particular theory, the presence of the catalytic residue H840 restores the activity of the Cas9 to cleave the non-edited (e.g., non-deaminated) strand containing a G opposite the targeted C. Restoration of H840 (e.g., from A840) does not result in the cleavage of the target strand containing the C. Such Cas9 variants are able to generate a single-strand DNA break (nick) at a specific location based on the gRNA-defined target sequence, leading to repair of the non-edited strand, ultimately resulting in a G to A change on the non-edited strand. Briefly, the C of a C-G basepair can be deaminated to a U by a deaminase, e.g., an APOBEC deaminase. Nicking the non-edited strand, having the G, facilitates removal of the G via mismatch repair mechanisms. UGI inhibits UDG, which prevents removal of the U.

In other embodiments, dCas9 variants having mutations other than D10A and H840A are provided, which, e.g., result in nuclease inactivated Cas9 (dCas9). Such mutations, by way of example, include other amino acid substitutions at D10 and H820, or other substitutions within the nuclease domains of Cas9 (e.g., substitutions in the HNH nuclease subdomain and/or the RuvC1 subdomain) with reference to a wild type sequence such as Cas9 from Streptococcus pyogenes (NCBI Reference Sequence: NC_017053.1). In some embodiments, variants or homologues of dCas9 (e.g., variants of Cas9 from Streptococcus pyogenes (NCBI Reference Sequence: NC_017053.1)) are provided which are at least about 70% identical, at least about 80% identical, at least about 90% identical, at least about 95% identical, at least about 98% identical, at least about 99% identical, at least about 99.5% identical, or at least about 99.9% identical to NCBI Reference Sequence: NC_017053.1. In some embodiments, variants of dCas9 (e.g., variants of NCBI Reference Sequence: NC_017053.1) are provided having amino acid sequences which are shorter, or longer than NC_017053.1 by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, the base editors as provided herein comprise the full-length amino acid sequence of a Cas9 protein, e.g., one of the Cas9 sequences provided herein. In other embodiments, however, fusion proteins as provided herein do not comprise a full-length Cas9 sequence, but only a fragment thereof. For example, in some embodiments, a Cas9 fusion protein provided herein comprises a Cas9 fragment, wherein the fragment binds crRNA and tracrRNA or sgRNA, but does not comprise a functional nuclease domain, e.g., in that it comprises only a truncated version of a nuclease domain or no nuclease domain at all. Exemplary amino acid sequences of suitable Cas9 domains and Cas9 fragments are provided herein, and additional suitable sequences of Cas9 domains and fragments will be apparent to those of skill in the art.

It should be appreciated that additional Cas9 proteins (e.g., a nuclease dead Cas9 (dCas9), a Cas9 nickase (nCas9), or a nuclease active Cas9), including variants and homologs thereof, are within the scope of this disclosure. Exemplary Cas9 proteins include, without limitation, those provided below. In some embodiments, the Cas9 protein is a nuclease dead Cas9 (dCas9). In some embodiments, the dCas9 comprises the amino acid sequence (SEQ ID NO: 53). In some embodiments, the Cas9 protein is a Cas9 nickase (nCas9). In some embodiments, the nCas9 comprises the amino acid sequence (SEQ ID NO: 54). In some embodiments, the Cas9 protein is a nuclease active Cas9. In some embodiments, the nuclease active Cas9 comprises the amino acid sequence (SEQ ID NO: 55).

Exemplary catalytically inactive Cas9 (dCas9)
(SEQ ID NO: 53):
(SEQ ID NO: 53)
DKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSF
FHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYN
QLFEENPINASGVDAKAILSARLSKSRRLENLIAQLPGEKKNGLFGNL
IALSLGLTPNFKSNFDLAEDAKLQLSKDTYDDDLDNLLAQIGDQYADL
FLAAKNLSDAILLSDILRVNTEITKAPLSASMIKRYDEHHQDLTLLKA
LVRQQLPEKYKEIFFDQSKNGYAGYIDGGASQEEFYKFIKPILEKMDG
TEELLVKLNREDLLRKQRTFDNGSIPHQIHLGELHAILRRQEDFYPFL
KDNREKIEKILTFRIPYYVGPLARGNSRFAWMTRKSEETITPWNFEEV
VDKGASAQSFIERMTNFDKNLPNEKVLPKHSLLYEYFTVYNELTKVKY
VTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTL
FEDREMIEERLKTYAHLFDDKVMKOLKRRRYTGWGRLSRKLINGIRDK
QSGKTILDFLKSDGFANRNFMQLIHDDSLTFKEDIQKAQVSGQGDSLH
EHIANLAGSPAIKKGILQTVKVVDELVKVMGRHKPENIVIEMARENQT
TQKGQKNSRERMKRIEEGIKELGSQILKEHPVENTQLQNEKLYLYYLQ
NGRDMYVDQELDINRLSDYDVDAIVPQSFLKDDSIDNKVLTRSDKNRG
KSDNVPSEEVVKKMKNYWRQLLNAKLITQRKFDNLTKAERGGLSELDK
AGFIKRQLVETRQITKHVAQILDSRMNTKYDENDKLIREVKVITLKSK
LVSDFRKDFQFYKVREINNYHHAHDAYLNAVVGTALIKKYPKLESEFV
YGDYKVYDVRKMIAKSEQEIGKATAKYFFYSNIMNFFKTEITLANGEI
RKRPLIETNGETGEIVWDKGRDFATVRKVLSMPQVNIVKKTEVQTGGF
SKESILPKRNSDKLIARKKDWDPKKYGGFDSPTVAYSVLVVAKVEKGK
SKKLKSVKELLGITIMERSSFEKNPIDFLEAKGYKEVKKDLIIKLPKY
SLFELENGRKRMLASAGELQKGNELALPSKYVNFLYLASHYEKLKGSP
EDNEQKQLFVEQHKHYLDEIIEQISEFSKRVILADANLDKVLSAYNKH
RDKPIREQAENIIHLFTLTNLGAPAAFKYFDTTIDRKRYTSTKEVLDA
TLIHQSITGLYETRIDLSQLGGD
Exemplary Cas9 nickase (nCas9) (SEQ ID NO: 54):
(SEQ ID NO: 54)
DKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSF
FHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYN
QLFEENPINASGVDAKAILSARLSKSRRLENLIAQLPGEKKNGLFGNL
IALSLGLTPNFKSNFDLAEDAKLQLSKDTYDDDLDNLLAQIGDQYADL
FLAAKNLSDAILLSDILRVNTEITKAPLSASMIKRYDEHHQDLTLLKA
LVRQQLPEKYKEIFFDQSKNGYAGYIDGGASQEEFYKFIKPILEKMDG
TEELLVKLNREDLLRKQRTFDNGSIPHQIHLGELHAILRRQEDFYPFL
KDNREKIEKILTFRIPYYVGPLARGNSRFAWMTRKSEETITPWNFEEV
VDKGASAQSFIERMTNFDKNLPNEKVLPKHSLLYEYFTVYNELTKVKY
VTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTL
FEDREMIEERLKTYAHLFDDKVMKQLKRRRYTGWGRLSRKLINGIRDK
QSGKTILDFLKSDGFANRNFMQLIHDDSLTFKEDIQKAQVSGQGDSLH
EHIANLAGSPAIKKGILQTVKVVDELVKVMGRHKPENIVIEMARENQT
TQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRG
KSDNVPSEEVVKKMKNYWRQLLNAKLITQRKFDNLTKAERGGLSELDK
AGFIKRQLVETRQITKHVAQILDSRMNTKYDENDKLIREVKVITLKSK
LVSDFRKDFQFYKVREINNYHHAHDAYLNAVVGTALIKKYPKLESEFV
YGDYKVYDVRKMIAKSEQEIGKATAKYFFYSNIMNFFKTEITLANGEI
RKRPLIETNGETGEIVWDKGRDFATVRKVLSMPQVNIVKKTEVQTGGF
SKESILPKRNSDKLIARKKDWDPKKYGGFDSPTVAYSVLVVAKVEKGK
SKKLKSVKELLGITIMERSSFEKNPIDFLEAKGYKEVKKDLIIKLPKY
SLFELENGRKRMLASAGELQKGNELALPSKYVNFLYLASHYEKLKGSP
EDNEQKQLFVEQHKHYLDEIIEQISEFSKRVILADANLDKVLSAYNKH
RDKPIREQAENIIHLFTLTNLGAPAAFKYFDTTIDRKRYTSTKEVLDA
TLIHQSITGLYETRIDLSQLGGD
Exemplary catalytically active Cas9
(SEQ ID NO: 55):
(SEQ ID NO: 55)
DKKYSIGLDIGTNSVGWAVITDEYKVPSKKFKVLGNTDRHSIKKNLIG
ALLFDSGETAEATRLKRTARRRYTRRKNRICYLQEIFSNEMAKVDDSF
FHRLEESFLVEEDKKHERHPIFGNIVDEVAYHEKYPTIYHLRKKLVDS
TDKADLRLIYLALAHMIKFRGHFLIEGDLNPDNSDVDKLFIQLVQTYN
QLFEENPINASGVDAKAILSARLSKSRRLENLIAQLPGEKKNGLFGNL
IALSLGLTPNFKSNFDLAEDAKLQLSKDTYDDDLDNLLAQIGDQYADL
FLAAKNLSDAILLSDILRVNTEITKAPLSASMIKRYDEHHQDLTLLKA
LVRQQLPEKYKEIFFDQSKNGYAGYIDGGASQEEFYKFIKPILEKMDG
TEELLVKLNREDLLRKQRTFDNGSIPHQIHLGELHAILRRQEDFYPFL
KDNREKIEKILTFRIPYYVGPLARGNSRFAWMTRKSEETITPWNFEEV
VDKGASAQSFIERMTNFDKNLPNEKVLPKHSLLYEYFTVYNELTKVKY
VTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTL
FEDREMIEERLKTYAHLFDDKVMKQLKRRRYTGWGRLSRKLINGIRDK
QSGKTILDFLKSDGFANRNFMQLIHDDSLTFKEDIQKAQVSGQGDSLH
EHIANLAGSPAIKKGILQTVKVVDELVKVMGRHKPENIVIEMARENQT
TQKGQKNSRERMKRIEEGIKELGSQILKEHPVENTQLQNEKLYLYYLQ
NGRDMYVDQELDINRLSDYDVDHIVPQSFLKDDSIDNKVLTRSDKNRG
KSDNVPSEEVVKKMKNYWRQLLNAKLITQRKFDNLTKAERGGLSELDK
AGFIKRQLVETRQITKHVAQILDSRMNTKYDENDKLIREVKVITLKSK
LVSDFRKDFQFYKVREINNYHHAHDAYLNAVVGTALIKKYPKLESEFV
YGDYKVYDVRKMIAKSEQEIGKATAKYFFYSNIMNFFKTEITLANGEI
RKRPLIETNGETGEIVWDKGRDFATVRKVLSMPQVNIVKKTEVQTGGF
SKESILPKRNSDKLIARKKDWDPKKYGGFDSPTVAYSVLVVAKVEKGK
SKKLKSVKELLGITIMERSSFEKNPIDFLEAKGYKEVKKDLIIKLPKY
SLFELENGRKRMLASAGELQKGNELALPSKYVNFLYLASHYEKLKGSP
EDNEQKQLFVEQHKHYLDEIIEQISEFSKRVILADANLDKVLSAYNKH
RDKPIREQAENIIHLFTLTNLGAPAAFKYFDTTIDRKRYTSTKEVLDA
TLIHQSITGLYETRIDLSQLGGD.

In some embodiments, a Cas moiety refers to a Cas9 or Cas9 homolog from archaea (e.g. nanoarchaea), which constitute a domain and kingdom of single-celled prokaryotic microbes. In some embodiments, Cas9 refers to CasX or CasY, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure.

In some embodiments, the Cas9 moiety is a nucleic acid programmable DNA binding protein (napDNAbp) of any of the fusion proteins provided herein may be a CasX or CasY protein. In some embodiments, the napDNAbp is a CasX protein. In some embodiments, the napDNAbp is a CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring CasX or CasY protein. In some embodiments, the napDNAbp is a naturally-occurring CasX or CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein. In some embodiments, the napDNAbp comprises an amino acid sequence of any one of SEQ ID NOs: 56-58. It should be appreciated that CasX and CasY from other bacterial species may also be used in accordance with the present disclosure. These sequences are shown below.

CasX (uniprot.org/uniprot/F0NN87;
uniprot.org/uniprot/F0NH53)
>tr|F0NN87|F0NN87_SULIH
CRISPR-associated Casx protein
OS = Sulfolobus islandicus (strain HVE10/4)
GN = SiH_0402 PE = 4 SV = 1
(SEQ ID NO: 56)
MEVPLYNIFGDNYIIQVATEAENSTIYNNKVEIDDEELRNVLNLAYKIAKN
NEDAAAERRGKAKKKKGEEGETTTSNIILPLSGNDKNPWTETLKCYNFPTT
VALSEVFKNFSQVKECEEVSAPSFVKPEFYEFGRSPGMVERTRRVKLEVEP
HYLIIAAAGWVLTRLGKAKVSEGDYVGVNVFTPTRGILYSLIQNVNGIVPG
IKPETAFGLWIARKVVSSVTNPNVSVVRIYTISDAVGQNPTTINGGFSIDL
TKLLEKRYLLSERLEAIARNALSISSNMRERYIVLANYIYEYLTGSKRLED
LLYFANRDLIMNLNSDDGKVRDLKLISAYVNGELIRGEG
CasX OS = Sulfolobus islandicus (strain REY15A)
>tr|F0NH53|F0NH53_SULIR CRISPR associated protein,
CasX OS = Sulfolobus islandicus (strain REY15A)
GN = SiRe_0771 PE = 4 SV = 1
(SEQ ID NO: 57)
MEVPLYNIFGDNYIIQVATEAENSTIYNNKVEIDDEELRNVLNLAYKIAKN
NEDAAAERRGKAKKKKGEEGETTTSNIILPLSGNDKNPWTETLKCYNFPTT
VALSEVFKNFSQVKECEEVSAPSFVKPEFYKFGRSPGMVERTRRVKLEVEP
HYLIMAAAGWVLTRLGKAKVSEGDYVGVNVFTPTRGILYSLIQNVNGIVPG
IKPETAFGLWIARKVVSSVTNPNVSVVSIYTISDAVGQNPTTINGGFSIDL
TKLLEKRDLLSERLEAIARNALSISSNMRERYIVLANYIYEYLTGSKRLED
LLYFANRDLIMNLNSDDGKVRDLKLISAYVNGELIRGEG
CasY (ncbi.nlm.nih.gov/protein/APG80656.1)
>APG80656.1 CRISPR-associated protein CasY
[uncultured Parcubacteria group bacterium]
(SEQ ID NO: 58)
MSKRHPRISGVKGYRLHAQRLEYTGKSGAMRTIKYPLYSSPSGGRTVPREI
VSAINDDYVGLYGLSNFDDLYNAEKRNEEKVYSVLDFWYDCVQYGAVESYT
APGLLKNVAEVRGGSYELTKTLKGSHLYDELQIDKVIKFLNKKEISRANGS
LDKLKKDIIDCFKAEYRERHKDQCNKLADDIKNAKKDAGASLGERQKKLFR
DFFGISEQSENDKPSFTNPLNLTCCLLPFDTVNNNRNRGEVLFNKLKEYAQ
KLDKNEGSLEMWEYIGIGNSGTAFSNFLGEGFLGRLRENKITELKKAMMDI
TDAWRGQEQEEELEKRLRILAALTIKLREPKFDNHWGGYRSDINGKLSSWL
QNYINQTVKIKEDLKGHKKDLKKAKEMINRFGESDTKEEAVVSSLLESIEK
IVPDDSADDEKPDIPAIAIYRRFLSDGRLTLNRFVQREDVQEALIKERLEA
EKKKKPKKRKKKSDAEDEKETIDFKELFPHLAKPLKLVPNFYGDSKRELYK
KYKNAAIYTDALWKAVEKIYKSAFSSSLKNSFFDTDFDKDFFIKRLQKIFS
VYRRFNTDKWKPIVKNSFAPYCDIVSLAENEVLYKPKQSRSRKSAAIDKNR
VRLPSTENIAKAGIALARELSVAGFDWKDLLKKEEHEEYIDLIELHKTALA
LLLAVTETQLDISALDFVENGTVKDFMKTRDGNLVLEGRFLEMFSQSIVFS
ELRGLAGLMSRKEFITRSAIQTMNGKQAELLYIPHEFQSAKITTPKEMSRA
FLDLAPAEFATSLEPESLSEKSLLKLKQMRYYPHYFGYELTRTGQGIDGGV
AENALRLEKSPVKKREIKCKQYKTLGRGQNKIVLYVRSSYYQTQFLEWFLH
RPKNVQTDVAVSGSFLIDEKKVKTRWNYDALTVALEPVSGSERVFVSQPFT
IFPEKSAEEEGQRYLGIDIGEYGIAYTALEITGDSAKILDQNFISDPQLKT
LREEVKGLKLDQRRGTFAMPSTKIARIRESLVHSLRNRIHHLALKHKAKIV
YELEVSRFEEGKQKIKKVYATLKKADVYSEIDADKNLQTTVWGKLAVASEI
SASYTSQFCGACKKLWRAEMQVDETITTQELIGTVRVIKGGTLIDAIKDFM
RPPIFDENDTPFPKYRDFCDKHHISKKMRGNSCLFICPFCRANADADIQAS
QTIALLRYVKEEKKVEDYFE

In various embodiments, the nucleic acid programmable DNA binding proteins include, without limitation, Cas9 (e.g., dCas9 and nCas9), CasX, CasY, Cpf1, C2c1, C2c2, C2C3, and Argonaute. One example of a nucleic acid programmable DNA-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

Also useful in the present compositions and methods are nuclease-inactive Cpf1 (dCpf1) variants that may be used as a guide nucleotide sequence-programmable DNA-binding protein domain. The Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 (SEQ ID NO: 65) inactivates Cpf1 nuclease activity. In some embodiments, the dCpf1 of the present disclosure comprises mutations corresponding to D917A, E1006A, D1255A, D917A/E1006A, D917A/D1255A, E1006A/D1255A, or D917A/E1006A/D1255A in SEQ ID NO: 59. It is to be understood that any mutations, e.g., substitution mutations, deletions, or insertions that inactivate the RuvC domain of Cpf1, may be used in accordance with the present disclosure.

In some embodiments, the nucleic acid programmable DNA binding protein (napDNAbp) of any of the fusion proteins provided herein may be a Cpf1 protein. In some embodiments, the Cpf1 protein is a Cpf1 nickase (nCpf1). In some embodiments, the Cpf1 protein is a nuclease inactive Cpf1 (dCpf1). In some embodiments, the Cpf1, the nCpf1, or the dCpf1 comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 59-66. In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 59-66, and comprises mutations corresponding to D917A, E1006A, D1255A, D917A/E1006A, D917A/D1255A, E1006A/D1255A, or D917A/E1006A/D1255A in SEQ ID NO: 59. In some embodiments, the dCpf1 comprises an amino acid sequence of any one SEQ ID NOs: 59-66. It should be appreciated that Cpf1 from other bacterial species may also be used in accordance with the present disclosure.

Wild type Francisella novicida Cpf1 (SEQ ID NO: 59)
(D917, E1006, and D1255 are bolded and underlined)
(SEQ ID NO: 59)
MSIYQEFVNKYSLSKTLRFELIPQGKTLENIKARGLILDDEKRAKDYKKAKQIIDKYHQFFIEEIL
SSVCISEDLLQNYSDVYFKLKKSDDDNLQKDFKSAKDTIKKQISEYIKDSEKFKNLFNQNLIDAKK
GQESDLILWLKQSKDNGIELFKANSDITDIDEALEIIKSFKGWTTYFKGFHENRKNVYSSNDIPTS
IIYRIVDDNLPKFLENKAKYESLKDKAPEAINYEQIKKDLAEELTFDIDYKTSEVNQRVFSLDEVF
EIANFNNYLNQSGITKENTIIGGKFVNGENTKRKGINEYINLYSQQINDKTLKKYKMSVLFKQILS
DTESKSFVIDKLEDDSDVVTTMQSFYEQIAAFKTVEEKSIKETLSLLFDDLKAQKLDLSKIYFKND
KSLTDLSQQVEDDYSVIGTAVLEYITQQIAPKNLDNPSKKEQELIAKKTEKAKYLSLETIKLALEE
FNKHRDIDKQCRFEEILANFAAIPMIFDEIAQNKDNLAQISIKYQNQGKKDLLQASAEDDVKAIKD
LLDQTNNLLHKLKIFHISQSEDKANILDKDEHFYLVFEECYFELANIVPLYNKIRNYITQKPYSDE
KFKLNFENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKFIDFYKQ
SISKHPEWKDFGFRFSDTQRYNSIDEFYREVENQGYKLTFENISESYIDSVVNQGKLYLFQIYNKD
FSAYSKGRPNLHTLYWKALFDERNLQDVVYKLNGEAELFYRKQSIPKKITHPAKEAIANKNKDNPK
KESVFEYDLIKDKRFTEDKFFFHCPITINFKSSGANKFNDEINLLLKEKANDVHILSIDRGERHLA
YYTLVDGKGNIIKQDTFNIIGNDRMKTNYHDKLAAIEKDRDSARKDWKKINNIKEMKEGYLSQVVH
EIAKLVIEYNAIVVFEDLNFGFKRGRFKVEKQVYQKLEKMLIEKLNYLVFKDNEFDKTGGVLRAYQ
LTAPFETFKKMGKQTGIIYYVPAGFTSKICPVTGFVNQLYPKYESVSKSQEFFSKFDKICYNLDKG
YFEFSFDYKNFGDKAAKGKWTIASFGSRLINFRNSDKNHNWDTREVYPTKELEKLLKDYSIEYGHG
ECIKAAICGESDKKFFAKLTSVLNTILQMRNSKTGTELDYLISPVADVNGNFFDSRQAPKNMPQDA
DANGAYHIGLKGLMLLGRIKNNQEGKKLNLVIKNEEYFEFVQNRNN
Francisella novicida Cpf1 D917A (SEQ ID NO: 60)
(A917, E1006, and D1255 are bolded and underlined)
(SEQ ID NO: 60)
MSIYQEFVNKYSLSKTLRFELIPQGKTLENIKARGLILDDEKRAKDYKKAKQIIDKYHQFFIEEIL
SSVCISEDLLQNYSDVYFKLKKSDDDNLQKDFKSAKDTIKKQISEYIKDSEKFKNLFNQNLIDAKK
GQESDLILWLKQSKDNGIELFKANSDITDIDEALEIIKSFKGWTTYFKGFHENRKNVYSSNDIPTS
IIYRIVDDNLPKFLENKAKYESLKDKAPEAINYEQIKKDLAEELTFDIDYKTSEVNQRVFSLDEVF
EIANFNNYLNQSGITKENTIIGGKFVNGENTKRKGINEYINLYSQQINDKTLKKYKMSVLFKQILS
DTESKSFVIDKLEDDSDVVTTMQSFYEQIAAFKTVEEKSIKETLSLLFDDLKAQKLDLSKIYFKND
KSLTDLSQQVFDDYSVIGTAVLEYITQQIAPKNLDNPSKKEQELIAKKTEKAKYLSLETIKLALEE
FNKHRDIDKQCRFEEILANFAAIPMIFDEIAQNKDNLAQISIKYQNQGKKDLLQASAEDDVKAIKD
LLDQTNNLLHKLKIFHISQSEDKANILDKDEHFYLVFEECYFELANIVPLYNKIRNYITQKPYSDE
KFKLNFENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKFIDFYKQ
SISKHPEWKDFGFRFSDTQRYNSIDEFYREVENQGYKLTFENISESYIDSVVNQGKLYLFQIYNKD
FSAYSKGRPNLHTLYWKALFDERNLQDVVYKLNGEAELFYRKQSIPKKITHPAKEAIANKNKDNPK
KESVFEYDLIKDKRFTEDKFFFHCPITINFKSSGANKENDEINLLLKEKANDVHILSIARGERHLA
YYTLVDGKGNIIKQDTFNIIGNDRMKTNYHDKLAAIEKDRDSARKDWKKINNIKEMKEGYLSQVVH
EIAKLVIEYNAIVVFEDLNFGFKRGRFKVEKQVYQKLEKMLIEKLNYLVFKDNEFDKTGGVLRAYQ
LTAPFETFKKMGKQTGIIYYVPAGFTSKICPVTGFVNQLYPKYESVSKSQEFFSKFDKICYNLDKG
YFEFSFDYKNFGDKAAKGKWTIASFGSRLINFRNSDKNHNWDTREVYPTKELEKLLKDYSIEYGHG
ECIKAAICGESDKKFFAKLTSVLNTILQMRNSKTGTELDYLISPVADVNGNFFDSRQAPKNMPQDA
DANGAYHIGLKGLMLLGRIKNNQEGKKLNLVIKNEEYFEFVQNRNN
Francisella novicida Cpf1 E1006A (SEQ ID NO: 61)
(D917, A1006, and D1255 are bolded and underlined)
(SEQ ID NO: 61)
MSIYQEFVNKYSLSKTLRFELIPQGKTLENIKARGLILDDEKRAKDYKKAKQIIDKYHQFFIEEIL
SSVCISEDLLQNYSDVYFKLKKSDDDNLQKDFKSAKDTIKKQISEYIKDSEKFKNLFNQNLIDAKK
GQESDLILWLKQSKDNGIELFKANSDITDIDEALEIIKSFKGWTTYFKGFHENRKNVYSSNDIPTS
IIYRIVDDNLPKFLENKAKYESLKDKAPEAINYEQIKKDLAEELTFDIDYKTSEVNQRVFSLDEVF
EIANFNNYLNQSGITKENTIIGGKFVNGENTKRKGINEYINLYSQQINDKTLKKYKMSVLFKQILS
DTESKSFVIDKLEDDSDVVTTMQSFYEQIAAFKTVEEKSIKETLSLLFDDLKAQKLDLSKIYFKND
KSLTDLSQQVEDDYSVIGTAVLEYITQQIAPKNLDNPSKKEQELIAKKTEKAKYLSLETIKLALEE
FNKHRDIDKQCRFEEILANFAAIPMIFDEIAQNKDNLAQISIKYQNQGKKDLLQASAEDDVKAIKD
LLDQTNNLLHKLKIFHISQSEDKANILDKDEHFYLVFEECYFELANIVPLYNKIRNYITQKPYSDE
KFKLNFENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKFIDFYKQ
SISKHPEWKDFGFRFSDTQRYNSIDEFYREVENQGYKLTFENISESYIDSVVNQGKLYLFQIYNKD
FSAYSKGRPNLHTLYWKALFDERNLQDVVYKLNGEAELFYRKQSIPKKITHPAKEAIANKNKDNPK
KESVFEYDLIKDKRFTEDKFFFHCPITINFKSSGANKENDEINLLLKEKANDVHILSIDRGERHLA
YYTLVDGKGNIIKQDTFNIIGNDRMKTNYHDKLAAIEKDRDSARKDWKKINNIKEMKEGYLSQVVH
EIAKLVIEYNAIVVFADLNFGFKRGRFKVEKQVYQKLEKMLIEKLNYLVFKDNEFDKTGGVLRAYQ
LTAPFETFKKMGKQTGIIYYVPAGFTSKICPVTGFVNQLYPKYESVSKSQEFFSKFDKICYNLDKG
YFEFSFDYKNFGDKAAKGKWTIASFGSRLINFRNSDKNHNWDTREVYPTKELEKLLKDYSIEYGHG
ECIKAAICGESDKKFFAKLTSVLNTILQMRNSKTGTELDYLISPVADVNGNFFDSRQAPKNMPQDA
DANGAYHIGLKGLMLLGRIKNNQEGKKLNLVIKNEEYFEFVQNRNN
Francisella novicida Cpf1 D1255A (SEQ ID NO: 62)
(D917, E1006, and A1255 are bolded and underlined)
(SEQ ID NO: 62)
MSIYQEFVNKYSLSKTLRFELIPQGKTLENIKARGLILDDEKRAKDYKKAKQIIDKYHQFFIEEIL
SSVCISEDLLQNYSDVYFKLKKSDDDNLQKDFKSAKDTIKKQISEYIKDSEKFKNLFNQNLIDAKK
GQESDLILWLKQSKDNGIELFKANSDITDIDEALEIIKSFKGWTTYFKGFHENRKNVYSSNDIPTS
IIYRIVDDNLPKFLENKAKYESLKDKAPEAINYEQIKKDLAEELTFDIDYKTSEVNQRVFSLDEVF
EIANFNNYLNQSGITKENTIIGGKFVNGENTKRKGINEYINLYSQQINDKTLKKYKMSVLFKQILS
DTESKSFVIDKLEDDSDVVTTMQSFYEQIAAFKTVEEKSIKETLSLLFDDLKAQKLDLSKIYFKND
KSLTDLSQQVEDDYSVIGTAVLEYITQQIAPKNLDNPSKKEQELIAKKTEKAKYLSLETIKLALEE
FNKHRDIDKQCRFEEILANFAAIPMIFDEIAQNKDNLAQISIKYQNQGKKDLLQASAEDDVKAIKD
LLDQTNNLLHKLKIFHISQSEDKANILDKDEHFYLVFEECYFELANIVPLYNKIRNYITQKPYSDE
KFKLNFENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKFIDFYKQ
SISKHPEWKDFGFRFSDTQRYNSIDEFYREVENQGYKLTFENISESYIDSVVNQGKLYLFQIYNKD
FSAYSKGRPNLHTLYWKALFDERNLQDVVYKLNGEAELFYRKQSIPKKITHPAKEAIANKNKDNPK
KESVFEYDLIKDKRFTEDKFFFHCPITINFKSSGANKENDEINLLLKEKANDVHILSIDRGERHLA
YYTLVDGKGNIIKQDTFNIIGNDRMKTNYHDKLAAIEKDRDSARKDWKKINNIKEMKEGYLSQVVH
EIAKLVIEYNAIVVFEDLNFGFKRGRFKVEKQVYQKLEKMLIEKLNYLVFKDNEFDKTGGVLRAYQ
LTAPFETFKKMGKQTGIIYYVPAGFTSKICPVTGFVNQLYPKYESVSKSQEFFSKFDKICYNLDKG
YFEFSFDYKNFGDKAAKGKWTIASFGSRLINFRNSDKNHNWDTREVYPTKELEKLLKDYSIEYGHG
ECIKAAICGESDKKFFAKLTSVLNTILQMRNSKTGTELDYLISPVADVNGNFFDSRQAPKNMPQDA
AANGAYHIGLKGLMLLGRIKNNQEGKKLNLVIKNEEYFEFVQNRNN
Francisella novicida Cpf1 D917A/E1006A (SEQ ID NO: 63)
(A917, A1006, and D1255 are bolded and underlined)
(SEQ ID NO: 63)
MSIYQEFVNKYSLSKTLRFELIPQGKTLENIKARGLILDDEKRAKDYKKAKQIIDKYHQFFIEEIL
SSVCISEDLLQNYSDVYFKLKKSDDDNLQKDFKSAKDTIKKQISEYIKDSEKFKNLFNQNLIDAKK
GQESDLILWLKQSKDNGIELFKANSDITDIDEALEIIKSFKGWTTYFKGFHENRKNVYSSNDIPTS
IIYRIVDDNLPKFLENKAKYESLKDKAPEAINYEQIKKDLAEELTFDIDYKTSEVNQRVFSLDEVF
EIANFNNYLNQSGITKENTIIGGKFVNGENTKRKGINEYINLYSQQINDKTLKKYKMSVLFKQILS
DTESKSFVIDKLEDDSDVVTTMQSFYEQIAAFKTVEEKSIKETLSLLFDDLKAQKLDLSKIYFKND
KSLTDLSQQVEDDYSVIGTAVLEYITQQIAPKNLDNPSKKEQELIAKKTEKAKYLSLETIKLALEE
FNKHRDIDKQCRFEEILANFAAIPMIFDEIAQNKDNLAQISIKYQNQGKKDLLQASAEDDVKAIKD
LLDQTNNLLHKLKIFHISQSEDKANILDKDEHFYLVFEECYFELANIVPLYNKIRNYITQKPYSDE
KFKLNFENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKFIDFYKQ
SISKHPEWKDFGFRFSDTQRYNSIDEFYREVENQGYKLTFENISESYIDSVVNQGKLYLFQIYNKD
FSAYSKGRPNLHTLYWKALFDERNLQDVVYKLNGEAELFYRKQSIPKKITHPAKEAIANKNKDNPK
KESVFEYDLIKDKRFTEDKFFFHCPITINFKSSGANKENDEINLLLKEKANDVHILSIARGERHLA
YYTLVDGKGNIIKQDTFNIIGNDRMKTNYHDKLAAIEKDRDSARKDWKKINNIKEMKEGYLSQVVH
EIAKLVIEYNAIVVFADLNFGFKRGRFKVEKQVYQKLEKMLIEKLNYLVFKDNEFDKTGGVLRAYQ
LTAPFETFKKMGKQTGIIYYVPAGFTSKICPVTGFVNQLYPKYESVSKSQEFFSKFDKICYNLDKG
YFEFSFDYKNFGDKAAKGKWTIASFGSRLINFRNSDKNHNWDTREVYPTKELEKLLKDYSIEYGHG
ECIKAAICGESDKKFFAKLTSVLNTILQMRNSKTGTELDYLISPVADVNGNFFDSRQAPKNMPQDA
DANGAYHIGLKGLMLLGRIKNNQEGKKLNLVIKNEEYFEFVQNRNN
Francisella novicida Cpf1 D917A/D1255A (SEQ ID NO: 64)
(A917, E1006, and A1255 are bolded and underlined)
(SEQ ID NO: 64)
MSIYQEFVNKYSLSKTLRFELIPQGKTLENIKARGLILDDEKRAKDYKKAKQIIDKYHQFFIEEIL
SSVCISEDLLQNYSDVYFKLKKSDDDNLQKDFKSAKDTIKKQISEYIKDSEKFKNLFNQNLIDAKK
GQESDLILWLKQSKDNGIELFKANSDITDIDEALEIIKSFKGWTTYFKGFHENRKNVYSSNDIPTS
IIYRIVDDNLPKFLENKAKYESLKDKAPEAINYEQIKKDLAEELTFDIDYKTSEVNQRVFSLDEVF
EIANFNNYLNQSGITKENTIIGGKFVNGENTKRKGINEYINLYSQQINDKTLKKYKMSVLFKQILS
DTESKSFVIDKLEDDSDVVTTMQSFYEQIAAFKTVEEKSIKETLSLLFDDLKAQKLDLSKIYFKND
KSLTDLSQQVEDDYSVIGTAVLEYITQQIAPKNLDNPSKKEQELIAKKTEKAKYLSLETIKLALEE
FNKHRDIDKQCRFEEILANFAAIPMIFDEIAQNKDNLAQISIKYQNQGKKDLLQASAEDDVKAIKD
LLDQTNNLLHKLKIFHISQSEDKANILDKDEHFYLVFEECYFELANIVPLYNKIRNYITQKPYSDE
KFKLNFENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKFIDFYKQ
SISKHPEWKDFGFRFSDTQRYNSIDEFYREVENQGYKLTFENISESYIDSVVNQGKLYLFQIYNKD
FSAYSKGRPNLHTLYWKALFDERNLQDVVYKLNGEAELFYRKQSIPKKITHPAKEAIANKNKDNPK
KESVFEYDLIKDKRFTEDKFFFHCPITINFKSSGANKENDEINLLLKEKANDVHILSIARGERHLA
YYTLVDGKGNIIKQDTFNIIGNDRMKTNYHDKLAAIEKDRDSARKDWKKINNIKEMKEGYLSQVVH
EIAKLVIEYNAIVVFEDLNFGFKRGRFKVEKQVYQKLEKMLIEKLNYLVFKDNEFDKTGGVLRAYQ
LTAPFETFKKMGKQTGIIYYVPAGFTSKICPVTGFVNQLYPKYESVSKSQEFFSKFDKICYNLDKG
YFEFSFDYKNFGDKAAKGKWTIASFGSRLINFRNSDKNHNWDTREVYPTKELEKLLKDYSIEYGHG
ECIKAAICGESDKKFFAKLTSVLNTILQMRNSKTGTELDYLISPVADVNGNFFDSRQAPKNMPQDA
AANGAYHIGLKGLMLLGRIKNNQEGKKLNLVIKNEEYFEFVQNRNN
Francisella novicida Cpf1 E1006A/D1255A (SEQ ID NO: 65)
(D917, A1006, and A1255 are bolded and underlined)
(SEQ ID NO: 65)
MSIYQEFVNKYSLSKTLRFELIPQGKTLENIKARGLILDDEKRAKDYKKAKQIIDKYHQFFIEEIL
SSVCISEDLLQNYSDVYFKLKKSDDDNLQKDFKSAKDTIKKQISEYIKDSEKFKNLFNQNLIDAKK
GQESDLILWLKQSKDNGIELFKANSDITDIDEALEIIKSFKGWTTYFKGFHENRKNVYSSNDIPTS
IIYRIVDDNLPKFLENKAKYESLKDKAPEAINYEQIKKDLAEELTFDIDYKTSEVNQRVFSLDEVF
EIANFNNYLNQSGITKENTIIGGKFVNGENTKRKGINEYINLYSQQINDKTLKKYKMSVLFKQILS
DTESKSFVIDKLEDDSDVVTTMQSFYEQIAAFKTVEEKSIKETLSLLFDDLKAQKLDLSKIYFKND
KSLTDLSQQVEDDYSVIGTAVLEYITQQIAPKNLDNPSKKEQELIAKKTEKAKYLSLETIKLALEE
FNKHRDIDKQCRFEEILANFAAIPMIFDEIAQNKDNLAQISIKYQNQGKKDLLQASAEDDVKAIKD
LLDQTNNLLHKLKIFHISQSEDKANILDKDEHFYLVFEECYFELANIVPLYNKIRNYITQKPYSDE
KFKLNFENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKFIDFYKQ
SISKHPEWKDFGFRFSDTQRYNSIDEFYREVENQGYKLTFENISESYIDSVVNQGKLYLFQIYNKD
FSAYSKGRPNLHTLYWKALFDERNLQDVVYKLNGEAELFYRKQSIPKKITHPAKEAIANKNKDNPK
KESVFEYDLIKDKRFTEDKFFFHCPITINFKSSGANKENDEINLLLKEKANDVHILSIDRGERHLA
YYTLVDGKGNIIKQDTFNIIGNDRMKTNYHDKLAAIEKDRDSARKDWKKINNIKEMKEGYLSQVVH
EIAKLVIEYNAIVVFADLNFGFKRGRFKVEKQVYQKLEKMLIEKLNYLVFKDNEFDKTGGVLRAYQ
LTAPFETFKKMGKQTGIIYYVPAGFTSKICPVTGFVNQLYPKYESVSKSQEFFSKFDKICYNLDKG
YFEFSFDYKNFGDKAAKGKWTIASFGSRLINFRNSDKNHNWDTREVYPTKELEKLLKDYSIEYGHG
ECIKAAICGESDKKFFAKLTSVLNTILQMRNSKTGTELDYLISPVADVNGNFFDSRQAPKNMPQDA
AANGAYHIGLKGLMLLGRIKNNQEGKKLNLVIKNEEYFEFVQNRNN
Francisella novicida Cpf1 D917A/E1006A/D1255A (SEQ ID NO: 66)
(A917, A1006, and A1255 are bolded and underlined)
(SEQ ID NO: 66)
MSIYQEFVNKYSLSKTLRFELIPQGKTLENIKARGLILDDEKRAKDYKKAKQIIDKYHQFFIEEIL
SSVCISEDLLQNYSDVYFKLKKSDDDNLQKDFKSAKDTIKKQISEYIKDSEKFKNLFNQNLIDAKK
GQESDLILWLKQSKDNGIELFKANSDITDIDEALEIIKSFKGWTTYFKGFHENRKNVYSSNDIPTS
IIYRIVDDNLPKFLENKAKYESLKDKAPEAINYEQIKKDLAEELTFDIDYKTSEVNQRVFSLDEVF
EIANFNNYLNQSGITKENTIIGGKFVNGENTKRKGINEYINLYSQQINDKTLKKYKMSVLFKQILS
DTESKSFVIDKLEDDSDVVTTMQSFYEQIAAFKTVEEKSIKETLSLLFDDLKAQKLDLSKIYFKND
KSLTDLSQQVEDDYSVIGTAVLEYITQQIAPKNLDNPSKKEQELIAKKTEKAKYLSLETIKLALEE
FNKHRDIDKQCRFEEILANFAAIPMIFDEIAQNKDNLAQISIKYQNQGKKDLLQASAEDDVKAIKD
LLDQTNNLLHKLKIFHISQSEDKANILDKDEHFYLVFEECYFELANIVPLYNKIRNYITQKPYSDE
KFKLNFENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKFIDFYKQ
SISKHPEWKDFGFRFSDTQRYNSIDEFYREVENQGYKLTFENISESYIDSVVNQGKLYLFQIYNKD
FSAYSKGRPNLHTLYWKALFDERNLQDVVYKLNGEAELFYRKQSIPKKITHPAKEAIANKNKDNPK
KESVFEYDLIKDKRFTEDKFFFHCPITINFKSSGANKFNDEINLLLKEKANDVHILSIARGERHLA
YYTLVDGKGNIIKQDTFNIIGNDRMKTNYHDKLAAIEKDRDSARKDWKKINNIKEMKEGYLSQVVH
EIAKLVIEYNAIVVFADLNFGFKRGRFKVEKQVYQKLEKMLIEKLNYLVFKDNEFDKTGGVLRAYQ
LTAPFETFKKMGKQTGIIYYVPAGFTSKICPVTGFVNQLYPKYESVSKSQEFFSKFDKICYNLDKG
YFEFSFDYKNFGDKAAKGKWTIASFGSRLINFRNSDKNHNWDTREVYPTKELEKLLKDYSIEYGHG
ECIKAAICGESDKKFFAKLTSVLNTILQMRNSKTGTELDYLISPVADVNGNFFDSRQAPKNMPQDA
AANGAYHIGLKGLMLLGRIKNNQEGKKLNLVIKNEEYFEFVQNRNN

In some embodiments, the nucleic acid programmable DNA binding protein (napDNAbp) is a nucleic acid programmable DNA binding protein that does not require a canonical (NGG) PAM sequence. In some embodiments, the napDNAbp is an argonaute protein. One example of such a nucleic acid programmable DNA binding protein is an Argonaute protein from Natronobacterium gregoryi (NgAgo). NgAgo is a ssDNA-guided endonuclease. NgAgo binds 5′ phosphorylated ssDNA of ˜24 nucleotides (gDNA) to guide it to its target site and will make DNA double-strand breaks at the gDNA site. In contrast to Cas9, the NgAgo-gDNA system does not require a protospacer-adjacent motif (PAM). Using a nuclease inactive NgAgo (dNgAgo) can greatly expand the bases that may be targeted. The characterization and use of NgAgo have been described in Gao et al., Nat Biotechnol., 2016 July;34 (7): 768-73. PubMed PMID: 27136078; Swarts et al., Nature. 507 (7491) (2014): 258-61; and Swarts et al., Nucleic Acids Res. 43 (10) (2015): 5120-9, each of which is incorporated herein by reference. The sequence of Natronobacterium gregoryi Argonaute is provided in SEQ ID NO: 67.

Wild type Natronobacterium gregoryi Argonaute
(SEQ ID NO: 67)
(SEQ ID NO: 67)
MTVIDLDSTTTADELTSGHTYDISVTLTGVYDNTDEQHPRMSLAFEQDN
GERRYITLWKNTTPKDVFTYDYATGSTYIFTNIDYEVKDGYENLTATYQ
TTVENATAQEVGTTDEDETFAGGEPLDHHLDDALNETPDDAETESDSGH
VMTSFASRDQLPEWTLHTYTLTATDGAKTDTEYARRTLAYTVRQELYTD
HDAAPVATDGLMLLTPEPLGETPLDLDCGVRVEADETRTLDYTTAKDRL
LARELVEEGLKRSLWDDYLVRGIDEVLSKEPVLTCDEFDLHERYDLSVE
VGHSGRAYLHINFRHRFVPKLTLADIDDDNIYPGLRVKTTYRPRRGHIV
WGLRDECATDSLNTLGNQSVVAYHRNNQTPINTDLLDAIEAADRRVVET
RRQGHGDDAVSFPQELLAVEPNTHQIKQFASDGFHQQARSKTRLSASRC
SEKAQAFAERLDPVRLNGSTVEFSSEFFTGNNEQQLRLLYENGESVLTF
RDGARGAHPDETFSKGIVNPPESFEVAVVLPEQQADTCKAQWDTMADLL
NQAGAPPTRSETVQYDAFSSPESISLNVAGAIDPSEVDAAFVVLPPDQE
GFADLASPTETYDELKKALANMGIYSQMAYFDRFRDAKIFYTRNVALGL
LAAAGGVAFTTEHAMPGDADMFIGIDVSRSYPEDGASGQINIAATATAV
YKDGTILGHSSTRPQLGEKLQSTDVRDIMKNAILGYQQVTGESPTHIVI
HRDGFMNEDLDPATEFLNEQGVEYDIVEIRKQPQTRLLAVSDVQYDTPV
KSIAAINQNEPRATVATFGAPEYLATRDGGGLPRPIQIERVAGETDIET
LTRQVYLLSQSHIQVHNSTARLPITTAYADQASTHATKGYLVQTGAFES
NVGFL

In some embodiments, the napDNAbp is a prokaryotic homolog of an Argonaute protein. Prokaryotic homologs of Argonaute proteins are known and have been described, for example, in Makarova K., et al., “Prokaryotic homologs of Argonaute proteins are predicted to function as key components of a novel system of defense against mobile genetic elements”, Biol Direct. 2009 Aug. 25; 4:29. doi: 10.1186/1745-6150-4-29, the entire contents of which is hereby incorporated by reference. In some embodiments, the napDNAbp is a Marinitoga piczophila Argunaute (MpAgo) protein. The CRISPR-associated Marinitoga piezophila Argunaute (MpAgo) protein cleaves single-stranded target sequences using 5′-phosphorylated guides. The 5′ guides are used by all known Argonautes. The crystal structure of an MpAgo-RNA complex shows a guide strand binding site comprising residues that block 5′ phosphate interactions. This data suggests the evolution of an Argonaute subclass with noncanonical specificity for a 5′-hydroxylated guide. Sec, e.g., Kaya et al., “A bacterial Argonaute with noncanonical guide RNA specificity”, Proc Natl Acad Sci USA. 2016 Apr. 12;113 (15): 4057-62, the entire contents of which are hereby incorporated by reference). It should be appreciated that other argonaute proteins may be used, and are within the scope of this disclosure.

In some embodiments, the nucleic acid programmable DNA binding protein (napDNAbp) is a single effector of a microbial CRISPR-Cas system. Single effectors of microbial CRISPR-Cas systems include, without limitation, Cas9, Cpf1, C2c1, C2c2, and C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (C2c1, C2c2, and C2c3) have been described by Shmakov et al., “Discovery and Functional Characterization of Diverse Class 2 CRISPR Cas Systems”, Mol. Cell, 2015 Nov. 5; 60 (3): 385-397, the entire contents of which is hereby incorporated by reference. Effectors of two of the systems, C2c1 and C2c3, contain RuvC-like endonuclease domains related to Cpf1. A third system, C2c2 contains an effector with two predicated HEPN RNase domains. Production of mature CRISPR RNA is tracrRNA-independent, unlike production of CRISPR RNA by C2c1. C2c1 depends on both CRISPR RNA and tracrRNA for DNA cleavage. Bacterial C2c2 has been shown to possess a unique RNase activity for CRISPR RNA maturation distinct from its RNA-activated single-stranded RNA degradation activity. These RNase functions are different from each other and from the CRISPR RNA-processing behavior of Cpf1. See, e.g., East-Seletsky, et al., “Two distinct RNase activities of CRISPR-C2c2 enable guide-RNA processing and RNA detection”, Nature, 2016 Oct. 13;538 (7624): 270-273, the entire contents of which are hereby incorporated by reference. In vitro biochemical analysis of C2c2 in Leptotrichia shahii has shown that C2c2 is guided by a single CRISPR RNA and can be programed to cleave ssRNA targets carrying complementary protospacers. Catalytic residues in the two conserved HEPN domains mediate cleavage. Mutations in the catalytic residues generate catalytically inactive RNA-binding proteins. See e.g., Abudayych et al., “C2c2 is a single-component programmable RNA-guided RNA-targeting CRISPR effector”, Science, 2016 Aug. 5; 353 (6299), the entire contents of which are hereby incorporated by reference.

The crystal structure of Alicyclobaccillus acidoterrastris C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19;65 (2): 310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15;167 (7): 1814-1828, the entire contents of which are hereby incorporated by reference. Catalytically competent conformations of AacC2c1, both with target and non-target DNA strands, have been captured independently positioned within a single RuvC catalytic pocket, with C2c1-mediated cleavage resulting in a staggered seven-nucleotide break of target DNA. Structural comparisons between C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins provided herein may be a C2c1, a C2c2, or a C2c3 protein. In some embodiments, the napDNAbp is a C2c1 protein. In some embodiments, the napDNAbp is a C2c2 protein. In some embodiments, the napDNAbp is a C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring C2c1, C2c2, or C2c3 protein. In some embodiments, the napDNAbp is a naturally-occurring C2c1, C2c2, or C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to any one of SEQ ID NOs: 68 or 69. In some embodiments, the napDNAbp comprises an amino acid sequence of any one SEQ ID NOs: 68 or 69. It should be appreciated that C2c1, C2c2, or C2c3 from other bacterial species may also be used in accordance with the present disclosure.

C2c1 (uniprot.org/uniprot/T0D7A2#)
sp|T0D7A2|C2C1_ALIAG CRISPR-associated
endonuclease C2c1 OS = Alicyclobacillus
acidoterrestris (strain ATCC 49025/DSM 3922/CIP
106132/NCIMB 13137/GD3B) GN = c2c1 PE = 1 SV = 1
(SEQ ID NO: 68)
MAVKSIKVKLRLDDMPEIRAGLWKLHKEVNAGVRYYTEWLSLLRQENLYR
RSPNGDGEQECDKTAEECKAELLERLRARQVENGHRGPAGSDDELLQLAR
QLYELLVPQAIGAKGDAQQIARKFLSPLADKDAVGGLGIAKAGNKPRWVR
MREAGEPGWEEEKEKAETRKSADRTADVLRALADFGLKPLMRVYTDSEMS
SVEWKPLRKGQAVRTWDRDMFQQAIERMMSWESWNQRVGQEYAKLVEQKN
RFEQKNFVGQEHLVHLVNQLQQDMKEASPGLESKEQTAHYVTGRALRGSD
KVFEKWGKLAPDAPFDLYDAEIKNVQRRNTRRFGSHDLFAKLAEPEYQAL
WREDASFLTRYAVYNSILRKLNHAKMFATFTLPDATAHPIWTRFDKLGGN
LHQYTFLFNEFGERRHAIRFHKLLKVENGVAREVDDVTVPISMSEQLDNL
LPRDPNEPIALYFRDYGAEQHFTGEFGGAKIQCRRDQLAHMHRRRGARDV
YLNVSVRVQSQSEARGERRPPYAAVFRLVGDNHRAFVHFDKLSDYLAEHP
DDGKLGSEGLLSGLRVMSVDLGLRTSASISVFRVARKDELKPNSKGRVPF
FFPIKGNDNLVAVHERSQLLKLPGETESKDLRAIREERQRTLRQLRTQLA
YLRLLVRCGSEDVGRRERSWAKLIEQPVDAANHMTPDWREAFENELQKLK
SLHGICSDKEWMDAVYESVRRVWRHMGKQVRDWRKDVRSGERPKIRGYAK
DVVGGNSIEQIEYLERQYKFLKSWSFFGKVSGQVIRAEKGSRFAITLREH
IDHAKEDRLKKLADRIIMEALGYVYALDERGKGKWVAKYPPCQLILLEEL
SEYQFNNDRPPSENNQLMQWSHRGVFQELINQAQVHDLLVGTMYAAFSSR
FDARTGAPGIRCRRVPARCTQEHNPEPFPWWLNKFVVEHTLDACPLRADD
LIPTGEGEIFVSPFSAEEGDFHQIHADLNAAQNLQQRLWSDFDISQIRLR
CDWGEVDGELVLIPRLTGKRTADSYSNKVFYTNTGVTYYERERGKKRRKV
FAQEKLSEEEAELLVEADEAREKSVVLMRDPSGIINRGNWTRQKEFWSMV
NQRIEGYLVKQIRSRVPLQDSACENTGDI
C2c2 (uniprot.org/uniprot/P0DOC6)
>sp|P0DOC6|C2C2_LEPSD CRISPR-associated
endoribonuclease C2c2 OS = Leptotrichia shahii
(strain DSM 19757/CCUG 47503/CIP 107916/JCM 16776/
LB37) GN = c2c2 PE = 1 SV=1
(SEQ ID NO: 69)
MGNLFGHKRWYEVRDKKDFKIKRKVKVKRNYDGNKYILNINENNNKEKID
NNKFIRKYINYKKNDNILKEFTRKFHAGNILFKLKGKEGIIRIENNDDFL
ETEEVVLYIEAYGKSEKLKALGITKKKIIDEAIRQGITKDDKKIEIKRQE
NEEEIEIDIRDEYTNKTLNDCSIILRIIENDELETKKSIYEIFKNINMSL
YKIIEKIIENETEKVFENRYYEEHLREKLLKDDKIDVILTNFMEIREKIK
SNLEILGFVKFYLNVGGDKKKSKNKKMLVEKILNINVDLTVEDIADFVIK
ELEFWNITKRIEKVKKVNNEFLEKRRNRTYIKSYVLLDKHEKFKIERENK
KDKIVKFFVENIKNNSIKEKIEKILAEFKIDELIKKLEKELKKGNCDTEI
FGIFKKHYKVNFDSKKFSKKSDEEKELYKIIYRYLKGRIEKILVNEQKVR
LKKMEKIEIEKILNESILSEKILKRVKQYTLEHIMYLGKLRHNDIDMTTV
NTDDFSRLHAKEELDLELITFFASTNMELNKIFSRENINNDENIDFFGGD
REKNYVLDKKILNSKIKIIRDLDFIDNKNNITNNFIRKFTKIGTNERNRI
LHAISKERDLQGTQDDYNKVINIIQNLKISDEEVSKALNLDVVFKDKKNI
ITKINDIKISEENNNDIKYLPSFSKVLPEILNLYRNNPKNEPFDTIETEK
IVLNALIYVNKELYKKLILEDDLEENESKNIFLQELKKTLGNIDEIDENI
IENYYKNAQISASKGNNKAIKKYQKKVIECYIGYLRKNYEELFDFSDFKM
NIQEIKKQIKDINDNKTYERITVKTSDKTIVINDDFEYIISIFALLNSNA
VINKIRNRFFATSVWLNTSEYQNIIDILDEIMQLNTLRNECITENWNLNL
EEFIQKMKEIEKDFDDFKIQTKKEIFNNYYEDIKNNILTEFKDDINGCDV
LEKKLEKIVIFDDETKFEIDKKSNILQDEQRKLSNINKKDLKKKVDQYIK
DKDQEIKSKILCRIIFNSDFLKKYKKEIDNLIEDMESENENKFQEIYYPK
ERKNELYIYKKNLFLNIGNPNFDKIYGLISNDIKMADAKFLFNIDGKNIR
KNKISEIDAILKNLNDKLNGYSKEYKEKYIKKLKENDDFFAKNIQNKNYK
SFEKDYNRVSEYKKIRDLVEFNYLNKIESYLIDINWKLAIQMARFERDMH
YIVNGLRELGIIKLSGYNTGISRAYPKRNGSDGFYTTTAYYKFFDEESYK
KFEKICYGFGIDLSENSEINKPENESIRNYISHFYIVRNPFADYSIAEQI
DRVSNLLSYSTRYNNSTYASVFEVFKKDVNLDYDELKKKFKLIGNNDILE
RLMKPKKVSVLELESYNSDYIKNLIIELLTKIENTNDTL

Cas9 Domains with Reduced PAM Exclusivity

Some aspects of the disclosure provide Cas9 domains that have different PAM specificities. Typically. Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a 4 base region (e.g., a “deamination window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the Cas9 domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 comprises the amino acid sequence SEQ ID NO: 70. In some embodiments, the SaCas9 comprises a N579X mutation of SEQ ID NO: 70, wherein X is any amino acid except for N. In some embodiments, the SaCas9 comprises a N579A mutation of SEQ ID NO: 70. In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT PAM sequence. In some embodiments, the SaCas9 domain comprises one or more of a E781X, a N967X, and a R1014X mutation of SEQ ID NO: 70, wherein X is any amino acid. In some embodiments, the SaCas9 domain comprises one or more of a E781K, a N967K, and a R1014H mutation of SEQ ID NO: 70. In some embodiments, the SaCas9 domain comprises a E781K, a N967K, or a R1014H mutation of SEQ ID NO: 70.

In some embodiments, the Cas9 domain of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to SEQ ID NO: 70. In some embodiments, the Cas9 domain of any of the fusion proteins provided herein comprises the amino acid sequence of SEQ ID NOs: 70. In some embodiments, the Cas9 domain of any of the fusion proteins provided herein consists of the amino acid sequence of SEQ ID NOs: 70.

Exemplary SaCas9 sequence
(SEQ ID NO: 70)
KRNYILGLDIGITSVGYGIIDYETRDVIDAGVRLFKEANVENNEGRRSK
RGARRLKRRRRHRIQRVKKLLFDYNLLTDHSELSGINPYEARVKGLSQK
LSEEEFSAALLHLAKRRGVHNVNEVEEDTGNELSTKEQISRNSKALEEK
YVAELQLERLKKDGEVRGSINRFKTSDYVKEAKQLLKVQKAYHQLDQSF
IDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPT
LKQIAKEILVNEEDIKGYRVTSTGKPEFTNLKVYHDIKDITARKEIIEN
AELLDQIAKILTIYQSSEDIQEELTNLNSELTQEEIEQISNLKGYTGTH
NLSLKAINLILDELWHTNDNQIAIFNRLKLVPKKVDLSQQKEIPTTLVD
DFILSPVVKRSFIQSIKVINAIIKKYGLPNDIIIELAREKNSKDAQKMI
NEMQKRNRQTNERIEEIIRTTGKENAKYLIEKIKLHDMQEGKCLYSLEA
IPLEDLLNNPFNYEVDHIIPRSVSFDNSFNNKVLVKQEENSKKGNRTPF
QYLSSSDSKISYETFKKHILNLAKGKGRISKTKKEYLLEERDINRFSVQ
KDFINRNLVDTRYATRGLMNLLRSYFRVNNLDVKVKSINGGFTSFLRRK
WKFKKERNKGYKHHAEDALIIANADFIFKEWKKLDKAKKVMENQMFEEK
QAESMPEIETEQEYKEIFITPHQIKHIKDFKDYKYSHRVDKKPNRELIN
DTLYSTRKDDKGNTLIVNNLNGLYDKDNDKLKKLINKSPEKLLMYHHDP
QTYQKLKLIMEQYGDEKNPLYKYYEETGNYLTKYSKKDNGPVIKKIKYY
GNKLNAHLDITDDYPNSRNKVVKLSLKPYRFDVYLDNGVYKFVTVKNLD
VIKKENYYEVNSKCYEEAKKLKKISNQAEFIASFYNNDLIKINGELYRV
IGVNNDLLNRIEVNMIDITYREYLENMNDKRPPRIIKTIASKTQSIKKY
STDILGNLYEVKSKKHPQIIKKG

Residue N579 of SEQ ID NO: 70, which is underlined and in bold, may be mutated (e.g., to a A579) to yield a SaCas9 nickase.

### Nucleobase Modification Moiety/Nucleic Acid Effector Domain/Nucleic Acid Editing Domain

In various embodiments, the improved base editors provided herein comprise one or more nucleic acid effector domains. In various embodiments, the nucleic acid effector domain may be any protein, enzyme, or polypeptide (or functional fragment thereof) which is capable of modifying a DNA or RNA molecule. Nucleobase modification moieties can be naturally occurring, or can be recombinant. For example, a nucleobase modification moiety can include one or more DNA repair enzymes, for example, and an enzyme or protein involved in base excision repair (BER), nucleotide excision repair (NER), homology-dependnent recombinational repair (HR), non-homologous end-joining repair (NHEJ), microhomology end-joining repair (MMEJ), mismatch repair (MMR), direct reversal repair, or other known DNA repair pathway. A nucleobase modification moiety can have one or more types of enzymatic activities, including, but not limited to endonuclease activity, polymerase activity, ligase activity, replication activity, proofreading activity. Nucleobase modification moieties can also include DNA or RNA-modifying enzymes and/or mutagenic enzymes, such as, DNA methylases and deaminating enzymes (i.e., deaminases, including cytidine deaminases and adenosine deaminases, all defined above), which deaminate nucleobases leading in some cases to mutagenic corrections by way of normal cellular DNA repair and replication processes. The “nucleic acid effector domain” (e.g., a DNA effector domain or an RNA effector domain) as used herein may also refer to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments the nucleic acid editing domain is a deaminase (e.g., a cytidine deaminase, such as an APOBEC or an AID deaminase).

In some embodiments, the nucleic acid editing domain comprises a deaminase. In some embodiments, the nucleic acid editing domain comprises a deaminase. In some embodiments, the deaminase is a cytidine deaminase. In other embodiments, the deaminase is an adenosine deaminase. In some embodiments, the deaminase is an apolipoprotein B mRNA-editing complex (APOBEC) family deaminase. In some embodiments, the deaminase is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, an APOBEC3H deaminase, or an APOBEC4 deaminase. In some embodiments, the deaminase is an activation-induced deaminase (AID). In some embodiments, the deaminase is a Lamprey CDA1 (pmCDA1) deaminase. In some embodiments, the deaminase is from a human, chimpanzee, gorilla, monkey, cow, dog, rat, or mouse. In some embodiments, the deaminase is from a human. In some embodiments the deaminase is from a rat. In some embodiments, the deaminase is a rat APOBEC1 deaminase comprising the amino acid sequence set forth in (SEQ ID NO: 98). In some embodiments, the deaminase is a human APOBEC1 deaminase comprising the amino acid sequence set forth in (SEQ ID NO: 96). In some embodiments, the deaminase is pmCDA1 (SEQ ID NO: 103). In some embodiments, the deaminase is human APOBEC3G (SEQ ID NO: 82). In some embodiments, the deaminase is a human APOBEC3G variant of any one of (SEQ ID NOs: 104-106). In some embodiments, the deaminase is at least 80%, at least 85%, at least 90%, at least 92%, at least 95%, at least 96%, at least 97%, at least 98%, at least 99%, or at least 99.5% identical to any one of the amino acid sequences set forth in SEQ ID NOs: 4-9 or 72-106.

Some exemplary suitable nucleic-acid editing domains, e.g., deaminases and deaminase domains, that can be fused to Cas9 domains according to aspects of this disclosure are provided below. It should be understood that, in some embodiments, the active domain of the respective sequence can be used, e.g., the domain without a localizing signal (nuclear localization sequence, without nuclear export signal, cytoplasmic localizing signal).

```
Human AID:
(SEQ ID NO: 72)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL
(underline: nuclear localization sequence; double
underline: nuclear export signal)
Mouse AID:
(SEQ ID NO: 73)
MDSLLMKQKKFLYHFKNVRWAKGRHETYLCYVVKRRDSATSCSLDFGHLR
NKSGCHVELLFLRYISDWDLDPGRCYRVTWFTSWSPCYDCARHVAEFLRW
NPNLSLRIFTARLYFCEDRKAEPEGLRRLHRAGVQIGIMTFKDYFYCWNT
FVENRERTFKAWEGLHENSVRLTRQLRRILLPLYEVDDLRDAFRMLGE
(underline: nuclear localization sequence;
double underline: nuclear export signal)
Dog AID:
(SEQ ID NO: 74)
MDSLLMKQRKFLYHFKNVRWAKGRHETYLCYVVKRRDSATSFSLDFGHLR
NKSGCHVELLFLRYISDWDLDPGRCYRVTWFTSWSPCYDCARHVADFLRG
YPNLSLRIFAARLYFCEDRKAEPEGLRRLHRAGVQIAIMTFKDYFYCWNT
FVENREKTFKAWEGLHENSVRLSRQLRRILLPLYEVDDLRDAFRTLGL
(underline: nuclear localization sequence; double
underline: nuclear export signal)
Bovine AID:
(SEQ ID NO: 75)
MDSLLKKQRQFLYQFKNVRWAKGRHETYLCYVVKRRDSPTSFSLDFGHLR
NKAGCHVELLFLRYISDWDLDPGRCYRVTWFTSWSPCYDCARHVADFLRG
YPNLSLRIFTARLYFCDKERKAEPEGLRRLHRAGVQIAIMTFKDYFYCWN
TFVENHERTFKAWEGLHENSVRLSRQLRRILLPLYEVDDLRDAFRTLGL
(underline: nuclear localization sequence; double
underline: nuclear export signal)
Rat: AID:
(SEQ ID NO: 76)
MAVGSKPKAALVGPHWERERIWCFLCSTGLGTQQTGQTSRWLRPAATQDP
VSPPRSLLMKQRKFLYHFKNVRWAKGRHETYLCYVVKRRDSATSFSLDFG
YLRNKSGCHVELLFLRYISDWDLDPGRCYRVTWFTSWSPCYDCARHVADF
LRGNPNLSLRIFTARLTGWGALPAGLMSPARPSDYFYCWNTFV
ENHERTFKAWEGLHENSVRLSRRLRRILLPLYEVDDLRDAFRTLGL
(underline: nuclear localization sequence;
double underline: nuclear export signal)
Mouse APOBEC-3:
(SEQ ID NO: 77)
MGPFCLGCSHRKCYSPIRNLISQETFKFHFKNLGYAKGRKDTFLCYEVTR
KDCDSPVSLHHGVFKNKDNIHAEICFLYWFHDKVLKVLSPREEFKITWYM
SWSPCFECAEQIVRFLATHHNLSLDIFSSRLYNVQDPETQQNLCRLVQEG
AQVAAMDLYEFKKCWKKFVDNGGRRFRPWKRLLTNFRYQDSKLQEILRPC
YIPVPSSSSSTLSNICLTKGLPETRFCVEGRRMDPLSEEEFYSQFYNQRV
KHLCYYHRMKPYLCYQLEQFNGQAPLKGCLLSEKGKQHAEILFLDKIRSM
ELSQVTITCYLTWSPCPNCAWQLAAFKRDRPDLILHIYTSRLYFHWKRPF
QKGLCSLWQSGILVDVMDLPQFTDCWTNFVNPKRPFWPWKGLEIISRRTQ
RRLRRIKESWGLQDLVNDFGNLQLGPPMS
(italic: nucleic acid editing domain)
Rat APOBEC-3:
(SEQ ID NO: 78)
MGPFCLGCSHRKCYSPIRNLISQETFKFHFKNLRYAIDRKDTFLCYEVTRK
DCDSPVSLHHGVFKNKDNIHAEICFLYWFHDKVLKVLSPREEFKITWYMSW
SPCFECAEQVLRFLATHHNLSLDIFSSRLYNIRDPENQQNLCRLVQEGAQV
AAMDLYEFKKCWKKFVDNGGRRFRPWKKLLTNFRYQDSKLQEILRPCYIPV
PSSSSSTLSNICLTKGLPETRFCVERRRVHLLSEEEFYSQFYNQRVKHLCY
YHGVKPYLCYQLEQFNGQAPLKGCLLSEKGKQHAEILFLDKIRSMELSQVI
ITCYLTWSPCPNCAWQLAAFKRDRPDLILHIYTSRLYFHWKRPFQKGLCSL
WQSGILVDVMDLPQFTDCWTNFVNPKRPFWPWKGLEIISRRTQRRLHRIKE
SWGLQDLVNDFGNLQLGPPMS
(italic: nucleic acid editing domain)
Rhesus macaque APOBEC-3G:
(SEQ ID NO: 79)
MVEPMDPRTFVSNFNNRPILSGLNTVWLCCEVKTKDPSGPPLDAKIFQGKV
YSKAKYHPEMRFLRWFHKWRQLHHDQEYKVTWYVSWSPCTRCANSVATFLA
KDPKVTLTIFVARLYYFWKPDYQQALRILCQKRGGPHATMKIMNYNEFQDC
WNKFVDGRGKPFKPRNNLPKHYTLLQATLGELLRHLMDPGTFTSNFNNKPW
VSGQHETYLCYKVERLHNDTWVPLNQHRGFLRNQAPNIHGFPKGRHAELCF
LDLIPFWKLDGQQYRVTCFTSWSPCFSCAQEMAKFISNNEHVSLCIFAARI
YDDQGRYQEGLRALHRDGAKIAMMNYSEFEYCWDTFVDRQGR
PFQPWDGLDEHSQALSGRLRAI
(italic: nucleic acid editing domain; underline:
cytoplasmic localization signal)
Chimpanzee APOBEC-3G:
(SEQ ID NO: 80)
MKPHFRNPVERMYQDTFSDNFYNRPILSHRNTVWLCYEVKTKGPSRPPLDA
KIFRGQVYSKLKYHPEMRFFHWFSKWRKLHRDQEYEVTWYISWSPCTKCTR
DVATFLAEDPKVTLTIFVARLYYFWDPDYQEALRSLCQKRDGPRATMKIMN
YDEFQHCWSKFVYSQRELFEPWNNLPKYYILLHIMLGEILRHSMDPPTFTS
NFNNELWVRGRHETYLCYEVERLHNDTWVLLNQRRGFLCNQAPHKHGFLEG
RHAELCFLDVIPFWKLDLHQDYRVTCFTSWSPCFSCAQEMAKFISNNKHVS
LCIFAARIYDDQGRCQEGLRTLAKAGAKISIMTYSEFKHCWD
TFVDHQGCPFQPWDGLEEHSQALSGRLRAILQNQGN
(italic: nucleic acid editing domain; underline:
cytoplasmic localization signal)
Green monkey APOBEC-3G:
(SEQ ID NO: 81)
MNPQIRNMVEQMEPDIFVYYFNNRPILSGRNTVWLCYEVKTKDPSGPPLDA
NIFQGKLYPEAKDHPEMKFLHWFRKWRQLHRDQEYEVTWYVSWSPCTRCAN
SVATFLAEDPKVTLTIFVARLYYFWKPDYQQALRILCQERGGPHATMKIMN
YNEFQHCWNEFVDGQGKPFKPRKNLPKHYTLLHATLGELLRHVMDPGTFTS
NFNNKPWVSGQRETYLCYKVERSHNDTWVLLNQHRGFLRNQAPDRHGFPKG
RHAELCFLDLIPFWKLDDQQYRVTCFTSWSPCFSCAQKMAKFISNNKHVSL
CIFAARIYDDQGRCQEGLRTLHRDGAKIAVMNYSEFEYCWDTFVDRQGRPF
QPWDGLDEHSQALSGRLRAI
(italic: nucleic acid editing domain; underline:
cytoplasmic localization signal)
Human APOBEC-3G:
(SEQ ID NO: 82)
MKPHFRNTVERMYRDTFSYNFYNRPILSRRNTVWLCYEVKTKGPSRPPLDA
KIFRGQVYSELKYHPEMRFFHWFSKWRKLHRDQEYEVTWYISWSPCTKCTR
DMATFLAEDPKVTLTIFVARLYYFWDPDYQEALRSLCQKRDGPRATMKIMN
YDEFQHCWSKFVYSQRELFEPWNNLPKYYILLHIMLGEILRHSMDPPTFTF
NFNNEPWVRGRHETYLCYEVERMHNDTWVLLNQRRGFLCNQAPHKHGFLEG
RHAELCFLDVIPFWKLDLDQDYRVTCFTSWSPCFSCAQEMAKFISKNKHVS
LCIFTARIYDDQGRCQEGLRTLAEAGAKISIMTYSEFKHCWDTFVDHQGCP
FQPWDGLDEHSQDLSGRLRAILQNQEN
(italic: nucleic acid editing domain; underline:
cytoplasmic localization signal)
Human APOBEC-3F:
(SEQ ID NO: 83)
MKPHFRNTVERMYRDTFSYNFYNRPILSRRNTVWLCYEVKTKGPSRPRLDA
KIFRGQVYSQPEHHAEMCFLSWFCGNQLPAYKCFQITWFVSWTPCPDCVAK
LAEFLAEHPNVTLTISAARLYYYWERDYRRALCRLSQAGARVKIMDDEEFA
YCWENFVYSEGQPFMPWYKFDDNYAFLHRTLKEILRNPMEAMYPHIFYFHF
KNLRKAYGRNESWLCFTMEVVKHHSPVSWKRGVFRNQVDPETHCHAERCFL
SWFCDDILSPNTNYEVTWYTSWSPCPECAGEVAEFLARHSNVNLTIFTARL
YYFWDTDYQEGLRSLSQEGASVEIMGYKDFKYCWENFVYNDD
EPFKPWKGLKYNFLFLDSKLQEILE
(italic: nucleic acid editing domain)
Human APOBEC-3B:
(SEQ ID NO: 84)
MNPQIRNPMERMYRDTFYDNFENEPILYGRSYTWLCYEVKIKRGRSNLLWD
TGVFRGQVYFKPQYHAEMCFLSWFCGNQLPAYKCFQITWFVSWTPCPDCVA
KLAEFLSEHPNVTLTISAARLYYYWERDYRRALCRLSQAGARVTIMDYEEF
AYCWENFVYNEGQQFMPWYKFDENYAFLHRTLKEILRYLMDPDTFTFNFNN
DPLVLRRRQTYLCYEVERLDNGTWVLMDQHMGFLCNEAKNLLCGFYGRHAE
LRFLDLVPSLQLDPAQIYRVTWFISWSPCFSWGCAGEVRAFLQENTHVRLR
IFAARIYDYDPLYKEALQMLRDAGAQVSIMTYDEFEYCWDTFVYRQGCPFQ
PWDGLEEHSQALSGRLRAILQNQGN
(italic: nucleic acid editing domain)
Rat APOBEC-3B:
  (SEQ ID NO: 85)
MQPQGLGPNAGMGPVCLGCSHRRPYSPIRNPLKKLYQQTFYFHFKNVRYAW
GRKNNFLCYEVNGMDCALPVPLRQGVFRKQGHIHAELCFIYWFHDKVLRVL
SPMEEFKVTWYMSWSPCSKCAEQVARFLAAHRNLSLAIFSSRLYYYLRNPN
YQQKLCRLIQEGVHVAAMDLPEFKKCWNKFVDNDGQPFRPWMRLRINFSFY
DCKLQEIFSRMNLLREDVFYLQFNNSHRVKPVQNRYYRRKSYLCYQLERAN
GQEPLKGYLLYKKGEQHVEILFLEKMRSMELSQVRITCYLTWSPCPNCARQ
LAAFKKDHPDLILRIYTSRLYFYWRKKFQKGLCTLWRSGIHVDVMDLPQFA
DCWTNFVNPQRPFRPWNELEKNSWRIQRRLRRIKESWGL
Bovine APOBEC-3B:
  (SEQ ID NO: 86)
DGWEVAFRSGTVLKAGVLGVSMTEGWAGSGHPGQGACVWTPGTRNTMNLLR
EVLFKQQFGNQPRVPAPYYRRKTYLCYQLKQRNDLTLDRGCFRNKKQRHAE
IRFIDKINSLDLNPSQSYKIICYITWSPCPNCANELVNFITRNNHLKLEIF
ASRLYFHWIKSFKMGLQDLQNAGISVAVMTHTEFEDCWEQFVDNQSRPFQP
WDKLEQYSASIRRRLQRILTAPI
Chimpanzee APOBEC-3B:
(SEQ ID NO: 87)
MNPQIRNPMEWMYQRTFYYNFENEPILYGRSYTWLCYEVKIRRGHSNLLWD
TGVFRGQMYSQPEHHAEMCFLSWFCGNQLSAYKCFQITWFVSWTPCPDCVA
KLAKFLAEHPNVTLTISAARLYYYWERDYRRALCRLSQAGARVKIMDDEEF
AYCWENFVYNEGQPFMPWYKFDDNYAFLHRTLKEIIRHLMDPDTFTFNFNN
DPLVLRRHQTYLCYEVERLDNGTWVLMDQHMGFLCNEAKNLLCGFYGRHAE
LRFLDLVPSLQLDPAQIYRVTWFISWSPCFSWGCAGQVRAFLQENTHVRLR
IFAARIYDYDPLYKEALQMLRDAGAQVSIMTYDEFEYCWDTFVYRQGCPFQ
PWDGLEEHSQALSGRLRAILQVRASSLCMVPHRPPPPPQSPGPCLPLCSEP
PLGSLLPTGRPAPSLPFLLTASFSFPPPASLPPLPSLSLSPGHLPVPSFHS
LTSCSIQPPCSSRIRETEGWASVSKEGRDLG
Human APOBEC-3C:
(SEQ ID NO: 88)
MNPQIRNPMKAMYPGTFYFQFKNLWEANDRNETWLCFTVEGIKRRSVVSWK
TGVFRNQVDSETHCHAERCFLSWFCDDILSPNTKYQVTWYTSWSPCPDCAG
EVAEFLARHSNVNLTIFTARLYYFQYPCYQEGLRSLSQEGVAVEIMDYEDF
KYCWENFVYNDNEPFKPWKGLKTNFRLLKRRLRESLQ
(italic: nucleic acid editing domain)
Gorilla APOBEC3C:
(SEQ ID NO: 89)
MNPQIRNPMKAMYPGTFYFQFKNLWEANDRNETWLCFTVEGIKRRSVVSWK
TGVFRNQVDSETHCHAERCFLSWFCDDILSPNTNYQVTWYTSWSPCPECAG
EVAEFLARHSNVNLTIFTARLYYFQDTDYQEGLRSLSQEGVAVKIMDYKDF
KYCWENFVYNDDEPFKPWKGLKYNFRFLKRRLQEILE
(italic: nucleic acid editing domain)
Human APOBEC-3A:
(SEQ ID NO: 90)
MEASPASGPRHLMDPHIFTSNFNNGIGRHKTYLCYEVERLDNGTSVKMDQH
RGFLHNQAKNLLCGFYGRHAELRFLDLVPSLQLDPAQIYRVTWFISWSPCF
SWGCAGEVRAFLQENTHVRLRIFAARIYDYDPLYKEALQMLRDAGAQVSIM
TYDEFKHCWDTFVDHQGCPFQPWDGLDEHSQALSGRLRAILQNQGN
(italic: nucleic acid editing domain)
Rhesus macaque APOBEC-3A:
(SEQ ID NO: 91)
MDGSPASRPRHLMDPNTFTFNFNNDLSVRGRHQTYLCYEVERLDNGTWVPM
DERRGFLCNKAKNVPCGDYGCHVELRFLCEVPSWQLDPAQTYRVTWFISWS
PCFRRGCAGQVRVFLQENKHVRLRIFAARIYDYDPLYQEALRTLRDAGAQV
SIMTYEEFKHCWDTFVDRQGRPFQPWDGLDEHSQALSGRLRAILQNQGN
(italic: nucleic acid editing domain)
Bovine APOBEC-3A:
(SEQ ID NO: 92)
MDEYTFTENFNNQGWPSKTYLCYEMERLDGDATIPLDEYKGFVRNKGLDQP
EKPCHAELYFLGKIHSWNLDRNQHYRLTCFISWSPCYDCAQKLTTFLKENH
HISLHILASRIYTHNRFGCHQSGLCELQAAGARITIMTFEDFKHCWETFVD
HKGKPFQPWEGLNVKSQALCTELQAILKTQQN
(italic: nucleic acid editing domain)
Human APOBEC-3H:
  (SEQ ID NO: 93)
MALLTAETFRLQFNNKRRLRRPYYPRKALLCYQLTPQNGSTPTRGYFENKK
KCHAEICFINEIKSMGLDETQCYQVTCYLTWSPCSSCAWELVDFIKAHDHL
NLGIFASRLYYHWCKPQQKGLRLLCGSQVPVEVMGFPKFADCWENFVDHEK
PLSFNPYKMLEELDKNSRAIKRRLERIKIPGVRAQGRYMDILCDAEV
(italic: nucleic acid editing domain)
Rhesus macaque APOBEC-3H:
(SEQ ID NO: 94)
MALLTAKTFSLQFNNKRRVNKPYYPRKALLCYQLTPQNGSTPTRGHLKNKK
KDHAEIRFINKIKSMGLDETQCYQVTCYLTWSPCPSCAGELVDFIKAHRHL
NLRIFASRLYYHWRPNYQEGLLLLCGSQVPVEVMGLPEFTDCWENFVDHKE
PPSFNPSEKLEELDKNSQAIKRRLERIKSRSVDVLENGLRSLQLGPVTPSS
SIRNSR
Human APOBEC-3D:
(SEQ ID NO: 95)
MNPQIRNPMERMYRDTFYDNFENEPILYGRSYTWLCYEVKIKRGRSNLLWD
TGVFRGPVLPKRQSNHRQEVYFRFENHAEMCFLSWFCGNRLPANRRFQITW
FVSWNPCLPCVVKVTKFLAEHPNVTLTISAARLYYYRDRDWRWVLLRLHKA
GARVKIMDYEDFAYCWENFVCNEGQPFMPWYKFDDNYASLHRTLKEILRNP
MEAMYPHIFYFHFKNLLKACGRNESWLCFTMEVTKHHSAVFRKRGVFRNQV
DPETHCHAERCFLSWFCDDILSPNTNYEVTWYTSWSPCPECAGEVAEFLAR
HSNVNLTIFTARLCYFWDTDYQEGLCSLSQEGASVKIMGYKDFVSCWKNFV
YSDDEPFKPWKGLQTNFRLLKRRLREILQ
(italic: nucleic acid editing domain)
Human APOBEC-1:
  (SEQ ID NO: 96)
MTSEKGPSTGDPTLRRRIEPWEFDVFYDPRELRKEACLLYEIKWGMSRKIW
RSSGKNTTNHVEVNFIKKFTSERDFHPSMSCSITWFLSWSPCWECSQAIRE
FLSRHPGVTLVIYVARLFWHMDQQNRQGLRDLVNSGVTIQIMRASEYYHCW
RNFVNYPPGDEAHWPQYPPLWMMLYALELHCIILSLPPCLKISRRWQNHLT
FFRLHLQNCHYQTIPPHILLATGLIHPSVAWR
Mouse APOBEC-1:
(SEQ ID NO: 97)
MSSETGPVAVDPTLRRRIEPHEFEVFFDPRELRKETCLLYEINWGGRHSVW
RHTSQNTSNHVEVNFLEKFTTERYFRPNTRCSITWFLSWSPCGECSRAITE
FLSRHPYVTLFIYIARLYHHTDQRNRQGLRDLISSGVTIQIMTEQEYCYCW
RNFVNYPPSNEAYWPRYPHLWVKLYVLELYCIILGLPPCLKILRRKQPQLT
FFTITLQTCHYQRIPPHLLWATGLK
Rat APOBEC-1:
(SEQ ID NO: 98)
MSSETGPVAVDPTLRRRIEPHEFEVFFDPRELRKETCLLYEINWGGRHSIW
RHTSQNTNKHVEVNFIEKFTTERYFCPNTRCSITWFLSWSPCGECSRAITE
FLSRYPHVTLFIYIARLYHHADPRNRQGLRDLISSGVTIQIMTEQESGYCW
RNFVNYSPSNEAHWPRYPHLWVRLYVLELYCIILGLPPCLNILRRKQPQLT
FFTIALQSCHYQRLPPHILWATGLK
Human APOBEC-2:
(SEQ ID NO: 99)
MAQKEEAAVATEAASQNGEDLENLDDPEKLKELIELPPFEIVTGERLPANF
FKFQFRNVEYSSGRNKTFLCYVVEAQGKGGQVQASRGYLEDEHAAAHAEEA
FFNTILPAFDPALRYNVTWYVSSSPCAACADRIIKTLSKTKNLRLLILVGR
LFMWEEPEIQAALKKLKEAGCKLRIMKPQDFEYVWQNFVEQEEGESKAFQP
WEDIQENFLYYEEKLADILK
Mouse APOBEC-2:
(SEQ ID NO: 100)
MAQKEEAAEAAAPASQNGDDLENLEDPEKLKELIDLPPFEIVTGVRLPVNF
FKFQFRNVEYSSGRNKTFLCYVVEVQSKGGQAQATQGYLEDEHAGAHAEEA
FFNTILPAFDPALKYNVTWYVSSSPCAACADRILKTLSKTKNLRLLILVSR
LFMWEEPEVQAALKKLKEAGCKLRIMKPQDFEYIWQNFVEQEEGESKAFEP
WEDIQENFLYYEEKLADILK
Rat APOBEC-2:
(SEQ ID NO: 101)
MAQKEEAAEAAAPASQNGDDLENLEDPEKLKELIDLPPFEIVTGVRLPVNF
FKFQFRNVEYSSGRNKTFLCYVVEAQSKGGQVQATQGYLEDEHAGAHAEEA
FFNTILPAFDPALKYNVTWYVSSSPCAACADRILKTLSKTKNLRLLILVSR
LFMWEEPEVQAALKKLKEAGCKLRIMKPQDFEYLWQNFVEQEEGESKAFEP
WEDIQENFLYYEEKLADILK
Bovine APOBEC-2:
  (SEQ ID NO: 102)
MAQKEEAAAAAEPASQNGEEVENLEDPEKLKELIELPPFEIVTGERLPAHY
FKFQFRNVEYSSGRNKTFLCYVVEAQSKGGQVQASRGYLEDEHATNHAEEA
FFNSIMPTFDPALRYMVTWYVSSSPCAACADRIVKTLNKTKNLRLLILVGR
LFMWEEPEIQAALRKLKEAGCRLRIMKPQDFEYIWQNFVEQEEGESKAFEP
WEDIQENFLYYEEKLADILK
Petromyzon marinus CDA1 (pmCDA1)
  (SEQ ID NO: 103)
MTDAEYVRIHEKLDIYTFKKQFFNNKKSVSHRCYVLFELKRRGERRACFWG
YAVNKPQSGTERGIHAEIFSIRKVEEYLRDNPGQFTINWYSSWSPCADCAE
KILEWYNQELRGNGHTLKIWACKLYYEKNARNQIGLWNLRDNGVGLNVMVS
EHYQCCRKIFIQSSHNQLNENRWLEKTLKRAEKRRSELSIMIQVKILHTTK
SPAV
Human APOBEC3G D316R_D317R
(SEQ ID NO: 104)
MKPHFRNTVERMYRDTFSYNFYNRPILSRRNTVWLCYEVKTKGPSRPPLDA
KIFRGQVYSELKYHPEMRFFHWFSKWRKLHRDQEYEVTWYISWSPCTKCTR
DMATFLAEDPKVTLTIFVARLYYFWDPDYQEALRSLCQKRDGPRATMKIMN
YDEFQHCWSKFVYSQRELFEPWNNLPKYYILLHIMLGEILRHSMDPPTFTF
NFNNEPWVRGRHETYLCYEVERMHNDTWVLLNQRRGFLCNQAPHKHGFLEG
RHAELCFLDVIPFWKLDLDQDYRVTCFTSWSPCFSCAQEMAKFISKNKHVS
LCIFTARIYRRQGRCQEGLRTLAEAGAKISIMTYSEFKHCWDTFVDHQGCP
FQPWDGLDEHSQDLSGRLRAILQNQEN
Human APOBEC3G chain A
(SEQ ID NO: 105)
MDPPTFTFNFNNEPWVRGRHETYLCYEVERMHNDTWVLLNQRRGFLCNQAP
HKHGFLEGRHAELCFLDVIPFWKLDLDQDYRVTCFTSWSPCFSCAQEMAKF
ISKNKHVSLCIFTARIYDDQGRCQEGLRTLAEAGAKISIMTYSEFKHCWDT
FVDHQGCPFQPWDGLDEHSQDLSGRLRAILQ
Human APOBEC3G chain A D120R_D121R
(SEQ ID NO: 106)
MDPPTFTFNFNNEPWVRGRHETYLCYEVERMHNDTWVLLNQRRGFLCNQAP
HKHGFLEGRHAELCFLDVIPFWKLDLDQDYRVTCFTSWSPCFSCAQEMAKF
ISKNKHVSLCIFTARIYRRQGRCQEGLRTLAEAGAKISIMTYSEFKHCWDT
FVDHQGCPFQPWDGLDEHSQDLSGRLRAILQ
```

### Cytidine Deaminases

Some aspects of the disclosure provide cytidine deaminases.

In some embodiments, second protein comprises a nucleic acid editing domain. In some embodiments, the nucleic acid editing domain can catalyze a C to U base change. In some embodiments, the nucleic acid editing domain is a deaminase domain. In some embodiments, the deaminase is a cytidine deaminase or a cytidine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a vertebrate deaminase. In some embodiments, the deaminase is an invertebrate deaminase. In some embodiments, the deaminase is a human, chimpanzee, gorilla, monkey, cow, dog, rat, or mouse deaminase. In some embodiments, the deaminase is a human deaminase. In some embodiments, the deaminase is a rat deaminase, e.g., rAPOBEC1. In some embodiments, the deaminase is a Petromyzon marinus cytidine deaminase 1 (pmCDA1). In some embodiments, the deminase is a human APOBEC3G (SEQ ID NO: 82). In some embodiments, the deaminase is a fragment of the human APOBEC3G (SEQ ID NO: 105). In some embodiments, the deaminase is a human APOBEC3G variant comprising a D316R_D317R mutation (SEQ ID NO: 104). In some embodiments, the deaminase is a frantment of the human APOBEC3G and comprising mutations corresponding to the D316R_D317R mutations in SEQ ID NO: 82 (SEQ ID NO: 106).

In some embodiments, the nucleic acid editing domain is at least 80%, at least 85%, at least 90%, at least 92%, at least 95%, at least 96%, at least 97%, at least 98%, at least 99%, or at least 99.5% identical to the deaminase domain of any one of SEQ ID NOs: 4-9, or 72-106. In some embodiments, the nucleic acid editing domain comprises the amino acid sequence of any one of SEQ ID NOs: 4-9, or 72-106.

Deaminase Domains that Modulate the Editing Window of Base Editors

Some aspects of the disclosure are based on the recognition that modulating the deaminase domain catalytic activity of any of the fusion proteins provided herein, for example by making point mutations in the deaminase domain, affect the processivity of the fusion proteins (e.g., base editors). For example, mutations that reduce, but do not eliminate, the catalytic activity of a deaminase domain within a base editing fusion protein can make it less likely that the deaminase domain will catalyze the deamination of a residue adjacent to a target residue, thereby narrowing the deamination window. The ability to narrow the deaminataion window may prevent unwanted deamination of residues adjacent of specific target residues, which may decrease or prevent off-target effects.

In some embodiments, any of the fusion proteins provided herein comprise a deaminase domain (e.g., a cytidine deaminase domain) that has reduced catalytic deaminase activity. In some embodiments, any of the fusion proteins provided herein comprise a deaminase domain (e.g., a cytidine deaminase domain) that has a reduced catalytic deaminase activity as compared to an appropriate control. For example, the appropriate control may be the deaminase activity of the deaminase prior to introducing one or more mutations into the deaminase. In other embodiments, the appropriate control may be a wild-type deaminase. In some embodiments, the appropriate control is a wild-type apolipoprotein B mRNA-editing complex (APOBEC) family deaminase. In some embodiments, the appropriate control is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, or an APOBEC3H deaminase. In some embodiments, the appropriate control is an activation induced deaminase (AID). In some embodiments, the appropriate control is a cytidine deaminase 1 from Petromyzon marinus (pmCDA1). In some embodiments, the deaminse domain may be a deaminase domain that has at least 1%, at least 5%, at least 15%, at least 20%, at least 25%, at least 30%, at least 40%, at least 50%, at least 60%, at lest 70%, at least 80%, at least 90%, or at least 95% less catalytic deaminase activity as compared to an appropriate control.

In some embodiments, any of the fusion proteins provided herein comprise an APOBEC deaminase comprising one or more mutations selected from the group consisting of H121X, H122X, R126X, R126X, R118X, W90X, W90X, and R132X of rAPOBEC1 (SEQ ID NO: 98), or one or more corresponding mutations in another APOBEC deaminase, wherein X is any amino acid. In some embodiments, any of the fusion proteins provided herein comprise an APOBEC deaminase comprising one or more mutations selected from the group consisting of H121R, H122R, R126A, R126E, R118A, W90A, W90Y, and R132E of rAPOBEC1 (SEQ ID NO: 98), or one or more corresponding mutations in another APOBEC deaminase.

In some embodiments, any of the fusion proteins provided herein comprise an APOBEC deaminase comprising one or more mutations selected from the group consisting of D316X, D317X, R320X, R320X, R313X, W285X, W285X, R326X of hAPOBEC3G (SEQ ID NO: 82), or one or more corresponding mutations in another APOBEC deaminase, wherein X is any amino acid. In some embodiments, any of the fusion proteins provided herein comprise an APOBEC deaminase comprising one or more mutations selected from the group consisting of D316R, D317R, R320A, R320E, R313A, W285A, W285Y, R326E of hAPOBEC3G (SEQ ID NO: 82), or one or more corresponding mutations in another APOBEC deaminase.

In some embodiments, any of the fusion proteins provided herein comprise an APOBEC deaminase comprising a H121R and a H122Rmutation of rAPOBEC1 (SEQ ID NO: 98), or one or more corresponding mutations in another APOBEC deaminase. In some embodiments, any of the fusion proteins provided herein comprise an APOBEC deaminase comprising a R126A mutation of rAPOBEC1 (SEQ ID NO: 98), or one or more corresponding mutations in another APOBEC deaminase. In some embodiments, any of the fusion proteins provided herein comprise an APOBEC deaminase comprising a R126E mutation of rAPOBEC1 (SEQ ID NO: 98), or one or more corresponding mutations in another APOBEC deaminase. In some embodiments, any of the fusion proteins provided herein comprise an APOBEC deaminase comprising a R118A mutation of rAPOBEC1 (SEQ ID NO: 98), or one or more corresponding mutations in another APOBEC deaminase. In some embodiments, any of the fusion proteins provided herein comprise an APOBEC deaminase comprising a W90A mutation of rAPOBEC1 (SEQ ID NO: 98), or one or more corresponding mutations in another APOBEC deaminase. In some embodiments, any of the fusion proteins provided herein comprise an APOBEC deaminase comprising a W90Y mutation of rAPOBEC1 (SEQ ID NO: 98), or one or more corresponding mutations in another APOBEC deaminase. In some embodiments, any of the fusion proteins provided herein comprise an APOBEC deaminase comprising a R132E mutation of rAPOBEC1 (SEQ ID NO: 98), or one or more corresponding mutations in another APOBEC deaminase. In some embodiments, any of the fusion proteins provided herein comprise an APOBEC deaminase comprising a W90Y and a R126E mutation of rAPOBEC1 (SEQ ID NO: 98), or one or more corresponding mutations in another APOBEC deaminase. In some embodiments, any of the fusion proteins provided herein comprise an APOBEC deaminase comprising a R126E and a R132E mutation of rAPOBEC1 (SEQ ID NO: 98), or one or more corresponding mutations in another APOBEC deaminase. In some embodiments, any of the fusion proteins provided herein comprise an APOBEC deaminase comprising a W90Y and a R132E mutation of rAPOBEC1 (SEQ ID NO: 98), or one or more corresponding mutations in another APOBEC deaminase. In some embodiments, any of the fusion proteins provided herein comprise an APOBEC deaminase comprising a W90Y, R126E, and R132E mutation of rAPOBEC1 (SEQ ID NO: 98), or one or more corresponding mutations in another APOBEC deaminase.

In some embodiments, any of the fusion proteins provided herein comprise an APOBEC deaminase comprising a D316R and a D317R mutation of hAPOBEC3G (SEQ ID NO: 82), or one or more corresponding mutations in another APOBEC deaminase. In some embodiments, any of the fusion proteins provided herein comprise an APOBEC deaminase comprising a R320A mutation of hAPOBEC3G (SEQ ID NO: 82), or one or more corresponding mutations in another APOBEC deaminase. In some embodiments, any of the fusion proteins provided herein comprise an APOBEC deaminase comprising a R320E mutation of hAPOBEC3G (SEQ ID NO: 82), or one or more corresponding mutations in another APOBEC deaminase. In some embodiments, any of the fusion proteins provided herein comprise an APOBEC deaminase comprising a R313A mutation of hAPOBEC3G (SEQ ID NO: 82), or one or more corresponding mutations in another APOBEC deaminase. In some embodiments, any of the fusion proteins provided herein comprise an APOBEC deaminase comprising a W285A mutation of hAPOBEC3G (SEQ ID NO: 82), or one or more corresponding mutations in another APOBEC deaminase. In some embodiments, any of the fusion proteins provided herein comprise an APOBEC deaminase comprising a W285Y mutation of hAPOBEC3G (SEQ ID NO: 82), or one or more corresponding mutations in another APOBEC deaminase. In some embodiments, any of the fusion proteins provided herein comprise an APOBEC deaminase comprising a R326E mutation of hAPOBEC3G (SEQ ID NO: 82), or one or more corresponding mutations in another APOBEC deaminase. In some embodiments, any of the fusion proteins provided herein comprise an APOBEC deaminase comprising a W285Y and a R320E mutation of hAPOBEC3G (SEQ ID NO: 82), or one or more corresponding mutations in another APOBEC deaminase. In some embodiments, any of the fusion proteins provided herein comprise an APOBEC deaminase comprising a R320E and a R326E mutation of hAPOBEC3G (SEQ ID NO: 82), or one or more corresponding mutations in another APOBEC deaminase. In some embodiments, any of the fusion proteins provided herein comprise an APOBEC deaminase comprising a W285Y and a R326E mutation of hAPOBEC3G (SEQ ID NO: 82), or one or more corresponding mutations in another APOBEC deaminase. In some embodiments, any of the fusion proteins provided herein comprise an APOBEC deaminase comprising a W285Y, R320E, and R326E mutation of hAPOBEC3G (SEQ ID NO: 82), or one or more corresponding mutations in another APOBEC deaminase.

Some aspects of this disclosure provide fusion proteins comprising (i) a nuclease-inactive Cas9 domain; and (ii) a nucleic acid editing domain. In some embodiments, a nuclease-inactive Cas9 domain (dCas9), comprises an amino acid sequence that is at least 80%, at least 85%, at least 90%, at least 92%, at least 95%, at least 96%, at least 97%, at least 98%, at least 99%, or at least 99.5% identical to the amino acid sequence of a Cas9 as provided by any one of the Cas9 moieties provided herein, and comprises mutations that inactivate the nuclease activity of Cas9. Mutations that render the nuclease domains of Cas9 inactive are well-known in the art. For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821 (2012); Qi et al., Cell. 28;152 (5): 1173-83 (2013)). In some embodiments, the dCas9 of this disclosure comprises a D10A mutation of the amino acid sequence provided in SEQ ID NO: 107, or a corresponding mutation in any of the amino acid sequences provided herein. In some embodiments, the dCas9 of this disclosure comprises a H840A mutation of the amino acid sequence provided in SEQ ID NO: 107, or a corresponding mutation in any of the amino acid sequences provided herein. In some embodiments, the dCas9 of this disclosure comprises both D10A and H840A mutations of the amino acid sequence provided in SEQ ID NO: 107, or a corresponding mutation in any of the amino acid sequences provided herein. In some embodiments, the Cas9 further comprises a histidine residue at position 840 of the amino acid sequence provided in SEQ ID NO: 107, or a corresponding mutation in any of the amino acid sequences provided herein. The presence of the catalytic residue H840 restores the activity of the Cas9 to cleave the non-edited strand containing a G opposite the targeted C. Restoration of H840 does not result in the cleavage of the target strand containing the C. In some embodiments, the dCas9 comprises an amino acid sequence of SEQ ID NO: 53. It is to be understood that other mutations that inactivate the nuclease domains of Cas9 may also be included in the dCas9 of this disclosure.

Wild type Cas9 corresponding to Cas9 from
Streptococcus pyogenes
(SEQ ID NO: 107)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

The Cas9 or dCas9 domains comprising the mutations disclosed herein, may be a full-length Cas9, or a fragment thereof. In some embodiments, proteins comprising Cas9, or fragments thereof, are referred to as “Cas9 variants.” A Cas9 variant shares homology to Cas9, or a fragment thereof. For example a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9, e.g., a Cas9 comprising the amino acid sequence of SEQ ID NO: 107.

Any of the Cas9 fusion proteins of this disclosure may further comprise a nucleic acid editing domain (e.g., an enzyme that is capable of modifying nucleic acid, such as a deaminase). In some embodiments, the nucleic acid editing domain is a DNA-editing domain. In some embodiments, the nucleic acid editing domain has deaminase activity. In some embodiments, the nucleic acid editing domain comprises or consists of a deaminase or deaminase domain. In some embodiments, the deaminase is a cytidine deaminase. In some embodiments, the deaminase is an apolipoprotein B mRNA-editing complex (APOBEC) family deaminase. In some embodiments, the deaminase is an APOBEC1 family deaminase. In some embodiments, the deaminase is an activation-induced cytidine deaminase (AID). Some nucleic-acid editing domains as well as Cas9 fusion proteins including such domains are described in detail herein. Additional suitable nucleic acid editing domains will be apparent to the skilled artisan based on this disclosure and knowledge in the field.

Some aspects of the disclosure provide a fusion protein comprising a Cas9 domain fused to a nucleic acid editing domain, wherein the nucleic acid editing domain is fused to the N-terminus of the Cas9 domain. In some embodiments, the Cas9 domain and the nucleic acid editing-editing domain are fused via a linker. In some embodiments, the linker comprises a (GGGS), (SEQ ID NO: 109), a (GGGGS), (SEQ ID NO: 110), a (G), (SEQ ID NO: 118), an (EAAAK), (SEQ ID NO: 111), a (GGS), (SEQ ID NO: 112), (SGGS), (SEQ ID NO: 113), an SGSETPGTSESATPES (SEQ ID NO: 114) motif (see, e.g., Guilinger JP, Thompson DB, Liu DR. Fusion of catalytically inactive Cas9 to FokI nuclease improves the specificity of genome modification. Nat. Biotechnol. 2014; 32 (6): 577-82; the entire contents are incorporated herein by reference), or an (XP), motif (SEQ ID NO: 120), or a combination of any of these, wherein n is independently an integer between 1 and 30. In some embodiments, n is independently 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30, or, if more than one linker or more than one linker motif is present, any combination thereof. In some embodiments, the linker comprises a (GGS), motif (SEQ ID NO: 112), wherein n is 1,2, 3, 4, 5, 6, 7, 8, 9, 10, 11, 12, 13, 14 or 15. In some embodiments, the linker comprises a (GGS), motif (SEQ ID NO: 112), wherein n is 1, 3, or 7. In some embodiments, the linker comprises the amino acid sequence SGSETPGTSESATPES (SEQ ID NO: 114). Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65 (10): 1357-69, the entire contents of which are incorporated herein by reference. Additional suitable linker sequences will be apparent to those of skill in the art based on the instant disclosure. In some embodiments, the general architecture of exemplary Cas9 fusion proteins provided herein comprises the structure:
[NH₂]-[nucleic acid editing domain]-[Cas9]-[COOH] or
[NH₂]-[nucleic acid editing domain]-[linker]-[Cas9]-[COOH],
wherein NH₂ is the N-terminus of the fusion protein, and COOH is the C-terminus of the fusion protein.

The fusion proteins of the present disclosure may comprise one or more additional features. For example, in some embodiments, the fusion protein comprises a nuclear localization sequence (NLS). In some embodiments, the NLS of the fusion protein is localized between the nucleic acid editing domain and the Cas9 domain. In some embodiments, the NLS of the fusion protein is localized C-terminal to the Cas9 domain.

In some embodiments, the nucleic acid editing domain is a deaminase. For example, in some embodiments, the general architecture of exemplary Cas9 fusion proteins with a deaminase domain comprises the structure:
[NH₂]-[NLS]-[deaminase]-[Cas9]-[COOH],
[NH₂]-[Cas9]-[deaminase]-[COOH],
[NH₂]-[deaminase]-[Cas9]-[COOH], or
[NH₂]-[deaminase]-[Cas9]-[NLS]-[COOH]
wherein NLS is a nuclear localization sequence, NH₂ is the N-terminus of the fusion protein, and COOH is the C-terminus of the fusion protein. Nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, a NLS comprises the amino acid sequence PKKKRKV (SEQ ID NO: 115) or MDSLLMNRRKFLYQFKNVRWAKGRRETYLC (SEQ ID NO: 116). In some embodiments, a linker is inserted between the Cas9 and the deaminase. In some embodiments, the NLS is located C-terminal of the Cas9 domain. In some embodiments, the NLS is located N-terminal of the Cas9 domain. In some embodiments, the NLS is located between the deaminase and the Cas9 domain. In some embodiments, the NLS is located N-terminal of the deaminase domain. In some embodiments, the NLS is located C-terminal of the deaminase domain.

One exemplary suitable type of nucleic acid editing domain is a cytidine deaminase, for example, of the APOBEC family. The apolipoprotein B mRNA-editing complex (APOBEC) family of cytidine deaminase enzymes encompasses eleven proteins that serve to initiate mutagenesis in a controlled and beneficial manner.²⁹ One family member, activation-induced cytidine deaminase (AID), is responsible for the maturation of antibodies by converting cytosines in ssDNA to uracils in a transcription-dependent, strand-biased fashion.³⁰ The apolipoprotein B editing complex 3 (APOBEC3) enzyme provides protection to human cells against a certain HIV-1 strain via the deamination of cytosines in reverse-transcribed viral ssDNA. These proteins all require a Zn²⁺-coordinating motif (His-X-Glu-X₂₃₋₂₆-Pro-Cys-X₂₋₄-Cys; SEQ ID NO: 117) and bound water molecule for catalytic activity. The Glu residue acts to activate the water molecule to a zinc hydroxide for nucleophilic attack in the deamination reaction. Each family member preferentially deaminates at its own particular “hotspot”, ranging from WRC (W is A or T, R is A or G) for hAID, to TTC for hAPOBEC3F.³² A recent crystal structure of the catalytic domain of APOBEC3G revealed a secondary structure comprised of a five-stranded B-sheet core flanked by six a-helices, which is believed to be conserved across the entire family.³³ The active center loops have been shown to be responsible for both ssDNA binding and in determining “hotspot” identity.³⁴ Overexpression of these enzymes has been linked to genomic instability and cancer, thus highlighting the importance of sequence-specific targeting.

Some aspects of this disclosure relate to the recognition that the activity of cytidine deaminase enzymes such as APOBEC enzymes can be directed to a specific site in genomic DNA. Without wishing to be bound by any particular theory, advantages of using Cas9 as a recognition agent include (1) the sequence specificity of Cas9 can be easily altered by simply changing the sgRNA sequence; and (2) Cas9 binds to its target sequence by denaturing the dsDNA, resulting in a stretch of DNA that is single-stranded and therefore a viable substrate for the deaminase. It should be understood that other catalytic domains, or catalytic domains from other deaminases, can also be used to generate fusion proteins with Cas9, and that the disclosure is not limited in this regard.

Some aspects of this disclosure are based on the recognition that Cas9: deaminase fusion proteins can efficiently deaminate nucleotides. In view of the results provided herein regarding the nucleotides that can be targeted by Cas9: deaminase fusion proteins, a person of skill in the art will be able to design suitable guide RNAs to target the fusion proteins to a target sequence that comprises a nucleotide to be deaminated.

In some embodiments, the deaminase domain and the Cas9 domain are fused to each other via a linker. Various linker lengths and flexibilities between the deaminase domain (e.g., AID) and the Cas9 domain can be employed (e.g., ranging from very flexible linkers of the form (GGGGS), (SEQ ID NO: 110), (GGS)ₙ, (SEQ ID NO: 112) and (G)ₙ (SEQ ID NO: 118) to more rigid linkers of the form (EAAAK)ₙ, (SEQ ID NO: 111), (SGGS)ₙ (SEQ ID NO: 113), SGSETPGTSESATPES (SEQ ID NO: 114) (see, e.g., Guilinger JP, Thompson DB, Liu DR. Fusion of catalytically inactive Cas9 to FokI nuclease improves the specificity of genome modification. Nat. Biotechnol. 2014; 32 (6): 577-82; the entire contents are incorporated herein by reference) and (XP), (SEQ ID NO: 120)) 36 in order to achieve the optimal length for deaminase activity for the specific application. In some embodiments, the linker comprises a (GGS), motif (SEQ ID NO: 112), wherein n is 1, 3, or 7. In some embodiments, the linker comprises a (an SGSETPGTSESATPES (SEQ ID NO: 114) motif.

Some exemplary suitable nucleic-acid editing domains, e.g., deaminases and deaminase domains, that can be fused to Cas9 domains according to aspects of this disclosure are provided below. It should be understood that, in some embodiments, the active domain of the respective sequence can be used, e.g., the domain without a localizing signal (nuclear localization sequence, without nuclear export signal, cytoplasmic localizing signal).

### Adenosine Deaminases

Some aspects of the disclosure provide adenosine deaminases. In some embodiments, the adenosine deaminases provided herein are capable of deaminating adenosine. In some embodiments, the adenosine deaminases provided herein are capable of deaminating adenosine in a deoxyadenosine residue of DNA. The adenosine deaminase may be derived from any suitable organism (e.g., E. coli). In some embodiments, the adenosine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase (e.g., having homology to ccTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 15, or to any of the adenosine deaminases provided herein. It should be appreciated that adenosine deaminases provided herein may include one or more mutations (e.g., any of the mutations provided herein). The disclosure provides any deaminase domains with a certain percent identiy plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NO: 15 or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NO: 15 or any of the adenosine deaminases provided herein.

In some embodiments, the adenosine deaminase comprises a D108X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises a D108G, D108N, D108V, D108A, or D108Y mutation in SEQ ID NO: 15, or a corresponding mutation in another adenosine deaminase. It should be appreciated, however, that additional deaminases may similarly be aligned to identify homologous amino acid residues that can be mutated as provided herein.

In some embodiments, the adenosine deaminse comprises an A106X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an A106V mutation in SEQ ID NO: 15, or a corresponding mutation in another adenosine deaminase.

In some embodiments, the adenosine deaminase comprises a E155X mutation in SEQ ID NO: 15, or a corresponding mutation in another adenosine deaminase, where the presence of X indicates any amino acid other than the corresponding amino acid in the wild-type adenosine deaminase. In some embodiments, the adenosine deaminase comprises a E155D, E155G, or E155V mutation in SEQ ID NO: 15, or a corresponding mutation in another adenosine deaminase.

In some embodiments, the adenosine deaminase comprises a D147X mutation in SEQ ID NO: 15, or a corresponding mutation in another adenosine deaminase, where the presence of X indicates any amino acid other than the corresponding amino acid in the wild-type adenosine deaminase. In some embodiments, the adenosine deaminase comprises a D147Y, mutation in SEQ ID NO: 15, or a corresponding mutation in another adenosine deaminase.

It should be appreciated that any of the mutations provided herein (e.g., based on the ecTadA amino acid sequence of SEQ ID NO: 15) may be introduced into other adenosine deaminases, such as S. aureus TadA (saTadA), or other adenosine deaminases (e.g., bacterial adenosine deaminases). It would be apparent to the skilled artisan how to identify amino acid residues from other adenosine deaminases that are homologous to the mutated residues in ecTadA. Thus, any of the mutations identified in ecTadA may be made in other adenosine deaminases that have homologous amino acid residues. It should also be appreciated that any of the mutations provided herein may be made individually or in any combination in ecTadA or another adenosine deaminase. For example, an adenosine deaminase may contain a D108N, a A106V, a E155V, and/or a D147Y mutation in ecTadA SEQ ID NO: 15, or a corresponding mutation in another adenosine deaminase. In some embodiments, an adenosine deaminase comprises the following group of mutations (groups of mutations are separated by a “;”) in ecTadA SEQ ID NO: 15, or corresponding mutations in another adenosine deaminase:

D108N and A106V; D108N and E155V; D108N and D147Y; A106V and E155V; A106V and D147Y; E155V and D147Y; D108N, A106V, and E55V; D108N, A106V, and D147Y; D108N, E55V, and D147Y; A106V, E55V, and D147Y; and D108N, A106V, E55V, and D147Y. It should be appreciated, however, that any combination of corresponding mutations provided herein may be made in an adenosine deaminase (e.g., ccTadA).

In some embodiments, the adenosine deaminase comprises one or more of a H8X, T17X, L18X, W23X, L34X, W45X, R51X, A56X, E59X, E85X, M94X, 195X, V102X, F104X, A106X, R107X, D108X, K110X, M118X, N127X, A138X, F149X, M151X, R153X, Q154X, 1156X, and/or K157X mutation in SEQ ID NO: 15, or one or more corresponding mutations in another adenosine deaminase, where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, 1951, V102A, F104L, A106V, R107C, or R107H, or R107P, D108G, or D108N, or D108V, or D108A, or D108Y, K110I, M118K, N127S, A138V, F149Y, M151V, R153C, Q154L, 1156D, and/or K157R mutation in SEQ ID NO: 15, or one or more corresponding mutations in another adenosine deaminase.

In some embodiments, the adenosine deaminase comprises one or more of a H8X, D108X, and/or N127X mutation in SEQ ID NO: 15, or one or more corresponding mutations in another adenosine deaminase, where X indicates the presence of any amino acid. In some embodiments, the adenosine deaminase comprises one or more of a H8Y, D108N, and/or N127S mutation in SEQ ID NO: 15, or one or more corresponding mutations in another adenosine deaminase.

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in SEQ ID NO: 15, or one or more corresponding mutations in another adenosine deaminase, where X indicates the presence of any amino acid other than the corresponding amino acid in the wild-type adenosine deaminase. In some embodiments, the adenosine deaminase comprises one or more of H8Y, R26W, M61I, L68Q, M70V, A106T, D108N, A109T, N127S, D147Y, R152C, Q154H or Q154R, E155G or E155V or E155D, K161Q, Q163H, and/or T166P mutation in SEQ ID NO: 15, or one or more corresponding mutations in another adenosine deaminase.

In some embodiments, the adenosine deaminase comprises one, two, three, four, five, or six mutations selected from the group consisting of H8X, D108X, N127X, D147X, R152X, and Q154X in SEQ ID NO: 15, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in SEQ ID NO: 15, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in SEQ ID NO: 15, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of H8X, A106X, D108X, N127X, E155X, and K161X in SEQ ID NO: 15, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R126X, L68X, D108X, N127X, D147X, and E155X in SEQ ID NO: 15, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, A109X, N127X, and E155X in SEQ ID NO: 15, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in SEQ ID NO: 15, or a corresponding mutation or mutations in another adenosine deaminase. In some embodiments, the adenosine deaminase comprises one, two, three, four, five, six, seven, or eight mutations selected from the group consisting of H8Y, M61I, M70V, D108N, N127S, Q154R, E155G and Q163H in SEQ ID NO: 15, or a corresponding mutation or mutations in another adenosine deaminase. In some embodiments, the adenosine deaminase comprises one, two, three, four, or five, mutations selected from the group consisting of H8Y, D108N, N127S, E155V, and T166P in SEQ ID NO: 15, or a corresponding mutation or mutations in another adenosine deaminase. In some embodiments, the adenosine deaminase comprises one, two, three, four, five, or six mutations selected from the group consisting of H8Y, A106T, D108N, N127S, E155D, and K161Q in SEQ ID NO: 15, or a corresponding mutation or mutations in another adenosine deaminase. In some embodiments, the adenosine deaminase comprises one, two, three, four, five, six, seven, or eight mutations selected from the group consisting of H8Y, R126W, L68Q, D108N, N127S, D147Y, and E155V in SEQ ID NO: 15, or a corresponding mutation or mutations in another adenosine deaminase. In some embodiments, the adenosine deaminase comprises one, two, three, four, or five, mutations selected from the group consisting of H8Y, D108N, A109T, N127S, and E155G in SEQ ID NO: 15, or a corresponding mutation or mutations in another adenosine deaminase.

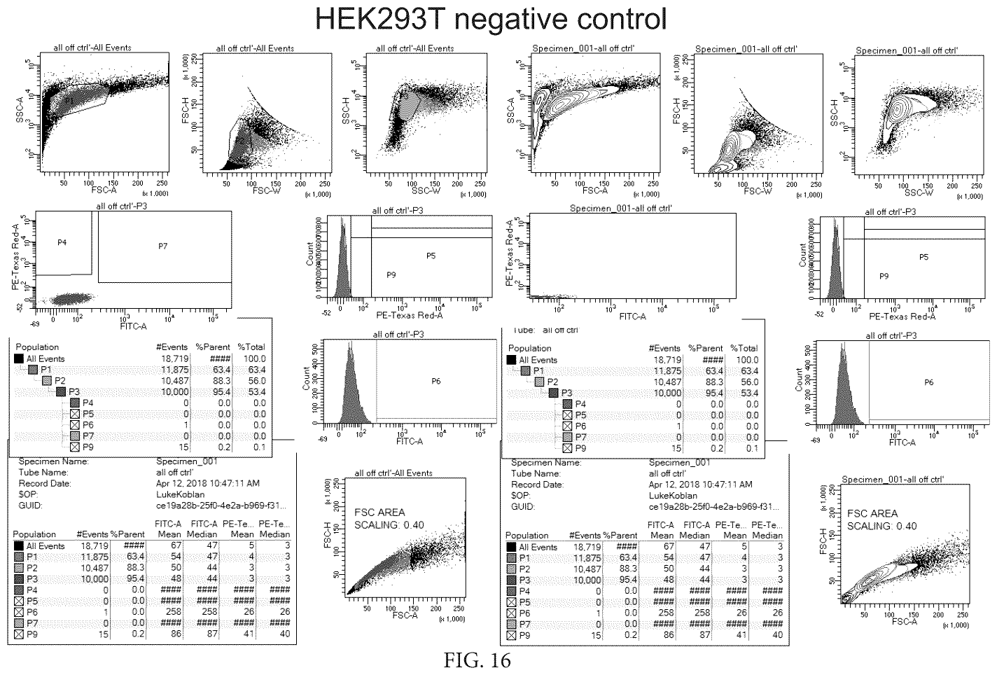

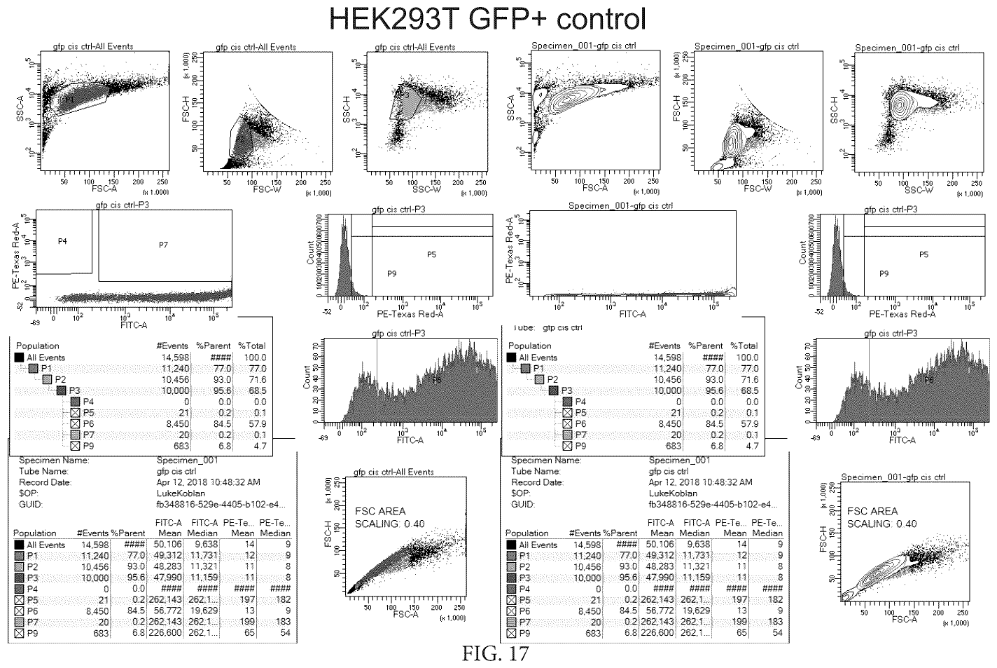

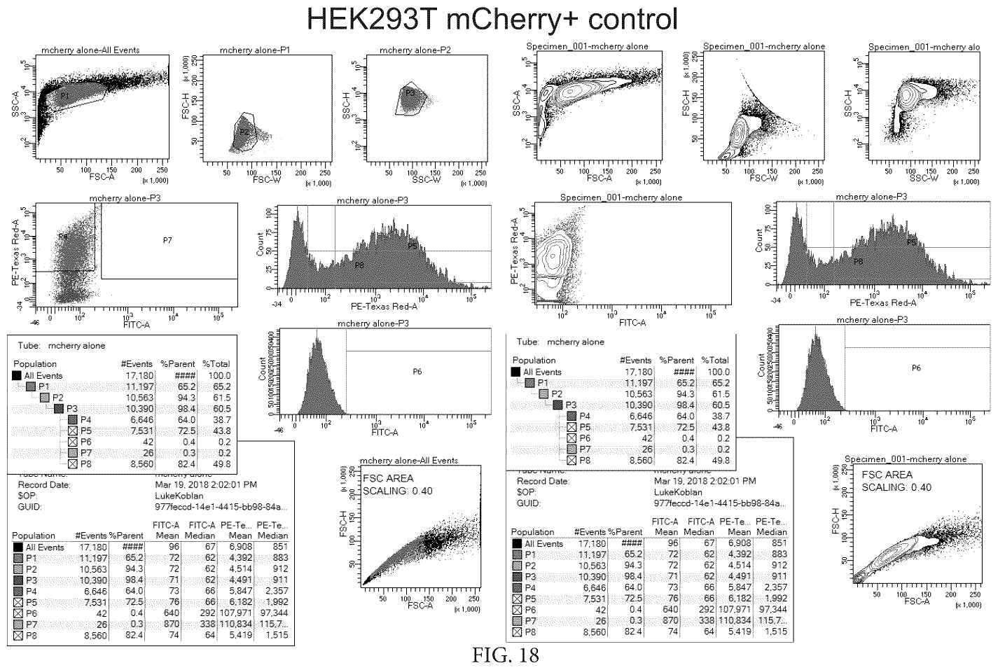

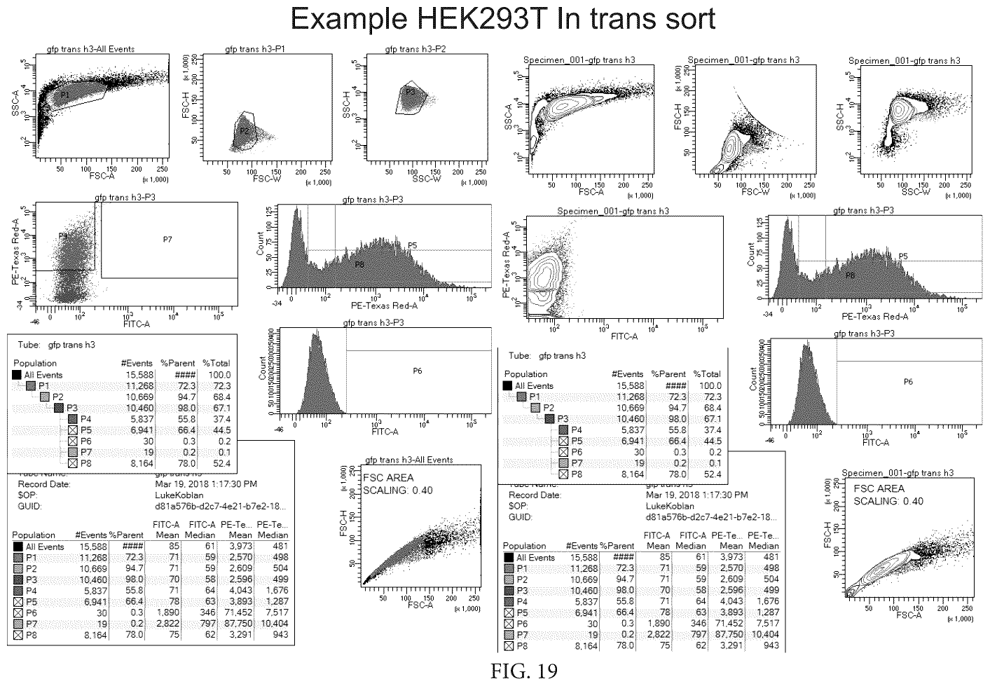

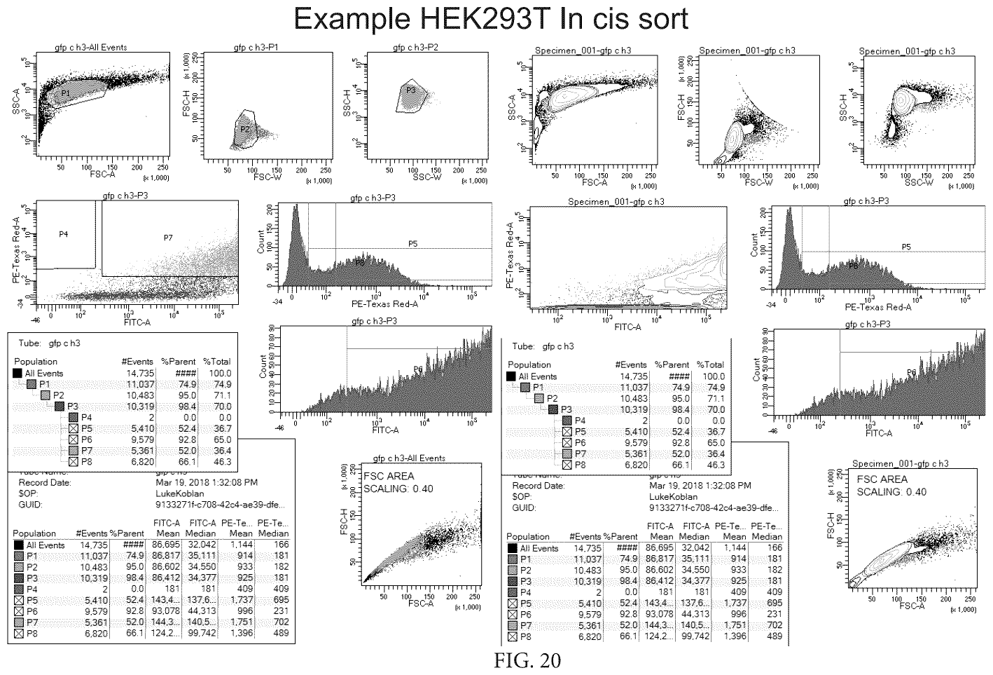

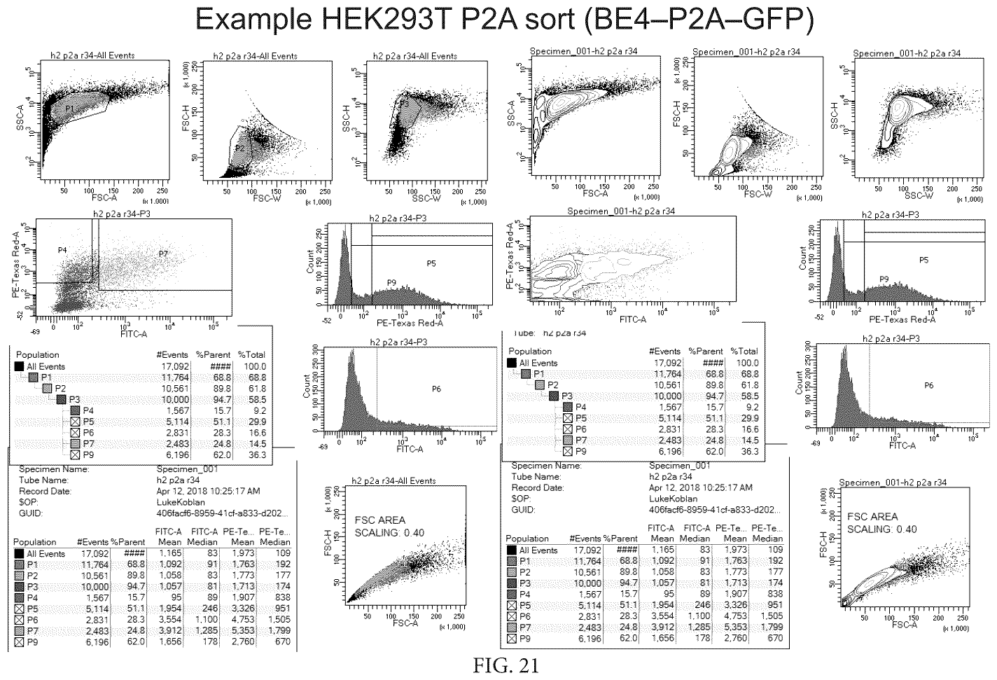

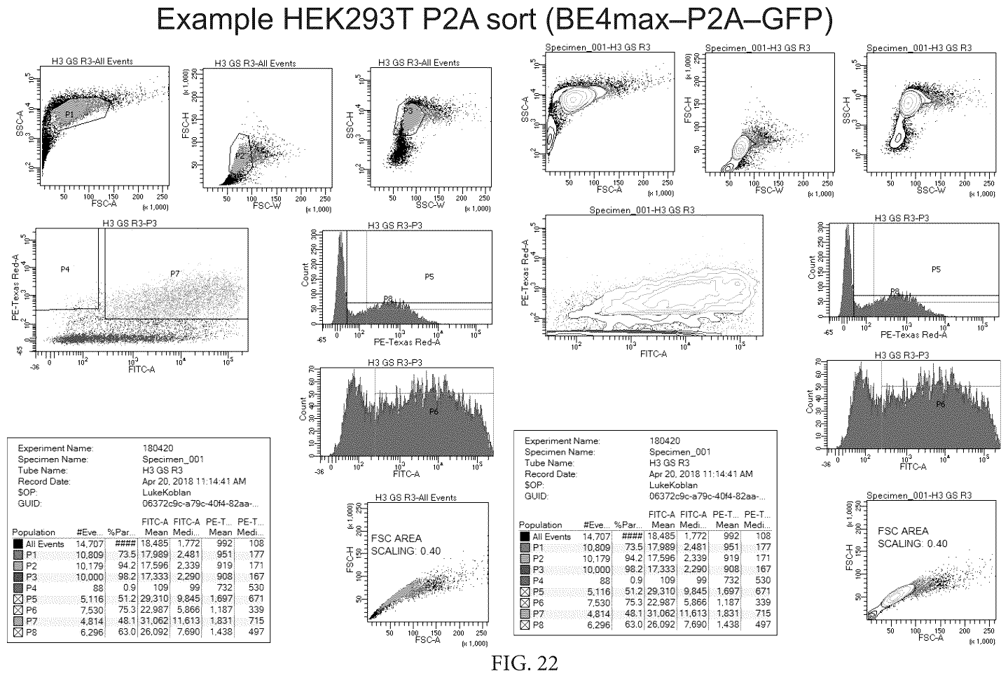

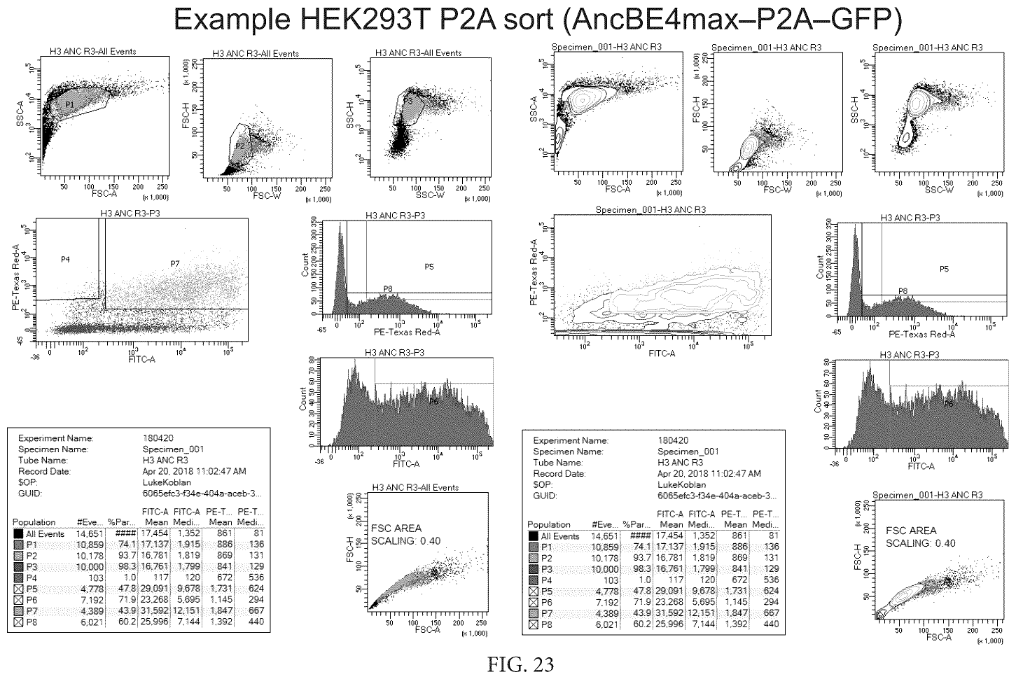

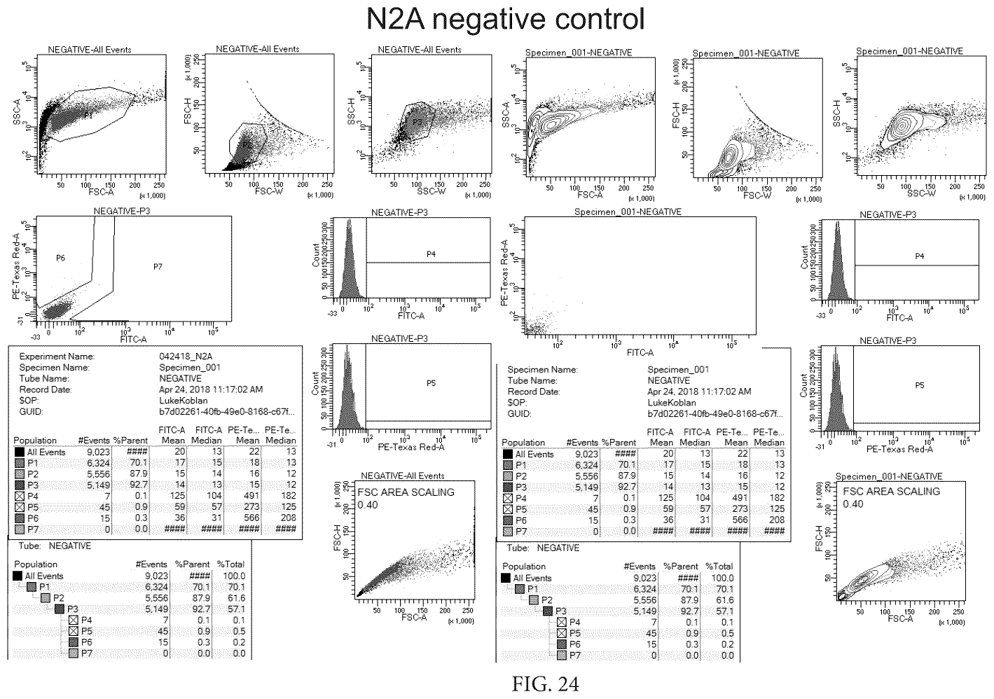

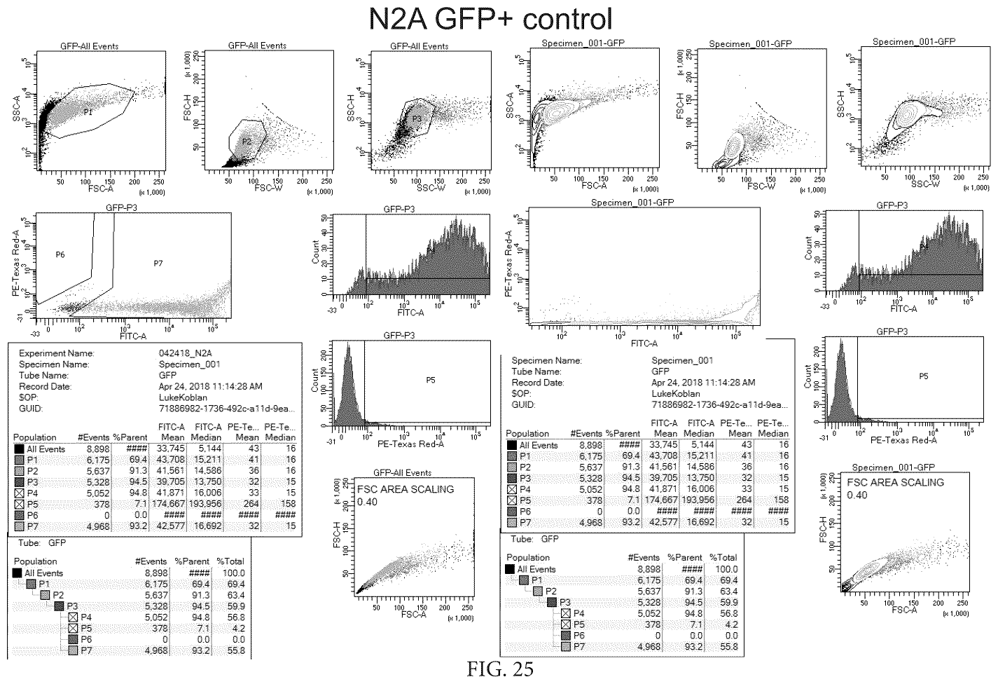

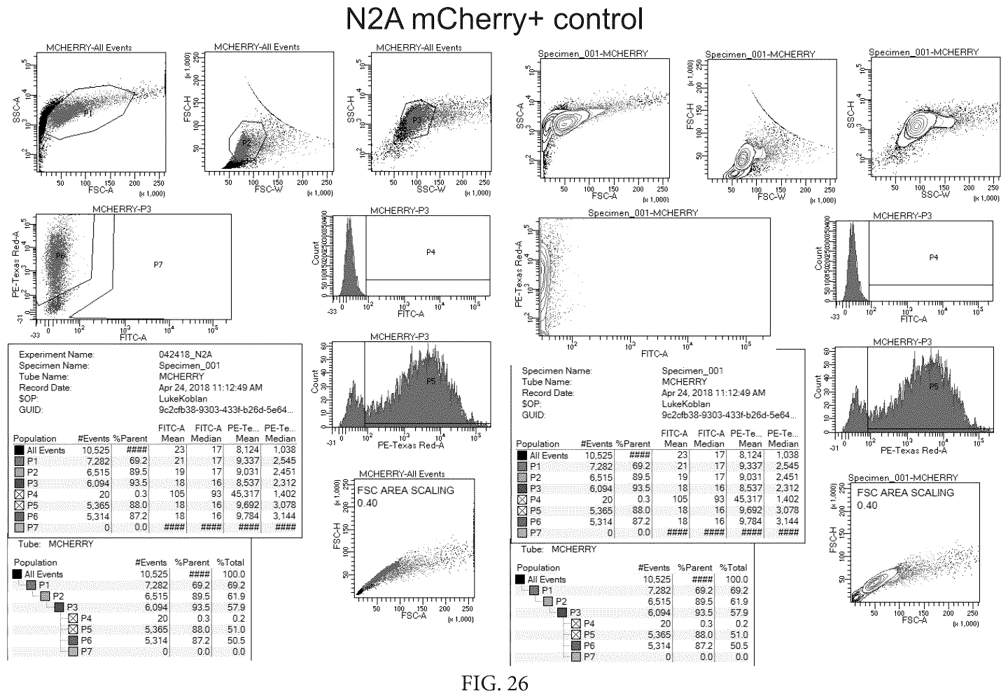

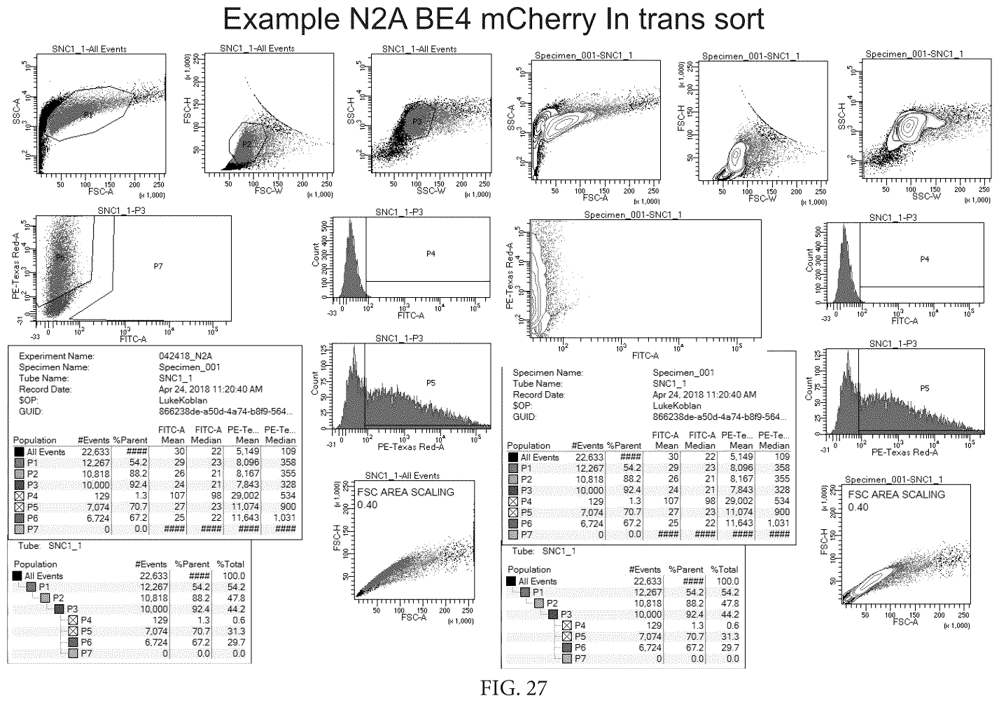

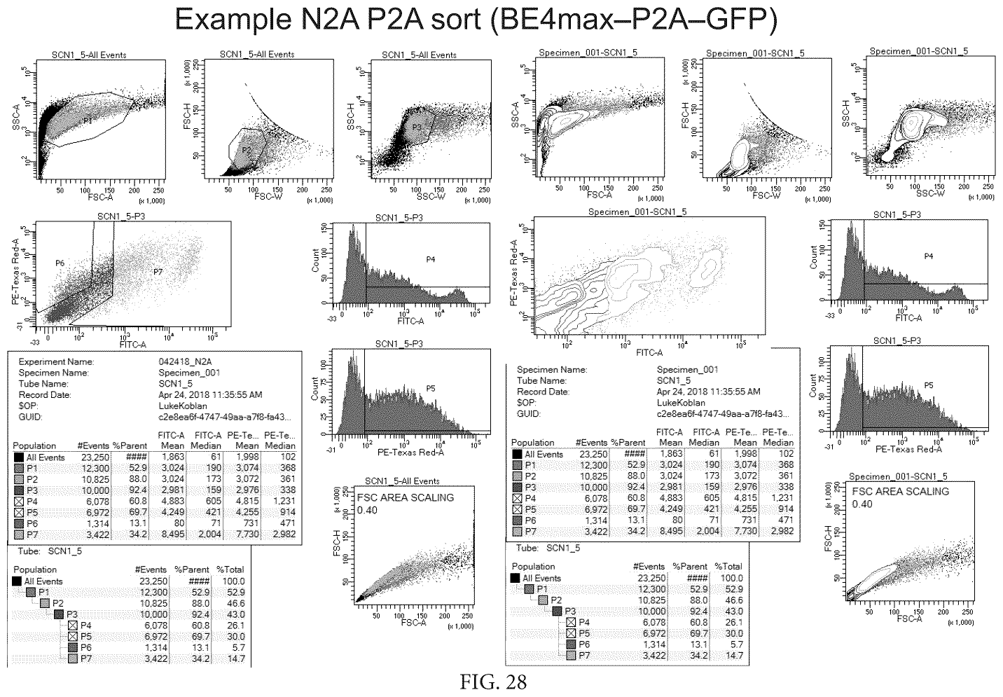

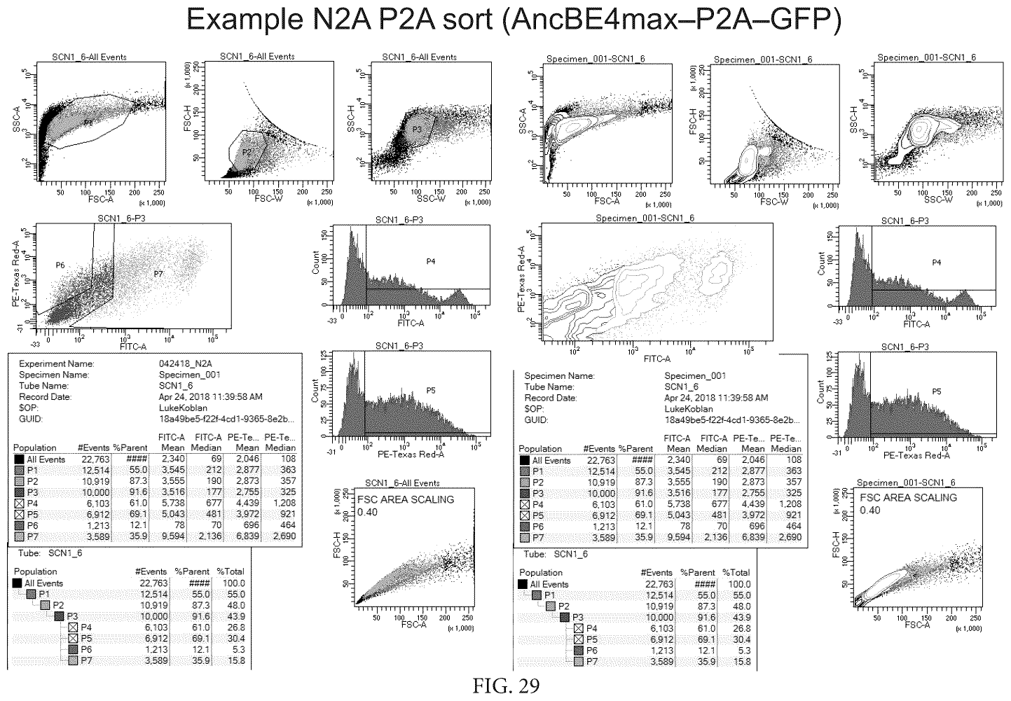

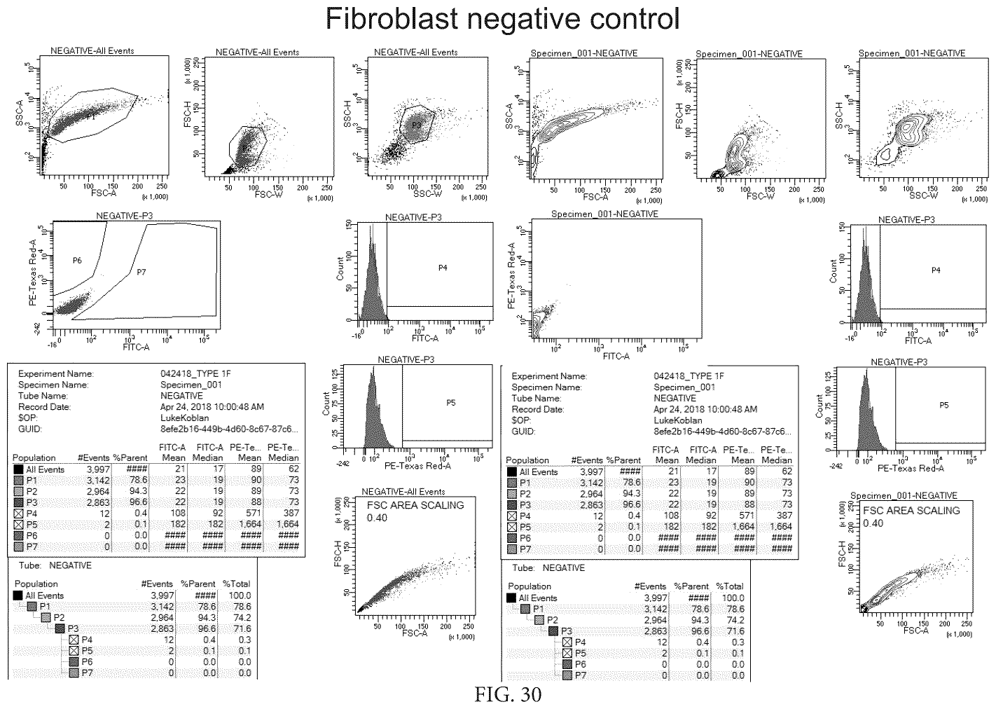

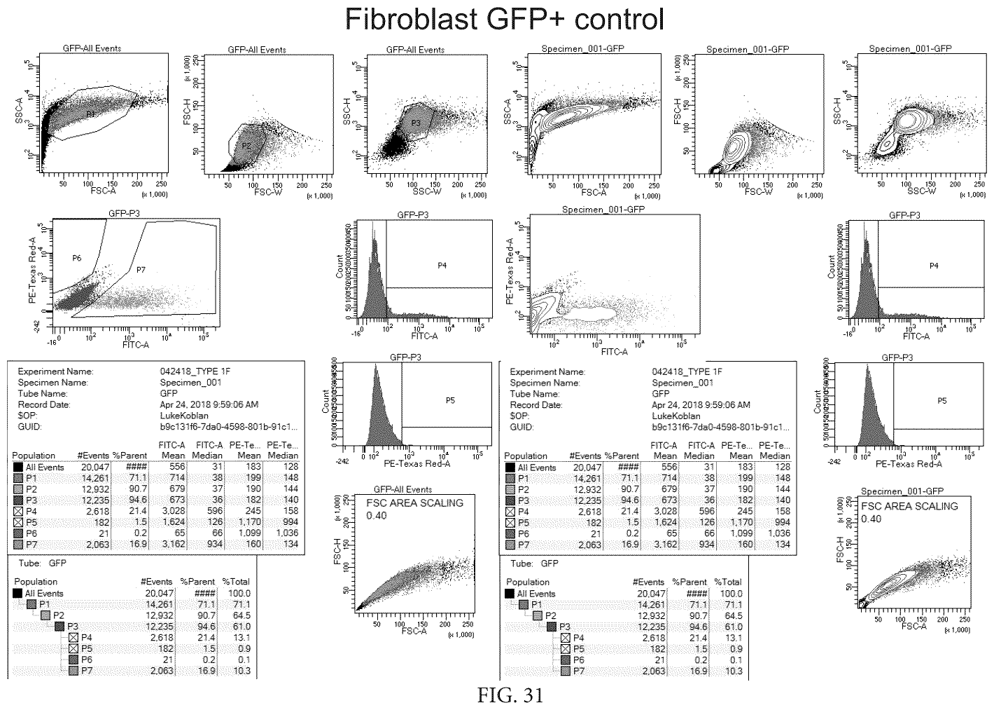

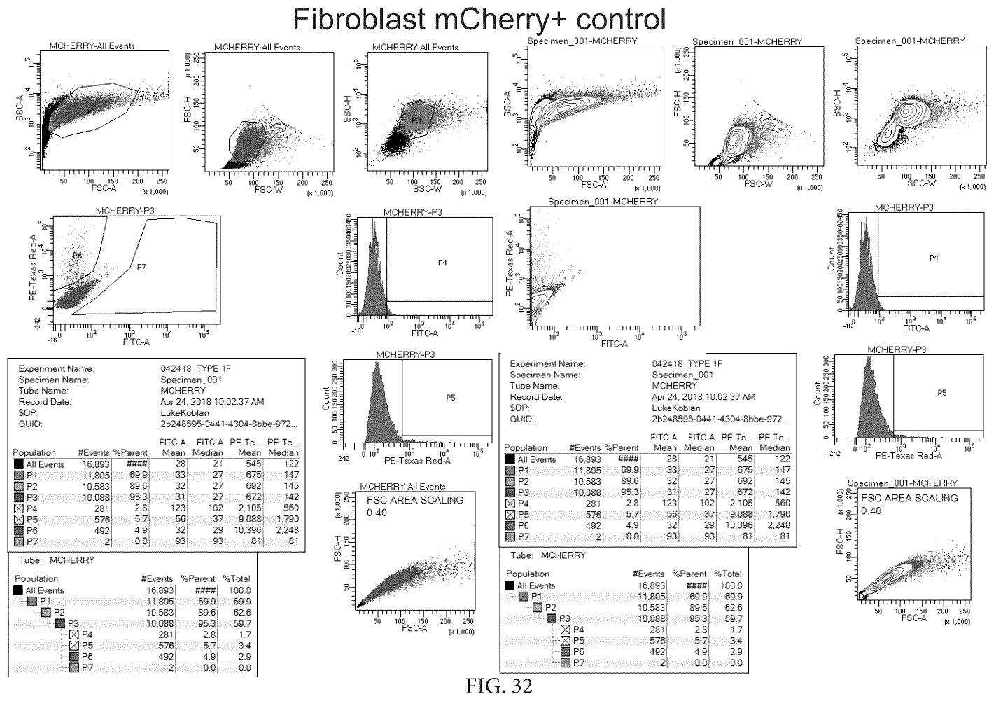

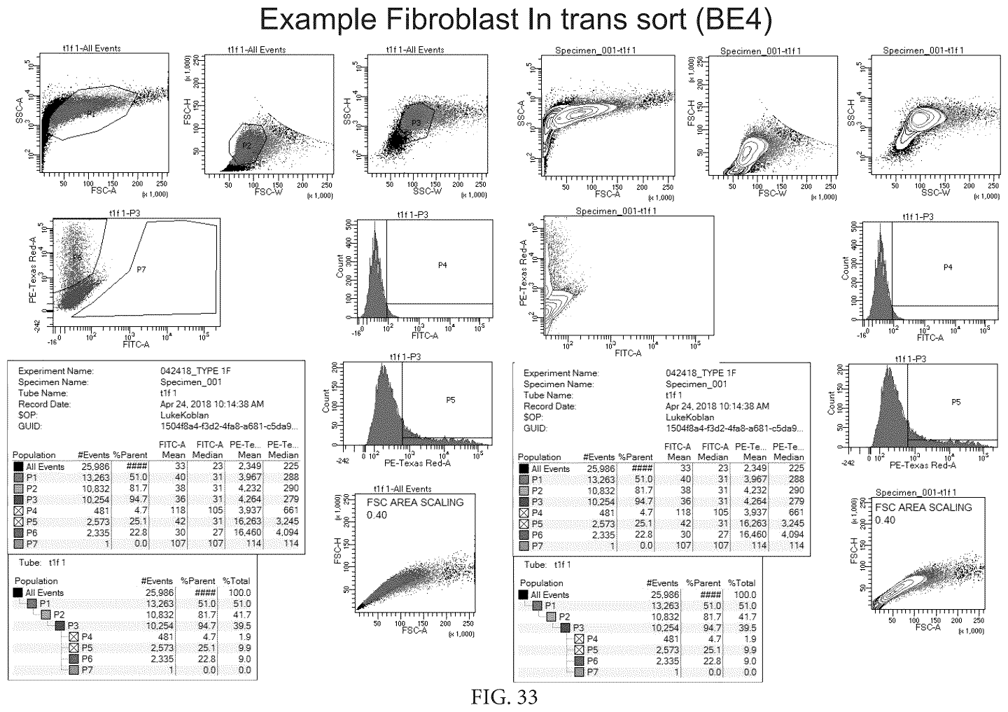

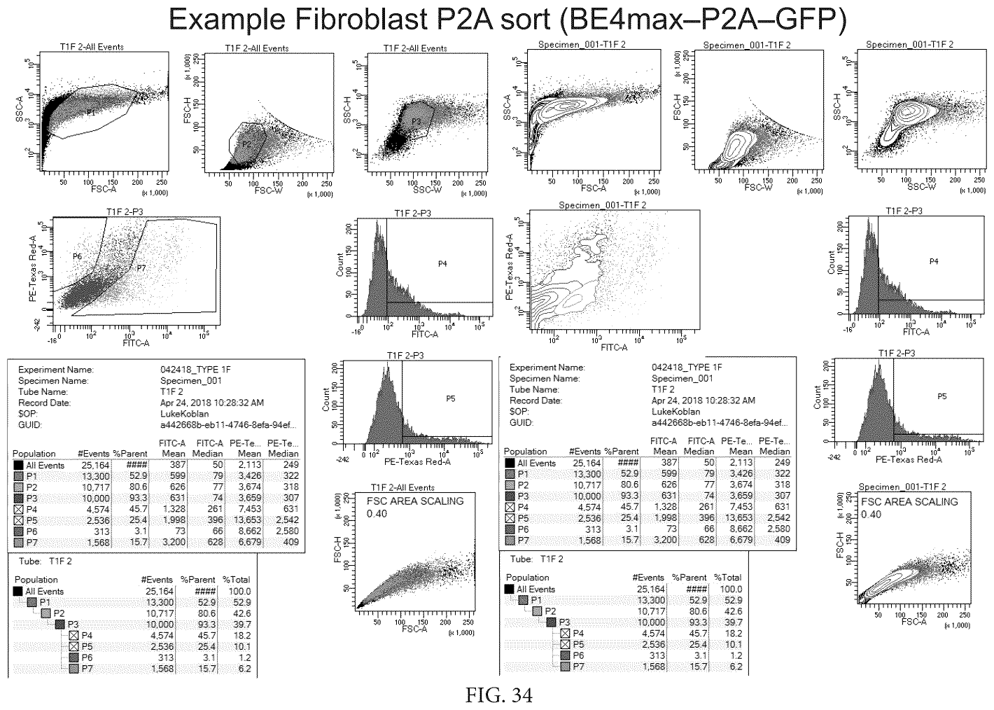

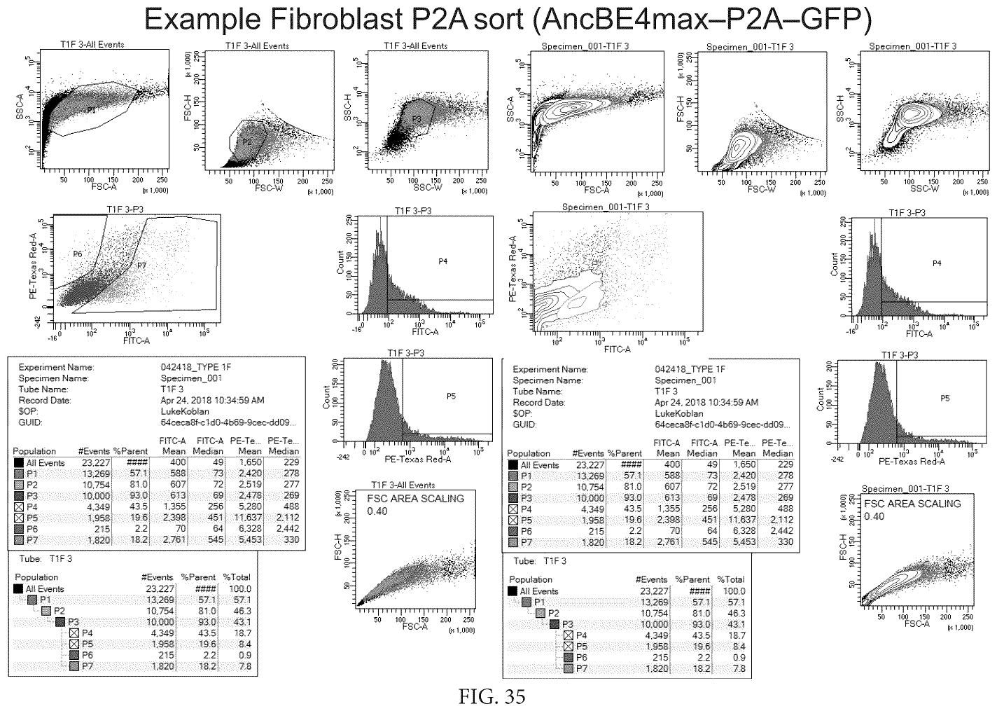

In some embodiments, the adenosine deaminase comprises one or more of the mutations provided in FIG. 16 corresponding to SEQ ID NO: 15, or one or more corresponding mutations in another adenosine deaminase. In some embodiments, the adenosine deaminase comprises a D108N, D108G, or D108V mutation in SEQ ID NO: 15, or corresponding mutations in another adenosine deaminase. In some embodiments, the adenosine deaminase comprises a A106V and D108N mutation in SEQ ID NO: 15, or corresponding mutations in another adenosine deaminase. In some embodiments, the adenosine deaminase comprises R107C and D108N mutations in SEQ ID NO: 15, or corresponding mutations in another adenosine deaminase. In some embodiments, the adenosine deaminase comprises a H8Y, D108N, N127S, D147Y, and Q154H mutation in SEQ ID NO: 15, or corresponding mutations in another adenosine deaminase. In some embodiments, the adenosine deaminase comprises a H8Y, R24W, D108N, N127S, D147Y, and E155V mutation in SEQ ID NO: 15, or corresponding mutations in another adenosine deaminase. In some embodiments, the adenosine deaminase comprises a D108N, D147Y, and E155V mutation in SEQ ID NO: 15, or corresponding mutations in another adenosine deaminase. In some embodiments, the adenosine deaminase comprises a H8Y, D108N, and S127S mutation in SEQ ID NO: 15, or corresponding mutations in another adenosine deaminase. In some embodiments, the adenosine deaminase comprises a A106V, D108N, D147Y and E155V mutation in SEQ ID NO: 15, or corresponding mutations in another adenosine deaminase.

In some embodiments, the adenosine deaminase comprises one or more of a, S2X, H8X, 149X, L84X, H123X, N127X, 1156X and/or K160X mutation in SEQ ID NO: 15, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, 149F, L84F, H123Y, N127S, 1156F and/or K160S mutation in SEQ ID NO: 15, or one or more corresponding mutations in another adenosine deaminase.

In some embodiments, the adenosine deaminse comprises an L84X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an L84F mutation in SEQ ID NO: 15, or a corresponding mutation in another adenosine deaminase.

In some embodiments, the adenosine deaminse comprises an H123X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an H123Y mutation in SEQ ID NO: 15, or a corresponding mutation in another adenosine deaminase.

In some embodiments, the adenosine deaminse comprises an 1157X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an 1157F mutation in SEQ ID NO: 15, or a corresponding mutation in another adenosine deaminase.

In some embodiments, the adenosine deaminase comprises one, two, three, four, five, six, or seven mutations selected from the group consisting of L84X, A106X, D108X, H123X, D147X, E155X, and 1156X in SEQ ID NO: 15, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, five, or six mutations selected from the group consisting of S2X, 149X, A106X, D108X, D147X, and E155X in SEQ ID NO: 15, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five, mutations selected from the group consisting of H8X, A106X, D108X, N127X, and K160X in SEQ ID NO: 15, or a corresponding mutation or mutations in another adenosine deaminase, where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and 1156F in SEQ ID NO: 15, or a corresponding mutation or mutations in another adenosine deaminase. In some embodiments, the adenosine deaminase comprises one, two, three, four, five, or six mutations selected from the group consisting of S2A, 149F, A106V, D108N, D147Y, and E155V in SEQ ID NO: 15, or a corresponding mutation or mutations in another adenosine deaminase. In some embodiments, the adenosine deaminase comprises one, two, three, four, or five, mutations selected from the group consisting of H8Y, A106T, D108N, N127S, and K160S in SEQ ID NO: 15, or a corresponding mutation or mutations in another adenosine deaminase.

In some embodiments, the adenosine deaminase comprises one or more of a, E25X, R26X, R107X, A142X, and/or A143X mutation in SEQ ID NO: 15, or one or more corresponding mutations in another adenosine deaminase, where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R07K, R107A, R107N, R107W, R107H, R107S, A142N, A142D, A142G, A143D, A143G, A143E, A143L, A143W, A143M, A143S, A143Q and/or A143R mutation in SEQ ID NO: 15, or one or more corresponding mutations in another adenosine deaminase.

In some embodiments, the adenosine deaminse comprises an E25X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an E25M, E25D, E25A, E25R, E25V, E25S, or E25Y mutation in SEQ ID NO: 15, or a corresponding mutation in another adenosine deaminase.

In some embodiments, the adenosine deaminse comprises an R26X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an, R26G, R26N, R26Q, R26C, R26L, or R26K mutation in SEQ ID NO: 15, or a corresponding mutation in another adenosine deaminase.

In some embodiments, the adenosine deaminse comprises an R107X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an R107P, R07K, R107A, R107N, R107W, R107H, or R107S mutation in SEQ ID NO: 15, or a corresponding mutation in another adenosine deaminase.

In some embodiments, the adenosine deaminse comprises an A142X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an A142N, A142D, A142G, mutation in SEQ ID NO: 15, or a corresponding mutation in another adenosine deaminase.

In some embodiments, the adenosine deaminse comprises an A143X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an A143D, A143G, A143E, A143L, A143W, A143M, A143S, A143Q and/or A143R mutation in SEQ ID NO: 15, or a corresponding mutation in another adenosine deaminase.

In some embodiments, the adenosine deaminase comprises one or more of a, H36X, N37X, P48X, 149X, R51X, M70X, N72X, D77X, E134X, S146X, Q154X, K157X, and/or K161X mutation in SEQ ID NO: 15, or one or more corresponding mutations in another adenosine deaminase, where the presence of X indicates any amino acid other than the corresponding amino acid in the wild-type adenosine deaminase. In some embodiments, the adenosine deaminase comprises one or more of H36L, N37T, N37S, P48T, P48L, 149V, R51H, R51L, M70L, N72S, D77G, E134G, S146R, S146C, Q154H, K157N, and/or K16IT mutation in SEQ ID NO: 15, or one or more corresponding mutations in another adenosine deaminase.

In some embodiments, the adenosine deaminse comprises an H36X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an H36L mutation in SEQ ID NO: 15, or a corresponding mutation in another adenosine deaminase.

In some embodiments, the adenosine deaminse comprises an N37X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an N37T, or N37S mutation in SEQ ID NO: 15, or a corresponding mutation in another adenosine deaminase.

In some embodiments, the adenosine deaminse comprises an P48X mutation in ccTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an P48T, or P48L mutation in SEQ ID NO: 15, or a corresponding mutation in another adenosine deaminase.

In some embodiments, the adenosine deaminse comprises an R51X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an R51H, or R51L mutation in SEQ ID NO: 15, or a corresponding mutation in another adenosine deaminase.

In some embodiments, the adenosine deaminse comprises an S146X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises an S146R, or S146C mutation in SEQ ID NO: 15, or a corresponding mutation in another adenosine deaminase.

In some embodiments, the adenosine deaminse comprises an K157X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises a K157N mutation in SEQ ID NO: 15, or a corresponding mutation in another adenosine deaminase.

In some embodiments, the adenosine deaminse comprises an P48X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises a P48S, P48T, or P48A mutation in SEQ ID NO: 15, or a corresponding mutation in another adenosine deaminase.

In some embodiments, the adenosine deaminse comprises an A142X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises a A142N mutation in SEQ ID NO: 15, or a corresponding mutation in another adenosine deaminase.

In some embodiments, the adenosine deaminse comprises an W23X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises a W23R, or W23L mutation in SEQ ID NO: 15, or a corresponding mutation in another adenosine deaminase.

In some embodiments, the adenosine deaminse comprises an R152X mutation in ecTadA SEQ ID NO: 15, or a corresponding mutation in another adenosine deaminase, where X indicates any amino acid other than the corresponding amino acid in the wild-type adenosine deaminase. In some embodiments, the adenosine deaminase comprises a R152P, or R52H mutation in SEQ ID NO: 15, or a corresponding mutation in another adenosine deaminase.

In some embodiments, the adenosine deaminase comprises the combination of mutations of any of the adenosine deaminases (e.g., ecTadA adenosine deaminases) described herein. For example, the adenosine deaminase may comprise the mutations H36L, R51L, L84F, A106V, D108N, H123Y, S146C, D147Y, E155V, 1156F, and K157N (relative to SEQ ID NO: 15) of clone pNMG-477. In some embodiments, the adenosine deaminase comprises the following combination of mutations relative to SEQ ID NO: 15, where each mutation of a combination is separated by a “_” and each combination of mutations is between parentheses: (A106V_D108N), (R107C_D108N), (H8Y_D108N_S127S_D147Y_Q154H), (H8Y_R24W_D108N_N127S_D147Y_E155V), (D108N_D147Y_E155V), (H8Y_D108N_S127S), (H8Y_D108N_N127S_D147Y_Q154H), (A106V_D108N_D147Y_E155V), (D108Q_D147Y_E155V), (D108M_D147Y_E155V), (D108L_D147Y_E155V), (D108K_D147Y_E155V), (D108I_D147Y_E155V), (D108F_D147Y_E155V), (A106V_D108N_D147Y), (A106V_D108M_D147Y_E155V), (E59A_A106V_D108N_D147Y_E155V), (E59A cat dead_A106V_D108N_D147Y_E155V), (L84F_A106V_D108N_H123Y_D147Y_E155V_1156Y), (L84F_A106V_D108N_H123Y_D147Y_E155V_1156F), (D103A_D014N), (G22P_D103A_D104N), (G22P_D103A_D104N_S138A), (D103A_D104N_S138A), (R26G_L84F_A106V_R107H_D108N_H123Y_A142N_A143D_D147Y_E155V_1156F), (E25G_R26G_L84F_A106V_R107H_D108N_H123Y_A142N_A143D_D147Y_E155V_115 6F), (E25D_R26G_L84F_A106V_R107K_D108N_H123Y_A142N_A143G_D147Y_E155V _1156), (R26Q_L84F_A106V_D108N_H123Y_A142N_D147Y_E155V_1156F), (E25M_R26G_L84F_A106V_R107P_D108N_H123Y_A142N_A143D_D147Y_E155V_115 6F), (R26C_L84F_A106V_R107H_D108N_H123Y_A142N_D147Y_E155V_1156F), (L84F_A106V_D108N_H123Y_A142N_A143L_D147Y_E155V_1156F), (R26G_L84F_A106V_D108N_H123Y_A142N_D147Y_E155V_1156F), (E25A_R26G_L84F_A106V_R107N_D108N_H123Y_A142N_A143E_D147Y_E155V_115 6F), (R26G_L84F_A106V_R107H_D108N_H123Y_A142N_A143D_D147Y_E155V_1156F), (A106V_D108N_A142N_D147Y_E155V), (R26G_A106V_D108N_A142N_D147Y_E155V), (E25D_R26G_A106V_R107K_D108N_A142N_A143G_D147Y_E155V), (R26G_A106V_D108N_R107H_A142N_A143D_D147Y_E155V), (E25D_R26G_A106V_D108N_A142N_D147Y_E155V), (A106V_R107K_D108N_A142N_D147Y_E155V), (A106V_D108N_A142N_A143G_D147Y_E155V), (A106V_D108N_A142N_A143L_D147Y_E155V), (H36L_R51L_L84F_A106V_D108N_H123Y_S146C_D147Y_E155V_1156F_K157N), (N37T_P48T_M70L_L84F_A106V_D108N_H123Y_D147Y_149V_E155V_1156F), (N37S_L84F_A106V_D108N_H123Y_D147Y_E155V_1156F_K16IT), (H36L_L84F_A106V_D108N_H123Y_D147Y_Q154H_E155V_1156F), (N72S_L84F_A106V_D108N_H123Y_S146R_D147Y_E155V_1156F), (H36L_P48L_L84F_A106V_D108N_H123Y_E134G_D147Y_E155V_1156F), (H36L_L84F_A106V_D108N_H123Y_D147Y_E155V_1156F_K157N), (H36L_L84F_A106V_D108N_H123Y_S146C_D147Y_E155V_1156F), (L84F_A106V_D108N_H123Y_S146R_D147Y_E155V_1156F_K16IT), (N37S_R51H_D77G_L84F_A106V_D108N_H123Y_D147Y_E155V_1156F), (R51L_L84F_A106V_D108N_H123Y_D147Y_E155V_1156F_K157N), (D24G_Q71R_L84F_H96L_A106V_D108N_H123Y_D147Y_E155V_1156F_K160E), (H36L_G67V_L84F_A106V_D108N_H123Y_S146T_D147Y_E155V_1156F), (Q71L_L84F_A106V_D108N_H123Y_L137M_A143E_D147Y_E155V_1156F), (E25G_L84F_A106V_D108N_H123Y_D147Y_E155V_1156F_Q159L), (L84F_A91T_F104I_A106V_D108N_H123Y_D147Y_E155V_1156F), (N72D_L84F_A106V_D108N_H123Y_G125A_D147Y_E155V_I156F), (P48S_L84F_S97C_A106V_D108N_H123Y_D147Y_E155V_1156F), (W23G_L84F_A106V_D108N_H123Y_D147Y_E155V_1156F), (D24G_P48L_Q71R_L84F_A106V_D108N_H123Y_D147Y_E155V_1156F_Q159L), (L84F_A106V_D108N_H123Y_A142N_D147Y_E155V_I156F), (H36L_R51L_L84F_A106V_D108N_H123Y_A142N_S146C_D147Y_E155V_1156F _K157N), (N37S_L84F_A106V_D108N_H123Y_A142N_D147Y_E155V_1156F_K161T), (L84F_A106V_D108N_D147Y_E155V_I156F), (R51L_L84F_A106V_D108N_H123Y_S146C_D147Y_E155V_I156F_K157N_K161T), (L84F_A106V_D108N_H123Y_S146C_D147Y_E155V_1156F_K161T), (L84F_A106V_D108N_H123Y_S146C_D147Y_E155V_1156F_K157N_K160E_K161T), (L84F_A106V_D108N_H123Y_S146C_D147Y_E155V_1156F_K157N_K160E), (R74Q L84F_A106V_D108N_H123Y_D147Y_E155V_1156F), (R74A_L84F_A106V_D108N_H123Y_D147Y_E155V_1156F), (L84F_A106V_D108N_H123Y_D147Y_E155V_1156F), (R74Q_L84F_A106V_D108N_H123Y_D147Y_E155V_1156F), (L84F_R98Q_A106V_D108N_H123Y_D147Y_E155V_1156F), (L84F_A106V_D108N_H123Y_R129Q_D147Y_E155V_1156F), (P48S_L84F_A106V_D108N_H123Y_A142N_D147Y_E155V_I156F), (P48S_A142N), (P48T_149V_L84F_A106V_D108N_H123Y_A142N_D147Y_E155V_1156F_L157N), (P48T_I49V_A142N), (H36L_P48S_R51L_L84F_A106V_D108N_H123Y_S146C_D147Y_E155V_1156F K157N), (H36L_P48S_R51L_L84F_A106V_D108N_H123Y_S146C_A142N_D147Y_E155V_1156F K157N), (H36L_P48T_I49V_R51L_L84F_A106V_D108N_H123Y_S146C_D147Y_E155V_1156F K157N), (H36L_P48T_149V_R51L_L84F_A106V_D108N_H123Y_A142N_S146C_D147Y_E155V I156F_K157N), (H36L_P48A_R51L_L84F_A106V_D108N_H123Y_S146C_D147Y_E155V_1156F K157N), (H36L_P48A_R51L_L84F_A106V_D108N_H123Y_A142N_S146C_D147Y_E155V_1156F K157N), (H36L_P48A_R51L_L84F_A106V_D108N_H123Y_S146C_A142N_D147Y_E155V_1156F _K157N), (W23L_H36L_P48A_R51L_L84F_A106V_D108N_H123Y_S146C_D147Y_E155V_1156F _K157N), (W23R_H36L_P48A_R51L_L84F_A106V_D108N_H123Y_S146C_D147Y_E155V_1156F K157N), (W23L_H36L_P48A_R51L_L84F_A106V_D108N_H123Y_S146R_D147Y_E155V_1156F K161T), (H36L_P48A_R51L_L84F_A106V_D108N_H123Y_S146C_D147Y_R152H_E155V_1156F K157N), (H36L_P48A_R51L_L84F_A106V_D108N_H123Y_S146C_D147Y_R152P_E155V_1156F K157N), (W23L_H36L_P48A_R51L_L84F_A106V_D108N_H123Y_S146C_D147Y_R152P_E155V I156F_K157N), (W23L_H36L_P48A_R51L_L84F_A106V_D108N_H123Y_A142A_S146C_D147Y_E155 V_I156F_K157N), (W23L_H36L_P48A_R51L_L84F_A106V_D108N_H123Y_A142A_S146C_D147Y_R152P E155V_I156F_K157N), (W23L_H36L_P48A_R51L_L84F_A106V_D108N_H123Y_S146R_D147Y_E155V_1156F K161T), (W23R_H36L_P48A_R51L_L84F_A106V_D108N_H123Y_S146C_D147Y_R152P_E155V I156F_K157N), (H36L_P48A_R51L_L84F_A106V_D108N_H123Y_A142N_S146C_D147Y_R152P_E155 V_I156F_K157N).

In some embodiments, the adenosine deaminase comprises an amino acid sequence that is at least 60%, 65%, 70%, 75%, 80%, 85%, 90%, 95, 98%, 99%, or 99.5% identical to any one of SEQ ID NOs: 15-23, or any of the adenosine deaminases provided herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth in SEQ ID NO: 15 or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 166, identical contiguous amino acid residues as compared to any one of the amino acid sequences set forth in SEQ ID NO: 15 or any of the adenosine deaminases provided herein. In some embodiments, the adenosine deaminase comprises the amino acid sequence of SEQ ID NO: 15 or any of the adenosine deaminases provided herein. In some embodiments, the adenosine deaminase consists of the amino acid sequence of SEQ ID NO: 15 or any of the adenosine deaminases provided herein.

### Nuclear Localization Signals

In various embodiments, the base editors disclosed herein further comprise one or more, preferably at least two nuclear localization signals. In a preferred embodiment, the base editors comprise at least two NLSs. In embodiments with at least two NLSs, the NLSs can be the same NLSs or they can be different NLSs. In addition, the NLSs may be expressed as part of a fusion protein with the remaining portions of the base editors. The location of the NLS fusion can be at the N-terminus, the C-terminus, or within a sequence of a base editor (e.g., inserted between the encoded napR/DNAbp component (e.g., Cas9) and a DNA effector moiety (e.g., a deaminase)).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. A nuclear localization signal can also target the exterior surface of a cell. Thus, a single nuclear localization signal can direct the entity with which it is associated to the exterior of a cell and to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., international PCT application, PCT/EP2000/011690, filed Nov. 23, 2000, published asWO/2001/038547 on May 31, 2001, the contents of which are incorporated herein by reference for their disclosure of exemplary nuclear localization sequences. In some embodiments, a NLS comprises the amino acid sequence PKKKRKV (SEQ ID NO: 115), MDSLLMNRRKFLYQFKNVRWAKGRRETYLC (SEQ ID NO: 116), KRTADGSEFESPKKKRKV (SEQ ID NO: 1), or KRTADGSEFEPKKKRKV (SEQ ID NO: 2).

In one aspect of the invention, a base editor (e.g., a known base editor, such as BE1, BE2, BE3, or BE4) may be modified with one or more nuclear localization signals (NLS), preferably at least two NLSs. In preferred embodiments, the base editors are modified with two or more NLSs. The invention contemplates the use of any nuclear localization signal known in the art at the time of the invention, or any nuclear localization signal that is identified or otherwise made available in the state of the art after the time of the instant filing. A representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273:14731-37, incorporated herein by reference) to eight amino acids, and is typically rich in lysine and arginine residues (Magin et al., (2000) Virology 274:11-16, incorporated herein by reference). Nuclear localization signals often comprise proline residues. A variety of nuclear localization signals have been identified and have been used to effect transport of biological molecules from the cytoplasm to the nucleus of a cell. Sec, e.g., Tinland et al., (1992) Proc. Natl. Acad. Sci. U.S.A. 89:7442-46; Moede et al., (1999) FEBS Leff. 461:229-34, which is incorporated by reference. Translocation is currently thought to involve nuclear pore proteins.

Most NLSs can be classified in three general groups: (i) a monopartite NLS exemplified by the SV40 large T antigen NLS (PKKKRKV SEQ ID NO: 115); (ii) a bipartite motif consisting of two basic domains separated by a variable number of spacer amino acids and exemplified by the Xenopus nucleoplasmin NLS (KRXXXXXXXXXXKKKL SEQ ID NO: 119); and (iii) noncanonical sequences such as M9 of the hnRNP A1 protein, the influenza virus nucleoprotein NLS, and the yeast Gal4 protein NLS (Dingwall and Laskey 1991).

Nuclear localization signals appear at various points in the amino acid sequences of proteins. NLS's have been identified at the N-terminus, the C-terminus and in the central region of proteins. Thus, the specification provides base editors that may be modified with one or more NLSs at the C-terminus, the N-terminus, as well as at in internal regaion of the base editor. The residues of a longer sequence that do not function as component NLS residues should be selected so as not to interfere, for example tonically or sterically, with the nuclear localization signal itself. Therefore, although there are no strict limits on the composition of an NLS-comprising sequence, in practice, such a sequence can be functionally limited in length and composition.

The present disclosure contemplates any suitable means by which to modify a base editor to include one or more NLSs. In one aspect, the base editors can be engineered to express a base editor protein that is translationally fused at its N-terminus or its C-terminus (or both) to one or more NLSs, i.e., to form a base editor-NLS fusion construct. In other embodiments, the base editor-encoding nucleotide sequence can be genetically modified to incorporate a reading frame that encodes one or more NLSs in an internal region of the encoded base editor. In addition, the NLSs may include various amino acid linkers or spacer regions encoded between the base editor and the N-terminally, C-terminally, or internally-attached NLS amino acid sequence, e.g, and in the central region of proteins. Thus, the present disclosure also provides for nucleotide constructs, vectors, and host cells for expressing fusion proteins that comprise a base editor and one or more NLSs.

The improved base editors described herein may also comprise nuclear localization signals which are linked to a base editor through one or more linkers, e.g., and polymeric, amino acid, nucleic acid, polysaccharide, chemical, or nucleic acid linker element. The linkers within the contemplated scope of the disclosure are not intented to have any limitations and can be any suitable type of molecule (e.g., polymer, amino acid, polysaccharide, nucleic acid, lipid, or any synthetic chemical linker moiety) and be joined to the base editor by any suitable strategy that effectuates forming a bond (e.g., covalent linkage, hydrogen bonding) between the base editor and the one or more NLSs.

### Additional Functionalities

The improved base editors described herein also may include one or more additional functionalities. In certain embodiments, the additional functionalities may include an effector of base repair.

In certain embodiments, the base editors described herein may comprise an inhibitor of base repair. The term “inhibitor of base repair” or “IBR” refers to a protein that is capable in inhibiting the activity of a nucleic acid repair enzyme, for example a base excision repair enzyme. In some embodiments, the IBR is an inhibitor of inosine base excision repair. Exemplary inhibitors of base repair include inhibitors of APE1, Endo III, Endo IV, Endo V, Endo VIII, Fpg, hOGG1, hNEIL1, T7 EndoI, T4PDG, UDG, hSMUG1, and hAAG. In some embodiments, the IBR is an inhibitor of Endo V or hAAG. In some embodiments, the IBR is a catalytically inactive EndoV or a catalytically inactive hAAG.

In other embodiments, the base editors described herein may comprise a uracil glycosylase inhibitor. The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 10. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 10. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 10. In some embodiments, a UGI comprises an amino acid sequence homologous to the amino acid sequence set forth in SEQ ID NO: 10, or an amino acid sequence homologous to a fragment of the amino acid sequence set forth in SEQ ID NO: 10. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 10. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 10. In some embodiments, the UGI comprises the following amino acid sequence:

>sp|P14739|UNGI_BPPB2 Uracil-DNA glycosylase inhibitor MTNLSDIIEKETGKQLVIQESILMLPEEVEEVIGNKPESDILVHTAYDESTDENVMLLT SDAPEYKPWALVIQDSNGENKIKML (SEQ ID NO: 10).

In some embodiments, the base editor described herein may comprise one or more heterologous protein domains (e.g., about or more than about 1, 2, 3, 4, 5, 6, 7, 8, 9, 10, or more domains in addition to the base editor components). A base editor may comprise any additional protein sequence, and optionally a linker sequence between any two domains. Examples of protein domains that may be fused to a base editor or component thereof (e.g., the napR/DNAbp moiety, the nucleic acid effector moiety, or the NLS moeity) include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A base editor may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein comprising a base editor are described in US20110059502, incorporated herein by reference. In some embodiments, a tagged base editor is used to identify the location of a target sequence.

In an aspect of the invention, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into a cell to encode a gene product which serves as a marker by which to measure the alteration or modification of expression of the gene product. In a further embodiment of the invention, the DNA molecule encoding the gene product may be introduced into the cell via a vector. In a preferred embodiment of the invention the gene product is luciferase. In a further embodiment of the invention the expression of the gene product is decreased.

Other exemplary features that may be present are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

### The Guide Sequence (e.g., a Guide RNA)

In various embodiments, the improved base editors can be complexed, bound, or otherwise associated with (e.g., via any type of covalent or non-covalent bond) one or more guide sequences, i.e., the sequence which becomes associated or bound to the base editor and directs its localization to a specific target sequence having complementarity to the guide sequence or a portion thereof. The particular design aspects of a guide sequence will depend upon the nucleotide sequence of a genomic target site of interest (i.e., the desired site to be edited) and the type of napR/DNAbp (e.g., type of Cas protein) present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a napR/DNAbp (e.g., a Cas9, Cas9 homolog, or Cas9 variant) to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site of the form MMMMMMMMNNNNNNNNNNNNXGG (SEQ ID NO: 121) where NNNNNNNNNNNNXGG (SEQ ID NO: 122) (N is A, G, T, or C; and X can be anything) has a single occurrence in the genome. A unique target sequence in a genome may include an S. pyogenes Cas9 target site of the form MMMMMMMMMNNNNNNNNNNNXGG (SEQ ID NO: 123) where NNNNNNNNNNNXGG (SEQ ID NO: 124) (N is A, G, T, or C; and X can be anything) has a single occurrence in the genome. For the S. thermophilus CRISPR1Cas9, a unique target sequence in a genome may include a Cas9 target site of the form MMMMMMMMNNNNNNNNNNNNXXAGAAW (SEQ ID NO: 125) where NNNNNNNNNNNNXXAGAAW (SEQ ID NO: 126) (N is A, G, T, or C; X can be anything; and W is A or T) has a single occurrence in the genome. A unique target sequence in a genome may include an S. thermophilus CRISPR 1 Cas9 target site of the form MMMMMMMMMNNNNNNNNNNNXXAGAAW (SEQ ID NO: 127) where NNNNNNNNNNNXXAGAAW (SEQ ID NO: 128) (N is A, G, T, or C; X can be anything; and W is A or T) has a single occurrence in the genome. For the S. pyogenes Cas9, a unique target sequence in a genome may include a Cas9 target site of the form MMMMMMMMNNNNNNNNNNNNXGGXG (SEQ ID NO: 129) where NNNNNNNNNNNNXGGXG (SEQ ID NO: 130) (N is A, G, T, or C; and X can be anything) has a single occurrence in the genome. A unique target sequence in a genome may include an S. pyogenes Cas9 target site of the form MMMMMMMMMNNNNNNNNNNNXGGXG (SEQ ID NO: 131) where NNNNNNNNNNNXGGXG (SEQ ID NO: 132) (N is A, G, T, or C; and X can be anything) has a single occurrence in the genome. In each of these sequences “M” may be A, G, T, or C, and need not be considered in identifying a sequence as unique.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106 (1): 23-24; and PA Carr and GM Church, 2009, Nature Biotechnology 27 (12): 1151-62). Further algorithms may be found in U.S. application Ser. No. 61/836,080; Broad Reference BI-2013/004A); incorporated herein by reference.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator: (1) NNNNNNNNgtttttgtactctcaagatttaGAAAtaaatcttgcagaagctacaaagataaggctt catgccgaaatcaacaccctgtcattttatggcagggtgttttcgttatttaaTTTTTT (SEQ ID NO: 133); (2) NNNNNNNNNNNNNNNNNNgtttttgtactctcaGAAAtgcagaagctacaaagataaggcttcatgcc gaaatca acaccctgtcattttatggcagggtgttttcgttatttaaTTTTTT (SEQ ID NO: 134); (3) NNNNNNNNNNNNNNNNNNNNgtttttgtactctcaGAAAtgcagaagctacaaagataaggcttcat gccgaaatca acaccctgtcattttatggcagggtgtTTTTT (SEQ ID NO: 135); (4) NNNNNNNNNNNNNNNNNNNNgttttagagctaGAAAtagcaagttaaaataaggctagtccgttatc aacttgaaaa agtggcaccgagtcggtgcTTTTTT (SEQ ID NO: 136); (5) NNNNNNNNNNNNNNNNNNNNgttttagagctaGAAATAGcaagttaaaataaggctagtccgttat caacttgaa aaagtgTTTTTTT (SEQ ID NO: 137); and (6) NNNNNNNNNNNNNNNNNNNNgttttagagctagAAATAGcaagttaaaataaggctagtccgttat caTTTTT TTT (SEQ ID NO: 138). In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

It will be apparent to those of skill in the art that in order to target any of the fusion proteins comprising a Cas9 domain and an adenosine deaminase, as disclosed herein, to a target site, e.g., a site comprising a point mutation to be edited, it is typically necessary to co-express the fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9: nucleic acid editing enzyme/domain fusion protein.

In some embodiments, the guide RNA comprises a structure 5′-[guide sequence]-guuuuagagcuagaaauagcaaguuaaaauaaaggcuaguccguuaucaacuugaaaaaguggcaccgagucggugcuu uuu-3′ (SEQ ID NO: 139), wherein the guide sequence comprises a sequence that is complementary to the target sequence. The guide sequence is typically 20 nucleotides long. The sequences of suitable guide RNAs for targeting Cas9: nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein. Additional guide sequences are are well known in the art and can be used with the base editors described herein.

### Linkers

In certain embodiments, linkers may be used to link any of the peptides or peptide domains or moieties of the invention (e.g., moiety A covalently linked to moiety B which is covalently linked to moiety C).

As defined above, the term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a recombinase. In some embodiments, a linker joins a dCas9 and base editor moiety (e.g., a cytidine or adenosine deaminase). Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated

The linker may be as simple as a covalent bond, or it may be a polymeric linker many atoms in length. In certain embodiments, the linker is a polpeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included funtionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some other embodiments, the linker comprises the amino acid sequence (GGGGS) n (SEQ ID NO: 110), (G) n (SEQ ID NO: 118), (EAAAK) n (SEQ ID NO: 111), (GGS) n (SEQ ID NO: 112), (SGGS) n (SEQ ID NO: 113), SGSETPGTSESATPES SEQ ID NO: 114), (XP) n (SEQ ID NO: 120), or any combination thereof, wherein n is independently an integer between 1 and 30, and wherein X is any amino acid. In some embodiments, the linker comprises the amino acid sequence (GGS) n (SEQ ID NO: 112), wherein n is 1, 3, or 7. In some embodiments, the linker comprises the amino acid sequence SGSETPGTSESATPES (SEQ ID NO: 114).

In some embodiments, the fusion protein comprises the structure [nucleic acid editing domain]-[optional linker sequence]-[dCas9 or Cas9 nickase]-[optional linker sequence]-[UGI]. In some embodiments, the fusion protein comprises the structure [nucleic acid editing domain]-[optional linker sequence]-[UGI]-[optional linker sequence]-[dCas9 or Cas9 nickase]; [UGI]-[optional linker sequence]-[nucleic acid editing domain]-[optional linker sequence]-[dCas9 or Cas9 nickase]; [UGI]-[optional linker sequence]-[dCas9 or Cas9 nickase]-[optional linker sequence]-[nucleic acid editing domain]; [dCas9 or Cas9 nickase]-[optional linker sequence]-[UGI]-[optional linker sequence]-[nucleic acid editing domain]; or [dCas9 or Cas9 nickase]-[optional linker sequence]-[nucleic acid editing domain]-[optional linker sequence]-[UGI].

### Improved Editing Efficiencies

As exemplified in the Examples, the efficiency of base editing may be increased by the various approaches described herein for improving base editors in cells. In one aspect, base editing efficiency may be increased by optimizing base editor codon usage which increases base editor mRNA expression levels thereby increase base editing efficiencies. In another aspect, base editing efficiency may be increased by optimizing base editor amino acid sequences through ancestral sequence reconstruction. In still other aspects, base editing efficiency may be increased by modifying base editors to include at least two NLSs, e.g., wherein one is located at the N-terminus and another (same or difference NLS) is located as the C-terminus of a base editor fusion protein. The level or degree of increase in efficiency may be measured or expressed in any suitable manner such as the percentage of nucleotides correctly edited from the total number of nucleotides attempted to be edited by a base editor described herein.

The base editors in various embodiment may be characterized with an improved editing capability that is at least 0.5-fold, or at least 0.6-fold, or at least 0.7-fold, or at least 0.8-fold, or at least 0.9-fold, or at least 1.0-fold, or at least 1.5-fold, or at least 2.0-fold, or at least 3.0-fold, or at least 4.0-fold, or at least 5.0-fold, or at least 6.0-fold, or at least 7.0-fold, or at least 8.0-fold, or at least 9.0-fold, or at least 10.0-fold, or at least 20-, 30-, 40-, 50-, 60-, 70-, 80-, 90-, or 100-fold or more higher than the base editing efficiency of a base editor that has not been modified by at least one of the modification approached described herein (e.g., codon optimization, at least 2 NLSs, or by ancestral sequence reconstruction. Examples of specific increases in base editing efficiencies are exemplified in the Examples.

### II. Methods for Making the Improved Base-Editors

Despite recent advances in the design of base editors, the efficiency of base editing varies widely. To increase base editing efficiency, the inventors sought to identify the factors that limit base editing efficiency in cells. It was surprisingly found by the inventors that expression and nuclear localization in human cells imposed key bottlenecks on editing efficiency. The inventors discovered that by optimizing codon usage, using improved nuclear localization sequences (NLSs), and performing ancestral reconstruction of deaminases resulted in base editors with greatly increased editing efficiency, often more than doubling target nucleotide conversion yields as compared to the unmodified counterpart editors. The resulting base editors were shown, as demonstrated in the Examples, to install point mutations relevant to human disease in a variety of mammalian cell types much more efficiently than previously described base editors. These methods can be used to provide improved base editors that can be used to efficiently edit a nucleic acid molecule in a manner that is dramatically improved as compared to base editors known in the art. The improved base editors may be used to efficiently edit nucleic acid molecules, e.g., a genome, for example, by correcting a disease-causing point mutation.

Thus, the invention relates in various aspects to methods of making the disclosed improved base editors by various modes of manipulation that include but are not limited to codon optimization and performance of ancestral reconstruction of components of the base editors (e.g., of a deaminase) to achieve greater expression levels in a cell, and the use of nuclear localization sequences (NLS) s, preferably at least two NLSs to increase the localization of the expressed base editors into a cell nucleus.

### Increasing Expression

The base editors contemplated herein can include modifications that result in increased expression through codon optimization and ancestral reconstruction analysis.

In some embodiments, the base editors (or a component thereof) is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database”, and these tables can be adapted in a number of ways. Sec Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme correspond to the most frequently used codon for a particular amino acid.

In other embodiments, the base editors of the invention have improved expression (as compared to non-modified or state of the art counterpart editors) as a result of ancestral sequence reconstruction analysis. Ancestral sequence reconstruction (ASR) is the process of analyzing modern sequences within an evolutionary/phylogenetic context to infer the ancestral sequences at particular nodes of a tree. These ancient sequences are most often then synthesized, recombinantly expressed in laboratory microorganisms or cell lines, and then characterized to reveal the ancient properties of the extinct biomolecules 2,3,4,5,6. This process has produced tremendous insights into the mechanisms of molecular adaptation and functional divergence7. Despite such insights, a major criticism of ASR is the general inability to benchmark accuracy of the implemented algorithms. It is difficult to benchmark ASR for many reasons. Notably, genetic material is not preserved in fossils on a long enough time scale to satisfy most ASR studies (many millions to billions of years ago), and it is not yet physically possible to travel back in time to collect samples. Reference can be made to Cai et al., “Reconstruction of ancestral protein sequences and its applications,” BMC Evolutionary Biology 2004, 4:33 and Zakas et al., “Enhancing the pharmaceutical properties of protein drugs by ancestral sequence reconstruction,” Nature Biotechnology, 35, pp. 35-37 (2017), each of which are incorporated herein by reference.

There are many software packages available which can perform ancestral state reconstruction. Generally, these software packages have been developed and maintained through the efforts of scientists in related fields and released under free software licenses. The following list is not meant to be a comprehensive itemization of all available packages, but provides a representative sample of the extensive variety of packages that implement methods of ancestral reconstruction with different strengths and features: PAML (Phylogenetic Analysis by Maximum Likelihood, available at//abacus.gene.ucl.ac.uk/software/paml.html), BEAST (Bayesian evolutionary analysis by sampling trees, available at//www.beast2.org/wiki/index.php/Main_Page), and Diversitree (FitzJohn RG, 2012. Diversitree: comparative phylogenetic analyses of diversification in R. Methods in Ecology and Evolution), and HyPHy (Hypothesis testing using phylogenies, available at//hyphy.org/w/index.php/Main_Page).

The Examples demonstrate one embodiment for using ASR to increase overall expression of base editors disclosed herein.

The above description is meant to be non-limiting with regard to making base editors having increased expression, and thereby increase editing efficiencies.

### Increasing Nuclear Localization

In one aspect, the specification provides a strategy for improving a base editor by incorporating one or more nuclear localization signals (NLS) therein, e.g., as a N-terminal or C-terminal fusion protein. Preferably, at least two NLSs are incorporated into a base editor. In the Examples, the inventors explored whether sub-optimal nuclear localization could be a basis or poor editing efficiency. The inventors test six combinations of the base editor “BE4” as N- and/or C-terminal fusions to either the SV40 NLS or the bipartite NLS (bpNLS). As shown in the Examples, all the variants using one or two bpNLSs showed improvements in editing efficiency. The presence of a bpNLS at both the N- and C-terminus (referred to hereafter as “bis-bpNLS”) performed best, resulting in a 1.3-fold average improvement in BE4-mediated C·G-to-T·A editing efficiency at five exemplary tested genomic loci (48+8.0% average editing compared to 37+5.6% for the C-terminal SV40 NLS used in BE4). These results together suggest that modifying base editors with one or more nuclear localization signals, e.g., a bis-bpNLS, can significantly improve the editing efficiency of previously described for known base editors, such as, BE3 and BE4 (6, 7).

However, the Examples are not intended to be limiting, but only demonstrative of wider strategy for improving base editor efficiency through the modification of the base editor with one or more nuclear localization signals, preferably at least two NLSs. The invention is not intended to be limiting with regard to which NLS is employed, and the manner by which the NLS is attached to or otherwise coupled to a base editor. NLS sequences are known in the art and examples are disclosed herein.

### Vectors

Several aspects of the making and using the base editors of the invention relate to vector systems comprising one or more vectors, or vectors as such. Vectors can be designed to clone and/or express the improved base editors of the disclosure. Vectors can also be designed to transfect the improved base editors of the disclosure into one or more cells, e.g., a target diseased eukaryotic cell for treatment with the base editor systems and methods disclosed herein.

Vectors can be designed for expression of base editor transcripts (e.g. nucleic acid transcripts, proteins, or enzymes) in prokaryotic or eukaryotic cells. For example, base editor transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press. San Diego, Calif. (1990). Alternatively, expression vectors encoding one or more improved base editors described herein can be transcribed and translated in vitro, for example using T7 promoter regulatory sequences and T7 polymerase.

Vectors may be introduced and propagated in a prokaryotic cells. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins.

Fusion expression vectors also may be used to express the improved base editors of the disclosure. Such vectors generally add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector for expressing the improved base editors described herein. Examples of vectors for expression in yeast Saccharomyces cerivisae include pYepSecl (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (In Vitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39).

In some embodiments, a vector is capable of driving expression of one or more sequences in mammalian cells using a mammalian expression vector. Examples of mammalian expression vectors include pCDM8 (Seed, 1987. Nature 329:840) and pMT2PC (Kaufman, et al., 1987. EMBO J. 6:187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd cd., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Banciji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter, U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546).

### Increasing Base Editor Efficiencies

Some aspects of the disclosure are based on the recognition that any of the base editors provided herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate or deaminate) a specific nucleotide within a nucleic acid, without generating a large number of insertions or deletions (i.e., indels) in the nucleic acid. In certain embodiments, any of the base editors provided herein are capable of generating a greater proportion of intended modifications (e.g., point mutations or deaminations) versus indels. In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method, for example the methods used in the below Examples. In some embodiments, to calculate indel frequencies, sequencing reads are scanned for exact matches to two 10-bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the base editors provided herein are capable of limiting formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation, such as a point mutation, in a nucleic acid (e.g. a nucleic acid within a genome of a subject) without generating a significant number of unintended mutations, such as unintended point mutations. In some embodiments, a intended mutation is a mutation that is generated by a specific base editor bound to a gRNA, specifically designed to generate the intended mutation. In some embodiments, the intended mutation is a mutation associated with a disease or disorder. In some embodiments, the intended mutation is a adenine (A) to guanine (G) point mutation associated with a disease or disorder. In some embodiments, the intended mutation is a thymine (T) to cytosine (C) point mutation associated with a disease or disorder. In some embodiments, the intended mutation is a adenine (A) to guanine (G) point mutation within the coding region of a gene. In some embodiments, the intended mutation is a thymine (T) to cytosine (C) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended point mutations: unintended point mutations) that is greater than 1:1. In some embodiments, any of the base editors provided herein are capable of generating a ratio of intended mutations to unintended mutations (e.g., intended point mutations: unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described in the “Base Editor Efficiency” section, herein, may be applied to any of the fusion proteins, or methods of using the fusion proteins provided herein.

### III. Methods of Using Improved Base-Editors

Some aspects of this disclosure provide methods of using the improved base editors disclosed herein, or base editor complexes comprising one or more napR/DNAbp-programming nucleic acid molecules (e.g., Cas9 guide RNAs) and a nucleobase editor provided herein.

### Editing DNA or RNA

Some aspects of the disclosure provide methods for editing a nucleic acid using the base editors described herein. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to an adenosine deaminase) and a guide nucleic acid (e.g., gRNA), wherein the target region comprises a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase. In some embodiments, the method results in less than 20% indel formation in the nucleic acid. It should be appreciated that in some embodiments, step b is omitted. In some embodiments, the first nucleobase is an adenine. In some embodiments, the second nucleobase is a deaminated adenine, or inosine. In some embodiments, the third nucleobase is a thymine. In some embodiments, the fourth nucleobase is a cytosine. In some embodiments, the method results in less than 19%, 18%, 16%, 14%, 12%, 10%, 8%, 6%, 4%, 2%, 1%, 0.5%, 0.2%, or less than 0.1% indel formation. In some embodiments, the method further comprises replacing the second nucleobase with a fifth nucleobase that is complementary to the fourth nucleobase, thereby generating an intended edited base pair (e.g., A: T to G: C). In some embodiments, the fifth nucleobase is a guanine. In some embodiments, at least 5% of the intended base pairs are edited. In some embodiments, at least 10%, 15%, 20%, 25%, 30%, 35%, 40%, 45%, or 50% of the intended base pairs are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine. In some embodiments, the first base is adenine. In some embodiments, the second base is not a G, C, A, or T. In some embodiments, the second base is inosine. In some embodiments, the base editor inhibits base excision repair of the edited strand. In some embodiments, the base editor protects or binds the non-edited strand. In some embodiments, the base editor comprises UGI activity. In some embodiments, the base editor comprises a catalytically inactive inosine-specific nuclease. In some embodiments, the base editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors provided herein. In some embodiments, a target window is a deamination window.

In some embodiments, the disclosure provides methods for editing a nucleotide. In some embodiments, the disclosure provides a method for editing a nucleobase pair of a double-stranded DNA sequence. In some embodiments, the method comprises a) contacting a target region of the double-stranded DNA sequence with a complex comprising a base editor and a guide nucleic acid (e.g., gRNA), where the target region comprises a target nucleobase pair, b) inducing strand separation of said target region, c) converting a first nucleobase of said target nucleobase pair in a single strand of the target region to a second nucleobase, d) cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, thereby generating an intended edited base pair, wherein the efficiency of generating the intended edited base pair is at least 5%. It should be appreciated that in some embodiments, step b is omitted. In some embodiments, at least 5% of the intended base pairs are edited. In some embodiments, at least 10%, 15%, 20%, 25%, 30%, 35%, 40%, 45%, or 50% of the intended base pairs are edited. In some embodiments, the method causes less than 19%, 18%, 16%, 14%, 12%, 10%, 8%, 6%, 4%, 2%, 1%, 0.5%, 0.2%, or less than 0.1% indel formation. In some embodiments, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand is hybridized to the guide nucleic acid. In some embodiments, the cut single strand is opposite to the strand comprising the first nucleobase. In some embodiments, the first base is adenine. In some embodiments, the second nucleobase is not G, C, A, or T. In some embodiments, the second base is inosine. In some embodiments, the base editor inhibits base excision repair of the edited strand. In some embodiments, the base editor protects (e.g., form base excision repair) or binds the non-edited strand. In some embodiments, the nucleobase editor comprises UGI activity. In some embodiments, the base editor comprises a catalytically inactive inosine-specific nuclease. In some embodiments, the nucleobase editor comprises nickase activity. In some embodiments, the intended edited base pair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site. In some embodiments, the nucleobase editor comprises a linker. In some embodiments, the linker is 1-25 amino acids in length. In some embodiments, the linker is 5-20 amino acids in length. In some embodiments, the linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair occurs within the target window. In some embodiments, the target window comprises the intended edited base pair. In some embodiments, the nucleobase editor is any one of the base editors provided herein.

In another embodiment, the disclosure provides editing methods comprising contacting a DNA, or RNA molecule with any of the base editors provided herein, and with at least one guide nucleic acid (e.g., guide RNA), wherein the guide nucleic acid, (e.g., guide RNA) is about 15-100 nucleotides long and comprises a sequence of at least 10 contiguous nucleotides that is complementary to a target sequence. In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to an AGC, GAG, TTT, GTG, or CAA sequence.

In some embodiments, the target DNA sequence comprises a sequence associated with a disease or disorder. In some embodiments, the target DNA sequence comprises a point mutation associated with a disease or disorder. In some embodiments, the activity of the fusion protein (e.g., comprising an adenosine deaminase and a Cas9 domain), or the complex, results in a correction of the point mutation. In some embodiments, the target DNA sequence comprises a G->A point mutation associated with a disease or disorder, and wherein the deamination of the mutant A base results in a sequence that is not associated with a disease or disorder. In some embodiments, the target DNA sequence encodes a protein, and the point mutation is in a codon and results in a change in the amino acid encoded by the mutant codon as compared to the wild-type codon. In some embodiments, the deamination of the mutant A results in a change of the amino acid encoded by the mutant codon. In some embodiments, the deamination of the mutant A results in the codon encoding the wild-type amino acid. In some embodiments, the contacting is in vivo in a subject. In some embodiments, the subject has or has been diagnosed with a disease or disorder. In some embodiments, the disease or disorder is phenylketonuria, von Willebrand disease (vWD), a neoplastic disease associated with a mutant PTEN or BRCA1, or Li-Fraumeni syndrome. A list of exemplary diseases and disorders that may be treated using the base editors described herein is shown in Table 1. Table 1 includes the target gene, the mutation to be corrected, the related disease and the nucleotide sequence of the associated protospacer and PAM.

TABLE 1
List of exemplary diseases that may be treated using the base editors
described herein. The A to be edited in the protospacer is indicated
by underlining and the PAM is indicated in bold.

| Target | | ATCC Cell | | |
|---|---|---|---|---|
| Gene | Mutation | Line | Disease | Protospacer and PAM |
| PTEN | Cys136Tyr | HTB-128 | Cancer | TATATGCATATTTATTACATCGG |
| | | | Predisposition | SEQ ID NO: 140 |
| PTEN | Arg233Ter | HTB-13 | Cancer | CCGTCATGTGGGTCCTGAATTGG |
| | | | Predisposition | SEQ ID NO: 141 |
| TP53 | Glu258Lys | RTB-65 | Cancer | ACACTGAAAGACTCCAGGTCAGG |
| | | | Predisposition | SEQ ID NO: 142 |
| BRCA1 | Gly1738Arg | NA | Cancer | GTCAGAAGAGATGTGGTCAATGG |
| | | | Predisposition | SEQ ID NO: 143 |
| BRCA1 | 4097-1G>A | NA | Cancer | TTTAAAGTGAAGCAGCATCTGGG SEQ ID NO: 144 |
| | | | Predisposition | ATTTAAAGTGAAGCAGCATCTGG SEQ ID NO: 145 |
| PAH | Thr380Met | NA | Phenylketonuria | ACTCCATGACAGTGTAATTTTGG SEQ ID NO: 146 |
| VWF | Ser1285Phe | NA | von Willebrand | GCCTGGAGAAGCCATCCAGCAGG SEQ ID NO: 147 |
| | | | (Hemophilia) | |
| VWF | Arg2535Ter | NA | von Willebrand | CTCAGACACACTCATTGATGAGG SEQ ID NO: 148 |
| | | | (Hemophilia) | |
| TP53 | Arg175His | HCC1395 | Li-Fraumeni | GAGGCACTGCCCCCACCATGAGCG SEQ ID NO: 149 |
| | | | syndrome | |

Some embodiments provide methods for using the improved base editors provided herein. In some embodiments, the base editors are used to introduce a point mutation into a nucleic acid by deaminating a target nucleobase, e.g., an A residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a DNA editing fusion protein to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provided herein is to restore the function of a dysfunctional gene via genome editing. The nucleobase editing proteins provided herein can be validated for gene editing-based human therapeutics in vitro, e.g., by correcting a disease-associated mutation in human cell culture. It will be understood by the skilled artisan that the nucleobase editing proteins provided herein, e.g., the fusion proteins comprising a nucleic acid programmable DNA binding protein (e.g., Cas9) and an adenosine deaminase domain can be used to correct any single point G to A or C to T mutation. In the first case, deamination of the mutant A to I corrects the mutation, and in the latter case, deamination of the A that is base-paired with the mutant T, followed by a round of replication, corrects the mutation. Exemplary point mutations that can be corrected are listed in Tables 1.

The successful correction of point mutations in disease-associated genes and alleles opens up new strategies for gene correction with applications in therapeutics and basic research. Site-specific single-base modification systems like the disclosed fusions of a nucleic acid programmable DNA binding protein and an adenosine deaminase domain also have applications in “reverse” gene therapy, where certain gene functions are purposely suppressed or abolished. In these cases, site-specifically mutating residues that lead to inactivating mutations in a protein, or mutations that inhibit function of the protein can be used to abolish or inhibit protein function

### Methods of Treatment

The instant disclosure provides methods for the treatment of a subject diagnosed with a disease associated with or caused by a point mutation that can be corrected by a DNA editing fusion protein provided herein. For example, in some embodiments, a method is provided that comprises administering to a subject having such a disease, e.g., a cancer associated with a point mutation as described above, an effective amount of an adenosine deaminase fusion protein that corrects the point mutation or introduces a deactivating mutation into a disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

The instant disclosure provides methods for the treatment of additional diseases or disorders, e.g., diseases or disorders that are associated or caused by a point mutation that can be corrected by deaminase-mediated gene editing. Some such diseases are described herein, and additional suitable diseases that can be treated with the strategies and fusion proteins provided herein will be apparent to those of skill in the art based on the instant disclosure. Exemplary suitable diseases and disorders are listed below. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation: 2-methyl-3-hydroxybutyric aciduria; 3 beta-Hydroxysteroid dehydrogenase deficiency; 3-Methylglutaconic aciduria; 3-Oxo-5 alpha-steroid delta 4-dehydrogenase deficiency; 46,XY sex reversal, type 1, 3, and 5; 5-Oxoprolinase deficiency; 6-pyruvoyl-tetrahydropterin synthase deficiency; Aarskog syndrome; Aase syndrome; Achondrogenesis type 2; Achromatopsia 2 and 7; Acquired long QT syndrome; Acrocallosal syndrome, Schinzel type; Acrocapitofemoral dysplasia; Acrodysostosis 2, with or without hormone resistance; Acroerythrokeratoderma; Acromicric dysplasia; Acth-independent macronodular adrenal hyperplasia 2; Activated PI3K-delta syndrome; Acute intermittent porphyria; deficiency of Acyl-CoA dehydrogenase family, member 9; Adams-Oliver syndrome 5 and 6; Adenine phosphoribosyltransferase deficiency; Adenylate kinase deficiency; hemolytic anemia due to Adenylosuccinate lyase deficiency; Adolescent nephronophthisis; Renal-hepatic-pancreatic dysplasia; Meckel syndrome type 7; Adrenoleukodystrophy; Adult junctional epidermolysis bullosa; Epidermolysis bullosa, junctional, localisata variant; Adult neuronal ceroid lipofuscinosis; Adult neuronal ceroid lipofuscinosis; Adult onset ataxia with oculomotor apraxia; ADULT syndrome; Afibrinogenemia and congenital Afibrinogenemia; autosomal recessive Agammaglobulinemia 2; Age-related macular degeneration 3, 6, 11, and 12; Aicardi Goutieres syndromes 1, 4, and 5; Chilbain lupus 1; Alagille syndromes 1 and 2; Alexander disease; Alkaptonuria; Allan-Herndon-Dudley syndrome; Alopecia universalis congenital; Alpers encephalopathy; Alpha-1-antitrypsin deficiency; autosomal dominant, autosomal recessive, and X-linked recessive Alport syndromes; Alzheimer disease, familial, 3, with spastic paraparesis and apraxia; Alzheimer disease, types, 1, 3, and 4; hypocalcification type and hypomaturation type, IIAI Amelogenesis imperfecta; Aminoacylase 1 deficiency; Amish infantile epilepsy syndrome; Amyloidogenic transthyretin amyloidosis; Amyloid Cardiomyopathy, Transthyretin-related; Cardiomyopathy; Amyotrophic lateral sclerosis types 1, 6, 15 (with or without frontotemporal dementia), 22 (with or without frontotemporal dementia), and 10; Frontotemporal dementia with TDP43 inclusions, TARDBP-related; Andermann syndrome; Andersen Tawil syndrome; Congenital long QT syndrome; Anemia, nonspherocytic hemolytic, due to G6PD deficiency; Angelman syndrome; Severe neonatal-onset encephalopathy with microcephaly; susceptibility to Autism, X-linked 3; Angiopathy, hereditary, with nephropathy, aneurysms, and muscle cramps; Angiotensin i-converting enzyme, benign serum increase; Aniridia, cerebellar ataxia, and mental retardation; Anonychia; Antithrombin III deficiency; Antley-Bixler syndrome with genital anomalies and disordered steroidogenesis; Aortic aneurysm, familial thoracic 4, 6, and 9; Thoracic aortic aneurysms and aortic dissections; Multisystemic smooth muscle dysfunction syndrome; Moyamoya disease 5; Aplastic anemia; Apparent mineralocorticoid excess; Arginase deficiency; Argininosuccinate lyase deficiency; Aromatase deficiency; Arrhythmogenic right ventricular cardiomyopathy types 5, 8, and 10; Primary familial hypertrophic cardiomyopathy; Arthrogryposis multiplex congenita, distal, X-linked; Arthrogryposis renal dysfunction cholestasis syndrome; Arthrogryposis, renal dysfunction, and cholestasis 2; Asparagine synthetase deficiency; Abnormality of neuronal migration; Ataxia with vitamin E deficiency; Ataxia, sensory, autosomal dominant; Ataxia-telangiectasia syndrome; Hereditary cancer-predisposing syndrome; Atransferrinemia; Atrial fibrillation, familial, 11, 12, 13, and 16; Atrial septal defects 2, 4, and 7 (with or without atrioventricular conduction defects); Atrial standstill 2; Atrioventricular septal defect 4; Atrophia bulborum hereditaria; ATR-X syndrome; Auriculocondylar syndrome 2; Autoimmune disease, multisystem, infantile-onset; Autoimmune lymphoproliferative syndrome, type 1a; Autosomal dominant hypohidrotic ectodermal dysplasia; Autosomal dominant progressive external ophthalmoplegia with mitochondrial DNA deletions 1 and 3; Autosomal dominant torsion dystonia 4; Autosomal recessive centronuclear myopathy; Autosomal recessive congenital ichthyosis 1, 2, 3, 4A, and 4B; Autosomal recessive cutis laxa type IA and 1B; Autosomal recessive hypohidrotic ectodermal dysplasia syndrome; Ectodermal dysplasia 11b; hypohidrotic/hair/tooth type, autosomal recessive; Autosomal recessive hypophosphatemic bone disease; Axenfeld-Rieger syndrome type 3; Bainbridge-Ropers syndrome; Bannayan-Riley-Ruvalcaba syndrome; PTEN hamartoma tumor syndrome; Baraitser-Winter syndromes 1 and 2; Barakat syndrome; Bardet-Biedl syndromes 1, 11, 16, and 19; Bare lymphocyte syndrome type 2, complementation group E; Bartter syndrome antenatal type 2; Bartter syndrome types 3, 3 with hypocalciuria, and 4; Basal ganglia calcification, idiopathic, 4; Beaded hair; Benign familial hematuria; Benign familial neonatal seizures 1 and 2; Seizures, benign familial neonatal, 1, and/or myokymia; Seizures, Early infantile epileptic encephalopathy 7; Benign familial neonatal-infantile seizures; Benign hereditary chorea; Benign scapuloperoncal muscular dystrophy with cardiomyopathy; Bernard-Soulier syndrome, types A1 and A2 (autosomal dominant); Bestrophinopathy, autosomal recessive; beta Thalassemia; Bethlem myopathy and Bethlem myopathy 2; Bietti crystalline corneoretinal dystrophy; Bile acid synthesis defect, congenital, 2; Biotinidase deficiency; Birk Barel mental retardation dysmorphism syndrome; Blepharophimosis, ptosis, and epicanthus inversus; Bloom syndrome; Borjeson-Forssman-Lehmann syndrome; Boucher Neuhauser syndrome; Brachydactyly types A1 and A2; Brachydactyly with hypertension; Brain small vessel disease with hemorrhage; Branched-chain ketoacid dehydrogenase kinase deficiency; Branchiootic syndromes 2 and 3; Breast cancer, early-onset; Breast-ovarian cancer, familial 1, 2, and 4; Brittle cornea syndrome 2; Brody myopathy; Bronchiectasis with or without elevated sweat chloride 3; Brown-Vialetto-Van lacre syndrome and Brown-Vialetto-Van Lacre syndrome 2; Brugada syndrome; Brugada syndrome 1; Ventricular fibrillation; Paroxysmal familial ventricular fibrillation; Brugada syndrome and Brugada syndrome 4; Long QT syndrome; Sudden cardiac death; Bull eye macular dystrophy; Stargardt disease 4; Cone-rod dystrophy 12; Bullous ichthyosiform erythroderma; Burn-Mckeown syndrome; Candidiasis, familial, 2, 5, 6, and 8; Carbohydrate-deficient glycoprotein syndrome type I and II; Carbonic anhydrase VA deficiency, hyperammonemia due to; Carcinoma of colon; Cardiac arrhythmia; Long QT syndrome, LQTI subtype; Cardioencephalomyopathy, fatal infantile, due to cytochrome c oxidase deficiency; Cardiofaciocutaneous syndrome; Cardiomyopathy; Danon disease; Hypertrophic cardiomyopathy; Left ventricular noncompaction cardiomyopathy; Carnevale syndrome; Carney complex, type 1; Carnitine acylcarnitine translocase deficiency; Carnitine palmitoyltransferase I, II, II (late onset), and II (infantile) deficiency; Cataract 1, 4, autosomal dominant, autosomal dominant, multiple types, with microcornea, coppock-like, juvenile, with microcornea and glucosuria, and nuclear diffuse nonprogressive; Catecholaminergic polymorphic ventricular tachycardia; Caudal regression syndrome; Cd8 deficiency, familial; Central core disease; Centromeric instability of chromosomes 1,9 and 16 and immunodeficiency; Cerebellar ataxia infantile with progressive external ophthalmoplegi and Cerebellar ataxia, mental retardation, and dysequilibrium syndrome 2; Cerebral amyloid angiopathy, APP-related; Cerebral autosomal dominant and recessive arteriopathy with subcortical infarcts and leukoencephalopathy; Cerebral cavernous malformations 2; Cerebrooculofacioskeletal syndrome 2; Cerebro-oculo-facio-skeletal syndrome; Cerebroretinal microangiopathy with calcifications and cysts; Ceroid lipofuscinosis neuronal 2, 6, 7, and 10; Ch\xc3\xa9diak-Higashi syndrome, Chediak-Higashi syndrome, adult type; Charcot-Marie-Tooth disease types 1B, 2B2, 2C, 2F, 2I, 2U (axonal), 1C (demyelinating), dominant intermediate C, recessive intermediate A, 2A2, 4C, 4D, 4H, IF, IVF, and X; Scapuloperoncal spinal muscular atrophy; Distal spinal muscular atrophy, congenital nonprogressive; Spinal muscular atrophy, distal, autosomal recessive, 5; CHARGE association; Childhood hypophosphatasia; Adult hypophosphatasia; Cholecystitis; Progressive familial intrahepatic cholestasis 3; Cholestasis, intrahepatic, of pregnancy 3; Cholestanol storage disease; Cholesterol monooxygenase (side-chain cleaving) deficiency; Chondrodysplasia Blomstrand type; Chondrodysplasia punctata 1, X-linked recessive and 2 X-linked dominant; CHOPS syndrome; Chronic granulomatous disease, autosomal recessive cytochrome b-positive, types 1 and 2; Chudley-Mccullough syndrome; Ciliary dyskinesia, primary, 7, 11, 15, 20 and 22; Citrullinemia type I; Citrullinemia type I and II; Cleidocranial dysostosis; C-like syndrome; Cockayne syndrome type A; Coenzyme Q10 deficiency, primary 1, 4, and 7; Coffin Siris/Intellectual Disability; Coffin-Lowry syndrome; Cohen syndrome; Cold-induced sweating syndrome 1; COLE-CARPENTER SYNDROME 2; Combined cellular and humoral immune defects with granulomas; Combined d-2- and 1-2-hydroxyglutaric aciduria; Combined malonic and methylmalonic aciduria; Combined oxidative phosphorylation deficiencies 1, 3, 4, 12, 15, and 25; Combined partial and complete 17-alpha-hydroxylase/17,20-lyase deficiency; Common variable immunodeficiency 9; Complement component 4, partial deficiency of, due to dysfunctional c1 inhibitor; Complement factor B deficiency; Cone monochromatism; Cone-rod dystrophy 2 and 6; Cone-rod dystrophy amelogenesis imperfecta; Congenital adrenal hyperplasia and Congenital adrenal hypoplasia, X-linked; Congenital amegakaryocytic thrombocytopenia; Congenital aniridia; Congenital central hypoventilation; Hirschsprung disease 3; Congenital contractural arachnodactyly; Congenital contractures of the limbs and face, hypotonia, and developmental delay; Congenital disorder of glycosylation types 1B, ID, 1G, 1H, 1J, 1K, 1N, 1P, 2C, 2J, 2K, Ilm; Congenital dyserythropoietic anemia, type I and II; Congenital ectodermal dysplasia of face; Congenital erythropoietic porphyria; Congenital generalized lipodystrophy type 2; Congenital heart disease, multiple types, 2; Congenital heart disease; Interrupted aortic arch; Congenital lipomatous overgrowth, vascular malformations, and epidermal nevi; Non-small cell lung cancer; Neoplasm of ovary; Cardiac conduction defect, nonspecific; Congenital microvillous atrophy; Congenital muscular dystrophy; Congenital muscular dystrophy due to partial LAMA2 deficiency; Congenital muscular dystrophy-dystroglycanopathy with brain and eye anomalies, types A2, A7, A8, All, and A14; Congenital muscular dystrophy-dystroglycanopathy with mental retardation, types B2, B3, B5, and B15; Congenital muscular dystrophy-dystroglycanopathy without mental retardation, type B5; Congenital muscular hypertrophy-cerebral syndrome; Congenital myasthenic syndrome, acetazolamide-responsive; Congenital myopathy with fiber type disproportion; Congenital ocular coloboma; Congenital stationary night blindness, type 1A, 1B, 1C, 1E, 1F, and 2A; Coproporphyria; Cornea plana 2; Corneal dystrophy, Fuchs endothelial, 4; Corneal endothelial dystrophy type 2; Corneal fragility keratoglobus, blue sclerae and joint hypermobility; Cornelia de Lange syndromes 1 and 5; Coronary artery disease, autosomal dominant 2; Coronary heart disease; Hyperalphalipoproteinemia 2; Cortical dysplasia, complex, with other brain malformations 5 and 6; Cortical malformations, occipital; Corticosteroid-binding globulin deficiency; Corticosterone methyloxidase type 2 deficiency; Costello syndrome; Cowden syndrome 1; Coxa plana; Craniodiaphyseal dysplasia, autosomal dominant; Craniosynostosis 1 and 4; Craniosynostosis and dental anomalies; Creatine deficiency, X-linked; Crouzon syndrome; Cryptophthalmos syndrome; Cryptorchidism, unilateral or bilateral; Cushing symphalangism; Cutaneous malignant melanoma 1; Cutis laxa with osteodystrophy and with severe pulmonary, gastrointestinal, and urinary abnormalities; Cyanosis, transient neonatal and atypical nephropathic; Cystic fibrosis; Cystinuria; Cytochrome c oxidase i deficiency; Cytochrome-c oxidase deficiency; D-2-hydroxyglutaric aciduria 2; Darier disease, segmental; Deafness with labyrinthine aplasia microtia and microdontia (LAMM); Deafness, autosomal dominant 3a, 4, 12, 13, 15, autosomal dominant nonsyndromic sensorineural 17, 20, and 65; Deafness, autosomal recessive 1A, 2, 3, 6, 8, 9, 12, 15, 16, 18b, 22, 28, 31, 44, 49, 63, 77, 86, and 89; Deafness, cochlear, with myopia and intellectual impairment, without vestibular involvement, autosomal dominant, X-linked 2; Deficiency of 2-methylbutyryl-CoA dehydrogenase; Deficiency of 3-hydroxyacyl-CoA dehydrogenase; Deficiency of alpha-mannosidase; Deficiency of aromatic-L-amino-acid decarboxylase; Deficiency of bisphosphoglycerate mutase; Deficiency of butyryl-CoA dehydrogenase; Deficiency of ferroxidase; Deficiency of galactokinase; Deficiency of guanidinoacetate methyltransferase; Deficiency of hyaluronoglucosaminidase; Deficiency of ribose-5-phosphate isomerase; Deficiency of steroid 11-beta-monooxygenase; Deficiency of UDPglucose-hexose-1-phosphate uridylyltransferase; Deficiency of xanthine oxidase; Dejerine-Sottas disease; Charcot-Marie-Tooth disease, types ID and IVF; Dejerine-Sottas syndrome, autosomal dominant; Dendritic cell, monocyte, B lymphocyte, and natural killer lymphocyte deficiency; Desbuquois dysplasia 2; Desbuquois syndrome; DFNA 2 Nonsyndromic Hearing Loss; Diabetes mellitus and insipidus with optic atrophy and deafness; Diabetes mellitus, type 2, and insulin-dependent, 20; Diamond-Blackfan anemia 1, 5, 8, and 10; Diarrhea 3 (secretory sodium, congenital, syndromic) and 5 (with tufting enteropathy, congenital); Dicarboxylic aminoaciduria; Diffuse palmoplantar keratoderma, Bothnian type; Digitorenocerebral syndrome; Dihydropteridine reductase deficiency; Dilated cardiomyopathy IA, IAA, 1C, 1G, 1BB, IDD, IFF, 1HH, 11, 1KK, IN, IS, 1Y, and 3B; Left ventricular noncompaction 3; Disordered steroidogenesis due to cytochrome p450 oxidoreductase deficiency; Distal arthrogryposis type 2B; Distal hereditary motor neuronopathy type 2B; Distal myopathy Markesbery-Griggs type; Distal spinal muscular atrophy, X-linked 3; Distichiasis-lymphedema syndrome; Dominant dystrophic epidermolysis bullosa with absence of skin; Dominant hereditary optic atrophy; Donnai Barrow syndrome; Dopamine beta hydroxylase deficiency; Dopamine receptor d2, reduced brain density of; Dowling-degos disease 4; Doyne honeycomb retinal dystrophy; Malattia leventinese; Duane syndrome type 2; Dubin-Johnson syndrome; Duchenne muscular dystrophy; Becker muscular dystrophy; Dysfibrinogenemia; Dyskeratosis congenita autosomal dominant and autosomal dominant, 3; Dyskeratosis congenita, autosomal recessive, 1, 3, 4, and 5; Dyskeratosis congenita X-linked; Dyskinesia, familial, with facial myokymia; Dysplasminogenemia; Dystonia 2 (torsion, autosomal recessive), 3 (torsion, X-linked), 5 (Dopa-responsive type), 10, 12, 16, 25, 26 (Myoclonic); Seizures, benign familial infantile, 2; Early infantile epileptic encephalopathy 2, 4, 7, 9, 10, 11, 13, and 14; Atypical Rett syndrome; Early T cell progenitor acute lymphoblastic leukemia; Ectodermal dysplasia skin fragility syndrome; Ectodermal dysplasia-syndactyly syndrome 1; Ectopia lentis, isolated autosomal recessive and dominant; Ectrodactyly, ectodermal dysplasia, and cleft lip/palate syndrome 3; Ehlers-Danlos syndrome type 7 (autosomal recessive), classic type, type 2 (progeroid), hydroxylysine-deficient, type 4, type 4 variant, and due to tenascin-X deficiency; Eichsfeld type congenital muscular dystrophy; Endocrine-cerebroosteodysplasia; Enhanced s-cone syndrome; Enlarged vestibular aqueduct syndrome; Enterokinase deficiency; Epidermodysplasia verruciformis; Epidermolysa bullosa simplex and limb girdle muscular dystrophy, simplex with mottled pigmentation, simplex with pyloric atresia, simplex, autosomal recessive, and with pyloric atresia; Epidermolytic palmoplantar keratoderma; Familial febrile seizures 8; Epilepsy, childhood absence 2, 12 (idiopathic generalized, susceptibility to) 5 (nocturnal frontal lobe), nocturnal frontal lobe type 1, partial, with variable foci, progressive myoclonic 3, and X-linked, with variable learning disabilities and behavior disorders; Epileptic encephalopathy, childhood-onset, early infantile, 1, 19, 23, 25, 30, and 32; Epiphyseal dysplasia, multiple, with myopia and conductive deafness; Episodic ataxia type 2; Episodic pain syndrome, familial, 3; Epstein syndrome; Fechtner syndrome; Erythropoietic protoporphyria; Estrogen resistance; Exudative vitreoretinopathy 6; Fabry disease and Fabry disease, cardiac variant; Factor H, VII, X, v and factor viii, combined deficiency of 2, xiii, a subunit, deficiency; Familial adenomatous polyposis 1 and 3; Familial amyloid nephropathy with urticaria and deafness; Familial cold urticarial; Familial aplasia of the vermis; Familial benign pemphigus; Familial cancer of breast; Breast cancer, susceptibility to; Osteosarcoma; Pancreatic cancer 3; Familial cardiomyopathy; Familial cold autoinflammatory syndrome 2; Familial colorectal cancer; Familial exudative vitreoretinopathy, X-linked; Familial hemiplegic migraine types 1 and 2; Familial hypercholesterolemia; Familial hypertrophic cardiomyopathy 1, 2, 3, 4, 7, 10, 23 and 24; Familial hypokalemia-hypomagnesemia; Familial hypoplastic, glomerulocystic kidney; Familial infantile myasthenia; Familial juvenile gout; Familial Mediterranean fever and Familial mediterranean fever, autosomal dominant; Familial porencephaly; Familial porphyria cutanea tarda; Familial pulmonary capillary hemangiomatosis; Familial renal glucosuria; Familial renal hypouricemia; Familial restrictive cardiomyopathy 1; Familial type 1 and 3 hyperlipoproteinemia; Fanconi anemia, complementation group E, I, N, and O; Fanconi-Bickel syndrome; Favism, susceptibility to; Febrile seizures, familial, 11; Feingold syndrome 1; Fetal hemoglobin quantitative trait locus 1; FG syndrome and FG syndrome 4; Fibrosis of extraocular muscles, congenital, 1, 2, 3a (with or without extraocular involvement), 3b; Fish-eye disease; Fleck corneal dystrophy; Floating-Harbor syndrome; Focal epilepsy with speech disorder with or without mental retardation; Focal segmental glomerulosclerosis 5; Forebrain defects; Frank Ter Haar syndrome; Borrone Di Rocco Crovato syndrome; Frasier syndrome; Wilms tumor 1; Freeman-Sheldon syndrome; Frontometaphyscal dysplasia land 3; Frontotemporal dementia; Frontotemporal dementia and/or amyotrophic lateral sclerosis 3 and 4; Frontotemporal Dementia Chromosome 3-Linked and Frontotemporal dementia ubiquitin-positive; Fructose-biphosphatase deficiency; Fuhrmann syndrome; Gamma-aminobutyric acid transaminase deficiency; Gamstorp-Wohlfart syndrome; Gaucher disease type 1 and Subacute neuronopathic; Gaze palsy, familial horizontal, with progressive scoliosis; Generalized dominant dystrophic epidermolysis bullosa; Generalized epilepsy with febrile seizures plus 3, type 1, type 2; Epileptic encephalopathy Lennox-Gastaut type; Giant axonal neuropathy; Glanzmann thrombasthenia; Glaucoma 1, open angle, e, F, and G; Glaucoma 3, primary congenital, d; Glaucoma, congenital and Glaucoma, congenital, Coloboma; Glaucoma, primary open angle, juvenile-onset; Glioma susceptibility 1; Glucose transporter type 1 deficiency syndrome; Glucose-6-phosphate transport defect; GLUTI deficiency syndrome 2; Epilepsy, idiopathic generalized, susceptibility to, 12; Glutamate formiminotransferase deficiency; Glutaric acidemia IIA and IIB; Glutaric aciduria, type 1; Gluthathione synthetase deficiency; Glycogen storage disease 0 (muscle), II (adult form), IXa2, IXc, type 1A; type II, type IV, IV (combined hepatic and myopathic), type V, and type VI; Goldmann-Favre syndrome; Gordon syndrome; Gorlin syndrome; Holoprosencephaly sequence; Holoprosencephaly 7; Granulomatous disease, chronic, X-linked, variant; Granulosa cell tumor of the ovary; Gray platelet syndrome; Griscelli syndrome type 3; Groenouw corneal dystrophy type I; Growth and mental retardation, mandibulofacial dysostosis, microcephaly, and cleft palate; Growth hormone deficiency with pituitary anomalies; Growth hormone insensitivity with immunodeficiency; GTP cyclohydrolase I deficiency; Hajdu-Cheney syndrome; Hand foot uterus syndrome; Hearing impairment; Hemangioma, capillary infantile; Hematologic neoplasm; Hemochromatosis type 1, 2B, and 3; Microvascular complications of diabetes 7; Transferrin serum level quantitative trait locus 2; Hemoglobin H disease, nondeletional; Hemolytic anemia, nonspherocytic, due to glucose phosphate isomerase deficiency; Hemophagocytic lymphohistiocytosis, familial, 2; Hemophagocytic lymphohistiocytosis, familial, 3; Heparin cofactor II deficiency; Hereditary acrodermatitis enteropathica; Hereditary breast and ovarian cancer syndrome; Ataxia-telangiectasia-like disorder; Hereditary diffuse gastric cancer; Hereditary diffuse leukoencephalopathy with spheroids; Hereditary factors II, IX, VIII deficiency disease; Hereditary hemorrhagic telangiectasia type 2; Hereditary insensitivity to pain with anhidrosis; Hereditary lymphedema type I; Hereditary motor and sensory neuropathy with optic atrophy; Hereditary myopathy with early respiratory failure; Hereditary neuralgic amyotrophy; Hereditary Nonpolyposis Colorectal Neoplasms; Lynch syndrome I and II; Hereditary pancreatitis; Pancreatitis, chronic, susceptibility to; Hereditary sensory and autonomic neuropathy type IIB amd IIA; Hereditary sideroblastic anemia; Hermansky-Pudlak syndrome 1, 3, 4, and 6; Heterotaxy, visceral, 2, 4, and 6, autosomal; Heterotaxy, visceral, X-linked; Heterotopia; Histiocytic medullary reticulosis; Histiocytosis-lymphadenopathy plus syndrome; Holocarboxylase synthetase deficiency; Holoprosencephaly 2, 3,7, and 9; Holt-Oram syndrome; Homocysteinemia due to MTHFR deficiency, CBS deficiency, and Homocystinuria, pyridoxine-responsive; Homocystinuria-Megaloblastic anemia due to defect in cobalamin metabolism, cblE complementation type; Howel-Evans syndrome; Hurler syndrome; Hutchinson-Gilford syndrome; Hydrocephalus; Hyperammonemia, type III; Hypercholesterolacmia and Hypercholesterolemia, autosomal recessive; Hyperekplexia 2 and Hyperekplexia hereditary; Hyperferritinemia cataract syndrome; Hyperglycinuria; Hyperimmunoglobulin D with periodic fever; Mevalonic aciduria; Hyperimmunoglobulin E syndrome; Hyperinsulinemic hypoglycemia familial 3, 4, and 5; Hyperinsulinism-hyperammonemia syndrome; Hyperlysinemia; Hypermanganesemia with dystonia, polycythemia and cirrhosis; Hyperornithinemia-hyperammonemia-homocitrullinuria syndrome; Hyperparathyroidism 1 and 2; Hyperparathyroidism, neonatal severe; Hyperphenylalaninemia, bh4-deficient, a, due to partial pts deficiency, BH4-deficient, D, and non-pku; Hyperphosphatasia with mental retardation syndrome 2, 3, and 4; Hypertrichotic osteochondrodysplasia; Hypobetalipoproteinemia, familial, associated with apob32; Hypocalcemia, autosomal dominant 1; Hypocalciuric hypercalcemia, familial, types 1 and 3; Hypochondrogenesis; Hypochromic microcytic anemia with iron overload; Hypoglycemia with deficiency of glycogen synthetase in the liver; Hypogonadotropic hypogonadism 11 with or without anosmia; Hypohidrotic ectodermal dysplasia with immune deficiency; Hypohidrotic X-linked ectodermal dysplasia; Hypokalemic periodic paralysis 1 and 2; Hypomagnesemia 1, intestinal; Hypomagnesemia, seizures, and mental retardation; Hypomyelinating leukodystrophy 7; Hypoplastic left heart syndrome; Atrioventricular septal defect and common atrioventricular junction; Hypospadias 1 and 2, X-linked; Hypothyroidism, congenital, nongoitrous, 1; Hypotrichosis 8 and 12; Hypotrichosis-lymphedema-telangiectasia syndrome; I blood group system; Ichthyosis bullosa of Siemens; Ichthyosis exfoliativa; Ichthyosis prematurity syndrome; Idiopathic basal ganglia calcification 5; Idiopathic fibrosing alveolitis, chronic form; Dyskeratosis congenita, autosomal dominant, 2 and 5; Idiopathic hypercalcemia of infancy; Immune dysfunction with T-cell inactivation due to calcium entry defect 2; Immunodeficiency 15, 16, 19, 30, 31C, 38, 40, 8, due to defect in cd3-zeta, with hyper IgM type 1 and 2, and X-Linked, with magnesium defect, Epstein-Barr virus infection, and neoplasia; Immunodeficiency-centromeric instability-facial anomalies syndrome 2; Inclusion body myopathy 2 and 3; Nonaka myopathy; Infantile convulsions and paroxysmal chorcoathetosis, familial; Infantile cortical hyperostosis; Infantile GM1 gangliosidosis; Infantile hypophosphatasia; Infantile nephronophthisis; Infantile nystagmus, X-linked; Infantile Parkinsonism-dystonia; Infertility associated with multi-tailed spermatozoa and excessive DNA; Insulin resistance; Insulin-resistant diabetes mellitus and acanthosis nigricans; Insulin-dependent diabetes mellitus secretory diarrhea syndrome; Interstitial nephritis, karyomegalic; Intrauterine growth retardation, metaphyscal dysplasia, adrenal hypoplasia congenita, and genital anomalies; lodotyrosyl coupling defect; IRAK4 deficiency; Iridogoniodysgenesis dominant type and type 1; Iron accumulation in brain; Ischiopatellar dysplasia; Islet cell hyperplasia; Isolated 17,20-lyase deficiency; Isolated lutropin deficiency; Isovaleryl-CoA dehydrogenase deficiency; Jankovic Rivera syndrome; Jervell and Lange-Nielsen syndrome 2; Joubert syndrome 1, 6, 7, 9/15 (digenic), 14, 16, and 17, and Orofaciodigital syndrome xiv; Junctional epidermolysis bullosa gravis of Herlitz; Juvenile GM>1<gangliosidosis; Juvenile polyposis syndrome; Juvenile polyposis/hereditary hemorrhagic telangiectasia syndrome; Juvenile retinoschisis; Kabuki make-up syndrome; Kallmann syndrome 1, 2, and 6; Delayed puberty; Kanzaki disease; Karak syndrome; Kartagener syndrome; Kenny-Caffey syndrome type 2; Keppen-Lubinsky syndrome; Keratoconus 1; Keratosis follicularis; Keratosis palmoplantaris striata 1; Kindler syndrome; L-2-hydroxyglutaric aciduria; Larsen syndrome, dominant type; Lattice corneal dystrophy Type III; Leber amaurosis; Zellweger syndrome; Peroxisome biogenesis disorders; Zellweger syndrome spectrum; Leber congenital amaurosis 11, 12, 13, 16, 4, 7, and 9; Leber optic atrophy; Aminoglycoside-induced deafness; Deafness, nonsyndromic sensorineural, mitochondrial; Left ventricular noncompaction 5; Left-right axis malformations; Leigh disease; Mitochondrial short-chain Enoyl-CoA Hydratase 1 deficiency; Leigh syndrome due to mitochondrial complex I deficiency; Leiner disease; Leri Weill dyschondrosteosis; Lethal congenital contracture syndrome 6; Leukocyte adhesion deficiency type I and III; Leukodystrophy, Hypomyelinating, 11 and 6; Leukoencephalopathy with ataxia, with Brainstem and Spinal Cord Involvement and Lactate Elevation, with vanishing white matter, and progressive, with ovarian failure; Leukonychia totalis; Lewy body dementia; Lichtenstein-Knorr Syndrome; Li-Fraumeni syndrome 1; Lig4 syndrome; Limb-girdle muscular dystrophy, type 1B, 2A, 2B, 2D, C1, C5, C9, C14; Congenital muscular dystrophy-dystroglycanopathy with brain and eye anomalies, type A14 and B14; Lipase deficiency combined; Lipid protcinosis; Lipodystrophy, familial partial, type 2 and 3; Lissencephaly 1, 2 (X-linked), 3, 6 (with microcephaly), X-linked; Subcortical laminar heterotopia, X-linked; Liver failure acute infantile; Locys-Dietz syndrome 1, 2, 3; Long QT syndrome 1, 2, 2/9, 2/5, (digenic), 3, 5 and 5, acquired, susceptibility to; Lung cancer; Lymphedema, hereditary, id; Lymphedema, primary, with myelodysplasia; Lymphoproliferative syndrome 1, 1 (X-linked), and 2; Lysosomal acid lipase deficiency; Macrocephaly, macrosomia, facial dysmorphism syndrome; Macular dystrophy, vitelliform, adult-onset; Malignant hyperthermia susceptibility type 1; Malignant lymphoma, non-Hodgkin; Malignant melanoma; Malignant tumor of prostate; Mandibuloacral dysostosis; Mandibuloacral dysplasia with type A or B lipodystrophy, atypical; Mandibulofacial dysostosis, Treacher Collins type, autosomal recessive; Mannose-binding protein deficiency; Maple syrup urine disease type 1A and type 3; Marden Walker like syndrome; Marfan syndrome; Marinesco-Sj\xc3\xb6gren syndrome; Martsolf syndrome; Maturity-onset diabetes of the young, type 1, type 2, type 11, type 3, and type 9; May-Hegglin anomaly; MYH9 related disorders; Sebastian syndrome; McCune-Albright syndrome; Somatotroph adenoma; Sex cord-stromal tumor; Cushing syndrome; McKusick Kaufman syndrome; McLeod neuroacanthocytosis syndrome; Meckel-Gruber syndrome; Medium-chain acyl-coenzyme A dehydrogenase deficiency; Medulloblastoma; Megalencephalic leukoencephalopathy with subcortical cysts land 2a; Megalencephaly cutis marmorata telangiectatica congenital; PIK3CA Related Overgrowth Spectrum; Megalencephaly-polymicrogyria-polydactyly-hydrocephalus syndrome 2; Megaloblastic anemia, thiamine-responsive, with diabetes mellitus and sensorineural deafness; Meier-Gorlin syndromes land 4; Melnick-Needles syndrome; Meningioma; Mental retardation, X-linked, 3, 21, 30, and 72; Mental retardation and microcephaly with pontine and cerebellar hypoplasia; Mental retardation X-linked syndromic 5; Mental retardation, anterior maxillary protrusion, and strabismus; Mental retardation, autosomal dominant 12, 13, 15, 24, 3, 30, 4, 5, 6, and 9; Mental retardation, autosomal recessive 15, 44, 46, and 5; Mental retardation, stereotypic movements, epilepsy, and/or cerebral malformations; Mental retardation, syndromic, Claes-Jensen type, X-linked; Mental retardation, X-linked, nonspecific, syndromic, Hedera type, and syndromic, wu type; Merosin deficient congenital muscular dystrophy; Metachromatic leukodystrophy juvenile, late infantile, and adult types; Metachromatic leukodystrophy; Metatrophic dysplasia; Methemoglobinemia types I and 2; Methionine adenosyltransferase deficiency, autosomal dominant; Methylmalonic acidemia with homocystinuria; Methylmalonic aciduria cblB type; Methylmalonic aciduria due to methylmalonyl-CoA mutase deficiency; METHYLMALONIC ACIDURIA, mut (0) TYPE; Microcephalic osteodysplastic primordial dwarfism type 2; Microcephaly with or without chorioretinopathy, lymphedema, or mental retardation; Microcephaly, hiatal hernia and nephrotic syndrome; Microcephaly; Hypoplasia of the corpus callosum; Spastic paraplegia 50, autosomal recessive; Global developmental delay; CNS hypomyelination; Brain atrophy; Microcephaly, normal intelligence and immunodeficiency; Microcephaly-capillary malformation syndrome; Microcytic anemia; Microphthalmia syndromic 5, 7, and 9; Microphthalmia, isolated 3, 5, 6, 8, and with coloboma 6; Microspherophakia; Migraine, familial basilar; Miller syndrome; Minicore myopathy with external ophthalmoplegia; Myopathy, congenital with cores; Mitchell-Riley syndrome; mitochondrial 3-hydroxy-3-methylglutaryl-CoA synthase deficiency; Mitochondrial complex I, II, III, III (nuclear type 2, 4, or 8) deficiency; Mitochondrial DNA depletion syndrome 11, 12 (cardiomyopathie type), 2, 4B (MNGIE type), 8B (MNGIE type); Mitochondrial DNA-depletion syndrome 3 and 7, hepatocerebral types, and 13 (encephalomyopathic type); Mitochondrial phosphate carrier and pyruvate carrier deficiency; Mitochondrial trifunctional protein deficiency; Long-chain 3-hydroxyacyl-CoA dehydrogenase deficiency; Miyoshi muscular dystrophy 1; Myopathy, distal, with anterior tibial onset; Mohr-Tranebjaerg syndrome; Molybdenum cofactor deficiency, complementation group A; Mowat-Wilson syndrome; Mucolipidosis III Gamma; Mucopolysaccharidosis type VI, type VI (severe), and type VII; Mucopolysaccharidosis, MPS—I-H/S, MPS-II, MPS-III-A, MPS—III—B, MPS—III—C, MPS-IV-A, MPS—IV-B; Retinitis Pigmentosa 73; Gangliosidosis GM1 typel (with cardiac involvenment) 3; Multicentric osteolysis nephropathy; Multicentric osteolysis, nodulosis and arthropathy; Multiple congenital anomalies; Atrial septal defect 2; Multiple congenital anomalies-hypotonia-seizures syndrome 3; Multiple Cutaneous and Mucosal Venous Malformations; Multiple endocrine neoplasia, types land 4; Multiple epiphyseal dysplasia 5 or Dominant; Multiple gastrointestinal atresias; Multiple pterygium syndrome Escobar type; Multiple sulfatase deficiency; Multiple synostoses syndrome 3; Muscle AMP deaminase deficiency; Muscle eye brain disease; Muscular dystrophy, congenital, megaconial type; Myasthenia, familial infantile, 1; Myasthenic Syndrome, Congenital, 11, associated with acetylcholine receptor deficiency; Myasthenic Syndrome, Congenital, 17, 2A (slow-channel), 4B (fast-channel), and without tubular aggregates; Myeloperoxidase deficiency; MYH-associated polyposis; Endometrial carcinoma; Myocardial infarction 1; Myoclonic dystonia; Myoclonic-Atonic Epilepsy; Myoclonus with epilepsy with ragged red fibers; Myofibrillar myopathy 1 and ZASP-related; Myoglobinuria, acute recurrent, autosomal recessive; Myoneural gastrointestinal encephalopathy syndrome; Cerebellar ataxia infantile with progressive external ophthalmoplegia; Mitochondrial DNA depletion syndrome 4B, MNGIE type; Myopathy, centronuclear, 1, congenital, with excess of muscle spindles, distal, 1, lactic acidosis, and sideroblastic anemia 1, mitochondrial progressive with congenital cataract, hearing loss, and developmental delay, and tubular aggregate, 2; Myopia 6; Myosclerosis, autosomal recessive; Myotonia congenital; Congenital myotonia, autosomal dominant and recessive forms; Nail-patella syndrome; Nance-Horan syndrome; Nanophthalmos 2; Navajo neurohepatopathy; Nemaline myopathy 3 and 9; Neonatal hypotonia; Intellectual disability; Seizures; Delayed speech and language development; Mental retardation, autosomal dominant 31; Neonatal intrahepatic cholestasis caused by citrin deficiency; Nephrogenic diabetes insipidus, Nephrogenic diabetes insipidus, X-linked; Nephrolithiasis/osteoporosis, hypophosphatemic, 2; Nephronophthisis 13, 15 and 4; Infertility; Cerebello-oculo-renal syndrome (nephronophthisis, oculomotor apraxia and cerebellar abnormalities); Nephrotic syndrome, type 3, type 5, with or without ocular abnormalities, type 7, and type 9; Nestor-Guillermo progeria syndrome; Neu-Laxova syndrome 1; Neurodegeneration with brain iron accumulation 4 and 6; Neuroferritinopathy; Neurofibromatosis, type land type 2; Neurofibrosarcoma; Neurohypophyseal diabetes insipidus; Neuropathy, Hereditary Sensory, Type 1C; Neutral 1 amino acid transport defect; Neutral lipid storage disease with myopathy; Neutrophil immunodeficiency syndrome; Nicolaides-Baraitser syndrome; Niemann-Pick disease type C1, C2, type A, and type C1, adult form; Non-ketotic hyperglycinemia; Noonan syndrome 1 and 4, LEOPARD syndrome 1; Noonan syndrome-like disorder with or without juvenile myelomonocytic leukemia; Normokalemic periodic paralysis, potassium-sensitive; Norum disease; Epilepsy, Hearing Loss, And Mental Retardation Syndrome; Mental Retardation, X-Linked 102 and syndromic 13; Obesity; Ocular albinism, type I; Oculocutaneous albinism type 1B, type 3, and type 4; Oculodentodigital dysplasia; Odontohypophosphatasia; Odontotrichomelic syndrome; Oguchi disease; Oligodontia-colorectal cancer syndrome; Opitz G/BBB syndrome; Optic atrophy 9; Oral-facial-digital syndrome; Ornithine aminotransferase deficiency; Orofacial cleft 11 and 7, Cleft lip/palate-ectodermal dysplasia syndrome; Orstavik Lindemann Solberg syndrome; Osteoarthritis with mild chondrodysplasia; Osteochondritis dissecans; Osteogenesis imperfecta type 12, type 5, type 7, type 8, type I, type III, with normal sclerae, dominant form, recessive perinatal lethal; Osteopathia striata with cranial sclerosis; Osteopetrosis autosomal dominant type 1 and 2, recessive 4, recessive 1, recessive 6; Osteoporosis with pseudoglioma; Oto-palato-digital syndrome, types I and II; Ovarian dysgenesis 1; Ovarioleukodystrophy; Pachyonychia congenita 4 and type 2; Paget disease of bone, familial; Pallister-Hall syndrome; Palmoplantar keratoderma, nonepidermolytic, focal or diffuse; Pancreatic agenesis and congenital heart disease; Papillon-Lef\xc3\xa8vre syndrome; Paragangliomas 3; Paramyotonia congenita of von Eulenburg; Parathyroid carcinoma; Parkinson disease 14, 15, 19 (juvenile-onset), 2, 20 (early-onset), 6, (autosomal recessive early-onset, and 9; Partial albinism; Partial hypoxanthine-guanine phosphoribosyltransferase deficiency; Patterned dystrophy of retinal pigment epithelium; PC-K6a; Pelizacus-Merzbacher disease; Pendred syndrome; Peripheral demyelinating neuropathy, central dysmyelination; Hirschsprung disease; Permanent neonatal diabetes mellitus; Diabetes mellitus, permanent neonatal, with neurologic features; Neonatal insulin-dependent diabetes mellitus; Maturity-onset diabetes of the young, type 2; Peroxisome biogenesis disorder 14B, 2A, 4A, 5B, 6A, 7A, and 7B; Perrault syndrome 4; Perry syndrome; Persistent hyperinsulinemic hypoglycemia of infancy; familial hyperinsulinism; Phenotypes; Phenylketonuria; Pheochromocytoma; Hereditary Paraganglioma-Pheochromocytoma Syndromes; Paragangliomas 1; Carcinoid tumor of intestine; Cowden syndrome 3; Phosphoglycerate dehydrogenase deficiency; Phosphoglycerate kinase 1 deficiency; Photosensitive trichothiodystrophy; Phytanic acid storage disease; Pick disease; Pierson syndrome; Pigmentary retinal dystrophy; Pigmented nodular adrenocortical disease, primary, 1; Pilomatrixoma; Pitt-Hopkins syndrome; Pituitary dependent hypercortisolism; Pituitary hormone deficiency, combined 1, 2, 3, and 4; Plasminogen activator inhibitor type 1 deficiency; Plasminogen deficiency, type I; Platelet-type bleeding disorder 15 and 8; Poikiloderma, hereditary fibrosing, with tendon contractures, myopathy, and pulmonary fibrosis; Polycystic kidney disease 2, adult type, and infantile type; Polycystic lipomembranous osteodysplasia with sclerosing leukoencephalopathy; Polyglucosan body myopathy 1 with or without immunodeficiency; Polymicrogyria, asymmetric, bilateral frontoparietal; Polyneuropathy, hearing loss, ataxia, retinitis pigmentosa, and cataract; Pontocerebellar hypoplasia type 4; Popliteal pterygium syndrome; Porencephaly 2; Porokeratosis 8, disseminated superficial actinic type; Porphobilinogen synthase deficiency; Porphyria cutanea tarda; Posterior column ataxia with retinitis pigmentosa; Posterior polar cataract type 2; Prader-Willi-like syndrome; Premature ovarian failure 4, 5, 7, and 9; Primary autosomal recessive microcephaly 10, 2, 3, and 5; Primary ciliary dyskinesia 24; Primary dilated cardiomyopathy; Left ventricular noncompaction 6; 4, Left ventricular noncompaction 10; Paroxysmal atrial fibrillation; Primary hyperoxaluria, type I, type, and type III; Primary hypertrophic osteoarthropathy, autosomal recessive 2; Primary hypomagnesemia; Primary open angle glaucoma juvenile onset 1; Primary pulmonary hypertension; Primrose syndrome; Progressive familial heart block type 1B; Progressive familial intrahepatic cholestasis 2 and 3; Progressive intrahepatic cholestasis; Progressive myoclonus epilepsy with ataxia; Progressive pseudorheumatoid dysplasia; Progressive sclerosing poliodystrophy; Prolidase deficiency; Proline dehydrogenase deficiency; Schizophrenia 4; Properdin deficiency, X-linked; Propionic academia; Proprotein convertase 1/3 deficiency; Prostate cancer, hereditary, 2; Protan defect; Proteinuria; Finnish congenital nephrotic syndrome; Proteus syndrome; Breast adenocarcinoma; Pseudoachondroplastic spondyloepiphyseal dysplasia syndrome; Pseudohypoaldosteronism type 1 autosomal dominant and recessive and type 2; Pseudohypoparathyroidism type 1A, Pseudopseudohypoparathyroidism; Pseudonconatal adrenoleukodystrophy; Pseudoprimary hyperaldosteronism; Pseudoxanthoma elasticum; Generalized arterial calcification of infancy 2; Pseudoxanthoma elasticum-like disorder with multiple coagulation factor deficiency; Psoriasis susceptibility 2; PTEN hamartoma tumor syndrome; Pulmonary arterial hypertension related to hereditary hemorrhagic telangiectasia; Pulmonary Fibrosis And/Or Bone Marrow Failure, Telomere-Related, 1 and 3; Pulmonary hypertension, primary, 1, with hereditary hemorrhagic telangiectasia; Purine-nucleoside phosphorylase deficiency; Pyruvate carboxylase deficiency; Pyruvate dehydrogenase El-alpha deficiency; Pyruvate kinase deficiency of red cells; Raine syndrome; Rasopathy; Recessive dystrophic epidermolysis bullosa; Nail disorder, nonsyndromic congenital, 8; Reifenstein syndrome; Renal adysplasia; Renal carnitine transport defect; Renal coloboma syndrome; Renal dysplasia; Renal dysplasia, retinal pigmentary dystrophy, cerebellar ataxia and skeletal dysplasia; Renal tubular acidosis, distal, autosomal recessive, with late-onset sensorineural hearing loss, or with hemolytic anemia; Renal tubular acidosis, proximal, with ocular abnormalities and mental retardation; Retinal cone dystrophy 3B; Retinitis pigmentosa; Retinitis pigmentosa 10, 11, 12, 14, 15, 17, and 19; Retinitis pigmentosa 2, 20, 25, 35, 36, 38, 39, 4, 40, 43, 45, 48, 66, 7, 70, 72; Retinoblastoma; Rett disorder; Rhabdoid tumor predisposition syndrome 2; Rhegmatogenous retinal detachment, autosomal dominant; Rhizomelic chondrodysplasia punctata type 2 and type 3; Roberts-SC phocomelia syndrome; Robinow Sorauf syndrome; Robinow syndrome, autosomal recessive, autosomal recessive, with brachy-syn-polydactyly; Rothmund-Thomson syndrome; Rapadilino syndrome; RRM2B-related mitochondrial disease; Rubinstein-Taybi syndrome; Salla disease; Sandhoff disease, adult and infantil types; Sarcoidosis, early-onset; Blau syndrome; Schindler disease, type 1; Schizencephaly; Schizophrenia 15; Schneckenbecken dysplasia; Schwannomatosis 2; Schwartz Jampel syndrome type 1; Sclerocornea, autosomal recessive; Sclerosteosis; Secondary hypothyroidism; Segawa syndrome, autosomal recessive; Senior-Loken syndrome 4 and 5; Sensory ataxic neuropathy, dysarthria, and ophthalmoparesis; Sepiapterin reductase deficiency; SeSAME syndrome; Severe combined immunodeficiency due to ADA deficiency, with microcephaly, growth retardation, and sensitivity to ionizing radiation, atypical, autosomal recessive, T cell-negative, B cell-positive, NK cell-negative of NK-positive; Partial adenosine deaminase deficiency; Severe congenital neutropenia; Severe congenital neutropenia 3, autosomal recessive or dominant; Severe congenital neutropenia and 6, autosomal recessive; Severe myoclonic epilepsy in infancy; Generalized epilepsy with febrile seizures plus, types 1 and 2; Severe X-linked myotubular myopathy; Short QT syndrome 3; Short stature with nonspecific skeletal abnormalities; Short stature, auditory canal atresia, mandibular hypoplasia, skeletal abnormalities; Short stature, onychodysplasia, facial dysmorphism, and hypotrichosis; Primordial dwarfism; Short-rib thoracic dysplasia 11 or 3 with or without polydactyly; Sialidosis type I and II; Silver spastic paraplegia syndrome; Slowed nerve conduction velocity, autosomal dominant; Smith-Lemli-Opitz syndrome; Snyder Robinson syndrome; Somatotroph adenoma; Prolactinoma; familial, Pituitary adenoma predisposition; Sotos syndrome 1 or 2; Spastic ataxia 5, autosomal recessive, Charlevoix-Saguenay type, 1,10, or 11, autosomal recessive; Amyotrophic lateral sclerosis type 5; Spastic paraplegia 15, 2, 3, 35, 39, 4, autosomal dominant, 55, autosomal recessive, and 5A; Bile acid synthesis defect, congenital, 3; Spermatogenic failure 11, 3, and 8; Spherocytosis types 4 and 5; Spheroid body myopathy; Spinal muscular atrophy, lower extremity predominant 2, autosomal dominant; Spinal muscular atrophy, type II; Spinocerebellar ataxia 14, 21, 35, 40, and 6; Spinocerebellar ataxia autosomal recessive 1 and 16; Splenic hypoplasia; Spondylocarpotarsal synostosis syndrome; Spondylocheirodysplasia, Ehlers-Danlos syndrome-like, with immune dysregulation, Aggrecan type, with congenital joint dislocations, short limb-hand type, Sedaghatian type, with conc-rod dystrophy, and Kozlowski type; Parastremmatic dwarfism; Stargardt disease 1; Cone-rod dystrophy 3; Stickler syndrome type 1; Kniest dysplasia; Stickler syndrome, types 1 (nonsyndromic ocular) and 4; Sting-associated vasculopathy, infantile-onset; Stormorken syndrome; Sturge-Weber syndrome, Capillary malformations, congenital, 1; Succinyl-CoA acetoacetate transferase deficiency; Sucrase-isomaltase deficiency; Sudden infant death syndrome; Sulfite oxidase deficiency, isolated; Supravalvar aortic stenosis; Surfactant metabolism dysfunction, pulmonary, 2 and 3; Symphalangism, proximal, 1b; Syndactyly Cenani Lenz type; Syndactyly type 3; Syndromic X-linked mental retardation 16; Talipes equinovarus; Tangier disease; TARP syndrome; Tay-Sachs disease, B1 variant, Gm2-gangliosidosis (adult), Gm2-gangliosidosis (adult-onset); Temtamy syndrome; Tenorio Syndrome; Terminal osseous dysplasia; Testosterone 17-beta-dehydrogenase deficiency; Tetraamelia, autosomal recessive; Tetralogy of Fallot; Hypoplastic left heart syndrome 2; Truncus arteriosus; Malformation of the heart and great vessels; Ventricular septal defect 1; Thiel-Behnke corneal dystrophy; Thoracic aortic aneurysms and aortic dissections; Marfanoid habitus; Three M syndrome 2; Thrombocytopenia, platelet dysfunction, hemolysis, and imbalanced globin synthesis; Thrombocytopenia, X-linked; Thrombophilia, hereditary, due to protein C deficiency, autosomal dominant and recessive; Thyroid agenesis; Thyroid cancer, follicular; Thyroid hormone metabolism, abnormal; Thyroid hormone resistance, generalized, autosomal dominant; Thyrotoxic periodic paralysis and Thyrotoxic periodic paralysis 2; Thyrotropin-releasing hormone resistance, generalized; Timothy syndrome; TNF receptor-associated periodic fever syndrome (TRAPS); Tooth agenesis, selective, 3 and 4; Torsades de pointes; Townes-Brocks-branchiootorenal-like syndrome; Transient bullous dermolysis of the newborn; Treacher collins syndrome 1; Trichomegaly with mental retardation, dwarfism and pigmentary degeneration of retina; Trichorhinophalangeal dysplasia type I; Trichorhinophalangcal syndrome type 3; Trimethylaminuria; Tuberous sclerosis syndrome; Lymphangiomyomatosis; Tuberous sclerosis 1 and 2; Tyrosinase-negative oculocutaneous albinism; Tyrosinase-positive oculocutaneous albinism; Tyrosinemia type I; UDPglucose-4-epimerase deficiency; Ullrich congenital muscular dystrophy; Ulna and fibula absence of with severe limb deficiency; Upshaw-Schulman syndrome; Urocanate hydratase deficiency; Usher syndrome, types 1, 1B, ID, 1G, 2A, 2C, and 2D; Retinitis pigmentosa 39; UV-sensitive syndrome; Van der Woude syndrome; Van Maldergem syndrome 2; Hennekam lymphangiectasia-lymphedema syndrome 2; Variegate porphyria; Ventriculomegaly with cystic kidney disease; Verheij syndrome; Very long chain acyl-CoA dehydrogenase deficiency; Vesicoureteral reflux 8; Visceral heterotaxy 5, autosomal; Visceral myopathy; Vitamin D-dependent rickets, types land 2; Vitelliform dystrophy; von Willebrand disease type 2M and type 3; Waardenburg syndrome type 1, 4C, and 2E (with neurologic involvement); Klein-Waardenberg syndrome; Walker-Warburg congenital muscular dystrophy; Warburg micro syndrome 2 and 4; Warts, hypogammaglobulinemia, infections, and myelokathexis; Weaver syndrome; Weill-Marchesani syndrome 1 and 3; Weill-Marchesani-like syndrome; Weissenbacher-Zweymuller syndrome; Werdnig-Hoffmann disease; Charcot-Marie-Tooth disease; Werner syndrome; WFS1-Related Disorders; Wiedemann-Steiner syndrome; Wilson disease; Wolfram-like syndrome, autosomal dominant; Worth disease; Van Buchem disease type 2; Xeroderma pigmentosum, complementation group b, group D, group E, and group G; X-linked agammaglobulinemia; X-linked hereditary motor and sensory neuropathy; X-linked ichthyosis with steryl-sulfatase deficiency; X-linked periventricular heterotopia; Oto-palato-digital syndrome, type I; X-linked severe combined immunodeficiency; Zimmermann-Laband syndrome and Zimmermann-Laband syndrome 2; and Zonular pulverulent cataract 3.

The instant disclosure provides lists of genes comprising pathogenic G to A or C to T mutations. Such pathogenic G to A or C to T mutations may be corrected using the methods and compositions provided herein, for example by mutating the A to a G, and/or the T to a C, thereby restoring gene function. Table 2 includes exemplary mutations that can be corrected using base editors described herein. Table 2 includes the gene symbol, the associated phenotype, the mutation to be corrected and exemplary gRNA sequences which may be used to correct the mutations. The gRNA sequences provided in Table 2 are sequences that encode RNA that can direct Cas9, or any of the base editors provided herin, to a target site. For example, the gRNA sequences provided in Table 2 may be cloned into a gRNA expression vector, such as pFYF to encode a gRNA that targets Cas9, or any of the base editors provided herein, to a target site in order to correct a disease-related mutation. It should be appreciated, however, that additional mutations may be corrected to treat additional diseases associated with a G to A or C to T mutation. Furthermore, additional gRNAs may be designed based on the disclosure and the knowledge in the art, which would be appreciated by the skilled artisan.

### Pharmaceutical Compositions

Other aspects of the present disclosure relate to pharmaceutical compositions comprising any of the adenosine deaminases, fusion proteins, or the fusion protein-gRNA complexes described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid diolcoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-diolcoyloxi) propyl]-N,N,N-trimethyl-amoniummethylsulfate, or “DOTAP,” are particularly preferred for such particles and vesicles. The preparation of such lipid particles is well known. Sec, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### Delivery Methods

In some aspects, the invention provides methods comprising delivering one or more polynucleotides, such as or one or more vectors as described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and w2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

### Kits, Vectors, Cells

Some aspects of this disclosure provide kits comprising a nucleic acid construct comprising a nucleotide sequence encoding an adenosine deaminase capable of deaminating an adenosine in a deoxyribonucleic acid (DNA) molecule. In some embodiments, the nucleotide sequence encodes any of the adenosine deaminases provided herein. In some embodiments, the nucleotide sequence comprises a heterologous promoter that drives expression of the adenosine deaminase.

Some aspects of this disclosure provide kits comprising a nucleic acid construct, comprising (a) a nucleotide sequence encoding a napDNAbp (e.g., a Cas9 domain) fused to an adenosine deaminase, or a fusion protein comprising a napDNAbp (e.g., Cas9 domain) and an adenosine deaminase as provided herein; and (b) a heterologous promoter that drives expression of the sequence of (a). In some embodiments, the kit further comprises an expression construct encoding a guide nucleic acid backbone, (e.g., a guide RNA backbone), wherein the construct comprises a cloning site positioned to allow the cloning of a nucleic acid sequence identical or complementary to a target sequence into the guide nucleic acid (e.g., guide RNA backbone).

Some aspects of this disclosure provide cells comprising any of the adenosine deaminases, fusion proteins, or complexes provided herein. In some embodiments, the cells comprise a nucleotide that encodes any of the adenosine deaminases or fusion proteins provided herein. In some embodiments, the cells comprise any of the nucleotides or vectors provided herein.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh 1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO—IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/ARI, EMT6/AR10.0, FM3, H1299, H69, HB54, HB55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THPI cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

The description of exemplary embodiments of the reporter systems above is provided for illustration purposes only and not meant to be limiting. Additional reporter systems, e.g., variations of the exemplary systems described in detail above, are also embraced by this disclosure.

It should be appreciated however, that additional fusion proteins would be apparent to the skilled artisan based on the present disclosure and knowledge in the art.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### EXAMPLES

In order that the invention described herein may be more fully understood, the following examples are set forth. It should be understood that these examples are for illustrative purposes only and are not to be construed as limiting this invention in any manner.

### Example 1

Base editors, catalytically impaired Cas9 proteins fused to nucleobase modification enzymes, enable targeted single-nucleotide conversion in the genomes of a wide range of cells and organisms without inducing double-stranded DNA breaks. Previously, both C·G-to-T·A base editors (BE3 and BE4) and A·T-to-G·C base editors (ABE) were developed, and their targeting scope, product purity, and DNA specificity were enhanced. The usefulness of base editors for research and therapeutic applications is also strongly dependent on the efficiency with which they can modify target nucleotides. Here, factors that limit base editing efficiency in mammalian cells were examined and it was determined that the expression level and nuclear localization of current-generation BE4 and ABE base editors limit their editing efficiency. By improving the nuclear localization signal (NLS), optimizing the codon usage of base editor constructs, and performing ancestral reconstruction of the component deaminase domains, the efficiency of BE4- and ABE-mediated genome editing was greatly improved, in most tested cases to ˜80% target base conversion in HEK293T cells. It is shown that these optimized “BE4max”, “AncBE4max”, and “ABEmax” base editors are especially enabling under unfavorable conditions such as when delivery of constructs is limiting. These optimized editors corrected several pathogenic SNPs in a variety of mammalian cell types with substantially higher efficiencies than BE4 and ABE. AncBE4max, BE4max, and ABEmax substantially expand the capabilities of both cytidine and adenosine base editing and represent current state-of-the-art mammalian cell base editors.

Point mutations represent the majority of known human genetic variants associated with disease¹. Developing robust methods to introduce and correct point mutations is therefore an important challenge to understand and treat diseases with a genetic component. Base editors, fusions of catalytically disabled Cas9, natural or evolved nucleobase deaminases, and, in some cases, proteins have been recently developed, that alter cellular DNA repair processes to increase the efficiency and stability of the resulting single-nucleotide change^{2.3}. Two classes of base editors have been described to date: cytidine base editors convert target C·G base pairs to T·A, and adenosine base editors convert A·T to G·C. Collectively, these two classes of base editors enable the targeted installation of all four transition mutations (C-to-T, G-to-A, A-to-G, and T-to-C), which collectively account for 61% of known human pathogenic SNPs in the Clin Var database (FIGS. 1A, 1B). Base editors have been used widely in organisms ranging from prokaryotes to plants to amphibians to mammals, and have even been used to correct pathogenic mutations in human embryos⁴⁻¹⁸.

The utility of base editing is limited by several constraints, including the PAM requirement imposed by the Cas9 moiety, off-target base editing, “bystander editing” of non-target Cs or As very close to the target nucleotides, the production of undesired byproducts, and overall editing efficiency. Next-generation base editors have been developed that address some of these limitations, including base editors with different or expanded PAM compatibilities¹⁹⁻²¹, high-fidelity base editors with reduced off-target activity^{20, 22-25}, base editors with narrowed editing windows (normally ˜5 nucleotides wide)¹⁹, and a current-generation cytidine base editor (BE4) with greatly reduced byproducts⁶.

Despite these recent advances, the efficiency of base editing by BE4 and current adenine base editor (ABE) variants varies widely by cell type and target locus. To broadly increase base editing efficiency, it was sought to identify the factors that limit base editing efficiency in mammalian cells. In this study it was found that for both BE4 and ABE, expression and nuclear localization in human cells impose key bottlenecks on editing efficiency. Optimizing codon usage, using improved nuclear localization sequences (NLSs), and performing ancestral reconstruction of cytidine deaminases result in base editors with greatly increased editing efficiency, often more than doubling target nucleotide conversion yields. The resulting AncBE4max, BE4max, and ABEmax base editors install point mutations relevant to human disease in a variety of mammalian cell types much more efficiently than previously described base editors. AncBE4max, BE4max, and ABEmax substantially advance the utility of both classes of base editors, and their use is recommended for general base editing applications in mammalian cells.

### Results

Several factors could limit the ability of a base editor to achieve conversion of the target nucleotide. If the base editor is delivered as encoded DNA or RNA, cellular uptake of the nucleic acid can be limiting. Among cells that have acquired a DNA construct encoding a base editor, transcription may be limited by the choice of promoter or regulatory sequences or by the editor's coding sequence. Once transcribed, translation efficiency may also be limiting depending on factors including codon usage and mRNA processing. In protein form, a base editor protein may be inefficiently trafficked to the nucleus or may be degraded too quickly. Given that APOBEC1, the cytidine deaminase component of BE4, expresses poorly in bacteria²⁶ it was speculated that altering the coding sequence or codon usage of this component might augment base editing efficiency.

These possibilities were dissected using three fluorescent protein expression experiments. To establish a baseline editing efficiency level among viable cells capable of taking up plasmid DNA during transfection, a three-plasmid mixture consisting of a plasmid expressing BE4, a plasmid expressing an sgRNA, and a separate plasmid expressing mCherry, were co-transfected into human HEK293T cells to mark viable cells that received plasmid. Base editing was measured by high-throughput DNA sequencing (HTS) from mCherry-positive cells isolated by FACS, revealing an average of 45±7.1% C·G-to-T·A editing across the base editing activity window (positions 4-8, counting the PAM as positions 21-23) at five test genomic loci (FIG. 1C, 1D). To examine editing efficiency only among cells that contain the DNA plasmid encoding the base editor, HEK293T cells were transfected with a two-plasmid mixture consisting of a plasmid encoding an sgRNA and a separate plasmid encoding both BE4 and GFP, expressed from separate promoters. Isolation by FACS of GFP-positive cells followed by HTS resulted in an average of 35+7.3% editing at the same five test sites (FIG. 1D). The lack of improvement in editing efficiencies among GFP-positive cells containing the base editor plasmid in the second experiment versus transfected (mCherry-positive) cells in the first experiment suggests that transfection of HEK293T cells was not limiting base editing outcomes.

In a third experiment, editing efficiencies in HEK293T cells were evaluated following transfection of a BE4-P2A-GFP construct, where P2A is a self-cleaving peptide²⁷ that enables co-expression of the GFP protein from the same mRNA transcript as BE4. GFP-positive cells in this experiment can arise only if they also produce full-length BE4 protein. Among GFP-positive cells, base editing efficiencies averaged 65±6.4%, 1.9-fold higher than the average editing efficiencies following transfection of the single plasmid expressing BE4 and GFP from separate promoters (FIG. 1E). These results strongly suggest that the fraction of cells expressing active base editors, and/or the amount of functional base editor protein produced by each cell, are major bottlenecks of base editing efficiency.

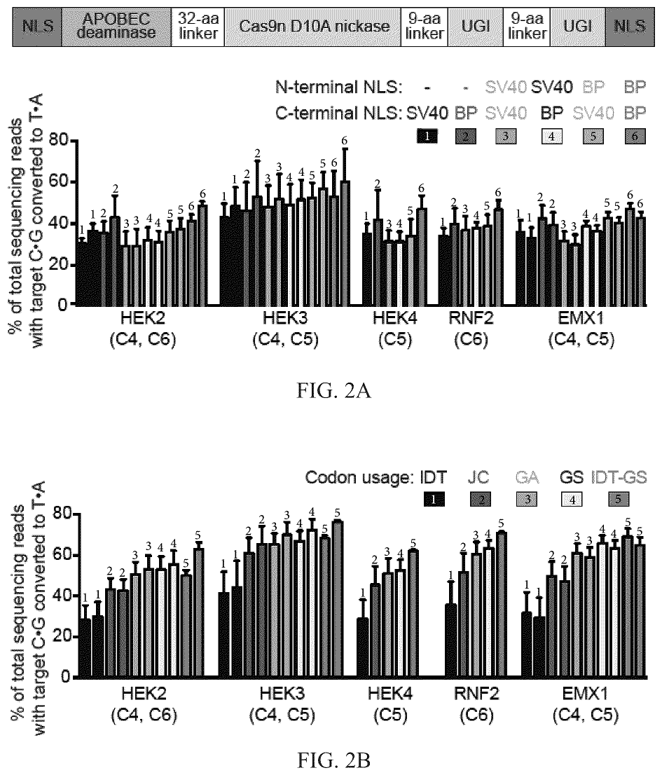

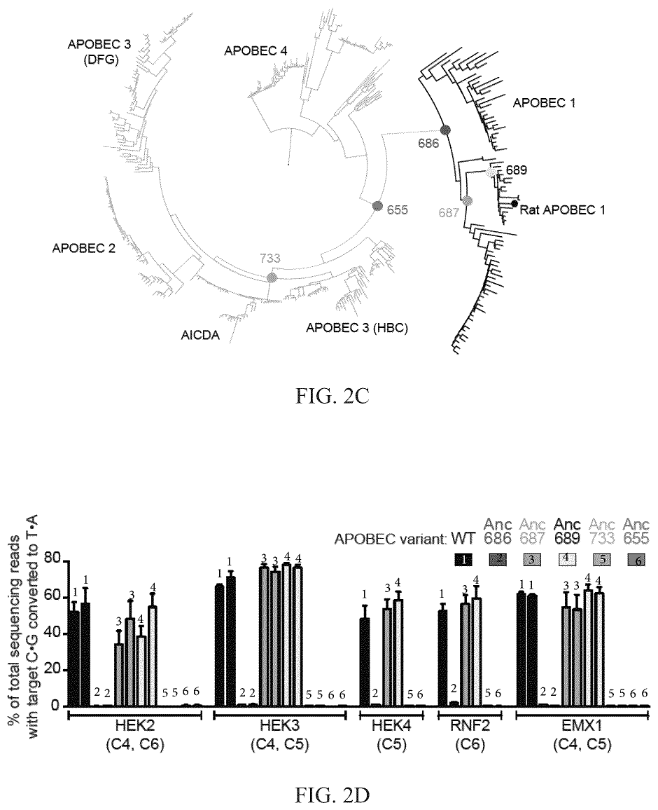

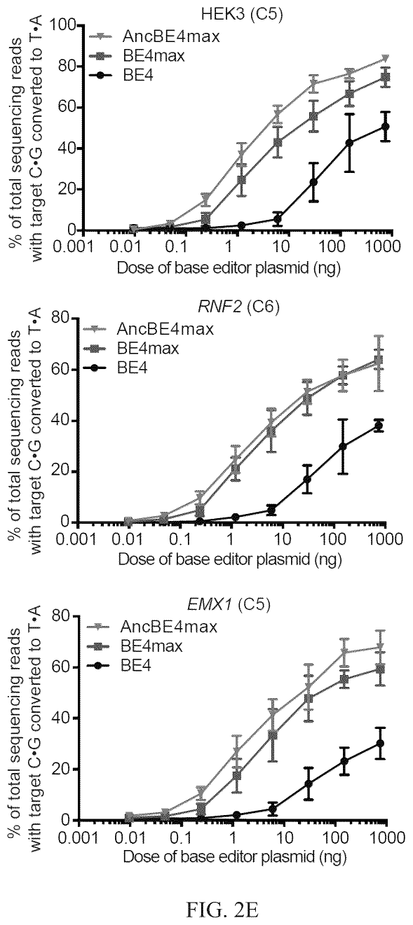

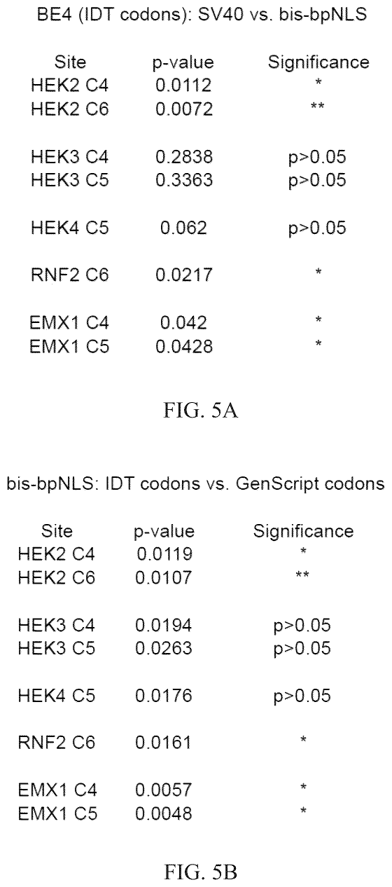

To probe the possibility of sub-optimal nuclear localization, all six combinations of BE4 N- and C-terminal fusion were tested either to the SV40 NLS used in BE4, or to a bipartite NLS (bpNLS) previously shown to improve nuclear localization of Cas9²⁸ (FIG. 2A). All variants using one or two bpNLSs showed improvements in editing efficiency. The presence of a bpNLS at both the N- and C-terminus (referred to hereafter as “bis-bpNLS”) performed best, resulting in a 1.3-fold average improvement in BE4-mediated C·G-to-T·A editing efficiency at five tested genomic loci (48±8.0% average editing compared to 37±5.6% for the C-terminal SV40 NLS used in BE4) (FIG. 2A; see FIG. 5A for p-values). These results together suggest that the use of a bis-bpNLS can significantly improve the editing efficiency of previously described BE3 and BE4^{6, 7}.

Next, it was speculated that improving codon usage might enhance base editor expression. To test this possibility, the improved bis-bpNLS form of BE4 (bpNLS-BE4 bpNLS) coded with eight distinct full-length and chimeric codon optimization strategies was compared. Previously reported BE4 and ABE used codon optimization from IDT (Integrated DNA Technologies)^{3,6}. First C·G-to-T·A editing efficiencies of bis-bpNLS BE4 constructs that use codon optimization from IDT, GeneArt, Coller and co-workers²⁹, and GenScript were compared. At the five endogenous loci in HEK293T cells, all tested new codon optimizations resulted in improved editing efficiency compared to that of the original IDT codons. The best-performing variant used GenScript codons and resulted in an additional 1.8-fold higher editing over bis-bpNLS BE4 with IDT codons, enabling average editing in HEK293T cells of 62+7.8% (FIG. 2B; see FIG. 5B for p-values).

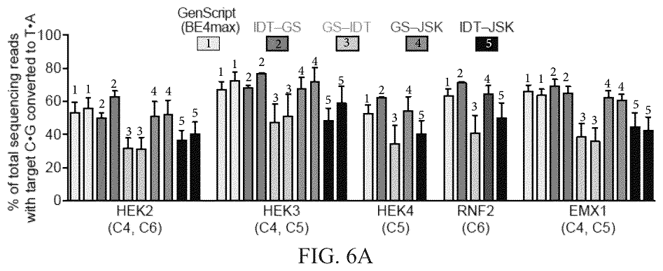

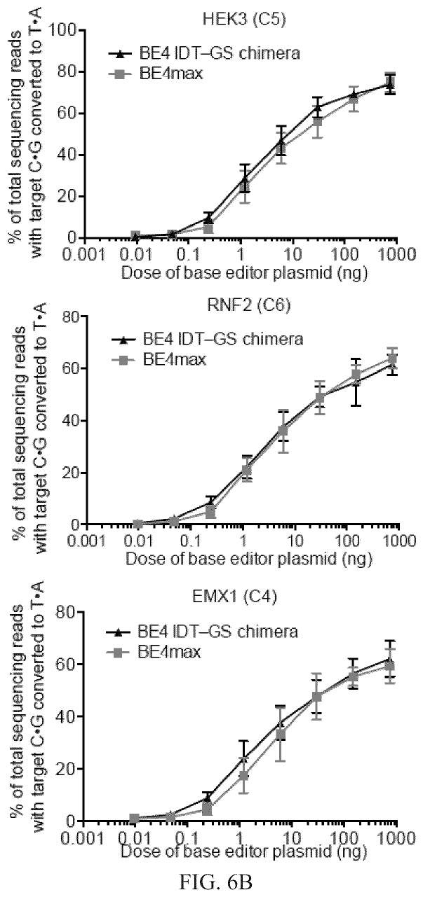

To more deeply dissect the effects of codon optimization, chimeric codon-optimized BE4 variants containing Cas9 nickase coding sequences previously reported by Kim and coworkers to improve expression in human cells,³⁰ were also tested together with cytidine deaminase and UGI domains with codons from IDT or GenScript. The results reveal that both Cas9 nickase and APOBEC codon usage influence BE4 editing efficiency (FIG. 6A). None of the chimeric codon-optimized constructs tested (APOBEC/Cas9 codon usage=IDT/GenScript, GenScript/IDT, GenScript/Kim, or IDT/Kim) resulted in significantly increased BE4 editing efficiencies compared to the optimized bis-bpNLS BE4 editor using full-length GenScript codon optimization, referred to hereafter as BE4max (FIGS. 6A-6B). While the chimera of an IDT deaminase with a GenScript Cas9 nickase showed slightly improved average editing outcomes relative to BE4max at a single high dose of plasmid (FIG. 2B), when tested across an eight-dose plasmid titration at three genomic loci this chimera did not show improved editing compared to BE4max (FIG. 6B). Collectively, these findings establish that codon optimization can dramatically improve the activity of BE4-mediated base editing in human cells.

The above results implicate both APOBEC1 cytidine deaminase and Cas9 nickase expression as key determinants of base editing efficiency (FIG. 2B and FIG. 6A). To further explore strategies for enhancing APOBEC1 expression, ancestral sequence reconstruction (ASR) was performed by maximum likelihood using a set of 468 APOBEC homologs. ASR uses an alignment of known protein sequences, an evolutionary model, and a resulting phylogenetic tree to infer ancestral protein sequences at the nodes of the phylogeny³¹. ASR has been previously shown to greatly improve the expression of a variety of proteins while retaining wild-type levels of biochemical activity³²⁻³⁵. In particular, maximum likelihood reconstructions have been reported to generate sequences with higher expression levels compared to Bayesian reconstructions^{36, 37}. While the reasons for improved expression of ancestral proteins remain actively debated^{32, 36-38}, surprisingly few cases have exploited this phenomenon for protein engineering^{39-41.}

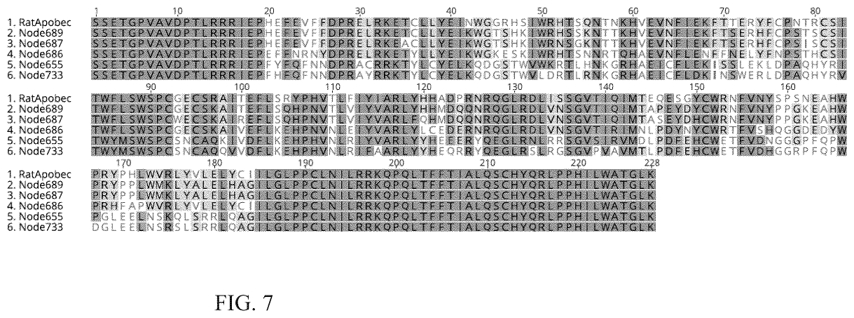

Using the set of 468 APOBEC homologs (“Sequences 5”, below) a maximum likelihood phylogeny was created and the most likely sequences at internal nodes were inferred (FIG. 2C). Five ancestral cytidine deaminases selected at increasing evolutionary distance from rat APOBEC1 used in BE4 were characterized as base editors (FIG. 7). Two ancestors (Anc689 and Anc687) closest in evolutionary distance to the rat APOBEC1 used in BE4 (36 and 45 amino acid differences vs. BE4, respectively) resulted in high editing efficiencies across the five genomic test loci in HEK293T cells, similar to those of BE4max (Anc689: 62±4.9%; Anc687: 57±6.7%; BE4max: 59±4.9%) (FIG. 2D).

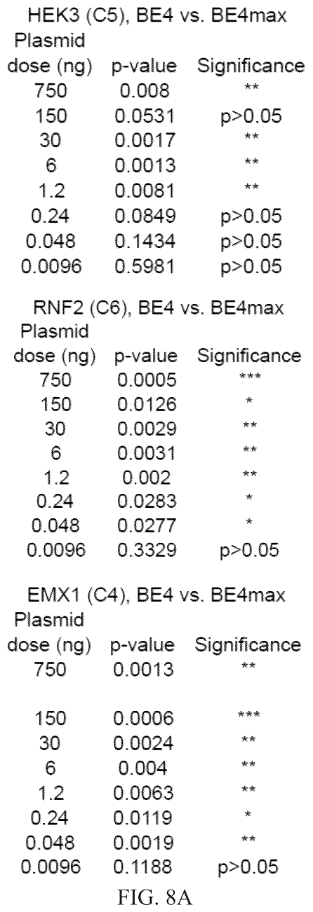

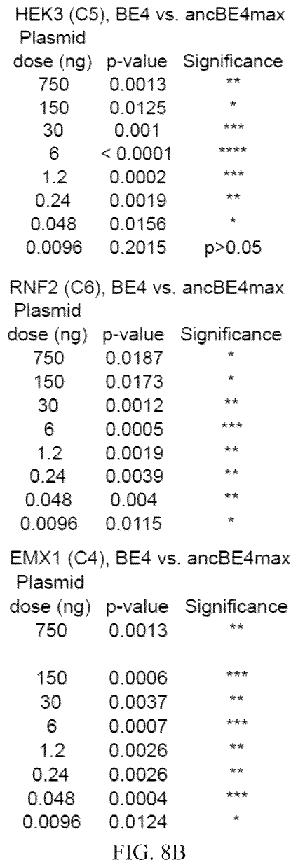

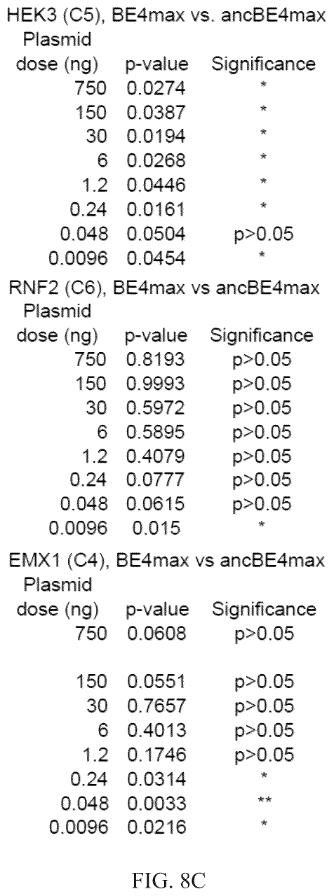

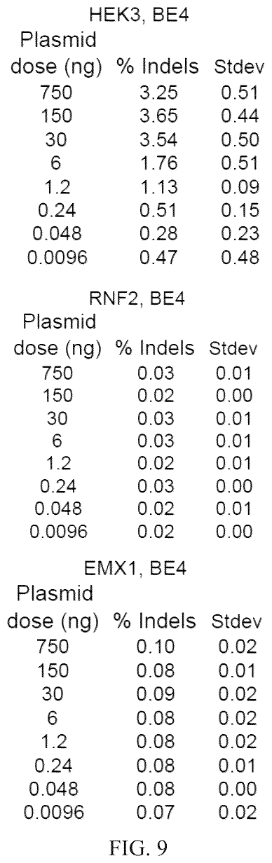

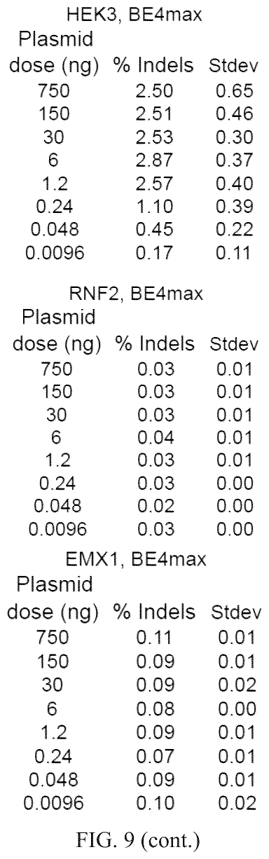

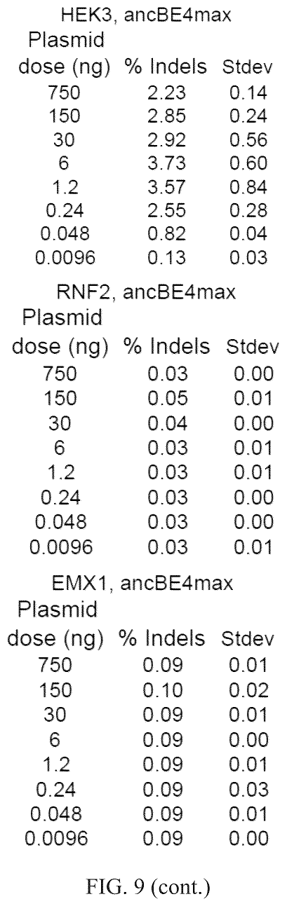

To characterize in depth the base editing activities that result from these improvements, including under sub-optimal conditions, across eight different doses of base editor plasmid at three genomic loci, the editing efficiencies of the previously reported BE46 were compared with those of the three most promising BE4 variants generated in this work: BE4max, bis-npNLS BE4 using the chimeric IDT/GenScript codon optimization, and bis-npNLS BE4 with the Anc689 ancestral APOBEC domain and GenScript codons (referred to hereafter as AncBE4max) (FIG. 2E, p-values reported in FIGS. 8A-8C). The improvements offered by all three of these optimized editors over BE4 are dramatic, ranging from 1.7-fold at higher plasmid doses to >9-fold at lower plasmid doses (FIG. 2E). Ratios of desired point mutation to indels were also improved among all three of these optimized editors compared with the original BE4, as indel frequencies remained similar or lower while base editing improved substantially (FIG. 9). AncBE4max consistently offered the highest activity at target Cs across all three tested sites over a wide range of plasmid doses spanning four orders of magnitude (FIG. 2E; see FIG. 8C for p-values). BE4max resulted in editing efficiencies slightly below, or similar to, those of AncBE4max (FIG. 2E). These data suggest that AncBE4max and BE4max offer large improvements in editing efficiency over BE4, especially under sub-optimal conditions in which factors such as delivery limit overall editing efficiency. As many genome editing applications operate under sub-optimal conditions constrained by poor delivery, or by limited editing opportunities in time or space, it was anticipated that AncBE4max and BE4max will be useful in many settings.

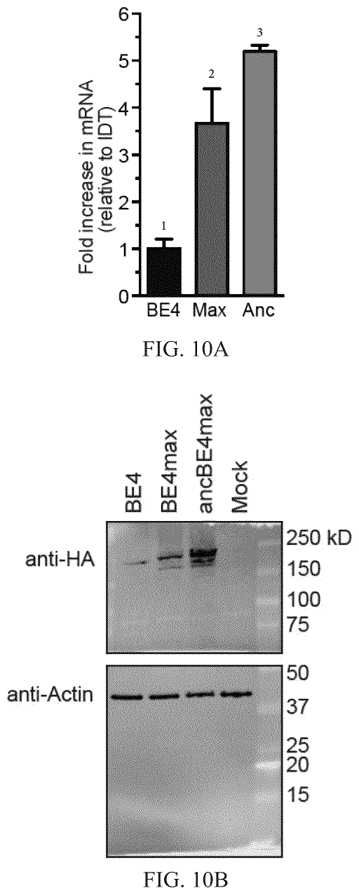

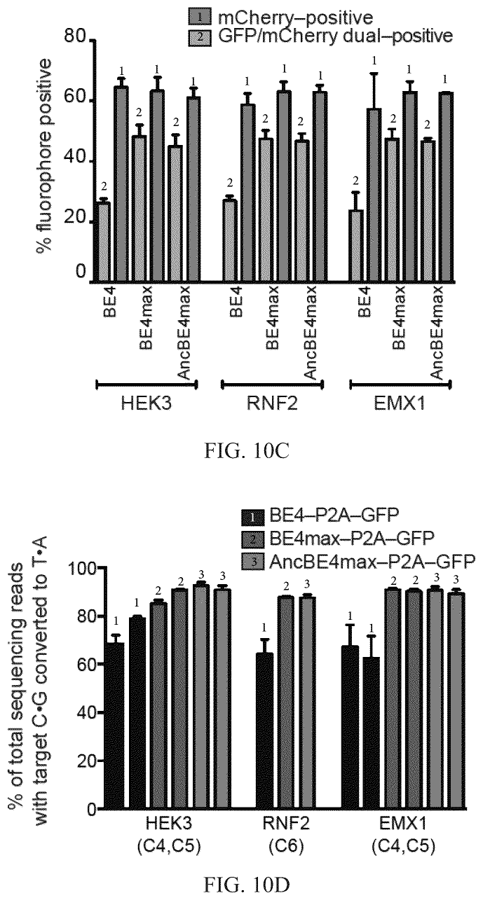

Next, it was determined if the AncBE4max and BE4max result in increased mRNA and protein levels in human cells relative to the previously reported BE4. HEK293T cells were separately transfected with 250 ng of guide RNA and 750 ng of plasmid encoding BE4-P2A-GFP, BE4max-P2A-GFP, or AncBE4max-P2A-GFP. After 3 days, cells were subjected to reverse transcription using GFP-specific primers to evaluate the amount of base editor mRNA present in each sample. BE4max showed >3-fold higher mRNA expression compared to that of BE4, while AncBE4max showed >5-fold higher mRNA expression when compared to BE4 (FIG. 10A). To compare protein levels of BE4 versus BE4max and AncBE4max, HEK293T cells were transfected as described above with plasmids encoding C-terminally epitope-tagged BE4, BE4max, and AncBE4max and performed western blots on the resulting cell lysates. The data reveal that both BE4max and, especially, AncBE4max result in substantial increases in full-length protein abundance relative to actin controls (FIG. 10B). Together, these results indicate that the optimizations that led to BE4max and AncBE4max resulted in major increases in mRNA and protein levels of full-length base editors.

The relationship between improved base editor expression and improved editing efficiency was further illuminated by flow cytometry. HEK293T cells expressing BE4max-P2A-GFP and AncBE4max-P2A-GFP targeted to three test genomic loci were sorted. While the fraction of viable and transfectable (mCherry-positive) cells was very similar among BE4max-P2A-GFP (63±3.8%), AncBE4max-P2A-GFP (62±2.3%), and BE4-P2A-GFP (60±7.0%) across the three genomic sites tested, the frequency of GFP and mCherry double-positive cells, reflecting full-length base editor expression, was on average 1.7-fold higher for both BE4max-P2A-GFP and AncBE4max-P2A-GFP compared to BE4 (FIG. 10C). These improvements in editor expression were reflected in improvements in editing activity. Among mCherry and GFP double-positive cells, BE4max-P2A-GFP showed an average of 89±0.9% target C·G-to-T·A editing across the editing window and AncBE4max-P2A-GFP led to an average of 90±1.5% editing, while double-positive cells expressing BE4-P2A-GFP averaged 48±8.0% (FIG. 1E, FIG. 9). These large improvements demonstrate that isolating cell populations expressing BE4max and AncBE4max results in dramatically higher frequencies of edited cells, which may be especially useful for base editing applications that seek to create novel cell lines, agriculture strains, or animal models.

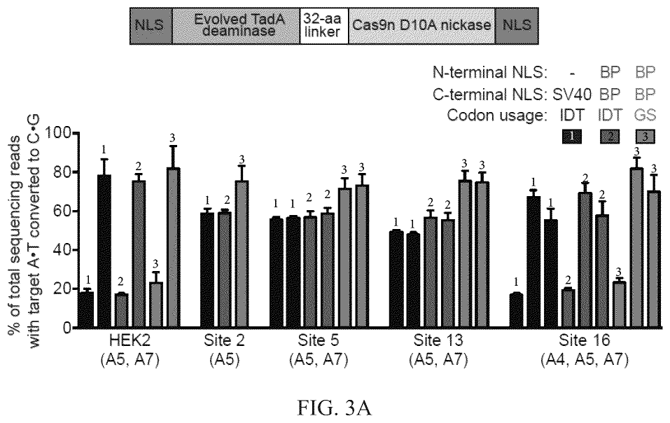

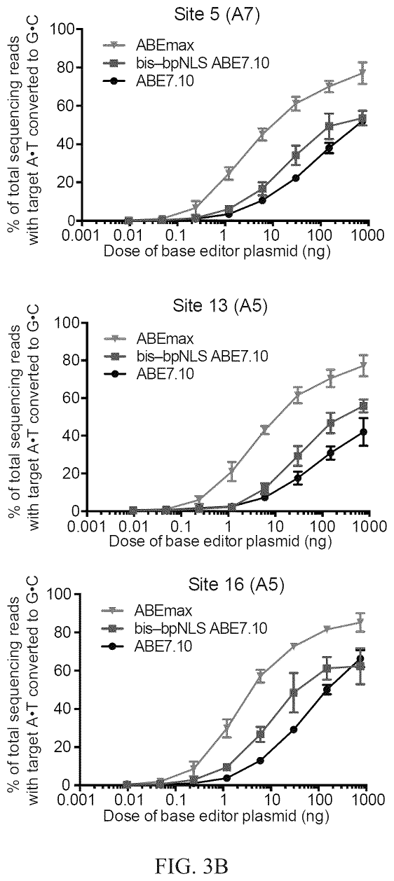

Adenine base editors (ABEs) use a laboratory-evolved deoxyadenosine deaminase and a Cas9 nickase to mediate the conversion of target A·T to G·C base pairs³. Because this conversion reverses the most common class of point mutations in living systems (C·G to T·A)⁴², ABE have the potential to correct a far larger fraction (47%) of pathogenic SNPs than cytidine base editors (14%) (FIG. 1B). Encouraged by the above improvements in BE4 editing efficiency, next, these principles were applied to improve ABE. The applications began with ABE 7.10, the best-performing ABE variant for general use described to date³. While the use of the bis-bpNLS in ABE instead of the previously reported SV40 NLS resulted in little apparent improvement in average A·T-to-G·C editing efficiency in single-dose experiments in HEK293T cells across five human genomic loci test sites (53±3.8% versus 50±3.8% respectively) (FIG. 3A), dose-titration experiments revealed that the bis-bpNLS offered substantially higher ABE editing efficiencies (typically ˜1.5- to 2-fold) at sub-optimal ABE doses (FIG. 3B). These observations indicate that replacement of the SV40 NLS with bis-bpNLS also can enhance ABE-mediated editing efficiency.

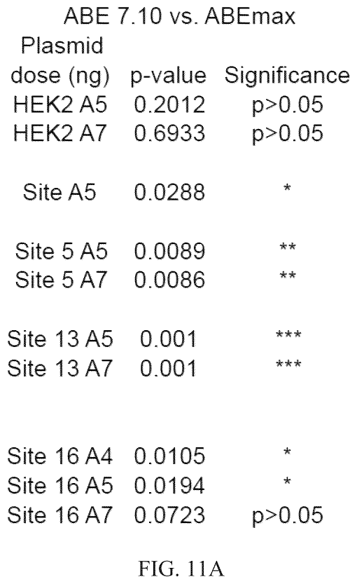

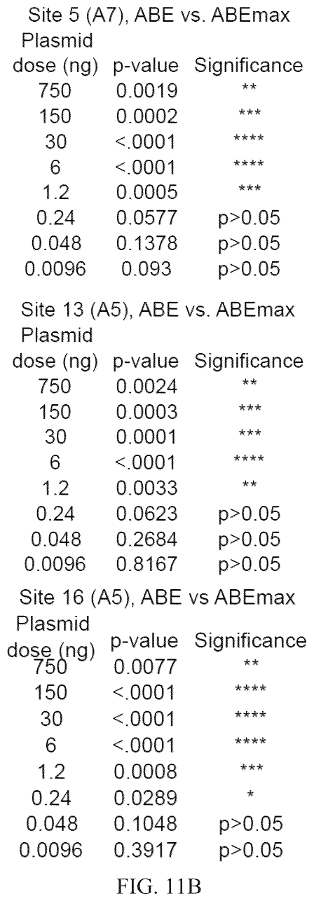

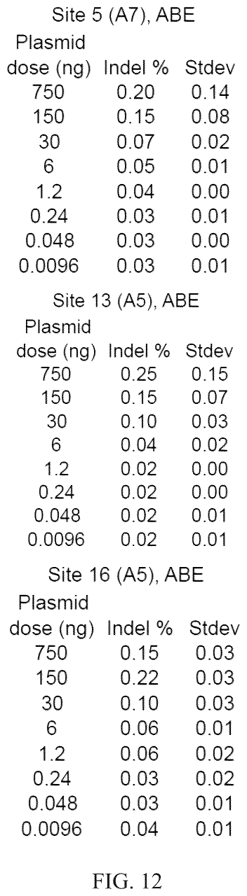

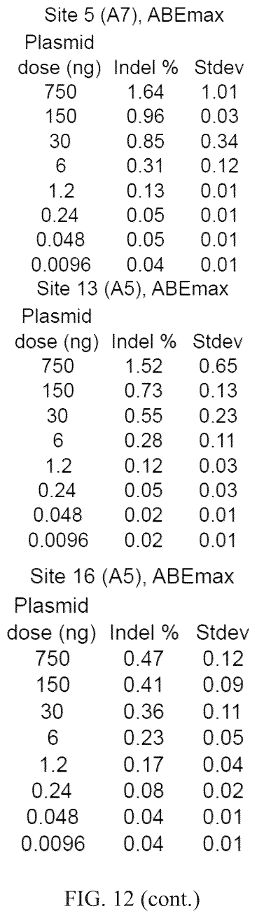

To test the effect of codon optimization on A·T-to-G·C editing efficiency, the original (IDT codons) form of bis-bpNLS-ABE was compared with the Genscript codon optimization form of the same protein at the five test sites in HEK293T cells. Consistent with the findings for BE4, Genscript codon optimization of bis-bpNLS ABE 7.10 (referred to hereafter as ABEmax) also resulted in substantial benefits to editing efficiency compared with IDT codon optimization. At high single-doses of base editor construct, ABEmax resulted in 1.3-fold higher editing levels than ABE (from 50±3.8% to 65±6.8%) across all five sites tested (FIG. 3A; see FIG. 11A for p-values). At sub-optimal doses, ABEmax improved editing efficiencies up to 7.9-fold over those of ABE 7.10 (from 3.2±1.0% to 25±4.4% average editing across three sites tested at low doses) (FIG. 3B; see FIG. 11B for p-values). Although indels from ABEmax remained rare occurrences (≤1.6%), they were elevated from the virtually undetectable indel levels of ABE 7.103 (FIG. 12). Together, these findings establish that improvements in nuclear localization and expression that benefit cytidine base editors are also applicable to ABEs.

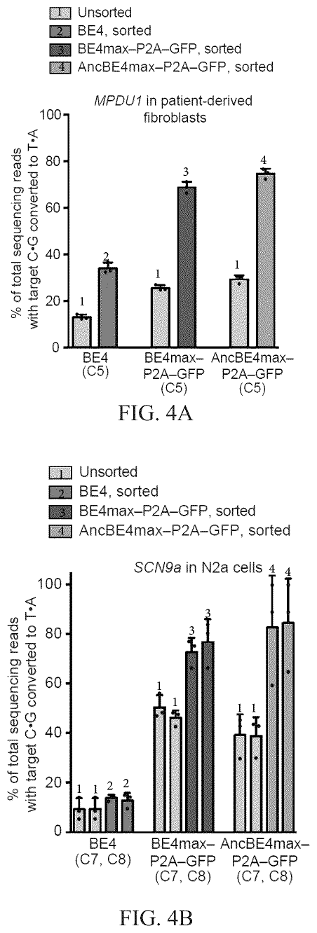

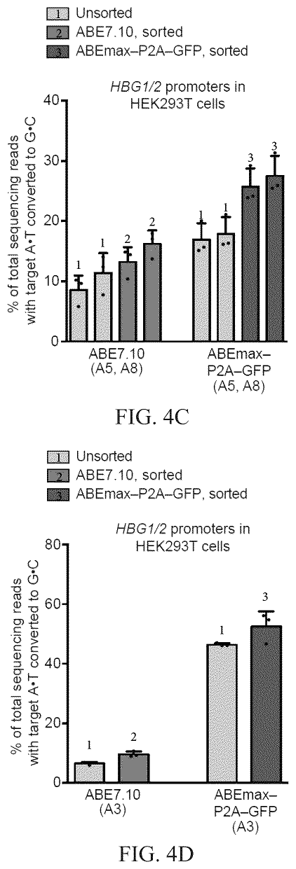

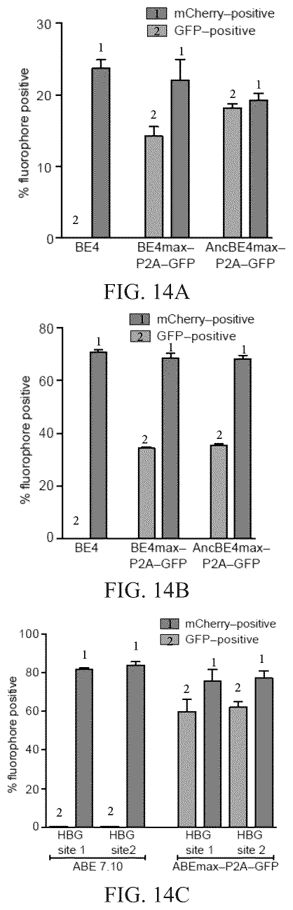

With optimized BE4max, AncBE4max, and ABEmax in hand, it was sought to compare them to previously reported BE4 and ABE7.10 for their ability to edit disease-relevant target loci in a variety of cell types. Patient-derived fibroblasts are commonly used for studying genetic diseases. Mutations in MDPUI, a gene central to N-glycan biosynthesis in the endoplasmic reticulum, gives rise to a condition known as congenital disorder of glycosylation type 1f43. Patient-derived fibroblasts harboring the Leu 119Pro T>C mutation that drives this disease were nucleofected with plasmids expressing BE4, BE4max-P2A-GFP, or AncBE4max-P2A-GFP and the targeting sgRNA. A portion of BE4-treated cells sorted by FACS for an mCherry co-transfection marker, while a portion of cells treated with BE4max-P2A-GFP and AncBE4max-P2A-GFP were sorted for GFP-positive cells. Unsorted cells for BE4 showed 13±1.2% correction of the disease-driving SNP in patient-derived fibroblast, while FACS-sorted BE4 samples showed 34±2.4% correction (FIG. 4A). Unsorted BE4max and AncBE4max resulted in 26±1.3% and 29±1.7% correction of the Leu119Pro mutation, respectively, while sorted cells treated with BE4max and AncBE4max showed 69±2.5% and 75±2.2% SNP correction, respectively (FIG. 4A). Nucleofection efficiencies for all tested conditions were measured by flow cytometry and were consistent across samples, indicating that outcomes were primarily dependent on base editor expression and activity (FIG. 14A). Thus BE4max and AncBE4max resulted in 2.0- and 2.2-fold higher editing efficiencies, respectively, of a pathogenic human SNP in patient-derived fibroblasts.

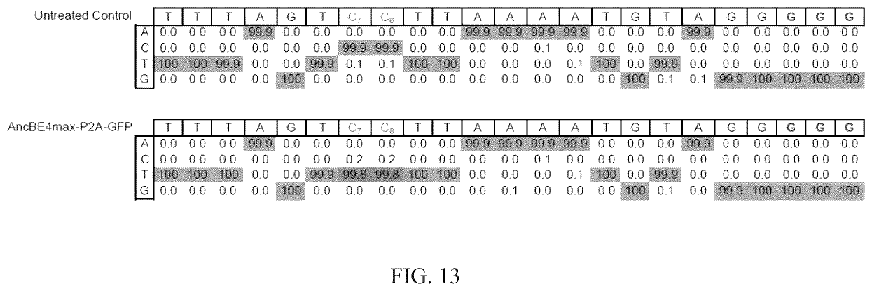

To further test the improvements offered by BE4max and AncBE4max, a splice-modifying mutation was installed in mouse N2a neuroblastoma cells in the voltage-gated sodium channel NaV1.7 (SCN9a gene), a target associated with familial erythromyalgia, paroxysomal extreme pain disorder, and chronic insensitivity to pain^{44, 45}. +1 G and −1 G was targeted in the splice acceptor of SCN9a intron 6 by nucleofection of plasmids encoding BE4, BE4max, or AncBE4max and the targeting sgRNA. BE4 treatment resulted in 9.3+4.4% editing of both +1 G and −1 G among unsorted cells, and 14±1.3% and 13±3.0% editing among sorted cells, respectively (FIG. 4B). BE4max resulted in 50±5.0% and 46±3.1% editing of the target +1 G and −1 G in unsorted cells, and 73±5.7% and 77±9.3% editing among sorted cells. Finally, AncBE4max resulted in 44%±1.7% and 43±0.47% editing of the target +1 G and −1 G in unsorted cells, and 94±7.7% for both target Gs among sorted cells (FIG. 4B). Together, these results demonstrate that BE4max and AncBE4max offer large improvements—here, 5- to 7-fold increases—in editing efficiency of a disease-relevant target in mammalian cells. Indeed, AncBE4max in one sorted sample resulted in 99.8% editing of both target G·C base pairs in SCN9a, demonstrating the very high likelihood of target base editing among cells containing AncBE4max protein (FIG. 13). Transfection efficiencies for all tested conditions were measured by flow cytometry and were consistent across samples, indicating that outcomes were primarily dependent primarily on base editor expression and activity (FIG. 14B).

Finally, the activity of ABE 7.10 versus ABEmax was compared at two blood disease-associated targets. A number of genetic blood disorders, including sickle cell anemia and β-thalassemia, are driven by mutations in the β-globin gene. Activating mutations in the promoters of HBG1 or HBG2 (γ-globin) that are normally silenced after birth can rescue β-globin disorders⁴⁶. Two sgRNAs were designed to install different activating mutations in γ-globin promoters. The first sgRNA should target ABE to introduce mutations at protospacer positions A5 and A8 (HBG promoter positions −116 and −113 relative to the transcription start site). The −116 A to G mutation may perturb the binding site for the γ-globin repressor Bcl11a⁴⁷, while the −113 A to G mutation should perturb this binding site and is also a naturally-occurring SNP that confers the hereditary persistence of fetal hemoglobin⁴⁸. Once again, ABEmax substantially outperformed ABE 7.10, with both unsorted and sorted HEK293T cells treated with ABEmax resulting in approximately double the conversion efficiencies at A5 and A8 than unsorted and sorted cells treated with ABE 7.10 (FIG. 4C). For the second sgRNA, which installs the −175 T to C point mutation in the HBG promoter that is one of the strongest known HBG promoter SNPs upregulating fetal hemoglobin⁴⁸, 6.5±0.57% and 10±1.0% editing in unsorted and sorted HEK293T cells expressing ABE 7.10, was observed respectively, compared to 46±0.55% and 52±5.2% editing in unsorted and sorted cells expressing ABEmax, respectively (FIG. 4D). Thus ABEmax increased editing efficiency at the HBG-175T target by 5.2- to 7.1-fold over ABE 7.10. Notably, this mutation lies at position A3 of the protospacer, and thus is slightly outside the normal ABE editing window, demonstrating that ABEmax can support efficient editing of sites expected to be poor candidates for base editing by ABE 7.10. Transfection efficiencies for all tested conditions were measured by flow cytometry and were consistent across samples, indicating that outcomes were primarily dependent primarily on base editor expression and activity (FIG. 14C). Together, these results demonstrate that ABEmax substantially improves the efficiency of base editing at two loci relevant to human disease.

In summary, elucidation of factors that limit cytidine and adenine base editor efficiency resulted in optimization of nuclear localization and codon usage, as well as ancestral protein reconstruction, greatly improved the expression levels and editing efficiencies of both the BE4 C·G-to-T·A base editor and the ABE 7.10 A·T-to-G·C base editor. The editing performance improvements offered by BE4max, AncBE4max, and ABEmax extended to a variety of mammalian cell types across a number of previously unreported disease-relevant loci, as well as commonly tested loci. These improvements are especially pronounced when using base editors under sub-optimal conditions, such as those commonly found in some research and many therapeutic applications, or at sites that were previously edited with only modest efficiency. These developments greatly expand the capabilities of the current base editors for a wide range of applications, and BE4max, AncBE4max, and ABEmax are recommended for current base editing experiments.

### Methods

General Methods. PCR was performed using either Phusion U Green Multiplex PCR Master Mix (ThermoFisher Scientific) or Q5 Host Start High-Fidelity 2× Master Mix (New England Biolabs) unless otherwise noted. All plasmids were assembled by either the USER cloning method as previously described⁴⁹ or by Gibson assembly⁵⁰. Plasmids for mammalian cell transfections were prepared using an endotoxin removal plasmid purification system, ZymoPURE Plasmid Midiprep (Zymo Research Corporation).

Cell culture conditions. HEK293T cells (ATCC CRL-3216) were cultured in Dulbecco's Modified Eagle's Medium (DMEM, Corning) supplemented with 10% fetal bovine serum (FBS) and 5% penicillin streptomycin (Pen Strep, ThermoFisher Scientific). Fibroblast cell lines were maintained in DMEM supplemented with 15% FBS. N2a cells were maintained in DMEM supplemented with 10% FBS.

HEK293T transfection and genomic DNA preparation. HEK293T cells were seeded into 48-well Poly-D-Lysine coated plates (Corning) in the absence of Pen Strep antibiotic. 12-15 hours after plating, cells were transfected with 1 μL of Lipofectamine 2000 (ThermoFisher Scientific) using 750 ng of base editor plasmid, 250 ng of guide RNA plasmid, and 20 ng of fluorescent protein expression plasmid as a transfection control. Unless otherwise stated, cells were cultured for 3 days before they were washed with PBS (ThermoFisher Scientific). Genomic DNA was extracted by addition of 150 μL of freshly prepared lysis buffer (10 mM Tris-HCl, pH 7.5, 0.05% SDS, 25 μg/mL proteinase K (ThermoFisher Scientific)) directly into each transfected well. The resulting mixture was incubated for 1 hour at 37° C. before a 30-min enzyme inactivation step at 80° C. Guide RNA sequences for HEK2, HEK3, HEK4, RNF2, EMX1, Site 2, Site 5, Site 13, Site 16 were previously reported^{2, 3, 6}.

HEK293T base editing dose titrations. HEK293T cells were seeded as described above and transfected with a mixture of base editor plasmid, guide RNA plasmid, pUC, and GFP. 250 ng of guide RNA plasmid and 20 ng of GFP transfection control plasmid were used for all samples. Base editor and pUC plasmids were combined in different amounts to maintain a constant amount of total DNA per transfection.

Fluorescence-activated cell sorting. Flow cytometry analysis was carried out using an Aria Fortessa III. HEK293T cells were transfected with guide RNA expression plasmids, fluorophore expression plasmids, and editor expression plasmids. In trans samples were sorted for mCherry-positive cells. Both the in cis and P2A samples were sorted for both GFP and mCherry double-positive cells. A stringent mCherry-positive gate was used to avoid mCherry false positives. Over 15,000 cells were collected for each experimental sample. Genomic DNA for sorted and unsorted FACS samples was isolated using the Agencourt DNAdvance Genomic DNA Isolation Kit (Beckman Coulter) according to the manufacturer's instructions.

Nucleofection of fibroblasts and genomic DNA extraction. Cells were nucleofected using the Primary P2 Cell Line 4D-Nucleofector X Kit (Lonza) according to manufacturer's protocol. 1.25×10⁵ cells were nucleofected in 20 μL of P2 buffer supplemented with 750 ng of editor, 250 ng of guide RNA plasmid, and 20 ng of mCherry nucleofection marker. Cells were nucleofected in a 16-well nucleocuvette strip using the DT-130 program. Following a 3-day incubation, cells were flow sorted and genomic DNA was extracted as described for HEK293T cells above.

High-throughput DNA sequencing (HTS) of genomic DNA. HTS of genomic DNA from HEK293T cells was perform as described previously^{2, 3, 6}. For fibroblasts, 34 cycles of amplification were used for PCR1. Primers for PCR 1 of HEK2, HEK3, HEK4, RNF2, EMX1, ABE Site 2, ABE Site 5, ABE Site 13, ABE Site 16, and HBG loci were used as previously described^{3, 6, 19}. PCR 1 primers for type 1F congenital glycosylation disorder, SCN9a, and all previously used loci are listed in Sequences 2 below.

General HTS analysis. Sequencing reads were demultiplexed using the MiSeq Reporter (Illumina) and Fastq files were analyzed using using open source analysis tools. FASTQ files were aligned to the reference genome using the burrows-wheeler aligner (bwa-mem)⁵¹. Statistics for each base were calculated using the pysamstats utility available at

github.com/alimanfoo/pysamstats. All reads for a given base were aligned to the reference sequence. Total reads were the sum of all base calls, insertions, and deletions at any given nucleotide position. Percent representation of each base was calculated as reads of a given base divided by total reads. Indel frequencies were quantified with a custom Matlab script as previously described^{3, 20}.

Quantitative RT-PCR and quantitative PCR. HEK293T cells were transfected with base editor-P2A-GFP plasmids and incubated 3 days before harvesting DNA and RNA from each sample. DNA samples were harvested using the genomic DNA prepartion protocol described above. RNA was isolated and amplified using the Cells-to-Ct (Thermofisher) kit according to the manufacturer's protocol except the DNase treatment step used 2X DNase for twice as long to ensure complete degradation of plasmid DNA. Levels of mRNA were calculated by normalizing base editor mRNA levels to β-actin levels by ΔΔCt. Plasmid DNA levels were calculated to ensure that mRNA levels were not skewed by transfection efficiency. Plasmid DNA levels were calculated by normalizing amplification of the BGH poly-adenylation present on the base editor plasmid to β-actin levels.

Western blotting. HEK293T cells were transfected with 750 ng of base editor-3X HA tag plasmid and 250 ng of guide RNA plasmid. After 3 days, cells were lysed using RIPA buffer with PMSF and complete Protease Inhibitor Cocktail (Sigma-Aldrich). Samples were boiled and quantified using a BCA assay. 10 μg of protein was loaded per well into a 12-well 4-12% Tris gel (Novex). Blots were transferred to nitrocellulose paper for 7 min at 20 V before blocking and incubation with anti-HA (Cell Signaling Technology) and anti-Actin antibodies (Cell Signaling Technology). Blots were visualized using an Odyssey imager.

APOBEC sequence collection. APOBEC protein sequences used in phylogenetic analyses were identified through searches of the Uniprot database⁵² with the BLASTP algorithm⁵³ using selected query sequences. All sequences from these searches that returned BLASTP E-values <10⁻⁷ were downloaded from Uniprot. To reduce phylogenetic complexity, sequences were curated based on character length and pairwise sequence identity within each dataset. The dataset used for the construction of the non-redundant phylogeny was generated using four query sequences: UniProt IDs P41238, H2P4E7, EIBTD6, and H2P4E9. Multiple sequences were necessary to generate full coverage due to the low sequence identity across the family, which is <25% between some members. Limits were chosen to remove truncated and partial sequences and those featuring large insertions or terminal extensions. Sequences greater than 97% identical, determined by pairwise alignment within the dataset, were also removed. This level of identity provides a high level of detail within the tree while accelerating computational time by removing redundant taxa. The final dataset contains 468 taxa (Sequences 5 below).

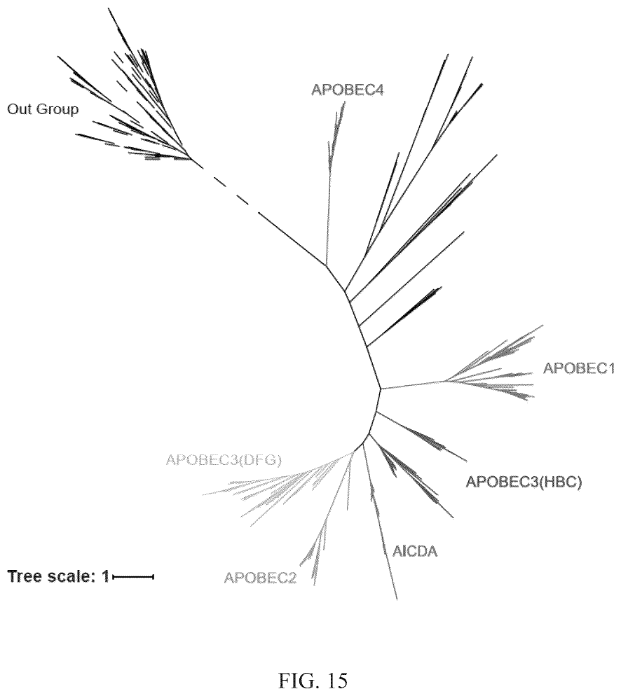

Phylogeny construction. A multiple sequence alignment of the dataset was generated with the program MAFFT using the FFT-NS-I x1000 algorithm⁵⁴. Model selection used the Bayesian information criteria (BIC) to determine the evolutionary model that best fit the input alignment⁵⁵. 228 models where tested. The Jones Taylor Thornton (JTT) substitution matrix with empirical frequencies (F) and free rates with five categories (R5) was the model that best fit the data. A maximum likelihood (ML) phylogenetic tree was inferred with IQ-TREE⁵⁶ using the best fit model (JTT+F+R5). The starting trees were generated by randomized maximum parsimony and searched by fast hill-climbing Nearest Neighbor Interchange (NNI). Tree topology, branch lengths, and rate parameters were optimized. Branch supports were estimated with Ultrafast boot strapping, implemented in IQ-TREES7 (FIG. 15).

Ancestral sequence reconstruction. Sequences at internal nodes in the phylogeny were inferred using the codeml program from the PAML software package58. Posterior amino acid probabilities at each site were calculated using the JTT substitution matrix, given the ML tree and estimated background frequencies generated by IQ-TREE. N- and C-termini of ancestral sequences were modified manually to match those of Rat APOBEC1.

Note 1. Python script to analyze pathogenic SNPs within the Clin Var database.
import numpy as np
import pandas as pd
#download latest Clin Var from //ftp.ncbi.nlm.nih.gov/pub/clinvar/tab_delimited/
#convert to csv
ClinVar=pd.read_csv (′2018 Apr. 23-variant_summary.csv′)
#restrict to SNPs
ClinVar=ClinVar [ClinVar.Type== ‘single nucleotide variant’]
#restrict to pathogenic
ClinVar=Clin Var [ClinVar.ClinicalSignificance== ‘Pathogenic’]
#remove nans
ClinVar=Clin Var [Clin Var.ReferenceAllele!= ‘na’]
ClinVar=Clin Var [Clin Var.AlternateAllele!= ‘na’]
#drop duplicates of AlleleID
ClinVar=ClinVar.drop_duplicates(‘#AlleleID’)
#total SNPs in Clin Var
total_SNPs=len (ClinVar)
def SNP_count (cv, ref, alt):
ClinVar_ref=cv [cv.ReferenceAllele==ref]
ClinVar_ref_alt=Clin Var_ref [ClinVar_ref.AlternateAllele==alt]
return len (Clin Var_ref_alt)
counts=np.array ([[“, ‘A’, ‘T’, ‘G’, ‘C’],
[′A′, 0, SNP_count (ClinVar, ‘A’, ‘T’), SNP_count (ClinVar, ‘A’, ‘G’), SNP_count (ClinVar, ‘A’, ‘C’)],
[′T′, SNP_count (ClinVar, ‘T’, ‘A’), 0, SNP_count (ClinVar, ‘T’, ‘G’), SNP_count (Clin Var, ‘T’, ‘C’)],
[′G′, SNP_count (ClinVar, ‘G’, ‘A’), SNP_count (ClinVar, ‘G’, ‘T’), 0, SNP_count (ClinVar, ‘G’, ‘C’)],
[′C′, SNP_count (ClinVar, ‘C’, ‘A’), SNP_count (ClinVar, ‘C’, ‘T’), SNP_count (ClinVar, ‘C’, ‘G’), 0]])
np.savetxt (′ClinVar_SNPs.csv′, counts, fmt=′% 5s′, delimiter=′,′)
   Sequences 1. Target Protospacer Sequences Used in this Study.

Target Cs and As are bold, with a subscripted number denoting spacer position. PAM sequences are italicized.

| HEK293_site | GAAC4AC6AAAGCATAGACTG | SEQ ID NO: |
|---|---|---|
| | CGGG | 150 |
| HEK293_site | GGCC4C5AGACTGAGCACGTG | SEQ ID NO: |
| 3 | ATGG | 151 |
| HEK293_site | GGCAC5TGCGGCTGGAGGTCC | SEQ ID NO: |
| | GGG | 152 |
| RNF2 | GTCATC6TTAGTCATTACCTG | SEQ ID NO: |
| | AGG | 153 |
| EMX1 | GAGTC5C6GAGCAGAAGAAGA | SEQ ID NO: |
| | AGGG | 154 |
| SCN9a | GTTAGTC7C8TTAAAATGTAG | SEQ ID NO: |
| | GGGGG | 155 |
| MPDU1 | GTTC4C5C6GGTC10ATGCAC | SEQ ID NO: |
| | TACAGAGG | 156 |
| ABE_site 2 | GAGTA5TGA7GGCATAGACTG | SEQ ID NO: |
| | CAGG | 157 |
| ABE_site 5 | GATGA5GA7TAATGATGAGTC | SEQ ID NO: |
| | AGGG | 158 |
| ABE_site 13 | GAAGA5TA7GAGAATAGACTG | SEQ ID NO: |
| | CTGG | 159 |
| ABE_site 16 | GGGA4A5TA7AATCATAGAAT | SEQ ID NO: |
| | CCTGG | 160 |
| HBG_site 1 | CTTGA5CCA8A9TA11GCCTT | SEQ ID NO: |
| | GACAAGG | 161 |
| HBG_site 2 | A1TA3TTTGCA9TTGA13GAT | SEQ ID NO: |
| | AGTGTGG | 162 |

Sequences 2. Primers used in this study.

All oligonucleotides were purchased from Integrated DNA Technologies (IDT).

Primers Used for Generating sgRNA Plasmids

The MPDU1 guide plasmid was cloned by digesting a modified version of pFYF13201 in which BsmBI restriction cut sites were installed via KLD cloning. The primers below were phosphorylated and annealed to enable ligation into BsmBI cut backbone. All guides were designed to include a 5′-G to enable transcription from the hU6 transcription. CCACC was included at the 5′ end of the forward primer, and AAAC was included at the 5′ end of the reverse primer to complement the overhands generated by restriction digest. The HEK2, HEK3, HEK4, RNF2, EMX1, ABE site 2, ABE site 5, ABE site 13, ABE site 16, SCN9a, HBG site 1, HBG site 2 sgRNAs were prepared by KLD cloning as previously described² using the primers listed below.

Primers for MPDUI sgRNA

JLD 85
(SEQ ID NO: 163)
CACCGTTCCCGGTCATGCACTACAG
JLD 86
(SEQ ID NO: 164)
AAACCTGTAGTGCATGACCGGGAAC

Primers for SCN9a, HBG Site 1, HBG Site 2, and Previously Used sgRNAs

Universal reverse primer
(SEQ ID NO: 165)
GGTGTTTCGTCCTTTCCACAAG
fwd_HEK293_site 2
(SEQ ID NO: 166)
GAACACAAAGCATAGACTGCGTTTTAGAGCTAGAAATAGCAAGTTAAAAT
AAGGC
fwd_HEK293_site 3
(SEQ ID NO: 167)
GGCCCAGACTGAGCACGTGAGTTTTAGAGCTAGAAATAGCAAGTTAAAAT
AAGGC
fwd_HEK293_site 4
(SEQ ID NO: 168)
GGCACTGCGGCTGGAGGTGGGTTTTAGAGCTAGAAATAGCAAGTTAAAAT
AAGGC
fwd_RNF2
(SEQ ID NO: 169)
GTCATCTTAGTCATTACCTGGTTTTAGAGCTAGAAATAGCAAGTTAAAAT
AAGGC
fwd_EMX1
(SEQ ID NO: 170)
GAGTCCGAGCAGAAGAAGAAGTTTTAGAGCTAGAAATAGCAAGTTAAAAT
AAGGC
fwd_ABE_site 2
(SEQ ID NO: 171)
GAGTATGAGGCATAGACTGCGTTTTAGAGCTAGAAATAGCAAGTTAAAAT
AAGGC
fwd_ABE_site 5
(SEQ ID NO: 172)
GATGAGATAATGATGAGTCAGTTTTAGAGCTAGAAATAGCAAGTTAAAAT
AAGGC
fwd_ABE_site 13
(SEQ ID NO: 173)
GAAGATAGAGAATAGACTGCGTTTTAGAGCTAGAAATAGCAAGTTAAAAT
AAGGC
fwd_ABE_site 16
(SEQ ID NO: 174)
GGGAATAAATCATAGAATCCGTTTTAGAGCTAGAAATAGCAAGTTAAAAT
AAGGC
fwd_SCN9a
(SEQ ID NO: 175)
GTTAGTCCTTAAAATGTAGGGGTTTTAGAGCTAGAAATAGCAAGTTAAAA
TAAGGC
fwd_HBG_site 1
(SEQ ID NO: 176)
GCTTGACCAATAGCCTTGACAGTTTTAGAGCTAGAAATAGCAAGTTAAAA
TAAGGC
fwd_HBG_site 2
(SEQ ID NO: 177)
GATATTTGCATTGAGATAGTGGTTTTAGAGCTAGAAATAGCAAGTTAAAA
TAAGGC

### Primers to Amplify Genomic Loci for HTS of Mammalian Cell Culture Experiments

SCN9a HTS Fwd
(SEQ ID NO: 178)
ACACTCTTTCCCTACACGACGCTCTTCCGATCTNNNNGCGAACACACTGA
GACAGAAC
SCN9a HTS Rev
(SEQ ID NO: 179)
TGGAGTTCAGACGTGTGCTCTTCCGATCTGCACTCCTAGTTAGGCTTGTG
Type 1f HTS Fwd
(SEQ ID NO: 180)
ACACTCTTTCCCTACACGACGCTCTTCCGATCTNNNNGTCCCTGGATGGA
TGGGCTATGG
Type 1f HTS Rev
(SEQ ID NO: 181)
TGGAGTTCAGACGTGTGCTCTTCCGATCTGGCTTTCCCAGACCTGAGTTC
CC
HBG HTS Fwd
(SEQ ID NO: 182)
ACACTCTTTCCCTACACGACGCTCTTCCGATCTNNNNCCTGGCCTCACTG
GATACTC
HBG HTS Rev
(SEQ ID NO: 183)
TGGAGTTCAGACGTGTGCTCTTCCGATCTCTGACAAAAGAAGTCCTGGTA
TC
fwd_HEK293_site 2_HTS
(SEQ ID NO: 184)
ACACTCTTTCCCTACACGACGCTCTTCCGATCTNNNNCCAGCCCCATCTG
TCAAACT
rev_HEK293_site 2_HTS
(SEQ ID NO: 185)
TGGAGTTCAGACGTGTGCTCTTCCGATCTTGAATGGATTCCTTGGAAACA
ATGA
fwd_HEK293_site 3_HTS
(SEQ ID NO: 186)
ACACTCTTTCCCTACACGACGCTCTTCCGATCTNNNNATGTGGGCTGCCT
AGAAAGG
rev_HEK293_site 3_HTS
(SEQ ID NO: 187)
TGGAGTTCAGACGTGTGCTCTTCCGATCTCCCAGCCAAACTTGTCAACC
fwd_HEK293_site 4_HTS
(SEQ ID NO: 188)
ACACTCTTTCCCTACACGACGCTCTTCCGATCTNNNNGAACCCAGGTAGC
CAGAGAC
rev_HEK293_site 4_HTS
(SEQ ID NO: 189)
TGGAGTTCAGACGTGTGCTCTTCCGATCTTCCTTTCAACCCGAACGGAG
fwd_RNF2_HTS
(SEQ ID NO: 190)
ACACTCTTTCCCTACACGACGCTCTTCCGATCTNNNNACGTCTCATATGC
CCCTTGG
rev_RNF2_HTS
(SEQ ID NO: 191)
TGGAGTTCAGACGTGTGCTCTTCCGATCTACGTAGGAATTTTGGTGGGAC
A
fwd_EMX1_HTS
(SEQ ID NO: 192)
ACACTCTTTCCCTACACGACGCTCTTCCGATCTNNNNCAGCTCAGCCTGA
GTGTTGA
rev_EMX1_HTS
(SEQ ID NO: 193)
TGGAGTTCAGACGTGTGCTCTTCCGATCTCTCGTGGGTTTGTGGTTGC
fwd_ABE_site 2_HTS
(SEQ ID NO: 194)
ACACTCTTTCCCTACACGACGCTCTTCCGATCTNNNNAGAGACTGATTGC
GTGGAGT
rev_ABE_site 2_HTS
(SEQ ID NO: 195)
TGGAGTTCAGACGTGTGCTCTTCCGATCTCACTCCAGCCTAGGCAACAA
fwd_ABE_site 5_HTS
(SEQ ID NO: 196)
ACACTCTTTCCCTACACGACGCTCTTCCGATCTNNNNGTCTGAGGTCACA
CAGTGGG
rev_ABE_site 5_HTS
(SEQ ID NO: 197)
TGGAGTTCAGACGTGTGCTCTTCCGATCTCTGAGAGCAGGGACCACATC
fwd_ABE_site 13_HTS
(SEQ ID NO: 198)
ACACTCTTTCCCTACACGACGCTCTTCCGATCTNNNNTCACTTCAGCCCA
GGAGTAT
rev_ABE_site 13_HTS
(SEQ ID NO: 199)
TGGAGTTCAGACGTGTGCTCTTCCGATCTTCTCTTTCTCTCCCCCACCC
fwd_ABE_site 16_HTS
(SEQ ID NO: 200)
ACACTCTTTCCCTACACGACGCTCTTCCGATCTNNNNGGGAGGTGGAGAG
AGGATGT
rev_ABE_site 16_HTS
(SEQ ID NO: 201)
TGGAGTTCAGACGTGTGCTCTTCCGATCTTCCTGAGGTCTAGGAACCCG

Sequences 3. Amino Acid Sequences of BE4, rAPOBEC1, Ancestral APOBECs, ABE, and P2A-GFP.

Within base editor sequences, NLS sequences are bold, APOBEC and TadA sequences are italicized, linkers are double underlined, Cas9 nickase sequence is underlined, and UGI sequences are bold and italicized.

BE4max and AncBE4max
(SEQ ID NO: 202)
MKRTADGSEFESPKKKRKV_[APOBEC]_SGGSSGGSSGSETPGTSESATPESSGGSS
GGS DKKYSIGLAIGTNSVGWAVITDEYKVPSKKFKVLGNTDRHSIKKNLIGALLEDS
GETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLI
EGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEY
FTVYNELTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTLTLFEDREMIE
ERLKTYAHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPVEN
TQLQNEKLYLYYLQNGRDMYVDQELDINRLSDYDVDHIVPQSFLKDDSIDNKVLTR
SDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRK
VLSMPQVNIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLGGD SGGSG
GSGGS TNLSDIIEKETGKQLVIQESILMLPEEVEEVIGNKPESDILVHTAYDESTDEN
VMLLTSDAPEYKPWALVIQDSNGENKIKML SGGSGGSGGS TNLSDIIEKETGKQLV
IQESILMLPEEVEEVIGNKPESDILVHTAYDESTDENVMLLTSDAPEYKPWALVIQDS
NGENKIKML SGGS KRTADGSEFEPKKKRKV [optional P2A-GFP]

The portion indicated by [APOBEC] in the above sequence of SEQ ID NO 202 may include any APOBEC sequence, or variant thereof, provided herein. For example, in some embodiments, the [APOBEC] may comprise any one of SEQ ID NOs: 4-9, 203-209, or 220-687.

Exemplary BE4max(with nickase):
(SEQ ID NO: 693)
MKRTADGSEFESPKKKRKVSSETGPVAVDPTLRRRIEPHEFEVFFDPRELRKETCLLY
EINWGGRHSIWRHTSQNTNKHVEVNFIEKFTTERYFCPNTRCSITWFLSWSPCGECSR
AITEFLSRYPHVTLFIYIARLYHHADPRNRQGLRDLISSGVTIQIMTEQESGYCWRNFV
NYSPSNEAHWPRYPHLWVRLYVLELYCIILGLPPCLNILRRKQPQLTFFTIALQSCHY
QRLPPHILWATGLKSGGSSGGSSGSETPGTSESATPESSGGSSGGSDKKYSIGLAIGTN
SVGWAVITDEYKVPSKKFKVLGNTDRHSIKKNLIGALLFDSGETAEATRLKRTARRR
YTRRKNRICYLQEIFSNEMAKVDDSFFHRLEESFLVEEDKKHERHPIFGNIVDEVAYH
EKYPTIYHLRKKLVDSTDKADLRLIYLALAHMIKFRGHFLIEGDLNPDNSDVDKLFIQ
LVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLS
DILRVNTEITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFE
EVVDKGASAQSFIERMTNFDKNLPNEKVLPKHSLLYEYFTVYNELTKVKYVTEGMR
KPAFLSGEQKKAIVDLLFKTNRKVTVKQLKEDYFKKIECFDSVEISGVEDRFNASLGT
YHDLLKIIKDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDSLTFKEDIQ
KAQVSGQGDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYLQNGRDM
YVDQELDINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQITKHVAQIL
DSRMNTKYDENDKLIREVKVITLKSKLVSDFRKDFQFYKVREINNYHHAHDAYLNA
VVGTALIKKYPKLESEFVYGDYKVYDVRKMIAKSEQEIGKATAKYFFYSNIMNFFKT
EITLANGEIRKRPLIETNGETGEIVWDKGRDFATVRKVLSMPQVNIVKKTEVQTGGFS
KESILPKRNSDKLIARKKDWDPKKYGGFDSPTVAYSVLVVAKVEKGKSKKLKSVKE
LLGITIMERSSFEKNPIDFLEAKGYKEVKKDLIIKLPKYSLFELENGRKRMLASAGELQ
KGNELALPSKYVNFLYLASHYEKLKGSPEDNEQKQLFVEQHKHYLDEIIEQISEFSKR
VILADANLDKVLSAYNKHRDKPIREQAENIIHLFTLTNLGAPAAFKYFDTTIDRKRYT
STKEVLDATLIHQSITGLYETRIDLSQLGGDSGGSGGSGGSTNLSDIIEKETGKQLVIQE
SILMLPEEVEEVIGNKPESDILVHTAYDESTDENVMLLTSDAPEYKPWALVIQDSNGE
NKIKMLSGGSGGSGGSTNLSDIIEKETGKQLVIQESILMLPEEVEEVIGNKPESDILVHT
AYDESTDENVMLLTSDAPEYKPWALVIQDSNGENKIKMLSGGSKRTADGSEFEPKK
KRKV
Exemplary BE4 sequence:
(SEQ ID NO: 688)
MSSETG PVAVDPTLRR RIEPHEFEVF FDPRELRKET CLLYEINWGGRHSIWRHTSQ
NTNKHVEVNF IEKFTTERYF CPNTRCSITW FLSWSPCGEC
SRAITEFLSRYPHVTLFIYI ARLYHHADPR NRQGLRDLIS SGVTIQIMTE
QESGYCWRNF VNYSPSNEAH WPRYPHLWVR LYVLELYCII LGLPPCLNIL
RRKQPQLTFF TIALQSCHYQ RLPPHILWAT GLKSGGSSGG SSGSETPGTS
ESATPESSGG SSGGSDKKYS IGLAIGTNSV GWAVITDEYK VPSKKFKVLG
NTDRHSIKKN LIGALLFDSG ETAEATRLKR TARRRYTRRK NRICYLQEIF
SNEMAKVDDS FFHRLEESFL VEEDKKHERH PIFGNIVDEV AYHEKYPTIY
HLRKKLVDST DKADLRLIYL ALAHMIKFRG HFLIEGDLNP DNSDVDKLFI
QLVQTYNQLF EENPINASGV DAKAILSARL SKSRRLENLI AQLPGEKKNG
LFGNLIALSL GLTPNFKSNF DLAEDAKLQL SKDTYDDDLD NLLAQIGDQY
ADLFLAAKNL SDAILLSDIL RVNTEITKAP LSASMIKRYD EHHQDLTLLK
ALVRQQLPEK YKEIFFDQSK NGYAGYIDGG ASQEEFYKFI KPILEKMDGT
EELLVKLNRE DLLRKQRTFD NGSIPHQIHL GELHAILRRQ EDFYPFLKDN
REKIEKILTF RIPYYVGPLA RGNSRFAWMT RKSEETITPW NFEEVVDKGA
SAQSFIERMT NFDKNLPNEK VLPKHSLLYE YFTVYNELTK VKYVTEGMRK
PAFLSGEQKK AIVDLLFKTN RKVTVKQLKE DYFKKIECFD SVEISGVEDR
FNASLGTYHD LLKIIKDKDF LDNEENEDIL EDIVLTLTLF EDREMIEERL
KTYAHLFDDK VMKQLKRRRY TGWGRLSRKL INGIRDKQSG KTILDFLKSD
GFANRNFMQL IHDDSLTFKE DIQKAQVSGQ GDSLHEHIAN LAGSPAIKKG
ILQTVKVVDE LVKVMGRHKP ENIVIEMARE NQTTQKGQKN SRERMKRIEE
GIKELGSQIL KEHPVENTQL QNEKLYLYYL QNGRDMYVDQ ELDINRLSDY
DVDHIVPQSF LKDDSIDNKV LTRSDKNRGK SDNVPSEEVV KKMKNYWRQL
LNAKLITQRK FDNLTKAERG GLSELDKAGF IKRQLVETRQ ITKHVAQILD
SRMNTKYDEN DKLIREVKVI TLKSKLVSDF RKDFQFYKVR EINNYHHAHD
AYLNAVVGTA LIKKYPKLES EFVYGDYKVY DVRKMIAKSE QEIGKATAKY
FFYSNIMNFF KTEITLANGE IRKRPLIETN GETGEIVWDK GRDFATVRKV
LSMPQVNIVK KTEVQTGGFS KESILPKRNS DKLIARKKDW DPKKYGGFDS
PTVAYSVLVV AKVEKGKSKK LKSVKELLGI TIMERSSFEK NPIDFLEAKG
YKEVKKDLII KLPKYSLFEL ENGRKRMLAS AGELQKGNEL ALPSKYVNFL
YLASHYEKLK GSPEDNEQKQ LFVEQHKHYL DEIIEQISEF SKRVILADAN
LDKVLSAYNK HRDKPIREQA ENIIHLFTLT NLGAPAAFKY FDTTIDRKRY
TSTKEVLDAT LIHQSITGLY ETRIDLSQLG GDSGGSGGSG GSTNLSDIIE
KETGKQLVIQ ESILMLPEEV EEVIGNKPES DILVHTAYDE STDENVMLLT
SDAPEYKPWA LVIQDSNGEN KIKMLSGGSG GSGGSTNLSD IIEKETGKQL
VIQESILMLP EEVEEVIGNK PESDILVHTA YDESTDENVM LLTSDAPEYK
PWALVIQDSN GENKIKMLSG GSPKKKRK
Exemplary BE4 sequence with His tag:
(SEQ ID NO: 689)
MHHHHHHHHGSGGSGSSETGPVAVDPTLRRRIEPHEFEVFFDPRELRKET
CLLYEINWGGRHSIWRHTSQ NTNKHVEVNF IEKFTTERYF CPNTRCSITW
FLSWSPCGEC SRAITEFLSRYPHVTLFIYI ARLYHHADPR NRQGLRDLIS
SGVTIQIMTE QESGYCWRNF VNYSPSNEAH WPRYPHLWVR LYVLELYCII
LGLPPCLNIL RRKQPQLTFF TIALQSCHYQ RLPPHILWAT GLKSGGSSGG
SSGSETPGTS ESATPESSGG SSGGSDKKYS IGLAIGTNSV GWAVITDEYK
VPSKKFKVLG NTDRHSIKKN LIGALLFDSG ETAEATRLKR TARRRYTRRK
NRICYLQEIF SNEMAKVDDS FFHRLEESFL VEEDKKHERH PIFGNIVDEV
AYHEKYPTIY HLRKKLVDST DKADLRLIYL ALAHMIKFRG HFLIEGDLNP
DNSDVDKLFI QLVQTYNQLF EENPINASGV DAKAILSARL SKSRRLENLI
AQLPGEKKNG LFGNLIALSL GLTPNFKSNF DLAEDAKLQL SKDTYDDDLD
NLLAQIGDQY ADLFLAAKNL SDAILLSDIL RVNTEITKAP LSASMIKRYD
EHHQDLTLLK ALVRQQLPEK YKEIFFDQSK NGYAGYIDGG ASQEEFYKFI
KPILEKMDGT EELLVKLNRE DLLRKQRTFD NGSIPHQIHL GELHAILRRQ
EDFYPFLKDN REKIEKILTF RIPYYVGPLA RGNSRFAWMT RKSEETITPW
NFEEVVDKGA SAQSFIERMT NFDKNLPNEK VLPKHSLLYE YFTVYNELTK
VKYVTEGMRK PAFLSGEQKK AIVDLLFKTN RKVTVKQLKE DYFKKIECFD
SVEISGVEDR FNASLGTYHD LLKIIKDKDF LDNEENEDIL EDIVLTLTLF
EDREMIEERL KTYAHLFDDK VMKQLKRRRY TGWGRLSRKL INGIRDKQSG
KTILDFLKSD GFANRNFMQL IHDDSLTFKE DIQKAQVSGQ GDSLHEHIAN
LAGSPAIKKG ILQTVKVVDE LVKVMGRHKP ENIVIEMARE NQTTQKGQKN
SRERMKRIEE GIKELGSQIL KEHPVENTQL QNEKLYLYYL QNGRDMYVDQ
ELDINRLSDY DVDHIVPQSF LKDDSIDNKV LTRSDKNRGK SDNVPSEEVV
KKMKNYWRQL LNAKLITQRK FDNLTKAERG GLSELDKAGF IKRQLVETRQ
ITKHVAQILD SRMNTKYDEN DKLIREVKVI TLKSKLVSDF RKDFQFYKVR
EINNYHHAHD AYLNAVVGTA LIKKYPKLES EFVYGDYKVY DVRKMIAKSE
QEIGKATAKY FFYSNIMNFF KTEITLANGE IRKRPLIETN GETGEIVWDK
GRDFATVRKV LSMPQVNIVK KTEVQTGGFS KESILPKRNS DKLIARKKDW
DPKKYGGFDS PTVAYSVLVV AKVEKGKSKK LKSVKELLGI TIMERSSFEK
NPIDFLEAKG YKEVKKDLII KLPKYSLFEL ENGRKRMLAS AGELQKGNEL
ALPSKYVNFL YLASHYEKLK GSPEDNEQKQ LFVEQHKHYL DEIIEQISEF
SKRVILADAN LDKVLSAYNK HRDKPIREQA ENIIHLFTLT NLGAPAAFKY
FDTTIDRKRY TSTKEVLDAT LIHQSITGLY ETRIDLSQLG GDSGGSGGSG
GSTNLSDIIE KETGKQLVIQ ESILMLPEEV EEVIGNKPES DILVHTAYDE
STDENVMLLT SDAPEYKPWA LVIQDSNGEN KIKMLSGGSG GSGGSTNLSD
IIEKETGKQL VIQESILMLP EEVEEVIGNK PESDILVHTA YDESTDENVM
LLTSDAPEYK PWALVIQDSN GENKIKMLSG GSPKKKRK
Rat APOBEC1
(SEQ ID NO: 203)
SSETGPVAVDPTLRRRIEPHEFEVFFDPRELRKETCLLYEINWGGRHSIWRHTSQNTN
KHVEVNFIEKFTTERYFCPNTRCSITWFLSWSPCGECSRAITEFLSRYPHVTLFIYIARL
YHHADPRNRQGLRDLISSGVTIQIMTEQESGYCWRNFVNYSPSNEAHWPRYPHLWV
RLYVLELYCIILGLPPCLNILRRKQPQLTFFTIALQSCHYQRLPPH ILWATGLK
Anc689 APOBEC
(SEQ ID NO: 204)
SSETGPVAVDPTLRRRIEPHEFEVFFDPRELRKETCLLYEIKWGTSHKIWRHSSKNTT
KHVEVNFIEKFTSERHFCPSTSCSITWFLSWSPCGECSKAITEFLSQHPNVTLVIYVAR
LYHHMDQQNRQGLRDLVNSGVTIQIMTAPEYDYCWRNFVNYPPGKEAHWPRYPPL
WMKLYALELHAGILGLPPCLNILRRKQPQLTFFTIALQSCHYQRLP PHILWATGLK
Anc687 APOBEC
(SEQ ID NO: 205)
SSETGPVAVDPTLRRRIEPHEFEVFFDPRELRKEACLLYEIKWGTSHKIWRNSGKNTT
KHVEVNFIEKFTSERHFCPSISCSITWFLSWSPCWECSKAIREFLSQHPNVTLVIYVAR
LFQHMDQQNRQGLRDLVNSGVTIQIMTASEYDHCWRNFVNYPPGKEAHWPRYPPL
WMKLYALELHAGILGLPPCLNILRRKQPQLTFFTIALQSCHYQRLP PHILWATGLK
Anc686 APOBEC
(SEQ ID NO: 206)
SSETGPVAVDPTLRRRIEPEFFNRNYDPRELRKETYLLYEIKWGKESKI
WRHTSNNRTQHAEVNFLENFFNELYFNPSTHCSITWFLSWSPCGECSKAIVEFLKEHP
NVNLEIYVARLYLCEDERNRQGLRDLVNSGVTIRIMNLPDYNYCWRTFVSHQGGDE
DYWPRHFAPWVRLYVLELYCIILGLPPCLNILRRKQPQLTFFTIALQSCHYQRLP
PHILWATGLK
Anc655 APOBEC
(SEQ ID NO: 207)
SSETGPVAVDPTLRRRIEPFYFQFNNDPRACRRKTYLCYELKQDGSTWVWKRTLHN
KGRHAEICFLEKISSLEKLDPAQHYRITWYMSWSPCSNCAQKIVDFLKEHPHVNLRIY
VARLYYHEEERYQEGLRNLRRSGVSIRVMDLPDFEHCWETFVDNGGGPFQPWPGLE
ELNSKQLSRRLQAGILGLPPCLNILRRKQPQLTFFTIALQSCHYQR LPPHILWATGLK
Anc733 APOBEC
(SEQ ID NO: 208)
SSETGPVAVDPTLRRRIEPFHFQFNNDPRAYRRKTYLCYELKQDGSTWVLDRTLRNK
GRHAEICFLDKINSWERLDPAQHYRVTWYMSWSPCSNCAQQVVDFLKEHPHVNLRI
FAARLYYHEQRRYQEGLRSLRGSGVPVAVMTLPDFEHCWETFVDHGGRPFQPWDG
LEELNSRSLSRRLQAGILGLPPCLNILRRKQPQLTFFTIALQSCHY
QRLPPHILWATGLK
P2A-GFP
(SEQ ID NO: 209)
GSGATNFSLLKQAGDVEENPGP_MVSKGEELFTGVVPILVELDGDVNGHKFSVSGEG
EGDATYGKLTLKFICTTGKLPVPWPTLVTTLTYGVQCFSRYPDHMKQHDFFKSAMP
EGYVQERTIFFKDDGNYKTRAEVKFEGDTLVNRIELKGIDFKEDGNILGHKLEYNYN
SHNVYIMADKQKNGIKVNFKIRHNIEDGSVQLADHYQQNTPIGDGPVLLPDNHYLST
QSALSKDPNEKRDHMVLLEFVTAAGITLGMDELYKSGGSPKKKRKV
Exemplary ABE7.10 with His tag:
(SEQ ID NO: 690)
MHHHHHHHHG SGGSGSEVEF SHEYWMRHAL TLAKRAWDER EVPVGAVLVH
NNRVIGEGWN RPIGRHDPTA HAEIMALRQG GLVMQNYRLI DATLYVTLEP
CVMCAGAMIH SRIGRVVFGA RDAKTGAAGS LMDVLHHPGM NHRVEITEGI
LADECAALLS DFFRMRRQEI KAQKKAQSST DSGGSSGGSS GSETPGTSES
ATPESSGGSS GGSSEVEFSH EYWMRHALTL AKRARDEREV PVGAVLVLNN
RVIGEGWNRA IGLHDPTAHA EIMALRQGGL VMQNYRLIDA TLYVTFEPCV
MCAGAMIHSR IGRVVFGVRN AKTGAAGSLM DVLHYPGMNH RVEITEGILA
DECAALLCYF FRMPRQVFNA QKKAQSSTDS GGSSGGSSGS ETPGTSESAT
PESSGGSSGG SDKKYSIGLA IGTNSVGWAV ITDEYKVPSK KFKVLGNTDR
HSIKKNLIGA LLFDSGETAE ATRLKRTARR RYTRRKNRIC YLQEIFSNEM
AKVDDSFFHR LEESFLVEED KKHERHPIFG NIVDEVAYHE KYPTIYHLRK
KLVDSTDKAD LRLIYLALAH MIKFRGHFLI EGDLNPDNSD VDKLFIQLVQ
TYNQLFEENP INASGVDAKA ILSARLSKSR RLENLIAQLP GEKKNGLFGN
LIALSLGLTP NFKSNFDLAE DAKLQLSKDT YDDDLDNLLA QIGDQYADLF
LAAKNLSDAI LLSDILRVNT EITKAPLSAS MIKRYDEHHQ DLTLLKALVR
QQLPEKYKEI FFDQSKNGYA GYIDGGASQE EFYKFIKPIL EKMDGTEELL
VKLNREDLLR KQRTFDNGSI PHQIHLGELH AILRRQEDFY PFLKDNREKI
EKILTFRIPY YVGPLARGNS RFAWMTRKSE ETITPWNFEE VVDKGASAQS
FIERMTNFDK NLPNEKVLPK HSLLYEYFTV YNELTKVKYV TEGMRKPAFL
SGEQKKAIVD LLFKTNRKVT VKQLKEDYFK KIECFDSVEI SGVEDRENAS
LGTYHDLLKI IKDKDFLDNE ENEDILEDIV LTLTLFEDRE MIEERLKTYA
HLFDDKVMKQ LKRRRYTGWG RLSRKLINGI RDKQSGKTIL DFLKSDGFAN
RNFMQLIHDD SLTFKEDIQK AQVSGQGDSL HEHIANLAGS PAIKKGILQT
VKVVDELVKV MGRHKPENIV IEMARENQTT QKGQKNSRER MKRIEEGIKE
LGSQILKEHP VENTQLQNEK LYLYYLQNGR DMYVDQELDI NRLSDYDVDH
IVPQSFLKDD SIDNKVLTRS DKNRGKSDNV PSEEVVKKMK NYWRQLLNAK
LITQRKFDNL TKAERGGLSE LDKAGFIKRQ LVETRQITKH VAQILDSRMN
TKYDENDKLI REVKVITLKS KLVSDFRKDF QFYKVREINN YHHAHDAYLN
AVVGTALIKK YPKLESEFVY GDYKVYDVRK MIAKSEQEIG KATAKYFFYS
NIMNFFKTEI TLANGEIRKR PLIETNGETG EIVWDKGRDF ATVRKVLSMP
QVNIVKKTEV QTGGFSKESI LPKRNSDKLI ARKKDWDPKK YGGFDSPTVA
YSVLVVAKVE KGKSKKLKSV KELLGITIME RSSFEKNPID FLEAKGYKEV
KKDLIIKLPK YSLFELENGR KRMLASAGEL QKGNELALPS KYVNFLYLAS
HYEKLKGSPE DNEQKQLFVE QHKHYLDEII EQISEFSKRV ILADANLDKV
LSAYNKHRDK PIREQAENII HLFTLTNLGA PAAFKYFDTT IDRKRYTSTK
EVLDATLIHQ SITGLYETRI DLSQLGGDSG GSPKKKRKV
Exemplary ABE:
(SEQ ID NO: 691)
MSEVEF SHEYWMRHAL TLAKRAWDER EVPVGAVLVH NNRVIGEGWN
RPIGRHDPTA HAEIMALRQG GLVMQNYRLI DATLYVTLEP CVMCAGAMIH
SRIGRVVFGA RDAKTGAAGS LMDVLHHPGM NHRVEITEGI LADECAALLS
DFFRMRRQEI KAQKKAQSST DSGGSSGGSS GSETPGTSES ATPESSGGSS
GGSSEVEFSH EYWMRHALTL AKRARDEREV PVGAVLVLNN RVIGEGWNRA
IGLHDPTAHA EIMALRQGGL VMQNYRLIDA TLYVTFEPCV MCAGAMIHSR
IGRVVFGVRN AKTGAAGSLM DVLHYPGMNH RVEITEGILA DECAALLCYF
FRMPRQVFNA QKKAQSSTDS GGSSGGSSGS ETPGTSESAT PESSGGSSGG
SDKKYSIGLA IGTNSVGWAV ITDEYKVPSK KFKVLGNTDR HSIKKNLIGA
LLFDSGETAE ATRLKRTARR RYTRRKNRIC YLQEIFSNEM AKVDDSFFHR
LEESFLVEED KKHERHPIFG NIVDEVAYHE KYPTIYHLRK KLVDSTDKAD
LRLIYLALAH MIKFRGHFLI EGDLNPDNSD VDKLFIQLVQ TYNQLFEENP
INASGVDAKA ILSARLSKSR RLENLIAQLP GEKKNGLFGN LIALSLGLTP
NFKSNFDLAE DAKLQLSKDT YDDDLDNLLA QIGDQYADLF LAAKNLSDAI
LLSDILRVNT EITKAPLSAS MIKRYDEHHQ DLTLLKALVR QQLPEKYKEI
FFDQSKNGYA GYIDGGASQE EFYKFIKPIL EKMDGTEELL VKLNREDLLR
KQRTFDNGSI PHQIHLGELH AILRRQEDFY PFLKDNREKI EKILTFRIPY
YVGPLARGNS RFAWMTRKSE ETITPWNFEE VVDKGASAQS FIERMTNFDK
NLPNEKVLPK HSLLYEYFTV YNELTKVKYV TEGMRKPAFL SGEQKKAIVD
LLFKTNRKVT VKQLKEDYFK KIECFDSVEI SGVEDRFNAS LGTYHDLLKI
IKDKDFLDNE ENEDILEDIV LTLTLFEDRE MIEERLKTYA HLFDDKVMKQ
LKRRRYTGWG RLSRKLINGI RDKQSGKTIL DFLKSDGFAN RNFMQLIHDD
SLTFKEDIQK AQVSGQGDSL HEHIANLAGS PAIKKGILQT VKVVDELVKV
MGRHKPENIV IEMARENQTT QKGQKNSRER MKRIEEGIKE LGSQILKEHP
VENTQLQNEK LYLYYLQNGR DMYVDQELDI NRLSDYDVDH IVPQSFLKDD
SIDNKVLTRS DKNRGKSDNV PSEEVVKKMK NYWRQLLNAK LITQRKFDNL
TKAERGGLSE LDKAGFIKRQ LVETRQITKH VAQILDSRMN TKYDENDKLI
REVKVITLKS KLVSDFRKDF QFYKVREINN YHHAHDAYLN AVVGTALIKK
YPKLESEFVY GDYKVYDVRK MIAKSEQEIG KATAKYFFYS NIMNFFKTEI
TLANGEIRKR PLIETNGETG EIVWDKGRDF ATVRKVLSMP QVNIVKKTEV
QTGGFSKESI LPKRNSDKLI ARKKDWDPKK YGGFDSPTVA YSVLVVAKVE
KGKSKKLKSV KELLGITIME RSSFEKNPID FLEAKGYKEV KKDLIIKLPK
YSLFELENGR KRMLASAGEL QKGNELALPS KYVNFLYLAS HYEKLKGSPE
DNEQKQLFVE QHKHYLDEII EQISEFSKRV ILADANLDKV LSAYNKHRDK
PIREQAENII HLFTLTNLGA PAAFKYFDTT IDRKRYTSTK EVLDATLIHQ
SITGLYETRI DLSQLGGDSG GSPKKKRKV
ABEmax
(SEQ ID NO: 210)
MKRTADGSEFESPKKKRKV_MSEVEFSHEYWMRHALTLAKRAWDEREVPVGAVLVH
NNRVIGEGWNRPIGRHDPTAHAEIMALRQGGLVMQNYRLIDATLYVTLEPCVMCAGAMIH
SRIGRVVFGARDAKTGAAGSLMDVLHHPGMNHRVEITEGILADECAALLSDFFRMRRQEI
KAQKKAQSSTD_SGGSSGGSSGSETPGTSESATPESSGGSSGGS_SEVEFSHEYWMRHAL
TLAKRARDEREVPVGAVLVLNNRVIGEGWNRAIGLHDPTAHAEIMALRQGGLVMQNYRLI
DATLYVTFEPCVMCAGAMIHSRIGRVVFGVRNAKTGAAGSLMDVLHYPGMNHRVEITEGI
LADECAALLCYFFRMPRQVFNAQKKAQSSTD_SGGSSGGSSGSETPGTSESATPESSGGS
SGGS_DKKYSIGLAIGTNSVGWAVITDEYKVPSKKFKVLGNTDRHSIKKNLIGALLFD
SGETAEATRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHMIKFRGHFL
IEGDLNPDNSDVDKLFIQLVQTYNQLFEENPINASGVDAKAILSARLSKSRRLENLIAQ
LPGEKKNGLFGNLIALSLGLTPNFKSNFDLAEDAKLQLSKDTYDDDLDNLLAQIGDQ
YADLFLAAKNLSDAILLSDILRVNTEITKAPLSASMIKRYDEHHQDLTLLKALVRQQL
PEKYKEIFFDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYYVGPLARGNS
RFAWMTRKSEETITPWNFEEVVDKGASAQSFIERMTNFDKNLPNEKVLPKHSLLYEY
FTVYNELTKVKYVTEGMRKPAFLSGEQKKAIVDLLFKTNRKVTVKQLKEDYFKKIE
CFDSVEISGVEDRENASLGTYHDLLKIIKDKDFLDNEENEDILEDIVLTLTLFEDREMIE
ERLKTYAHLEDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQTVKVVDE
LVKVMGRHKPENIVIEMARENQTTQKGQKNSRERMKRIEEGIKELGSQILKEHPVEN
TQLQNEKLYLYYLQNGRDMYVDQELDINRLSDYDVDHIVPQSFLKDDSIDNKVLTR
SDKNRGKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVSDFRKDFQF
YKVREINNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKGRDFATVRK
VLSMPQVNIVKKTEVQTGGFSKESILPKRNSDKLIARKKDWDPKKYGGFDSPTVAYS
VLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQ
LFVEQHKHYLDEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLGGD_KRTAD
GSEFEPKKKRKV[optional P2A-GFP]

Sequences 4. Codon Optimized Nucleotide Sequences of BE4, rAPOBEC, Ancestral APOBECs, ABE, and P2A GFP.

Exemplary ABEmax:
(SEQ ID NO: 694)
MKRTADGSEFESPKKKRKVSEVEFSHEYWMRHALTLAKRAWDEREVPVGA
VLVHNNRVIGEGWNRPIGRHDPTAHAEIMALRQGGLVMQNYRLIDATLYV
TLEPCVMCAGAMIHSRIGRVVFGARDAKTGAAGSLMDVLHHPGMNHRVEI
TEGILADECAALLSDFFRMRRQEIKAQKKAQSSTDSGGSSGGSSGSETPG
TSESATPESSGGSSGGSSEVEFSHEYWMRHALTLAKRARDEREVPVGAVL
VLNNRVIGEGWNRAIGLHDPTAHAEIMALRQGGLVMQNYRLIDATLYVTF
EPCVMCAGAMIHSRIGRVVFGVRNAKTGAAGSLMDVLHYPGMNHRVEITE
GILADECAALLCYFFRMPRQVFNAQKKAQSSTDSGGSSGGSSGSETPGTS
ESATPESSGGSSGGSDKKYSIGLAIGTNSVGWAVITDEYKVPSKKFKVLG
NTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQY
ADLFLAAKNLSDAILLSDILRVNTEITKAPLSASMIKRYDEHHQDLTLLK
ALVRQQLPEKYKEIFFDQSKNGYAGYIDGGASQEEFYKFIKPILEKMDGT
EELLVKLNREDLLRKQRTFDNGSIPHQIHLGELHAILRRQEDFYPFLKDN
REKIEKILTFRIPYYVGPLARGNSRFAWMTRKSEETITPWNFEEVVDKGA
SAQSFIERMTNFDKNLPNEKVLPKHSLLYEYFTVYNELTKVKYVTEGMRK
PAFLSGEQKKAIVDLLFKTNRKVTVKQLKEDYFKKIECFDSVEISGVEDR
FNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKG
ILQTVKVVDELVKVMGRHKPENIVIEMARENQTTQKGQKNSRERMKRIEE
GIKELGSQILKEHPVENTQLQNEKLYLYYLQNGRDMYVDQELDINRLSDY
DVDHIVPQSFLKDDSIDNKVLTRSDKNRGKSDNVPSEEVVKKMKNYWRQL
LNAKLITQRKFDNLTKAERGGLSELDKAGFIKRQLVETRQITKHVAQILD
SRMNTKYDENDKLIREVKVITLKSKLVSDFRKDFQFYKVREINNYHHAHD
AYLNAVVGTALIKKYPKLESEFVYGDYKVYDVRKMIAKSEQEIGKATAKY
FFYSNIMNFFKTEITLANGEIRKRPLIETNGETGEIVWDKGRDFATVRKV
LSMPQVNIVKKTEVQTGGFSKESILPKRNSDKLIARKKDWDPKKYGGFDS
PTVAYSVLVVAKVEKGKSKKLKSVKELLGITIMERSSFEKNPIDFLEAKG
YKEVKKDLIIKLPKYSLFELENGRKRMLASAGELQKGNELALPSKYVNFL
YLASHYEKLKGSPEDNEQKQLFVEQHKHYLDEIIEQISEFSKRVILADAN
LDKVLSAYNKHRDKPIREQAENIIHLFTLTNLGAPAAFKYFDTTIDRKRY
TSTKEVLDATLIHQSITGLYETRIDLSQLGGDSGGSKRTADGSEFEPKKK
RKV

Within base editor sequences. NLS sequences are bold. APOBEC and TadA sequences are italicized, linkers are double underlined. Cas9 nickase sequence is underlined, and UGI sequences are bold and italicized.

BE4max and AncBE4max
(SEQ ID NO: 211)
ATGAAACGGACAGCCGACGGAAGCGAGTTCGAGTCACCAAAGAAGAAGCGG
AAAGTC_APOBEC_TCTGGAGGATCTAGCGGAGGATCCTCTGGCAGCGAGACACC
AGGAACAAGCGAGTCAGCAACACCAGAGAGCAGTGGCGGCAGCAGCGGCGGCA
GC_GACAAGAAGTACAGCATCGGCCTGGACATCGGCACCAACTCTGTGGGCTGG
GCCGTGATCACCGACGAGTACAAGGTGCCCAGCAAGAAATTCAAGGTGCTGGGC
AACACCGACCGGCACAGCATCAAGAAGAACCTGATCGGAGCCCTGCTGTTCGAC
AGCGGCGAAACAGCCGAGGCCACCCGGCTGAAGAGAACCGCCAGAAGAAGATA
CACCAGACGGAAGAACCGGATCTGCTATCTGCAAGAGATCTTCAGCAACGAGAT
GGCCAAGGTGGACGACAGCTTCTTCCACAGACTGGAAGAGTCCTTCCTGGTGGA
AGAGGATAAGAAGCACGAGCGGCACCCCATCTTCGGCAACATCGTGGACGAGGT
GGCCTACCACGAGAAGTACCCCACCATCTACCACCTGAGAAAGAAACTGGTGGA
CAGCACCGACAAGGCCGACCTGCGGCTGATCTATCTGGCCCTGGCCCACATGATC
AAGTTCCGGGGCCACTTCCTGATCGAGGGCGACCTGAACCCCGACAACAGCGAC
GTGGACAAGCTGTTCATCCAGCTGGTGCAGACCTACAACCAGCTGTTCGAGGAA
AACCCCATCAACGCCAGCGGCGTGGACGCCAAGGCCATCCTGTCTGCCAGACTG
AGCAAGAGCAGACGGCTGGAAAATCTGATCGCCCAGCTGCCCGGCGAGAAGAA
GAATGGCCTGTTCGGAAACCTGATTGCCCTGAGCCTGGGCCTGACCCCCAACTTC
AAGAGCAACTTCGACCTGGCCGAGGATGCCAAACTGCAGCTGAGCAAGGACACC
TACGACGACGACCTGGACAACCTGCTGGCCCAGATCGGCGACCAGTACGCCGAC
CTGTTTCTGGCCGCCAAGAACCTGTCCGACGCCATCCTGCTGAGCGACATCCTGA
GAGTGAACACCGAGATCACCAAGGCCCCCCTGAGCGCCTCTATGATCAAGAGAT
ACGACGAGCACCACCAGGACCTGACCCTGCTGAAAGCTCTCGTGCGGCAGCAGC
TGCCTGAGAAGTACAAAGAGATTTTCTTCGACCAGAGCAAGAACGGCTACGCCG
GCTACATTGACGGCGGAGCCAGCCAGGAAGAGTTCTACAAGTTCATCAAGCCCA
TCCTGGAAAAGATGGACGGCACCGAGGAACTGCTCGTGAAGCTGAACAGAGAGG
ACCTGCTGCGGAAGCAGCGGACCTTCGACAACGGCAGCATCCCCCACCAGATCC
ACCTGGGAGAGCTGCACGCCATTCTGCGGCGGCAGGAAGATTTTTACCCATTCCT
GAAGGACAACCGGGAAAAGATCGAGAAGATCCTGACCTTCCGCATCCCCTACTA
CGTGGGCCCTCTGGCCAGGGGAAACAGCAGATTCGCCTGGATGACCAGAAAGAG
CGAGGAAACCATCACCCCCTGGAACTTCGAGGAAGTGGTGGACAAGGGCGCTTC
CGCCCAGAGCTTCATCGAGCGGATGACCAACTTCGATAAGAACCTGCCCAACGA
GAAGGTGCTGCCCAAGCACAGCCTGCTGTACGAGTACTTCACCGTGTATAACGA
GCTGACCAAAGTGAAATACGTGACCGAGGGAATGAGAAAGCCCGCCTTCCTGAG
CGGCGAGCAGAAAAAGGCCATCGTGGACCTGCTGTTCAAGACCAACCGGAAAGT
GACCGTGAAGCAGCTGAAAGAGGACTACTTCAAGAAAATCGAGTGCTTCGACTC
CGTGGAAATCTCCGGCGTGGAAGATCGGTTCAACGCCTCCCTGGGCACATACCA
CGATCTGCTGAAAATTATCAAGGACAAGGACTTCCTGGACAATGAGGAAAACGA
GGACATTCTGGAAGATATCGTGCTGACCCTGACACTGTTTGAGGACAGAGAGAT
GATCGAGGAACGGCTGAAAACCTATGCCCACCTGTTCGACGACAAAGTGATGAA
GCAGCTGAAGCGGCGGAGATACACCGGCTGGGGCAGGCTGAGCCGGAAGCTGAT
CAACGGCATCCGGGACAAGCAGTCCGGCAAGACAATCCTGGATTTCCTGAAGTC
CGACGGCTTCGCCAACAGAAACTTCATGCAGCTGATCCACGACGACAGCCTGAC
CTTTAAAGAGGACATCCAGAAAGCCCAGGTGTCCGGCCAGGGCGATAGCCTGCA
CGAGCACATTGCCAATCTGGCCGGCAGCCCCGCCATTAAGAAGGGCATCCTGCA
GACAGTGAAGGTGGTGGACGAGCTCGTGAAAGTGATGGGCCGGCACAAGCCCGA
GAACATCGTGATCGAAATGGCCAGAGAGAACCAGACCACCCAGAAGGGACAGA
AGAACAGCCGCGAGAGAATGAAGCGGATCGAAGAGGGCATCAAAGAGCTGGGC
AGCCAGATCCTGAAAGAACACCCCGTGGAAAACACCCAGCTGCAGAACGAGAA
GCTGTACCTGTACTACCTGCAGAATGGGCGGGATATGTACGTGGACCAGGAACT
GGACATCAACCGGCTGTCCGACTACGATGTGGACCATATCGTGCCTCAGAGCTTT
CTGAAGGACGACTCCATCGACAACAAGGTGCTGACCAGAAGCGACAAGAACCG
GGGCAAGAGCGACAACGTGCCCTCCGAAGAGGTCGTGAAGAAGATGAAGAACT
ACTGGCGGCAGCTGCTGAACGCCAAGCTGATTACCCAGAGAAAGTTCGACAATC
TGACCAAGGCCGAGAGAGGCGGCCTGAGCGAACTGGATAAGGCCGGCTTCATCA
AGAGACAGCTGGTGGAAACCCGGCAGATCACAAAGCACGTGGCACAGATCCTGG
ACTCCCGGATGAACACTAAGTACGACGAGAATGACAAGCTGATCCGGGAAGTGA
AAGTGATCACCCTGAAGTCCAAGCTGGTGTCCGATTTCCGGAAGGATTTCCAGTT
TTACAAAGTGCGCGAGATCAACAACTACCACCACGCCCACGACGCCTACCTGAA
CGCCGTCGTGGGAACCGCCCTGATCAAAAAGTACCCTAAGCTGGAAAGCGAGTT
CGTGTACGGCGACTACAAGGTGTACGACGTGCGGAAGATGATCGCCAAGAGCGA
GCAGGAAATCGGCAAGGCTACCGCCAAGTACTTCTTCTACAGCAACATCATGAA
CTTTTTCAAGACCGAGATTACCCTGGCCAACGGCGAGATCCGGAAGCGGCCTCTG
ATCGAGACAAACGGCGAAACCGGGGAGATCGTGTGGGATAAGGGCCGGGATTTT
GCCACCGTGCGGAAAGTGCTGAGCATGCCCCAAGTGAATATCGTGAAAAAGACC
GAGGTGCAGACAGGCGGCTTCAGCAAAGAGTCTATCCTGCCCAAGAGGAACAGC
GATAAGCTGATCGCCAGAAAGAAGGACTGGGACCCTAAGAAGTACGGCGGCTTC
GACAGCCCCACCGTGGCCTATTCTGTGCTGGTGGTGGCCAAAGTGGAAAAGGGC
AAGTCCAAGAAACTGAAGAGTGTGAAAGAGCTGCTGGGGATCACCATCATGGAA
AGAAGCAGCTTCGAGAAGAATCCCATCGACTTTCTGGAAGCCAAGGGCTACAAA
GAAGTGAAAAAGGACCTGATCATCAAGCTGCCTAAGTACTCCCTGTTCGAGCTG
GAAAACGGCCGGAAGAGAATGCTGGCCTCTGCCGGCGAACTGCAGAAGGGAAA
CGAACTGGCCCTGCCCTCCAAATATGTGAACTTCCTGTACCTGGCCAGCCACTAT
GAGAAGCTGAAGGGCTCCCCCGAGGATAATGAGCAGAAACAGCTGTTTGTGGAA
CAGCACAAGCACTACCTGGACGAGATCATCGAGCAGATCAGCGAGTTCTCCAAG
AGAGTGATCCTGGCCGACGCTAATCTGGACAAAGTGCTGTCCGCCTACAACAAG
CACCGGGATAAGCCCATCAGAGAGCAGGCCGAGAATATCATCCACCTGTTTACC
CTGACCAATCTGGGAGCCCCTGCCGCCTTCAAGTACTTTGACACCACCATCGACC
GGAAGAGGTACACCAGCACCAAAGAGGTGCTGGACGCCACCCTGATCCACCAGA
GCATCACCGGCCTGTACGAGACACGGATCGACCTGTCTCAGCTGGGAGGTGAC_A
GCGGCGGGAGCGGCGGGAGCGGGGGGAGC_ACTAATCTGAGCGACATCATTGAG
AAGGAGACTGGGAAACAGCTGGTCATTCAGGAGTCCATCCTGATGCTGCCTGAGG
AGGTGGAGGAAGTGATCGGCAACAAGCCAGAGTCTGACATCCTGGTGCACACCGC
CTACGACGAGTCCACAGATGAGAATGTGATGCTGCTGACCTCTGACGCCCCCGAGT
ATAAGCCTTGGGCCCTGGTCATCCAGGATTCTAACGGCGAGAATAAGATCAAGATG
CTG_AGCGGAGGATCCGGAGGATCTGGAGGCAGC_ACCAACCTGTCTGACATCAT
CGAGAAGGAGACAGGCAAGCAGCTGGTCATCCAGGAGAGCATCCTGATGCTGCCC
GAAGAAGTCGAAGAAGTGATCGGAAACAAGCCTGAGAGCGATATCCTGGTCCATA
CCGCCTACGACGAGAGTACCGACGAAAATGTGATGCTGCTGACATCCGACGCCCC
AGAGTATAAGCCCTGGGCTCTGGTCATCCAGGATTCCAACGGAGAGAACAAAATCA
AAATGCTG_TCTGGCGGCTCA_AAAAGAACCGCCGACGGCAGCGAATTCGAGC
CCAAGAAGAAGAGGAAAGTC_[optional P2A-GFP]_TAA

The portion indicated by APOBEC in the above sequence of SEQ ID NO 211 may include any nucleic acid sequence encoding an APOBEC, or variant thereof, provided herein. For example, in some embodiments, the APOBEC may comprise any one of SEQ ID NOs: 37-42 or 212-217.

Rat APOBEC1
(SEQ ID NO: 212)
TCCTCAGAGACTGGGCCTGTCGCCGTCGATCCAACCCTGCGCCGCCGGATTGAAC
CTCACGAGTTTGAAGTGTTCTTTGACCCCCGGGAGCTGAGAAAGGAGACATGCCT
GCTGTACGAGATCAACTGGGGAGGCAGGCACTCCATCTGGAGGCACACCTCTCA
GAACACAAATAAGCACGTGGAGGTGAACTTCATCGAGAAGTTTACCACAGAGCG
GTACTTCTGCCCCAATACCAGATGTAGCATCACATGGTTTCTGAGCTGGTCCCCTT
GCGGAGAGTGTAGCAGGGCCATCACCGAGTTCCTGTCCAGATATCCACACGTGA
CACTGTTTATCTACATCGCCAGGCTGTATCACCACGCAGACCCAAGGAATAGGCA
GGGCCTGCGCGATCTGATCAGCTCCGGCGTGACCATCCAGATCATGACAGAGCA
GGAGTCCGGCTACTGCTGGCGGAACTTCGTGAATTATTCTCCTAGCAACGAGGCC
CACTGGCCTAGGTACCCACACCTGTGGGTGCGCCTGTACGTGCTGGAGCTGTATT
GCATCATCCTGGGCCTGCCCCCTTGTCTGAATATCCTGCGGAGAAAGCAGCCCCA
GCTGACCTTCTTTACAATCGCCCTGCAGTCTTGTCACTATCAGAGGCTGCCACCC
CACATCCTGTGGGCCACAGGCCTGAAG
Anc689 APOBEC
(SEQ ID NO: 213)
AGCAGTGAAACCGGACCAGTGGCAGTGGACCCAACCCTGAGGAGACGGATTGAG
CCCCATGAATTTGAAGTGTTCTTTGACCCAAGGGAGCTGAGGAAGGAGACATGC
CTGCTGTACGAGATCAAGTGGGGCACAAGCCACAAGATCTGGCGCCACAGCTCC
AAGAACACCACAAAGCACGTGGAAGTGAATTTCATCGAGAAGTTTACCTCCGAG
CGGCACTTCTGCCCCTCTACCAGCTGTTCCATCACATGGTTTCTGTCTTGGAGCCC
TTGCGGCGAGTGTTCCAAGGCCATCACCGAGTTCCTGTCTCAGCACCCTAACGTG
ACCCTGGTCATCTACGTGGCCCGGCTGTATCACCACATGGACCAGCAGAACAGG
CAGGGCCTGCGCGATCTGGTGAATTCTGGCGTGACCATCCAGATCATGACAGCCC
CAGAGTACGACTATTGCTGGCGGAACTTCGTGAATTATCCACCTGGCAAGGAGG
CACACTGGCCAAGATACCCACCCCTGTGGATGAAGCTGTATGCACTGGAGCTGC
ACGCAGGAATCCTGGGCCTGCCTCCATGTCTGAATATCCTGCGGAGAAAGCAGC
CCCAGCTGACATTTTTCACCATTGCTCTGCAGTCTTGTCACTATCAGCGGCTGCCT
CCTCATATTCTGTGGGCTACAGGCCTTTGA AA
Anc687 APOBEC
(SEQ ID NO: 214)
TCATCAGAAACAGGACCAGTCGCCGTGGACCCAACACTGAGGAGAAGGATTGAG
CCCCATGAATTTGAAGTCTTTTTCGACCCCAGGGAGCTGAGGAAGGAGGCATGCC
TGCTGTACGAGATCAAGTGGGGCACAAGCCACAAGATCTGGCGCAACAGCGGCA
AGAACACCACAAAGCACGTGGAAGTGAATTTCATCGAGAAGTTTACCTCCGAGC
GGCACTTCTGCCCCTCTATCAGCTGTTCCATCACATGGTTTCTGTCTTGGAGCCCT
TGCTGGGAGTGTTCCAAGGCCATCCGCGAGTTCCTGTCTCAGCACCCTAACGTGA
CCCTGGTCATCTACGTGGCCCGGCTGTTTCAACACATGGACCAGCAGAACAGGC
AGGGCCTGCGCGATCTGGTGAATTCTGGCGTGACCATCCAGATCATGACAGCCTC
AGAGTACGACCATTGCTGGCGGAACTTCGTGAATTATCCACCTGGCAAGGAGGC
ACACTGGCCAAGATACCCACCCCTGTGGATGAAGCTGTATGCACTGGAGCTGCA
CGCAGGAATCCTGGGCCTGCCTCCATGTCTGAATATCCTGCGGAGAAAGCAGCC
CCAGCTGACATTTTTCACTATCGCACTGCAGAGCTGTCATTACCAGAGACTGCCT
CCTCATATCCTGTGGGCTACAGGCCTTTGAAA
Anc686 APOBEC
(SEQ ID NO: 215)
AGCAGCGAGACAGGACCCGTGGCAGTGGACCCTACACTGAGGAGGAGGATTGA
GCCCGAATTTTTCAACAGGAACTACGACCCCAGAGAGCTGCGGAAGGAGACATA
CCTGCTGTATGAGATCAAGTGGGGCAAGGAGTCCAAGATCTGGCGGCACACCTC
TAACAATAGAACACAGCACGCCGAGGTGAACTTCCTGGAGAACTTCTTTAATGA
GCTGTACTTTAATCCTTCTACCCACTGCAGCATCACATGGTTCCTGAGCTGGTCCC
CATGCGGCGAGTGTTCTAAGGCCATCGTGGAGTTTCTGAAGGAGCACCCCAACGT
GAATCTGGAGATCTACGTGGCCAGGCTGTATCTGTGCGAGGACGAGAGGAACAG
GCAGGGCCTGCGGGATCTGGTGAATAGCGGCGTGACCATCAGAATCATGAACCT
GCCTGACTACAATTATTGTTGGCGCACATTCGTGTCCCACCAGGGAGGCGACGAG
GATTATTGGCCAAGGCACTTTGCACCATGGGTGCGCCTGTACGTGCTGGAGCTGT
ATTGCATCATCCTGGGCCTGCCCCCTTGTCTGAACATCCTGCGGAGAAAGCAGCC
CCAGCTGACATTCTTCACCATCGCACTGCAGAGTTGTCATTACCAGCGACTGCCT
CCTCATATCCTGTGGGCTACAGGCCTTTGAAA
Anc655 APOBEC
(SEQ ID NO: 216)
TCATCAGAGACCGGACCTGTGGCAGTGGACCCAACCCTGCGACGGAGAATCGAG
CCCTTTTACTTTCAGTTCAACAACGACCCAAGAGCCTGCCGGAGAAAGACCTACC
TGTGCTATGAGCTGAAGCAGGACGGCTCTACCTGGGTGTGGAAGCGGACACTGC
ACAACAAGGGCAGACACGCCGAGATCTGCTTCCTGGAGAAGATCAGCTCCCTGG
AGAAGCTGGACCCTGCCCAGCACTACAGGATCACATGGTATATGTCTTGGAGCCC
ATGCTCCAACTGTGCCCAGAAGATCGTGGATTTTCTGAAGGAGCACCCACACGTG
AATCTGCGGATCTACGTGGCCAGACTGTACTATCACGAGGAGGAGAGGTATCAG
GAGGGCCTGAGGAACCTGAGGCGCTCCGGCGTGTCTATCAGAGTGATGGACCTG
CCCGATTTCGAGCACTGCTGGGAGACATTCGTGGATAACGGAGGAGGACCTTTCC
AGCCATGGCCCGGCCTGGAGGAGCTGAATAGCAAGCAGCTGTCCCGGAGACTGC
AGGCAGGAATCCTGGGCCTGCCCCCTTGTCTGAATATCCTGAGGCGCAAGCAGC
CCCAGCTGACATTTTTCACCATCGCACTGCAGAGTTGTCATTATCAGCGACTGCC
TCCTCATATCCTGTGGGCTACAGGCCTTTGAAA
Anc733 APOBEC
(SEQ ID NO: 217)
AGCAGCGAGACCGGACCTGTGGCAGTGGACCCAACCCTGAGAAGACGCATTGAG
CCATTTCATTTTCAGTTTAACAACGACCCCAGAGCCTACCGGAGAAAGACCTACC
TGTGCTATGAGCTGAAGCAGGACGGCTCCACCTGGGTGCTGGATCGGACACTGA
GAAACAAGGGCCGGCACGCCGAGATCTGTTTCCTGGACAAGATCAATTCCTGGG
AGAGGCTGGATCCCGCCCAGCACTACCGCGTGACATGGTATATGAGCTGGTCCC
CTTGCTCTAACTGTGCCCAGCAGGTGGTGGATTTCCTGAAGGAGCACCCACACGT
GAATCTGCGGATCTTTGCCGCCAGACTGTACTATCACGAGCAGAGGCGCTATCAG
GAGGGCCTGCGGAGCCTGAGGGGAAGCGGAGTGCCTGTGGCCGTGATGACCCTG
CCAGACTTCGAGCACTGCTGGGAGACATTTGTGGATCACGGCGGCCGGCCATTCC
AGCCATGGGACGGCCTGGAGGAGCTGAACTCTAGGAGCCTGTCCCGGAGACTGC
AGGCAGGAATCCTGGGCCTGCCCCCTTGTCTGAATATCCTGAGGCGCAAGCAGC
CCCAGCTGACCTTTTTTACCATCGCACTGCAGAGTTGTCACTACCAGAGACTGCC
TCCTCATATCCTGTGGGCTACAGGCCTTTGAAA
P2A-GFP
(SEQ ID NO: 218)
TCTGGTGGTTCTCCCAAGAAGAAGAGGAAAGTCGGAAGCGGAGCT
ACTAACTTCAGCCTGCTGAAGCAGGCTGGAGACGTGGAGGAGAACCCTGGACCT
ATGGTGAGCAAGGGCGAGGAGCTGTTCACCGGGGTGGTGCCCATCCTGGTCGAG
CTGGACGGCGACGTAAACGGCCACAAGTTCAGCGTGTCCGGCGAGGGCGAGGGC
GATGCCACCTACGGCAAGCTGACCCTGAAGTTCATCTGCACCACCGGCAAGCTG
CCCGTGCCCTGGCCCACCCTCGTGACCACCCTGACCTACGGCGTGCAGTGCTTCA
GCCGCTACCCCGACCACATGAAGCAGCACGACTTCTTCAAGTCCGCCATGCCCGA
AGGCTACGTCCAGGAGCGCACCATCTTCTTCAAGGACGACGGCAACTACAAGAC
CCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAA
CTACAACAGCCACAACGTCTATATCATGGCCGACAAGCAGAAGAACGGCATCAA
GGTGAACTTCAAGATCCGCCACAACATCGAGGACGGCAGCGTGCAGCTCGCCGA
CCACTACCAGCAGAACACCCCCATCGGCGACGGCCCCGTGCTGCTGCCCGACAA
CCACTACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCATGGAC
GAGCTGTACAAGTCTGGTGGTTCTCCCAAGAAGAAGAGGAAAGTCTAA
ABEmax
(SEQ ID NO: 219)
ATGAAACGGACAGCCGACGGAAGCGAGTTCGAGTCACCAAAGA
AGAAGCGGAAAGTC_TCTGAAGTCGAGTTTAGCCACGAGTATTGGATGAGGCACGC
ACTGACCCTGGCAAAGCGAGCATGGGATGAAAGAGAAGTCCCCGTGGGCGCCGTGCT
GGTGCACAACAATAGAGTGATCGGAGAGGGATGGAACAGGCCAATCGGCCGCCACGA
CCCTACCGCACACGCAGAGATCATGGCACTGAGGCAGGGAGGCCTGGTCATGCAGAA
TTACCGCCTGATCGATGCCACCCTGTATGTGACACTGGAGCCATGCGTGATGTGCGCA
GGAGCAATGATCCACAGCAGGATCGGAAGAGTGGTGTTCGGAGCACGGGACGCCAAG
ACCGGCGCAGCAGGCTCCCTGATGGATGTGCTGCACCACCCCGGCATGAACCACCGG
GTGGAGATCACAGAGGGAATCCTGGCAGACGAGTGCGCCGCCCTGCTGAGCGATTTC
TTTAGAATGCGGAGACAGGAGATCAAGGCCCAGAAGAAGGCACAGAGCTCCACCGAC
_TCTGGAGGATCTAGCGGAGGATCCTCTGGAAGCGAGACACCAGGCACAAGCGA
GTCCGCCACACCAGAGAGCTCCGGCGGCTCCTCCGGAGGATCC_TCTGAGGTGGA
GTTTTCCCACGAGTACTGGATGAGACATGCCCTGACCCTGGCCAAGAGGGCACGCGA
TGAGAGGGAGGTGCCTGTGGGAGCCGTGCTGGTGCTGAACAATAGAGTGATCGGCGA
GGGCTGGAACAGAGCCATCGGCCTGCACGACCCAACAGCCCATGCCGAAATTATGGC
CCTGAGACAGGGCGGCCTGGTCATGCAGAACTACAGACTGATTGACGCCACCCTGTA
CGTGACATTCGAGCCTTGCGTGATGTGCGCCGGCGCCATGATCCACTCTAGGATCGG
CCGCGTGGTGTTTGGCGTGAGGAACGCAAAAACCGGCGCCGCAGGCTCCCTGATGGA
CGTGCTGCACTACCCCGGCATGAATCACCGCGTCGAAATTACCGAGGGAATCCTGGC
AGATGAATGTGCCGCCCTGCTGTGCTATTTCTTTCGGATGCCTAGACAGGTGTTCAATG
CTCAGAAGAAGGCCCAGAGCTCCACCGAC_TCCGGAGGATCTAGCGGAGGCTCCTC
TGGCTCTGAGACACCTGGCACAAGCGAGAGCGCAACACCTGAAAGCAGCGGGG
GCAGCAGCGGGGGGTCA_GACAAGAAGTACAGCATCGGCCTGGCCATCGGCACC
AACTCTGTGGGCTGGGCCGTGATCACCGACGAGTACAAGGTGCCCAGCAAGAAA
TTCAAGGTGCTGGGCAACACCGACCGGCACAGCATCAAGAAGAACCTGATCGGA
GCCCTGCTGTTCGACAGCGGCGAAACAGCCGAGGCCACCCGGCTGAAGAGAACC
GCCAGAAGAAGATACACCAGACGGAAGAACCGGATCTGCTATCTGCAAGAGATC
TTCAGCAACGAGATGGCCAAGGTGGACGACAGCTTCTTCCACAGACTGGAAGAG
TCCTTCCTGGTGGAAGAGGATAAGAAGCACGAGCGGCACCCCATCTTCGGCAAC
ATCGTGGACGAGGTGGCCTACCACGAGAAGTACCCCACCATCTACCACCTGAGA
AAGAAACTGGTGGACAGCACCGACAAGGCCGACCTGCGGCTGATCTATCTGGCC
CTGGCCCACATGATCAAGTTCCGGGGCCACTTCCTGATCGAGGGCGACCTGAACC
CCGACAACAGCGACGTGGACAAGCTGTTCATCCAGCTGGTGCAGACCTACAACC
AGCTGTTCGAGGAAAACCCCATCAACGCCAGCGGCGTGGACGCCAAGGCCATCC
TGTCTGCCAGACTGAGCAAGAGCAGACGGCTGGAAAATCTGATCGCCCAGCTGC
CCGGCGAGAAGAAGAATGGCCTGTTCGGAAACCTGATTGCCCTGAGCCTGGGCC
TGACCCCCAACTTCAAGAGCAACTTCGACCTGGCCGAGGATGCCAAACTGCAGC
TGAGCAAGGACACCTACGACGACGACCTGGACAACCTGCTGGCCCAGATCGGCG
ACCAGTACGCCGACCTGTTTCTGGCCGCCAAGAACCTGTCCGACGCCATCCTGCT
GAGCGACATCCTGAGAGTGAACACCGAGATCACCAAGGCCCCCCTGAGCGCCTC
TATGATCAAGAGATACGACGAGCACCACCAGGACCTGACCCTGCTGAAAGCTCT
CGTGCGGCAGCAGCTGCCTGAGAAGTACAAAGAGATTTTCTTCGACCAGAGCAA
GAACGGCTACGCCGGCTACATTGACGGCGGAGCCAGCCAGGAAGAGTTCTACAA
GTTCATCAAGCCCATCCTGGAAAAGATGGACGGCACCGAGGAACTGCTCGTGAA
GCTGAACAGAGAGGACCTGCTGCGGAAGCAGCGGACCTTCGACAACGGCAGCAT
CCCCCACCAGATCCACCTGGGAGAGCTGCACGCCATTCTGCGGCGGCAGGAAGA
TTTTTACCCATTCCTGAAGGACAACCGGGAAAAGATCGAGAAGATCCTGACCTTC
CGCATCCCCTACTACGTGGGCCCTCTGGCCAGGGGAAACAGCAGATTCGCCTGG
ATGACCAGAAAGAGCGAGGAAACCATCACCCCCTGGAACTTCGAGGAAGTGGTG
GACAAGGGCGCTTCCGCCCAGAGCTTCATCGAGCGGATGACCAACTTCGATAAG
AACCTGCCCAACGAGAAGGTGCTGCCCAAGCACAGCCTGCTGTACGAGTACTTC
ACCGTGTATAACGAGCTGACCAAAGTGAAATACGTGACCGAGGGAATGAGAAAG
CCCGCCTTCCTGAGCGGCGAGCAGAAAAAGGCCATCGTGGACCTGCTGTTCAAG
ACCAACCGGAAAGTGACCGTGAAGCAGCTGAAAGAGGACTACTTCAAGAAAATC
GAGTGCTTCGACTCCGTGGAAATCTCCGGCGTGGAAGATCGGTTCAACGCCTCCC
TGGGCACATACCACGATCTGCTGAAAATTATCAAGGACAAGGACTTCCTGGACA
ATGAGGAAAACGAGGACATTCTGGAAGATATCGTGCTGACCCTGACACTGTTTG
AGGACAGAGAGATGATCGAGGAACGGCTGAAAACCTATGCCCACCTGTTCGACG
ACAAAGTGATGAAGCAGCTGAAGCGGCGGAGATACACCGGCTGGGGCAGGCTG
AGCCGGAAGCTGATCAACGGCATCCGGGACAAGCAGTCCGGCAAGACAATCCTG
GATTTCCTGAAGTCCGACGGCTTCGCCAACAGAAACTTCATGCAGCTGATCCACG
ACGACAGCCTGACCTTTAAAGAGGACATCCAGAAAGCCCAGGTGTCCGGCCAGG
GCGATAGCCTGCACGAGCACATTGCCAATCTGGCCGGCAGCCCCGCCATTAAGA
AGGGCATCCTGCAGACAGTGAAGGTGGTGGACGAGCTCGTGAAAGTGATGGGCC
GGCACAAGCCCGAGAACATCGTGATCGAAATGGCCAGAGAGAACCAGACCACC
CAGAAGGGACAGAAGAACAGCCGCGAGAGAATGAAGCGGATCGAAGAGGGCAT
CAAAGAGCTGGGCAGCCAGATCCTGAAAGAACACCCCGTGGAAAACACCCAGCT
GCAGAACGAGAAGCTGTACCTGTACTACCTGCAGAATGGGCGGGATATGTACGT
GGACCAGGAACTGGACATCAACCGGCTGTCCGACTACGATGTGGACCATATCGT
GCCTCAGAGCTTTCTGAAGGACGACTCCATCGACAACAAGGTGCTGACCAGAAG
CGACAAGAACCGGGGCAAGAGCGACAACGTGCCCTCCGAAGAGGTCGTGAAGA
AGATGAAGAACTACTGGCGGCAGCTGCTGAACGCCAAGCTGATTACCCAGAGAA
AGTTCGACAATCTGACCAAGGCCGAGAGAGGCGGCCTGAGCGAACTGGATAAGG
CCGGCTTCATCAAGAGACAGCTGGTGGAAACCCGGCAGATCACAAAGCACGTGG
CACAGATCCTGGACTCCCGGATGAACACTAAGTACGACGAGAATGACAAGCTGA
TCCGGGAAGTGAAAGTGATCACCCTGAAGTCCAAGCTGGTGTCCGATTTCCGGA
AGGATTTCCAGTTTTACAAAGTGCGCGAGATCAACAACTACCACCACGCCCACG
ACGCCTACCTGAACGCCGTCGTGGGAACCGCCCTGATCAAAAAGTACCCTAAGC
TGGAAAGCGAGTTCGTGTACGGCGACTACAAGGTGTACGACGTGCGGAAGATGA
TCGCCAAGAGCGAGCAGGAAATCGGCAAGGCTACCGCCAAGTACTTCTTCTACA
GCAACATCATGAACTTTTTCAAGACCGAGATTACCCTGGCCAACGGCGAGATCCG
GAAGCGGCCTCTGATCGAGACAAACGGCGAAACCGGGGAGATCGTGTGGGATAA
GGGCCGGGATTTTGCCACCGTGCGGAAAGTGCTGAGCATGCCCCAAGTGAATAT
CGTGAAAAAGACCGAGGTGCAGACAGGCGGCTTCAGCAAAGAGTCTATCCTGCC
CAAGAGGAACAGCGATAAGCTGATCGCCAGAAAGAAGGACTGGGACCCTAAGA
AGTACGGCGGCTTCGACAGCCCCACCGTGGCCTATTCTGTGCTGGTGGTGGCCAA
AGTGGAAAAGGGCAAGTCCAAGAAACTGAAGAGTGTGAAAGAGCTGCTGGGGA
TCACCATCATGGAAAGAAGCAGCTTCGAGAAGAATCCCATCGACTTTCTGGAAG
CCAAGGGCTACAAAGAAGTGAAAAAGGACCTGATCATCAAGCTGCCTAAGTACT
CCCTGTTCGAGCTGGAAAACGGCCGGAAGAGAATGCTGGCCTCTGCCGGCGAAC
TGCAGAAGGGAAACGAACTGGCCCTGCCCTCCAAATATGTGAACTTCCTGTACCT
GGCCAGCCACTATGAGAAGCTGAAGGGCTCCCCCGAGGATAATGAGCAGAAACA
GCTGTTTGTGGAACAGCACAAGCACTACCTGGACGAGATCATCGAGCAGATCAG
CGAGTTCTCCAAGAGAGTGATCCTGGCCGACGCTAATCTGGACAAAGTGCTGTCC
GCCTACAACAAGCACCGGGATAAGCCCATCAGAGAGCAGGCCGAGAATATCATC
CACCTGTTTACCCTGACCAATCTGGGAGCCCCTGCCGCCTTCAAGTACTTTGACA
CCACCATCGACCGGAAGAGGTACACCAGCACCAAAGAGGTGCTGGACGCCACCC
TGATCCACCAGAGCATCACCGGCCTGTACGAGACACGGATCGACCTGTCTCAGCT
GGGAGGTGAC_TCTGGCGGCTCA_AAAAGAACCGCCGACGGCAGCGAATTCGA
GCCCAAGAAGAAGAGGAAAGTC_[optionalP2A-GFP]_TAA

### Sequences 5. APOBEC Homologs Used for Ancestral Reconstruction.

>A7T1Z8_NEMVE/51-294
(SEQ ID NO: 220)
LGKEFCGAFYHPRSKSKQSCAIAKRGHDATLTRRYTNSKKHAEEFFLMDIDCQARHF
WNKKWQITMYLTMQPCHSTDTGGTKEDQSCCEVMIKAKEKLGNVEIVIKPTHLCQV
GKGKPEKGVRKLKTTGIELECMKEGDWKYYAQPEENPYDTSKTEELHNQQLEQ
>D8M4Y9_BLAHO/46-382
(SEQ ID NO: 221)
CMATGTKCVGAKAQSPEGFVVNDCHAEVLCRRSFIHYLIKEIQKVFEPCEGDFHLQV
KRDYHFYMYISQSPCGYGSEYETNGKAMEQSCSDKLCLLISPIYLTGIVICWDEARM
KNALADRIEVSAIVWSFFKEVVQYKEIKRERYEQAKESLMK
>I1F5T9_AMPQE/52-472
(SEQ ID NO: 222)
SLGTGSKCIGEHKMSLEGWLVNDSHAEIIARRGFVKYLIGQLKSIFERSPIKGQYSIKN
GGDASIFLSTKHSSCSDKMMRLIDKPVYLHSITVELYNQEAMERALVGRLCLSKELL
GSIVWSDFKELVQNDLPVYYDWKQSSYQKARTLFN
>E9GMF4_DAPPU/61-428
(SEQ ID NO: 223)
SMGTGSKCVGQNKLSKDGDILNDSHAEVVARRGFLRYMYHQMEILFTVDAISKKFL
QREGVSFIFFSSHTPCGDASIIVKENSMCNSCSDKLAKFLSKPIYVSHVIICPYSQSVME
RALINRFEHSANQLSSISWQKFVSLLSDNLPIYNKAKRLCYFEMWEQTRA
>K7J168_NASVI/49-389
(SEQ ID NO: 224)
SLCTGTKCLSGVELRSTGNKLSDSHAEILARRAFLRYLYHQIELIFYLEDNGIKLRDVS
FHFFSSQTPCGDCSIIKLTGEDLSCSDKLAKLMIPTIKFESIVICPYSLESMQRGIFQRFD
PSSIVWKTFLTIYDPQHPKYYHCKQYSYQNLWREKS
>F4X2X4_ACREC/49-389
(SEQ ID NO: 225)
ALATGTKCLGDSELSKGGSRLSDSHAEVLTRRAFIRYLYDQIDLIFSRNDKNKIELNS
NISFHFFSSQTPCGDCSIFKLETNSCSDKIAKLLIPPIKLESITISPFSLDAMERGLYKREN
NISIIWQSFLQIFDIRHPKYYDWKQWSYQNEWKQLKH
>E9IK04_SOLIN/50-395
(SEQ ID NO: 226)
ALATGTKCLGDSELSESGSRLSDSHAEVLTRRAFIRYLYDQIDLIFSRNDDNEIELNNN
ISFHFFSSQTPCGDCSIFKEETTSDSCSDKMAKLMIPPIKLESITICPFSLDAMERGLYKR
FNNISIVWRSFLQIFDIRHPKYYDWKQWSYQNEWKRLRR
>E2AUE8_CAMFO/49-391
(SEQ ID NO: 227)
ALATGTKCLGESELINDGSRLSDSHAEVLARRAFIRYLYDQIDLVFSWSHKNKIELNS
NVSFHFFSSQTPCGDCSIFKHEIYSCSDKMAKLLIPPIKLKSITICPFSLDAMERGLYKR
FNNTSIIWQTFLQIFDIRHPKYYDWKQWSYQSKWKQLKL
>E2B4R0_HARSA/49-389
(SEQ ID NO: 228)
ALATGTKCLGESELTNSGSRLSDSHAEVLTRRAFIRYLYDQIDLVFKRNDKNKIELNS
AISFHFFSSQTPCGDCSIFKEKTSNSCSDKIAKLMIPSIRLESITICPFSFDAMERGLYKR
FNNSSIIWQTFLQVLDIKHPKYYDWKQWSYQNKWRQLQF
>B0WPX1_CULQU/53-394
(SEQ ID NO: 229)
SLGTGTKCLGADQLSEKGDILNDSHAEVMARRGFLRFVMQQMTSIFEFNSESRKFSC
KSGISFHFFTTHSPCGDASIYETSRSSCSDKMARLLERPIYLESVVITDFCKEAIERALW
KRWDPDVVEGGGIVWATFAAVWKGADMDYVDVKNRSYREQWDALRV
>B4NPW3_DROWI/50-393
(SEQ ID NO: 230)
SLGCGTKCIGQDKQCPKGYILNDSHAEIIARRAFLRYLYHELNKIFEWDHELVCYKLK
KHYEYHFLCTHTPCGDACIGQLDTEVSCSDKLARLIHEPIYFTSFNFSEANVADIERAI
FKRWQHKEFRNGLSWLCFLELLRIRKSLYGTCKQLAYQEAWQQLKS
>B4JCX0_DROGR/56-405
(SEQ ID NO: 231)
SLATGTRCIGSSKLCARGFILNDCHAEVLARRGFLRYLNNEIMKIFSWQTAERCFTLN
ENLVFHFLSTQTPCGDACILESNIDTLSCSDKLSRLLNKPIYFTSFNFADAHLKSLQRA
LYQRWNGRSCKNSLIWLNFLNLLNLRQKLYAQCKALAYQLVWQQLKC
>B4M8L0_DROVI/67-417
(SEQ ID NO: 232)
ALATGTRCIGASKLCAKGFVINDSHAEVLARRAFLRYLHNQLLKIFSWKSAAGCFTL
NEQLEYHELSTQTPCGDACIVDSEILSCSDKLSRLIDKPIYESSLNFAEARLESLQRAV
YQRWRGRNCSTSLIWLNFLDLLHLREKFYAECKALAYQLAWRQLKC
>B4KIX6_DROMO/54-403
(SEQ ID NO: 233)
ALATGTRCIGASKLCNRGYILNDCHAEVLARRAFLRYLQNELLNVFIWKPSTGCFAL
NEQLEYHELSTQTPCGDACIVDDACGVSCSDKLARVIDKPIYFISYNFTEANPESLERA
IYRRWQGRECNSSLIWINFMDLLSVREKLYSELKALSYQLAWQQLKS
>Q29K71_DROPS/55-392
(SEQ ID NO: 234)
ALGCGTKCIGYTRHCPKGFILNDSHAEVLARRAFLRYLYHELEHIFQWAAKRGSFDL
SAHVEYHFFSTQTPCGDACIVESVVVESCSDKLARLLSKPIYFSTLNFTEARQESVER
AIYKRWQQGRDSKNGLVWLKFLNILHLREKFYASCKDLCYQQAWCQLKR
>B3MN31_DROAN/55-389
(SEQ ID NO: 235)
ALGLGTKCIPHTKLCENGFILNDSHAEVLARRAFQRYLYHELGQIFHWNCDSQCYDL
DDHVEYHELSTQTPCGDACIVEEDRSCSDKLSRLISKPIYFSTLNFADAHFESLNRAIY
KRWEGRNFSNGLVWLTFLDCISLSEKFYATCKSWSYQEAWLQLKD
>B4HX96_DROSE/54-382
(SEQ ID NO: 236)
SLGCGTKCIGESKLCPKGLILNDSHAEVLARRGFLRFLYQELKQIFHWNSELSTFDMD
EHVEFHELSTQTPCGDACILEEQVSCSDKIARLISKPIYFSSLNFDDAQLESLERAIFKR
FECRSFKNGLIWLTFLELVKLSEKFYASCKNLAYQFAWREIKE
>H2YAA2_CIOSA/58-486
(SEQ ID NO: 237)
AIGSGTTCLGESQMRPDGLILHDSHAEVIARRSFIRYLYHEIKSIFNKLIDGKCCLKPGI
SFHFYTSNTPCGDATIFVTVNQPVNTSCSDKILRLVSTPVYMRSIVVADIQALRRAFY
GRMDLNSLDFSALSWRMFKEVMECELKSYRNCKLLAYFNTWSDVKD
>C3Y8L3_BRAFL/55-488
(SEQ ID NO: 238)
AMGTGSKCIGRSKMRLEGDVLHDSHAEIIARRAFLRYLYHQLQMVFTTPGADCRCG
LKPGVKFHLFTSHTPCGDASIFKGSKSTIDQSCSDKLARFMTEPVYFSTIVVCPYSSYA
MYRGIVDRCAGIQDLAAIMWETFKQLLNERRPAYGDYKRAAHHTAKTCFLR
>F1R076_DANRE/54-459
(SEQ ID NO: 239)
SLGTGTKCIGRSAMSMKGDVLNDSHAEVIARRGSVRYLTEQLLKVFCAGSEKGKWR
LKAGVSFLFFTSQTPCGDASIFMSGSEPCETSCSDKLARYLQEALYFSAVLVSPYSHP
ALRRALHTRCSHVKDLAAISWHSFLKVVAAELPEYWDYKQAAYQLAWTQLRL
>H2S4J7_TAKRU/61-472
(SEQ ID NO: 240)
SLATGTKCIGRTALSPNGDVLNDSHAEVIARRGCVRYLIQELHRVFCRAEQQGKWKL
KPGVSFHFFTSHTPCGDASIIMSDSQPCPGSCSDKMARYLQEALYFTSVVVCPYSQEV
MHRALVARCLHVSDLAAISWHSFLSLVSSSLPSYWDYKRASYQQAWQQLHS
>ADAT1_XENTR/59-465
(SEQ ID NO: 241)
AMGTGTKCIGQAKLRKTGDVLQDSHAEIIAKRSFQRYLLHQLSLLFIPGTEKGKWML
RPEISFVFFTSHTPCGDASIIVISHELGHGSCSDKMARFLQQPIYLSAVVVCPFSQDAM
ERALYNRCHKVLSLAAVSWNTFRELVQKQRSEYWDYKAAAYQEAWNCLRQ
>U3JIM9_FICAL/63-494
(SEQ ID NO: 242)
AMGTGTKCIGRNKMRKTGDILNDSHAEVVAKRSFQRYLLHQLWLIFSPGTETGKWK
LKPNIIFIFFSSHTPCGDASIFISEPELSKNSCSDKLARFLQYPVYLSAVIVCPYSQEAMR
RAVIERCQHVSSLAAISWHKFQRLMTEDLPDYWDYKQAAYQEAWRVLRS
>ADAT1_CHICK/63-496
(SEQ ID NO: 243)
ALGTGTKCIGLNKMRKTGDVLNDSHAEVVAKRSFQRYLLHQMRLIFIPGTETGKWK
LKPNIIFIFFCSHTPCGDASIIIRETELSKSSCSDKLARFLQYPVYLSAVIVCPYSQEAMQ
RAVIERCRHISLLAAISWHEFQKLVTENLPDYWDYKEAAYQEAWKALRS
>U3IY34_ANAPL/63-495
(SEQ ID NO: 244)
AMGTGTKCIGQNKMRKTGDILNDSHAETVAKRSFQRYLLHQMCLIFIPGTETGKWK
LKPNIIFVFFSSHTPCGDASIIISEPELSKNSCSDKLARFLQYPVYLSAIIVCPYSQEAMR
RAIIERCQHVSFLAAISWHAFQKLVTENLPDYWDYKEAAYQEAWKVLRS
>F7EEN0_ORNAN/78-517
(SEQ ID NO: 245)
AMGTGTKCIGQSKMRKDGDILNDSHAEVIAKRSFQRYLLHQIEAIFIPGTEVGKWKL
KPDLSFVFFSSHTPCGDASIIMLDVEPCPRSCSDKLARLLQDPVYLAAVVICPYSPEAV
RRAVIDRCRHVSPLAAISWGAFQKLVGDKQPAYWDYKEAAYQETWKALQR
>F7G3V9_MONDO/64-497
(SEQ ID NO: 246)
SMGTGTKCIGLSKMRKNGDILNDSHAEVIAKRSFQRYLLHQLRSIFMPGTKTGMWK
LKPDLHFVFFSSHTPCGDASIIMLELEPCSHSCSDKLARFLEEPVYLAALVICPYSPEA
MKRAVIDRCQHVSSLAAISWNLFQNLVSSKRPEYWEYKEAAYQEAWKTLQK
>G1T9N5_RABIT/63-492
(SEQ ID NO: 247)
SMGTGTKCIGQSKMRKSGDILNDSHAEIIAKRSFQRYLLYQLHMIFVPGTQRGLWKL
RPDLSFVFFSSHTPCGDASIIMLEFEPCCSSCSDKVARFLEEPIYLSAVVICPYSQEAMQ
RALLGRCQNVSALAAISWRSFQKLLSDKWPDYQEHKEAAYQEAWRTLRK
>G1Q6Y1_MYOLU/63-485
(SEQ ID NO: 248)
SMGTGTKCIGQSKMRKSGEVLNDSHAEVIARRSFQRYLLHQPHLIFIPGTQRGLWKL
RPDVFLFVSSHTPCGDASIIMLEFEPCYSSCSDKLARFLEEPVYLSAVVICPYSQEAMQ
RAPVRRCRMSPLYAAISWRSFQELLSDKWPDYQEYEEASYQEAWSALRK
>F6Z5W8_HORSE/63-490
(SEQ ID NO: 249)
SMGTGTKCIGQSKMRKSGDILNDSHAEIIARRSFQRYLLHQLHLIFVPGTRRGLWKLR
PDLLFVFFSSHTPCGDASIIMLEFEPCCSSCSDKLARFLEEPIYLSAVVICPYSQEAMQR
ALTGRCHNISALAAISWRSFQKLLSDKWPDYQEYKEAAYQEAWSALRK
>M3YEQ1_MUSPF/71-497
(SEQ ID NO: 250)
SMGTGTKCIGQSKMRKSGDILNDSHAEVIARRSFQRYLLHQLHLIFVPGTQRGLWKL
RPDLSFVFFSSHTPCGDASIIMLEFEPCCSSCSDKLARLLEEPVYLSAVVICPYSQEAM
HRALTGRCQNVSALAAISWRSFQKLLSDKWPDYHEYKEAAYQEAWSALRK
>ADAT1_MOUSE/63-492
(SEQ ID NO: 251)
SMGTGTKCIGQSKMRESGDILNDSHAEIIARRSFQRYLLHQLHLIFVPGTQRGLWRLR
PDLSFVFFSSHTPCGDASIIMLEFEPCCSSCSDKMARFLEKPIYLSAVVICPYSQEAMR
RALTGRCEETLVLAAISWRSFQKLLSDEQPDYQEYKDAAYQEAWGALRR
>H0VKI8_CAVPO/62-489
(SEQ ID NO: 252)
SMGTGTKCIGQSKMRKSGDVLNDSHAEVIARRSFQRYLCHQLQLIFVLGTQKGQWK
LRPGISFVFFSSHTPCGDASIIMLGFEPCYSSCSDKVARFLEEPIYLSAVVICPYSQEAM
HRALIGRCQNISALAAISWRSFQKLLSDMWPNYQEYKQAAYQEAWSALRK
>G7Q1N8_MACFA/63-495
(SEQ ID NO: 253)
SMGTGTKCIGQSKMRKSGDILNDSHAEVIARRSFQRYLLHQLQLIFVPGTQKGLWKL
RRDLFFVFFSSHTPCGDASIIMLEFEPCCSSCSDKMARFLEEPIYLSAVVICPYSQEAM
QRALTGRCQNVSALAAISWRSFQKLLSDKWPDYQDYKEAAYQEAWSTLRK
>13MW85_ICTTR/63-492
(SEQ ID NO: 254)
SMGTGTKCIGQSKMRKSGDILNDSHAEVIARRSFQRYLLYQLQLIFVPGTQRGLWKL
RPNLLFVFFSSHTPCGDASIIMLEFEPCCGSCSDKMARFLEEPIYLSAVVICPYSQEVM
QRALIGRCQNVLALSAISWRSFQKLLSDKWPDYQEHKEAAYQEAWSALRK
>D8SUQ7_SELML/51-383
(SEQ ID NO: 255)
ALGTGTKCLGGSQRSLAGDTINDCHAEVIARRTLLKLLYTDIGSNGRLRMRPEIRLHL
YISQSPCGDACGVDSCSDKIARFLNEPVYISTITVSDLACIEALQRATFGRLCSVARNY
SISWRSFRHLLTHFFPGYLECKKLAYLEAKEILLH
>M0SLB0_MUSAM/69-408
(SEQ ID NO: 256)
ALGTGTKCIGGSLLSPTGDVVNDSHAEIIARRSLLRYFYAEIERAFDTSCCGQTKYRM
NPGWGLHLYITQLPCGVFSYPTLRELSCFNKITRILQPVYLSTLTVSVKTNYLEKAIYD
CMERLHIKYSICWEVFVSLQSRLSLQYHGLKAMAYQSSLKMLR
>M1BYG7_SOLTU/64-418
(SEQ ID NO: 257)
SLGTGTKCIGRSRRSSKGDVVNDSHAEIIARRALLRYLYSEIQDMFESDGLGTKKLK
MKHGWQLHLYISQLPCGVASPGSELAHDSMSCSDKIARFLEPIYIFSVTISIIEDEVMR
AIHERVLPLSNKYSICWESFLSLSHNCVGEYRELKDKSYNLASETFK
>K7KC56_SOYBN/58-388
(SEQ ID NO: 258)
AMGTGTKCLGRSLLRTCGDVVHDSHAEVIARRALIRFFYTQIQPFDPGCLNKGKYSL
KKDWKLHMYISQLPCGDASLSVSPLSCSDKIARFLQPVYLSSITVFRLEDSLKRALYE
RMLPLSNEYSICWEVFLSLGPENLIGYRELKDGAYHLASKIFK
>F4HU58_ARATH/50-411
(SEQ ID NO: 259)
ALGTGTKCVSGSLLSPRGDIVNDSHAEVVARRALIRFFYSEIQRSIDSSCPGEVKYKLK
SGCLLHLYISQLPCGYASTSSPLYKKISCSDKIARVLQPVYISTITVFSLADHLRRSLYE
RILPLSDEYSLCWELFLKETHGHKREYRELKNKAYYLMSKIFK
>W4XXD3_STRPU/717-1055
(SEQ ID NO: 260)
SLGTGNRCVTGDKLSMEGRTVNDSHAEIITRRAFLRYLYNQLQAILTQGTNGKLRLL
PDVSLHLYISTAPCGDGAQFSRTDENESSCSDKVASFIEPMYLSSISLSLYHHGHLARA
VCCRVSSELDNLLSINWSEYQQTCRLFDRTYHDAKLSAYYTAKQYLKC
>11GJ01_AMPQE/629-965
(SEQ ID NO: 261)
SFGTGTRTASGDLLDLKGEVVFDSHAEIIARRGLKMFLYQELQVIFEANEDGKLRVK
KSIKFHLYISTAPCGDGAQFSRLDNRLSSCSDKIGSFVEPVYLFSLSLSLHHHGHLSRA
VCCRFHELGPYLSMNWNEFLSLSSVSGHTYAEAKSLAYQEAKGLLHK
>A7REZ9_NEMVE/161-491
(SEQ ID NO: 262)
SLGAGNRCVTGQRLSMEGKVVNDSHAEIIARRSLLRFFYAQLHAIFEKNNSRRLAVR
QGVSFHLYISTAPCGDGALFTPRENTDLSCSDKICRFIEPVYLESLTLYLYDHGHLAR
AVCCRLQDLGAELSVNWESFKELCQRTDREYSQAKQMAFQNAKRELFE
>K1PWV0_CRAGI/737-1070
(SEQ ID NO: 263)
SIGTGNRCITGPQLSLEGNTVNDSHAEIITRRGFIRFMYKQLQALFEPSPSGKLRLKDNI
TFHLYISTAPCGDGALFSPRDSNNASCTDKLCRLMDPVYLDSLTLLLYDHGHLARAM
CCRLARDINSLLSVNWDNFKQVCADLGKQYGKAKTAAFQTAKKALIK
>T1J5P4_STRMM/754-1087
(SEQ ID NO: 264)
SMGTGNRCISGERLSQEGLVVNDSHAEIVTRRGFLRFLYKQLIEIFEPSENRKFRVKPD
VTFHLYISTAPCGDGALFSKTDVSELSCSDKIARFVDPIYLTSITLYLYDHGHLSRAVC
CRLSELDTLHSLSWASFKEACIKHQRDYYLVKTAAFQKAKKVLLE
>M4A7Z3_XIPMA/994-1320
(SEQ ID NO: 265)
SLGTGNRCVKGEELSLKGETVNDCHAEIISRRGFIRFLYSELLKIFEPAEENKLKIKPDI
TFHLYISTAPCGDGALFDCSEKSCSDKILRFLHPIYLKSITLYLYSHGHLTRAVCCRLA
RAFSQNSSVNWCLFRSLCQRCGRTYAQAKTSAFQLAKQQFFE
>M3XGS0_LATCH/812-1140
(SEQ ID NO: 266)
SIGTGNRCVKGEELSLKGETVNDCHAEIISRRGFIRFLYSELMKFLEVVSDGRMKIKT
GVTFHLYISTAPCGDGALFDCSEKSCSDKILRFLHPVYLSSVTLYLYSQGHLARAICC
RMSRAFQEGSSVNWQLFQQICTKTDRKYQEAKEAAFQKAKVHFVQ
>U3IQ81_ANAPL/729-1061
(SEQ ID NO: 267)
SIGTGNRCVKGEELSLKGETVNDCHAEIISRRGFVRFLYSELMKIFEPAGKKRLKIKSN
VTFHLYVSTAPCGDGALFDCSDKSCSDKILRFIEPVYLSSVTLYLYSQGHLTRAICCR
MVRVLQKRSSVNWALFQQLCAKNKRKYSEAKEAAYQEAKQRFFS
>F6ZMB0_XENTR/760-1089
(SEQ ID NO: 268)
SIGTGNRCVKGEELSLRGETVNDCHAEIVSRRGFISNTIXSQLMKIFEEAEGDLLRVRP
GVTFHLYISTAPCGDGALFDCSDKSCSDKILRFMEPVYLSSLTLYLFSQGHLTRAICCR
MSRAFQNQSSVNWTLFQQLSVLRGRHYSDVKATAYQTAKGQLFR
>F7GMY9_MACMU/591-920
(SEQ ID NO: 269)
SLGTGNRCVKGDSLSLKGETVNDCHAEIISRRGFIRFLYSELMKIFEPAKGGEKLQIKK
TVSFHLYISTAPCGDGALFDCSDKSCSDKILRFLQPIYLKSVTLYLFSQGHLTRAICCR
VTRAFEDGTSVNWLLFKKLCSFRYRRYGEAKKAAYETAKNYFKK
>C3YCU6_BRAFL/320-659
(SEQ ID NO: 270)
AMGTGNSCVMGKNIGTDGRTLNDCHAEVVVRRSLLRYLYRELKNIFEKHSDSLLVL
KEGVSFHLYLTTAPCGDAATHITRDTRVQSCSDKLAKFIEPVYLSSVTFTNYDHGQLS
RALCCRIDDHISADISLNWMRFKAMCTLARRQYNQAKMMCYQEAKRQLYD
>H3ABU2_LATCH/179-499
(SEQ ID NO: 271)
ALGTGNSCYAGWLAFDGRLLHDCHALVVVRRTLQRFLYKQLLLIFCSSPGGSLSLRP
GIFFHLYLGKVPEGAARTTFMYAPRNPSESDKMTRFIQPIYITSVVLHFYDRNTVSQVI
NKRLEGALIRKSSLNWSSFRKVAEKTGRSYHSVKLRAYQKVKKQVNC
>H9G4E7_ANOCA/55-372
(SEQ ID NO: 272)
ALGTGDICYEGWMEFNGRRVHDMHGMVVARRVLLRYFYKQLLMIFCPAGDGILAL
KPKYFLHLYLSRTPSGASENFHLAPVVQSGSDKLTRIILPVYITSVVLPYQDHTVLHE
VVNDRVQLGPGNGLSLNWSMFRKVVQEMKREYHKGKVQAYQSAKLQMYT
>GINZA7_MYOLU/269-591
(SEQ ID NO: 273)
ALGTGSSSCAGWLEFSGRQLHDCHGLVVARRALLRFLYRQLLLVLTPQPGPGFVLKP
RIFIHFYISNTPKGAAHDIYPPAASASDKLSRFLPPLYATSLVLPCHDPPTLSSAIHTRL
DRVSGSCLSLNWRAFRQAAQALGKSYEEAKAYQEARQQLSL
>F1M8B2_RAT/237-552
(SEQ ID NO: 274)
ALGTGSSSCAGWLEFSGRRLHDCHGLVIARRALLRFFFRQLLLVLTPQPGSGFALKPG
VFLHLYVSNTPKGAAHDIYLASPSASDKLARFLPPLYATSLVLPCHDPPTLNRAIHSR
LDSVLGSCLSLNWAAFRQVARALEKPYEAAKAYREARQQLSL
>H0X0C9_OTOGA/275-589
(SEQ ID NO: 275)
ALGTGNSCCAGWLEFSGRQLHDCHALVVARRALLRFLYRQLLLVLAPQPGPGFALK
PRIFLHLYVSNTPKGAAHDIYSASTSASDKLARFLPPLYSTSLVLPCHDPSTLSRAIHSR
LDGALGPVLSLNWRAFRQAAGALGKPYEAAKAYQEARRRLSL
>F6QYL4_MACMU/258-573
(SEQ ID NO: 276)
ALGTGSSCCAGWLEFSGQQLHDCHGLVIARRALLRFLFRQLLLVLAPQPGPGFTLKP
RVFLHLYISNTPKGAARDIYPTSPSASDKLARLVSPLYSTSLILSCHDPPTLSRAIHTRL
DSVLGPCLSLNWRAFHQVARAVGKPYEAAKAYQEARRQLSL
>H3A635_LATCH/258-579
(SEQ ID NO: 277)
ALGTGDFNYSQCICRDGRVVHDSHGVVMARRSLLRFLYRQLLLIFCIEPTSKLTIKPN
ANIHLYLNQLPKGAAQIKSQLRPQSQSASDKLTRFIQPVYISSVLIANCTDTRGLEIAV
KQRVDDALTSRLSLNWSRFNLLAKESNREYHDAKIMSYQEAQCLLKS
>H2PE88_PONAB/193-512
(SEQ ID NO: 278)
AIGTGEYNYSQDIKPNGRVLHDTHAVVTARRSLLRYFYRQLLLIFCTEPTSNLTLKQN
INICLYMNQLPKGSAQIKSQLRPHSESSSDKLTRFIQPVYISSILVGNCSDTRGLEIAIKQ
RVDDALTSKLSLNWSRFNLLAKEAKKYHAAKCMSYQEAKCKLKS
>H0VR27_CAVPO/289-609
(SEQ ID NO: 279)
AVGTGEYNCSQCIKPNGRVLHDTHGVVTARRSLLRYFYRQLLLIFCTEPASDLTLKQ
DINIYLYMNQLPKGSAQIKSQLRPNSESSSDKLTRFIQPVYISNILVVGNCSDTRGLEIA
IKQRVDDALTSRLSMNWSRFNLLAKEAKRDYHAAKCMSYQEAKTLLKS
>GINDQ0_MELGA/165-484
(SEQ ID NO: 280)
ALGTGDCNYSNDYSPEGRVVHDSHAIVTARRSLLRYFYRHLLLLFCTAPDSKLTLKR
NISIYLYMSQLPKGSAQIKTQFCPHSESASDKLTKFIEPVYINTILVGNCRSLKGLEIAIR
QRIDDALTSKLSLNWSRFKCLAGRAKRTYHEAKVKSYQEAKKLLHS
>U3IGD7_ANAPL/256-575
(SEQ ID NO: 281)
ALGTGQCNYSQDYQPNGRVLHDSHAIVTARRSLLRYFYRHLMLIFCTAPGSKLVLK
QNTNIFLYMNQLPKGSAQLKSQLHPQSESATDKLTKFIEPVYINSILVGNCKDTRGLEI
AVKQRIDDALTVELSLNWSRFKSLAKEAERNYHKAKIQSYQEAKKLLHS
>H0YUT0_TAEGU/165-484
(SEQ ID NO: 282)
ALGTGECNYSRRFESCGRLLHDSHAVVTARRSLLRYFYRHLLLIFCTAPGSKLTLKR
NITLSLYMNQLPKGTAQLESEVHPQSESAGDKLTKFIEPVYISNILVGSCKDTKGLDIT
IKQRLDDELTSKLSLNWSRFRMLAREAGRDYHEAKVKSYQEAKSLLQS
>W5M4T6_LEPOC/238-558
(SEQ ID NO: 283)
AIGTGDTNTNQHATANGRLLHDSHAVVTARRSLLRYLYRHLLLIFQLDLNSQLTLKR
NITIHLYMNQLPKGSAQLPPRLHPCSNSASDKITQFIEPIYVGSILIASCSDVRGLEIAV
KQRVEGITSKLSMNWSRFNLVAKEAEREYYEAKMSSYQEAKIVLKT
>F1RC56_DANRE/229-549
(SEQ ID NO: 284)
ALGTGSSNTKASPAPTGRILHDSHAVVTARRSLMRFLYRNLLLVFQQDETTKLSFKN
HITLHLYLSQLPKGASQIPSQLRPLSNSATDKVMQFIEPIYMSSIFIGSCSDIRGMEMAV
NQRVDGITSALSLNWSRFNLVAKESQREYREAKMMAYQEAKSMLKS
>W5LAE0_ASTMX/254-574
(SEQ ID NO: 285)
ALGTGNLNTKESLTPSGRILHDSHAVVTARRSLMRYLYRHLLLIFQQDQNTKLSLKS
HITLHLYLNQLPKGAAQIPSHLRPLSNSATDKITQFIEPIYVSSILIESCSDTRGMEVAIN
QRVDGITSKLSLNWSRFNLVSKEAQREYREAKMMAYQEAKSVLKS
>H2N2Y7_ORYLA/166-486
(SEQ ID NO: 286)
ALGTGGFNTRESISSDGRIVHDSHAVVTARRSLMRYLYRNLLMVYQHKSNSNLSLKT
GISLHLYVNSLPKGAAVIPSKLYPLSSSTADKITQFIEPIYVQSILISCCTDVRGMEVSV
CQRVEGVTSQLGINWHRFKLVAKEAQRHYREAKRMAYQEAKSVLRA
>G3PWS7_GASAC/162-482
(SEQ ID NO: 287)
ALGTGNFNAKESASTGGRIVHDSHALVSARRSLMRFLYRHLLMVFEQSGSGLSLKSG
ITLHLYVNQLPKGAAQIPSQLRPLSNSATDKLTQFIEPVYVHSILVVGCSDVRGLKMS
VSQRVEGITSQLGINWHRFKLVAKEARRLYREAKRMAYQEAKNVLRA
>E4WSB5_OIKDI/481-800
(SEQ ID NO: 288)
ALGTGTKTMTGDYISSIGTAVIDCHGEIISRRNLKRYFYAELQKIFERKDGQFALRDGI
KFHLYINTTTCGDARVFNPNSDFESSCSDKVMRLIEPIYLSSISLGLYHRQHFPRAMFE
RIDDIGLDFSINWQLYLLGYATKIYLDAKNLSYQSVKNGLYE
>X1WRM4_ACYPI/235-549
(SEQ ID NO: 289)
CLATGTKCLSGNYLSLSGESLHDCHAEILTRRCLLKFFYKELMEIFVFSGDKKFKLAE
DIAFHLYINTAPCGEARVFSFCDENSCSDKLTLFIEPTYLESISISVFNINHMKRTIYGR
VENSIDKYCVNWKEYIKLCSLSTLIYDLLKNKNYIAAKNQILS
>H3EEX4_PRIPA/256-594
(SEQ ID NO: 290)
SLATGNKCIKATSLSFDGCAVNDCHAEILTRRGLVRWLYTQVQLLVEQGEEGGKLR
LRKRFSLHLFISTAPCGDGRVYQFGSNKDNSCSDKVLKFLHPLLICLSIYLSSLALHFT
RESCIARACYGRVARFKPVSANWTRFAALTPYKDVKRSAYSKTQFELIR
>H2W2W9_CAEJA/169-490
(SEQ ID NO: 291)
SLSTGNKGLRGDKVVSDGTALIDCHAEILARRGLLRFLYSEILKIFMKKGTL VLRPGIS
FHLFINTAPCGAGRIDNKAKSVKASCSDKLLRFVEPIYYSSIAVEKNNVERLQRAVFG
RAASSSVNWELMVHVCAITQTYDELKAGCYETEKKAFIA
>A8WK36_CAEBR/168-488
(SEQ ID NO: 292)
SLATGNKGLRGDQITSDGSALIDCHAEILARRGLLRFLYSEVLKIFETGKLALRQGISF
HLFINTAPCGTARVDRKVRTAEASCSDKLLRFIEPVYYASIAVEQNNFVRMNKAVYT
RASSGSMNWELMVKTCFLTKTYEEMKAGCYSAAKKSFIM
>G0MG92_CAEBE/170-489
(SEQ ID NO: 293)
SLATGNKGLSGDKIRSDGSALIDCHAEILAKRGLMRFLYSEVLKIFLESGLILKLGITF
HLFINTAPCGTARVDKKMRNPESSCSDKLLRFMNPIYYSSIAVEKSNFERMNRAVND
RAAGGSMNWELMKTVCTLAKIYEELKAGSYAATKKEFYA
>E3NFH7_CAERE/177-503
(SEQ ID NO: 294)
SLSTGNKGLRGDKIVNDGSALIDCHAEILARRGLLRFLYSEVIKIFERGTLVLKKGILF
HLFINTAPCGTARVDRRMKTAEASCSDKLLRFIEPIYYTSIAVEQNNFDRINKAVFAR
AANGSMNWELMMATCKLTQTYDELKAGSYLNAKQSFIK
>ADR2_CAEEL/167-486
(SEQ ID NO: 295)
ALSTGNKGLRGDKIVNDGTALIDCHAEILARRGLLRFLYSEVLKIFTKGKLVLKPGISF
HLFINTAPCGVARIDKKLKTSDNSCSDKLLRFIDPIYYSSIAVELNNADRLRKAVYSR
AATSSMNWELMITICTLTKTYEELKAGSYAAAKKSFIT
>A7SFG1_NEMVE/255-570
(SEQ ID NO: 296)
SIGTGTKFISGEYISDKGYAVNDCHGEIIARRGLRKFLYNQLELIFELKPSGYGLKDQV
EFHLYISTSPCGDARVFSPHEPEEKSCSDKICKFIEPVYFTSIILSLYRYNHMARAMYE
RVGPVEDHSLNWTKFLALWARQYDEAKLSAYQKAKASLMM
>T111Q0_RHOPR/253-578
(SEQ ID NO: 297)
SVGTGTKCVGGEHISVKGAVLNDSHAEIISRRGLVRYFYSQLLLIFIRNEDGQFCLKPE
IRFHLYISTSPCGDARIFSPHDNTSSCSDKVAKFIEPIYLHSIVVSLFHQTHLRRALYGRI
ENTLIGHSINWIKFHELLRKTGITYSDAKELVYKDAKTYMIA
>D6X3T2_TRICA/272-590
(SEQ ID NO: 298)
SVTTGTKCISGEHISMNGCSLNDMHAEILSRRCLITYFYDQLELIFTQREDGKYKLKP
GLDFHLYINTAPCGDARIFSPHEEASSCSDKICRFIEPIYLKSIVLSLMREAHMYRALC
GRIENTIQGFAVVWKRFARLVGSVTEIYCDVKDAVYKAAKTNLYE
>E2BL35_HARSA/335-651
(SEQ ID NO: 299)
CVTTGTKCVSGEHLSVSGGAVNDCHAEVVARRCLCEYLYKQLELILEPAKKGFKLK
QGIQFHLYINTAPCGDARIFSPHEESASCSDKIARFIEPIYFHSIVLSLLNPSHMYRAVC
GRIENTIQGYSVNWRRFFNLLGTIEDVYLEAKLSVYSLAKRQLKD
>B4MGV3_DROVI/350-668
(SEQ ID NO: 300)
SVSTGTKCVSGEHMSVNGAVLNDSHAEIVSRRCLLKYLYAQLDLIFVRNTDGQYKL
KSGVHFHLYINTAPCGDARIFSPHETGASCSDKIARIIEPVYLHSIVLSLLHPEHMYRA
VCGRIEKSIQGFGINWDKYGFLMKGMQYGETKADVYQTAKQELFS
>Q17109_AEDAE/283-612
(SEQ ID NO: 301)
SLATGTKCVSGEHMSVTGSVINDSHAEIIARRGLLDFFYTQLDLIFVAPTDGTYKLKD
GIHFHLYINTAPCGDARVFSPHENMDASCSDKISRVIEPIYLHSIVLSLLHPAHMYRAI
CGRIENSIQGFSINWRRFAAVIEHARYGETKMCVYQQAKKELFA
>K1QNQ5_CRAGI/353-676
(SEQ ID NO: 302)
SVSTGTKCINGEYMSDQGLAVNDCHAEVIGRRSLMRYLYFQLGKIFQEKEGGFMLK
PNIHFHLYISTSPCGDSRIFSPHEQEASCSDKIARFIEPVYFDSLILSLYHGDHLSRAVYS
RISNIENFAVNWKRFLQLCGLTGQSYADAKVTVYQTAKQQMYL
>C3XSL3_BRAFL/39-363
(SEQ ID NO: 303)
SLATGTKCINGEYMSDQGMALNDCHAEIVSRRSLLRYLYSQLDLVFEPKEDGKYRL
KDNIQFHLYISTSPCGDARIFSPHETDASCSDKIARFVEPIYLSSIILSLYHGDHLSRAVY
QRLGELEQFSVNWQCWNKLFGTTGRHYSEAKLLNYQAAKQEMVK
>H2QL56_PANTR/370-731
(SEQ ID NO: 304)
SVSTGTKCINGEYMSDRGLALNDCHAEIISRRSLLRFLYTQLELIFQKSERGFRLKENV
QFHLYISTSPCGDARIFSPHELEAASCSDKIARFVEPIYFSSIILSLYHGDHLSRAMYQRI
SNIEDFSVNWCRWMRVHGHLLRSYHESKLAAYQAAKARLFT
>F7DBJ2_HORSE/369-690
(SEQ ID NO: 305)
SISTGTKCINGEYMSDRGLALNDCHAEIIARRSLLRFLYTQLELIFQKSERGFRLKENV
QFHLYISTSPCGDARIFSPHELEASCSDKMARFVEPIYFSSIILSLYHGDHLSRAMYQRI
SNIEDFSVNWCRWMRVHGNLLRSYHESKLAAYQAAKACLFK
>H3CU13_TETNG/420-741
(SEQ ID NO: 306)
CVSTGTKCINGEYMSDRGLALNDCHAEIIARRSLIRYLYSQLEFIFVHYEKGYRLKDN
VQFHLYISTSPCGDARIFSPHEVEASCSDKIARFMEPIYFSSIILSLYHADHLSRAMYQR
IADIEDFSVNWSRWVRLHSSILSNYHEAKQAAYHSAKQALIK
>H2N2D8_ORYLA/315-637
(SEQ ID NO: 307)
CISTGTQCIDGEHLSEDGLTVNDCHAEVVARRCLVRFLYSQLELIFRRGECGRFRLKD
KVQFHLYVSASPCGDACIFSPHDVETSCSDKMARFTEPLYFSSIIVSLYHAAHMSRAM
YGRIGPLEGFSENWCRWMQLHSSFLRVYHQAKRAAYCSAKHTLYR
>13JK85_ORENI/388-710
(SEQ ID NO: 308)
CVSTGTKCIGREYMSNRGLALNDCHAEIVARRSLIRFLYSQLEYIFMRCDNSQFRVKE
NVQFHLYISTSPCGDARIFSPHEVDASCSDKIARFTEPIYFSSIILSLYHADHLSRAMYQ
RITEIEEFSVNWSRWMNLHCSMLWIYHDTKQAAYHSAKETLFR
>H2N9L5_PONAB/404-725
(SEQ ID NO: 309)
ALSSGTKCISGEHLSDQGLVVNDCHAEVVARRAFLHFLYTQLELIFVRLKEGYRLRE
NILFHLYVSTSPCGDARLHSPYETDESCTDKIARFVEPVYLQSIVVSLHHTGHLARVM
SHRMEGVGQFSVNWARWAQLYGTRTPSYCEAKLGAYQSVKQQLFK
>F6VCE0_XENTR/367-729
(SEQ ID NO: 310)
ALSSGTKCINGEYLNDQGLVVNDCHAEIIVRRAFLHFLYTQLELIFIRLKEGYRLRENI
MFHLYVSTSPCGDARLHSPYESDESCTDKITRFIDPIYLQSIIVSLHHTGHLSRVMSHRI
EDLGNFSLNWTRWARLYGTRIGNYCDAKLAHYQSVKQQMFK
>H3C3F5_TETNG/393-715
(SEQ ID NO: 311)
ALSTGTKCINGEYLSDQGLVVNDCHAEVTARRALLRFLYSQLEFIFVRHKERYRLRD
NIHFHMYISTSPCGDGRLNSPYESDHSCTDKITRFVEPVYLHSVTISLRHTGHLGRVLN
QRLERLGPMSVNWTRWSRLYRTHASSYAEAKMAAYQSVKQQWFR
>W5LI18_ASTMX/382-706
(SEQ ID NO: 312)
ALSTGTKCINGEYMSDQGLVVNDCHAEVTTRRALLRFLYSHLELKLVQQGRGFRLR
VCVIYHLMAFVADCQTARFASPFFLSFSCTDKIARFVEPVYLSSLTVSLRHTGHLSRT
LSQRLERLGPYSVNWTRWTRLYRACAPGYCEAKQAAYQTAKEHWVR
>X2BQ53_DANRE/411-730
(SEQ ID NO: 313)
ALSSGTKCINGEYISDQGLVVNDCHAEITTRRALLRFLYSQLELIFVRHKDSLRLREN
VLFHMYISTSPCGDARVNSPYESDFSCTDKISRFVEPVYLYSLTVSLRHTGHFSRMMN
HRMEKAGPYSVNWARWVRLYRVRGASYCEAKQAAYQTVKLQWLK
>W5N363_LEPOC/428-750
(SEQ ID NO: 314)
AISTGTKCINGEYISDRGLVVNDCHAEIIARRAFVRFLYSQLELIFIRHKEAYRLRENIL
FHMYISTSPCGDARLNSPYEADESCTDKITRLVEPVYLHSLIVSLHHTGHLSRIVTHRL
DRMGHFSVNWARWAKLYRTQVPNYCEAKLAAYQTVKQQLFK
>H2LP25_ORYLA/349-671
(SEQ ID NO: 315)
SLATGTKCLDLEDESDSGRILRDCHAEVISRRALVRFLYAQLELIFVRNKDNFRLQEGI
LFHMYVSSSPCGDARLNCPYEAAFSCTDKLAKLVEPIYLHSLTVTLCHTGHLARGMA
RRLTPVKHISLNWTRWLQLQRGPVSGFSACKMSAYQRALRRFGS
>I3KMJ4_ORENI/382-706
(SEQ ID NO: 316)
SLATGTKCLDLGGVSDSGCTLTDCHAEVVSRRALVRFLYSQLELIFIPNKSCGDFRLR
EGVHFHMYVSSSPCGDARLNCPYEAAFSCTDKLAKLVEPVYLQSLTVTLSHTGHLG
RAMARRLAPIKHISVNWARWLRLQQGPVIGYCASKMSAYQRAVQQFSS
>M3ZX42_XIPMA/284-606
(SEQ ID NO: 317)
SLATGTKCLDLDRLNEDGWTLRDCHAEVLSRRALVRFFYSQLELIFVPDKDSTHFRL
QEGIRFHMYVSLSPCGDARLNCPYEPAFSCTDKIAKLVEPIYLHSLTVRLSHTGHLGR
AVTRRLARVKHVCVNWSCWLRLENTPVSGYQASKMGAYQKAMQQFSN
>G3PZQ1_GASAC/403-718
(SEQ ID NO: 318)
SLATGTKCRDSDGVGDREGTLSDCHAEVLSRRALVRFLYAQLELIFVSNTAGGFRLR
DGVFFHMYASSSPCGDARLNCPHEAAFSCTDKIAKLVEPVYLHSLTVTLSHTGHLGR
AVARRLAPVRRIKIIAARSRDVLTRHLNSYSGSKMAAYQRAMQQFTG
>H3CRD2_TETNG/277-601
(SEQ ID NO: 319)
SLATGTKCRDRGSAADGAGSLSDCHAEVISRRALLRFLYSQLELVLTPKPGGYRLRD
GLLFHMYVSCSPCGDARLNCPYEAAFSCTDKLAKLIEPVYLQSLTVTLSHTGHLSRA
LTRRLAPVRRTSSNWARWRRLQQAHLDGYAGWKAAAYQRVMQTESS
>H2S0X8_TAKRU/296-626
(SEQ ID NO: 320)
SLATGTRCPGRAGARDRAGTLSDCHAEVISRRALLRFLYWQLELIFTPNSGGCRLRD
GVLFHMFVSSSPCGDARLNCPYEAAFSCTDKLAKLIEPVYLHSLTVTLSHTGHLSRA
MARRLAPVRQISTNWVRWQQLQQPQLNGYSGWKGAAYRRMMQNFIS
>K1RR67_CRAGI/135-316
(SEQ ID NO: 321)
KYPSRTYYKQTCIVCNITTGENTLFKLAKSDGKHAEELLLEELEKLVPKDSLTITIFMN
DTPCSLAGHDCAGKFVQYLKTAKVDLTLYVTSLCESKKKDIHKGLAGLKQCTVKSP
NRDAWQEFIMDLNDWKKYEKQEKDGIEK
>K1QUE8_CRAGI/4-176
(SEQ ID NO: 322)
NLDHQDWDKAKSCLICKIDDNITVLEKTEGTHAEEFLLQELEKKVRNVTIFMNNSPC
STPNHTCADKLLKYLDASENVRMTMYVTRLYMIARDGHHAGLQRLKQHACEINAF
NKNWEELITKMEFKEYNVRSRKEED
>A0A0W0VFJ2_9GAMM/61-217
(SEQ ID NO: 323)
YGNSKEQLFMLAKIPELFPESRHAEENLILGFSGIEQFFPEQIRKIDIFLSHSPCSEQGVK
CSGSLYLNGCDQKLIAFFKKGNDLDRSLFLENSNVQVRYHRQFDSAVENFIIQAPELK
DWKN
>A0A0W0XR23_9GAMM/33-206
(SEQ ID NO: 324)
ISNELQVSAPKKKQRLFMLDNVPAVFPESNHAEENLIRRFPDVSQFFPGQIINITIFLSH
SPCCAEGEKHSDARPINGCNKKLSVFFEKGNALDASLFVNTKFKVQYLYRFNHTVEP
FIRQAPLLH
>A0A0W0S272_9GAMM/47-207
(SEQ ID NO: 325)
IGNELSKQDTPRKHQLFMIAKVPQLFPQHAQHAEENLIRGFPSINKFYPNQISKIDIFLT
HSPCSEEGGKHSSQNSVNGCDKKLTVFFKKGNSADVQLFNRNTKVKVYYNHKFDS
ATDEFIKEAPI
>A0A098GDC1_TATMI/41-209
(SEQ ID NO: 326)
IGNELKNSDLDRKQKLFMIGGVPQQFPRHSQHAEENLIRNFPNIKKFFPNQINTVEIFL
THSPCSSNGQKYSAQCYTNGCDKKLSAFFKKENYFDQQLFNRKVKVRIHYNHQFDP
SINHFIKEAPILK
>A0A0M0LPN3_9EUKA/47-262
(SEQ ID NO: 327)
ERFLNAPLVRKPNTILMLRVQSEEMYLCYTRGYANSLHAEQFMMEDPELLRLLQPV
RTLLMTLLMTQQPCHENAHASCTLRLLRWCKRERGIDLSIRIARVFRAADESARDGL
RLLMRAPVEVAMLTCEDWQHCDERLVAFRPWHEMED
>C1E186_MICCC/53-299
(SEQ ID NO: 328)
FFHCSPTPEARKTKGVVVAALRRGDLRSNSSHAEEYVVRDEELVRAVAPEDAGTLT
LYQRLQPCHGSSDNRGSCSDAVAGLHRELLGVSLRVAVSYTYRAHVRGFREGIRVF
AADGVTLEALNAEDWALCDEDYAAAFGGVFTAAVAHRRAMDE
>A0A0M0K2Z4_9EUKA/249-414
(SEQ ID NO: 329)
PPPNIFLAQTREREAETGLRLLLYMTYQPCHHSGGRPKEALGTCSESLRDFYIKELGV
ALELVLADVYKATEELHREGMRMLLAEGITMRAMQPADWDLCDPEWAKRGAGPF
SKH
>R1E5J9_EMIHU/273-492
(SEQ ID NO: 330)
CGKVVVAVALEHWRAGSSENVHAESFVIADETILSAVVAELTLYLSYQPCHHSGGR
GGGEARQSCTEELIAYHERELNVPLSVVVADLYKVMEELMREGIRRLMSPGMSMRA
TGEEDWALCDQSYERRGGSAFGPHVALRAKLD
>A0A0M0LPN3_9EUKA/390-614
(SEQ ID NO: 331)
RGRRDGPVIVARVLGQSADLYVAYARGRSENVHAEEFMLADPQLLALLDAGGARIL
RLYMSYQPCHHSGGRKTPEDARKSCSERLRAFYEAEMAISLELVVADLYKAMEDLM
RQGILLMVAPGVTMRATDENDWALCDPAYATRGGSAFSRH
>A0A0M0JB66_9EUKA/269-499
(SEQ ID NO: 332)
VDTRDKTKAQVIVARVLGQNADLYVAYARGRSENVHAEEFMLADPQLLALLDASG
ARILRLYMSYQPCHHSGGRKTPEDARKSCSERLRAFYEAEMAISLELVVADLYKAM
EDLMRQGILLLVAPGVTMRATDENDWALCDPAYETRGGSAFSRHE
>A0A183IJ21_9BILA/199-397
(SEQ ID NO: 333)
SCKSFDERGASTMVACLRTTNGTYQEYRATDNDDRHPEEIFHKDMLQARTYVLPLT
HIIVYLPTSPCFHQDCEPQCDVLDACAEQLAIVYRQAKKTDLKMSVKFLASYIGDLY
KQGITMMMNAGIDVEPLGMRDWIELVHNDSTYYFSWKGLLTSYIRQSEIYI
>A0A077Z854_TRITR/190-389
(SEQ ID NO: 334)
RKLQYRGSSALVAQLTETATYRAFVVSEKTIHVEQKFYHQLMDAANFTMPLREVIL
YLPTSPCFHQDCDPLCDVLDACAEALSICYKQIRREELQMTVKFLASYVGDLYKQGI
MSMMEAGLTVEPLNMKDWISLVSTAGTYYRDWENSLADYVMQTQLYI
>A0A085NPE0_9BILA/206-389
(SEQ ID NO: 335)
AQLTETATYRAFVVSEKTVHVEQKFYHHLMDAANFTMPLRELILYLPTSPCFHQDCD
PLCDVLDACAEALSICYKQIQREELQMTVKFLASYVGDLYKQGIMSMMEAGITVEPL
NMKDWISLVSTAGTYYRDWENSLADYVMQTQLYI
>A0A0V0XXU8_TRIPS/513-708
(SEQ ID NO: 336)
LNEQVQKWEHRGASVMVAKLSDQQVYADYFVGDGKPQHVEQIFYTELMNADQCQ
MATLRQICLFMPTSPCFHQDCEPQCDVLDACAETLAIVYKQLQNSDLQMTVKFLAS
YVGDLYKQGILCMLQAGISVEPLNRKDWQALVEPDHTRAWDSLLTNYAMQSQFYI
>A0A0VIMSE5_9BILA/488-683
(SEQ ID NO: 337)
LNEQVQKWEHRGASVMVAKLSDQQVYADYFVGDSKPQHVEQIFYTELMNADQCQ
MTTLRQICLFMPTSPCFHQDCEPQCDVLDACAETLAIAYKQLQNSDLQMTVKFLASY
VGDLYKQGILCMLQAGISVEPLNRKDWQALVESDHSRSWDSLLTNYAMQSQFYI
>E5S912_TRISP/108-302
(SEQ ID NO: 338)
PNEQVQKWEHRGASVMVAKLSDQHVYADYFVAETKPQHVEQIFYAELINADQCRM
TTLRQICLFMPTSPCFHQDCEPQCDVLDACAETLAIAYKQLQNPDLQMTVKFLASYV
GDLYKQGILCMLQAGISVEPLSRKDWYALVESDHTRGWDSLLTNYAMQSQFYI
>A0A183CD28_GLOPA/474-665
(SEQ ID NO: 339)
VPKHVQNCEHKHESGLIVTLGDGYIYGDFFHDSNPHVEEQLVAAIYDLSKYKVDLYE
IVIFVSKSPCFHQDCDPKCEVVDACAKLLGLLLSKVRKVDVRMTVKFLYPHLGDLY
KQGILSMLQSGIKVEPLLMKDWSAVMDWAGEYLQWGSHLDRAVAQSQAFI
>H3FBK6_PRIPA/51-311
(SEQ ID NO: 340)
FHVPKHVQSCEHKHESGLIVTLGEDYIYGDFYHESSPHVEEQLVAAIYDLSKYKVDL
YEIVIFVSKSPCFHQDCDPKCEVVDACAKLLGLLLSKVRKVDVKMTVKFLYPHLGDL
YKQGILCMLQAGIKVEPLLMKDWSAIMDWTGEYLQWNNHLDKAVAQSQS
>A0A0N5AZH9_9BILA/14-264
(SEQ ID NO: 341)
FHIPKHVQSCEQKHESGLIVTLGDDYVYGDFYHESNPHVEEQLVAAIYDLSKYKVDL
YEIMIYVSKSPCFHQDCDPKCEVVDACAKLLGLLLSKVRKVDIRMTVKFLYPHLGDL
YKQGILCMLQAGIKVEPLLMKDWSAVMDWSAEYLQWNNHLDKAVAQSQAFI
>A0A0B2VFT1_TOXCA/106-363
(SEQ ID NO: 342)
FHVPKHVQSCEQKHESGLIVTLGEDYIYGDFYHESSPHVEEQLVAAIYDLSKYKVDL
YEMVIYVSKSPCFHQDCDPKCEVVDACAKLLGLLLSKVRKVDVKMTVKFLYPHLG
DLYKQGILCMLQTGIKVEPLLMKDWSAVMDWSGEYLQWNNHLDKAVAQSQSF
>A0A158Q5D3_DRAME/117-306
(SEQ ID NO: 343)
IPKHVQSCEHKNESGLIVTLGDDYIYGDFYHESSPHVEEQLVAAIYDLSKYKVDLNEV
VIYVSKSPCFHQDCDPKCEVVDACAKLLGLLLSKIRKVDVRMTVKFLYPHLGDLYK
QGILCMLQAGIKVEPLLMKDWSTVMDWSGEYLQWNNHLDKAVAQSQSF
>A0A0N5CVT8_THECL/156-346
(SEQ ID NO: 344)
VPKHVQNCEHKNESGLIVTLGHDYVYGDFYHESSLHVEEQLAAAIYDLSKYKVDLF
EAVIYVSKSPCFHQDCDPKCEVVDACAKLLGLLLSKIRKVDVKMTVKFLYPHLGDL
YKQGILCMLQAGIKVEPLLLKDWSAVMDWTGEYLQWNNHLDKAVAQSQSF
>J0XJI9_LOALO/138-380
(SEQ ID NO: 345)
FHLPKHVQSCEHKNETGLILTLGDDYIYGDFYHESSLHVEEQLVAAIYDLSKYKVDL
FEAVIYVSKSPCFHQDCDPKCEVVDACAKLLGLLLSKIRKVDVKMTVKFLYPHLGDL
YKQGILCMLQAGIKVEPLLLKDWSAVMDWTGEYLQWNSHLDKAVAQSQSF
>A0A0K0EN59_STRER/201-391
(SEQ ID NO: 346)
VPKHVQNCEHKHESGLIVTLGEDYIYGDFYHESGPHVEEQLVAAIYDL
SKYKIELYEIVIFVSKSPCFHQDCDPKCEVVDACAKLLGLLLSKVRKVDVKMTVKFL
YPHLGDLYKQGILCMLQAGIKVEPLLMKDWSAIMDWAGEYLQWNNHLDKAVAQS
QSF
>H2W477_CAEJA/255-446
(SEQ ID NO: 347)
VPKHVQNCEHKHESGLIVTLGEDYVYGDFYHESGPHVEEQLVAAIYDLSKYTVDLH
EIQIFVSKSPCFHQDCEPKCEVVDACAKLLGLLLSKVRKVDVKMTVKFLYPHLGDLY
KQGILCMLQAGIKVEPLLMKDWCAIMDWSGDYLQWNNHLDKAVAQSQLFI
>A0A0K0DCY3_ANGCA/157-328
(SEQ ID NO: 348)
IPKHVQNCDHKHESGLIVSLGDDYIYGDFYHESGPHVEEQLVANIYDLSRYKVDLHEI
VIFVSKSPCFHQDCDPKCEVVDACAKLLGLLLSKIRKVDVKMTVKFLYTHLGDLYK
QGILCMLQAGIKVGFFSVLQKAVAQSQLFIN
>A0A158PLA6_ANGCS/132-323
(SEQ ID NO: 349)
IPKHVQNCDHKHESGLIVSLGDDYVYGDFYHESGPHVEEQLVANIYDLSRYKVDLHE
VVIFVSKSPCFHQDCDPKCEVVDACAKLLGLLLSKIRKVDVKMTVKFLYTHLGDLY
KQGILCMLQAGIKVEPLLMKDWCAIMDWSGDYLQWNNHLDKAVAQSQLFI
>U6NTW9_HAECO/157-347
(SEQ ID NO: 350)
VPKHVQNCDHKHESGLIVSLGENYIYGDFYHESGPHVEEQLVANIYDLTKYNVELHE
IVIFVSKSPCFHQDCDPKCEVVDACAKLLGLLLSKVRKVDVKMTVKFLYPHLGDLY
KQGILCMLQAGIKVEPLLMKDWCAIMDWSGDYLQWNNHLDKAVAQSQLFI
>A0A158R0Z7_NIPBR/52-302
(SEQ ID NO: 351)
FSVPKHVQNCDHKHESEDYIYGDFYHESGPHVEEQLVANIYDLTKYNVELHEIVIFVS
KSPCFHQDCEPKCEVVDACAKLLGLLLSKVRKVDVKMTVKFLYPHLGDLYKQGILC
MLQAGIKVEPLLMKDWCAIMDWSGDYLQWNNHLDKAVAQSQLFI
>A0A0C2D4B7_9BILA/130-292
(SEQ ID NO: 352)
VPKHVQNCDHKHESGLIVSLGEDYIYGDFYHESGPHVEEQLVANIYDLSKYNVELHE
IAIFVSKSPCFHQDCDPKCEVVDACAKLLGLLLSKVRKVDVKMTVKFLYPHLGDLY
KQGILCMLQAGIKVCGHFFVCFAYLFV
>A0A183IMD0_9BILA/141-363
(SEQ ID NO: 353)
FAVTKNTKQCGQKNETAAIVTLGGGNIEFHHESSLHPEEQLFTALMELSEYQVNLEE
VVLYCSKSPCYHQDCNPLCEVIDACAKLLVLLLYKVRTVDVNLTVRFLYPHLGDLY
KQAIMYMLQHGINVEPLLMSDWSAIVEWGCNYLDWNEHLDQAVARGQS
>A0A0N5DEG0_TRIMR/83-275
(SEQ ID NO: 354)
FLVSRSTQQCEHKHESAIVVTLGEDYVYIEYAHESGLHCEEQLMNALEELAMYTVNL
YEVVVYTSRSPCFHQNCEPRCAVIDACSKLLSLFLLKLRRVDLRMTVRFLFPHLGDL
YKQGILCMLQHGIKVEPLLMKDWSAIMDWSGDYLAWNQYLDHAVAKSQSF
>A0A085LQT5_9BILA/165-355
(SEQ ID NO: 355)
ISRTTQHCEHKHESAIVVTLGEDYVYVEYLHESSLHCEEQLLYALEELAKYAINLYEV
LVYTSRSPCFHQNCEPRCAVIDACSMLLSLFLFKLRRVDLRMTVRFLFPHLGDLYKQ
GILCMLQHGIKVEPLLMKDWSGIMDWAGDYLTWNQHLDQAVAKSQSF
>A0A077ZDL7_TRITR/175-365
(SEQ ID NO: 356)
VSRTTQHCEHKHESATVVTLGEDYVYVEYLHESSPHCEEQLLFALEELAKYAINLYEI
LVYTSRSPCFHQNCEPRCAVIDACSMLLSLFLFKLRRVDLRLTVRFLFPHLGDLYKQG
ILCMLQHGIKVEPLLMKDWSGIMDWAGDYLAWNQHLDQAVAKSQSF
>E5SVP6_TRISP/124-300
(SEQ ID NO: 357)
SRQGQQCEQKHESVILVTLGEDYVYVEFCHESSRHCEEQLAIALHELVNRSPCFHQD
CEPRCDVIDACSKLLALLLTKVRKADIRMTVRFLFPHLGDLYKQGILCMLQHGIKVE
PLLMRDWSAIMDWAGDYLGWNEHLDQAVAKSQSF
>A0A0VIMU51_9BILA/171-360
(SEQ ID NO: 358)
SRQGQQCEQKHESVILVTLGEDYVYVEFCHESNRHCEEQLAIALHELVKYKINLYEIL
IYSSRSPCFHQDCEPRCDVIDACSKLLALLLTKVRKADIRMTVRFLFPHLGDLYKQGI
LCMLQHGIKVEPLLMRDWSAIMDWAGDYLGWNEHLDQAVAKSQSF
>A0A067QK96_ZOONE/50-213
(SEQ ID NO: 359)
WTAFYINGRPKLKKCITLCHVVFNETATAEQWEISYSHGPHAEIKVLRNIKARELCLG
YTRIVTLFLSYSPCANCANFIIEFSRTRPQCTVYIRFTCLFRHPEEIHRDGLRRLNAPGIS
LGVFTVYEWRRLAEAGMPFRPWDSKWK
>H3B7Z9_LATCH/181-343
(SEQ ID NO: 360)
FYGKFNNTRKNGRNMLCFSLEGENKPWKWGYAHNSKHAENIVLREVASYKPFLNH
FYISYGPCSNCCDKILDFLQKFEKIKIMIKISRLYKDESSVFQNSIKKLHQMGVSVQVM
NRGDFEQCFKGFVQGDFQPWPALEPTSEKCAANLEA
>H3B7Z9_LATCH/4-168
(SEQ ID NO: 361)
FEAEFNNTVNSFRTLLCFSLQQENKTWNWGYAHNNDSHAEILVLREIEKYEKADHEI
RQRVTLYVTCSPCNRCCTKILEFFQRFQRFDMDIKISKIYDLDSLQDLKQLGVSLKVM
DSSDFKECFDLFVHTAEFEPWPGLEEKTKQLNAVFL
>W5M7A0_LEPOC/25-192
(SEQ ID NO: 362)
FYQEFCNTLRTCRTLLCFSLCQSTKIWDWGYAYNKGSHAESMVLEEIKTFQNQDKTL
KYTLTLYMSFSPCNECCYRLATYAKLERRIKINVMFSKLYFPEHRKIQKGLQYLECA
GVSLKVMEKQDFVTCFYLFVTEHAFQEWHCLDDMTKQYSSTLQA
>G1KV46_ANOCA/8-175
(SEQ ID NO: 363)
FEENFNNTVLVRKTLILFSFKKRSSIWKWGYAYNDGQHAECLVLSNIEQFENQIKGK
YKMTFFMSHTPCHKCSDKIVSFLASRKGLSMKIKASRPYFLNEGRKGLYLLKRIGVL
LKMMDRTDFEECFYLFVHPLTFTPWSDLDEQSKKNMDDLAA
>G3VS78_SARHA/14-179
(SEQ ID NO: 364)
FYEHFCNIKTPHQTLLCFSLKEDDKTWKWGHAYNNGYHAEILVLREIEDYANILNAT
KYTITWFLSYSPCHCCCDEITNFLMKFQKIEFNIKAARPYYFDNDKNQKGLKILKKLG
VLIKMMDFTDYEECLYLFVDPCKFTAWPDLEVQSIANKMIFHH
>K7E403_MONDO/13-175
(SEQ ID NO: 365)
FHRNFSSTKASHQTLLCFGLKEEDKTWKWGHAYNNGCHAEILVLREIENYTNIPNAA
KYNITWFLSYSPCHSCCDKIINFFMNSQKIEFNIKAAKPYQFSNDQNPKGIKMLNKLGI
LFKMMEYSDFEECFYLFVDPCKFTAWPDLEAQSIANIT
>A0A091G380_9AVES/2-125
(SEQ ID NO: 366)
LFEAGGYLDAVTCENIRCIILYSSYSPCNEVPHCCVSKIYNFSLKYPEITLCIYFSQLYH
TQCPSAREALRSLWSPRVTLQRPPGELWPSFVCGGSLSHPLRTS
>A0A091WIF8_OPIHO/3-124
(SEQ ID NO: 367)
FEVGGYLDAVVYKNIRCIILYANYSPCNEAYHCCISKIYNFLLKYPEITLCIYFSRLYH
TEFPTAQEALRSLSSPRVTLQRLPAGTQHYFVYGGPLYHPSRTL
>A0A099ZXL0_CHAVO/4-126
(SEQ ID NO: 368)
EVGGYLDTVVYENIRCIIVYSNYSPCNEAYHCCVSKIYNFLLRYPEITLCIYFSKLYHT
EFPTSREALRSLSSPQVTLQRLPGGAWHYFVYGGSLYHPSRTL
>A0A091KLV2_9GRUI/4-128
(SEQ ID NO: 369)
EVGGYLDAVAYENIRWIILYSNYSPCNEAHHCCVSKIYNFLLKYPEIRLCIYFSQLYHT
EFPTAREALRSLSSPRVILQRLPGGMWHYFVYGGSLYHLSRTLQQ
>A0A093ITV6_FULGA/4-127
(SEQ ID NO: 370)
EVGGYLDAVAYENIRCIILYSNYSPCNEAYHYCISKIYNFLLKYPEITLCIYFSQFYHTE
FPTAREALRSLSSPRVTLQRLPGGARRYFVYGGALYHPSRTLR
>A0A087QVF4_APTFO/2-125
(SEQ ID NO: 371)
LFEMGGYLDAVAYENIRRIILYSTYSPCNEAYHCCISKIYNFLLKYPEITLCIYFSQLYH
TESPTAREALRSLSSPRVTLQRLPAGAQRYFVYGGSLYHPSRTL
>A0A091LC91_CATAU/4-127
(SEQ ID NO: 372)
EVGGYLDAVAYENMGCIILYSNYSPCNEAYHCCVSKIYNFLLKYPEITLCIYFSQLYH
TEFPTAREALRSLSSPRVTLQRLPGGAWRYFVYGGSLYHPSRTLR
>A0A091UXX9_PHALP/7-125
(SEQ ID NO: 373)
GYLDAVACENIGCIILYSNYSPCNEAHHFCISKIYNFLLKYPEIALCIYFSQLYHTEFPS
AREALRSLSSPRVTLQRLPGGTWRYFVYGGSLYHPPRTL
>E1BTD6_CHICK/49-244
(SEQ ID NO: 374)
RAFGFPCRAPQTHHLLFYELKSFSGTVVQKGHATSEDNHPESMLFEADGYLDAVAY
RNIGCITLYSNYSPCNEAYHCCVSKIYNFLLKYPEITLCLYFSQPYHTEFPTARQALHS
LASPQVTLQPLPTGTWCHFVYGGSLQHPPGTLRQNPHQINNFRG
>G3UV61_MELGA/48-245
(SEQ ID NO: 375)
LRAFGFPCRAAQTHHLLFYELRSFSGTVVQKGHASSEDSHPESMLFEADGYLDAVA
YGNTGCITLYSNYSPCNEAYHCCISKIYNFLLKHPETTLCLYFSQPYHIEFPTARQALR
SLASPWVTLQPLPMGTRCHFVYGGSLQHPPGTVGQNPHQINNLRG
>A0A093DVS1_9AVES/2-85
(SEQ ID NO: 376)
LFEVGSYLDAVAHENVGCIILYSNFLPCNEAYHCCISNIYNFLLKYPEITLCIYFSQLYH
TQFCATRKALQSESSEN
>A0A091Q6M3_LEPDC/5-125
(SEQ ID NO: 377)
VGGYLDAVAYGNIGCIILYSNYSPCNEAYHCCISKIYNFLLKYQEIILRIYFSQLYHTEF
PTACRALWSLSSPRVTLQRLPGGAWHYFVHGGSFYHPSRTL
>A0A093HKT8_STRCA/2-124
(SEQ ID NO: 378)
LFEAGGYLDSVTYESIGHIILYSNYSPCNEADHCCISKIYNFLIKHPEVTLCIYFSQLYH
TEFPTAREALRSLSSPHVTLYPLSGGIRHYFVYGRSLYHPSRTL
>A0A099Z1L5_TINGU/2-122
(SEQ ID NO: 379)
LFEAGGYLDAVTYEDIGYIILYSNYSPCNEADHCCLSKIYSFLTKYPQVTLCIYFSKLY
HIELPTAHEVLKSLSSPHVTLHPLCGGIWHYFVHGGSLYHPSR
>H9GLZ8_ANOCA/51-241
(SEQ ID NO: 380)
FHQAFGFPYTHQNKHLLFYEVRHFSGKLMQKGHATNEDIHPESMLFETGGYLDSIN
WENVMYIILYSNYSPCNEAEQCCISKIYHFLMKYPGITLCIYFSQLYHTELPISCEALQS
LASPQVTLNPLCGGLWHSFVTSQACYHPIRALRQNAQQINNITG
>M7B925_CHEMY/46-257
(SEQ ID NO: 381)
FHKAFGFPYMPQNKHLIFYELRSFSGTLVQKGHATNKNIHPESMLFEMGGYLDALD
YDSIRYIILYSNYSPCNEAEHCCISKIYNFLTKYPDITLCIYFSQLYHMEFPVSCEALRS
LASPHVTLNPLCGGVWHSFVSGEALYHPARALQQNSYKINNITG
>H3B3J0_LATCH/47-227
(SEQ ID NO: 382)
FYEAFGFPYTPRNKHLLFYELRNISGTLIQKGQATNLNLHPESTLFDLDGYLDSIIYDN
ISYITLYSNYSPCNESNHYCIGKMYDFLISYPSTRLDIYFSQLYHTDFPESREALRSLAG
PRVTISPISGGTWLSFVNGQALYNPTRAFKHNAY
>F6ZR34_XENTR/47-234
(SEQ ID NO: 383)
FYEAFGFPYTPENKQLIFYEVKDFSGTNIQKGQVTNSNIHAESILFEDSGYLDALHHGS
VGYITLYANYTPCNEYGHYCISKMYNFLLKYEDTRLDIYFSQLYHVESPAARQALRS
LASPRVTVNPLSEGIWQTFAKGLSFYEPARASSSNASTIHLITG
>F6VU78_ORNAN/48-231
(SEQ ID NO: 384)
FYQTFGFPHMPQPEHLTFYELKTFSGAPVQKGQATSQSIHPESMLFEEGGYLDSVYD
DSIGHIILFSNYTPCNEAGHCCISQMFDFLMTYPDITLSIYFSQLYHTEFPASRKALRSL
ASPRVSVNPISGGIWHAFVSGGALRHPSRALGHNAYEINA
>G3WLB3_SARHA/47-227
(SEQ ID NO: 385)
FYQIFGFPYSPQTQHLTFYELKTSSGSLVQKGHASGEDTHPESMLFEMDSYLEAVNN
DNIEHVFLYSNYCPCNEANHCCISKMYNFLMRYPAISLNIYFSQLYHTEFPVSREALR
SLASPQVTVNPMSGGIWHYFVSDEALHQPARTLRHNAYE
>H0VBH8_CAVPO/47-214
(SEQ ID NO: 386)
FHQIFRFPYTPSTKHLTFYELKTFSGSLVQKGHASNGHTHPESMLFEMNGYLDSANN
SSIKHIILYSNNSPCNEANHCCISKMYNFLTMYPDVTLSVYFSHLYHTGFPASREALRS
LASPQVTLSPISGGIWHCFVTGGAAFQP
>G5B4444_HETGA/47-231
(SEQ ID NO: 387)
TKHLTFYELKTSSGTLVQKGHASNGDTHPESMLFEMNGYLDSASN
STIKHIILYLNNFPCNEANHCCISKMYNFLMMYPDITLSIYFSQLYHTKFPTSREALRS
LASPQVTLSPISGMIWHSFVMGGPAFQPGRALRHNAYE
>ABEC4_MOUSE/47-263
(SEQ ID NO: 388)
FHQTFGFPWTPQTKHLTFYELRSSSKNLIQKGLASNGHNHPEAMLFEKNGYLDAVH
NSNIRHIILYSNNSPCNEAKHCCISKMYNFLMNYPEVTLSVFFSQLYHTEFPTSRKALQ
SLASPQVTLSPICGGLWHAFVSNGSVPQPGRILRYNTYEINSIIA
>ABEC4_RAT/47-256
(SEQ ID NO: 389)
FHQTFGFPWTPQTKHLTFYELRSSSGNLIQKGLASNGHTHPESMLFERDGYLDSLHD
SNIRHIILYSNNSPCDEANHCCISKMYNFLMNYPEVTLSVFFSQLYHTEFPTSREALRG
LASPQVTLSAISGGIWQSFVSGGLAVRPGRTLRYNAYEINCITE
>F7D911_HORSE/47-232
(SEQ ID NO: 390)
FYQIFGFPYMPQTKHLTFYELKTTSGSLVQKGHASSGNTHPESMLFELNGYFDSANN
DSIRHIILYSNNSPCNEANHCCISKMYNFLIMYPHVTLSIYFSQLYHTEFPASREALRSL
ASPQVTLSPISGGIWHSFVSGGPVFQPGRALRHNAYEINAI
>GINXW1_MYOLU/47-231
(SEQ ID NO: 391)
FYQIFGFPYTPQTKHLTFYELKTSTGGLVQKGHASSTSTHPESMLFETNGYFDWANN
GGIRHIILYSNHSPCNEADHCCISKMYNFLTTYPDVTLSIYFSQLYHTDFPASREALRS
LASPKVTLSPISGGIWHFFVSGGSVFQPGRALRHNAYEINA
>F1S657_PIG/47-239
(SEQ ID NO: 392)
FYQLFGFPYTPQTKHLAFYELRPSSGSLVQKGHASSGDTHPESMLFERNGYFDSANN
NGIRHIILYSNNSPCNEANHCCISKMYNFLRMYPDVTLSIYFSQLYHTEFPASREALRS
LASPQVTLSPISGGIWHSFVSGGWLFQPGRALRHNAYEINAITG
>S9WFU5_CAMFR/54-199
(SEQ ID NO: 393)
FYQLFGFPYVPQTKHLTFYELKTSPGSLVQKGHENSGDTHAESMLSEMNGYFDSAN
HQGIRHIILYSNNSPCDEANHCCTSKMYNFLTMYPDVTLSIYFSQLYHTEFPASREAL
RSLASPQVTVSPP
>H0WM53_OTOGA/47-232
(SEQ ID NO: 394)
FYQIFGFPYISQTKHLTFYELKTSSGSLVQKGHASRGDTHPESMLFEMNGYLDSANN
DGIRHIILYSSNSPCNEANHCCISKMYNFLKVYPDVTLSVYFSQLYHTEFPASREALRS
LASPQVTLSPISGGIWHSFVSGGSVFQPGRALRQNAYEINAI
>G3MWJ4_BOVIN/47-233
(SEQ ID NO: 395)
FYQIFGFPYTPPTKHLTFYELKTSSGSLVQKGHASSGDTHPESMLFEVNGYFDSANND
CIRHIILYSSNSPCNEANHCCISKMYTFLAKYPDITLSVYFSQLYHTEFPASREALRSLA
SPQVTLSPISGGIWYSFVSGGAVFQPGRALRHNAHEINAIT
>W5P5G7_SHEEP/47-235
(SEQ ID NO: 396)
FYQIFGFPYTPPTKHLTFYELKTSSGSLVQKGHASSGDTHPESMLFEMNGYFDSASND
GMRHIILYSSNSPCNEANHCCISKMYTFLAKYPDITLSIYFSQLYHTEFPASREALRSL
ASPRVTLSPISGGMWYSFVSGGAVFQPGRALRHNAHEINAITG
>G1L6U8_AILME/47-234
(SEQ ID NO: 397)
FYQIFGFPYTPQTKHLTFYELKTSSGSLVQKGHASSGNTHPESMLFEMNGYFDSANN
NAIRHIILYSNNSPCNEANHCCISKMYNFLILYPDITLSIYFSQLYHTEFPASREALRSL
ASPQVTLSPISGGMWHSFVSGGSVFQPGRALRHNAYEINAITG
>M3YK05_MUSPF/47-239
(SEQ ID NO: 398)
FHQIFGFPYVPQTKHLTFYELKTSSGSLVQKGHASSENTHPESMLFEMNGYFDAAKN
NAIRHIILYSNNSPCNEANHCCISKMYNFLIMYPDVTLSIYFSQLYHTEFPASREALRS
LASPQVTLSPISGGIWHSFVSGGPVFQPRRALRHNAYEINRITG
>M3WE63_FELCA/47-233
(SEQ ID NO: 399)
FYQIFGFPYAPQTKHLTFYELKTSSGSLVQKGHASSGNTHPESMLFEMNGYLDSANN
NAIRHIILYSSNSPCNEADHCCISKMYNFLIMYPDVTLSIYFSQLYHTEFPASREALRSL
ASPQVILSPISGSIWHSFVSGGSVFPPGRAQRHNAYEINAIT
>E2RD46_CANLF/47-236
(SEQ ID NO: 400)
FYQIFGFPYVPQTKHLTFYELKTSSGSLVQKGHASSGNTHPESMLFEMNGYFDSANN
NTIRHIILYSNNSPCNEANHCCIGKMYNFLITYPDVTLSIYFSQLYHTEFPASREALRSL
ASPRVTLSPISGGIWYSFVSGGSVFQPGRALRHNAYEINAITG
>L9L463_TUPCH/47-264
(SEQ ID NO: 401)
FYQIFGFPYTPQTKHLTFYELKTSSGSLVQKGHASSGQIHPESMLFEMNGYLDLANN
GGIRHIVLYSNHSPCNEAHHCCISKMYNFLITHPDITLSIYFSQLYHTEFPTSREALRSL
ASPRVTLSPISGGIWHSFVSGGSVFQPGRALRHNAYEINAITG
>A0A0D9SA64_CHLSB/47-231
(SEQ ID NO: 402)
FCQIFGFPYTPQTKHLTFYELKTSSGNLVQKGHASSGYIHPESMLFEMNGYLDSANN
DNIRHIILYCNNSPCNEANHCCISKVYNFLITYPGITLSIYFSQLYHTEFPASREALRSL
ASPRVVLSPISRGIWHSFVSGGSVFQPGRALRHNAYEINA
>F7GWZ1_CALJA/47-228
(SEQ ID NO: 403)
FCQIFGFPYTHQTKHFTFYELKTPSGSLVQKGHASSGYIHPESMLFEMNGYLDSASND
SIRHIILYSSNSPCNEANHCCISKMYNFLITYPGVTLSIYFSQLYHTEFPASREALRSLAS
PQVILSPISGGIWHSFVSGGSVFQPRRALRHNAYE
>H2N4F1_PONAB/47-228
(SEQ ID NO: 404)
FCQIFGFPYAPQTKHLTFYEVKTSSGSLVQKGHASSGDIHPESMLFEMNGYLDSANN
DSIRHIILYSSNSPCNEANHCCISKMYNFLITYPGVTLSIYFSQLYHTEFPASREALRSL
ASPRVVLSPISGGIWHSFISGGSIFQPGRALRHNAYE
>G3R253_GORGO/47-228
(SEQ ID NO: 405)
FCQIFGFPYTPQTKHLTFYELKTSSGSLVQKGHASSGYIHPESMLFEMNGYLDSANND
SIRHIILYSNNSPCNEANHCCISKMYNFLITYPGITLSIYFSQLYHTEFPASREALRSLAS
PRVVLSPISGGIWHSFISGGSVFQPGRALRHNAYE
>K7G199_PELSI/28-181
(SEQ ID NO: 406)
DPSVLRRVQYLLYEVKWSNSRKLTQCCHSTRTEHAEIYFLEDVFHRQRYDPSDHCSL
TWYMSWSPCGECCKAIRDFLKEQPNVNLVIYVARIYCHEEENNRQGLRSLVNIVTIRI
MDLPVYSYCWRTFVCDEDKDEDYWPRHFAPWIMLYS
>K7G211_PELSI/1-118
(SEQ ID NO: 407)
HAEIYFLKDVFNRQRNDPSDHCSLTWYMTWSPCGECCKAIRDFLKEQPNVNLVIYV
ARIYCHEEENNRQGLRSLVNIVTIRIMDLPVYSYCWRTFVCDEDNFPGSPEGSHHKS
MLLT
>A0A09318Y9_STRCA/1-74
(SEQ ID NO: 408)
ITLYLSWSPCRNCCYEMQYFLKKHPNVNICIYLARLYYTEDEEICKALKDLSEKVIISV
MKIEDYIYCWKTFV
>A0A091MHU6_9PASS/1-63
(SEQ ID NO: 409)
ITWYLSWSPCVNCCYKIRDFLNRHSYVTIRIYVARLCYRGFHRNRKGLRNLVSLRVT
VNVME
>A0A093S6Z3_9PASS/1-65
(SEQ ID NO: 410)
ITWYLSWSPCVNCCNEILDFLERHENVNIDIHVARLYFKDSKRTHRALKELARSTVSI
NVMNME
>R4GDA3_ANOCA/18-173
(SEQ ID NO: 411)
QRNFDPREFPECTLLLYEIHWDNNTSRNWCTNKGLHAEENFLQIFNEKIDIRQDTPCSI
TWFLSWSPCYPCSQAIIKFLEAHPNVSLEIKAARLYMHQIDCNKEGLRNLGRNRVSIM
NLPDYRHCWTTFVVPRNEDYWPQDFLATNYSREL
>A0A091ETZ4_CORBR/1-64
(SEQ ID NO: 412)
ITWYLSWSPCMTCCYIIRNFLVRHPNVNIEIHVARLYNTRWAGTRRGLRELARLRVTI
DVME
>U3JXR8_FICAL/12-171
(SEQ ID NO: 413)
FDPRTYPSETYLLCELQWGGSGRFWIHWARNDEDSHVEHYFLEQIFEPRSYSVCDIT
WYLSWSPCANCCDIIQEFLEEQHNVNLDIRVARVYNEHIRENRAALRQLANFQAAIR
AMDVEDYMYCWDTFLQQGGYFDFTAGSFRSAVERTRLRLE
>H0ZSB5_TAEGU/1-76
(SEQ ID NO: 414)
DMTWYLSWSPCGECCDIIQDFLEEQPNVNINIRVARLYYTDRASNRKGLRELASSPV
TLEIMDAEDYNYCWETFI
>A0A091R868_9GRUI/1-65
(SEQ ID NO: 415)
ITWYLSWSPCANCCYEIVDFLQRHSYVNIKIFVARLYYIDRERNRQGLRDLMNSAVTI
DVMDIE
>A0A091QH63_MERNU/1-65
(SEQ ID NO: 416)
ITWYLSWSPCANCCYRIVQFLMKHSYVSIDIRVARLYFIEDETTRQGLEELVSCAVRL
TVMDTE
>A0A0911KM1_CALAN/1-70
(SEQ ID NO: 417)
ITWYLSWSPCACCCCKIQDFLKMNSYVNTDIDVAQLYGNYQEQNCQGLKNLKSLAV
TIAVMRIEDKISC
>A0A094K7N8_ANTCR/1-65
(SEQ ID NO: 418)
ITWYLSWSPCANCCRKIRNFLKKHSYVYIDIYVARLYYIDDEENRRGLRNLQSLDVTI
AVMEIE
>A0A091GQ55_BUCRH/1-65
(SEQ ID NO: 419)
ITWYLSWSPCADCCCKIVNFLKKHSYVNMRIYVARLYYPEYETNRRGLKNLRNLAV
PIAVMEIE
>A0A151P7C9_ALLMI/18-177
(SEQ ID NO: 420)
FEKNYKPIDGTKEAHLLCEIKWGKYGKPWLHWCQNQRNIHAEDYFMNNIFKAKKH
PVHCYVTWYLSWSPCADCASKIVKFLEERPYLKLTIYVAQLYYHTEEENRKGLRLLR
SKKVIIRVMDISDYNYCWKVFVSNQNGNEDYWPLQFDPWVKENYSRL
>A0A093CT04_TAUER/1-65
(SEQ ID NO: 421)
ITWYLSWSPCARCCYKILDFLKEHSYVNLHIYVARLYCIEDEKTRRGLKKLNSLEVTI
AVMEEE
>A0A091V1W3_PHORB/1-65
(SEQ ID NO: 422)
ITWYLSWSPCAKCCYEILNFLKKHPNVNIDIYVARLYDIEKEKTRQGLKNLVRLPVTI
AVMEME
>A0A091V9F4_NIPNI/1-65
(SEQ ID NO: 423)
ITWYLSRSPCAKCCYEILDFLNKHSNVNIDIYIAQLYKIKNEENCQGLRNLVSLAVTIA
VMEIE
>A0A151P6H5_ALLMI/36-174
(SEQ ID NO: 424)
SELTWGGRPYKHWYENTEHCHAEIHFLENFSSKNGSCIITWYLSWSPCAECSARIADF
MKENTNVKLNIHVARLYLHDDKHTRQGLRYLMKMKVTIQIMTIPDYKYCWNTFLE
DDGEDESDDYGGYAGVHEDEDESD
>A0A099ZZR5_CHAVO/1-65
(SEQ ID NO: 425)
ITWYLSWSPCAECCLKILNFLEENSNVNIDIHIARLYRIQDERNRQGLRELVSSEVTIA
VMGIE
>A0A093J615_FULGA/1-65
(SEQ ID NO: 426)
ITWYLSWSPCAKCCRKILNFLKMHSNVKIDIYVARLYYIEDEKNRQGLKKLVSLAVK
IAVMEIE
>A0A091PTE0_LEPDC/1-75
(SEQ ID NO: 427)
VTWYLSWSPCVNCCRKILNFLKKHSNVNIDMHVARLYYIEDERIRQGLKNLVSLAVT
IAVMEIEDYTYCWKNFI
>A0A093JP51_EURHL/1-65
(SEQ ID NO: 428)
ITWYLSWSPCAHCCRKILNFLKRHSDVNIHIYIARLYYIENEEIRQGLKNLVSLEVKIA
VMETE
>A0A091RWG2_NESNO/1-65
(SEQ ID NO: 429)
ITWYLSWSPCVNCCRKILKFLKQHSYVNIKIYVARLYYIDDDEIRQNLKNLVSLVVTI
AVMDIE
>A0A0Q3WRD0_AMAAE/72-248
(SEQ ID NO: 430)
LKYHFDPREVXRDTYLLCILRWGETGTPWSHWVKNRYHAEVYFLEKIFQTRKSSKNI
NCSITWYLSWSPCAKCCRKILNFLKKHSYVSIKIHVARLFRIDDKETXQNLKNLGSLV
VTVSVMEXEDYTNCWKTFIRGHADGDSWIDDLKSEIRKNRLKFQ
>A0A091PR75_HALAL/1-65
(SEQ ID NO: 431)
ITWYLSWSPCADCCHKILKFLKKHSNVNIDIHVARVYYAEDEKVRQGLKNLVSLAV
TIAVMETK
>A0A087QNJ4_APTFO/1-65
(SEQ ID NO: 432)
ITWYLSWSPCADCCRKILNFLKKNSNVNIDIYVARLYYTEDEKIRQGLQNLVSLAVTI
AVMETE
>A0A087VRL2_BALRE/1-66
(SEQ ID NO: 433)
ITWYLSWSPCADCCHKILNFLKRHSNVNIDIYVARLYYIEDEEIRQCLKNLVSLAVTIA
VMKIE
>A0A093F6R6_GAVST/1-69
(SEQ ID NO: 434)
ITWYLSWSPCANCCRKILRFLRKHSNVNIDIHVARLYYIEDENIRQGLKSLVNLAVTI
AVMEIEGKVF
>A0A091TH63_PHALP/1-65
(SEQ ID NO: 435)
ITWYLSWSPCADCCHKILNFLKKHSNVNIIIYVARLYYKEDEKIRQGLKNLVNLAITIA
VMEIE
>A0A093QYH8_PHACA/1-65
(SEQ ID NO: 436)
ITWYLSWSPCEECCCKILNFLKKHSNVSICIYVARLYHIEDEKIRQGLKNLVNFTVTV
AVMGIE
>Clipboard_Contents
(SEQ ID NO: 437)
FEVFFDPRELRKETCLLYEINWGGRHSIWRHTSQNTNKHVEVNFIEKFTTERYFCPNT
RCSITWFLSWSPCGECSRAITEFLSRYPHVTLFIYIARLYHHADPRNRQGLRDLISSGV
TIQIMTEQESGYCWRNFVNYSPSNEAHWPRYPHLWVRL
>L5KGJ8_PTEAL/30-187
(SEQ ID NO: 438)
FEVFFDPRELRKEACLLYEIQWGTSHKIWRNSGKNTTKHVELNFIEKFTSERHFCSSV
SCSIIWFLSWSPCWECSKAIREFLSQRPTVTLVIFVSRLFQHMDQQNRQGLRDLINSG
VTIQIMRASEYDHCWRNFVNYPPGKEAHWPRYPPLWMKLYA
>L5LUG3_MYODS/37-228
(SEQ ID NO: 439)
IFDPRELRKEACLLYEIKWGTGHKIWRHSGKNTTRHVEVNFIEKITSERQFCSSTSCSII
WFLSWSPCWECSKAITEFLRQRPGVTLVIYVARLYHHMDEQNRQGLRDLVKSGVTV
QIMTTPEYDYCWRNFVNYPPGKDTHCPIYPPLLMKLYALELH
>F6WR88_HORSE/9-168
(SEQ ID NO: 440)
FEAFFDPRELRKEACLLYEIKWGMSHNIWRYSGKNTTKHVEINFIEKFTSERHLRPSIS
CSIVWFLSWSPCWECSKAIREFLSQHPNVTLVIYVARLFQHMDRLNRQGLRDLINSG
VTIQIMRTSEYDHCWRNFVNYPPGKEAHWPRYSLLWMKLYALEL
>F1SLW4_PIG/25-176
(SEQ ID NO: 441)
VFFDPRELRKETCLLYELQWGRSRDTWRHTGKNTTNHVERNFLAKITSERHFHPSVH
CSIVWFLSWSPCWECSEAIREFLDQHPSVTLVIYVARLFQHMDPQNRQGLRDLVNHG
VTIQIMGAPEYDYCWRNFVNYPPGKEAHWPRFPPVWMT
>W5NVH9_SHEEP/27-179
(SEQ ID NO: 442)
FDPRNFCKEAYLLYEIQWGNSRDVWRHSGKNTTKHVERNFIEKIASERHFRPSISCSIS
WYLSWSPCWECSKAIREFLNQHPNVTLVIYIARLFQHMDPQNRQGLKDLFHSGVTIQ
VMRDPEYDYCWRNFVNYPQGKEAHWPRYPPLWMNLYA
>M3WB96_FELCA/23-178
(SEQ ID NO: 443)
FEVFFDPRELRKEACLLYEIKWGTSHRIWRNSGRNTANHVELNFIEKFTSERHFCPSV
SCSITWFLSWSPCWECSKAIRGFLSQHPSVTLVIYVSRLFWHLDQQNRQGLRDLVNS
GVTVQIMRVPEYDHCWRNFVNYPPGEEDHWPRYPVVWMKL
>F1PUJ5_CANLF/23-183
(SEQ ID NO: 444)
FEGFFDPRELRKETCLLYEIQWGTSHKTWRNSGKNTTNHVEINFMEKFAAERQYCPS
IRCSITWFLSWSPCWECSNAIRGFLSQHPSVTLVIYVARLFWHTDPQNRQGLRDLINS
GVTIQIMTVPEYDHCWRNFVNYPPGKEDHWPRYPVLWMKLYALELH
>I3N301_ICTTR/12-175
(SEQ ID NO: 445)
FEVFFNPGVLRKETCLLYEIQWGTSRKIWRNSSKNTTNHVEVNFIEKFTAERHFCPSIS
CSITWFLSWSPCWECSKAIREFLSQHPNMTLVIYTARLFQHMDQQNRQGLRDLINSG
VTIQMMTVSDSLTCWLHFLSHRVYVILMKKCGNLV
>G1TVM9_RABIT/50-205
(SEQ ID NO: 446)
FEVFFDPQELRKEACLLYEIKWGASSKTWRSSGKNTTNHVEVNFLEKLTSEGRLGPS
TCCSITWFLSWSPCWECSTAIREFLSQHPGVTLVIFVARLFQHMDRRNRQGLKDLVT
SGVTVQVMSVSEYCYCWENFVNYPPGKAAQWPRYPPRW
>H0VV31_CAVPO/20-179
(SEQ ID NO: 447)
FEASFDPRQLQKEACLLSEVRWGASPRTWRESGLNTTSHVEINFIEKFTSGRSLRPAIR
CSVTWFLSWSPCWECARAIREFLHQHPNVSLVIYVARLYWHVDEQNRQGLRDLVTS
GVRVQIMSDSEYSHCWRNFVNFPPGQEAGWPRFPPMWTTLYA
>H0W6W5_CAVPO/16-179
(SEQ ID NO: 448)
FEAYFDPRQLRKEACMLSEVRWGASPRTWRESSLNTTSHVEINFIEKFTSGRSLRPAV
RCSMTWFLSWSPCWECARAIREFLHQHPNVSLVIYVARLYWHVDEQNRQGLRDLV
TSGVRVQIMSDSEYRHCWRNFVNFPPGQEAGWPRFPPMWTTLYA
>F7F6M6_CALJA/24-178
(SEQ ID NO: 449)
YISYDPKELCKETCLLYEIKWGMSWKIWRSSGKNTTNHVEINFIEKFTSERHFHLSVS
CSITWFLSWSPCWECSQAIREFLSQHPGVTLVIYVARLFQHMDQQNRQGLRDLVNSG
VTIQMMTVSEYYHCWRNFVNYPPGEEAHWPRHPPLWLMLY
>A0A096MWB4_PAPAN/24-177
(SEQ ID NO: 450)
DIFYDPRELRKEACLLYEIKWGMSPKIWRSSGKNTTNHVEVNFIEKLTSERRFHSSISC
SITWFLSWSPCWECSQAIREFLSQHPGVTLVIYVARLFWHTDQQNRQGLRDLVNSGV
TIQIMTASEYYHCWRNFVNYPPGEEAHWPRYPPLWMML
>G1QZV0_NOMLE/24-177
(SEQ ID NO: 451)
DVFYDPRELRKEACLLYEIKWGMSQKIWRSSGKNTTNHVEVNFIKKFTSEGRFQSSIS
CSITWFLSWSPCWECSQAIREFLSQHPGVTLVIYVARLFWHMDQQNRQGLRDLVNS
GVTIQIMRASEYYHCWRNFVNYPPGDEAHWPRYPPLWMML
>H2Q5C6_PANTR/23-177
(SEQ ID NO: 452)
FDVFYDPRELRKEACLLYEIKWGMSRKIWRSSGKNTTNHVEVNFIKKFTSERHFHPSI
SCSITWFLSWSPCWECSQAIREFLSQHPGVTLVIYVARLFWHMDQQNRQGLRDLVNS
GVTIQIMRASEYYHCWRNFVNYPPGDEAHWPQYPPLWMML
>K7G211_PELSI/189-300
(SEQ ID NO: 453)
ERSLNPLTHCSVTWFLSWSPCWKCSQSVVEFRKAYPKVNLEIYVARLFRHEEECNRQ
GLRDLVMNGVTIRVMNLSAYNYCWRTFVSHQGDDYWPWHLT
>A0A151P6M4_ALLMI/21-173
(SEQ ID NO: 454)
FQENYMPSTWPKVTHLLYEIRWGKGSKVWRNWCSNTLTQHAEVNCLENAFGKLQF
NPPVPCHITWFLSWSPCCQCCRRILQFLRAHSHITLVIKAAQLFKHMDERNRQGLRDL
VQSGVHVQVMDLPDYRYCWRTFVSHPEGEGDFWPWFF
>A0A091EQ78_CORBR/9-142
(SEQ ID NO: 455)
FQINYSPSQHRRGVYLLYEIRWRRGSIWRNWCSNTHRQHAEVNFLENCFKDRPQVP
CSITWFLSASPCGKCSKRILEFLKSRPYVTLKIYAAKLFRHHDIRNREGLCNLGMHGV
TIHIMNLEDYSYCWRNFVVY
>U3JXR8_FICAL/251-385
(SEQ ID NO: 456)
FQRNYSPSQNGRVVYLLYEIRWKGGSIWRNWCSNNPEQHAEINFLENRFNDRPQTSC
SITWFLSTSPCGKCSKRILEFLRSHPNVTLKIYTAKLFRHYEIRNRQGLRNLIMNGVAV
HIMNLEDYSYCWTNFVAHQ
>H0ZSB3_TAEGU/8-142
(SEQ ID NO: 457)
FQRNYSPRQHGRVVYLLYEIRWRRGSIWRNWCLNNHEQHAEVNFLENHENDRPQTP
CSITWFLSTSPCGKCSRRILDFLRSHPNVTLVIYAAKLFKHHDIRNRQGLRNLNMNGV
TIRIMNVEDYRYCWRNFVAY
>A0A093PWR2_9PASS/7-142
(SEQ ID NO: 458)
FKRNYLPGQHPQVVYLLYEIRWRNGSIWRNWFSNNRNQHAEVNFLENCFSDVPPAP
CSITWFLSTSPCGKCSRRILEFLRTHRNVTLEIYAAKLFRHQDIRNRQGLCNLVMNGV
TIHIMNLADYSYCWKRFVAY
>A0A091MEP8_9PASS/7-142
(SEQ ID NO: 459)
FQRNYLPDQHPQAVYLLYEFRWRRGSIWRKWCSNNRAQHAEVNFLENCFNGIPPVP
CSITWFLSTTPCGNCSRRILEFLRLHPNVTLEIYAAKLFRHTDIRNRKGLYNLAMNGVI
IRIMNLADYSYCWRNFVAY
>A0A091RF60_9GRUI/11-129
(SEQ ID NO: 460)
RNYLPGCYPKVVYLLYEIKWRRGTTWRNWCSNSRNLHAEVNFLENCFKAVPSVSCS
MTWFLSAIPCGKCSRRILEFLKVYPNVTLEIYAAKLFKHLDIRNRQGLRNLAMNGVII
RIMNL
>A0A091IIG0_CALAN/8-142
(SEQ ID NO: 461)
FKRNYQPGRRPNVVYLLYEIRWRRGTIWRNWCSNEFPQHAEDNFFQNRFNAVPSVS
CSITWFLSTTPCGRCSKRILEFLRLHPNVTLKIYAARLFRHLDNRNRQGLRKLASNGV
IIQIMGLPDYSYSWKKFVAY
>A0A091QEK6_MERNU/8-142
(SEQ ID NO: 462)
FKTNYSPDHRPRVVYLLYEIRWRRGTIWRNWCSNNIDQHAEVNFLENCFKAKPSVS
CSITWFLSTAPCAKCSRRILKFLTAHPKVTLEIYAAKLFRHLEIRNRQGLMDLAVNGV
ILRIMNLADYSYCWKQFVAY
>A0A093GVH6_PICPB/10-142
(SEQ ID NO: 463)
KLNYVPVGRPRVVYLLYEIRWSRGSIWRNWCSNSSTQHAEVNFLENCFKAMPSVSC
SITWFLSTTPCGNCSRRILEFLRAHPKVTLAIHAAKLFKHLDVRNRHGLKALATDGV
VLHIMSIADYRYCWTKFVAY
>A0A091PSV3_HALAL/7-142
(SEQ ID NO: 464)
FKRNYLPGQHPKVVYLLYEIRWSRGTIWRNWCSNNSTQHAEVNFLENCFKATPSVS
CSITWVLSTTPCGKCSRRILEFLRVHPNVTLEIYAAKLFKHLDIRNRKGLRDLAMNGV
IIRIMNLSDYSYCWKTFVAY
>A0A091M4D7_CARIC/8-142
(SEQ ID NO: 465)
FKRNYLPGQHPEVVYLLYEIKWNSGTIWRNWCSNNPTQHAEVNFLENHFNVMSSVS
CSITWGISTTPCGKCSRRILEFLTTHPNVTLEIYAAKLFKHLDIRNRQGLRNLAMNGV
VICIMNLADYSYFWKTFVAY
>A0A099ZZX4_CHAVO/10-142
(SEQ ID NO: 466)
IRNYLPDKHPNVVYLLYEIRWSRGTIWRNWCSNNSTQHAEVNFLENCFKAMPSVSC
SITWFLSTTPCGRCSRRILKFLRVHPNVTLKIHAAKLFKHLDMRNRQGLKNLAMHGV
IIRIMNLADYSYCWKTFVAH
>A0A094MFH1_ANTCR/11-142
(SEQ ID NO: 467)
RNYLPVQYPNMVYLLYEIRWSTGTIWRNWCSNNSTQHAEVNFLENRFNSRPSVSCSI
TWVLSTTPCGKCSTKILEFLRLHPNVTLKIYAAKLFKHLDIRNRQGLRNLAMNGVIIRI
MNLADYSYCWKTFVAY
>A0A093FY71_TYTAL/8-142
(SEQ ID NO: 468)
FKRNFLPGQHPKVVYLMYEIRWIRGTAWRSWCSNNSKQDAEVNLLENCFKAMPSV
FCSVTWVLFTTPCGKCFRRILEFLRVHSNVALERYAAQLFRHLDICNWQGIRSLAMN
GVIIHIMNLADYSYCWKRFVAY
>A0A091TCM6_PHALP/7-142
(SEQ ID NO: 469)
FKRNYSPGQHPKVVYLLYEIRWSRGTTWRNWCSNNSTQHAEVNFLENCFKAMPSVF
CSITWVLSTTPCGKCSRRIQEFLRVHPNVTLEIYAAKLFKHLDRRNRQGLRNLAMNG
VIIRVMNLADYRYCWKRFVAY
>A0A093CIQ8_9AVES/5-130
(SEQ ID NO: 470)
FKINNLPGQHPRVVCLLYAIRWSRSTLWKSWCSNNSTQHAEVNFLENCFKGNPSVFC
FMTWFFHTTPHGKCCRRTPEFLGVHPNVTLKIRAAKLFKHLDRYNQQGLRNVAMN
GVVIRIINL
>A0A093F3R4_GAVST/8-142
(SEQ ID NO: 471)
FKRNYLPAQHPKVVYLLYEIRWSRGTIWRNWCSNNSTQHAEVNFLENCFKAMPSVS
CSITWFLSTTPCGKCSRRILTFLREHPNVTLEIYAAKLFKHLDVRNQQGLRNLDRNGV
IIRIMNFADYSYCWKRFVAY
>A0A093LP85_FULGA/9-142
(SEQ ID NO: 472)
FKRNFLPSKYPKVVYLLYEIRWSSGTIWRSWCSNNSTQHAEVNFLENCFKAMPSVSC
SITWVLPITPCGKCSKKILEFLSVHPNVTLEIYAAKLFRHLDIRNQQGLRNLAMNGVII
RIMNLADYSYSWKRFVAY
>A0A091SSF0_9AVES/7-142
(SEQ ID NO: 473)
FKRNYLPGQHPKVVYLLYEIRWSRGTIWRSWCSNNSKQHAEVNFLENCFKARPSVS
CSITWVLSTTPCGKCSRRILEFLRVHPNVTLEIYAAKLFKHLDIRNQQGLRNLAMNGV
IIRIMNLADYSYCWKRFVAH
>A0A093JI54_EURHL/9-142
(SEQ ID NO: 474)
FKRNYMPSQYPKVVYLLYEIRWSRGTVWRNWCSNSFTQHAEVNFLE
NYFKPMPSVSCSITWVLSTTPCGKCSRRILEFLRVHPNVTLEIYAAKLFKHLDIRNRQ
GLRDLAMNGVTIRIMNLADYSFCWKRFVAY
>A0A087QNJ5_APTFO/8-142
(SEQ ID NO: 475)
FKRNYLPGQHPKVVYLLYEIRWSRGTIWRNWCSNNSTQHAEVNFLENCFKAMPSVS
CSITWVLSTTPCGKCSRRILEFLRVHPNVTLEIYAAKLFKHLDIRNRQGLRNLAMNGV
IIRIMNLADYSYGWKRFVAY
>A0A093RC01_PHACA/9-142
(SEQ ID NO: 476)
FKRNYSPCQHPKVVYLLYEIRWRRGAIWRSWCSNNSTQHAEVNFLENCFRAMPSAS
CSITWVLSTSPCGKCSRRILEFLRVHPNVTLEIYAARLFKHLDTRNRQGLRNLAMEGV
VIRIMNLADYSYWWKRFVTY
>A0A091V7F8_NIPNI/10-142
(SEQ ID NO: 477)
RSNYLPCQHPRVVYLLYEIRWSRGTIWRNWCSNNSTQHAEVNFLENCFKAMPSVPC
SITWVLSTTPCGKCSRRILEFLRVHPNVTLEIYAAKLFKHLDIRNRQGLRNLAKNGVV
IRIMKLADYSYWWKRFVAY
>A0A091XJL0_OPIHO/7-142
(SEQ ID NO: 478)
FKRNYLPGQHPKVVYILYEIRWSRGTIWRNWCTNNSTQHAEVNFLENCFKAMPSVS
CSITWVLSTTPCGKCSKRIQDFLRIYPNVTLEIHAAKLFKHLDTRNREGLRNLAKDGV
IIHIMNLADYSYWWKRFVAY
>A0A091RU17_NESNO/8-142
(SEQ ID NO: 479)
FKRNYLPYQHPKVVCLLYEIRWNRGTIWRSWCSNNSTQHAEVNFLENCFKAKPSVS
CSITWVLSTTPCGECSRRILDFLSVYPNVTLKIYAAKLFKHLDNRNRQGLWNLANNR
VIIRIMNLEDYNYYWKRFVAY
>A0A0Q3WQU9_AMAAE/56-187
(SEQ ID NO: 480)
FKRNYLPNRHPKVVCLLYEIRWSRGTIWRNWCSNSSTQHAEVNFLENCFKANPSVSC
SITWVLSTTPCGKCSRRILDFLSGYPNVTLEIYAAKLFKHLDNRNRQGLWNLANNRV
SIHIMNLAGSGKLLV
>A0A094LEL8_9AVES/8-141
(SEQ ID NO: 481)
FKGNYLPDKHPRVVYLLYEIRWSRGTIWRNWCSNSSTQHAEINFLENCFKTSKTVSC
SIIWVLSTTPCGKCSRRILEFLREHPNVTLEIHAAKLFKHLDTRNQQGLRDLAMKGVII
HIMNVADYSYWWKRFVA
>A0A087VMP5_BALRE/8-142
(SEQ ID NO: 482)
FKRNYLPGKHPRVVYLLYEIRWSRGTIWRSWCSNNATQHAEINFLETCFLARTSVSC
SITWVLSTTPCGKCSRRILEFLNAYPNVTLEIYAAKLFRHLDNRNRQGLRNLAMKGV
RIHIMNLADYSYFWKIFVAY
>A0A091GLR0_BUCRH/7-142
(SEQ ID NO: 483)
FTRNYLPNQHPRVVYLLYEIRWRRGTIWRNWCSNNSTQHAEIKFLENCFNATTSVSC
SIIWFLSTTPCGKCSTRILEFLRAHPNVTLEIYAAKLFKHHDNRNRRGLWNLAMNGV
KLHIMNPADYSYCWKMFVAY
>I3M955_ICTTR/250-417
(SEQ ID NO: 484)
FHLQFNNLHRPCRRKTYLCYQLRLGSLCDQDYFQNKDLHAEIRFIKKIRSLDLDQSH
NYEVTCYLTWSPCPDCAQELVALTRSHPHVRLTLFTSRLYFHWLWRFQEGLRLLWR
SGVQIRVMSLREFTHCWVKFVNHGGCPFEPWDGLEQRSQSIQNRLNR
>G312J2_CRIGR/230-389
(SEQ ID NO: 485)
NRHRVRYQRKTYLCYLLEQNGQQPLKGCLQNKGKHAEILFIDEMRSLELGQVQITC
YLTWSPCPNCAQELAAFKSDHPDLVLRIYTSRLYFHWRRKYQEGLCCLWRSGIQVD
VMDLPQFADCWTNFVNQSPFWPWNNLEKNSRCIQRRLQR
>F7EWS7_RAT/229-389
(SEQ ID NO: 486)
NSHRVRYRRKSYLCYQLERNGQEPLKGYLLYKGQHVEILFLEKMRSMELSQVRITC
YLTWSPCPNCARQLAAFKKDHPDLILRIYTSRLYFYWRKKFQKGLCTLWRSGIHVDV
MDLPQFADCWTNFVNQRPFRPWNELEKNSWRIQRRLRR
>ABEC3_RAT/241-405
(SEQ ID NO: 487)
FYSQFYNQRVHGVKPYLCYQLEQNGQAPLKGCLLSEGQHAEILFLDKIRSMELSQVII
TCYLTWSPCPNCAWQLAAFKRDRPDLILHIYTSRLYFHWKRPFQKGLCSLWQSGILV
DVMDLPQFTDCWTNFVNKRPFWPWKGLEIISRRTQRRLHR
>A0A091EM42_FUKDA/548-715
(SEQ ID NO: 488)
FRIQFNNAYKPHRRVTYLCYQVQKNGNLLTKGCLRTKGYHAESRFIKRICSLGLDQA
QSYQVTCFLTWSPCPRCAQELVLFKSSHPHLYLRIFTARLYFHWRKSYQEGLQRLCR
AQVPVAVMGYPEFAHCWYNFVDHPGPFEPWYKMEYYSKCIKKRFQR
>G5AYU5_HETGA/236-403
(SEQ ID NO: 489)
FRVQFNNAYKPRRRVTYLCYQLQENGDPLTKGCLRTKGYHAESRFIKRICSMDLGQ
DQSYQVTCFLTWSPCPHCAQELVSFKRAHPHLRLQIFTARLFFHWKRSYQEGLQRLC
RAQVPVAVMGHPEFAYCWDNFVDHPGPFEPWAKLEYYSSCLKRRLQQ
>T0NHJ8_CAMFR/587-753
(SEQ ID NO: 490)
FNKQFGNQPRPYRRKTYLCYQLKGNGSILAQGCVRNKQRHAEIRFIDKINFMNLNPN
QSYEIICYVTWSPCPTCAEKLVDLINDQVHLKLQIFASRLYFHWVRKYQIGLQYLWA
SQVTVAVMNRQEFKDCWEKFVDNGKDFQGWYKLEEYNRSISRRLNR
>B7T155_BOVIN/34-200
(SEQ ID NO: 491)
FKQQFGNQPRPYRRKTYLCYQLKQNDLTLDRGCFRNKQRHAEIRFIDKINSLDLNPS
QSYKIICYITWSPCPNCANELVNFITRNNHLKLEIFASRLYFHWIKSFKMGLQDLQNA
GISVAVMTHTEFEDCWEQFVDNSRPFQPWDKLEQYSASIRRRLQR
>F7IF99_CALJA/10-175
(SEQ ID NO: 492)
NIQLTNPYPKRTYLCYQLMPNGSTPTRGYFKNKNRHAEICFIDEIESMGLDKTQCYEV
TCYLTWSPCPSCAQKLVAFAKAQVHLNLRIFASRLYYHWLLSCKKGLQLLWKSQIP
VEVMGLPEFTDCWENFVVHGPPPFNPSEKLQELGSRSIKRRLDK
>A0A096NK51_PAPAN/9-179
(SEQ ID NO: 493)
FSLQFNNKRRPYPRKALLCYQLTPNGSTPTRGYLKNKKNHAEIRFINKIKSMGLDETQ
CYQVTCYLTWSPCPSCAGKLVDFLKAHRHLNLSIFASRLYYHWRPNYQEGLLLLCG
SQVPVEVMGLPEFTDCWENFVDHEPPSFNPSEKLEELDSRAIKRRLER
>A0A0D9R222_CHLSB/9-179
(SEQ ID NO: 494)
FSLQFNNKHHPYRRKALLCYQLTPNGSTPTRGQLQNKKDHAEIRFINKIKSMGLDET
QCYQVTCYLTWSPCPSCARELVDFIKAHNHLNLSIFASRLYYHWRPHYQEGLLLLCG
SRVPVEVMGLPEFTDCWENFVDHKPPSFNPSEKLDELDSQAIKRRLER
>F6SJ45_MACMU/9-179
(SEQ ID NO: 495)
FSLQFNNKRRPYPRKALLCYQLTPNGSTPTRGHLKNKKDHAEIRFINKIKSMGLDETQ
CYQVTCYLTWSPCPSCAGELVDFIKAHRHLNLRIFASRLYYHWRPNYQEGLLLLCGS
QVPVEVMGLPEFTDCWENFVDHKPPSFNPSEKLKELDSQAIKRRLER
>GIRYZ5_NOMLE/9-179
(SEQ ID NO: 496)
FRLQFNNKRRPYPRKALLCYQLTPNGSTPTRGYFKNKKRHAEIRFINKIKSMGLDETQ
CYQVTCYLTWSPCPSCAQELADFIKAHDHLNLRIFASRLYCHWCRRQQEGLRLLCGS
QVPVEVMGFSEFADCWENFVDYEPLSFNPSEMLEELDSRAIKRRLEK
>H2P4F3_PONAB/9-179
(SEQ ID NO: 497)
FSLQFNNKRRPYPRKALLCYQLTPNGSTPTRGYFKNKKCHAEIRFINEIKSMGLDETQ
CYQVTCYLTWSPCPSCVRELVAFIKAHDHLNLRIFASRLYCHWCRRQQEGLRLLCGS
QVPVEVMGSREFADCWENFVDHKPLSFNPSEMLEELDSRAIKRRLER
>H2QLQ0_PANTR/9-179
(SEQ ID NO: 498)
FRLQFNNRRRPYPRKALLCYQLTPNGSTPTRGYFENKKCHAEICFINEIKSMGLDETQ
CYQVTCYLTWSPCSSCAWKLVDFIQAHDHLNLRIFASRLYYHWCKPQQEGLRLLCG
SQVPVEVMGLPEFNDCWENFVDHEPLSFDPCKMLEELDSRAIKRRLER
>M4W6S4_HUMAN/9-179
(SEQ ID NO: 499)
FRLQFNNKRRPYPRKALLCYQLTPNGSTPTRGYFENKKCHAEICFINEIKSMGLDETQ
CYQVTCYLTWSPCSSCAWELVDFIKAHDHLNLGIFASRLYYHWCKPQQKGLRLLCG
SQVPVEVMGFPEFADCWENFVDHEPLSFNPYKMLEELDSRAIKRRLER
>F7AL68_MONDO/6-173
(SEQ ID NO: 500)
FLYHFKNVRWAGRHETYLCYVVKRDSATSFLDFGYLRNKGCHVELIFLRYISAWDL
DPSRCYRVTWFTSWSPCYDCARHVANFLRCYPNLTLRIFTARLYFCEDKKPEGLRRL
HRAGVQIAIMTFKDYFYCWNTFVENERTFKAWEGLHENSVRLSRQLRR
>G3W3P5_SARHA/11-178
(SEQ ID NO: 501)
FLYHFKNVRWAGRHETYLCFVVKRDSATSFLDFGYLRNKGCHVELLFLQYISAWDL
DPSRCYRVTWFTSWSPCYDCARHVANFLRCYPNLSLRIFTARLYFCEDKKPEGLRRL
HQAGVPIAIMTFKDYFYCWNTFVENERTFKAWEGLHENSVRLSRQLRR
>F7A0K1_HORSE/11-178
(SEQ ID NO: 502)
FLYHFKNVRWAGRHETYLCYVVKRDSATSFLDFGHLRNKGCHVELLFLRYISDWDL
DPGRCYRVTWFTSWSPCYDCARHVADFLRGYPNLSLRIFAARLYFCEDRKPEGLRRL
HRAGVQIAIMTFKDYFYCWNTFVENERTFKAWEGLHENSVRLSRQLRR
>G3SM91_LOXAF/9-176
(SEQ ID NO: 503)
FLYQFKNVRWAGRHETYLCYVVKRDSATSFLDFGHLRNKGCHVELLFLRYISDWDL
DPGRCYRVTWFTSWSPCYDCARHVTDFLRGYPNLTLRIFTARLYFCEGRKPEGLRRL
HRAGVQIAVMTFKDYFYCWNTFVANERTFEAWEGLHENSVRLTRQLRR
>L9KY18_TUPCH/51-185
(SEQ ID NO: 504)
MYPLWGRHETYLCYVVKRDSATSFLDFGHLRNKGCHVELLFLRYISDWDLDPDRCY
RVTWFTSWSPCYDCARHVADFLRGYPNLTLRIFTARLYFCEDQKPEGLRRLHRAGV
QLAIMTFK
>W5NV85_SHEEP/11-181
(SEQ ID NO: 505)
FIYQFKNVRWAGRHETYLCYVVKRDSPTSFLDFGHIRNKGCHVELLFLRYISDWDLD
PGRCYRVTWFTSWSPCYDCARHVADFLRGYPNLSLRIFTARLYFCDKERKPEGLRQL
HRAGVQIAIMTFKDYFYCWNTFVENERTFKAWEGLHENSVRLSRQLRR
>H0W743_CAVPO/11-178
(SEQ ID NO: 506)
FLYQFKNVRWAGRHETYLCYVVKRDSATSCLDFGHLRNKGCHVELLFLRYISDWDL
DPGRCYRVTWFTSWSPCYDCARHVADFLRGNPNLSLRIFTARLYFCEDRKPEGLRRL
HRAGVQIAVMTFKDYFYCWNTFVENEKTFKAWEGLHENSVRLSRQLRR
>G3QLD2_GORGO/11-168
(SEQ ID NO: 507)
FLYQFKNVRWAGRRETYLCYVVKRDSATSFLDFGYLRNKGCHVELLFLRYISDWDL
DPGRCYRVTWFTSWSPCYDCARHVADFLRGNPNLSLRIFTARLYFCEDRKPEGLRRL
HRAGVQIAIMTFKENERTFKAWEGLHENSVRLSRQLRR
>AICDA_HUMAN/11-178
(SEQ ID NO: 508)
FLYQFKNVRWAGRRETYLCYVVKRDSATSFLDFGYLRNKGCHVELLFLRYISDWDL
DPGRCYRVTWFTSWSPCYDCARHVADFLRGNPNLSLRIFTARLYFCEDRKPEGLRRL
HRAGVQIAIMTFKDYFYCWNTFVENERTFKAWEGLHENSVRLSRQLRR
>G5BPM7_HETGA/64-219
(SEQ ID NO: 509)
FLYHFKNVRWAGRHETYLCYVVKRDSATSFLDFGYLRNKGCHVELLFLRYISDWDL
DPGRCYRVTWFTSWSPCYDCARHVADFLRGNPNLSLRIFTARLTGWGPAGLMSPAR
PSDYFYCWNTFVENERTFKAWEGLHENSVRLSRRLRR
>L5KIU3_PTEAL/6-173
(SEQ ID NO: 510)
FLYHFKNVRWAGRHETYLCYVVKRDSATSFLDFGHLRNQGCHVELLFLRYISDWDL
DPGRCYRVTWFTSWSPCYDCARHVADFLRGNPNLSLRIFTARLYFCDGFKPEGLRRL
HRAGVQIAIMTFKDYFYCWNTFVENEKTFKAWEGLHENSVRLSRQLRR
>F1SLW5_PIG/11-179
(SEQ ID NO: 511)
FLYQFKNVRWAGRHETYLCYVVKRDSATSFLDFGHLRNKGCHVELLFLRYISDWDL
DPGRCYRVTWFTSWSPCYDCARHVANFLRGNPNLSLRIFTARLYFCDGYKPEGLRR
LHRAGVQIAIMTFKDYFYCWNTFVENERSFKAWEGLHENSVRLTRQLRR
>S7N9P5_MYOBR/10-177
(SEQ ID NO: 512)
FLYHFKNVRWAGRHETYLCYVVKRDSATSFLDFGHLRNKGCHVELLFLRYISDWDL
DPGRCYRVTWFTSWSPCYDCARHVADFLRGNPNLSLRIFTARLYFCEDYKPEGLRRL
HRAGAQIAIMTFKDYFYCWNTFVENERTFRAWEGLHENSVRLSRQLRR
>G1TZP8_RABIT/6-173
(SEQ ID NO: 513)
FLYHFKNVRWAGRHETYLCYVVKRDSATSFLDFGYLRNTGCHVELLFLRYISDWDL
DPGRCYRVTWFTSWSPCYDCARHVADFLRGNPNLTLRIFTARLYFCEDRKPEGLRRL
HQAGVQLGIMTFKDYFYCWNTFVENERTFKAWEGLHENSVRLSRQLRR
>G3V7Y8_RAT/11-178
(SEQ ID NO: 514)
FLYHFKNVRWAGRHETYLCYVVKRDSATSFLDFGHLRNKGCHVELLFLRYISDWDL
DPGRCYRVTWFTSWSPCYDCARHVAEFLRWNPNLSLRIFTARLYFCEDRKPEGLRRL
HRAGVQIGIMTFKDYFYCWNTFVENERTFKAWEGLHENSVRLTRQLRR
>G1KTX0_ANOCA/12-179
(SEQ ID NO: 515)
FLYHFKNLRWAGRHETYLCYVVKQNSATSCLDFGYLRNKGCHVEVLFLRYISTWDL
DPRHCYRITWFTSWSPCYDCARHVADFLSAYPNLSLRIFAARLYFCEERNPEGLRRL
HRAGAQIAIMTFKDYFYCWNTFVENKTTFKAWEGLHENSVRLARRLRR
>F7EGY6_XENTR/13-181
(SEQ ID NO: 516)
FLYHYKNLRWAGRHETYLCYIVKRYSSVSCLDFGYLRNRGCHAEMLFLRYLSVWV
GHDPHRAYRVTWFSSWSPCYDCAKRTLEFLKGHPNFSLRIFSARLYFCEERNPEGLR
KLQKAGVRLAVMSYKDYFYCWNTFVESERRFEAWDGLHENSVRLARKLRR
>H3ALQ6_LATCH/20-149
(SEQ ID NO: 517)
FLYHYKNVRWAGRHETYLCYIVKRYNPASYLDFGFLRNKGCHVEMLFLRFLTGWNI
DPTLPYSVTWFTSWSPCYDCSQHVTHFLRVYPNLRLRIFTARLYFCEENNPEGLRNL
HMAGVQLGVMT
>A0A0P7ULF7_9TELE/8-184
(SEQ ID NO: 518)
FIYHYKNVRWAGRHETYLCFVVKRDGPDTLFDFGHLRNRGCHVELVFLRHLGALCP
GLSYSVTWFCSWSPCYNCSRRLAHFLTRTPNLKLRIFCSRLYFCDVEDSSEGLRLLKR
AGVQLSVMTYKDYFYCWQTFVARERGFKAWEGLHQNSVRLARKLNR
>B3DGZ0_DANRE/15-191
(SEQ ID NO: 519)
FIFHYKNVRWAGRHETYLCFVVKRIGPDSLFDFGHLRNRGCHVELLFLRHLGALCPG
LCYSVTWFCSWSPCSKCAQQLAHFLSQTPNLRLRIFVSRLYFCDEEDSREGLRHLKR
AGVQISVMTYKDFFYCWQTFVARERSFKAWDGLHENSVRLVRKLNQ
>I3K4U3_ORENI/15-191
(SEQ ID NO: 520)
FLYHYKNVRWAGRNETYLCFVVKRVGPDSLFDFGHLRNRGCHVELLFLRQLGTLCP
GLSYSITWFCSWSPCANCSSRLAQFLKQTPNLRLRIFVSRLYFCDMEDSREGLRLLKK
VGVHITVMSYKDFFYCWENFVAQQSKFKAWEGLHQNTVRLARKLNR
>A0A0F8AS01_LARCR/15-192
(SEQ ID NO: 521)
FIFHYKNVRWAGRHETYLCFVVKRVGPDTLFDFGHLRNRGCHVELLFLRYLGALCP
GLSYSVTWFCSWSPCADCSFRLSQFLNRTPNLRLRIFVSRLYFCDMENSREGLRMLK
NAGAHITVMSYKDFFYCWQTFVARESNFKAWDELHRNSVRLSRKLHR
>H2SYA6_TAKRU/15-192
(SEQ ID NO: 522)
FIYHYKNVRWAGRHETYLCFVVKRVGPDTLFDFGHLRNRGCHVELLFLRYLGALCP
GLSYSVTWFCSWSPCVNCSIQLCQFLNNTPNLRLRIFVSRLYFCDLEDSREGLRMLTK
AGVRISVMSYKDYFYCWQKFVDCKSNFKAWEELHQNSVRLTRKLNR
>G3P8J1_GASAC/15-192
(SEQ ID NO: 523)
FIYHYTNMRWAGRHETYLCFVVKRVGPDSLFDFGHLRNRGCHVELLFLRHLGALCP
GFSYSITWFCSWSPCVNCSISLSQFLSRTPNLRLRIFVSRLYFCDMENSRDGLRMLKK
AGVQVTVMSYKDFFYCWQTFVDRQSQFKAWKELHQNSVRLSRKLKR
>W5L8S5_ASTMX/15-190
(SEQ ID NO: 524)
FIYHYKNVRWAGRHETYLCFVVKRIGPNSLFDFGHLRNRGCHVELSEIQSFSAFCPAL
VQNDSKSCRISTDSESLHQIETSLDKTPKTGIKVFLSLSLFVTLANHSHYPQMASDLCL
CVPIFLFTDFFYCWQTFVARESRFKAWDGLHQNSVRLSRKLKR
>H2M862_ORYLA/11-188
(SEQ ID NO: 525)
FIYHYKNMRWAGRHETYLCFVVKRVGPESLFDFGHLRNRGCHVELLFLRHLSALCP
GLSYSITWFCSWSPCANCSFRLAQFLSQTPNLRLRIFVSRLYFCDLEDSREGLRMLKK
VGVHITVMSYKDYFYCWQTFVARQSKFKPWDGLHQNSVRLSRKLNR
>A0A096M3S2_POEFO/26-203
(SEQ ID NO: 526)
FIYHYKNLRWAGRCETYLCFVVKKVGPDSLFDFGHLRNRNCHVELLFLRHLGALCP
GLSYSVTWFCSWSPCANCSIRLAQFLHQTPNLRLRIFVSRLYFCDLEDSREGLRILKK
AGVHITVMSYKDYFYCWQTFVAKQSKFKPWDGLHQNYIRLSRKLNR
>A0A087XZI4_POEFO/119-308
(SEQ ID NO: 527)
FIYHYKNLRWAGRCETYLCFVVKKVGRNRLFDLNVTMNNKPLHLQLLFLRHLGALC
PGLSYSVTWFCSWSPCANCSIRLAQFLHQTPNLRLRIFVSRLYFCDLEDSREGLRILKK
AGVHITVMSYKDYFYCWQTFVAKQSKFKPWDGLHQNYIRLSRKLNR
>K7G3N4_PELSI/9-176
(SEQ ID NO: 528)
FLYNFKNLRWAGRHETYLCYVVKRDSATSFLDFGYLRNKGCHVEMLFLRYISAWDL
DPGRCYRVTWFTSWSPCYDCARHVADFLRAYPNLTLRIFAARLYFCEDRNPEGLRR
LHRAGVQIAIMTFKDYFYCWNTFVENERTFKAWEGLHENSVRLSRRLRR
>A0A151P6G3_ALLMI/11-178
(SEQ ID NO: 529)
FLYNFKNLRWAGRHETYMCYVVKRDSATSCLDFGYLRNKGCHVEVLFLRYISAWD
LDPGRCYRVTWFTSWSPCYDCARHVADFLRAYPNLTLRIFVARLYFCEGRNPEGLR
RLHRAGAHIAIMTFKDYFYCWNTFVENERTFKAWEGLHENSVHLTRKLRR
>A0A091GPU6_BUCRH/9-176
(SEQ ID NO: 530)
FLYNFKNLRWAGRRETYLCYVVKRDSATSCMDFGYLRNKGCHVELLFLRYISAWD
LDPGRCYRITWFTSWSPCYDCARHVADFLRAYPNLTLRIFAARLYFCEDRKPEGLRR
LHRAGAQIAIMTFKDYFYCWNTFVENEKTFEAWEGLHENSVHLTRKLRR
>A0A093H512_PICPB/9-176
(SEQ ID NO: 531)
FLYNFKNLRWAGRRETYLCYVVKRDSATSCLDFGYLRNKGCHVEVLFLRYISAWDL
DPGRCYRITWFTSWSPCYDCARHVADFLRTYPNLTLRIFAARLYFCEDRKPEGLRRL
HKAGAQIAIMTFKDYFYCWNTFVENEKTFKAWEGLHENSVHLSRKLRR
>A0A099YYC6_TINGU/9-176
(SEQ ID NO: 532)
FLYNFKNMRWAGRRETYLCYVVKRNSATSCLDFGYLRNQGCHVEVLFLRYISAWD
LDPGRCYRITWFTSWSPCYDCARHVADFLRAYPNLSLRIFTARLYFCEDRKPEGLRR
LHRAGAQIAIMTFKDYFYCWNTFVENEKTFKAWEGLHENSVRLSRQLRR
>A0A091RVC9_NESNO/9-176
(SEQ ID NO: 533)
FLYNFKNLRWAGRRETYLCYVVKRDSATSCLDFGYLRNQGCHVEVLFLRYISAWDL
DPGRCYRITWFTSWSPCYDCARHVANFLRAYPNLTLRIFTARLYFCEDRKPEGLRRL
HRAGAQIAIMTFKDYFYCWNTFVENEKTFKAWEGLHENSVHLSRKLRR
>A0A091MHE0_9PASS/9-176
(SEQ ID NO: 534)
FLYNFKNLRWAGRRETYLCYVVKRDSATSYLDFGYLRNQGCHVEVLFLRYISAWDL
DPGRCYRITWFTSWSPCYDCARHVANFLHSYPNLTLRIFTARLYFCEDRKPEGLRRL
HKAGAQIAIMTFKDYFYCWNTFVENEQTFKGWEGLRENSVHLSRKLRR
>A0A093PQH0_9PASS/9-176
(SEQ ID NO: 535)
FLYNFKNLRWAGRRETYLCYVVKRDSATSCLDFGYLRNQGCHVEVLFLRYIAAWD
LDPGRCYRITWFTSWSPCYDCAQHVANFLRSYPNLTLRIFTARLYFCEDRKPEGLRR
LHKAGAQIAIMTFKDFFYCWNTFVENEQTFKGWEGLHENSVHLSRKLRR
>A0A091EUR4_CORBR/9-176
(SEQ ID NO: 536)
FLYNFRNLRKAGRRETYLCYVVKRDSATSCLDFGYLRNQGCHVEVLFLRYIAAWDL
DPGRCYRITWFTSWSPCYDCAQHIANFLRSYPNLTLRIFMARLYFCEDRKPEGLRRL
HKAGAQIAIMTFKDYFYCWNTFVENEQTFKGWEGLHENSVHLARKLRR
>T0NHJ8_CAMFR/146-317
(SEQ ID NO: 537)
FTHNFSNHKRTHKTYLCYEVEIHGDSGIPDKGFLCNKCHVELYLLGRIRSWKLDRKL
HCRLTCFISWTPCGTCARELAEFLKENSHVSLCIFASRIYSLNDYEAGLRTLQEAGAQI
AIMTFKEFRHCWETFVDHGRPFQPWDELDINSQGLSKELQA
>H9GWR3_CANLF/15-197
(SEQ ID NO: 538)
FTQNFRNDNPSKTYLCYQVELDGSSGVLDKGSAFPGGQHAEWFLLEHIRSRNLDQK
LSYKVTCFLSWTPCEKCAEEIIRFLAKNRHVSLSILASRIYTMGPYVKGLRELYDAGV
HISIMTFRDFEYCWQTFVDHDSPFQPWADLDRRSQQLSQQLRA
>H0XN38_OTOGA/18-197
(SEQ ID NO: 539)
FTSNFTNNPAIGRRQTYLCHEVQLDGDSWVLDRGFLQSQPLHAEFCFLDRVGSWQL
NPNWHYRVTCFISWSPCFSCAQKVAMFLRRNSHVKLRILAARIYDYHPGYEEGLKA
LQGTGAQVAIMTHAEFEHCWDTFVDHGRPFQPWEGLDKNSQALSRRLQD
>G3SVX3_LOXAF/11-178
(SEQ ID NO: 540)
FRFNFINDASVGQKQTYLCYEVELDGNSWVLDRGFLLNQRRHAELCFLDRVSSWHL
DPTKHYKFTWFLSWSPCRNCAQEVVAFLGGNSHVSLSIFAPRIYDYYSGYEEGLRSL
QGAGAHVSIMTSTEFEHCWRTFVDNGCPFVPWNRLGENSQTISRRLQS
>G3TLG1_LOXAF/31-201
(SEQ ID NO: 541)
FQLNFINDLSVGQKQAYLCYEVELDCNSRVLNRGFLCNQGCHAELCFLDQVPSWQL
DLVLCYRVTWFISWSPGPDCAQEVAAFLRGNSHVSLSIFASCIYDSVEESEGGWKGK
KDLGASKKKFGHCWKTIVDNGRFFEPWNRLDESFDFLDLT
>G1TLT9_RABIT/18-182
(SEQ ID NO: 542)
FMDHFANEDGGGLNETYLCYEVQLDGSSQGFLRNKRRHAELCFLDLVPAWRLDPA
QHYRVTWFISWSPCFLCAQAVAEFLRRNAHVSLRIFAARIYTWRTDYKAGLQDLQR
AGAQIAIMTPAEIQFCWNTFVDNSNPFHSHLGLGHAKPA
>S7N2R7_MYOBR/2-126
(SEQ ID NO: 543)
CFDGNNRRPRAELCFLALFQSWHLNEGKQYRLTWYSSWSPYPDCVPKLVEFLGDNS
NVSLRIFAAGIHSIFTGYKRELRNLRDAGAQLAIMTLEELRWELGGEMATWRWQNE
N
>L7N100_MYOLU/15-180
(SEQ ID NO: 544)
FKENFANTWENETELCYEVEVEGDTWAVEQGFLCNQPCHAELCFLCLVRSWHVDE
GKQYRLTWHISWSPCPNCAQKLVKFLHDNSHVSLRIFAAGIQTTFSGHEDWLRKLRD
SGAQLAIMTLKELQHCWDTFVDNGQPFEPWPNLVEHIQTESQKLKD
>G1Q326_MYOLU/19-187
(SEQ ID NO: 545)
EWNFGITWAKETYLCYEVEVEGDAWVAKEGFLRNELRHAELCFLRGVSDWDLDEG
KQYRLTYMSWSPCPNCAPKLVEFLDENSHVTLRIFPARIHTKSRGYQDGLRNLRDAG
AQLAIMTLKEHQHCWDTFVDNGQPFRPWPNLVEHIETKSQELKD
>G1PQB2_MYOLU/4-177
(SEQ ID NO: 546)
FKDNFGHNWEKTYLWYEVEFEGDAWAVEQGFLRNQLRHAELCFLHGVRSWHLDE
GKQYRLTWHISWSPCPDCASKLVEFLGENSHVSLRIFAARIHTKYRGYEDGLRQLQD
AVDHLTIMTLKELQHCWVTFVDNGQPFEPEIELLENIGAQCQKLES
>L5M566_MYODS/4-175
(SEQ ID NO: 547)
FKDNFGITWANETYLCYEVEVEGDAWAVEQGFLRNQRSHAELCFLDRVPSWHLDE
GKQYRLTCYISWSPCPDCAQKLVEFMGENSHVSLRIFAARIYTKLDGHEDGLRKLQD
AGAQLAIMTLKEYEHCWDTFVDSGQLFRARDELEVHIGAQCQRLEN
>G1QB54_MYOLU/17-183
(SEQ ID NO: 548)
FKRNFRNKCKNQTYLCYEVEVEGNAWAVEQGFLRNQRRHAELCFLDRVPCWNLDG
LKKYRLTCYISWSPCPDCALELVQFLGLKSNVSLRMCTAGIRTTFPGHEDGLRNLRD
AGAQLTIMTRDEYEHCWDTFVDNGQPFRARDELEGHDYFLL
>GINTH0_MYOLU/7-175
(SEQ ID NO: 549)
EVNFGHDWEKKTYLCYEVEVEGDAWVGKQGFLCNQPGHAELCFLDRVRSWHLDG
GKQYRLTCYMSWTPSPDCALELVQFLSENSHVSLRIFAAGIRTRFHGHEDGLRQLRD
AGAQLAIMTLHELQHCWDTFVDNGQPFRARDELEVHIGAQCQKLKS
>L7N100_MYOLU/194-364
(SEQ ID NO: 550)
KRRNFRNKCENQTYLCYEVEVKDGKWTVEQGFLRNQPGHAELCFLDRVRSWHLDE
GRQYRLTCYISWTPGPDCAQKLVEFLGENRHVSLRIFAAGIHTKYRGHEDGLRQLW
DAGAQIAIMTLNELQHCWETFVDNGQPFEPWPIQVEHIQTESQKLKD
>G1QB54_MYOLU/248-406
(SEQ ID NO: 551)
AEKLFFKKLLQQVYYCFGLKIEGRDQPRHAELCFQDRVRSWHLDEGKQYRLTCYIS
WSPCPDCAQKLVEFLGENSHVRLRIFAARIYKKRDRYKHWLRQLRDAGAQLTIMTL
NELQHCWVTFVDNGQRFEP
>G1PBV8_MYOLU/144-276
(SEQ ID NO: 552)
FRVNFSYYRERKTYLCYEVEVEGDAWVVKQDFLRNQPRHAELCFLDGVRSWHLDE
GKQYRLTCYISWSPCPVCAQELVEFLGENRHLRLRIFAARIYSIVSGYEDGLRQLWDA
GAPLAIM
>G1Q0Q6_MYOLU/140-315
(SEQ ID NO: 553)
FKDNFSHRRARRTYLCYQVEVEGDAWAVEYGFLCNQLRHAELCFLDRVPFWNLEE
GRQYRLTCYISWSPCPDCAQRLVEFLGNNNHMRLRIFAARIYTFVSGHEDGLRQLW
DAGAQLTIMTRNDLQHCWDTFVDNGDPFEPCPIQVEHIGTESQELEN
>G1Q0G3_MYOLU/13-186
(SEQ ID NO: 554)
FKENFSHRRARKTYLCYEVEVEGNTWAVEQGFLHNQLRHAELCFLDRVRFWNLEE
GRQYRLTCYISWSPCPDCAQKLVEFLGQNSHVSLCIFAARIYTIVSGYKDGLCQLRDA
GAQLTIMTLNDLQHCWENLVDNGEPFEPCPTLVEHIETKSQELKD
>A0A096NK44_PAPAN/18-195
(SEQ ID NO: 555)
FTFNFNNDLSVGRHQTYLCYEVERDNGTWVMDRGFLHNKGCHAELCFLGEVPSWQ
LDPAQTYRVTWFISWSPCLRRGCAEQVRAFLQENTHVRLHIFAARIYDFDFLYQEAL
RTLRDAGAQVSIMTYEEFKHCWDTFVDHGRPFQRWDGLDEHSQDLSGRLRA
>A0A0D9R289_CHLSB/198-375
(SEQ ID NO: 556)
FTINFNNDLSVGRRQTYLCYEVERDNGTWVMDWGFLCNQSCHVELCFLSQVSSWQ
LDPAQTYRVTWFISWSPCFSGGCAEQVRAFLQENTHVRLCIFAARIYNYDPLYQEAL
RMLRDAGAQVSIMTYEEFEYCWDTFVDRGCPFQPWDGLDEHSQALSGRLRA
>F7FXK1_MACMU/18-195
(SEQ ID NO: 557)
FTSNFNNDVSVGRHQTYLCYEVERDNGTWVMDWGFLCNQGCHAELCFLGWVPSW
QLDPAQTYRVTWFISWSPCFSWGCAEQVRAFLQENTHVRLHIFAARIYDYDPLYQEA
LRTLRDAGAQVSIMTYDEFEYCWDTFVDCGCPFQPWDGLDEHSQALSERLRA
>GIRYY7_NOMLE/198-375
(SEQ ID NO: 558)
FTFNFNNDPLVGRHQTYLCYEVERDNGTWVMDRGFLHNQGRHTELCFLGLIPYWQL
DLAQTYRVTWFISWSPCFSWGCAEQVRAFLQENTHMRLRIFAARIYDYDPLYKEAL
QMLRGAGAQVSIMTYDEFEHCWDTFVDHGRPFQPWDGLEEHSQALSGRLQA
>H2P4E7_PONAB/198-375
(SEQ ID NO: 559)
FTFNFNNDPFVRRHQTYLCYEVEHDNGTWVMDRGSLHNQGRHAELRFLGLLPYWQ
LDPAQIYRVTWFISWSPCFSWGCARQVRAFLQENTHVRLRIFAARIYDYDPLYKEAL
QMLRDAGAQVSIMTYDEFEYCWNTFVDHGCPFQPWDGLEEHSQALSGKLQA
>GIRYY4_NOMLE/16-192
(SEQ ID NO: 560)
FTSNFNNGRWHKTYLCYEVERDNGTWVMDRGFLHNQGRHAELCFLDLVPSLQLDP
AQTYRVTWFISWSPCFSWGCAEQVRAFLQENTHVRLRLFAARIYDYDPLYKEALQM
LRGAGAQVSIMTYHEFKHCWDTFVDHGRPFQPWDGLEEHSQALSGRLQA
>H2QLP4_PANTR/16-192
(SEQ ID NO: 561)
FTSNFNNGGRRKTYLCYEVERDNGTSVMDRGFLHNQGRHAELRFLDLVPSLQLDPA
QIYRVTWFISWSPCFSWGCAGQVRAFLQENTHVRLRIFAARIYDYDPLYKEALQMLR
DAGAQVSIMTYDEFKHCWDTFVDHGCPFQPWDGLEEHSQALSGRLRA
>G3S2J9_GORGO/18-195
(SEQ ID NO: 562)
FTSNFNNDLLVRRHQTYLCYEVERDNGTWVMDRGFLHNQGRHAELRFLDLVPSLQL
DPAQIYRVTWFISWSPCFSWGCAGQVCEFLQENTHMRLRIFAARIYDYDPLYKKALQ
MLRDAGAQVSIMTYDEFKHCWDTFVYRGCPFQPWDGLEEHSQALSGRLQA
>H2QLP5_PANTR/198-375
(SEQ ID NO: 563)
FTFNFNNDPLVRRHQTYLCYEVERDNGTWVMDMGFLCNEGRHAELRFLDLVPSLQ
LDPAQIYRVTWFISWSPCFSWGCAGQVRAFLQENTHVRLRIFAARIYDYDPLYKEAL
QMLRDAGAQVSIMTYDEFEYCWDTFVYRGCPFQPWDGLEEHSQALSGRLRA
>G3QV16_GORGO/198-375
(SEQ ID NO: 564)
FTFNFNNDPLVRRHQTYLCYEVERDNGTWVMDMGFLCNEGRHAELRFLDLVPSLQ
LDPAQIYRVTWFISWSPCFSWGCAGQVCEFLQENTHMRLRIFAARIYDYDPLYKKAL
QMLRDAGAQVSIMTYDEFKHCWDTFVYRGCPFQPWDGLEEHSQALSGRLQA
>A0A096N7U5_PAPAN/178-346
(SEQ ID NO: 565)
FTYNFTNDPSVGQHQTYLCYKVECDNDTWVLDKGILPNQHAEQYFLYLISFWKLDQ
AQCYRVTWFISWSTCFSCAQQVATFHWENRCVSLHIFIARIYNYLPGYEGLCMLQRA
GTQISIMTSKFRHCWVTFVDHGHPFQPWDGLDEHSQALSGRLQA
>F6QUT3_MACMU/171-340
(SEQ ID NO: 566)
FTYNFTNDPSVGQHQTYLCYKVECDNDTWVLDKGILPNQHAEQYFLYLISFWKLDR
AQCYRVTWFISWSTCFSCAQQVTTFHWENRCVSLHIFVACIYNYLPGYEGLCMLQR
AGTQISIMTSGFRHCWVTFVDHGHPFQPWDGLDEHSQALSGRLQA
>H9KW44_CALJA/143-282
(SEQ ID NO: 567)
FTYNFTNDPSVGRHQTYLCYEVEHHNGTWVLHRGFILNEGRHAELCLLDLILFWKL
DLAQRYRVTCFISWSPCFCCAEKVAEFLQENPHVNLRIFAARIYGYQRGYKKGLRRL
NRAGAPISMMKYS
>F7CUA6_CALJA/202-377
(SEQ ID NO: 568)
FTYNFTNDPSVGQHQTYLCYEVEHHNGTWVLHRGFILNQGRHAELCLLDLISFWKL
DLAQYYTVTCFISWSPCFSCAEKVAEFLQENPHVNLHIFAAHIYGYQRGYIKGLCRLN
RAGAPISMMKYSEFSYCWDTFVDHEHPFQPWEGLDEYTQALSGKLQA
>A0A0D9R229_CHLSB/213-376
(SEQ ID NO: 569)
FTSNFNNKPWVGQRETYLCYKVERHNDTWVLNRGFLRNQGRHAELCFLDLIPFWKL
DDQQYRVTCFTSWSPCFSCAQKMAKFISKNKHVSLCIFAARIYDDQGRCQEGLRTLH
RDGAKIAVMNYSEFEYCWDTFVDRGXXXXLGVKPDC
>ABC3G_PAPAN/202-376
(SEQ ID NO: 570)
FTSNFYNKPWVGQHETYLCYKVERHNGTWVLNRGFLRNQGRHAELCFLDLIPFWKL
DGQQYRVTCFTSWSPCFSCAQEMAKFISNNEHVSLCIFAARIYDDQGRCQEGLRTLH
RDGAKIAMMNYSEFEYCWDTFVDRGRPFQPWDGLDEHSQDLSGRLRA
>ABC3G_MACMU/195-369
(SEQ ID NO: 571)
FTSNFNNKPWVGQHETYLCYKVERHNDTWVLNRGFLRNQGRHAELCFLDLIPFWKL
DGQQYRVTCFTSWSPCFSCAQEMAKFISNNEHVSLCIFAARIYDDQGRYQEGLRALH
RDGAKIAMMNYSEFEYCWDTFVDRGRPFQPWDGLDEHSQALSGRLRA
>H2P4E9_PONAB/216-391
(SEQ ID NO: 572)
FTSNFNNEPCVGRHETYLCYKVERHNDTWVLNRGFLCNQGRHAELCFLDVIPFWKL
DGKQRYRVTCFTSWSPCFRCAQEMAKFISNNQHVSLCIFAARIYDDQGRCKEGLRTL
DEAEAKISIMTYSEFQHCWDTFVDHGRPFQPWDGLEEHSEAWSGKLQA
>ABC3G_GORGO/202-377
(SEQ ID NO: 573)
FTSNFNNEHWVGRHETYLCYEVERHNDTWVLNRGFLCNQGRHAELCFLDVIPFWKL
DLHQDYRVTCFTSWSPCFSCAQEMAKFISNKKHVSLCIFAARIYDDQGRCQEGLRTL
AEAGAKISIMTYSEFKHCWDTFVYHGCPFQPWDGLEEHSQALSGRLQA
>G3SFB6_GORGO/202-375
(SEQ ID NO: 574)
FTSNFNNEHWVGRHETYLCYEVERHNDTWVLNRGFLCNQGRHAELCFLDVIPFWKL
DLHQDYRVTCFTSWSPCFSCAQEMAKFISNKKHVSLCIFAARIYDDQGRCQEGLRTL
AEAGAKISIMTYSEFKHCWDTFVDHGCPFQPWDGLEEHSQSLGDRA
>L9KAV2_TUPCH/18-186
(SEQ ID NO: 575)
FNFHYKNLQRAGRRKTYLCYLLEEEREGAIVLDVGVLHNQEHAERRFLSSPDILQLL
DGGRRYRVTWYLSWSPCSQCARAVAGFLAQHGNVSLRIFVARLYNHEDPENRQGL
RTLNSTGTPIRVMTNREFALCWERFVRHQGAAFEPWAGLRENADLLLGQLED
>Q6DIS6_XENTR/55-231
(SEQ ID NO: 576)
FMFQFKNVEYSGRNKTILCYTVERPEGQVFHGYLEDESAHAEDAFFTSVLPQLTSGS
VTVTCYVSSSPCVNCAASVAQCLRRNKTVRIQLAVARLFQWEEPEIRRALKGLRSAG
CQVRMMRGADYVYVWKNFVEPDHQDFIPWEDLEENARYYEEKLEE
>A0A0P7WIY8_9TELE/108-272
(SEQ ID NO: 577)
FKFQFKNVEYSGRNKTFLCFLVDVQGSGGEGLRGYLEDEGAHAEEAFFQQVLPQDS
ALHYMVTWYVSSSPCAACTAKLVEILKARKTMRLTIFSARLFMWEEPEIQVGLKAL
AAAGCKLRMMKPTDFVYIWDTFVENDQTFTPWEDCQENYEYYQEKLAD
>FIRAQ7_DANRE/104-270
(SEQ ID NO: 578)
FKFQFKNVEYSGRNKTFLCYQVDIQGGETDGVRGYLEDEGSHAEEAFFQQVLAYDK
SLRYTVTWYTSSSPCVACAAKLVEILKARKALRLNIHCSRLFEWEEPEIQAGLQALVR
AGCKLRMMRPVDFVYVWSTFVENEDNFTPWEDCQDNFEYYDERLQD
>H2LVW2_ORYLA/81-243
(SEQ ID NO: 579)
FKFQFRNVEYSGRNKTLLCFRVDTAGGSTEPLRGYMEDETAHAEEAFFQQVLPNSSQ
EYDVTWYVSSSPCVACAAKLTSILQQRKKLRLSVFCSRLFDWEEPEIVQGLKALVQA
GCKLQMMKPADFQHVWETYVEKDQSFTLWEDCKENYEYYLEKLAD
>A0A087X5U3_POEFO/101-263
(SEQ ID NO: 580)
FKFQFRNVEYSGRNKTLLCFRVDTAGGNAEPLKGYMEDETAHAEEAFFQQVLPNSS
TEYDVTWYVSSSPCVACAAKLANILQQRKKLRLSIFCSRLFEWEEPEIVEGLKALARA
GCKLRMMKPIDFQHVWEMYVEQGESFTPWEDCQENYEYYVERLTD
>G3P582_GASAC/83-245
(SEQ ID NO: 581)
FKFQFRNVEYSGRNKTLLCFRVDTPGGSTEPLKGYMEDETAHAEEAFFQQVLPNTSQ
EYEVTWYLSSSPCVACAAKLAHILQQRKKVRLRMFCSRLFEWEEPEIVEGLRALVSA
GCKLRMMKPSDFVHVWETYVEKDQNFEAWEDCQENYDYYVEKLTD
>H2U6E4_TAKRU/97-259
(SEQ ID NO: 582)
FKFQFRNVEYSGRNKTLLCFRVDSPGGSTEPLKGYMEDETAHAEEAFFQQVLPNPSL
EYDITWYVSSSPCISCANTLAKMLQQRKKVRLCVFCSRLFEWDQPEVVEAIRTLVRA
GCKLRMMKPSDFQHVWETYVEKGESFTPWEDCQDNYNYYKEILAD
>A0A0P7V3P5_9TELE/68-234
(SEQ ID NO: 583)
FKFQFKNVEYSGRNKTLLCFLVDSQKGEDELQRGYLEDEGAHAEEAFFQQVLPEDP
GRRYQVTWYVSSSPCAACAAKLAEILRARPTLRLTIFSARLFMWEEPEIQEGLKALA
AAGCKLRVMKPVDFSYTWDTFVENDQTFSPWEDCQENYEYYQEKLAG
>W5KNC9_ASTMX/26-194
(SEQ ID NO: 584)
FRFQFKNVEYCGRNKTLLCYLVDQSSGADGLLRGFLEDEGLHAELAFFQLILPQDPS
VNYTVTWYVSSSPCAHCCSKLLEILQERKTLRLNIFSARLLEAEEEEGQAGMKALAG
AGCKLRVMKPLDFSYSWDTFVEHDERFTPWEDCQENYEYHHEKLAQ
>H2M4W2_ORYLA/70-234
(SEQ ID NO: 585)
FKFQFKNVEYSGRNKTLLCYLVDTGGAGDGLLRGYLEDEGIHAEEAFFTHCLPNDPN
VQYTITWYVSSSPCHACSLKLAEVLKARKNVKLSIFSARLFEWEAEEVQAGLKALHS
AGCKLRVMKPLDFSYTWDTFVETEQPLNLWEDCKDNYEYYHEKLDQ
>H2TE47_TAKRU/80-244
(SEQ ID NO: 586)
FQFQFKNVEYSGRNKTFLCYLVDTGKADEGLQRGYLEDEGSHAEEAFFTQCLPHDP
ALKYSVTWYMSSSPCCACAAKMAEALKARRNIKLSIFAARLFEWEEAEIQAGLKAL
HIAGCKIRVMKPLDFSYTWDTFVENDQPLNLWADCKENYEYYHERLAD
>I3JAF9_ORENI/80-244
(SEQ ID NO: 587)
FKFQFKNVEYSGRNKTLLCYLVDKGNTSDGLLRGYLEDEGTHAEEAFFTQCLPHNP
ALKYTVTWYVSSSPCSACAAKIAELLKDQKNLKLNIFAARLFEWEEGEIQAGLRTLH
AAGCKLRVMKPLDFSYTWDTFVENDQSLNLWEDCKENYEYYHEKLSD
>A0A0F8BFJ4_LARCR/500-664
(SEQ ID NO: 588)
FKFQFKNVEYSGRNKTFLCYLVDKGNTADGLLRGYLEDEGSHAEEVFFIQCLPHDPT
LKYTVTWYVSSSPCSACAAKIAEVLKARKNVKLSIFAARLFECEEVEIQAGLKALHA
AGCKLRVMKPLDFSYTWDTFVENEQPLNLWEDCKENYEYYHEKLAD
>G3PCV8_GASAC/21-185
(SEQ ID NO: 589)
FKFQFKNVEYSGRNKTFLCYLVDKGNTDDGLLRGYLEDEGSHAEEAFFIQCIPNDPS
VRYTVTWYVSSSPCAACAAKIAEVLKARKNIKLSIFAARLFECEETDIQAGLKAMHT
AGCKLRVMKPLDFSYTWDTFVENEEPLNLWEDCKENYEYYQEKLAD
>A0A151P4M1_ALLMI/14-181
(SEQ ID NO: 590)
FKFQFKNVEYSGRNKTFLCYIVETQGKESGTIRGYLEDEAAHAEDAFFNTILPEESSLR
YNVTWYVSSSPCVACAERIAEILKKNKNLRLAILVSRLFMWEEPEMQAVLKKMKAA
GCKLRIMKPQDFEYVWQNFVEQESKDFVPWEDIQENFLYYEEKLAE
>K7G457_PELSI/58-225
(SEQ ID NO: 591)
FKFQFRNVEYSGRNKTFLCYIVETQGKESGSFRGYLEDEAAHAEGAFFNNILPTEPSL
RYNVTWYVSSSPCVPCADQIAQILQKNKNLRLTILVSRLFMWEEPEMQAALKKLKT
AGCKLKIMKPQDFEYIWQNFVEQEAKAFEPWEDIQENFLYYEEKLAE
>M7B9Y3_CHEMY/50-217
(SEQ ID NO: 592)
FKFQFRNVEYSGRNKTFLCYVVEIQGKEPGSFQGYLEDEAAHAEDAFFNNILPTDPN
LRYNVTWYVSSSPCVPCADRIAQVLQKNKNLRLSILVSRLFMWEEPEMQAALKKLK
ASGCKLKIMKPQDFEYIWQNFAEQEAKAFEPWEDIQENFLYYEEKLSE
>V8P1W3_OPHHA/51-183
(SEQ ID NO: 593)
FNFQFKNVEYSGRNKTFLCYIIEIQGKESKMLRGYLEDEAAHAEDAFFNTILPKESGM
CYNVTWYSSCSPCIGCADRIAKALQKNKNLHLSIAVGRLFMWEEPDMQAALKKMK
TAGCKLRIMKPQDFEY
>G1KSP1_ANOCA/17-185
(SEQ ID NO: 594)
FKFQFKNVEYSGRNKTFLCYIIEMQGKESKTLRGYLEDEAAHAEEAFFNTILPTEPGF
RYEVTWYVSSSPCVSCAERIVKALKKNKNLRLSIAVGRLFMWEEPDIQAALKQMKA
AGCKLRIMKPQDFEYVWKNFVEQEPKAFAPWDDIQENFQYYDEKLAE
>R7VNA1_COLLI/52-220
(SEQ ID NO: 595)
FKFQFRNVEYSGRNKTFLCYVVETQGRESVTSRGYLEDEAAHAEMAFFNTILPKESS
ARYNVTWYVSSSPCVTCADRITETLKKNKNLRLTIMVGRLFMWEEPEMQAALKNM
RAAGCKLRIMKPQDFEYVWQNFVEQEAKAFAPWEDIQENFQYYEEKLAE
>H1A4Q1_TAEGU/51-219
(SEQ ID NO: 596)
FKFQFRNVEYSGRNKTFLCYVVETQGKEPVTSRGYLEDEAAHAEMAFFNTILPTQAG
ARHDVTWYVSSSPCVTCAQRICEALRKNKGLRLTIMVGRLFMWEEPEMQAALRSM
KEAGCKLRIMKPQDFEYVWKNFVEQEAKSFVPWEDIQENFQYYEEKLAE
>R0L7B9_ANAPL/57-225
(SEQ ID NO: 597)
FKFQFRNVEYSGRNKTFLCYVIETQGKESVTSRGYLEDEAAHAEIAFFNTILPKESSLR
YNVTWYVSSSPCVTCADRITETLKKNKNLRLTIMVGRLFMWEEPEMQAALKKMKS
AGCKLRIMKPQDFEYVWQNFVEQEAKAFVPWEDIQENFQYYEEKLAE
>A0A0Q3TEK9_AMAAE/51-219
(SEQ ID NO: 598)
FKFQFRNVEYSGRNKTFLCYVVETQGKESATSRGYLEDEAAHAEMAFFNTILPKDSS
LRYNITWYVSSSPCVTCAERIIETLKKNKNLRLTIMVGRLFMWEEPEMQAALKNMKS
AGCKLRIMKPQDFEYVWQNFVEQEAKDFVPWEDIQENFNYYEEKLAE
>FINNK5_CHICK/51-219
(SEQ ID NO: 599)
FKFQFRNVEYSGRNKTFLCYVVETQGKESKTSRGYLEDEASHAEIAFFNTILPKESSL
RYNITWYVSSSPCVTCADRISETLRKNKNLRLTIMVGRLFMWEEPEMQAALKKMKS
AGCKLRIMKPQDFEYVWQNFVEQEAKAFVPWEDIQENFQYYEEKLAE
>F6PKH9_ORNAN/19-179
(SEQ ID NO: 600)
FKFQFRNVEYSGRNKTFLCYVVETQGKENQTARGYLEDEAAHAEEAFFNSILPADQA
LKYNVTWYVSSSPCAACADRIADTLRRTPNLRLLLLVGRLFMWEEPEIQAALKKLK
AAGCKLRIMKPQDFEYVWQNFVEQEAKAFVPWEDIQENFLY
>F7EXQ1_MONDO/49-218
(SEQ ID NO: 601)
FKFQFRNVEYSGRNKTFLCYVVEVQGKQGQISRGYLEDEAAHAEEAFFKTILPTDPA
LRYNVTWYVSSSPCAACADRISSTLSKTKNLKMLLLVGRLFMWEEPEIKTALKKLKE
SGCKLRIMKPQDFEYVWQNFVEQESKAFVPWEDIQENFLYYEEKLAE
>G3WZU9_SARHA/50-219
(SEQ ID NO: 602)
FKFQFRNVEYSGRNKTFLCYVLEVQGKQGQMSRGYLEDETAHAEEAF
FKTILPTDPALRYNVTWYVSSSPCAACADRISSTLSKNKNLRMLLLVGRLFMWEEPEI
KAALKRLKEAGCKLRIMKPQDFEYVWQNFVEQESKAFVPWEDIQENFLYYEEKLAE
>I3N513_ICTTR/52-221
(SEQ ID NO: 603)
FKFQFRNVEYSGRNKTFLCYVVEAQGKQVQASRGYLEDEAAHAEEAFFNTILPTDPS
LRYNVTWYVSSSPCAACADRIIKTLGKTKNLRLLILVGRLFMWEEPEIQTALKKLKE
AGCKLRIMKPQDFEYIWQNFVEQESKAFEPWEDIQENFLYYEEKLAD
>A0A0B4J211_PONAB/52-221
(SEQ ID NO: 604)
FKFQFRNVEYSGRNKTFLCYVVEAQGKQVQASRGYLEDEAAHAEEAFFNTILPADP
ALRYNVTWYVSSSPCAACADRIIKTLSKTKNLRLLILVGRLFMWEELEIQDALKKLK
EAGCKLRIMKPQDFEYVWQNFVEQESKAFQPWEDIQENFLYYEEKLAD
>F6QZ00_CALJA/52-221
(SEQ ID NO: 605)
FKFQFRNVEYSGRNKTFLCYVVEAQGKQVQATRGYLEDEAAHAEEAFFNTILPADP
ALRYNVTWYVSSSPCAACADRITKTLSKTKNLRLLILVGRLFMWEEPEIQAALKKLK
EAGCKLRIMKPQDFEYIWQNFVEQESKAFQPWEDIQENFLYYEEKLAD
>G3TH88_LOXAF/52-221
(SEQ ID NO: 606)
FKFQFRNVEYSGRNKTFLCYVVEAQGKQVQASRGYLEDEAAHAEEAFFNTILPADP
ALRYNVTWYVSSSPCAACADRIIKTLNKTKNLRLLILVGRLFMWEEPEIQAALKKLK
EAGCKLRIMKPQDFDYVWQNFVEQESKAFEPWEDIQENFQYYEEKLAD
>H0WL56_OTOGA/52-221
(SEQ ID NO: 607)
FKFQFRNVEYSGRNKTFLCYVVEVQAKQVQASRGYLEDEAAHAEEAFFNTILPADP
ALKYNVTWYVSSSPCAACADHIIKTLNKTKNLRLLILVGRLFMWEEPEIQAALKKLK
EAGCKLRIMKPQDFEYIWQNFVEQESKAFEPWEDIQENFLYYEEKLAD
>L9KUD0_TUPCH/52-221
(SEQ ID NO: 608)
FKFQFRNVEYSGRNKTFLCYVVEAQAKQVQATRGYLEDEAAHAEEAFFNTILPADP
ALRYNVTWYVSSSPCAACADRIIKTLSKTKNLRLLILVGRLFMWEEPEIQAALRKMR
EAGCKLRIMKPQDFEYIWQNFVEQESKAFEPWEDIQENFLYYEEKLAD
>G319V7_CRIGR/52-221
(SEQ ID NO: 609)
FKFQFRNVEYSGRNKTFLCYVVEAQSKQVQATRGYLEDEGAHAEEAFFNTILPADPA
LKYNVTWYVSSSPCAACADRILKTLSKAKNLRLLILVGRLFMWEEPEVQAALKKLK
EAGCKLRIMKPQDFEYIWQNFVEQESKAFEPWEDIQENFLYYEEKLAD
>ABEC2_MOUSE/52-221
(SEQ ID NO: 610)
FKFQFRNVEYSGRNKTFLCYVVEVQSKQAQATQGYLEDEGAHAEEAFFNTILPADP
ALKYNVTWYVSSSPCAACADRILKTLSKTKNLRLLILVSRLFMWEEPEVQAALKKLK
EAGCKLRIMKPQDFEYIWQNFVEQESKAFEPWEDIQENFLYYEEKLAD
>F7AZT6_HORSE/52-221
(SEQ ID NO: 611)
FKFQFRNVEYSGRNKTFLCYVVEAQSKQVQASRGYLEDEAAHAEEAFFNTIMPADP
ALRYNVTWYVSSSPCAACADRIIKTLSKTKNLRLLILVGRLFMWEEPEIQAALKKLK
EAGCRLRIMKPQDFEYVWQNFVEQESKTFEPWEDIQENFLYYEEKLAD
>ABEC2_BOVIN/52-221
(SEQ ID NO: 612)
FKFQFRNVEYSGRNKTFLCYVVEAQSKQVQASRGYLEDETNHAEEAFFNSIMPTDPA
LRYMVTWYVSSSPCAACADRIVKTLNKTKNLRLLILVGRLFMWEEPEIQAALRKLKE
AGCRLRIMKPQDFEYIWQNFVEQESKAFEPWEDIQENFLYYEEKLAD
>S9WK76_CAMFR/64-233
(SEQ ID NO: 613)
FKFQFRNVEYSGRNKTFLCYVVEAQSKQVQATRGYLEDEAAHAEEAFFNTIMPTDP
ALRYIVTWYVSSSPCAACADRIIKTLNKTKNLRLLILVGRLFMWEEPEIQAALRKLKE
AGCKLRIMKPQDFEYVWQNFVEQESKAFEPWEDIQENFLYYEEKLAD
>F1RVM1_PIG/52-221
(SEQ ID NO: 614)
FKFQFRNVEYSGRNKTFLCYVIEAQSKQVQATRGYLEDEAAHAEEAFFNTILPADPA
VRYVVTWYVSSSPCAACADRIIKTLNKAKNLRLLILVGRLFMWEEPEIQAALRRLKE
AGCRLRIMKPQDFEYVWQNFVEQESKAFEPWEDIQENFLYYEEKLAD
>H0V2E8_CAVPO/56-225
(SEQ ID NO: 615)
FKFQFRNVEYSGRNKTFLCYVVEVQSKQVQASRGYLEDEAAHAEEAFFNSVLPADP
ALRYNVTWYVSSSPCAACADRIIKTLGKTKNLRLLILVGRLFMWEEPEIQAALRKLK
EAGCRLRIMKPQDFEYVWQNFVEQESKAFEPWEDIQENFLYYEDKLAD
>A0A091DJF3_FUKDA/59-228
(SEQ ID NO: 616)
FKFQFRNVEYSGRNKTFLCYVVEVQSKQVQASRGYLEDEAAHAEEAFFNTILPADPA
LRYSVTWYVSSSPCVACADRIVKTLGKTKNLRLLILVGRLFMWEEPEIQAALRKLKE
AGCRLRIMKPQDFEYVWQNFVEQESKAFEPWEDIQENFLYYEEKLAD
>G5BNG8_HETGA/56-225
(SEQ ID NO: 617)
FKFQFRNVEYSGRNKTFLCYVVEVQCKQVQASRGYLEDEAAHAEEAFFNTILPADP
ALRYNVTWYVSSSPCVACADRIIKTLAKTKNLRLLILVGRLFMWEEPEMQAALRKL
KEAGCRLRIMKPQDFEYVWQNFVEHESKAFEPWEDIKENFLYYEEKLAD
>G1PHZ8_MYOLU/58-227
(SEQ ID NO: 618)
FKFQFRNVEYSGRNKTFLCYVIEVQDKQVQASRGYLEDETAHAEEAFFNTVVPTDPA
LRYNVTWYVSSSPCVACADRIIKMLSKTKNLRLLILVGRLFMWEEPAMQAALKKLK
EAGCRLRIMKPQDFEYIWQNFVEQESKAFEPWEDIQENFLYYEEKLAD
>G1U7Q3_RABIT/55-224
(SEQ ID NO: 619)
FKFQFRNVEYSGRNKTFLCYVVEVQGKQVQATRGYLEDEAAHAEEAFFNTILPADP
ALRYNVTWYVSSSPCAACADRIIRTLGKTKNLRLLILVGRLFMWEEPEIQAALKKLR
EAGCRLRIMKPQDFEYVWQNFVEQEAKAFEPWEDIQENFLYYEEKLAD
>M3W1K8_FELCA/61-230
(SEQ ID NO: 620)
FKFQFRNVEYSGRNKTFLCYVVEAQGKQVQASRGYLEDEAAHAEEAFFNTILPADP
AVRYNVTWYVSSSPCAACADRIVRTLGKTKNLRLLILVGRLFMWEEPDVQAALRRL
KEAGCRLRIMKPQDFEYVWQNFVEQESKAFEPWEDIQENFLYYEEKLAD
>GILCS0_AILME/80-249
(SEQ ID NO: 621)
FKFQFRNVEYSGRNKTFLCYVVEAQGKQVQATRGYLEDEAAHAEEAFFNTILPADP
ALRYTVTWYVSSSPCAACADRIARTLGQTKNLRLLILAGRLLLWEEPDVRAALRRLA
EAGCRLRVMKPQDFEYVWQQFVEQEPKAFEPWEDIQENFLYYEEKLAD
>M3YQH9_MUSPF/61-230
(SEQ ID NO: 622)
FKFQFRNVEYSGRNKTFLCYVVETQAKQVQATRGYLEDETNHAEEAFFNTILPSDPA
LRYNVTWYVSSSPCAACADRILRTLGKTKNLRLLILVGRLFMWEEPEVQAALRKLK
EAGCRLRIMKPQDFEYVWQNFVEQESKAFEPWGDIQENFLYYEEKLAD
>E2RDL7_CANLF/52-221
(SEQ ID NO: 623)
FKFQFRNVEYSGRNKTFLCYVVEAQGKQVQASRGYLEDEAAHAEEAFFNTILPTDPA
LRYNVTWYVSSSPCAACADRIIRTLGKTKNLRLLILVGRLFMWEEPEVQAALRKLKE
AGCRLRIMKPQDFEYVWQNFVEQESKAFQPWEDIQENFLYYEEKLAD
>S7PKW6_MYOBR/115-291
(SEQ ID NO: 624)
FYFHFKNCPDHGRNGCYLCYEVKRQRGLPLVGTGVFENEPKHTEICFLNWFKTQQN
LSREEKYHVTWFMSWSPCFQCARQVVEFLKDHEYVQLSIFVARLYYSSRPEYQQGL
RSLQGAGAQVAIMTPDDFAYCRKVFVHDHKPFRYWKGIYINSCSLSKTLED
>S7NDM8_MYOBR/6-177
(SEQ ID NO: 625)
FNSNFKNLDGGCKSTFLCFEVEREDGSVLYQNGVFRNQHAELCFIEWFHEKVLCPDA
QYHVTWYISWSPCFECAEQVADFLNENENVDLSISAARLYLCEDEDEQGLQDLVAT
GAKVAMMAPEDFKYCWDNFVYNGWQFTYWKNVRRNYGRLQEKLDE
>S7PKW6_MYOBR/281-406
(SEQ ID NO: 626)
NSCSLSKTEDILRHAELCFLDWFREKVLCPDAQYHVTWYISWSPCFECAEQVADFLN
ENENVDLSISAARLYLCEDEDEQGLQDLVATGAKVAMMAPEDLTAKMVPDETPMF
DPS
>T0NHJ8_CAMFR/406-573
(SEQ ID NO: 627)
FSFHFKNLMFAGRNCTYLCYQVKREHCSPVPDKGVFQNEPCHAELCFLSWFNKRLS
PDECYHITWFMSWSPCFACTEQVAKFLEKNRNVRLSIFAARLYYFWQPAVQQGLRR
LHGVGACVGIMSYQDFKYCWENFVYNRMPFKPWEKQCENSKILVTKLEE
>F7DDE1_HORSE/12-179
(SEQ ID NO: 628)
FSFHFRNLEFAGRNCSYICYRVEGLSGSPGSEQGVFLNECRHAELCFLHWFRGRLSPD
EYYHVTWFISWSPCSNCAREVAEFLKRHRNVELSIFAARLYYWQRNKPDLRNLCSS
GAQLAIMFYQDFRYCWDNFVHNGREFIPWEKINVNSRLLATNLEE
>F7B644_HORSE/190-346
(SEQ ID NO: 629)
FSFHFRNLKFAGRKCSYLCYRVEGLSGSPGSEQGVFLNERRHAELCFLDWFRVRLSP
DEYYRVTWFISWSPCSYCAREVADFLKQYRNVKLSIFAARLYYCRDHAQGLRSLCSS
GAQLAIMFFWDFRYCWDNFVHNGRDFIPW
>F1MP61_BOVIN/29-199
(SEQ ID NO: 630)
FYFQFCNLLYARRNCSYICYKVERKYHSRAFDWGVFHNQRCHTELRFLSWFHAEKL
RPNERYHITWFMSWSPCMKCAKEVADFLGRHQNVTLSIFTARLYNFQEEGSRQGLL
RLSDQGAHVDIMSYQEFKYCWKKFVNSRRPFRPWKKLYRNYQRLVEELED
>L5K8J0_PTEAL/256-426
(SEQ ID NO: 631)
FFFEFQNLLYAGRKSSYLCFQVERQHSSPVSDWGVFENQPYHAELCFLNWFRAEKLS
PYEHYDVTWFLSWSPCSTCAEEIAIFLSNHKNVRLSIFVSRIYYFWKPAFRQGLQELD
HLGVQLDAMSFDDFKYCWENFVDNGMPFRCWKKVHRNYKFVLRKLNE
>M3W3R0_FELCA/20-189
(SEQ ID NO: 632)
FRFHFPNLLYAGRKLCYLCFQVETDYFSCDSDRGVFRNKRCHAEQCFLSWFRDQYP
CRDEYYNVTWFLSWSPCPTCAEEVVEFLEEYRNLTLSIFTSRLYYFYHPNYQQGLRK
LWDAGVQLDIMSCDDFEHCWDNFVDHGMRFQRRNLLKDYDFLAAELQE
>G1LWD3_AILME/20-186
(SEQ ID NO: 633)
FFFQFPSLCYAGRKFCYLCFQVGRGHPSDWGVFRNKPYHAESCFLSWFRAQNLSPDE
DYHVTWFSSWSPCHTCADEVVEFLGQYRHVTLSIFAARLYYFWDPPFQNGLRRLQS
AGVRLDIMSFADYKRCWENFVDHGMRFQSRNLLRHRDLLASRLEN
>L9KTG4_TUPCH/9-177
(SEQ ID NO: 634)
FYFHFQNLLFAGRNTTFLCCRVDKERHGTVLVSGVFTHQYHAESHFLLWFQKNFLSL
DKDFQVTWYLSWSPCPACAKQVADFLAVHRNVSLTIFSARLYYFWDPEFRDGLHRL
FEKGARVAIMSPKDFENCWEGFVFNGRDFRPWDNMVENYQSLRITLQE
>I3M955_ICTTR/14-186
(SEQ ID NO: 635)
FLFHFRNLRWAGRNNTFLCYQVDRERDSTVIHRGVFKTQRLHAELCFLYWLHDYPL
FPDQHFHITWFISWSPCSDCAQQVAAFLASHSNLSLTVYTARLYYFWKHSYQEGLRA
LQREGARVEIMSIREFEHCWENFVYPGRPFRPWKNLFRNYYFQVKKLQK
>G312J2_CRIGR/40-210
(SEQ ID NO: 636)
FYFHFKNLRYALRKNTFLCYEVNRECNELVLCQGVFRKENLHAEVCFLYWFHTQVL
PPDEKYKITWYVSWSPCNECAEKVASFLDTHRNLSLAIFSSRLYYFWDPDYQDKLRR
LNQAGAQIAAMDFPEFEKCWNKFVDNGKSFRPWKRLKINFRFQDNKLRD
>F7EWS7_RAT/40-210
(SEQ ID NO: 637)
FYFHFKNVRYAGRKNNFLCYEVNGDCALPVLRQGVFRKQHIHAELCFIYWFHDKVL
SPMEEFKVTWYMSWSPCSKCAEQVARFLAAHRNLSLAIFSSRLYYYLNPNYQQKLC
RLIQEGVHVAAMDLPEFKKCWNKFVDNGQPFRPWMRLRINFSFYDCKLQE
>ABEC3_RAT/26-195
(SEQ ID NO: 638)
FKFHFKNLRYADRKDTFLCYEVTRDCDSPVLHHGVFKNKNIHAEICFLYWFHDKVL
SPREEFKITWYMSWSPCFECAEQVLRFLATHHNLSLDIFSSRLYNIRDPENQQNLCRL
VQEGAQVAAMDLYEFKKCWKKFVDNGRRFRPWKKLLTNFRYQDSKLQE
>ABEC3_MOUSE/26-195
(SEQ ID NO: 639)
FKFHFKNLGYAGRKDTFLCYEVTRDCDSPVLHHGVFKNKNIHAEICFLYWFHDKVL
SPREEFKITWYMSWSPCFECAEQIVRFLATHHNLSLDIFSSRLYNVQDPETQQNLCRL
VQEGAQVAAMDLYEFKKCWKKFVDNGRRFRPWKRLLTNFRYQDSKLQE
>F6XHA6_MACMU/213-383
(SEQ ID NO: 640)
FYFHFENLQKAGRNETWLCFAVEIKQHSTVWKTGVFRNQHCHAERCFLSWFCDNTL
SPKKNYQVTWYISWSPCPECAGEVAEFLATHSNVKLTIYTARLYYFWDTDYQEGLR
SLSEEGASMEIMGYEDFKYCWENFVYNGEPFKPWKGINTNFRFLERRLWK
>A0A096NK50_PAPAN/213-383
(SEQ ID NO: 641)
FYFHFKNLRTADRNETWLCFAVEIKQRSTVWRTGVFRNQHCHAERCFLSWFRDNPL
SPKKNYQVTWYTSWSPCPECAGEVAEFLARYSNVQLTIYTARLYYFWDTDYQEGLR
SLSEEGASVEIMGYEDFKYCWENFVCDGEPFKPWKGINTNFRFLERHLRK
>G3RD21_GORGO/200-370
(SEQ ID NO: 642)
FYFHFKNLRKAGRNESWLCFTMEVKHHSPVWKRGVFRNQHCHAERCFLSWFCDDI
LSPNTNYQVTWYTSWSPCPECAGEVAEFLARHSNVNLTIFTARLYYFWDTDYQEGL
RSLNQEGASVKIMGYKDFKYCWENFVYNDEPFKPWKGLKYNFLFLDSKLQE
>A0A096NK49_PAPAN/167-338
(SEQ ID NO: 643)
FYFHFKNLRKAGRNESLLCFTMEIKQCSTVWKRGVFRNQHCHAERCFLSWFCEDILS
PNTDYQVTWYTSWSPCLDCAGEVAEFLARHSNVKLAIFAARLYYFWDTDYQQGLR
SLSEEGASVQIMGYEDFKYCWENFVYNDEPFKPWKGLKYNFLFLDSKLQE
>A0A0D9R238_CHLSB/197-367
(SEQ ID NO: 644)
FYFHFKNLRKAGRNESWLCFTMEIKQCSTVWKRGVFRNQRCHAERCFLSWFCEDIL
SPNTDYQVTWYTSWSPCLDCAGEVAEFLARHSNVKLTIYTARLYYFWHTDYQQGL
RSLSEEGASVEIMGYEDFKCCWENFVYNDEPFKPWKGLKYNFLFLDSKLQE
>G3RRB4_GORGO/39-209
(SEQ ID NO: 645)
FYFQFKNLWEADRNETWLCFTVEGKRRSVVWKTGVFRNQHCHAERCFLSWFCDDI
LSPNTNYQVTWYTSWSPCPECAGEVAEFLARHSNVNLTIFTARLYYFWDTDYQEGL
RSLNQEGASVKIMGYKDFKYCWENFVYNDEPFKPWKGLKYNFLFLDSKLQE
>G3RUE8_GORGO/17-187
(SEQ ID NO: 646)
FYFQFKNLWEADRNETWLCFTVEGKRRSVVWKTGVFRNQHCHAERCFLSWFCDDI
LSPNTNYEVTWYTSWSPCPECAGKVAEFLARHSNVNLTIFTARLCYFWDTDYQEGL
RSLNQEGASVKIMGYKDFVSCWKNFVYSDEPFKPWKGLQTNFRLLKRRLRE
>Q6ICH2_HUMAN/17-199
(SEQ ID NO: 647)
FYDNFENEPILGRSYTWLCYEVKIRGRSNLWDTGVFRGEHCHAERCFLSWFCDDILS
PNTNYEVTWYTSWSPCPECAGEVAEFLARHSNVNLTIFTARLCYFWDTDYQEGLCS
LSQEGASVKIMGYKDFVSCWKNFVYSDEPFKPWKGLQTNFRLLKRRLRE
>ABC3D_HUMAN/213-383
(SEQ ID NO: 648)
FYFHFKNLLKAGRNESWLCFTMEVKHHSAVRKRGVFRNQHCHAERCFLSWFCDDIL
SPNTNYEVTWYTSWSPCPECAGEVAEFLARHSNVNLTIFTARLCYFWDTDYQEGLCS
LSQEGASVKIMGYKDFVSCWKNFVYSDEPFKPWKGLQTNFRLLKRRLRE
>ABC3C_GORGO/17-187
(SEQ ID NO: 649)
FYFQFKNLWEADRNETWLCFTVEGKRRSVVWKTGVFRNQHCHAERCFLSWFCDDI
LSPNTNYQVTWYTSWSPCPECAGEVAEFLARHSNVNLTIFTARLYYFQDTDYQEGLR
SLSQEGVAVKIMDYKDFKYCWENFVYNDEPFKPWKGLKYNFRFLKRRLQE
>ABC3C_HUMAN/17-187
(SEQ ID NO: 650)
FYFQFKNLWEADRNETWLCFTVEGKRRSVVWKTGVFRNQHCHAERCFLSWFCDDI
LSPNTKYQVTWYTSWSPCPDCAGEVAEFLARHSNVNLTIFTARLYYFQYPCYQEGL
RSLSQEGVAVEIMDYEDFKYCWENFVYNNEPFKPWKGLKTNFRLLKRRLRE
>H2P4E8_PONAB/17-187
(SEQ ID NO: 651)
FYFQFKNLWEADRNETWLCFTVEVKHHTVVWKRGVFRNQHCHAESCFLSWFCNNI
LSPNTNYRVTWYASWSPCPECAGEVAKFLARHSNVKLTIFTARLYYFQNPYYQQGL
RRLSQEGVAVGIMDYEDFKDCWENFVYSDEPFKPWKGINTNFRLLKKRLRE
>A0A096NK46_PAPAN/17-187
(SEQ ID NO: 652)
FYFQFKNLWEANRNETWLCFTVEVKQRSTVWERGVFQNQHCHAERCFLSWFCEDIL
SPNTDYQVTWYTSWSPCPECAGEVAEFLARHNNVMLTIYTARLYYSQDPNYQQGLR
SLSEKGVSVKIMDYEDFKYCWENFVYNDEPFKPWKGLKYNFLFLDSKLQE
>A0A0D9R255_CHLSB/17-187
(SEQ ID NO: 653)
FYFQFKNLREANRNETWLCFTVEGRHHLTVWKTGVFRNQHCHAEKCFLSWFCKNIL
SPNTDYQVTWYTSWSPCPECAREVAKFLARHNNVMLTIYTARLYYSQCPNYQQGLR
SLSEKGVSVEIMDYEDFKYCWQNFVYDGEPFKPWKGLKTSFRFLKRCLRE
>F6XHB3_MACMU/37-207
(SEQ ID NO: 654)
FYFQFKNLREANRNETWLCFAVEIKQRSTVLKTGVFRNQHCHAERCFLSWFCEDILS
PNTDYQVTWYTSWSPCLDCAGEVAKFLARHNNVMLTIYTARLYYSQYPNYQQGLR
SLSEKGVSVKIMDYEDFKYCWEKFVYDGEPFKPWKGLKTSFRFLKRRLRE
>H0VE76_CAVPO/1-121
(SEQ ID NO: 655)
LHAELSFLSWFHDTELSFDENYKVTWYMSWSPCPECAKEIVTFLDNHHNVTLTIYVA
RLYYHWNPTYKEGLRALVQGGTRLYTMAFPEFEDCWSLFVNETFRPWENFHKCCSL
QDKTLQK
>G5AYU4_HETGA/17-183
(SEQ ID NO: 656)
FYFHFENLPYAGRNKTFLCYEVKRRDNKLHKGVVQNQLSRTELSFISCFHATELCLD
ETYKVTWYISWSPCVECAEEIVKFLANHRNVFLTVFIARLYYYREHTFKEGLQALDN
GGVQMHMMCLQDFKDCWSLFVSETFRPWKGLRKYYLFQNKTLKQ
>H0VZF7_CAVPO/17-183
(SEQ ID NO: 657)
FYFHFENLPNAGRHKTFLCFEVKNNELHKGFFLNQLHAELRFLSWLHDTCLCPYEYY
QVTWYMSWSPCVECAEELTTFLAGHRNVTLTIYVARLYYHQFPVYKNRLQALIKKG
ATVKVMFFRDFLYCWRRFVYNFKRFYDWPNLHKNSLHYYKTLQH
>G5AYU3_HETGA/12-181
(SEQ ID NO: 658)
FYFHFENLPDPGWNKTFLCYEVKRQDQKLRKGVFQNQPLHAELRFLSWFHDTLLCP
LGSYQVTLYVSWSPCSECAEELTTFLAGHRNVTMTIYVAQLYYCNKSPNREGLKILI
AEDARLRVMFYDEFLYCWRNFVKNYNNFDPWSLLDENSRYHNRILQN
>G5AYU5_HETGA/20-189
(SEQ ID NO: 659)
FRFYFRNLRCAGRNKTFLCYEVKRRDNKLHKGVVLNQPLHAELRFLSWFHDTLLCP
LGSYQVTLYVSWSPCSECAEELTTFLAGHRNVTMTIYVAQLYYCNKSPNREGLKILI
AEDARLRVMFYDEFLYCWRNFVKNYNNFDPWSLLDENSRYHNRILQN
>A0A091EM42_FUKDA/42-211
(SEQ ID NO: 660)
FKFHFENLPNPGWHKTFLCYTIESQKNRLRNGVFQNQRLHAELRFLSWFHDNWLCP
GNSYRVTFYMSWSPCSECAEELTTFLAGHRNVTLTIFFSKLYYCDDSSNREGLKTLA
AGGARLFVMFDTDFSYCWTNFVNCYNYFEPWPLLDDNSKYCNRILQK
>A0A091EM42_FUKDA/225-394
(SEQ ID NO: 661)
FYFHFNNLCFAGRNKTFLCYTIESKKNRLRNGVFQNQRLHAELRFLSWFHDNWLCP
GNSYQVTLYMSWSPCSECAEELTTFLAGHRNVTLTIFFSKLYCCDDSPHREGLKTLA
AGGARLFVMFDTDFSYCWTNFVNCYNYFEPWPLLDDNSKYYKSILQK
>H0XYD2_OTOGA/17-183
(SEQ ID NO: 662)
FNCHFNNRPYLRRNDTWLCFEVKTSSNSPGFYSGVFQNQPWHTELCILTWARPMLS
HHHFYQITWYMSWSPCANCAWQVAAFLATHENVSLTIYTAHIYYMWRQDYRQGM
LRMIEEGTRVYIMFSKEFQHCWENFVDHGMCWNRVKKNYEFLVTQLNE
>H0XYD2_OTOGA/192-348
(SEQ ID NO: 663)
FYNQFNNTPVPGRTDTWLCFEVKNNSNSPGFHRGSSENRHAEVGFLTWFQKEMPPN
HHYEVTWYISWSPCVHCAWHVVNFLTSNPNMTLTIFAARLYYIYHPEEGTKVHIVSL
KEFKYCWAKLVYNGMRFMPWYQFNENYQLLVTQLKK
>A0A096N7U5_PAPAN/17-167
(SEQ ID NO: 664)
FYYNFENRSVLGWNTTWLCHKVKTKDPSKLLDTRIFGGQHHPEMRFLDWFCKYISR
SPCPEYAEKVAEFLVKNGKVTLTIFVAHLYYFWEPDYQNGVRPHASMTIMNYDEFQ
HCWDKFVYNRMLFDPWKQLNTNYALLHSMLGE
>H9KW44_CALJA/2-132
(SEQ ID NO: 665)
QHHPEMRFLHWFRKWKLHSDQEYEVTWFVSWSPCPVCARNVAEFLTEDGKVTLTIF
VARLYYFWIPHYREELRRLCQPRATMKIMSYGEFQHCWDKFVDNRLYKPWNKLPK
HYTLLHITLGE
>ABC3G_PAPAN/17-191
(SEQ ID NO: 666)
FFYNFNNRPILRRNTVWLCYEVKTRGPSMPWDAKIFRGQKYHPEMRFLHWFRKWR
QLHRDQEYEVTWYVSWSPCTGCANSVATFLAEDPKVTLTIFVARLYYFWKPDYQEA
LRVLCQPHATMKIMNYNEFQHCWNKFVRGREPFEPWENLPKHYTLLHATLGE
>G7PFK4_MACFA/13-187
(SEQ ID NO: 667)
FFYNFNNRPILRRNTVWLCYEVKTRGPSVPWGTKIFRGQKYHPEMRFLRWFHKWRQ
LHHDQEYKVTWYVSWSPCTRCANSVATFLAKDPKVTLTIFVARLYYFWKPDYQQA
LRILCQPHATMKIMNYNEFQDCWNKFVDGGKPFKPRNNLPKHYTLLQATLGE
>A0A0D9R229_CHLSB/28-202
(SEQ ID NO: 668)
FVYYFNNRPILGRNIVWLCCEVKTKDPSGPLDANIFQGEKDHPEMKFLHWFRKWRQ
LHRDQEYEVTWYVSWSPCTRCANSVATFLAEDPKVTLTIFVARLYYFWKPHYQEAL
RILCQPHATMKIMNYNEFQHCWNEFVDGGKPFKPRKNLPKHYTLLHATLGE
>ABC3G_MACMU/10-184
(SEQ ID NO: 669)
FVSNFNNRPILGLNTVWLCCEVKTKDPSGPLDAKIFQGKKYHPEMRFLRWFHKWRQ
LHHDQEYKVTWYVSWSPCTRCANSVATFLAKDPKVTLTIFVARLYYFWKPDYQQA
LRILCQPHATMKIMNYNEFQDCWNKFVDGGKPFKPRNNLPKHYTLLQATLGE
>H2P4E9_PONAB/31-202
(SEQ ID NO: 670)
FSYNFKNRPILRRNTVWLCYEVKTKGPSRPLDAKIFRGQKNHPEMRFFHWFSKWRT
LHRDQECEVTWYMSWSPCTKCTRNVATFLAEDPKVTLTIFVARLYYFWDPDYQEAL
RSLCRPRANMKIMNYDEFQHCWNKFVYSRELFEPWNNLPKYYIPLHKV
>ABC3G_HUMAN/17-190
(SEQ ID NO: 671)
FSYNFYNRPILRRNTVWLCYEVKTKGPSRPLDAKIFRGQKYHPEMRFFHWFSKWRK
LHRDQEYEVTWYISWSPCTKCTRDMATFLAEDPKVTLTIFVARLYYFWDPDYQEAL
RSLCQPRATMKIMNYDEFQHCWSKFVYSRELFEPWNNLPKYYILLHIMLG
>ABC3G_GORGO/17-190
(SEQ ID NO: 672)
FSYNFNNRPILRRNTVWLCYEVKTKDPSRPLDAKIFRGQKYHPEMRFFHWFSKWRK
LHRDQEYEVTWYISWSPCTKCTRNVATFLAEDPKVTLTIFVARLYYFWDQDYQEAL
RSLCQPRATMKIMNYDEFQHCWSKFVYSRELFEPWNNLPKYYMLLHIMLG
>G3RD21_GORGO/17-186
(SEQ ID NO: 673)
FSYNFNNRPILRRNTVWLCYEVKTKGPSRPLDAKIFRGQQYHAEMCFLSWFCGNQLP
AYKCFQITCFVSWTPCPDCVAKLAEFLAEHPNVTLTISAARLYYYWERDYRRALRRL
RQAGARVKIMDDEEFAYCWENFVYSGQPFMPWHKFDDNYAFLHRTLKE
>ABC3F_HUMAN/17-186
(SEQ ID NO: 674)
FSYNFYNRPILRRNTVWLCYEVKTKGPSRPLDAKIFRGQEHHAEMCFLSWFCGNQLP
AYKCFQITWFVSWTPCPDCVAKLAEFLAEHPNVTLTISAARLYYYWERDYRRALCR
LSQAGARVKIMDDEEFAYCWENFVYSGQPFMPWYKFDDNYAFLHRTLKE
>A0A096NK49_PAPAN/1-153
(SEQ ID NO: 675)
WFCFEVKTRGPSMPWDAKIFRGQQYHAEMCFLSRFCGNQLPAYKRFQITWFVSWTP
CPDCVVKVAEFLAEHPNVTLTISAARLYYYWETDYRRALCRLRQAGAHVKIMDNEE
FAYCWENFVYNGQSFMPWDKFDDNYAFLHCKLKE
>A0A096NK45_PAPAN/17-187
(SEQ ID NO: 676)
FYYHFENKPILGRSYTWLCYEVKIKDPSKLWYTGVFRGQEHHAEMCFLSRFCGNQL
PAYKRFQITWFVSWNPCPDCVAKVIEFLAEHPNVTLTISTARLYYYWGRDWQRALC
RLRQAGARVKIMDYEEFAYCWENFVYNGQSFMPWDKFDDNYAFLHCKLKE
>F7FXJ2_MACMU/2-130
(SEQ ID NO: 677)
EHHAEMCFLSRFCGNQLPAYKRFQITWFVSWNPCPDCVAKVIEFLAEHPNVTLTIST
ARLYYYWGRDWQRALCRLRQAGARVKIMDYEEFAYCWENFVYNDQSFMPWYKF
NDNYAFLHRMLKE
>A0A0D9R238_CHLSB/17-183
(SEQ ID NO: 678)
FYYNFENEPILGRRYTWLCYEVKIKDPSKLWDTGVFPGQQYHAEMYFLSWFCGNQL
PAYKHFQITWFVSWNPCPDCVAKVTEFLAEHRNVTLTISAARLYYYWGKDWRRAL
CRLHQAGARVKIMDDEEFAYCWENCVYNGQPFMPWDKFDDNYAFLHLKLKE
>A0A096NK50_PAPAN/17-199
(SEQ ID NO: 679)
FDYNFENEPILGRSYTWLCYEVKIEDPSKLWDTGVFQGQQYHAEMCFLSRFCGNQLP
AYKRFQITWFVSWNPCPDCVAKVTEFLAEHPNVTLTISAARLYYYWGRDWRRALRR
LHQAGARVKIMDYEEFAYCWENFVYNGQSFMPWDKFDDNYAFLHCKLKD
>F6XHA6_MACMU/17-199
(SEQ ID NO: 680)
FNYNFENEPILGRSYTWLCYEVKIKDPSKLWDTGVFRGQQYHAEMCFLSWFCGNQL
PAYKRFQITWFVSWNPCPDCVAKVTEFLAEHPNVTLTISAARLYYYWGKDWRRALR
RLHQAGALVKIMDYEEFAYCWENFVYNGQSFMPWDKFDDNYASLHCKLKE
>A0A0D9R289_CHLSB/17-187
(SEQ ID NO: 681)
FYYHFENEPILGRSYTWLCYEVKIKDPSKLWDTGVFRGQEHHAEMCFLSWFCGNQL
PAHKRFQITWFVSWTPCPDCVAKVAEFLAEYPNVTLTISAARLYYYWETDYRRALC
RLRQAGARVKIMDYEEFAYCWENCVYNGQPFMPWYKFDDNYAFLHHKLKE
>GIRYY7_NOMLE/17-187
(SEQ ID NO: 682)
FYYNFENEPILRRSYTWLCYEVKIKDPSKLWDTGVFRGQEYHAEMCFLSWFCGNQL
PAYKRFQITWFVSWTPCPDCVAKVAVFLAEHPNVTLTISAARLYYYWEKDWQRALC
RLSQAGARVKIMDYEEFEYCWENFVYNGEPFMPWYKFDDNYAFLHHTLKE
>H2P4E7_PONAB/17-187
(SEQ ID NO: 683)
FYYDFENEPILRRNYTWLCYEVKIKDPSKLWDTGVFRGQEHHAEMCFLSWFCGNQL
SAYERFQITWFVSWTPCPDCVAKLAEFLAEHPNVTLTVSAARLYYYWERGYRRALR
RLRQAGAHVKIMDYEEFAYCWENFVYNGQPFMPWYKFDDNYAFLHHTLKE
>H2QLP5_PANTR/17-187
(SEQ ID NO: 684)
FYYNFENEPILGRSYTWLCYEVKIRGHSNLWDTGVFRGQEHHAEMCFLSWFCGNQL
SAYKCFQITWFVSWTPCPDCVAKLAKFLAEHPNVTLTISAARLYYYWERDYRRALC
RLSQAGARVKIMDDEEFAYCWENFVYNGQPFMPWYKFDDNYAFLHRTLKE
>ABC3D_HUMAN/17-199
(SEQ ID NO: 685)
FYDNFENEPILGRSYTWLCYEVKIRGRSNLWDTGVFRGPENHAEMCFLSWFCGNRL
PANRRFQITWFVSWNPCLPCVVKVTKFLAEHPNVTLTISAARLYYYRDRDWRWVLL
RLHKAGARVKIMDYEDFAYCWENFVCNGQPFMPWYKFDDNYASLHRTLKE
>ABC3B_HUMAN/17-187
(SEQ ID NO: 686)
FYDNFENEPILGRSYTWLCYEVKIRGRSNLWDTGVFRGQQYHAEMCFLSWFCGNQL
PAYKCFQITWFVSWTPCPDCVAKLAEFLSEHPNVTLTISAARLYYYWERDYRRALCR
LSQAGARVTIMDYEEFAYCWENFVYNGQQFMPWYKFDENYAFLHRTLKE
>G3SFT2_GORGO/17-187
(SEQ ID NO: 687)
FYDNFENEPILGRSYNWLCYEVKIRGRSNLWNTGVFRGQEHHAEMCFLSWFCGNQL
PAYKCFQITWFVSWTPCPDCVAKLAEFLAEYPNVTLTISTARLYYYWERDYRRALCR
LSQAGARMKIMDYEECAYCWENFVYKGQQFMPWYKFDENYAFLHHTLKE

### Sequences 6. Sequences of Claims:

NLS sequences:
(SEQ ID NO: 1)
KRTADGSEFESPKKKRKV
(SEQ ID NO: 2)
KRTADGSEFEPKKKRKV
Cas9 for BE4max, AncBE4max and ABEmax:
(SEQ ID NO: 3)
DKKYSIGLAIGTNSVGWAVITDEYKVPSKKFKVLGNTDRHSIKKNLIG
ALLFDSGETAEATRLKRTARRRYTRRKNRICYLQEIFSNEMAKVDDSFFHRLEESFLV
EEDKKHERHPIFGNIVDEVAYHEKYPTIYHLRKKLVDSTDKADLRLIYLALAHMIKFR
GHFLIEGDLNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDDDLDNLLA
QIGDQYADLFLAAKNLSDAILLSDILRVNTEITKAPLSASMIKRYDEHHQDLTLLKAL
VRQQLPEKYKEIFFDQSKNGYAGYIDGGASQEEFYKFIKPILEKMDGTEELLVKLNRE
DLLRKQRTFDNGSIPHQIHLGELHAILRRQEDFYPFLKDNREKIEKILTFRIPYYVGPLA
RGNSRFAWMTRKSEETITPWNFEEVVDKGASAQSFIERMTNFDKNLPNEKVLPKHSL
LYEYFTVYNELTKVKYVTEGMRKPAFLSGEQKKAIVDLLFKTNRKVTVKQLKEDYF
KKIECFDSVEISGVEDRFNASLGTYHDLLKIIKDKDFLDNEENEDILEDIVLTLTLFEDR
EMIEERLKTYAHLFDDKVMKQLKRRRYTGWGRLSRKLINGIRDKQSGKTILDFLKSD
GFANRNFMQLIHDDSLTFKEDIQKAQVSGQGDSLHEHIANLAGSPAIKKGILQTVKV
VDELVKVMGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELD
KAGFIKRQLVETRQITKHVAQILDSRMNTKYDENDKLIREVKVITLKSKLVSDFRKDF
QFYKVREINNYHHAHDAYLNAVVGTALIKKYPKLESEFVYGDYKVYDVRKMIAKSE
QEIGKATAKYFFYSNIMNFFKTEITLANGEIRKRPLIETNGETGEIVWDKGRDFATVR
KVLSMPQVNIVKKTEVQTGGFSKESILPKRNSDKLIARKKDWDPKKYGGFDSPTVAY
SVLVVAKVEKGKSKKLKSVKELLGITIMERSSFEKNPIDFLEAKGYKEVKKDLIIKLP
KYSLFELENGRKRMLASAGELQKGNELALPSKYVNFLYLASHYEKLKGSPEDNEQK
QLFVEQHKHYLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQLGGD
rAPOBEC:
(SEQ ID NO: 4)
SSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAITEFLSRYP
HVTLFIYIARLYHHADPRNRQGLRDLISSGVTIQIMTEQESGYCWRNFVNYSPSNEAH
WPRYPHLWVRLYVLELYCIILGLPPCLNILRRKQPQLTFFTIALQSCHYQRLPPHILWA
TGLK
Anc689 APOBEC:
(SEQ ID NO: 5)
SSETGPVAVDPTLRRRIEPHEFEVFFDPRELRKETCLLYEIKWGTSHKI
WRHSSKNTTKHVEVNFIEKFTSERHFCPSTSCSITWFLSWSPCGECSKAITEFLSQHPN
VTLVIYVARLYHHMDQQNRQGLRDLVNSGVTIQIMTAPEYDYCWRNFVNYPPGKE
AHWPRYPPLWMKLYALELHAGILGLPPCLNILRRKQPQLTFFTIALQSCHYQRLPPHI
LWATGLK
Anc687 APOBEC:
(SEQ ID NO: 6)
SSETGPVAVDPTLRRRIEPHEFEVFFDPRELRKEACLLYEIKWGTSHKI
WRNSGKNTTKHVEVNFIEKFTSERHFCPSISCSITWFLSWSPCWECSKAIREFLSQHPN
VTLVIYVARLFQHMDQQNRQGLRDLVNSGVTIQIMTASEYDHCWRNFVNYPPGKEA
HWPRYPPLWMKLYALELHAGILGLPPCLNILRRKQPQLTFFTIALQSCHYQRLPPHIL
WATGLK
Anc686 APOBEC:
(SEQ ID NO: 7)
SSETGPVAVDPTLRRRIEPEFFNRNYDPRELRKETYLLYEIKWGKESKI
WRHTSNNRTQHAEVNFLENFFNELYFNPSTHCSITWFLSWSPCGECSKAIVEFLKEHP
NVNLEIYVARLYLCEDERNRQGLRDLVNSGVTIRIMNLPDYNYCWRTFVSHQGGDE
DYWPRHFAPWVRLYVLELYCIILGLPPCLNILRRKQPQLTFFTIALQSCHYQRLPPHIL
WATGLK
Anc655 APOBEC:
(SEQ ID NO: 8)
SSETGPVAVDPTLRRRIEPFYFQFNNDPRACRRKTYLCYELKQDGSTW
VWKRTLHNKGRHAEICFLEKISSLEKLDPAQHYRITWYMSWSPCSNCAQKIVDFLKE
HPHVNLRIYVARLYYHEEERYQEGLRNLRRSGVSIRVMDLPDFEHCWETFVDNGGG
PFQPWPGLEELNSKQLSRRLQAGILGLPPCLNILRRKQPQLTFFTIALQSCHYQRLPPH
ILWATGLK
Anc733 APOBEC:
(SEQ ID NO: 9)
SSETGPVAVDPTLRRRIEPFHFQFNNDPRAYRRKTYLCYELKQDGSTW
VLDRTLRNKGRHAEICFLDKINSWERLDPAQHYRVTWYMSWSPCSNCAQQVVDFL
KEHPHVNLRIFAARLYYHEQRRYQEGLRSLRGSGVPVAVMTLPDFEHCWETFVDHG
GRPFQPWDGLEELNSRSLSRRLQAGILGLPPCLNILRRKQPQLTFFTIALQSCHYQRLP
PHILWATGLK
UGI domain:
(SEQ ID NO: 10)
MTNLSDIIEKETGKQLVIQESILMLPEEVEEVIGNKPESDILVHTAYDES
TDENVMLLTSDAPEYKPWALVIQDSNGENKIKML
32 a.a. linker:
(SEQ ID NO: 11)
SGGSSGGSSGSETPGTSESATPESSGGSSGGS
BE4max:
(SEQ ID NO: 12)
MKRTADGSEFESPKKKRKVSSETGPVAVDPTLRRRIEPHEFEVFFDPRE
LRKETCLLYEINWGGRHSIWRHTSQNTNKHVEVNFIEKFTTERYFCPNTRCSITWFLS
WSPCGECSRAITEFLSRYPHVTLFIYIARLYHHADPRNRQGLRDLISSGVTIQIMTEQE
SGYCWRNFVNYSPSNEAHWPRYPHLWVRLYVLELYCIILGLPPCLNILRRKQPQLTF
FTIALQSCHYQRLPPHILWATGLKSGGSSGGSSGSETPGTSESATPESSGGSSGGSDKK
YSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPD
NSDVDKLFIQLVQTYNQLFEENPINASGVDAKAILSARLSKSRRLENLIAQLPGEKKN
GLFGNLIALSLGLTPNFKSNFDLAEDAKLQLSKDTYDDDLDNLLAQIGDQYADLFLA
AKNLSDAILLSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGNSRFAWMTR
KSEETITPWNFEEVVDKGASAQSFIERMTNFDKNLPNEKVLPKHSLLYEYFTVYNELT
KVKYVTEGMRKPAFLSGEQKKAIVDLLFKTNRKVTVKQLKEDYFKKIECFDSVEISG
VEDRFNASLGTYHDLLKIIKDKDFLDNEENEDILEDIVLTLTLFEDREMIEERLKTYAH
LFDDKVMKQLKRRRYTGWGRLSRKLINGIRDKQSGKTILDFLKSDGFANRNFMQLIH
DDSLTFKEDIQKAQVSGQGDSLHEHIANLAGSPAIKKGILQTVKVVDELVKVMGRHK
PENIVIEMARENQTTQKGQKNSRERMKRIEEGIKELGSQILKEHPVENTQLQNEKLYL
YYLQNGRDMYVDQELDINRLSDYDVDHIVPQSFLKDDSIDNKVLTRSDKNRGKSDN
VPSEEVVKKMKNYWRQLLNAKLITQRKFDNLTKAERGGLSELDKAGFIKRQLVETR
QITKHVAQILDSRMNTKYDENDKLIREVKVITLKSKLVSDFRKDFQFYKVREINNYH
HAHDAYLNAVVGTALIKKYPKLESEFVYGDYKVYDVRKMIAKSEQEIGKATAKYFF
YSNIMNFFKTEITLANGEIRKRPLIETNGETGEIVWDKGRDFATVRKVLSMPQVNIVK
KTEVQTGGFSKESILPKRNSDKLIARKKDWDPKKYGGFDSPTVAYSVLVVAKVEKG
KSKKLKSVKELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLFTLTNLGAPAAFKYF
DTTIDRKRYTSTKEVLDATLIHQSITGLYETRIDLSQLGGDSGGSGGSGGSTNLSDIIEK
ETGKQLVIQESILMLPEEVEEVIGNKPESDILVHTAYDESTDENVMLLTSDAPEYKPW
ALVIQDSNGENKIKMLSGGSGGSGGSTNLSDIIEKETGKQLVIQESILMLPEEVEEVIG
NKPESDILVHTAYDESTDENVMLLTSDAPEYKPWALVIQDSNGENKIKMLSGGSKRT
ADGSEFEPKKKRKV
AncBE4max 689:
(SEQ ID NO: 13)
MKRTADGSEFESPKKKRKVSSETGPVAVDPTLRRRIEPHEFEVFFDPRE
LRKETCLLYEIKWGTSHKIWRHSSKNTTKHVEVNFIEKFTSERHFCPSTSCSITWFLS
WSPCGECSKAITEFLSQHPNVTLVIYVARLYHHMDQQNRQGLRDLVNSGVTIQIMTA
PEYDYCWRNFVNYPPGKEAHWPRYPPLWMKLYALELHAGILGLPPCLNILRRKQPQ
LTFFTIALQSCHYQRLPPHILWATGLKSGGSSGGSSGSETPGTSESATPESSGGSSGGS
DKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSGGSGGSTNLSDIIEKETGKQLVIQESILMLPE
EVEEVIGNKPESDILVHTAYDESTDENVMLLTSDAPEYKPWALVIQDSNGENKIKML
SGGSKRTADGSEFEPKKKRKV
AncBE4max687:
(SEQ ID NO: 14)
MKRTADGSEFESPKKKRKVSSETGPVAVDPTLRRRIEPHEFEVFFDPRE
LRKEACLLYEIKWGTSHKIWRNSGKNTTKHVEVNFIEKFTSERHFCPSISCSITWFLS
WSPCWECSKAIREFLSQHPNVTLVIYVARLFQHMDQQNRQGLRDLVNSGVTIQIMTA
SEYDHCWRNFVNYPPGKEAHWPRYPPLWMKLYALELHAGILGLPPCLNILRRKQPQ
LTFFTIALQSCHYQRLPPHILWATGLKSGGSSGGSSGSETPGTSESATPESSGGSSGGS
DKKYSIGLAIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGE
KKNGLFGNLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALVRQQLPEKY
KEIFFDQSKNGYAGYIDGGASQEEFYKFIKPILEKMDGTEELLVKLNREDLLRKQRTF
DNGSIPHQIHLGELHAILRRQEDFYPFLKDNREKIEKILTFRIPYYVGPLARGNSRFAW
MTRKSEETITPWNFEEVVDKGASAQSFIERMTNFDKNLPNEKVLPKHSLLYEYFTVY
NELTKVKYVTEGMRKPAFLSGEQKKAIVDLLFKTNRKVTVKQLKEDYFKKIECFDS
VEISGVEDRFNASLGTYHDLLKIIKDKDFLDNEENEDILEDIVLTLTLFEDREMIEERL
KTYAHLFDDKVMKQLKRRRYTGWGRLSRKLINGIRDKQSGKTILDFLKSDGFANRN
FMQLIHDDSLTFKEDIQKAQVSGQGDSLHEHIANLAGSPAIKKGILQTVKVVDELVK
VMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDK
NRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKV
REINNYHHAHDAYLNAVVGTALIKKYPKLESEFVYGDYKVYDVRKMIAKSEQEIGK
ATAKYFFYSNIMNFFKTEITLANGEIRKRPLIETNGETGEIVWDKGRDFATVRKVLSM
PQVNIVKKTEVQTGGFSKESILPKRNSDKLIARKKDWDPKKYGGFDSPTVAYSVLVV
AKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQ
HKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGDSGGSGGSGGST
NLSDIIEKETGKQLVIQESILMLPEEVEEVIGNKPESDILVHTAYDESTDENVMLLTSD
APEYKPWALVIQDSNGENKIKMLSGGSGGSGGSTNLSDIIEKETGKQLVIQESILMLPE
EVEEVIGNKPESDILVHTAYDESTDENVMLLTSDAPEYKPWALVIQDSNGENKIKML
SGGSKRTADGSEFEPKKKRKV
TadA WT:
(SEQ ID NO: 15)
MSEVEFSHEYWMRHALTLAKRAWDEREVPVGAVLVHNNRVIGEGW
NRPIGRHDPTAHAEIMALRQGGLVMQNYRLIDATLYVTLEPCVMCAGAMIHSRIGR
VVFGARDAKTGAAGSLMDVLHHPGMNHRVEITEGILADECAALLSDFFRMRRQEIK
AQKKAQSSTD
Staphylococcus aureus TadA:
(SEQ ID NO: 16)
MGSHMTNDIYFMTLAIEEAKKAAQLGEVPIGAIITKDDEVIARAHNLR
ETLQQPTAHAEHIAIERAAKVLGSWRLEGCTLYVTLEPCVMCAGTIVMSRIPRVVYG
ADDPKGGCSGSLMNLLQQSNFNHRAIVDKGVLKEACSTLLTTFFKNLRANKKSTN
Bacillus subtilis TadA:
(SEQ ID NO: 17)
MTQDELYMKEAIKEAKKAEEKGEVPIGAVLVINGEIIARAHNLRETEQ
RSIAHAEMLVID
EACKALGTWRLEGATLYVTLEPCPMCAGAVVLSRVEKVVFGAFDPK
GGCSGTLMNLLQEERFNHQAEVVSGVLEEECGGMLSAFFRELRKKKKAARKNLSE
Salmonella typhimurium (S. typhimurium) TadA:
(SEQ ID NO: 18)
MPPAFITGVTSLSDVELDHEYWMRHALTLAKRAWDEREVPVGAVLV
HNHRVIGEGWNRPIGRHDPTAHAEIMALRQGGLVLQNYRLLDTTLYVTLEPCVMCA
GAMVHSRIGRVVFGARDAKTGAAGSLIDVLHHPGMNHRVEIIEGVLRDECATLLSDF
FRMRRQEIKALKKADRAEGAGPAV
Shewanella putrefaciens (S. putrefaciens) TadA:
(SEQ ID NO: 19)
MDEYWMQVAMQMAEKAEAAGEVPVGAVLVKDGQQIATGYNLSISQ
HDPTAHAEILCLRSAGKKLENYRLLDATLYITLEPCAMCAGAMVHSRIARVVYGAR
DEKTGAAGTVVNLLQHPAFNHQVEVTSGVLAEACSAQLSRFFKRRRDEKKALKLAQ
RAQQGIE
Haemophilus influenzae F3031 (H. influenzae) TadA:
(SEQ ID NO: 20)
MDAAKVRSEFDEKMMRYALELADKAEALGEIPVGAVLVDDARNIIGE
GWNLSIVQSDPTAHAEIIALRNGAKNIQNYRLLNSTLYVTLEPCTMCAGAILHSRIKR
LVFGASDYKTGAIGSRFHFFDDYKMNHTLEITSGVLAEECSQKLSTFFQKRREEKKIE
KALLKSLSDK
Caulobacter crescentus (C. crescentus) TadA:
(SEQ ID NO: 21)
MRTDESEDQDHRMMRLALDAARAAAEAGETPVGAVILDPSTGEVIAT
AGNGPIAAHDPTAHAEIAAMRAAAAKLGNYRLTDLTLVVTLEPCAMCAGAISHARI
GRVVFGADDPKGGAVVHGPKFFAQPTCHWRPEVTGGVLADESADLLRGFFRARRK
AKI
Geobacter sulfurreducens (G. sulfurreducens) TadA:
(SEQ ID NO: 22)
MSSLKKTPIRDDAYWMGKAIREAAKAAARDEVPIGAVIVRDGAVIGR
GHNLREGSNDPSAHAEMIAIRQAARRSANWRLTGATLYVTLEPCLMCMGAIILARLE
RVVFGCYDPKGGAAGSLYDLSADPRLNHQVRLSPGVCQEECGTMLSDFFRDLRRRK
KAKATPALFIDERKVPPEP
TadA 7.10:
(SEQ ID NO: 23)
SEVEFSHEYWMRHALTLAKRARDEREVPVGAVLVLNNRVIGEGWNR
AIGLHDPTAHAEIMALRQGGLVMQNYRLIDATLYVTFEPCVMCAGAMIHSRIGRVVF
GVRNAKTGAAGSLMDVLHYPGMNHR VEITEGILADECAALLCYFFRMPRQVFNAQ
KKAQSSTD
ABEmax:
(SEQ ID NO: 24)
MKRTADGSEFESPKKKRKVMSEVEFSHEYWMRHALTLAKRAWDERE
VPVGAVLVHNNRVIGEGWNRPIGRHDPTAHAEIMALRQGGLVMQNYRLIDATLYVT
LEPCVMCAGAMIHSRIGRVVFGARDAKTGAAGSLMDVLHHPGMNHRVEITEGILAD
ECAALLSDFFRMRRQEIKAQKKAQSSTDSGGSSGGSSGSETPGTSESATPESSGGSSG
GSSEVEFSHEYWMRHALTLAKRARDEREVPVGAVLVLNNRVIGEGWNRAIGLHDPT
AHAEIMALRQGGLVMQNYRLIDATLYVTFEPCVMCAGAMIHSRIGRVVFGVRNAKT
GAAGSLMDVLHYPGMNHRVEITEGILADECAALLCYFFRMPRQVFNAQKKAQSSTD
SGGSSGGSSGSETPGTSESATPESSGGSSGGSDKKYSIGLAIGTNSVGWAVITDEYKVP
SKKFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKKLV
DSTDKADLRLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPNFKSNFDLA
EDAKLQLSKDTYDDDLDNLLAQIGDQYADLFLAAKNLSDAILLSDILRVNTEITKAPL
SASMIKRYDEHHQDLTLLKALVRQQLPEKYKEIFFDQSKNGYAGYIDGGASQEEFYK
FIKPILEKMDGTEELLVKLNREDLLRKQRTFDNGSIPHQIHLGELHAILRRQEDFYPFL
KDNREKIEKILTFRIPYYVGPLARGNSRFAWMTRKSEETITPWNFEEVVDKGASAQSF
IERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVSGQGDSLH
EHIANLAGSPAIKKGILQTVKVVDELVKVMGRHKPENIVIEMARENQTTQKGQKNSR
ERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMNTKYDEND
KLIREVKVITLKSKLVSDFRKDFQFYKVREINNYHHAHDAYLNAVVGTALIKKYPKL
ESEFVYGDYKVYDVRKMIAKSEQEIGKATAKYFFYSNIMNFFKTEITLANGEIRKRPL
IETNGETGEIVWDKGRDFATVRKVLSMPQVNIVKKTEVQTGGFSKESILPKRNSDKLI
ARKKDWDPKKYGGFDSPTVAYSVLVVAKVEKGKSKKLKSVKELLGITIMERSSFEK
NPIDFLEAKGYKEVKKDLIIKLPKYSLFELENGRKRMLASAGELQKGNELALPSKYV
NFLYLASHYEKLKGSPEDNEQKQLFVEQHKHYLDEIIEQISEFSKRVILADANLDKVL
SAYNKHRDKPIREQAENIIHLFTLTNLGAPAAFKYFDTTIDRKRYTSTKEVLDATLIHQ
SITGLYETRIDLSQLGGDKRTADGSEFEPKKKRKV
10 a.a. linker:
(SEQ ID NO: 25)
SGGSGGSGGS
4 a.a. linker:
(SEQ ID NO: 26)
SGGS
N-term NLS:
(SEQ ID NO: 27)
AAACGGACAGCCGACGGAAGCGAGTTCGAGTCACCAAAGAAGAAG
CGGAAAGTC
C-term NLS:
(SEQ ID NO: 28)
AAAAGAACCGCCGACGGCAGCGAATTCGAGCCCAAGAAGAAGAG
GAAAGTC
Cas9 BE4max:
(SEQ ID NO: 29)
GACAAGAAGTACAGCATCGGCCTGGACATCGGCACCAACTCTGTG
GGCTGGGCCGTGATCACCGACGAGTACAAGGTGCCCAGCAAGAAATTCAAGGTG
CTGGGCAACACCGACCGGCACAGCATCAAGAAGAACCTGATCGGAGCCCTGCTG
TTCGACAGCGGCGAAACAGCCGAGGCCACCCGGCTGAAGAGAACCGCCAGAAG
AAGATACACCAGACGGAAGAACCGGATCTGCTATCTGCAAGAGATCTTCAGCAA
CGAGATGGCCAAGGTGGACGACAGCTTCTTCCACAGACTGGAAGAGTCCTTCCT
GGTGGAAGAGGATAAGAAGCACGAGCGGCACCCCATCTTCGGCAACATCGTGGA
CGAGGTGGCCTACCACGAGAAGTACCCCACCATCTACCACCTGAGAAAGAAACT
GGTGGACAGCACCGACAAGGCCGACCTGCGGCTGATCTATCTGGCCCTGGCCCA
CATGATCAAGTTCCGGGGCCACTTCCTGATCGAGGGCGACCTGAACCCCGACAA
CAGCGACGTGGACAAGCTGTTCATCCAGCTGGTGCAGACCTACAACCAGCTGTTC
GAGGAAAACCCCATCAACGCCAGCGGCGTGGACGCCAAGGCCATCCTGTCTGCC
AGACTGAGCAAGAGCAGACGGCTGGAAAATCTGATCGCCCAGCTGCCCGGCGAG
AAGAAGAATGGCCTGTTCGGAAACCTGATTGCCCTGAGCCTGGGCCTGACCCCC
AACTTCAAGAGCAACTTCGACCTGGCCGAGGATGCCAAACTGCAGCTGAGCAAG
GACACCTACGACGACGACCTGGACAACCTGCTGGCCCAGATCGGCGACCAGTAC
GCCGACCTGTTTCTGGCCGCCAAGAACCTGTCCGACGCCATCCTGCTGAGCGACA
TCCTGAGAGTGAACACCGAGATCACCAAGGCCCCCCTGAGCGCCTCTATGATCA
AGAGATACGACGAGCACCACCAGGACCTGACCCTGCTGAAAGCTCTCGTGCGGC
AGCAGCTGCCTGAGAAGTACAAAGAGATTTTCTTCGACCAGAGCAAGAACGGCT
ACGCCGGCTACATTGACGGCGGAGCCAGCCAGGAAGAGTTCTACAAGTTCATCA
AGCCCATCCTGGAAAAGATGGACGGCACCGAGGAACTGCTCGTGAAGCTGAACA
GAGAGGACCTGCTGCGGAAGCAGCGGACCTTCGACAACGGCAGCATCCCCCACC
AGATCCACCTGGGAGAGCTGCACGCCATTCTGCGGCGGCAGGAAGATTTTTACCC
ATTCCTGAAGGACAACCGGGAAAAGATCGAGAAGATCCTGACCTTCCGCATCCC
CTACTACGTGGGCCCTCTGGCCAGGGGAAACAGCAGATTCGCCTGGATGACCAG
AAAGAGCGAGGAAACCATCACCCCCTGGAACTTCGAGGAAGTGGTGGACAAGG
GCGCTTCCGCCCAGAGCTTCATCGAGCGGATGACCAACTTCGATAAGAACCTGCC
CAACGAGAAGGTGCTGCCCAAGCACAGCCTGCTGTACGAGTACTTCACCGTGTAT
AACGAGCTGACCAAAGTGAAATACGTGACCGAGGGAATGAGAAAGCCCGCCTTC
CTGAGCGGCGAGCAGAAAAAGGCCATCGTGGACCTGCTGTTCAAGACCAACCGG
AAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACAT
ACCACGATCTGCTGAAAATTATCAAGGACAAGGACTTCCTGGACAATGAGGAAA
ACGAGGACATTCTGGAAGATATCGTGCTGACCCTGACACTGTTTGAGGACAGAG
AGATGATCGAGGAACGGCTGAAAACCTATGCCCACCTGTTCGACGACAAAGTGA
TGAAGCAGCTGAAGCGGCGGAGATACACCGGCTGGGGCAGGCTGAGCCGGAAG
CTGATCAACGGCATCCGGGACAAGCAGTCCGGCAAGACAATCCTGGATTTCCTG
AAGTCCGACGGCTTCGCCAACAGAAACTTCATGCAGCTGATCCACGACGACAGC
CTGACCTTTAAAGAGGACATCCAGAAAGCCCAGGTGTCCGGCCAGGGCGATAGC
CTGCACGAGCACATTGCCAATCTGGCCGGCAGCCCCGCCATTAAGAAGGGCATC
CTGCAGACAGTGAAGGTGGTGGACGAGCTCGTGAAAGTGATGGGCCGGCACAAG
CCCGAGAACATCGTGATCGAAATGGCCAGAGAGAACCAGACCACCCAGAAGGG
ACAGAAGAACAGCCGCGAGAGAATGAAGCGGATCGAAGAGGGCATCAAAGAGC
TGGGCAGCCAGATCCTGAAAGAACACCCCGTGGAAAACACCCAGCTGCAGAACG
AGAAGCTGTACCTGTACTACCTGCAGAATGGGCGGGATATGTACGTGGACCAGG
AACTGGACATCAACCGGCTGTCCGACTACGATGTGGACCATATCGTGCCTCAGAG
CTTTCTGAAGGACGACTCCATCGACAACAAGGTGCTGACCAGAAGCGACAAGAA
CCGGGGCAAGAGCGACAACGTGCCCTCCGAAGAGGTCGTGAAGAAGATGAAGA
ACTACTGGCGGCAGCTGCTGAACGCCAAGCTGATTACCCAGAGAAAGTTCGACA
ATCTGACCAAGGCCGAGAGAGGCGGCCTGAGCGAACTGGATAAGGCCGGCTTCA
TCAAGAGACAGCTGGTGGAAACCCGGCAGATCACAAAGCACGTGGCACAGATCC
TGGACTCCCGGATGAACACTAAGTACGACGAGAATGACAAGCTGATCCGGGAAG
TGAAAGTGATCACCCTGAAGTCCAAGCTGGTGTCCGATTTCCGGAAGGATTTCCA
GTTTTACAAAGTGCGCGAGATCAACAACTACCACCACGCCCACGACGCCTACCT
GAACGCCGTCGTGGGAACCGCCCTGATCAAAAAGTACCCTAAGCTGGAAAGCGA
GTTCGTGTACGGCGACTACAAGGTGTACGACGTGCGGAAGATGATCGCCAAGAG
CGAGCAGGAAATCGGCAAGGCTACCGCCAAGTACTTCTTCTACAGCAACATCAT
GAACTTTTTCAAGACCGAGATTACCCTGGCCAACGGCGAGATCCGGAAGCGGCC
TCTGATCGAGACAAACGGCGAAACCGGGGAGATCGTGTGGGATAAGGGCCGGG
ATTTTGCCACCGTGCGGAAAGTGCTGAGCATGCCCCAAGTGAATATCGTGAAAA
AGACCGAGGTGCAGACAGGCGGCTTCAGCAAAGAGTCTATCCTGCCCAAGAGGA
ACAGCGATAAGCTGATCGCCAGAAAGAAGGACTGGGACCCTAAGAAGTACGGC
GGCTTCGACAGCCCCACCGTGGCCTATTCTGTGCTGGTGGTGGCCAAAGTGGAAA
AGGGCAAGTCCAAGAAACTGAAGAGTGTGAAAGAGCTGCTGGGGATCACCATCA
TGGAAAGAAGCAGCTTCGAGAAGAATCCCATCGACTTTCTGGAAGCCAAGGGCT
ACAAAGAAGTGAAAAAGGACCTGATCATCAAGCTGCCTAAGTACTCCCTGTTCG
AGCTGGAAAACGGCCGGAAGAGAATGCTGGCCTCTGCCGGCGAACTGCAGAAGG
GAAACGAACTGGCCCTGCCCTCCAAATATGTGAACTTCCTGTACCTGGCCAGCCA
CTATGAGAAGCTGAAGGGCTCCCCCGAGGATAATGAGCAGAAACAGCTGTTTGT
GGAACAGCACAAGCACTACCTGGACGAGATCATCGAGCAGATCAGCGAGTTCTC
CAAGAGAGTGATCCTGGCCGACGCTAATCTGGACAAAGTGCTGTCCGCCTACAA
CAAGCACCGGGATAAGCCCATCAGAGAGCAGGCCGAGAATATCATCCACCTGTT
TACCCTGACCAATCTGGGAGCCCCTGCCGCCTTCAAGTACTTTGACACCACCATC
GACCGGAAGAGGTACACCAGCACCAAAGAGGTGCTGGACGCCACCCTGATCCAC
CAGAGCATCACCGGCCTGTACGAGACACGGATCGACCTGTCTCAGCTGGGAGGT
GAC
Cas9 ABEmax:
(SEQ ID NO: 30)
GACAAGAAGTACAGCATCGGCCTGGCCATCGGCACCAACTCTGTG
GGCTGGGCCGTGATCACCGACGAGTACAAGGTGCCCAGCAAGAAATTCAAGGTG
CTGGGCAACACCGACCGGCACAGCATCAAGAAGAACCTGATCGGAGCCCTGCTG
TTCGACAGCGGCGAAACAGCCGAGGCCACCCGGCTGAAGAGAACCGCCAGAAG
AAGATACACCAGACGGAAGAACCGGATCTGCTATCTGCAAGAGATCTTCAGCAA
CGAGATGGCCAAGGTGGACGACAGCTTCTTCCACAGACTGGAAGAGTCCTTCCT
GGTGGAAGAGGATAAGAAGCACGAGCGGCACCCCATCTTCGGCAACATCGTGGA
CGAGGTGGCCTACCACGAGAAGTACCCCACCATCTACCACCTGAGAAAGAAACT
GGTGGACAGCACCGACAAGGCCGACCTGCGGCTGATCTATCTGGCCCTGGCCCA
CATGATCAAGTTCCGGGGCCACTTCCTGATCGAGGGCGACCTGAACCCCGACAA
CAGCGACGTGGACAAGCTGTTCATCCAGCTGGTGCAGACCTACAACCAGCTGTTC
GAGGAAAACCCCATCAACGCCAGCGGCGTGGACGCCAAGGCCATCCTGTCTGCC
AGACTGAGCAAGAGCAGACGGCTGGAAAATCTGATCGCCCAGCTGCCCGGCGAG
AAGAAGAATGGCCTGTTCGGAAACCTGATTGCCCTGAGCCTGGGCCTGACCCCC
AACTTCAAGAGCAACTTCGACCTGGCCGAGGATGCCAAACTGCAGCTGAGCAAG
GACACCTACGACGACGACCTGGACAACCTGCTGGCCCAGATCGGCGACCAGTAC
GCCGACCTGTTTCTGGCCGCCAAGAACCTGTCCGACGCCATCCTGCTGAGCGACA
TCCTGAGAGTGAACACCGAGATCACCAAGGCCCCCCTGAGCGCCTCTATGATCA
AGAGATACGACGAGCACCACCAGGACCTGACCCTGCTGAAAGCTCTCGTGCGGC
AGCAGCTGCCTGAGAAGTACAAAGAGATTTTCTTCGACCAGAGCAAGAACGGCT
ACGCCGGCTACATTGACGGCGGAGCCAGCCAGGAAGAGTTCTACAAGTTCATCA
AGCCCATCCTGGAAAAGATGGACGGCACCGAGGAACTGCTCGTGAAGCTGAACA
GAGAGGACCTGCTGCGGAAGCAGCGGACCTTCGACAACGGCAGCATCCCCCACC
AGATCCACCTGGGAGAGCTGCACGCCATTCTGCGGCGGCAGGAAGATTTTTACCC
ATTCCTGAAGGACAACCGGGAAAAGATCGAGAAGATCCTGACCTTCCGCATCCC
CTACTACGTGGGCCCTCTGGCCAGGGGAAACAGCAGATTCGCCTGGATGACCAG
AAAGAGCGAGGAAACCATCACCCCCTGGAACTTCGAGGAAGTGGTGGACAAGG
GCGCTTCCGCCCAGAGCTTCATCGAGCGGATGACCAACTTCGATAAGAACCTGCC
CAACGAGAAGGTGCTGCCCAAGCACAGCCTGCTGTACGAGTACTTCACCGTGTAT
AACGAGCTGACCAAAGTGAAATACGTGACCGAGGGAATGAGAAAGCCCGCCTTC
CTGAGCGGCGAGCAGAAAAAGGCCATCGTGGACCTGCTGTTCAAGACCAACCGG
AAAGTGACCGTGAAGCAGCTGAAAGAGGACTACTTCAAGAAAATCGAGTGCTTC
GACTCCGTGGAAATCTCCGGCGTGGAAGATCGGTTCAACGCCTCCCTGGGCACAT
ACCACGATCTGCTGAAAATTATCAAGGACAAGGACTTCCTGGACAATGAGGAAA
ACGAGGACATTCTGGAAGATATCGTGCTGACCCTGACACTGTTTGAGGACAGAG
AGATGATCGAGGAACGGCTGAAAACCTATGCCCACCTGTTCGACGACAAAGTGA
TGAAGCAGCTGAAGCGGCGGAGATACACCGGCTGGGGCAGGCTGAGCCGGAAG
CTGATCAACGGCATCCGGGACAAGCAGTCCGGCAAGACAATCCTGGATTTCCTG
AAGTCCGACGGCTTCGCCAACAGAAACTTCATGCAGCTGATCCACGACGACAGC
CTGACCTTTAAAGAGGACATCCAGAAAGCCCAGGTGTCCGGCCAGGGCGATAGC
CTGCACGAGCACATTGCCAATCTGGCCGGCAGCCCCGCCATTAAGAAGGGCATC
CTGCAGACAGTGAAGGTGGTGGACGAGCTCGTGAAAGTGATGGGCCGGCACAAG
CCCGAGAACATCGTGATCGAAATGGCCAGAGAGAACCAGACCACCCAGAAGGG
ACAGAAGAACAGCCGCGAGAGAATGAAGCGGATCGAAGAGGGCATCAAAGAGC
TGGGCAGCCAGATCCTGAAAGAACACCCCGTGGAAAACACCCAGCTGCAGAACG
AGAAGCTGTACCTGTACTACCTGCAGAATGGGCGGGATATGTACGTGGACCAGG
AACTGGACATCAACCGGCTGTCCGACTACGATGTGGACCATATCGTGCCTCAGAG
CTTTCTGAAGGACGACTCCATCGACAACAAGGTGCTGACCAGAAGCGACAAGAA
CCGGGGCAAGAGCGACAACGTGCCCTCCGAAGAGGTCGTGAAGAAGATGAAGA
ACTACTGGCGGCAGCTGCTGAACGCCAAGCTGATTACCCAGAGAAAGTTCGACA
ATCTGACCAAGGCCGAGAGAGGCGGCCTGAGCGAACTGGATAAGGCCGGCTTCA
TCAAGAGACAGCTGGTGGAAACCCGGCAGATCACAAAGCACGTGGCACAGATCC
TGGACTCCCGGATGAACACTAAGTACGACGAGAATGACAAGCTGATCCGGGAAG
TGAAAGTGATCACCCTGAAGTCCAAGCTGGTGTCCGATTTCCGGAAGGATTTCCA
GTTTTACAAAGTGCGCGAGATCAACAACTACCACCACGCCCACGACGCCTACCT
GAACGCCGTCGTGGGAACCGCCCTGATCAAAAAGTACCCTAAGCTGGAAAGCGA
GTTCGTGTACGGCGACTACAAGGTGTACGACGTGCGGAAGATGATCGCCAAGAG
CGAGCAGGAAATCGGCAAGGCTACCGCCAAGTACTTCTTCTACAGCAACATCAT
GAACTTTTTCAAGACCGAGATTACCCTGGCCAACGGCGAGATCCGGAAGCGGCC
TCTGATCGAGACAAACGGCGAAACCGGGGAGATCGTGTGGGATAAGGGCCGGG
ATTTTGCCACCGTGCGGAAAGTGCTGAGCATGCCCCAAGTGAATATCGTGAAAA
AGACCGAGGTGCAGACAGGCGGCTTCAGCAAAGAGTCTATCCTGCCCAAGAGGA
ACAGCGATAAGCTGATCGCCAGAAAGAAGGACTGGGACCCTAAGAAGTACGGC
GGCTTCGACAGCCCCACCGTGGCCTATTCTGTGCTGGTGGTGGCCAAAGTGGAAA
AGGGCAAGTCCAAGAAACTGAAGAGTGTGAAAGAGCTGCTGGGGATCACCATCA
TGGAAAGAAGCAGCTTCGAGAAGAATCCCATCGACTTTCTGGAAGCCAAGGGCT
ACAAAGAAGTGAAAAAGGACCTGATCATCAAGCTGCCTAAGTACTCCCTGTTCG
AGCTGGAAAACGGCCGGAAGAGAATGCTGGCCTCTGCCGGCGAACTGCAGAAGG
GAAACGAACTGGCCCTGCCCTCCAAATATGTGAACTTCCTGTACCTGGCCAGCCA
CTATGAGAAGCTGAAGGGCTCCCCCGAGGATAATGAGCAGAAACAGCTGTTTGT
GGAACAGCACAAGCACTACCTGGACGAGATCATCGAGCAGATCAGCGAGTTCTC
CAAGAGAGTGATCCTGGCCGACGCTAATCTGGACAAAGTGCTGTCCGCCTACAA
CAAGCACCGGGATAAGCCCATCAGAGAGCAGGCCGAGAATATCATCCACCTGTT
TACCCTGACCAATCTGGGAGCCCCTGCCGCCTTCAAGTACTTTGACACCACCATC
GACCGGAAGAGGTACACCAGCACCAAAGAGGTGCTGGACGCCACCCTGATCCAC
CAGAGCATCACCGGCCTGTACGAGACACGGATCGACCTGTCTCAGCTGGGAGGT
GAC
BE4max XTEN linker:
(SEQ ID NO: 31)
TCTGGAGGATCTAGCGGAGGATCCTCTGGCAGCGAGACACCAGGA
ACAAGCGAGTCAGCAACACCAGAGAGCAGTGGCGGCAGCAGCGGCGGCAGC
ABEmax XTEN linker first:
(SEQ ID NO: 32)
TCTGGAGGATCTAGCGGAGGATCCTCTGGAAGCGAGACACCAGGC
ACAAGCGAGTCCGCCACACCAGAGAGCTCCGGCGGCTCCTCCGGAGGATCC
ABEmax XTEN linker second:
(SEQ ID NO: 33)
TCCGGAGGATCTAGCGGAGGCTCCTCTGGCTCTGAGACACCTGGCA
CAAGCGAGAGCGCAACACCTGAAAGCAGCGGGGGCAGCAGCGGGGGGTCA
BE4max 10 a.a. linker 1:
(SEQ ID NO: 34)
AGCGGCGGGAGCGGCGGGAGCGGGGGGAGC
BE4max 10 a.a. linker 2:
(SEQ ID NO: 35)
AGCGGAGGATCCGGAGGATCTGGAGGCAGC
BE4max 4 a.a. linker:
(SEQ ID NO: 36)
TCTGGCGGCTCA
Rat APOBEC1:
(SEQ ID NO: 37)
TCCTCAGAGACTGGGCCTGTCGCCGTCGATCCAACCCTGCGCCGCC
GGATTGAACCTCACGAGTTTGAAGTGTTCTTTGACCCCCGGGAGCTGAGAAAGG
AGACATGCCTGCTGTACGAGATCAACTGGGGAGGCAGGCACTCCATCTGGAGGC
ACACCTCTCAGAACACAAATAAGCACGTGGAGGTGAACTTCATCGAGAAGTTTA
CCACAGAGCGGTACTTCTGCCCCAATACCAGATGTAGCATCACATGGTTTCTGAG
CTGGTCCCCTTGCGGAGAGTGTAGCAGGGCCATCACCGAGTTCCTGTCCAGATAT
CCACACGTGACACTGTTTATCTACATCGCCAGGCTGTATCACCACGCAGACCCAA
GGAATAGGCAGGGCCTGCGCGATCTGATCAGCTCCGGCGTGACCATCCAGATCA
TGACAGAGCAGGAGTCCGGCTACTGCTGGCGGAACTTCGTGAATTATTCTCCTAG
CAACGAGGCCCACTGGCCTAGGTACCCACACCTGTGGGTGCGCCTGTACGTGCTG
GAGCTGTATTGCATCATCCTGGGCCTGCCCCCTTGTCTGAATATCCTGCGGAGAA
AGCAGCCCCAGCTGACCTTCTTTACAATCGCCCTGCAGTCTTGTCACTATCAGAG
GCTGCCACCCCACATCCTGTGGGCCACAGGCCTGAAG
Anc689 APOBEC:
(SEQ ID NO: 38)
AGCAGTGAAACCGGACCAGTGGCAGTGGACCCAACCCTGAGGAGA
CGGATTGAGCCCCATGAATTTGAAGTGTTCTTTGACCCAAGGGAGCTGAGGAAG
GAGACATGCCTGCTGTACGAGATCAAGTGGGGCACAAGCCACAAGATCTGGCGC
CACAGCTCCAAGAACACCACAAAGCACGTGGAAGTGAATTTCATCGAGAAGTTT
ACCTCCGAGCGGCACTTCTGCCCCTCTACCAGCTGTTCCATCACATGGTTTCTGTC
TTGGAGCCCTTGCGGCGAGTGTTCCAAGGCCATCACCGAGTTCCTGTCTCAGCAC
CCTAACGTGACCCTGGTCATCTACGTGGCCCGGCTGTATCACCACATGGACCAGC
AGAACAGGCAGGGCCTGCGCGATCTGGTGAATTCTGGCGTGACCATCCAGATCA
TGACAGCCCCAGAGTACGACTATTGCTGGCGGAACTTCGTGAATTATCCACCTGG
CAAGGAGGCACACTGGCCAAGATACCCACCCCTGTGGATGAAGCTGTATGCACT
GGAGCTGCACGCAGGAATCCTGGGCCTGCCTCCATGTCTGAATATCCTGCGGAGA
AAGCAGCCCCAGCTGACATTTTTCACCATTGCTCTGCAGTCTTGTCACTATCAGC
GGCTGCCTCCTCATATTCTGTGGGCTACAGGCCTTAAA
Anc687 APOBEC:
(SEQ ID NO: 39)
TCATCAGAAACAGGACCAGTCGCCGTGGACCCAACACTGAGGAGA
AGGATTGAGCCCCATGAATTTGAAGTCTTTTTCGACCCCAGGGAGCTGAGGAAG
GAGGCATGCCTGCTGTACGAGATCAAGTGGGGCACAAGCCACAAGATCTGGCGC
AACAGCGGCAAGAACACCACAAAGCACGTGGAAGTGAATTTCATCGAGAAGTTT
ACCTCCGAGCGGCACTTCTGCCCCTCTATCAGCTGTTCCATCACATGGTTTCTGTC
TTGGAGCCCTTGCTGGGAGTGTTCCAAGGCCATCCGCGAGTTCCTGTCTCAGCAC
CCTAACGTGACCCTGGTCATCTACGTGGCCCGGCTGTTTCAACACATGGACCAGC
AGAACAGGCAGGGCCTGCGCGATCTGGTGAATTCTGGCGTGACCATCCAGATCA
TGACAGCCTCAGAGTACGACCATTGCTGGCGGAACTTCGTGAATTATCCACCTGG
CAAGGAGGCACACTGGCCAAGATACCCACCCCTGTGGATGAAGCTGTATGCACT
GGAGCTGCACGCAGGAATCCTGGGCCTGCCTCCATGTCTGAATATCCTGCGGAGA
AAGCAGCCCCAGCTGACATTTTTCACTATCGCACTGCAGAGCTGTCATTACCAGA
GACTGCCTCCTCATATCCTGTGGGCTACAGGCCTTAAA
Anc686 APOBEC:
(SEQ ID NO: 40)
AGCAGCGAGACAGGACCCGTGGCAGTGGACCCTACACTGAGGAGG
AGGATTGAGCCCGAATTTTTCAACAGGAACTACGACCCCAGAGAGCTGCGGAAG
GAGACATACCTGCTGTATGAGATCAAGTGGGGCAAGGAGTCCAAGATCTGGCGG
CACACCTCTAACAATAGAACACAGCACGCCGAGGTGAACTTCCTGGAGAACTTC
TTTAATGAGCTGTACTTTAATCCTTCTACCCACTGCAGCATCACATGGTTCCTGAG
CTGGTCCCCATGCGGCGAGTGTTCTAAGGCCATCGTGGAGTTTCTGAAGGAGCAC
CCCAACGTGAATCTGGAGATCTACGTGGCCAGGCTGTATCTGTGCGAGGACGAG
AGGAACAGGCAGGGCCTGCGGGATCTGGTGAATAGCGGCGTGACCATCAGAATC
ATGAACCTGCCTGACTACAATTATTGTTGGCGCACATTCGTGTCCCACCAGGGAG
GCGACGAGGATTATTGGCCAAGGCACTTTGCACCATGGGTGCGCCTGTACGTGCT
GGAGCTGTATTGCATCATCCTGGGCCTGCCCCCTTGTCTGAACATCCTGCGGAGA
AAGCAGCCCCAGCTGACATTCTTCACCATCGCACTGCAGAGTTGTCATTACCAGC
GACTGCCTCCTCATATCCTGTGGGCTACAGGCCTTAAA
Anc655 APOBEC:
(SEQ ID NO: 41)
TCATCAGAGACCGGACCTGTGGCAGTGGACCCAACCCTGCGACGG
AGAATCGAGCCCTTTTACTTTCAGTTCAACAACGACCCAAGAGCCTGCCGGAGAA
AGACCTACCTGTGCTATGAGCTGAAGCAGGACGGCTCTACCTGGGTGTGGAAGC
GGACACTGCACAACAAGGGCAGACACGCCGAGATCTGCTTCCTGGAGAAGATCA
GCTCCCTGGAGAAGCTGGACCCTGCCCAGCACTACAGGATCACATGGTATATGTC
TTGGAGCCCATGCTCCAACTGTGCCCAGAAGATCGTGGATTTTCTGAAGGAGCAC
CCACACGTGAATCTGCGGATCTACGTGGCCAGACTGTACTATCACGAGGAGGAG
AGGTATCAGGAGGGCCTGAGGAACCTGAGGCGCTCCGGCGTGTCTATCAGAGTG
ATGGACCTGCCCGATTTCGAGCACTGCTGGGAGACATTCGTGGATAACGGAGGA
GGACCTTTCCAGCCATGGCCCGGCCTGGAGGAGCTGAATAGCAAGCAGCTGTCC
CGGAGACTGCAGGCAGGAATCCTGGGCCTGCCCCCTTGTCTGAATATCCTGAGGC
GCAAGCAGCCCCAGCTGACATTTTTCACCATCGCACTGCAGAGTTGTCATTATCA
GCGACTGCCTCCTCATATCCTGTGGGCTACAGGCCTTAAA
Anc733 APOBEC:
(SEQ ID NO: 42)
AGCAGCGAGACCGGACCTGTGGCAGTGGACCCAACCCTGAGAAGA
CGCATTGAGCCATTTCATTTTCAGTTTAACAACGACCCCAGAGCCTACCGGAGAA
AGACCTACCTGTGCTATGAGCTGAAGCAGGACGGCTCCACCTGGGTGCTGGATC
GGACACTGAGAAACAAGGGCCGGCACGCCGAGATCTGTTTCCTGGACAAGATCA
ATTCCTGGGAGAGGCTGGATCCCGCCCAGCACTACCGCGTGACATGGTATATGA
GCTGGTCCCCTTGCTCTAACTGTGCCCAGCAGGTGGTGGATTTCCTGAAGGAGCA
CCCACACGTGAATCTGCGGATCTTTGCCGCCAGACTGTACTATCACGAGCAGAGG
CGCTATCAGGAGGGCCTGCGGAGCCTGAGGGGAAGCGGAGTGCCTGTGGCCGTG
ATGACCCTGCCAGACTTCGAGCACTGCTGGGAGACATTTGTGGATCACGGCGGCC
GGCCATTCCAGCCATGGGACGGCCTGGAGGAGCTGAACTCTAGGAGCCTGTCCC
GGAGACTGCAGGCAGGAATCCTGGGCCTGCCCCCTTGTCTGAATATCCTGAGGCG
CAAGCAGCCCCAGCTGACCTTTTTTACCATCGCACTGCAGAGTTGTCACTACCAG
AGACTGCCTCCTCATATCCTGTGGGCTACAGGCCTTAAA
TadA WT:
(SEQ ID NO: 43)
TCTGAAGTCGAGTTTAGCCACGAGTATTGGATGAGGCACGCACTGA
CCCTGGCAAAGCGAGCATGGGATGAAAGAGAAGTCCCCGTGGGCGCCGTGCTGG
TGCACAACAATAGAGTGATCGGAGAGGGATGGAACAGGCCAATCGGCCGCCACG
ACCCTACCGCACACGCAGAGATCATGGCACTGAGGCAGGGAGGCCTGGTCATGC
AGAATTACCGCCTGATCGATGCCACCCTGTATGTGACACTGGAGCCATGCGTGAT
GTGCGCAGGAGCAATGATCCACAGCAGGATCGGAAGAGTGGTGTTCGGAGCACG
GGACGCCAAGACCGGCGCAGCAGGCTCCCTGATGGATGTGCTGCACCACCCCGG
CATGAACCACCGGGTGGAGATCACAGAGGGAATCCTGGCAGACGAGTGCGCCGC
CCTGCTGAGCGATTTCTTTAGAATGCGGAGACAGGAGATCAAGGCCCAGAAGAA
GGCACAGAGCTCCACCGAC
TadA 7.10:
(SEQ ID NO: 44)
TCTGAGGTGGAGTTTTCCCACGAGTACTGGATGAGACATGCCCTGA
CCCTGGCCAAGAGGGCACGCGATGAGAGGGAGGTGCCTGTGGGAGCCGTGCTGG
TGCTGAACAATAGAGTGATCGGCGAGGGCTGGAACAGAGCCATCGGCCTGCACG
ACCCAACAGCCCATGCCGAAATTATGGCCCTGAGACAGGGCGGCCTGGTCATGC
AGAACTACAGACTGATTGACGCCACCCTGTACGTGACATTCGAGCCTTGCGTGAT
GTGCGCCGGCGCCATGATCCACTCTAGGATCGGCCGCGTGGTGTTTGGCGTGAGG
AACGCAAAAACCGGCGCCGCAGGCTCCCTGATGGACGTGCTGCACTACCCCGGC
ATGAATCACCGCGTCGAAATTACCGAGGGAATCCTGGCAGATGAATGTGCCGCC
CTGCTGTGCTATTTCTTTCGGATGCCTAGACAGGTGTTCAATGCTCAGAAGAAGG
CCCAGAGCTCCACCGAC
BE4max:
(SEQ ID NO: 45)
ATGAAACGGACAGCCGACGGAAGCGAGTTCGAGTCACCAAAGAAG
AAGCGGAAAGTCTCCTCAGAGACTGGGCCTGTCGCCGTCGATCCAACCCTGCGC
CGCCGGATTGAACCTCACGAGTTTGAAGTGTTCTTTGACCCCCGGGAGCTGAGAA
AGGAGACATGCCTGCTGTACGAGATCAACTGGGGAGGCAGGCACTCCATCTGGA
GGCACACCTCTCAGAACACAAATAAGCACGTGGAGGTGAACTTCATCGAGAAGT
TTACCACAGAGCGGTACTTCTGCCCCAATACCAGATGTAGCATCACATGGTTTCT
GAGCTGGTCCCCTTGCGGAGAGTGTAGCAGGGCCATCACCGAGTTCCTGTCCAGA
TATCCACACGTGACACTGTTTATCTACATCGCCAGGCTGTATCACCACGCAGACC
CAAGGAATAGGCAGGGCCTGCGCGATCTGATCAGCTCCGGCGTGACCATCCAGA
TCATGACAGAGCAGGAGTCCGGCTACTGCTGGCGGAACTTCGTGAATTATTCTCC
TAGCAACGAGGCCCACTGGCCTAGGTACCCACACCTGTGGGTGCGCCTGTACGT
GCTGGAGCTGTATTGCATCATCCTGGGCCTGCCCCCTTGTCTGAATATCCTGCGG
AGAAAGCAGCCCCAGCTGACCTTCTTTACAATCGCCCTGCAGTCTTGTCACTATC
AGAGGCTGCCACCCCACATCCTGTGGGCCACAGGCCTGAAGTCTGGAGGATCTA
GCGGAGGATCCTCTGGCAGCGAGACACCAGGAACAAGCGAGTCAGCAACACCA
GAGAGCAGTGGCGGCAGCAGCGGCGGCAGCGACAAGAAGTACAGCATCGGCCT
GGACATCGGCACCAACTCTGTGGGCTGGGCCGTGATCACCGACGAGTACAAGGT
GCCCAGCAAGAAATTCAAGGTGCTGGGCAACACCGACCGGCACAGCATCAAGAA
GAACCTGATCGGAGCCCTGCTGTTCGACAGCGGCGAAACAGCCGAGGCCACCCG
GCTGAAGAGAACCGCCAGAAGAAGATACACCAGACGGAAGAACCGGATCTGCT
ATCTGCAAGAGATCTTCAGCAACGAGATGGCCAAGGTGGACGACAGCTTCTTCC
ACAGACTGGAAGAGTCCTTCCTGGTGGAAGAGGATAAGAAGCACGAGCGGCACC
CCATCTTCGGCAACATCGTGGACGAGGTGGCCTACCACGAGAAGTACCCCACCA
TCTACCACCTGAGAAAGAAACTGGTGGACAGCACCGACAAGGCCGACCTGCGGC
TGATCTATCTGGCCCTGGCCCACATGATCAAGTTCCGGGGCCACTTCCTGATCGA
GGGCGACCTGAACCCCGACAACAGCGACGTGGACAAGCTGTTCATCCAGCTGGT
GCAGACCTACAACCAGCTGTTCGAGGAAAACCCCATCAACGCCAGCGGCGTGGA
CGCCAAGGCCATCCTGTCTGCCAGACTGAGCAAGAGCAGACGGCTGGAAAATCT
GATCGCCCAGCTGCCCGGCGAGAAGAAGAATGGCCTGTTCGGAAACCTGATTGC
CCTGAGCCTGGGCCTGACCCCCAACTTCAAGAGCAACTTCGACCTGGCCGAGGA
TGCCAAACTGCAGCTGAGCAAGGACACCTACGACGACGACCTGGACAACCTGCT
GGCCCAGATCGGCGACCAGTACGCCGACCTGTTTCTGGCCGCCAAGAACCTGTCC
GACGCCATCCTGCTGAGCGACATCCTGAGAGTGAACACCGAGATCACCAAGGCC
CCCCTGAGCGCCTCTATGATCAAGAGATACGACGAGCACCACCAGGACCTGACC
CTGCTGAAAGCTCTCGTGCGGCAGCAGCTGCCTGAGAAGTACAAAGAGATTTTCT
TCGACCAGAGCAAGAACGGCTACGCCGGCTACATTGACGGCGGAGCCAGCCAGG
AAGAGTTCTACAAGTTCATCAAGCCCATCCTGGAAAAGATGGACGGCACCGAGG
AACTGCTCGTGAAGCTGAACAGAGAGGACCTGCTGCGGAAGCAGCGGACCTTCG
ACAACGGCAGCATCCCCCACCAGATCCACCTGGGAGAGCTGCACGCCATTCTGC
GGCGGCAGGAAGATTTTTACCCATTCCTGAAGGACAACCGGGAAAAGATCGAGA
AGATCCTGACCTTCCGCATCCCCTACTACGTGGGCCCTCTGGCCAGGGGAAACAG
CAGATTCGCCTGGATGACCAGAAAGAGCGAGGAAACCATCACCCCCTGGAACTT
CGAGGAAGTGGTGGACAAGGGCGCTTCCGCCCAGAGCTTCATCGAGCGGATGAC
CAACTTCGATAAGAACCTGCCCAACGAGAAGGTGCTGCCCAAGCACAGCCTGCT
GTACGAGTACTTCACCGTGTATAACGAGCTGACCAAAGTGAAATACGTGACCGA
GGGAATGAGAAAGCCCGCCTTCCTGAGCGGCGAGCAGAAAAAGGCCATCGTGGA
CCTGCTGTTCAAGACCAACCGGAAAGTGACCGTGAAGCAGCTGAAAGAGGACTA
CTTCAAGAAAATCGAGTGCTTCGACTCCGTGGAAATCTCCGGCGTGGAAGATCG
GTTCAACGCCTCCCTGGGCACATACCACGATCTGCTGAAAATTATCAAGGACAAG
GACTTCCTGGACAATGAGGAAAACGAGGACATTCTGGAAGATATCGTGCTGACC
CTGACACTGTTTGAGGACAGAGAGATGATCGAGGAACGGCTGAAAACCTATGCC
CACCTGTTCGACGACAAAGTGATGAAGCAGCTGAAGCGGCGGAGATACACCGGC
TGGGGCAGGCTGAGCCGGAAGCTGATCAACGGCATCCGGGACAAGCAGTCCGGC
AAGACAATCCTGGATTTCCTGAAGTCCGACGGCTTCGCCAACAGAAACTTCATGC
AGCTGATCCACGACGACAGCCTGACCTTTAAAGAGGACATCCAGAAAGCCCAGG
TGTCCGGCCAGGGCGATAGCCTGCACGAGCACATTGCCAATCTGGCCGGCAGCC
CCGCCATTAAGAAGGGCATCCTGCAGACAGTGAAGGTGGTGGACGAGCTCGTGA
AAGTGATGGGCCGGCACAAGCCCGAGAACATCGTGATCGAAATGGCCAGAGAG
AACCAGACCACCCAGAAGGGACAGAAGAACAGCCGCGAGAGAATGAAGCGGAT
CGAAGAGGGCATCAAAGAGCTGGGCAGCCAGATCCTGAAAGAACACCCCGTGG
AAAACACCCAGCTGCAGAACGAGAAGCTGTACCTGTACTACCTGCAGAATGGGC
GGGATATGTACGTGGACCAGGAACTGGACATCAACCGGCTGTCCGACTACGATG
TGGACCATATCGTGCCTCAGAGCTTTCTGAAGGACGACTCCATCGACAACAAGGT
GCTGACCAGAAGCGACAAGAACCGGGGCAAGAGCGACAACGTGCCCTCCGAAG
AGGTCGTGAAGAAGATGAAGAACTACTGGCGGCAGCTGCTGAACGCCAAGCTGA
TTACCCAGAGAAAGTTCGACAATCTGACCAAGGCCGAGAGAGGCGGCCTGAGCG
AACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGA
ATGACAAGCTGATCCGGGAAGTGAAAGTGATCACCCTGAAGTCCAAGCTGGTGT
CCGATTTCCGGAAGGATTTCCAGTTTTACAAAGTGCGCGAGATCAACAACTACCA
CCACGCCCACGACGCCTACCTGAACGCCGTCGTGGGAACCGCCCTGATCAAAAA
GTACCCTAAGCTGGAAAGCGAGTTCGTGTACGGCGACTACAAGGTGTACGACGT
GCGGAAGATGATCGCCAAGAGCGAGCAGGAAATCGGCAAGGCTACCGCCAAGT
ACTTCTTCTACAGCAACATCATGAACTTTTTCAAGACCGAGATTACCCTGGCCAA
CGGCGAGATCCGGAAGCGGCCTCTGATCGAGACAAACGGCGAAACCGGGGAGA
TCGTGTGGGATAAGGGCCGGGATTTTGCCACCGTGCGGAAAGTGCTGAGCATGC
CCCAAGTGAATATCGTGAAAAAGACCGAGGTGCAGACAGGCGGCTTCAGCAAAG
AGTCTATCCTGCCCAAGAGGAACAGCGATAAGCTGATCGCCAGAAAGAAGGACT
GGGACCCTAAGAAGTACGGCGGCTTCGACAGCCCCACCGTGGCCTATTCTGTGCT
GGTGGTGGCCAAAGTGGAAAAGGGCAAGTCCAAGAAACTGAAGAGTGTGAAAG
AGCTGCTGGGGATCACCATCATGGAAAGAAGCAGCTTCGAGAAGAATCCCATCG
ACTTTCTGGAAGCCAAGGGCTACAAAGAAGTGAAAAAGGACCTGATCATCAAGC
TGCCTAAGTACTCCCTGTTCGAGCTGGAAAACGGCCGGAAGAGAATGCTGGCCT
CTGCCGGCGAACTGCAGAAGGGAAACGAACTGGCCCTGCCCTCCAAATATGTGA
ACTTCCTGTACCTGGCCAGCCACTATGAGAAGCTGAAGGGCTCCCCCGAGGATA
ATGAGCAGAAACAGCTGTTTGTGGAACAGCACAAGCACTACCTGGACGAGATCA
TCGAGCAGATCAGCGAGTTCTCCAAGAGAGTGATCCTGGCCGACGCTAATCTGG
ACAAAGTGCTGTCCGCCTACAACAAGCACCGGGATAAGCCCATCAGAGAGCAGG
CCGAGAATATCATCCACCTGTTTACCCTGACCAATCTGGGAGCCCCTGCCGCCTT
CAAGTACTTTGACACCACCATCGACCGGAAGAGGTACACCAGCACCAAAGAGGT
GCTGGACGCCACCCTGATCCACCAGAGCATCACCGGCCTGTACGAGACACGGAT
CGACCTGTCTCAGCTGGGAGGTGACAGCGGCGGGAGCGGCGGGAGCGGGGGGA
GCACTAATCTGAGCGACATCATTGAGAAGGAGACTGGGAAACAGCTGGTCATTC
AGGAGTCCATCCTGATGCTGCCTGAGGAGGTGGAGGAAGTGATCGGCAACAAGC
CAGAGTCTGACATCCTGGTGCACACCGCCTACGACGAGTCCACAGATGAGAATG
TGATGCTGCTGACCTCTGACGCCCCCGAGTATAAGCCTTGGGCCCTGGTCATCCA
GGATTCTAACGGCGAGAATAAGATCAAGATGCTGAGCGGAGGATCCGGAGGATC
TGGAGGCAGCACCAACCTGTCTGACATCATCGAGAAGGAGACAGGCAAGCAGCT
GGTCATCCAGGAGAGCATCCTGATGCTGCCCGAAGAAGTCGAAGAAGTGATCGG
AAACAAGCCTGAGAGCGATATCCTGGTCCATACCGCCTACGACGAGAGTACCGA
CGAAAATGTGATGCTGCTGACATCCGACGCCCCAGAGTATAAGCCCTGGGCTCTG
GTCATCCAGGATTCCAACGGAGAGAACAAAATCAAAATGCTGTCTGGCGGCTCA
AAAAGAACCGCCGACGGCAGCGAATTCGAGCCCAAGAAGAAGAGGAAAGTC
AncBE4max689:
(SEQ ID NO: 46)
ATGAAACGGACAGCCGACGGAAGCGAGTTCGAGTCACCAAAGAAG
AAGCGGAAAGTCAGCAGTGAAACCGGACCAGTGGCAGTGGACCCAACCCTGAG
GAGACGGATTGAGCCCCATGAATTTGAAGTGTTCTTTGACCCAAGGGAGCTGAG
GAAGGAGACATGCCTGCTGTACGAGATCAAGTGGGGCACAAGCCACAAGATCTG
GCGCCACAGCTCCAAGAACACCACAAAGCACGTGGAAGTGAATTTCATCGAGAA
GTTTACCTCCGAGCGGCACTTCTGCCCCTCTACCAGCTGTTCCATCACATGGTTTC
TGTCTTGGAGCCCTTGCGGCGAGTGTTCCAAGGCCATCACCGAGTTCCTGTCTCA
GCACCCTAACGTGACCCTGGTCATCTACGTGGCCCGGCTGTATCACCACATGGAC
CAGCAGAACAGGCAGGGCCTGCGCGATCTGGTGAATTCTGGCGTGACCATCCAG
ATCATGACAGCCCCAGAGTACGACTATTGCTGGCGGAACTTCGTGAATTATCCAC
CTGGCAAGGAGGCACACTGGCCAAGATACCCACCCCTGTGGATGAAGCTGTATG
CACTGGAGCTGCACGCAGGAATCCTGGGCCTGCCTCCATGTCTGAATATCCTGCG
GAGAAAGCAGCCCCAGCTGACATTTTTCACCATTGCTCTGCAGTCTTGTCACTAT
CAGCGGCTGCCTCCTCATATTCTGTGGGCTACAGGCCTTAAATCTGGAGGATCTA
GCGGAGGATCCTCTGGCAGCGAGACACCAGGAACAAGCGAGTCAGCAACACCA
GAGAGCAGTGGCGGCAGCAGCGGCGGCAGCGACAAGAAGTACAGCATCGGCCT
GGACATCGGCACCAACTCTGTGGGCTGGGCCGTGATCACCGACGAGTACAAGGT
GCCCAGCAAGAAATTCAAGGTGCTGGGCAACACCGACCGGCACAGCATCAAGAA
GAACCTGATCGGAGCCCTGCTGTTCGACAGCGGCGAAACAGCCGAGGCCACCCG
GCTGAAGAGAACCGCCAGAAGAAGATACACCAGACGGAAGAACCGGATCTGCT
ATCTGCAAGAGATCTTCAGCAACGAGATGGCCAAGGTGGACGACAGCTTCTTCC
ACAGACTGGAAGAGTCCTTCCTGGTGGAAGAGGATAAGAAGCACGAGCGGCACC
CCATCTTCGGCAACATCGTGGACGAGGTGGCCTACCACGAGAAGTACCCCACCA
TCTACCACCTGAGAAAGAAACTGGTGGACAGCACCGACAAGGCCGACCTGCGGC
TGATCTATCTGGCCCTGGCCCACATGATCAAGTTCCGGGGCCACTTCCTGATCGA
GGGCGACCTGAACCCCGACAACAGCGACGTGGACAAGCTGTTCATCCAGCTGGT
GCAGACCTACAACCAGCTGTTCGAGGAAAACCCCATCAACGCCAGCGGCGTGGA
CGCCAAGGCCATCCTGTCTGCCAGACTGAGCAAGAGCAGACGGCTGGAAAATCT
GATCGCCCAGCTGCCCGGCGAGAAGAAGAATGGCCTGTTCGGAAACCTGATTGC
CCTGAGCCTGGGCCTGACCCCCAACTTCAAGAGCAACTTCGACCTGGCCGAGGA
TGCCAAACTGCAGCTGAGCAAGGACACCTACGACGACGACCTGGACAACCTGCT
GGCCCAGATCGGCGACCAGTACGCCGACCTGTTTCTGGCCGCCAAGAACCTGTCC
GACGCCATCCTGCTGAGCGACATCCTGAGAGTGAACACCGAGATCACCAAGGCC
CCCCTGAGCGCCTCTATGATCAAGAGATACGACGAGCACCACCAGGACCTGACC
CTGCTGAAAGCTCTCGTGCGGCAGCAGCTGCCTGAGAAGTACAAAGAGATTTTCT
TCGACCAGAGCAAGAACGGCTACGCCGGCTACATTGACGGCGGAGCCAGCCAGG
AAGAGTTCTACAAGTTCATCAAGCCCATCCTGGAAAAGATGGACGGCACCGAGG
AACTGCTCGTGAAGCTGAACAGAGAGGACCTGCTGCGGAAGCAGCGGACCTTCG
ACAACGGCAGCATCCCCCACCAGATCCACCTGGGAGAGCTGCACGCCATTCTGC
GGCGGCAGGAAGATTTTTACCCATTCCTGAAGGACAACCGGGAAAAGATCGAGA
AGATCCTGACCTTCCGCATCCCCTACTACGTGGGCCCTCTGGCCAGGGGAAACAG
CAGATTCGCCTGGATGACCAGAAAGAGCGAGGAAACCATCACCCCCTGGAACTT
CGAGGAAGTGGTGGACAAGGGCGCTTCCGCCCAGAGCTTCATCGAGCGGATGAC
CAACTTCGATAAGAACCTGCCCAACGAGAAGGTGCTGCCCAAGCACAGCCTGCT
GTACGAGTACTTCACCGTGTATAACGAGCTGACCAAAGTGAAATACGTGACCGA
GGGAATGAGAAAGCCCGCCTTCCTGAGCGGCGAGCAGAAAAAGGCCATCGTGGA
CCTGCTGTTCAAGACCAACCGGAAAGTGACCGTGAAGCAGCTGAAAGAGGACTA
CTTCAAGAAAATCGAGTGCTTCGACTCCGTGGAAATCTCCGGCGTGGAAGATCG
GTTCAACGCCTCCCTGGGCACATACCACGATCTGCTGAAAATTATCAAGGACAAG
GACTTCCTGGACAATGAGGAAAACGAGGACATTCTGGAAGATATCGTGCTGACC
CTGACACTGTTTGAGGACAGAGAGATGATCGAGGAACGGCTGAAAACCTATGCC
CACCTGTTCGACGACAAAGTGATGAAGCAGCTGAAGCGGCGGAGATACACCGGC
TGGGGCAGGCTGAGCCGGAAGCTGATCAACGGCATCCGGGACAAGCAGTCCGGC
AAGACAATCCTGGATTTCCTGAAGTCCGACGGCTTCGCCAACAGAAACTTCATGC
AGCTGATCCACGACGACAGCCTGACCTTTAAAGAGGACATCCAGAAAGCCCAGG
TGTCCGGCCAGGGCGATAGCCTGCACGAGCACATTGCCAATCTGGCCGGCAGCC
CCGCCATTAAGAAGGGCATCCTGCAGACAGTGAAGGTGGTGGACGAGCTCGTGA
AAGTGATGGGCCGGCACAAGCCCGAGAACATCGTGATCGAAATGGCCAGAGAG
AACCAGACCACCCAGAAGGGACAGAAGAACAGCCGCGAGAGAATGAAGCGGAT
CGAAGAGGGCATCAAAGAGCTGGGCAGCCAGATCCTGAAAGAACACCCCGTGG
AAAACACCCAGCTGCAGAACGAGAAGCTGTACCTGTACTACCTGCAGAATGGGC
GGGATATGTACGTGGACCAGGAACTGGACATCAACCGGCTGTCCGACTACGATG
TGGACCATATCGTGCCTCAGAGCTTTCTGAAGGACGACTCCATCGACAACAAGGT
GCTGACCAGAAGCGACAAGAACCGGGGCAAGAGCGACAACGTGCCCTCCGAAG
AGGTCGTGAAGAAGATGAAGAACTACTGGCGGCAGCTGCTGAACGCCAAGCTGA
TTACCCAGAGAAAGTTCGACAATCTGACCAAGGCCGAGAGAGGCGGCCTGAGCG
AACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGA
ATGACAAGCTGATCCGGGAAGTGAAAGTGATCACCCTGAAGTCCAAGCTGGTGT
CCGATTTCCGGAAGGATTTCCAGTTTTACAAAGTGCGCGAGATCAACAACTACCA
CCACGCCCACGACGCCTACCTGAACGCCGTCGTGGGAACCGCCCTGATCAAAAA
GTACCCTAAGCTGGAAAGCGAGTTCGTGTACGGCGACTACAAGGTGTACGACGT
GCGGAAGATGATCGCCAAGAGCGAGCAGGAAATCGGCAAGGCTACCGCCAAGT
ACTTCTTCTACAGCAACATCATGAACTTTTTCAAGACCGAGATTACCCTGGCCAA
CGGCGAGATCCGGAAGCGGCCTCTGATCGAGACAAACGGCGAAACCGGGGAGA
TCGTGTGGGATAAGGGCCGGGATTTTGCCACCGTGCGGAAAGTGCTGAGCATGC
CCCAAGTGAATATCGTGAAAAAGACCGAGGTGCAGACAGGCGGCTTCAGCAAAG
AGTCTATCCTGCCCAAGAGGAACAGCGATAAGCTGATCGCCAGAAAGAAGGACT
GGGACCCTAAGAAGTACGGCGGCTTCGACAGCCCCACCGTGGCCTATTCTGTGCT
GGTGGTGGCCAAAGTGGAAAAGGGCAAGTCCAAGAAACTGAAGAGTGTGAAAG
AGCTGCTGGGGATCACCATCATGGAAAGAAGCAGCTTCGAGAAGAATCCCATCG
ACTTTCTGGAAGCCAAGGGCTACAAAGAAGTGAAAAAGGACCTGATCATCAAGC
TGCCTAAGTACTCCCTGTTCGAGCTGGAAAACGGCCGGAAGAGAATGCTGGCCT
CTGCCGGCGAACTGCAGAAGGGAAACGAACTGGCCCTGCCCTCCAAATATGTGA
ACTTCCTGTACCTGGCCAGCCACTATGAGAAGCTGAAGGGCTCCCCCGAGGATA
ATGAGCAGAAACAGCTGTTTGTGGAACAGCACAAGCACTACCTGGACGAGATCA
TCGAGCAGATCAGCGAGTTCTCCAAGAGAGTGATCCTGGCCGACGCTAATCTGG
ACAAAGTGCTGTCCGCCTACAACAAGCACCGGGATAAGCCCATCAGAGAGCAGG
CCGAGAATATCATCCACCTGTTTACCCTGACCAATCTGGGAGCCCCTGCCGCCTT
CAAGTACTTTGACACCACCATCGACCGGAAGAGGTACACCAGCACCAAAGAGGT
GCTGGACGCCACCCTGATCCACCAGAGCATCACCGGCCTGTACGAGACACGGAT
CGACCTGTCTCAGCTGGGAGGTGACAGCGGCGGGAGCGGCGGGAGCGGGGGGA
GCACTAATCTGAGCGACATCATTGAGAAGGAGACTGGGAAACAGCTGGTCATTC
AGGAGTCCATCCTGATGCTGCCTGAGGAGGTGGAGGAAGTGATCGGCAACAAGC
CAGAGTCTGACATCCTGGTGCACACCGCCTACGACGAGTCCACAGATGAGAATG
TGATGCTGCTGACCTCTGACGCCCCCGAGTATAAGCCTTGGGCCCTGGTCATCCA
GGATTCTAACGGCGAGAATAAGATCAAGATGCTGAGCGGAGGATCCGGAGGATC
TGGAGGCAGCACCAACCTGTCTGACATCATCGAGAAGGAGACAGGCAAGCAGCT
GGTCATCCAGGAGAGCATCCTGATGCTGCCCGAAGAAGTCGAAGAAGTGATCGG
AAACAAGCCTGAGAGCGATATCCTGGTCCATACCGCCTACGACGAGAGTACCGA
CGAAAATGTGATGCTGCTGACATCCGACGCCCCAGAGTATAAGCCCTGGGCTCTG
GTCATCCAGGATTCCAACGGAGAGAACAAAATCAAAATGCTGTCTGGCGGCTCA
AAAAGAACCGCCGACGGCAGCGAATTCGAGCCCAAGAAGAAGAGGAAAGTC
AncBE4max687:
(SEQ ID NO: 47)
ATGAAACGGACAGCCGACGGAAGCGAGTTCGAGTCACCAAAGAAG
AAGCGGAAAGTCTCATCAGAAACAGGACCAGTCGCCGTGGACCCAACACTGAGG
AGAAGGATTGAGCCCCATGAATTTGAAGTCTTTTTCGACCCCAGGGAGCTGAGG
AAGGAGGCATGCCTGCTGTACGAGATCAAGTGGGGCACAAGCCACAAGATCTGG
CGCAACAGCGGCAAGAACACCACAAAGCACGTGGAAGTGAATTTCATCGAGAAG
TTTACCTCCGAGCGGCACTTCTGCCCCTCTATCAGCTGTTCCATCACATGGTTTCT
GTCTTGGAGCCCTTGCTGGGAGTGTTCCAAGGCCATCCGCGAGTTCCTGTCTCAG
CACCCTAACGTGACCCTGGTCATCTACGTGGCCCGGCTGTTTCAACACATGGACC
AGCAGAACAGGCAGGGCCTGCGCGATCTGGTGAATTCTGGCGTGACCATCCAGA
TCATGACAGCCTCAGAGTACGACCATTGCTGGCGGAACTTCGTGAATTATCCACC
TGGCAAGGAGGCACACTGGCCAAGATACCCACCCCTGTGGATGAAGCTGTATGC
ACTGGAGCTGCACGCAGGAATCCTGGGCCTGCCTCCATGTCTGAATATCCTGCGG
AGAAAGCAGCCCCAGCTGACATTTTTCACTATCGCACTGCAGAGCTGTCATTACC
AGAGACTGCCTCCTCATATCCTGTGGGCTACAGGCCTTAAATCTGGAGGATCTAG
CGGAGGATCCTCTGGCAGCGAGACACCAGGAACAAGCGAGTCAGCAACACCAG
AGAGCAGTGGCGGCAGCAGCGGCGGCAGCGACAAGAAGTACAGCATCGGCCTG
GACATCGGCACCAACTCTGTGGGCTGGGCCGTGATCACCGACGAGTACAAGGTG
CCCAGCAAGAAATTCAAGGTGCTGGGCAACACCGACCGGCACAGCATCAAGAAG
AACCTGATCGGAGCCCTGCTGTTCGACAGCGGCGAAACAGCCGAGGCCACCCGG
CTGAAGAGAACCGCCAGAAGAAGATACACCAGACGGAAGAACCGGATCTGCTAT
CTGCAAGAGATCTTCAGCAACGAGATGGCCAAGGTGGACGACAGCTTCTTCCAC
AGACTGGAAGAGTCCTTCCTGGTGGAAGAGGATAAGAAGCACGAGCGGCACCCC
ATCTTCGGCAACATCGTGGACGAGGTGGCCTACCACGAGAAGTACCCCACCATC
TACCACCTGAGAAAGAAACTGGTGGACAGCACCGACAAGGCCGACCTGCGGCTG
ATCTATCTGGCCCTGGCCCACATGATCAAGTTCCGGGGCCACTTCCTGATCGAGG
GCGACCTGAACCCCGACAACAGCGACGTGGACAAGCTGTTCATCCAGCTGGTGC
AGACCTACAACCAGCTGTTCGAGGAAAACCCCATCAACGCCAGCGGCGTGGACG
CCAAGGCCATCCTGTCTGCCAGACTGAGCAAGAGCAGACGGCTGGAAAATCTGA
TCGCCCAGCTGCCCGGCGAGAAGAAGAATGGCCTGTTCGGAAACCTGATTGCCC
TGAGCCTGGGCCTGACCCCCAACTTCAAGAGCAACTTCGACCTGGCCGAGGATG
CCAAACTGCAGCTGAGCAAGGACACCTACGACGACGACCTGGACAACCTGCTGG
CCCAGATCGGCGACCAGTACGCCGACCTGTTTCTGGCCGCCAAGAACCTGTCCGA
CGCCATCCTGCTGAGCGACATCCTGAGAGTGAACACCGAGATCACCAAGGCCCC
CCTGAGCGCCTCTATGATCAAGAGATACGACGAGCACCACCAGGACCTGACCCT
GCTGAAAGCTCTCGTGCGGCAGCAGCTGCCTGAGAAGTACAAAGAGATTTTCTTC
GACCAGAGCAAGAACGGCTACGCCGGCTACATTGACGGCGGAGCCAGCCAGGA
AGAGTTCTACAAGTTCATCAAGCCCATCCTGGAAAAGATGGACGGCACCGAGGA
ACTGCTCGTGAAGCTGAACAGAGAGGACCTGCTGCGGAAGCAGCGGACCTTCGA
CAACGGCAGCATCCCCCACCAGATCCACCTGGGAGAGCTGCACGCCATTCTGCG
GCGGCAGGAAGATTTTTACCCATTCCTGAAGGACAACCGGGAAAAGATCGAGAA
GATCCTGACCTTCCGCATCCCCTACTACGTGGGCCCTCTGGCCAGGGGAAACAGC
AGATTCGCCTGGATGACCAGAAAGAGCGAGGAAACCATCACCCCCTGGAACTTC
GAGGAAGTGGTGGACAAGGGCGCTTCCGCCCAGAGCTTCATCGAGCGGATGACC
AACTTCGATAAGAACCTGCCCAACGAGAAGGTGCTGCCCAAGCACAGCCTGCTG
TACGAGTACTTCACCGTGTATAACGAGCTGACCAAAGTGAAATACGTGACCGAG
GGAATGAGAAAGCCCGCCTTCCTGAGCGGCGAGCAGAAAAAGGCCATCGTGGAC
CTGCTGTTCAAGACCAACCGGAAAGTGACCGTGAAGCAGCTGAAAGAGGACTAC
TTCAAGAAAATCGAGTGCTTCGACTCCGTGGAAATCTCCGGCGTGGAAGATCGGT
TCAACGCCTCCCTGGGCACATACCACGATCTGCTGAAAATTATCAAGGACAAGG
ACTTCCTGGACAATGAGGAAAACGAGGACATTCTGGAAGATATCGTGCTGACCC
TGACACTGTTTGAGGACAGAGAGATGATCGAGGAACGGCTGAAAACCTATGCCC
ACCTGTTCGACGACAAAGTGATGAAGCAGCTGAAGCGGCGGAGATACACCGGCT
GGGGCAGGCTGAGCCGGAAGCTGATCAACGGCATCCGGGACAAGCAGTCCGGC
AAGACAATCCTGGATTTCCTGAAGTCCGACGGCTTCGCCAACAGAAACTTCATGC
AGCTGATCCACGACGACAGCCTGACCTTTAAAGAGGACATCCAGAAAGCCCAGG
TGTCCGGCCAGGGCGATAGCCTGCACGAGCACATTGCCAATCTGGCCGGCAGCC
CCGCCATTAAGAAGGGCATCCTGCAGACAGTGAAGGTGGTGGACGAGCTCGTGA
AAGTGATGGGCCGGCACAAGCCCGAGAACATCGTGATCGAAATGGCCAGAGAG
AACCAGACCACCCAGAAGGGACAGAAGAACAGCCGCGAGAGAATGAAGCGGAT
CGAAGAGGGCATCAAAGAGCTGGGCAGCCAGATCCTGAAAGAACACCCCGTGG
AAAACACCCAGCTGCAGAACGAGAAGCTGTACCTGTACTACCTGCAGAATGGGC
GGGATATGTACGTGGACCAGGAACTGGACATCAACCGGCTGTCCGACTACGATG
TGGACCATATCGTGCCTCAGAGCTTTCTGAAGGACGACTCCATCGACAACAAGGT
GCTGACCAGAAGCGACAAGAACCGGGGCAAGAGCGACAACGTGCCCTCCGAAG
AGGTCGTGAAGAAGATGAAGAACTACTGGCGGCAGCTGCTGAACGCCAAGCTGA
TTACCCAGAGAAAGTTCGACAATCTGACCAAGGCCGAGAGAGGCGGCCTGAGCG
AACTGGATAAGGCCGGCTTCATCAAGAGACAGCTGGTGGAAACCCGGCAGATCA
CAAAGCACGTGGCACAGATCCTGGACTCCCGGATGAACACTAAGTACGACGAGA
ATGACAAGCTGATCCGGGAAGTGAAAGTGATCACCCTGAAGTCCAAGCTGGTGT
CCGATTTCCGGAAGGATTTCCAGTTTTACAAAGTGCGCGAGATCAACAACTACCA
CCACGCCCACGACGCCTACCTGAACGCCGTCGTGGGAACCGCCCTGATCAAAAA
GTACCCTAAGCTGGAAAGCGAGTTCGTGTACGGCGACTACAAGGTGTACGACGT
GCGGAAGATGATCGCCAAGAGCGAGCAGGAAATCGGCAAGGCTACCGCCAAGT
ACTTCTTCTACAGCAACATCATGAACTTTTTCAAGACCGAGATTACCCTGGCCAA
CGGCGAGATCCGGAAGCGGCCTCTGATCGAGACAAACGGCGAAACCGGGGAGA
TCGTGTGGGATAAGGGCCGGGATTTTGCCACCGTGCGGAAAGTGCTGAGCATGC
CCCAAGTGAATATCGTGAAAAAGACCGAGGTGCAGACAGGCGGCTTCAGCAAAG
AGTCTATCCTGCCCAAGAGGAACAGCGATAAGCTGATCGCCAGAAAGAAGGACT
GGGACCCTAAGAAGTACGGCGGCTTCGACAGCCCCACCGTGGCCTATTCTGTGCT
GGTGGTGGCCAAAGTGGAAAAGGGCAAGTCCAAGAAACTGAAGAGTGTGAAAG
AGCTGCTGGGGATCACCATCATGGAAAGAAGCAGCTTCGAGAAGAATCCCATCG
ACTTTCTGGAAGCCAAGGGCTACAAAGAAGTGAAAAAGGACCTGATCATCAAGC
TGCCTAAGTACTCCCTGTTCGAGCTGGAAAACGGCCGGAAGAGAATGCTGGCCT
CTGCCGGCGAACTGCAGAAGGGAAACGAACTGGCCCTGCCCTCCAAATATGTGA
ACTTCCTGTACCTGGCCAGCCACTATGAGAAGCTGAAGGGCTCCCCCGAGGATA
ATGAGCAGAAACAGCTGTTTGTGGAACAGCACAAGCACTACCTGGACGAGATCA
TCGAGCAGATCAGCGAGTTCTCCAAGAGAGTGATCCTGGCCGACGCTAATCTGG
ACAAAGTGCTGTCCGCCTACAACAAGCACCGGGATAAGCCCATCAGAGAGCAGG
CCGAGAATATCATCCACCTGTTTACCCTGACCAATCTGGGAGCCCCTGCCGCCTT
CAAGTACTTTGACACCACCATCGACCGGAAGAGGTACACCAGCACCAAAGAGGT
GCTGGACGCCACCCTGATCCACCAGAGCATCACCGGCCTGTACGAGACACGGAT
CGACCTGTCTCAGCTGGGAGGTGACAGCGGCGGGAGCGGCGGGAGCGGGGGGA
GCACTAATCTGAGCGACATCATTGAGAAGGAGACTGGGAAACAGCTGGTCATTC
AGGAGTCCATCCTGATGCTGCCTGAGGAGGTGGAGGAAGTGATCGGCAACAAGC
CAGAGTCTGACATCCTGGTGCACACCGCCTACGACGAGTCCACAGATGAGAATG
TGATGCTGCTGACCTCTGACGCCCCCGAGTATAAGCCTTGGGCCCTGGTCATCCA
GGATTCTAACGGCGAGAATAAGATCAAGATGCTGAGCGGAGGATCCGGAGGATC
TGGAGGCAGCACCAACCTGTCTGACATCATCGAGAAGGAGACAGGCAAGCAGCT
GGTCATCCAGGAGAGCATCCTGATGCTGCCCGAAGAAGTCGAAGAAGTGATCGG
AAACAAGCCTGAGAGCGATATCCTGGTCCATACCGCCTACGACGAGAGTACCGA
CGAAAATGTGATGCTGCTGACATCCGACGCCCCAGAGTATAAGCCCTGGGCTCTG
GTCATCCAGGATTCCAACGGAGAGAACAAAATCAAAATGCTGTCTGGCGGCTCA
AAAAGAACCGCCGACGGCAGCGAATTCGAGCCCAAGAAGAAGAGGAAAGTC
ABEmax
(SEQ ID NO: 48)
ATGAAACGGACAGCCGACGGAAGCGAGTTCGAGTCACCAAAGAAG
AAGCGGAAAGTCTCTGAAGTCGAGTTTAGCCACGAGTATTGGATGAGGCACGCA
CTGACCCTGGCAAAGCGAGCATGGGATGAAAGAGAAGTCCCCGTGGGCGCCGTG
CTGGTGCACAACAATAGAGTGATCGGAGAGGGATGGAACAGGCCAATCGGCCGC
CACGACCCTACCGCACACGCAGAGATCATGGCACTGAGGCAGGGAGGCCTGGTC
ATGCAGAATTACCGCCTGATCGATGCCACCCTGTATGTGACACTGGAGCCATGCG
TGATGTGCGCAGGAGCAATGATCCACAGCAGGATCGGAAGAGTGGTGTTCGGAG
CACGGGACGCCAAGACCGGCGCAGCAGGCTCCCTGATGGATGTGCTGCACCACC
CCGGCATGAACCACCGGGTGGAGATCACAGAGGGAATCCTGGCAGACGAGTGCG
CCGCCCTGCTGAGCGATTTCTTTAGAATGCGGAGACAGGAGATCAAGGCCCAGA
AGAAGGCACAGAGCTCCACCGACTCTGGAGGATCTAGCGGAGGATCCTCTGGAA
GCGAGACACCAGGCACAAGCGAGTCCGCCACACCAGAGAGCTCCGGCGGCTCCT
CCGGAGGATCCTCTGAGGTGGAGTTTTCCCACGAGTACTGGATGAGACATGCCCT
GACCCTGGCCAAGAGGGCACGCGATGAGAGGGAGGTGCCTGTGGGAGCCGTGCT
GGTGCTGAACAATAGAGTGATCGGCGAGGGCTGGAACAGAGCCATCGGCCTGCA
CGACCCAACAGCCCATGCCGAAATTATGGCCCTGAGACAGGGCGGCCTGGTCAT
GCAGAACTACAGACTGATTGACGCCACCCTGTACGTGACATTCGAGCCTTGCGTG
ATGTGCGCCGGCGCCATGATCCACTCTAGGATCGGCCGCGTGGTGTTTGGCGTGA
GGAACGCAAAAACCGGCGCCGCAGGCTCCCTGATGGACGTGCTGCACTACCCCG
GCATGAATCACCGCGTCGAAATTACCGAGGGAATCCTGGCAGATGAATGTGCCG
CCCTGCTGTGCTATTTCTTTCGGATGCCTAGACAGGTGTTCAATGCTCAGAAGAA
GGCCCAGAGCTCCACCGACTCCGGAGGATCTAGCGGAGGCTCCTCTGGCTCTGA
GACACCTGGCACAAGCGAGAGCGCAACACCTGAAAGCAGCGGGGGCAGCAGCG
GGGGGTCAGACAAGAAGTACAGCATCGGCCTGGCCATCGGCACCAACTCTGTGG
GCTGGGCCGTGATCACCGACGAGTACAAGGTGCCCAGCAAGAAATTCAAGGTGC
TGGGCAACACCGACCGGCACAGCATCAAGAAGAACCTGATCGGAGCCCTGCTGT
TCGACAGCGGCGAAACAGCCGAGGCCACCCGGCTGAAGAGAACCGCCAGAAGA
AGATACACCAGACGGAAGAACCGGATCTGCTATCTGCAAGAGATCTTCAGCAAC
GAGATGGCCAAGGTGGACGACAGCTTCTTCCACAGACTGGAAGAGTCCTTCCTG
GTGGAAGAGGATAAGAAGCACGAGCGGCACCCCATCTTCGGCAACATCGTGGAC
GAGGTGGCCTACCACGAGAAGTACCCCACCATCTACCACCTGAGAAAGAAACTG
GTGGACAGCACCGACAAGGCCGACCTGCGGCTGATCTATCTGGCCCTGGCCCAC
ATGATCAAGTTCCGGGGCCACTTCCTGATCGAGGGCGACCTGAACCCCGACAAC
AGCGACGTGGACAAGCTGTTCATCCAGCTGGTGCAGACCTACAACCAGCTGTTCG
AGGAAAACCCCATCAACGCCAGCGGCGTGGACGCCAAGGCCATCCTGTCTGCCA
GACTGAGCAAGAGCAGACGGCTGGAAAATCTGATCGCCCAGCTGCCCGGCGAGA
AGAAGAATGGCCTGTTCGGAAACCTGATTGCCCTGAGCCTGGGCCTGACCCCCA
ACTTCAAGAGCAACTTCGACCTGGCCGAGGATGCCAAACTGCAGCTGAGCAAGG
ACACCTACGACGACGACCTGGACAACCTGCTGGCCCAGATCGGCGACCAGTACG
CCGACCTGTTTCTGGCCGCCAAGAACCTGTCCGACGCCATCCTGCTGAGCGACAT
CCTGAGAGTGAACACCGAGATCACCAAGGCCCCCCTGAGCGCCTCTATGATCAA
GAGATACGACGAGCACCACCAGGACCTGACCCTGCTGAAAGCTCTCGTGCGGCA
GCAGCTGCCTGAGAAGTACAAAGAGATTTTCTTCGACCAGAGCAAGAACGGCTA
CGCCGGCTACATTGACGGCGGAGCCAGCCAGGAAGAGTTCTACAAGTTCATCAA
GCCCATCCTGGAAAAGATGGACGGCACCGAGGAACTGCTCGTGAAGCTGAACAG
AGAGGACCTGCTGCGGAAGCAGCGGACCTTCGACAACGGCAGCATCCCCCACCA
GATCCACCTGGGAGAGCTGCACGCCATTCTGCGGCGGCAGGAAGATTTTTACCCA
TTCCTGAAGGACAACCGGGAAAAGATCGAGAAGATCCTGACCTTCCGCATCCCC
TACTACGTGGGCCCTCTGGCCAGGGGAAACAGCAGATTCGCCTGGATGACCAGA
AAGAGCGAGGAAACCATCACCCCCTGGAACTTCGAGGAAGTGGTGGACAAGGGC
GCTTCCGCCCAGAGCTTCATCGAGCGGATGACCAACTTCGATAAGAACCTGCCCA
ACGAGAAGGTGCTGCCCAAGCACAGCCTGCTGTACGAGTACTTCACCGTGTATA
ACGAGCTGACCAAAGTGAAATACGTGACCGAGGGAATGAGAAAGCCCGCCTTCC
TGAGCGGCGAGCAGAAAAAGGCCATCGTGGACCTGCTGTTCAAGACCAACCGGA
AAGTGACCGTGAAGCAGCTGAAAGAGGACTACTTCAAGAAAATCGAGTGCTTCG
ACTCCGTGGAAATCTCCGGCGTGGAAGATCGGTTCAACGCCTCCCTGGGCACATA
CCACGATCTGCTGAAAATTATCAAGGACAAGGACTTCCTGGACAATGAGGAAAA
CGAGGACATTCTGGAAGATATCGTGCTGACCCTGACACTGTTTGAGGACAGAGA
GATGATCGAGGAACGGCTGAAAACCTATGCCCACCTGTTCGACGACAAAGTGAT
GAAGCAGCTGAAGCGGCGGAGATACACCGGCTGGGGCAGGCTGAGCCGGAAGC
TGATCAACGGCATCCGGGACAAGCAGTCCGGCAAGACAATCCTGGATTTCCTGA
AGTCCGACGGCTTCGCCAACAGAAACTTCATGCAGCTGATCCACGACGACAGCC
TGACCTTTAAAGAGGACATCCAGAAAGCCCAGGTGTCCGGCCAGGGCGATAGCC
TGCACGAGCACATTGCCAATCTGGCCGGCAGCCCCGCCATTAAGAAGGGCATCC
TGCAGACAGTGAAGGTGGTGGACGAGCTCGTGAAAGTGATGGGCCGGCACAAGC
CCGAGAACATCGTGATCGAAATGGCCAGAGAGAACCAGACCACCCAGAAGGGA
CAGAAGAACAGCCGCGAGAGAATGAAGCGGATCGAAGAGGGCATCAAAGAGCT
GGGCAGCCAGATCCTGAAAGAACACCCCGTGGAAAACACCCAGCTGCAGAACG
AGAAGCTGTACCTGTACTACCTGCAGAATGGGCGGGATATGTACGTGGACCAGG
AACTGGACATCAACCGGCTGTCCGACTACGATGTGGACCATATCGTGCCTCAGAG
CTTTCTGAAGGACGACTCCATCGACAACAAGGTGCTGACCAGAAGCGACAAGAA
CCGGGGCAAGAGCGACAACGTGCCCTCCGAAGAGGTCGTGAAGAAGATGAAGA
ACTACTGGCGGCAGCTGCTGAACGCCAAGCTGATTACCCAGAGAAAGTTCGACA
ATCTGACCAAGGCCGAGAGAGGCGGCCTGAGCGAACTGGATAAGGCCGGCTTCA
TCAAGAGACAGCTGGTGGAAACCCGGCAGATCACAAAGCACGTGGCACAGATCC
TGGACTCCCGGATGAACACTAAGTACGACGAGAATGACAAGCTGATCCGGGAAG
TGAAAGTGATCACCCTGAAGTCCAAGCTGGTGTCCGATTTCCGGAAGGATTTCCA
GTTTTACAAAGTGCGCGAGATCAACAACTACCACCACGCCCACGACGCCTACCT
GAACGCCGTCGTGGGAACCGCCCTGATCAAAAAGTACCCTAAGCTGGAAAGCGA
GTTCGTGTACGGCGACTACAAGGTGTACGACGTGCGGAAGATGATCGCCAAGAG
CGAGCAGGAAATCGGCAAGGCTACCGCCAAGTACTTCTTCTACAGCAACATCAT
GAACTTTTTCAAGACCGAGATTACCCTGGCCAACGGCGAGATCCGGAAGCGGCC
TCTGATCGAGACAAACGGCGAAACCGGGGAGATCGTGTGGGATAAGGGCCGGG
ATTTTGCCACCGTGCGGAAAGTGCTGAGCATGCCCCAAGTGAATATCGTGAAAA
AGACCGAGGTGCAGACAGGCGGCTTCAGCAAAGAGTCTATCCTGCCCAAGAGGA
ACAGCGATAAGCTGATCGCCAGAAAGAAGGACTGGGACCCTAAGAAGTACGGC
GGCTTCGACAGCCCCACCGTGGCCTATTCTGTGCTGGTGGTGGCCAAAGTGGAAA
AGGGCAAGTCCAAGAAACTGAAGAGTGTGAAAGAGCTGCTGGGGATCACCATCA
TGGAAAGAAGCAGCTTCGAGAAGAATCCCATCGACTTTCTGGAAGCCAAGGGCT
ACAAAGAAGTGAAAAAGGACCTGATCATCAAGCTGCCTAAGTACTCCCTGTTCG
AGCTGGAAAACGGCCGGAAGAGAATGCTGGCCTCTGCCGGCGAACTGCAGAAGG
GAAACGAACTGGCCCTGCCCTCCAAATATGTGAACTTCCTGTACCTGGCCAGCCA
CTATGAGAAGCTGAAGGGCTCCCCCGAGGATAATGAGCAGAAACAGCTGTTTGT
GGAACAGCACAAGCACTACCTGGACGAGATCATCGAGCAGATCAGCGAGTTCTC
CAAGAGAGTGATCCTGGCCGACGCTAATCTGGACAAAGTGCTGTCCGCCTACAA
CAAGCACCGGGATAAGCCCATCAGAGAGCAGGCCGAGAATATCATCCACCTGTT
TACCCTGACCAATCTGGGAGCCCCTGCCGCCTTCAAGTACTTTGACACCACCATC
GACCGGAAGAGGTACACCAGCACCAAAGAGGTGCTGGACGCCACCCTGATCCAC
CAGAGCATCACCGGCCTGTACGAGACACGGATCGACCTGTCTCAGCTGGGAGGT
GACTCTGGCGGCTCAAAAAGAACCGCCGACGGCAGCGAATTCGAGCCCAAGAAG
AAGAGGAAAGTC
SCN9a RNA:
(SEQ ID NO: 49)
GUUAGUCCUUAAAAUGUAGGG
MPDU1 RNA:
(SEQ ID NO: 50)
GUUCCCGGUCAUGCACUACAG
HBG site 1 RNA:
(SEQ ID NO: 51)
CUUGACCAAUAGCCUUGACA
HBG site 2 RNA:
(SEQ ID NO: 52)
AUAUUUGCAUUGAGAUAGUG

### REFERENCES

1. Landrum. M. J. et al. Clin Var: public archive of interpretations of clinically relevant variants. Nucleic acids research 44. D862-868 (2016).
2. Komor, A. C., Kim, Y. B., Packer, M. S., Zuris, J. A. & Liu, D. R. Programmable editing of a target base in genomic DNA without double-stranded DNA cleavage. Nature 533, 420-+ (2016).
3. Gaudelli, N. M. et al. Programmable base editing of A·T to G·C in genomic DNA without DNA cleavage. Nature 551, 464-+ (2017).
4. Shimatani, Z. et al. Targeted base editing in rice and tomato using a CRISPR-Cas9 cytidine deaminase fusion. Nature biotechnology 35, 441-443 (2017).
5. Nishida, K. et al. Targeted nucleotide editing using hybrid prokaryotic and vertebrate adaptive immune systems. Science 353 (2016).
6. Komor, A. C. et al. Improved base excision repair inhibition and bacteriophage Mu Gam protein yields C: G-to-T: A base editors with higher efficiency and product purity. Sci Adv 3, eaao4774 (2017).
7. Komor, A. C., Badran, A. H. & Cell, L.-D. R. CRISPR-based technologies for the manipulation of eukaryotic genomes. Cell (2017).
8. Satomura, A. et al. Precise genome-wide base editing by the CRISPR Nickase system in yeast. Sci Rep 7, 2095 (2017).
9. Zhang, Y. et al. Programmable base editing of zebrafish genome using a modified CRISPR-Cas9 system. Nat Commun 8, 118 (2017).
10. Lu, Y. M. & Zhu, J. K. Precise Editing of a Target Base in the Rice Genome Using a Modified CRISPR/Cas9 System. Molecular Plant 10, 523-525 (2017).
11. Zong, Y. et al. Precise base editing in rice, wheat and maize with a Cas9-cytidine deaminase fusion. Nature biotechnology 35, 438-440 (2017).
12. Billon, P. et al. CRISPR-Mediated Base Editing Enables Efficient Disruption of Eukaryotic Genes through Induction of STOP Codons. Molecular Cell 67, 1068-+ (2017).
13. Kuscu, C. et al. CRISPR-STOP: gene silencing through base-editing-induced nonsense mutations. Nature methods 14, 710-712 (2017).
14. Kim, K. et al. Highly efficient RNA-guided base editing in mouse embryos. Nature biotechnology 35, 435-437 (2017).
15. Chadwick, A. C., Wang, X. & Musunuru, K. In Vivo Base Editing of PCSK9 (Proprotein Convertase Subtilisin/Kexin Type 9) as a Therapeutic Alternative to Genome Editing. Arterioscler Thromb Vasc Biol 37, 1741-1747 (2017).
16, Li, G. et al. Highly efficient and precise base editing in discarded human tripronuclear embryos. Protein Cell 8, 776-779 (2017).
17. Liang, P. et al. Correction of beta-thalassemia mutant by base editor in human embryos. Protein Cell 8, 811-822 (2017).
18. Ryu, S. M. et al. Adenine base editing in mouse embryos and an adult mouse model of Duchenne muscular dystrophy. Nature biotechnology (2017).
19. Kim, Y. B. et al. Increasing the genome-targeting scope and precision of base editing with engineered Cas9-cytidine deaminase fusions. Nature biotechnology 35, 371-376 (2017).
20. Hu, J. H. et al. Evolved Cas9 variants with broad PAM compatibility and high DNA specificity. Nature 556, 57-63 (2018).
21. Li, X. et al. Base editing with a Cpf1-cytidine deaminase fusion. Nature biotechnology 36, 324-327 (2018).
22. Rees, H. A. et al. Improving the DNA specificity and applicability of base editing through protein engineering and protein delivery. Nat Commun 8, 15790 (2017).
23. Kleinstiver, B. P., Pattanayak, V., Prew, M. S. & Nature, T.-S. Q. High-fidelity CRISPR-Cas9 nucleases with no detectable genome-wide off-target effects. Nature (2016).
24. Chen, J. S. et al. Enhanced proofreading governs CRISPR-Cas9 targeting accuracy. Nature 550, 407-410 (2017).
25. Slaymaker, I. M. et al. Rationally engineered Cas9 nucleases with improved specificity. Science 351, 84-88 (2016).
26. Wang, T., Badran, A. H., Huang, T. P., Liu, D. R. Continuous directed evolution of proteins with improved soluble expression In Review. (2018).
27. Kim, J. H. et al. High cleavage efficiency of a 2A peptide derived from porcine teschovirus-1 in human cell lines, zebrafish and mice. PloS one 6, e18556 (2011).
28. Suzuki, K. et al. In vivo genome editing via CRISPR/Cas9 mediated homology-independent targeted integration. Nature 540, 144-149 (2016).
29. Hanson, G. & Coller, J. Codon optimality, bias and usage in translation and mRNA decay. Nature reviews. Molecular cell biology 19, 20-30 (2018).
30. Kim, S., Bae, T., Hwang, J. & Kim, J. S. Rescue of high-specificity Cas9 variants using sgRNAs with matched 5′ nucleotides. Genome biology 18, 218 (2017).
31. Harms, M. J. & Thornton, J. W. Evolutionary biochemistry: revealing the historical and physical causes of protein properties. Nature reviews. Genetics 14, 559-571 (2013).
32. Wheeler, L. C., Lim, S. A., Marqusee, S. & Harms, M. J. The thermostability and specificity of ancient proteins. Curr Opin Struct Biol 38, 37-43 (2016).
33. Nguyen, V. et al. Evolutionary drivers of thermoadaptation in enzyme catalysis. Science 355, 289-294 (2017).
34. Wilson, C. et al. Kinase dynamics. Using ancient protein kinases to unravel a modern cancer drug's mechanism. Science 347, 882-886 (2015).
35. Risso, V. A., Gavira, J. A., Mejia-Carmona, D. F., Gaucher, E. A. & Sanchez-Ruiz, J. M. Hyperstability and substrate promiscuity in laboratory resurrections of Precambrian beta-lactamases. J Am Chem Soc 135, 2899-2902 (2013).
36. Williams, P. D., Pollock, D. D., Blackburne, B. P. & Goldstein, R. A. Assessing the accuracy of ancestral protein reconstruction methods. PLOS Comput Biol 2, e69 (2006).
37. Trudeau, D. L., Kaltenbach, M. & Tawfik, D. S. On the Potential Origins of the High Stability of Reconstructed Ancestral Proteins. Mol Biol Evol 33, 2633-2641 (2016).
38. Eick, G. N., Bridgham, J. T., Anderson, D. P., Harms, M. J. & Thornton, J. W. Robustness of reconstructed ancestral protein functions to statistical uncertainty. Molecular biology and evolution (2016).
39. Gumulya, Y. & Gillam, E. M. Exploring the past and the future of protein evolution with ancestral sequence reconstruction: the ‘retro’ approach to protein engineering. Biochem J 474, 1-19 (2017).
40. Megan F. Cole, V. E. C., Kelsey L. Gratton, and Eric A. Gaucher Reconstructing Evolutionary Adaptive Paths for Protein Engineering. Enyme Engineering Methods and Protocols 978 (2013).
41. Zakas, P. M. et al. Enhancing the pharmaceutical properties of protein drugs by ancestral sequence reconstruction. Nature biotechnology 35, 35-37 (2017).
42. Krokan, H. E., Drablos, F. & Slupphaug, G. Uracil in DNA—occurrence, consequences and repair. Oncogene 21, 8935-8948 (2002).
43. Schenk, B. et al. MPDU1 mutations underlie a novel human congenital disorder of glycosylation, designated type If. The Journal of clinical investigation 108, 1687-1695 (2001).
44. Bennett, D. L. & Woods, C·G. Painful and painless channelopathies. The Lancet. Neurology 13, 587-599 (2014).
45. Waxman, S. G. & Zamponi, G. W. Regulating excitability of peripheral afferents: emerging ion channel targets. Nature neuroscience 17, 153-163 (2014).
46. Traxler, E. A. et al. A genome-editing strategy to treat beta-hemoglobinopathies that recapitulates a mutation associated with a benign genetic condition. Nature medicine 22, 987-990 (2016).
47. Liu, N. et al. Direct Promoter Repression by BCL11A Controls the Fetal to Adult Hemoglobin Switch. Cell 173, 430-442 e417 (2018).
48. Amato, A. et al. Interpreting elevated fetal hemoglobin in pathology and health at the basic laboratory level: new and known gamma-gene mutations associated with hereditary persistence of fetal hemoglobin. International journal of laboratory hematology 36, 13-19 (2014).
49. Badran, A. H. et al. Continuous evolution of Bacillus thuringiensis toxins overcomes insect resistance. Nature 533, 58-63 (2016).
50. Gibson, D. G. et al. Enzymatic assembly of DNA molecules up to several hundred kilobases. Nature methods 6, 343-345 (2009).
51. Li, H. & Durbin, R. Fast and accurate short read alignment with Burrows-Wheeler transform. Bioinformatics 25, 1754-1760 (2009).
52. UniProt Consortium, T. UniProt: the universal protein knowledgebase. Nucleic acids research 46, 2699 (2018).
53. Altschul, S. F., Gish, W., Miller, W., Myers, E. W. & Lipman, D.J. Basic local alignment search tool. Journal of molecular biology 215, 403-410 (1990).
54. Katoh, K. & Standley, D. M. MAFFT multiple sequence alignment software version 7: improvements in performance and usability. Molecular biology and evolution 30, 772-780 (2013).
55. Kalyaanamoorthy, S., Minh, B. Q., Wong, T. K. F., von Haeseler, A. & Jermiin, L. S. ModelFinder: fast model selection for accurate phylogenetic estimates. Nature methods 14, 587-589 (2017).
56. Nguyen, L. T., Schmidt, H. A., von Haeseler, A. & Minh, B. Q. IQ-TREE: a fast and effective stochastic algorithm for estimating maximum-likelihood phylogenies. Mol Biol Evol 32, 268-274 (2015).
57. Hoang, D. T., Chernomor, O., von Haeseler, A., Minh, B. Q. & Vinh, L. S. UFBoot2: Improving the Ultrafast Bootstrap Approximation. Mol Biol Evol 35, 518-522 (2018).
58. Yang, Z. PAML 4: phylogenetic analysis by maximum likelihood.

Molecular biology and evolution 24, 1586-1591 (2007).

### OTHER EMBODIMENTS

The foregoing has been a description of certain non-limiting embodiments of the invention. Those of ordinary skill in the art will appreciate that various changes and modifications to this description may be made without departing from the spirit or scope of the present invention, as defined in the following claims.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1.-129. (canceled)

130. A cytidine deaminase comprising an amino acid sequence that is at least 80% identical to the amino acid sequence of any one of SEQ ID NOs: 6-9.

131. The cytidine deaminase of claim 130, wherein the cytidine deaminase comprises an amino acid sequence set forth in any one of SEQ ID NOs: 6-9.

132.-147. (canceled)

148. A fusion protein comprising (i) a nucleic acid programmable DNA binding protein (napDNAbp); and (ii) the cytidine deaminase of claim 130.

149. The fusion protein of claim 148, wherein the fusion protein further comprises (iii) one or more UGI domains.

150. The fusion protein of claim 148, wherein the fusion protein further comprises (iv) one or more nuclear localization sequences.

151. The fusion protein of claim 148, wherein the nucleic acid programmable DNA binding protein is a Cas9 domain.

152. The fusion protein of claim 151, wherein the Cas9 domain is a Cas9 nickase domain.

153. The fusion protein of claim 152, wherein the Cas9 nickase domain cuts a nucleic acid target strand of a nucleotide duplex, wherein the nucleotide target strand is the strand that binds a gRNA.

154. The fusion protein of claim 151, wherein the Cas9 domain comprises an amino acid sequence that is at least 80% identical to the amino acid sequence of SEQ ID NO: 3 .

155. The fusion protein of claim 151, wherein the Cas9 domain comprises the amino acid sequence of SEQ ID NO: 3 .

156. The fusion protein of claim 149, wherein the fusion protein comprises two UGI domains.

157. The fusion protein of claim 149, wherein the one or more UGI domains comprise an amino acid sequence that is at least 80%, 85%, 90%, 95%, 98%, or 99% identical to the amino acid sequence of TNLSDIIEKETGKQLVIQESILMLPEEVEEVIGNKPESDILVHTAYDESTDENVMLLTSDAPE YKPWALVIQDSNGENKIKML (SEQ ID NO: 108).

158. The fusion protein of claim 149, wherein the one or more UGI domains comprise the amino acid sequence of SEQ ID NO: 108.

159. The fusion protein of claim 148, wherein the fusion protein comprises the structure:
(i) NH₂-[cytidine deaminase of claim 130]-[Cas9 domain]-COOH;
(ii) NH₂-[cytidine deaminase of claim 130]-[Cas9 domain]-[UGI domain]-COOH;
(iii) NH₂-[cytidine deaminase of claim 130]-[Cas9 domain]-[first UGI domain]-[second UGI domain]-COOH;
(iv) NH₂-[cytidine deaminase of claim 130]-[Cas9 domain]-[nuclear localization sequence]-COOH;
(v) NH₂-[first nuclear localization sequence]-[cytidine deaminase of claim 130]-[Cas9 domain]-[second nuclear localization sequence]-COOH; or
(vi) NH₂-[first nuclear localization sequence]-[cytidine deaminase of claim 130]-[Cas9 domain]-[first UGI domain]-[second UGI domain]-[second nuclear localization sequence]-COOH; wherein each instance of “-” comprises an optional linker.

160.-166. (canceled)

167. The fusion protein of claim 150, wherein the one or more nuclear localization sequences are bipartite nuclear localization sequences.

168. The fusion protein of claim 150, wherein the one or more nuclear localization sequences comprises the amino acid sequence KRTADGSEFESPKKKRKV (SEQ ID NO: 1) or KRTADGSEFEPKKKRKV (SEQ ID NO: 2).

169. The fusion protein of claim 150, wherein the fusion protein comprises a first nuclear localization sequence and a second nuclear localization sequence, and wherein the first nuclear localization sequence comprises the amino acid sequence KRTADGSEFESPKKKRKV (SEQ ID NO: 1), and the second nuclear localization sequence comprises the amino acid sequence KRTADGSEFEPKKKRKV (SEQ ID NO: 2).

170. The fusion protein of claim 148, wherein the fusion protein comprises an amino acid sequence that is at least 80% identical to the amino acid sequence of any one of SEQ ID NOs: 12, 13, or 14.

171. The fusion protein of claim 148, wherein the fusion protein comprises the amino acid sequence of any one of SEQ ID NOs: 12, 13, or 14.

172. A fusion protein comprising (i) a nucleic acid programmable DNA binding protein (napDNAbp); and (ii) the cytidine deaminase of claim 131.
